(19) United States
(12) Patent Application Publication
(10) Pub. No.: US 2026/0008783 A1
(43) Pub. Date: Jan. 8, 2026

(54) MODULATORS OF FSHR AND USES THEREOF

(71) Applicant: Celmatix Inc., New York, NY (US)

(72) Inventors: Stephen S. PALMER, Groton, MA (US); Robert L. DOW, Groton, CT (US)

(21) Appl. No.: 19/060,992

(22) Filed: Feb. 24, 2025

Related U.S. Application Data
(63) Continuation of application No. PCT/US2023/072957, filed on Aug. 25, 2023.
(60) Provisional application No. 63/401,441, filed on Aug. 26, 2022.
(60) Provisional application No. 63/522,983, filed on Jun. 23, 2023.

(51) Int. Cl. C07D 491/052; C07D 231/54; C07D 231/56; C07D 401/04; C07D 401/14; C07D 409/14; C07D 413/06; C07D 413/14; C07D 471/04; C07D 487/04; C07D 487/08; C07D 487/10; C07D 491/048; C07D 491/107; C07D 495/04; C07D 498/08

(52) U.S. Cl. CPC C07D 491/052; C07D 231/54; C07D 231/56; C07D 401/04; C07D 401/14; C07D 409/14; C07D 413/06; C07D 413/14; C07D 471/04; C07D 487/04; C07D 487/08; C07D 487/10; C07D 491/048; C07D 491/107; C07D 495/04; C07D 498/08

(57) ABSTRACT

Disclosed herein are novel FSH modulator compounds, formulations thereof, and methods of treating diseases by administration of one or more novel FSH modulator compounds.

### CROSS-REFERENCE

This application is a continuation of International Application No. PCT/US2023/072957, filed Aug. 25, 2023, which claims the benefit of U.S. Provisional Application No. 63/401,441 filed Aug. 26, 2022 and U.S. Provisional Application No. 63/522,983 filed Jun. 23, 2023 all of which are incorporated herein by reference in their entireties.

### BACKGROUND

Glycoprotein hormones (e.g. gonadotropins and/or TSH) serve important functions in a variety of bodily functions including metabolism, temperature regulation and the reproductive process. Gonadotropins act on specific gonadal cell types to initiate ovarian and testicular differentiation and steroidogenesis. The gonadotropin FSH (follicle stimulating hormone) is released from the anterior pituitary under the influence of gonadotropin-releasing hormone and estrogens, and from the placenta during pregnancy. FSH is a heterodimeric glycoprotein hormone that shares structural similarities with luteinizing hormone (LH) and thyroid stimulating hormone (TSH), both of which are also produced in the pituitary gland, and chorionic gonadotropin (CG), which is produced in the placenta. In the female, FSH plays a pivotal role in the stimulation of follicle development and maturation and in addition, it is the major hormone regulating synthesis of estrogens, whereas LH induces ovulation. In the male, FSH is responsible for the integrity of the seminiferous tubules and acts on Sertoli cells to support gametogenesis.

The hormones are relatively large (28-38 kDa) and are composed of a common α-subunit non-covalently bound to a distinct β-subunit that confers receptor binding specificity. The cellular receptor for these hormones is expressed on testicular Sertoli cells and ovarian granulosa cells. The FSH receptor is known to be members of the G protein-coupled class of membrane-bound receptors, which when activated stimulate an increase in the activity of adenylyl cyclase. This results in an increase in the level of the intracellular second messenger adenosine 3′, 5′-monophosphate (cAMP), which in turn causes increased steroid synthesis and secretion. Recent research on FSH receptor has shown that intracellular signaling extends beyond the classic Gs-mediated adenylyl cyclase (cAMP) and includes Gi, Gq proteins and changes in intracellular calcium, and serine/threonine kinases (e.g. MAPK, Akt). Hydropathicity plots of the amino acid sequences of these receptors reveal three general domains: a hydrophilic amino-terminal region, considered to be the amino-terminal extracellular domain, that includes a hinge domain that serves as a tethered inverse agonist; seven hydrophobic segments of membrane-spanning length, considered to be the transmembrane domain; and a carboxy-terminal region that contains potential phosphorylation sites (serine, threonine, and tyrosine residues), considered to be the carboxy-terminal intracellular or cytoplasmic domain. The glycoprotein hormone receptor family is distinguished from other G protein-coupled receptors, such as the β-2-adrenergic, rhodopsin, and substance K receptors, by the large size of the hydrophilic amino-terminal domain, which is involved in hormone binding.

Annually in the U.S. there are millions of couples experiencing infertility that are potential candidates for treatment. FSH, either extracted from urine or produced by recombinant DNA technology, is a parenterally-administered protein product used by specialists for ovulation induction and for controlled ovarian hyperstimulation. Whereas ovulation induction is directed at achieving a single follicle to ovulate, controlled ovarian hyperstimulation is directed at harvesting multiple oocytes for use in various in-vitro assisted reproductive technologies, e.g. in-vitro fertilization (IVF). FSH is also used clinically to treat male hypogonadism and male non-obstructive infertility, e.g. some types of failure of spermatogenesis.

FSHR is a highly specific target in the ovarian follicle growth process and is exclusively expressed in the ovary. However, the use of FSH is limited by its high cost, lack of oral dosing, and need of extensive monitoring by specialist physicians. Hence, identification of a non-peptidic small molecule substitute for FSH that could be developed for oral administration is desirable. There is still a need for low molecular weight hormone mimetics that selectively modulate FSHR.

### BRIEF SUMMARY

In one aspect, described herein are FSH modulator compound of Formula (I):

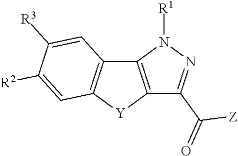

or a pharmaceutically acceptable salts thereof.

In some embodiments, R¹ is C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, Y is —OC(R⁴)₂—.

In some embodiments, Z is —OR⁴, —N(R⁴)₂, —SR⁴, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵.

In some embodiments, R² is —CF₃, —OCF₃, or —OCH₂CH₃.

In some embodiments. R³ is hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, each R⁴ is independently hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, each R⁵ is independently deuterium, halogen, —OH, —NO₂, —CN, —SR⁶, —S(═O)R⁶, —S(═O)₂R⁶, —N(R⁶)₂, —C(═O)R⁶, —OC(═O)R⁶, —C(═O)OR⁶, —C(═O)N(R⁶)₂, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₃-C₇ cycloalkyl, substituted or unsubstituted C₂-C₇ heterocycloalkyl, substituted or unsubstituted aryl, and substituted or unsubstituted heteroaryl.

In some embodiments, each R⁶ is independently hydrogen, deuterium, substituted or unsubstituted C₁-C₄ alkyl, —CD₃, substituted or unsubstituted C₁-C₄haloalkyl, substituted or unsubstituted C₁-C₄ heteroalkyl, substituted or unsubstituted C₃-C₆ cycloalkyl, substituted or unsubstituted C₂-C₅ heterocycloalkyl, substituted or unsubstituted aryl, or substituted or unsubstituted heteroaryl.

In some embodiments, Y is —O—, —S—, —NR⁴—, —SC(R⁴)₂—, —C(R⁴)₂O—, —C(R⁴)₂S—, —C(R⁴)₂NR⁴—, —C(R⁴)₂—, —S(═O)C(R⁴)₂—, —C(R⁴)₂S(═O)—, —S(═O)₂C(R⁴)₂—, —C(R⁴)₂S(═O)₂—, or —CR⁴=CR⁴—.

In some embodiments, Z is —OR⁴, —N(R⁴)₂, —SR⁴, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵.

In some embodiments, R² is —R, halogen, -haloalkyl, —OR, —SR, —CN, —NO₂, —CF₃, —OCF₃, —SO₂R, —SOR, —C(O)R, —CO₂R, —C(O)N(R)₂, —NRC(O)R, —NRC(O)N(R)₂, —NRSO₂R, or —N(R)₂;

In some embodiments, Y is —O—, —S—, —NR⁴—, —OC(R⁴)₂—, —SC(R⁴)₂—, —C(R⁴)₂O—, —C(R⁴)₂S—, —C(R⁴)₂NR⁴—, —C(R⁴)₂—, —C(R⁴)₂—C(R⁴)₂— or —CR⁴=CR⁴—.

In some embodiments, R is hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, Z is —O-t-butyl.

In some embodiments, R³ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, R³ is C₃-C₁₆ heteroaryl unsubstituted or substituted with 1, 2, 3, 4, or 5 groups selected from R⁵.

In some embodiments, R³ is selected from

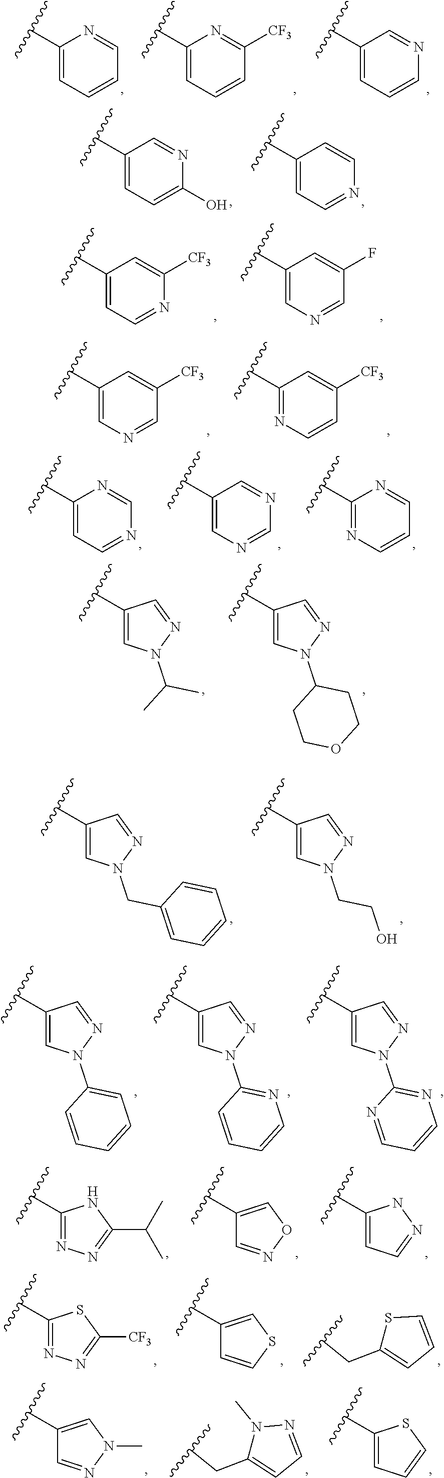

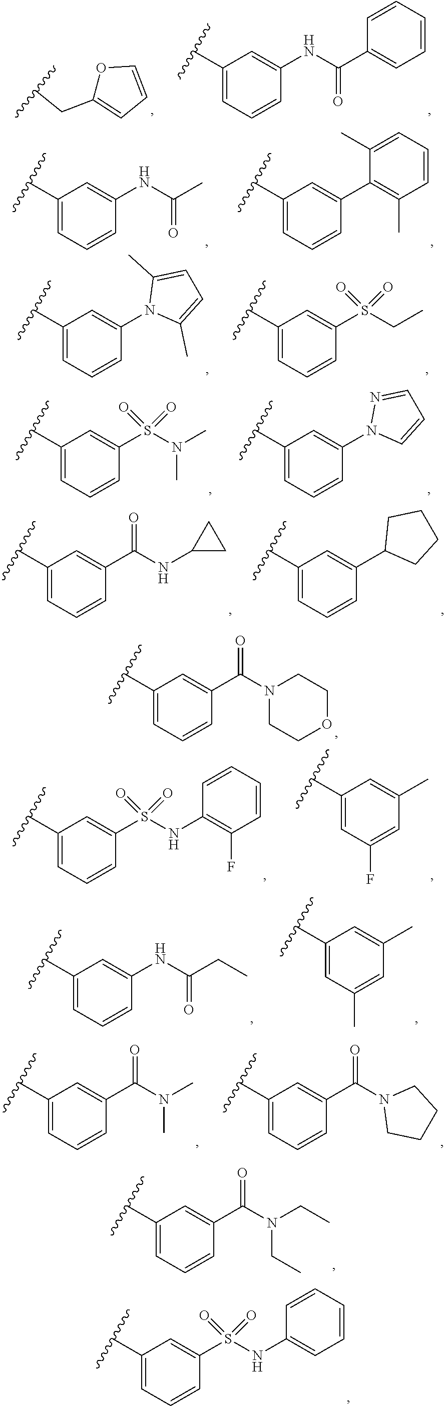

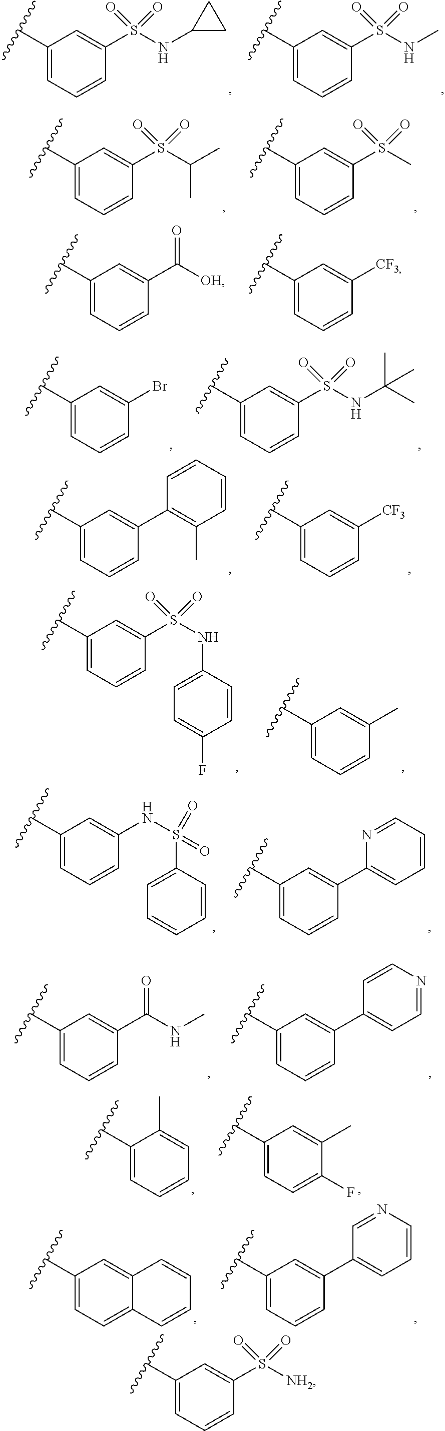

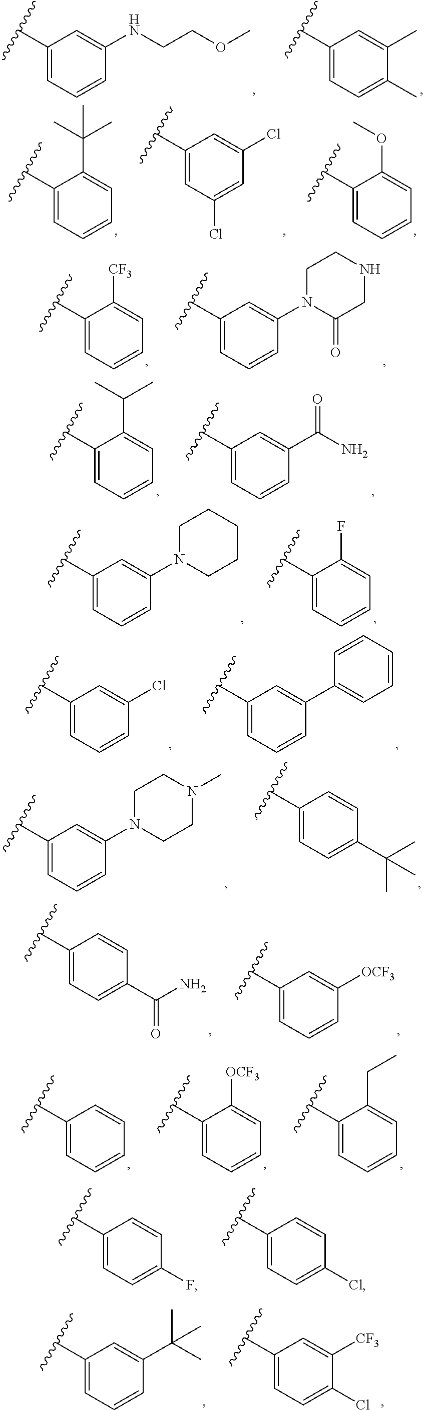

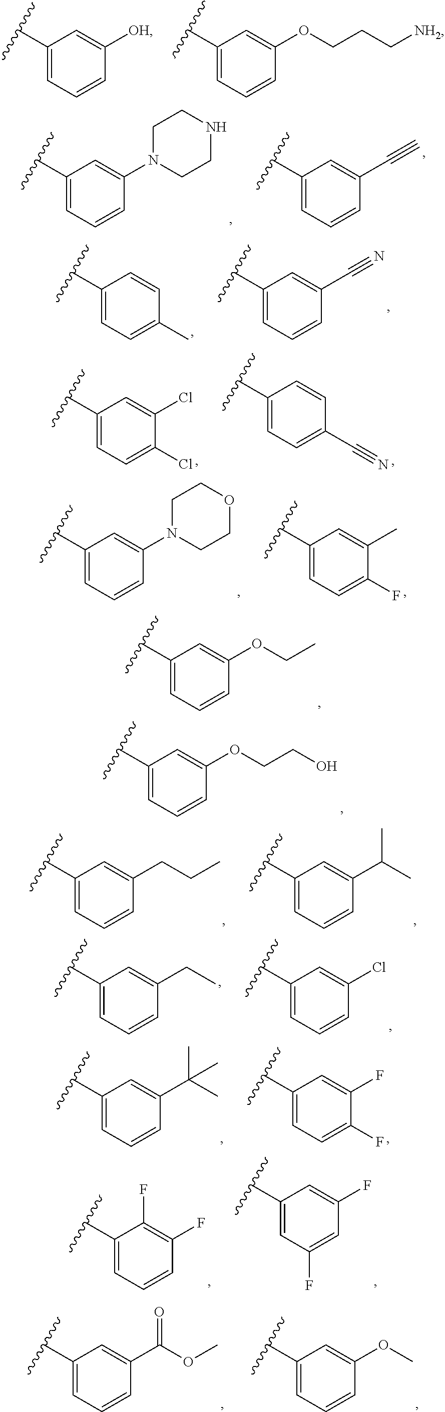

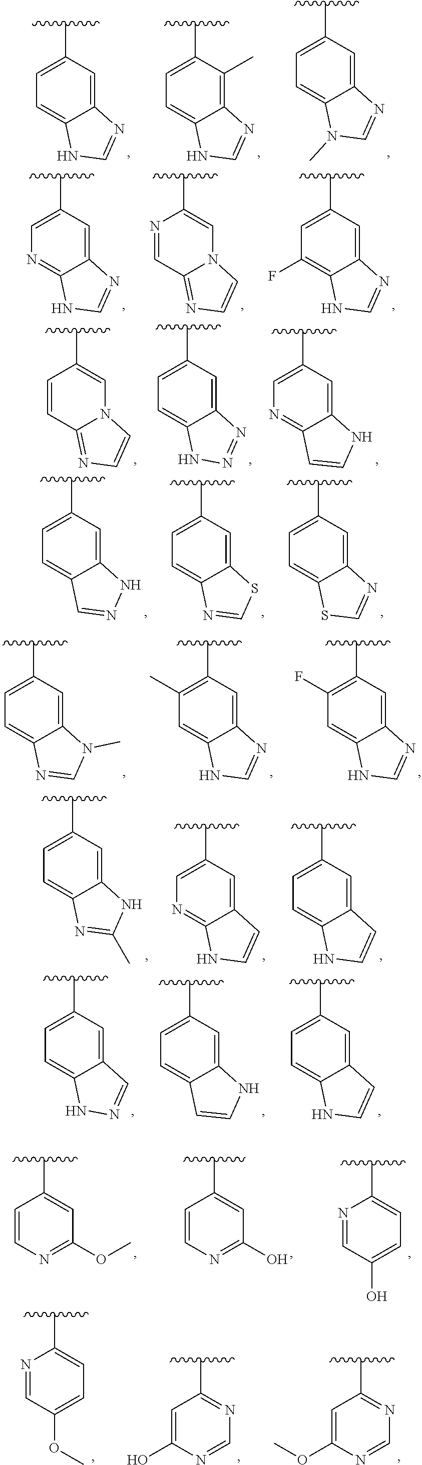

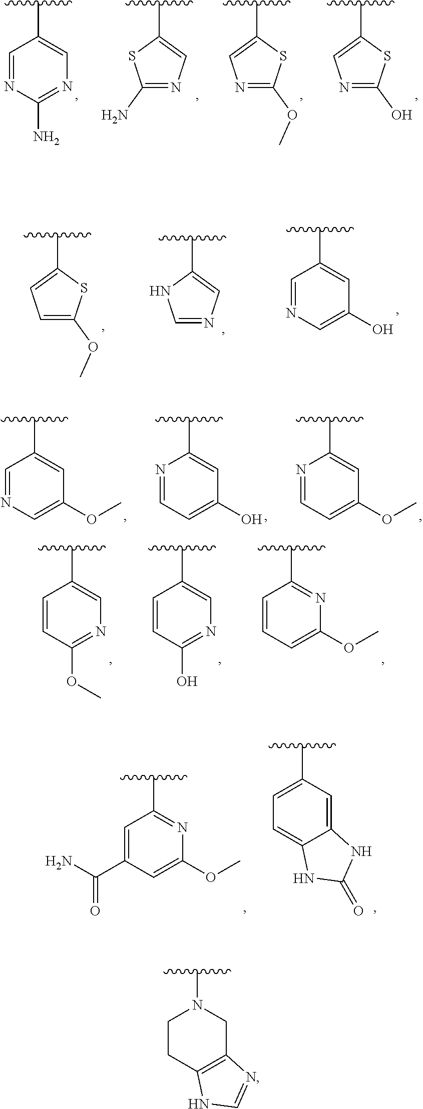

and substitutions thereof.

In some embodiments, R³ is selected from

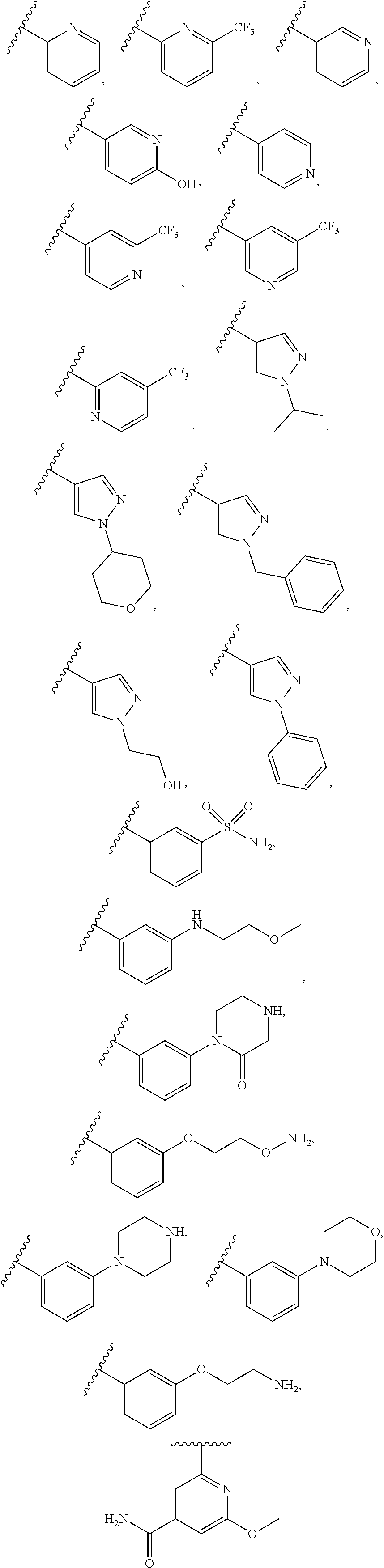

and substitutions thereof.

In some embodiments, R³ is selected from

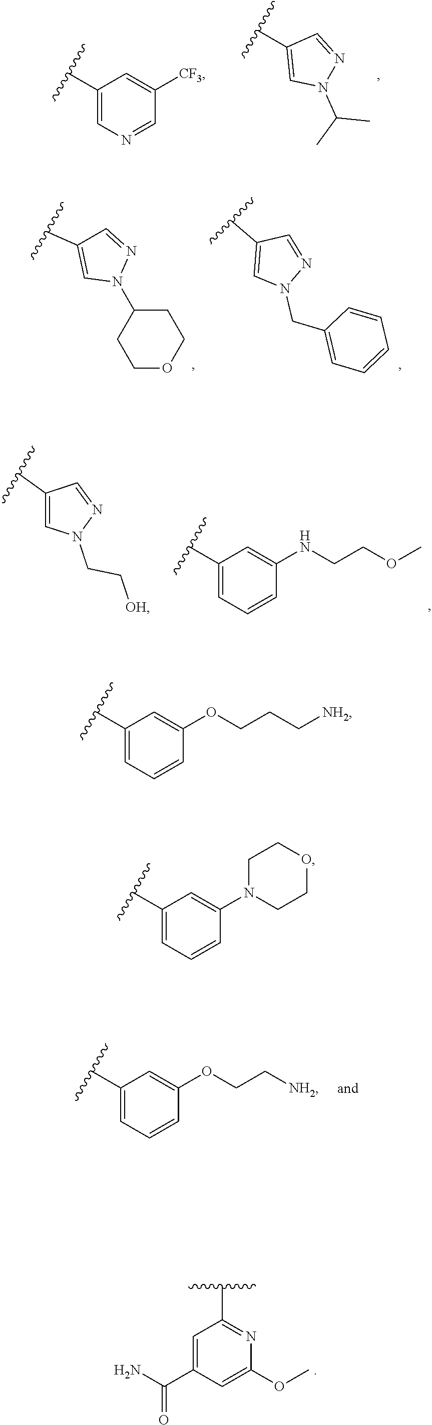

In some embodiments, R³ is

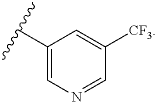 [SMILES: *c1cncc(C(F)(F)F)c1]

In some embodiments, R³ is

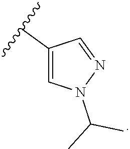 [SMILES: *c1cnn(C(C)C)c1]

In some embodiments, R³ is

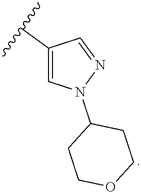 [SMILES: *c1cnn(C2CCOCC2)c1]

In some embodiments, R³ is

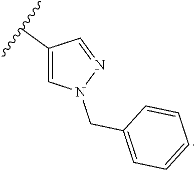 [SMILES: *c1cnn(Cc2ccccc2)c1]

In some embodiments, R³ is

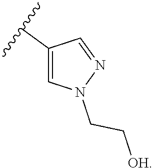 [SMILES: *c1cnn(CCO)c1]

In some embodiments, R³ is

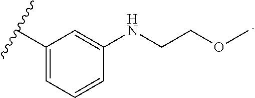 [SMILES: *c1cccc(NCCOC)c1]

In some embodiments, R³ is

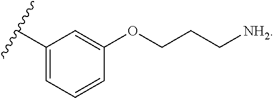 [SMILES: *c1cccc(OCCCN)c1]

In some embodiments, R³ is

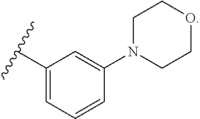 [SMILES: *c1cccc(N2CCOCC2)c1]

In some embodiments, R³ is

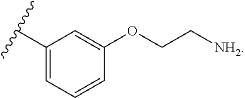 [SMILES: *c1cccc(OCCN)c1]

In some embodiments, R³ is

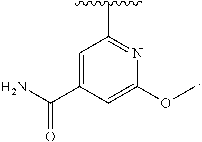 [SMILES: *c1cc(C(N)=O)cc(OC)n1]

In some embodiments, R² is -halogen, —OR, —SR, —CN, —NO₂, —CF₃, —OCF₃, or —C(═O)CH₃.

In some embodiments, R² is —OCH₃, —SCH₃, —CN, —NO₂, —CF₃, or —OCF₃.

In some embodiments, R² is —OCH₃, —SCH₃, or —OCF₃.

In some embodiments, R² is —SCH₃.

In some embodiments, R² is —OCF₃.

In some embodiments, R² is —CF₃.

In some embodiments, R² is —OCH₂CH₃.

In some embodiments, R² is —OCH₃.

In some embodiments, R¹ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, R¹ is C₆ aryl unsubstituted or substituted with 1, 2, 3, 4, or 5 groups selected from R⁵.

In some embodiments, R¹ is C₃-C₁₆ heteroaryl unsubstituted or substituted with 1, 2, 3, 4, or 5 groups selected from R⁵.

In some embodiments, R¹ is selected from

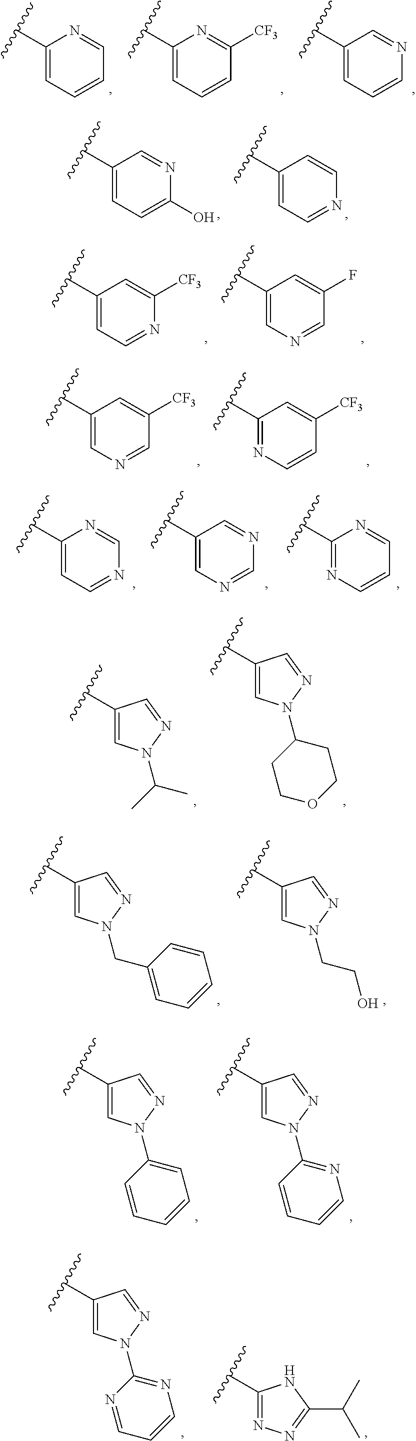

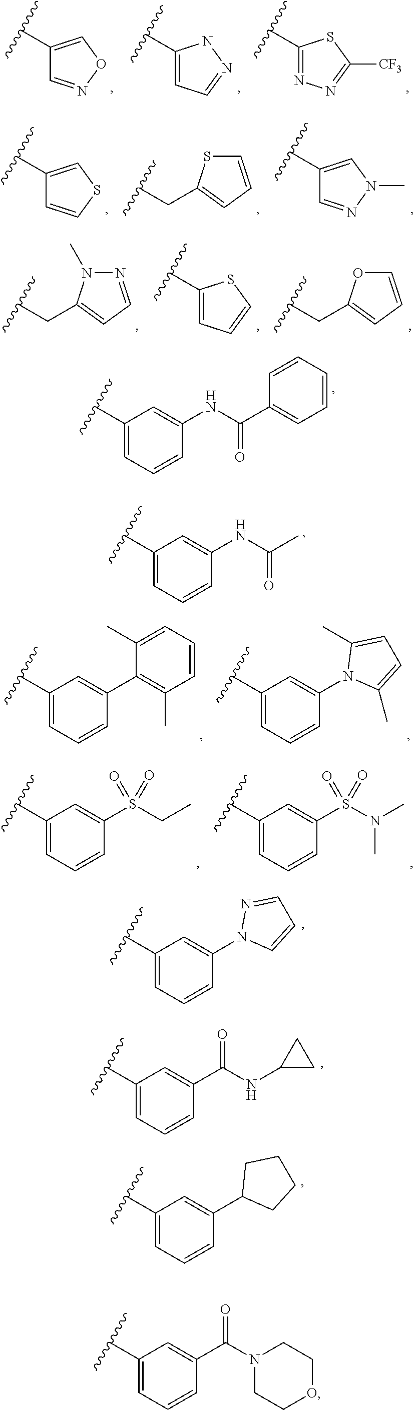

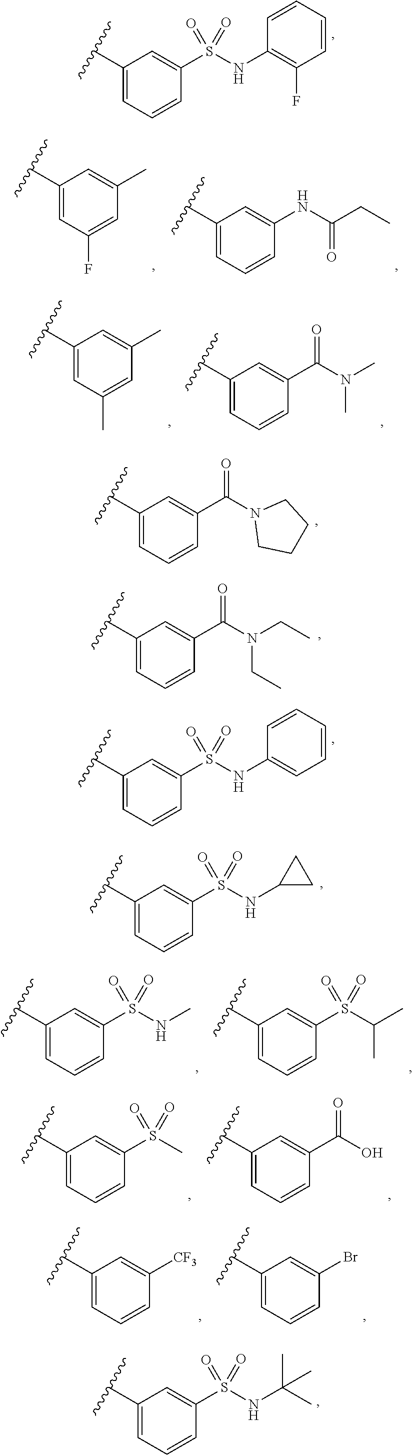

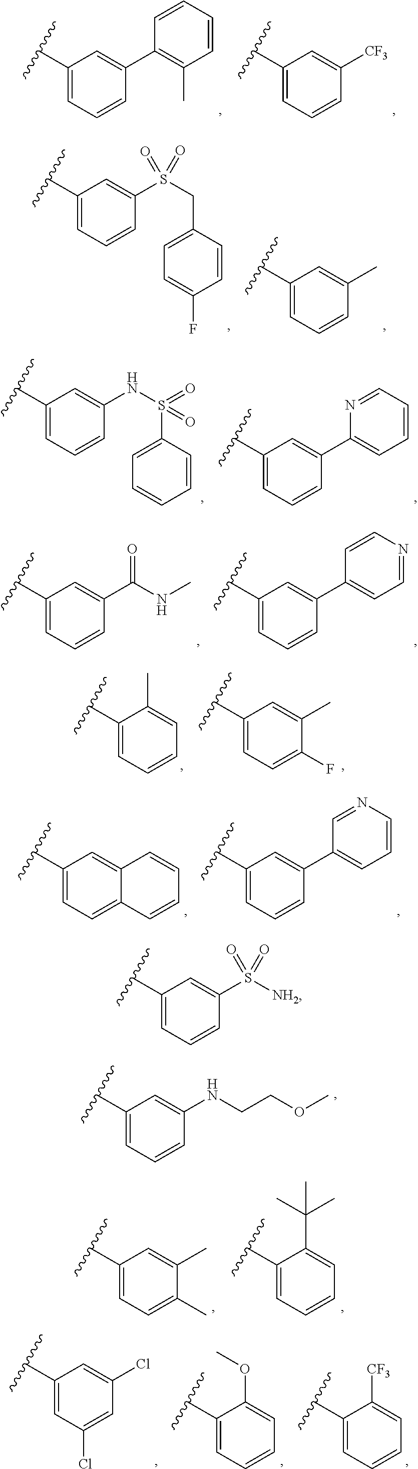

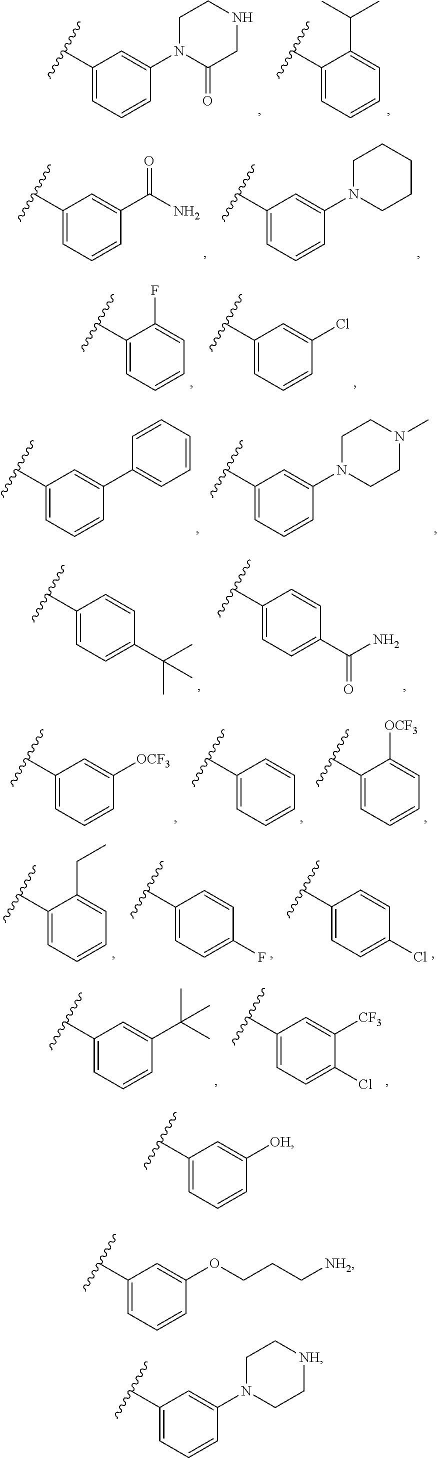

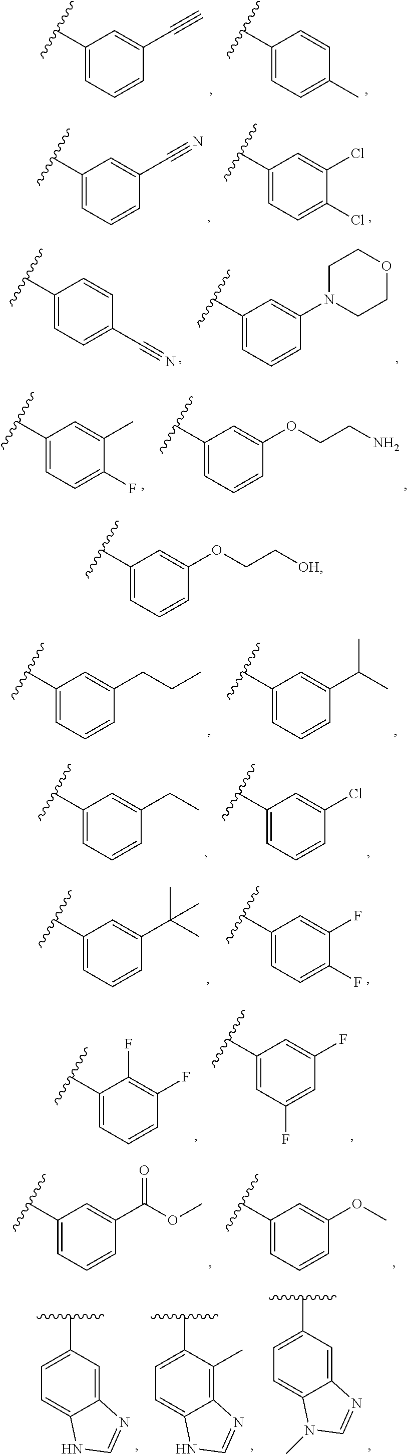

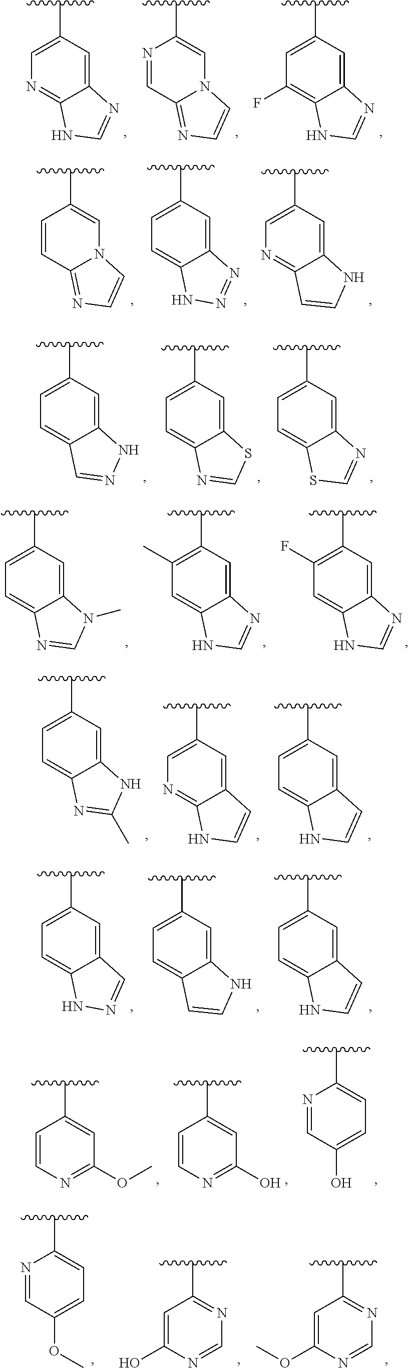

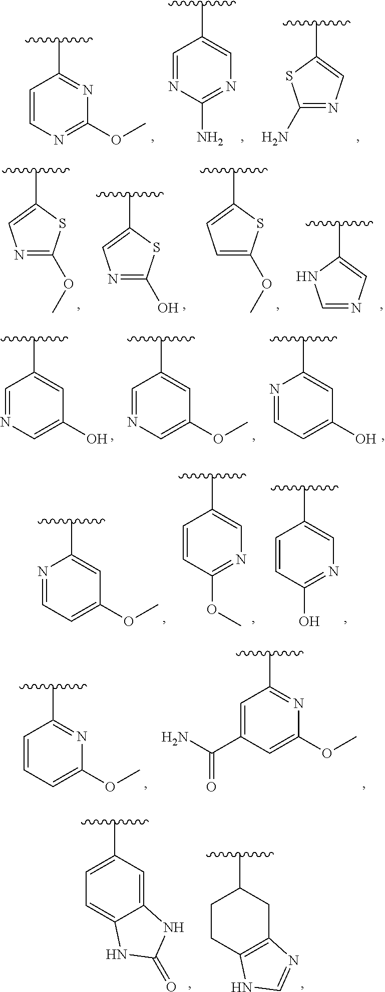

and substitutions thereof.

In some embodiments, R¹ is selected from

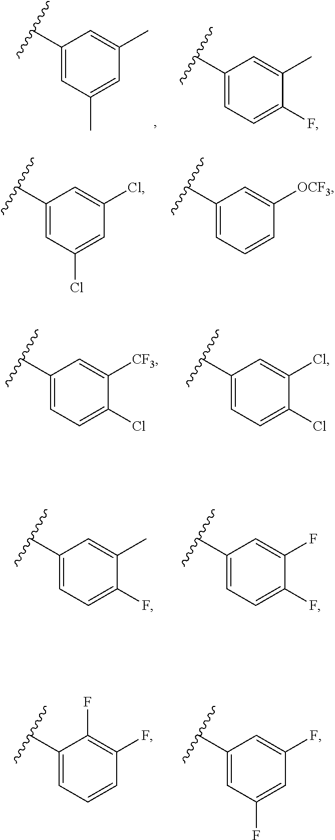

and substitutions thereof.

In some embodiments, R¹ is

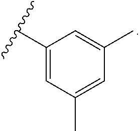 [SMILES: *c1cc(C)cc(C)c1]

In some embodiments, R¹ is

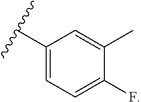 [SMILES: *c1ccc(F)c(C)c1]

In some embodiments, R¹ is

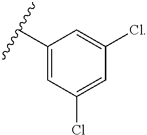 [SMILES: *c1cc(Cl)cc(Cl)c1]

In some embodiments, R¹ is

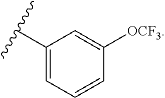 [SMILES: *c1cccc(OC(F)(F)F)c1]

In some embodiments wherein R¹ is

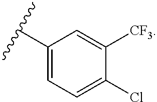 [SMILES: *c1ccc(Cl)c(C(F)(F)F)c1]

In some embodiments, R¹ is

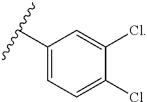 [SMILES: *c1ccc(Cl)c(Cl)c1]

In some embodiments, R¹ is

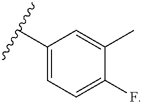 [SMILES: *c1ccc(F)c(C)c1]

In some embodiments, R¹ is

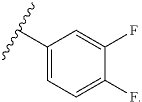 [SMILES: *c1ccc(F)c(F)c1]

In some embodiments, R¹ is

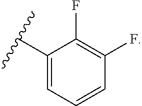 [SMILES: *c1cccc(F)c1F]

In some embodiments, R¹ is

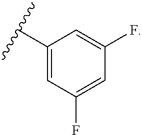 [SMILES: *c1cc(F)cc(F)c1]

In some embodiments, Y is —O—, —S—, —NR⁴—, —OC(R⁴)₂—, —SC(R⁴)₂—, —C(R⁴)₂O—, —C(R⁴)₂S—, —C(R⁴)₂NR⁴—, —C(R⁴)₂—, or —CR⁴=CR⁴—;

In some embodiments, Y is —O—, —S—, —NR⁴—, —OC(R⁴)₂—, —SC(R⁴)₂—, —C(R⁴)₂O—, —C(R⁴)₂S—, —C(R⁴)₂—.

In some embodiments, Y is —O—, —S—, —NH—, —OCH₂—, —SCH₂—, —CH₂O—, —CH₂S—, —CH₂—.

In some embodiments, Y is —O—.

In some embodiments, Y is —S—.

In some embodiments, Y is —S(═O)—.

In some embodiments, Y is —S(═O)₂—.

In some embodiments, Y is —S(═O)C(R⁴)₂—.

In some embodiments, Y is —C(R⁴)₂S(═O)—.

In some embodiments, Y is —S(═O)₂C(R⁴)₂—.

In some embodiments, Y is —C(R⁴)₂S(═O)₂—.

In some embodiments, Y is —S(═O)CH₂—.

In some embodiments, Y is —CH₂S(═O)—.

In some embodiments, Y is —S(═O)₂CH₂—.

In some embodiments, Y is —CH₂S(═O)₂—.

In some embodiments, Y is —NR⁴—.

In some embodiments, Y is —OC(R⁴)₂—.

In some embodiments, Y is —SC(R⁴)₂—.

In some embodiments, Y is —C(R⁴)₂O—.

In some embodiments, Y is —C(R⁴)₂S—.

In some embodiments, Y is—C(R⁴)₂NR⁴—.

In some embodiments, Y is —C(R⁴)₂—.

In some embodiments, Y is —C(R⁴)₂—C(R⁴)₂—.

In some embodiments, Y is —CR⁴=CR⁴—;

In some embodiments, Y is —NH—. In some embodiments, Y is —OCH₂—. In some embodiments, Y is —SCH₂—. In some embodiments, Y is —CH₂O—. In some embodiments, Y is —CH₂S—. In some embodiments, Y is—CH₂NR⁴—. In some embodiments, Y is —CH₂—. In some embodiments, Y is —CH₂—CH₂—. In some embodiments, Y is —CH═CH—.

In some embodiments, Z is —OR⁴, —N(R⁴)₂, —SR⁴, —CF₃, —OCF₃, —OH, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, Z is —OR⁴, —N(R⁴)₂, —SR⁴, —CF₃, —OCF₃, or is selected from

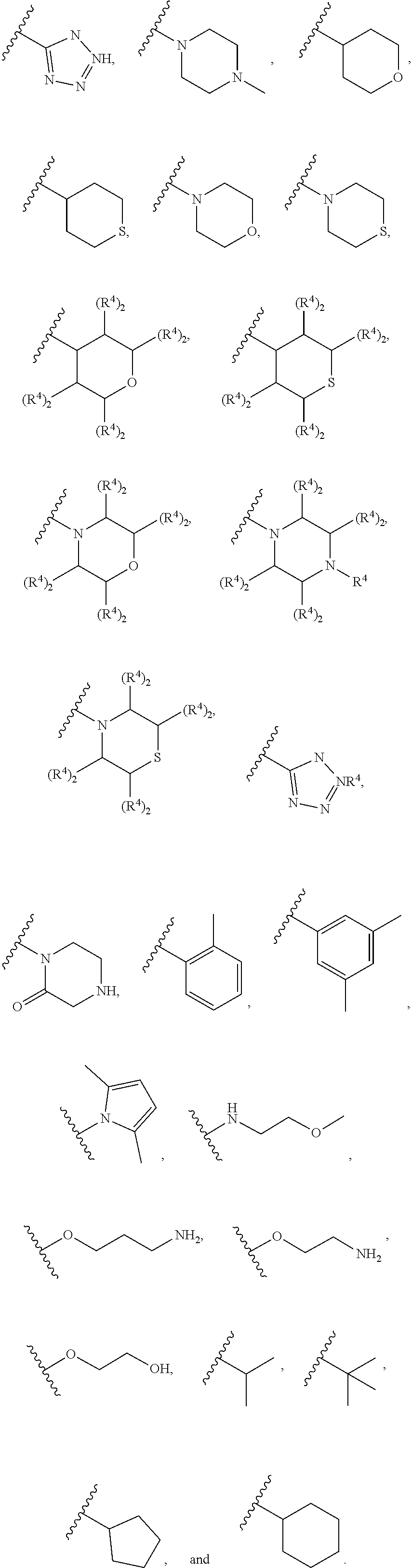

In some embodiments, Z is —OR⁴, —N(R⁴)₂, —SR⁴.

In some embodiments, Z is —OR⁴, —N(R⁴)₂, —SR⁴, and at least one R⁴ of Z is selected from

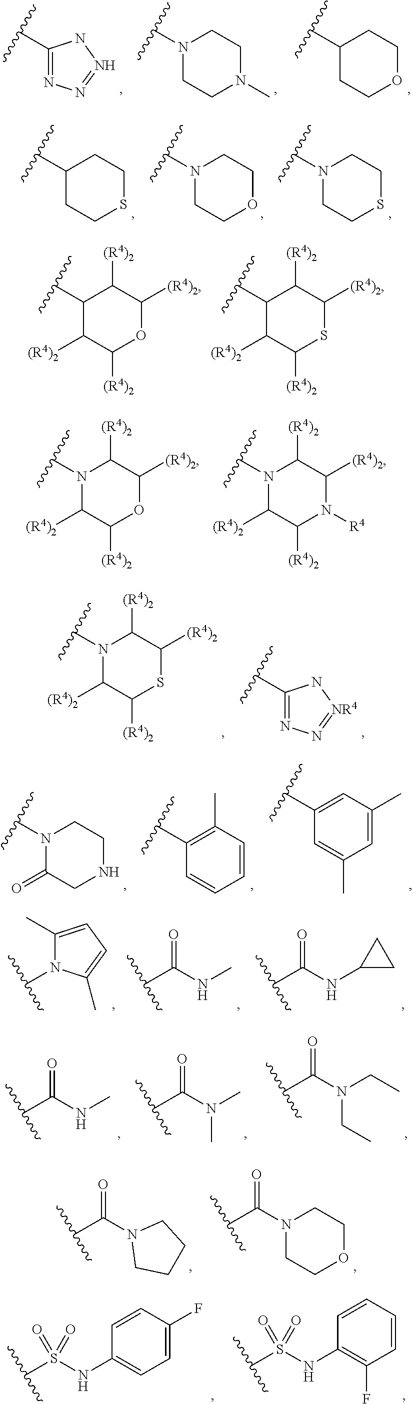

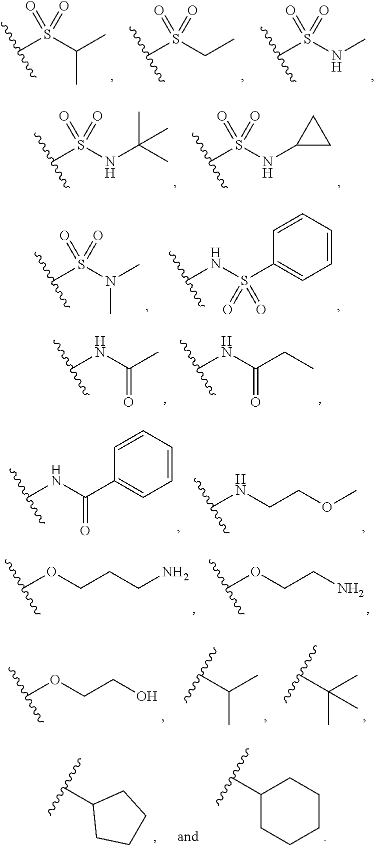

In some embodiments, Z is —OR⁴ or —SR⁴, and the R⁴ of Z is

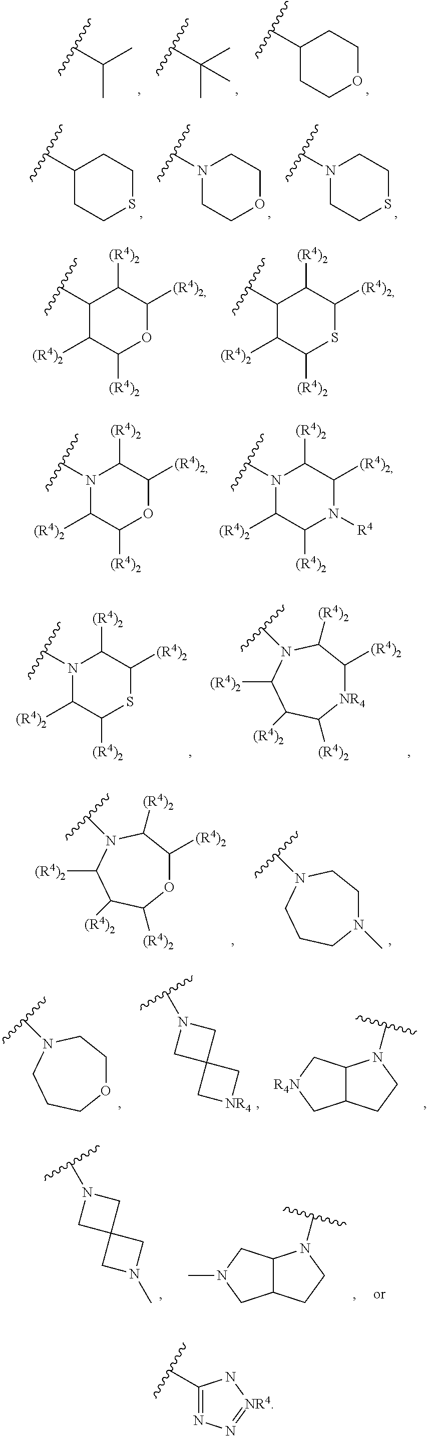

In some embodiments, Z selected from N

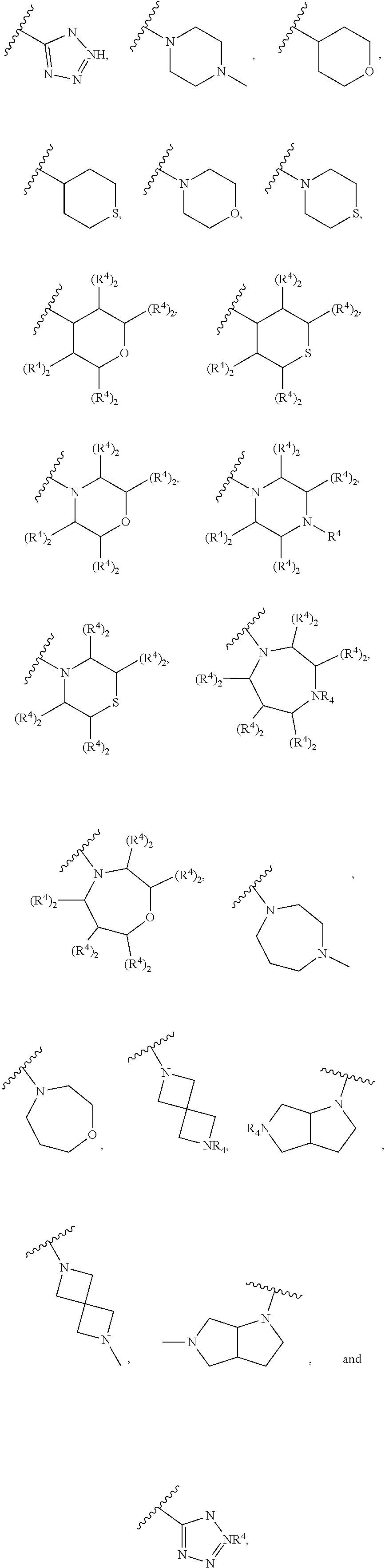

In some embodiments, Z is

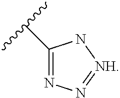

In some embodiments, Z is N

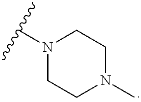 [SMILES: *N1CCN(C)CC1]

In some embodiments, Z is

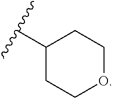 [SMILES: *C1CCOCC1]

In some embodiments, Z is

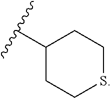 [SMILES: *C1CCSCC1]

In some embodiments, Z is

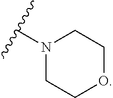 [SMILES: *N1CCOCC1]

In some embodiments, Z is

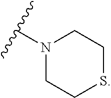 [SMILES: *N1CCSCC1]

In some embodiments, Z is

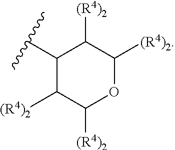

In some embodiments, Z is

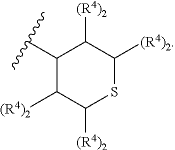

In some embodiments, Z is

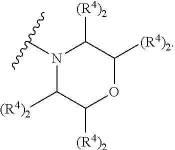

In some embodiments, Z is

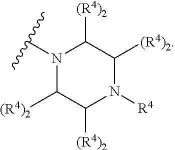

In some embodiments, Z is

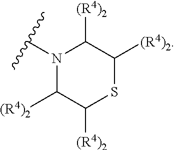

In some embodiments, Z is

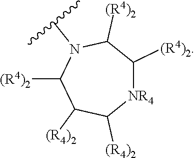

In some embodiments, Z is

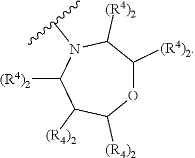

In some embodiments, Z is

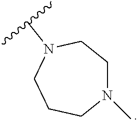 [SMILES: *N1CCCN(C)CC1]

In some embodiments, Z is

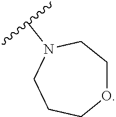 [SMILES: *N1CCCOCC1]

In some embodiments, Z is

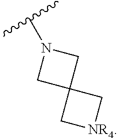

In some embodiments, Z is

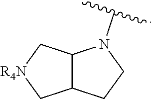

In some embodiments, Z is

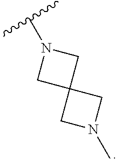 [SMILES: *N1CC2(C1)CN(C)C2]

In some embodiments, Z is

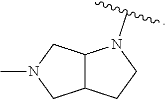 [SMILES: *N1CCC2CN(C)CC21]

In some embodiments, Z is

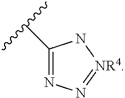

In some embodiments, Z is

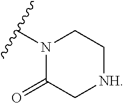 [SMILES: *N1CCNCC1=O]

In some embodiments, Z is

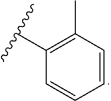 [SMILES: *c1ccccc1C]

In some embodiments, Z is

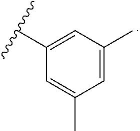 [SMILES: *c1cc(C)cc(C)c1]

In some embodiments, Z is

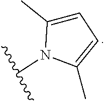 [SMILES: *n1c(C)ccc1C]

In some embodiments, Z is

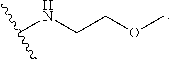 [SMILES: *NCCOC]

In some embodiments, Z is

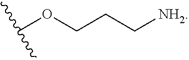 [SMILES: *OCCCN]

In some embodiments, Z is

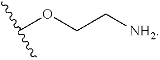 [SMILES: *OCCN]

In some embodiments, Z is

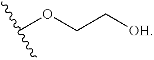 [SMILES: *OCCO]

In some embodiments, Z is

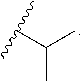 [SMILES: *C(C)C]

In some embodiments, Z is

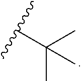 [SMILES: *C(C)(C)C]

In some embodiments, Z is

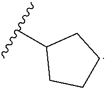 [SMILES: *C1CCCC1]

In some embodiments, Z is

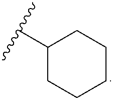 [SMILES: *C1CCCCC1]

In some embodiments, Z is

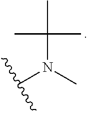 [SMILES: *N(C)C(C)(C)C]

In some embodiments, Z is

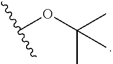 [SMILES: *OC(C)(C)C]

In some embodiments, R¹ or R³ is substituted with halogen.

In some embodiments, R¹ or R³ is substituted with chlorine.

In some embodiments, R¹ or R³ is substituted with fluorine.

In some embodiments, R¹ or R³ is substituted with C₁-C₄ heteroalkyl.

In some embodiments, at least one R⁴ within Z is selected from

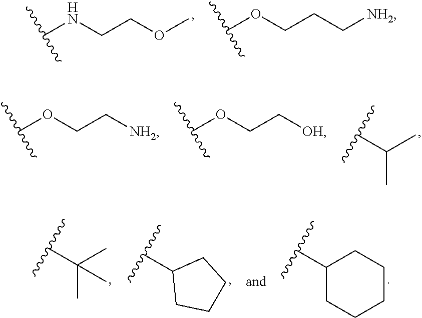

In some embodiments, FSH Modulators disclosed herein have a structure selected from the group of Compound 1-01, Compound 1-02A, Compound 1-02, Compound 1-03, Compound 1-04, Compound 1-05, Compound 1-06, Compound 2-01, Compound 2-02, Compound 2-03, Compound 2-04, Compound 2-05, Compound 2-06, Compound 2-07, Compound 2-08, Compound 3-01, Compound 3-02, Compound 3-03, Compound 3-04, Compound 3-07, Compound 3-08, Compound 3-09, Compound 3-10A, Compound 3-10, Compound 3-11, Compound 3-12, Compound 4-01A, Compound 4-01, Compound 4-02A, Compound 4-02, Compound 4-03A, Compound 4-03, Compound 4-04A,Compound 4-04, Compound 4-05A, Compound 4-05, Compound 4-06A, Compound 4-06, Compound 4-07A, Compound 4-07, Compound 4-08A, Compound 4-08, Compound 5-01, Compound 5-02, Compound 5-03, Compound 5-04, Compound 5-05, Compound 5-06, Compound 5-07, Compound 5-08, Compound 6-01A, Compound 6-01B, Compound 6-01, Compound 6-02A, Compound 6-02B, Compound 6-02, Compound 6-03, Compound 6-04, Compound 6-05, Compound 6-06, Compound 6-07, Compound 6-08, Compound 8-01, Compound 8-02, Compound 8-03, Compound 8-05, Compound 8-06, Compound 8-07A, Compound 8-07, Compound 8-09, Compound 8-10, Compound 8-14, Compound 8-15, Compound 8-16B, Compound 8-16, Compound 8-17, Compound 8-20, Compound 8-21, Compound 8-22, Compound 8-23, Compound 8-24, Compound 8-25, Compound 8-26A, Compound 8-26, Compound 8-27, Compound 8-28, Compound 8-29, Compound 8-30, Compound 8-31, Compound 8-32, Compound 8-33, Compound 8-34, Compound 8-39, and Compound 8-44.

In some embodiments, FSH Modulators disclosed herein have a structure selected from the group of: Compound 8-77, Compound 8-75, Compound 8-76, Compound 8-78, Compound 8-81, Compound 8-61, Compound 8-60, Compound 8-63, Compound 8-58, Compound 8-51, Compound 8-67, Compound 8-74, Compound 8-4, Compound 8-8, Compound 8-4a, Compound 8-13, Compound 8-57, Compound 8-18, Compound 8-35, Compound 8-36, Compound 8-37, Compound 8-38, Compound 8-41, Compound 8-42, Compound 8-43, Compound 8-45, Compound 8-46, Compound 8-47, Compound 8-49, Compound 8-50, Compound 8-52A, Compound 8-54A, Compound 8-55, Compound 8-56, Compound 8-62, Compound 8-64, Compound 8-65, Compound 8-69, Compound 8-70, Compound 8-71, Compound 8-79, Compound 8-82, Compound 8-83, Compound 8-84, Compound 8-86, Compound 8-87, Compound 8-89, Compound 9-13, Compound 9-21, Compound 9-4, Compound 9-5, Compound 9-11, Compound 9-14, Compound 9-9, Compound 9-15, Compound 9-2, Compound 9-7, Compound 9-12, Compound 9-16, Compound 9-17, Compound 9-18, Compound 9-19, Compound 9-20, Compound 10-1, Compound 10-2, Compound 10-3, Compound 10-6, Compound 10-7, Compound 10-8, Compound 10-9, Compound 10-10, Compound 11-1A, Compound 11-2, Compound 11-1, Compound 11-3, Compound 12-2, Compound 12-23, Compound 12-13, Compound 12-15, Compound 12-16, Compound 12-1, Compound 12-4, Compound 12-18, Compound 12-19, Compound 13-1, Compound 13-4, Compound 13-9, Compound 13-7, Compound 13-8, Compound 13-2, Compound 13-5, Compound 15-1, Compound 15-3, Compound 15-4, Compound 15-5, Compound 15-9, Compound 15-2, Compound 15-6, Compound 15-10, Compound 12-05, Compound 12-07, Compound 12-11, Compound 12-12, Compound 14-03, Compound 15-08, Compound 15-10, Compound 3-05, Compound 3-06, Compound 4-03B, Compound 8-04A, Compound 8-16A, Compound 8-23A, Compound 8-25A, Compound 8-26B, Compound 8-31A, Compound 8-33A, Compound 8-44, Compound 8-66, Compound 8-72, Compound 8-90, Compound 8-90A, Compound 9-01, Compound 9-03, Compound 9-06, Compound 9-08, Compound 9-08A, Compound 9-10, Compound 9-19A, and Compound 9-24.

In another aspect, described herein are methods of modulating FSH using compounds described herein. In some embodiments, compounds described herein selectively modulate FSH and do not substantially modulate TSH. In some embodiments, the methods comprise administering a compound described herein to a subject. In some embodiments, compounds described herein are FSH agonists. In some embodiments, compounds described herein are selective by at least 3-fold for FSH over TSH (e.g. at least 3, 5, 10, 20, 50, or 100 fold). In some embodiments, an in-vitro or in-vivo EC₅₀ for FSH agonism is no more than about 100 nM (e.g. no more than 100 nM, 50 nM, 10 nM, 5 nM, 1 nM, or 500 pM).

In some embodiments, methods described herein comprise treating a disease or condition comprising administering a compound described herein to a subject in need thereof. In some embodiments, the disease or condition is a fertility disorder or male hypogonadism. In some embodiments, the disease or condition is cancer. In some embodiments, the cancer is breast, prostate, colon, pancreas, urinary bladder, kidney, lung, liver, stomach, testicular, or ovarian cancer. In some embodiments, the disease or condition is a cardiovascular condition. In some embodiments, the cardiovascular condition is atherosclerosis. In some embodiments, the disease or condition is a body composition disorder (e.g. obesity). In some embodiments, the disease or condition is non-alcoholic fatty liver disease. In some embodiments, the disease or condition is a bone density disorder (e.g. osteoporosis). In some embodiments, the disease or condition is Turner syndrome, Klinefelter syndrome, polycystic ovary syndrome (PCOS), and/or primary ovary insufficiency (POI).

In some embodiments, the disease or condition is polycystic ovary syndrome (PCOS).

In some embodiments, the disease or condition is Turner syndrome.

In some embodiments, the disease or condition is Klinefelter syndrome.

In some embodiments, the disease or condition is primary ovary insufficiency (POI).

In another aspect, described herein are pharmaceutical compositions comprising any of the compounds described herein or a pharmaceutically acceptable salt or pharmaceutically acceptable solvate thereof, and a pharmaceutically acceptable excipient or carrier.

In another aspect, described herein are pharmaceutically acceptable lipid nanoparticle formulation comprising compounds described herein.

In some embodiments, the methods comprise treating a condition or disease comprising administering a compound or a pharmaceutically acceptable salt or pharmaceutically acceptable solvate thereof described herein to a subject in need thereof.

In some embodiments the methods comprise use of a compound or a pharmaceutically acceptable salt or pharmaceutically acceptable solvate thereof described herein in the manufacture of a medicament for the treatment of a condition or disease.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the nuclear magnetic resonance of Compound 1-01.

FIG. 2 shows the nuclear magnetic resonance of Compound 1-02A.

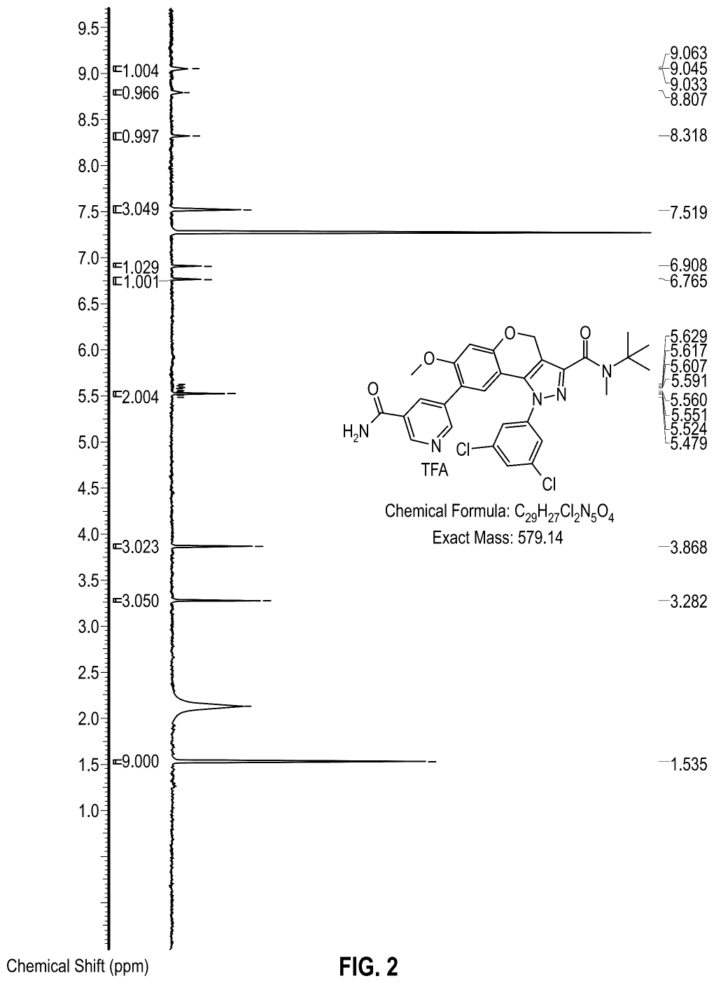

FIG. 3 shows the nuclear magnetic resonance of Compound 1-02.

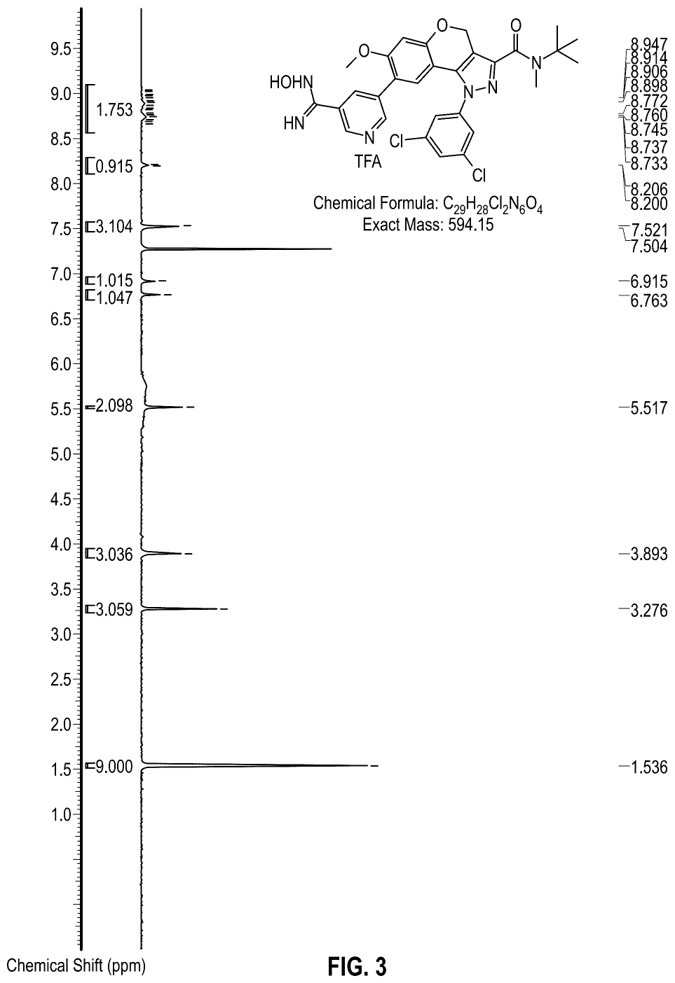

FIG. 4 shows the nuclear magnetic resonance of Compound 1-03.

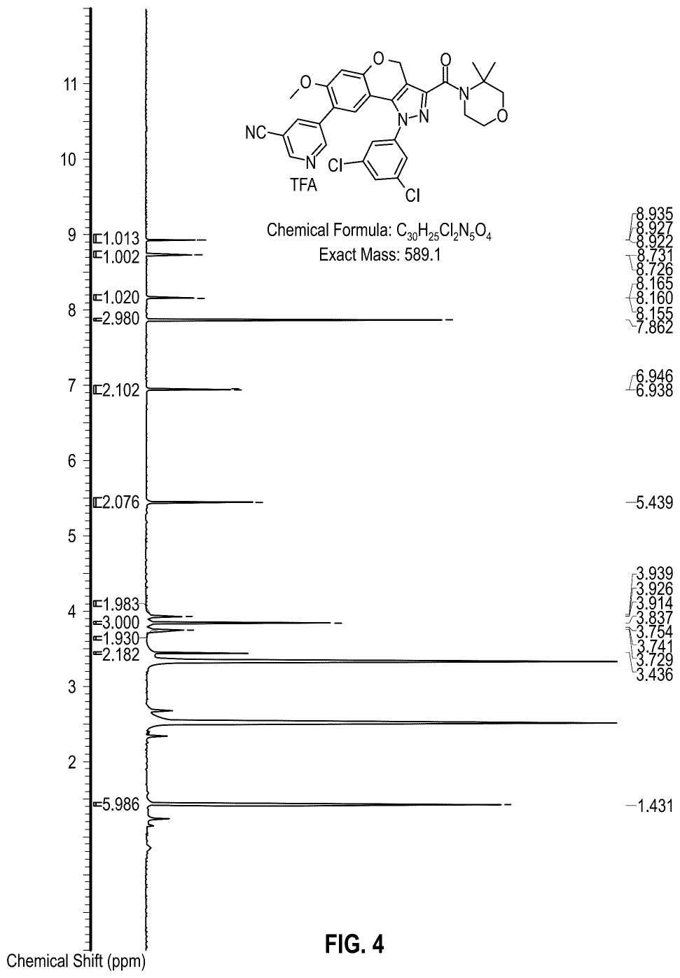

FIG. 5 shows the nuclear magnetic resonance of Compound 1-04.

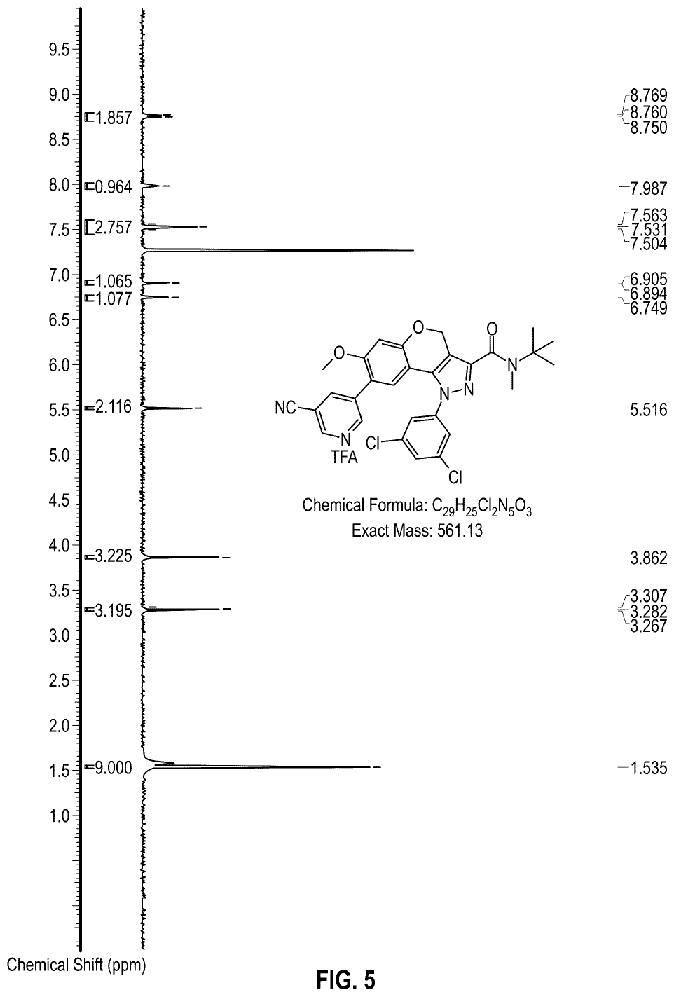

FIG. 6 shows the nuclear magnetic resonance of Compound 1-05.

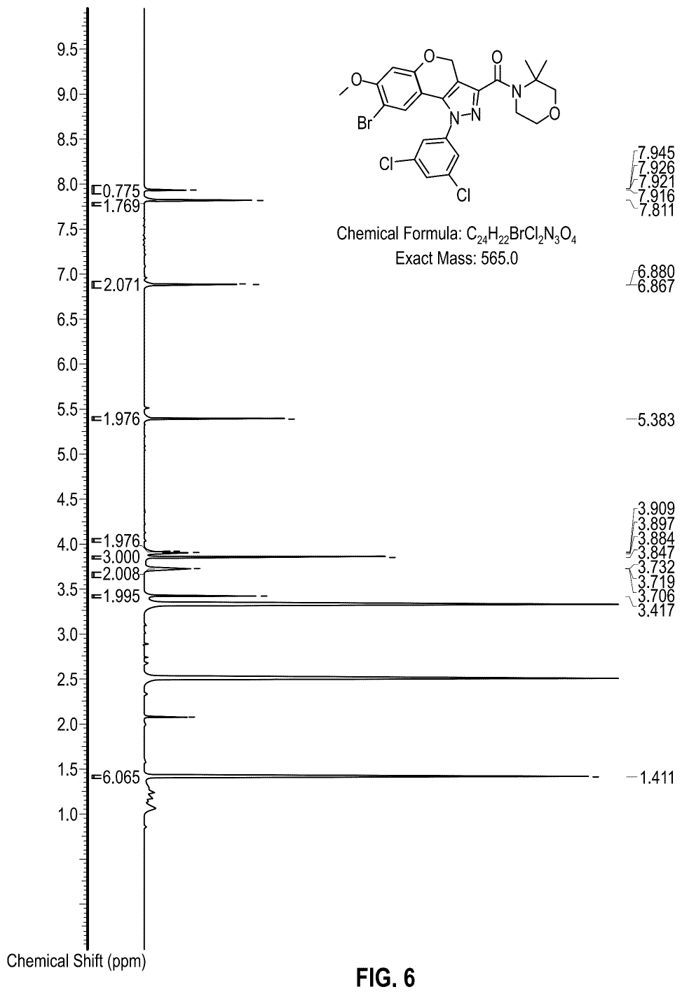

FIG. 7 shows the nuclear magnetic resonance of Compound 1-06.

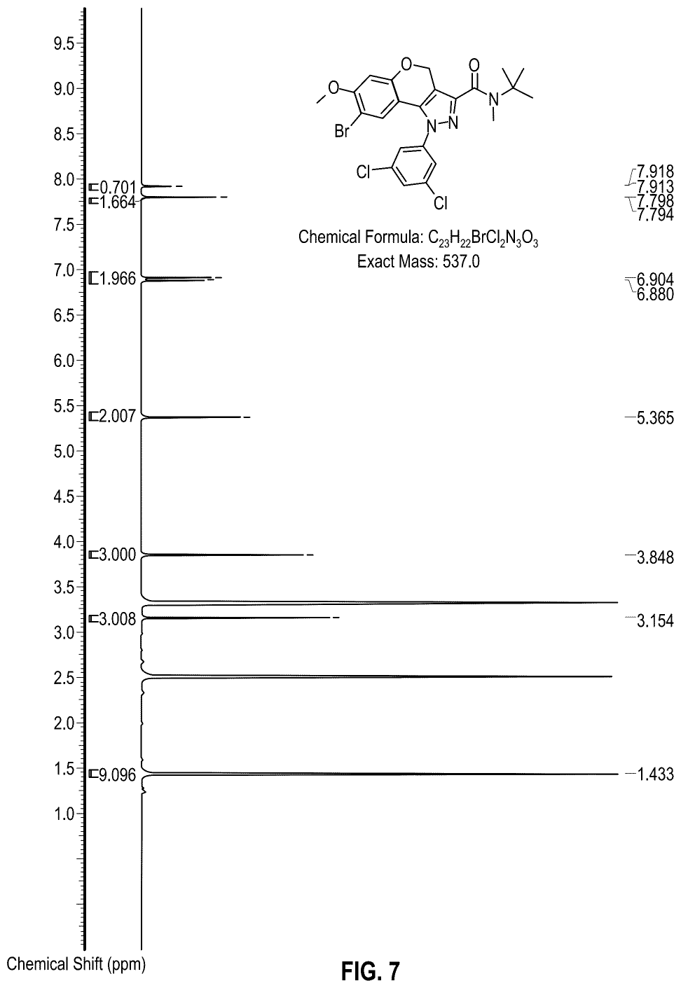

FIG. 8 shows the nuclear magnetic resonance of Compound 2-01.

FIG. 9 shows the nuclear magnetic resonance of Compound 2-02.

FIG. 10 shows the nuclear magnetic resonance of Compound 2-03.

FIG. 11 shows the nuclear magnetic resonance of Compound 2-04.

FIG. 12 shows the nuclear magnetic resonance of Compound 2-05.

FIG. 13 shows the nuclear magnetic resonance of Compound 2-06.

FIG. 14 shows the nuclear magnetic resonance of Compound 2-07.

FIG. 15 shows the nuclear magnetic resonance of Compound 2-08.

FIG. 16 shows the nuclear magnetic resonance of Compound 3-01.

FIG. 17 shows the nuclear magnetic resonance of Compound 3-02.

FIG. 18 shows the nuclear magnetic resonance of Compound 3-03.

FIG. 19 shows the nuclear magnetic resonance of Compound 3-04.

FIG. 20 shows the nuclear magnetic resonance of Compound 3-07.

FIG. 21 shows the nuclear magnetic resonance of Compound 3-08.

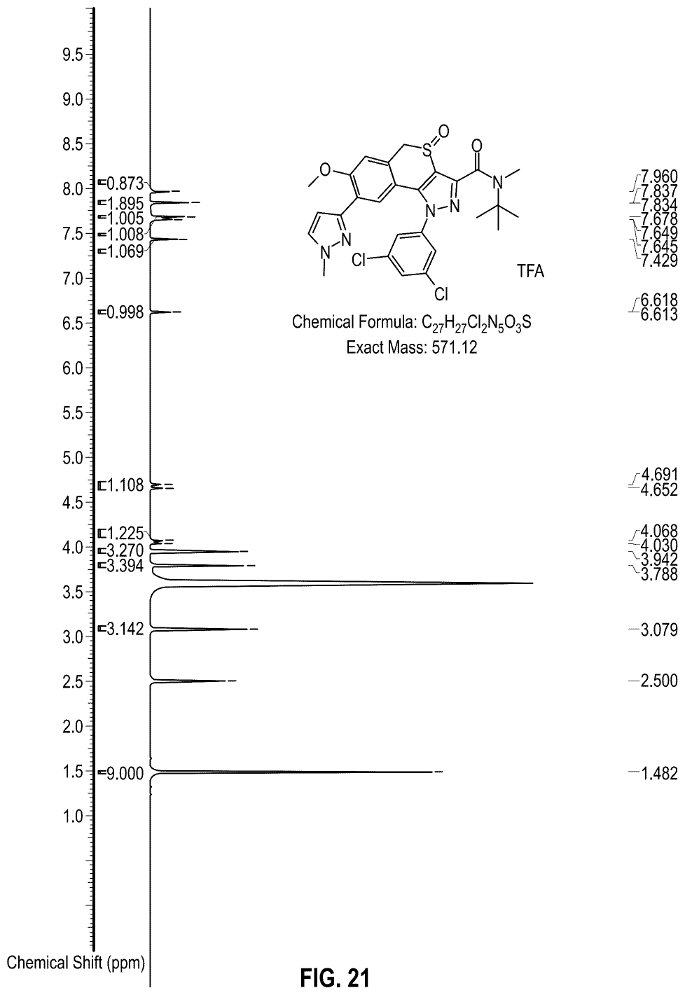

FIG. 22 shows the nuclear magnetic resonance of Compound 3-09.

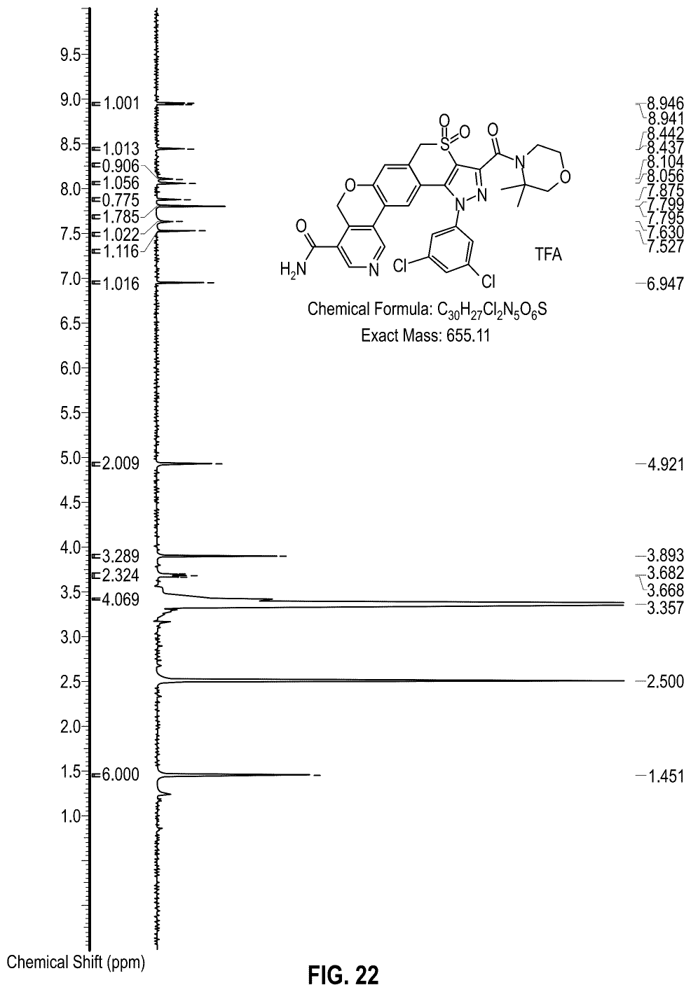

FIG. 23 shows the nuclear magnetic resonance of Compound 3-10A.

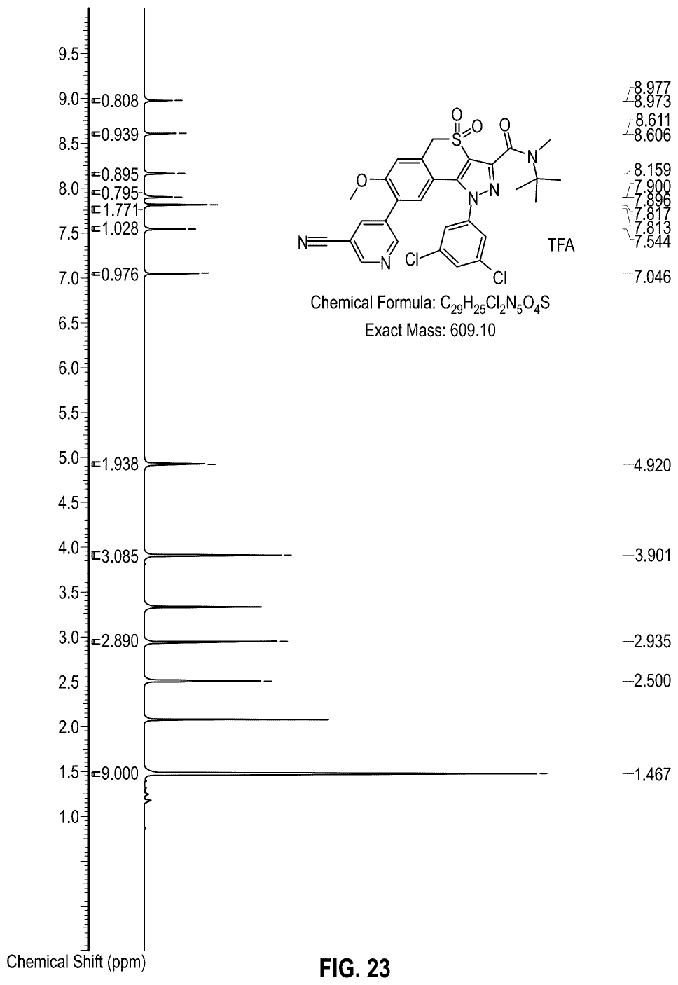

FIG. 24 shows the nuclear magnetic resonance of Compound 3-10.

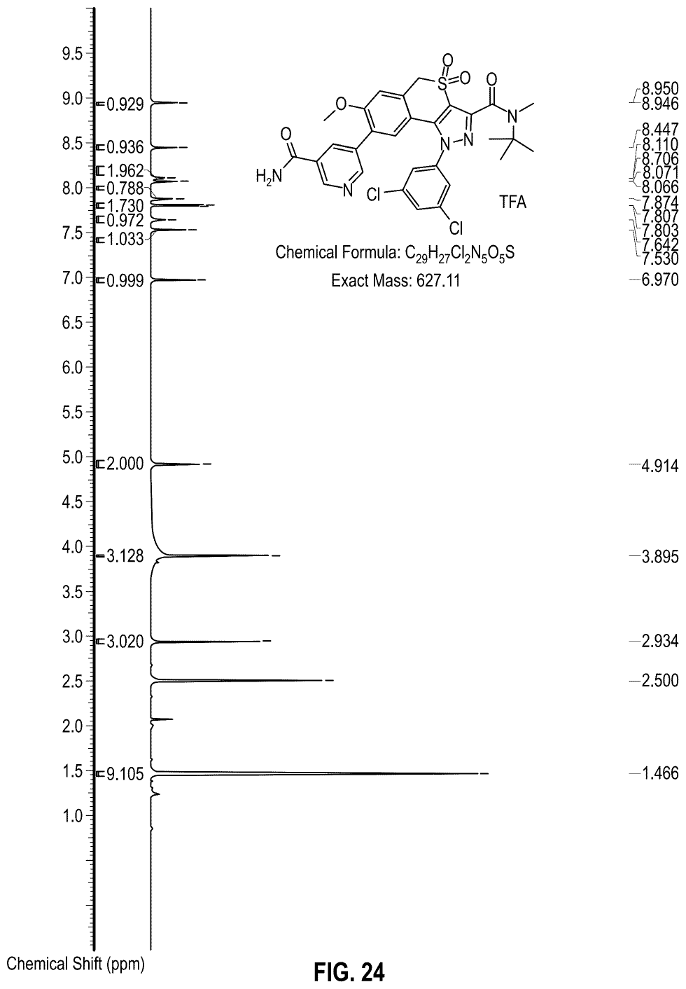

FIG. 25 shows the nuclear magnetic resonance of Compound 3-11.

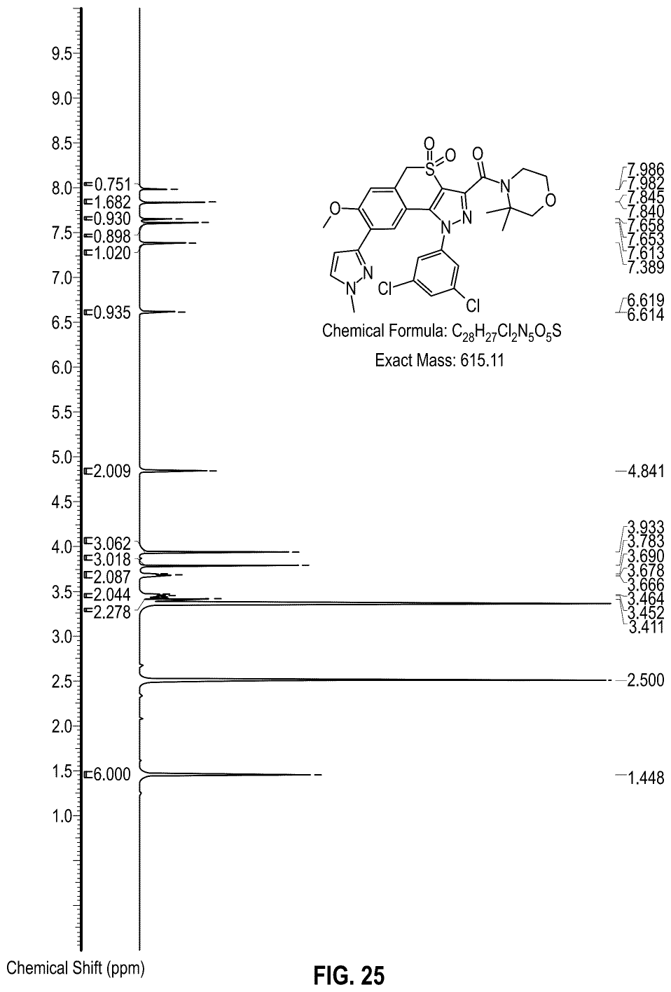

FIG. 26 shows the nuclear magnetic resonance of Compound 3-12.

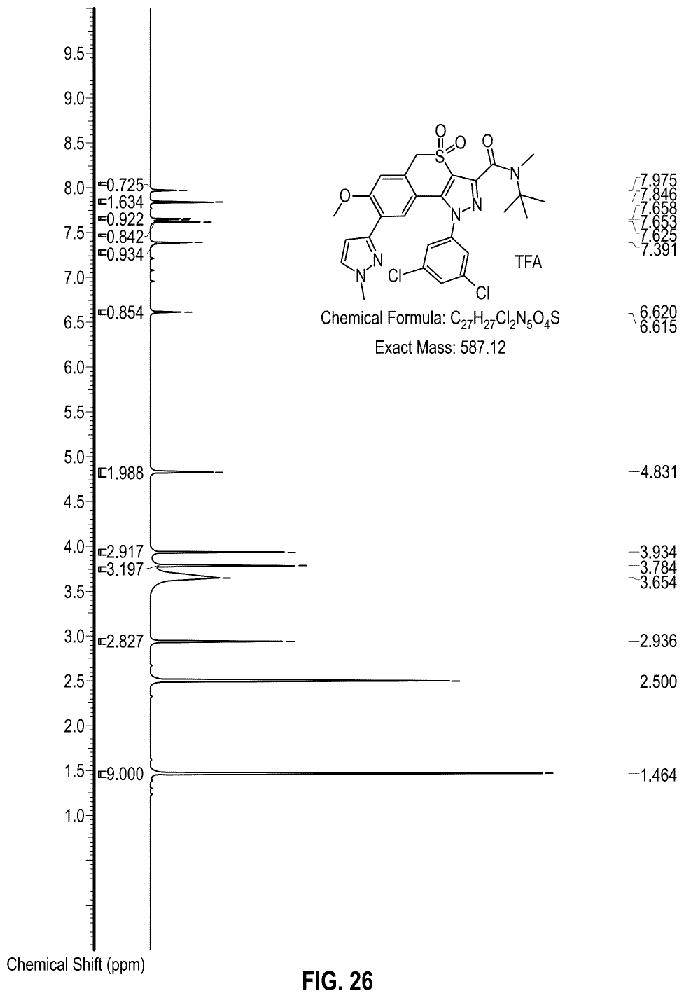

FIG. 27 shows the nuclear magnetic resonance of Compound 4-01A.

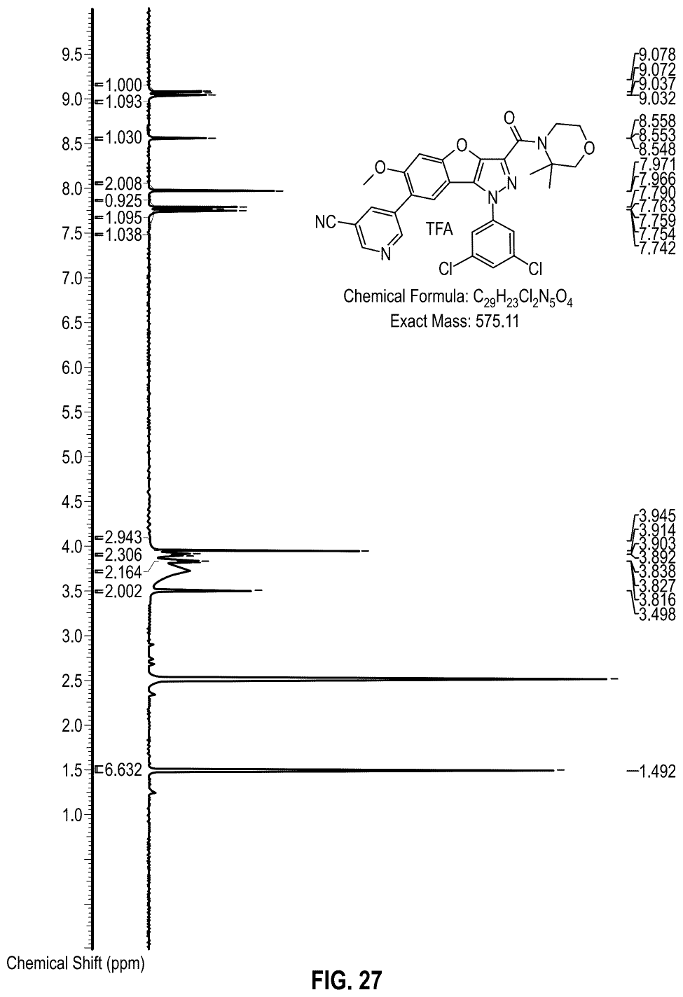

FIG. 28 shows the nuclear magnetic resonance of Compound 4-01.

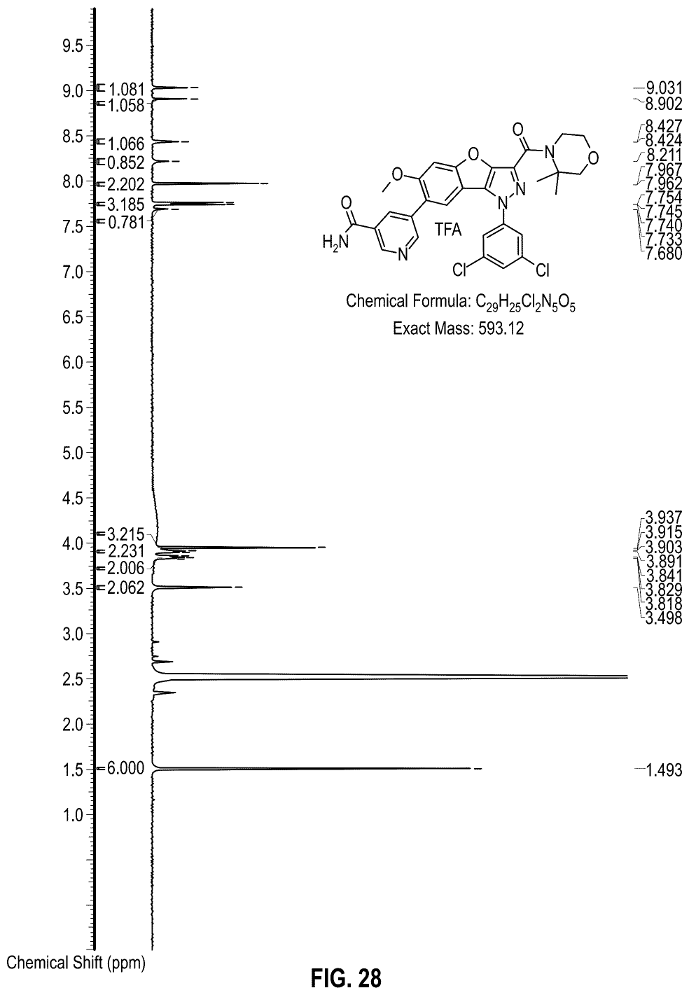

FIG. 29 shows the nuclear magnetic resonance of Compound 4-02A.

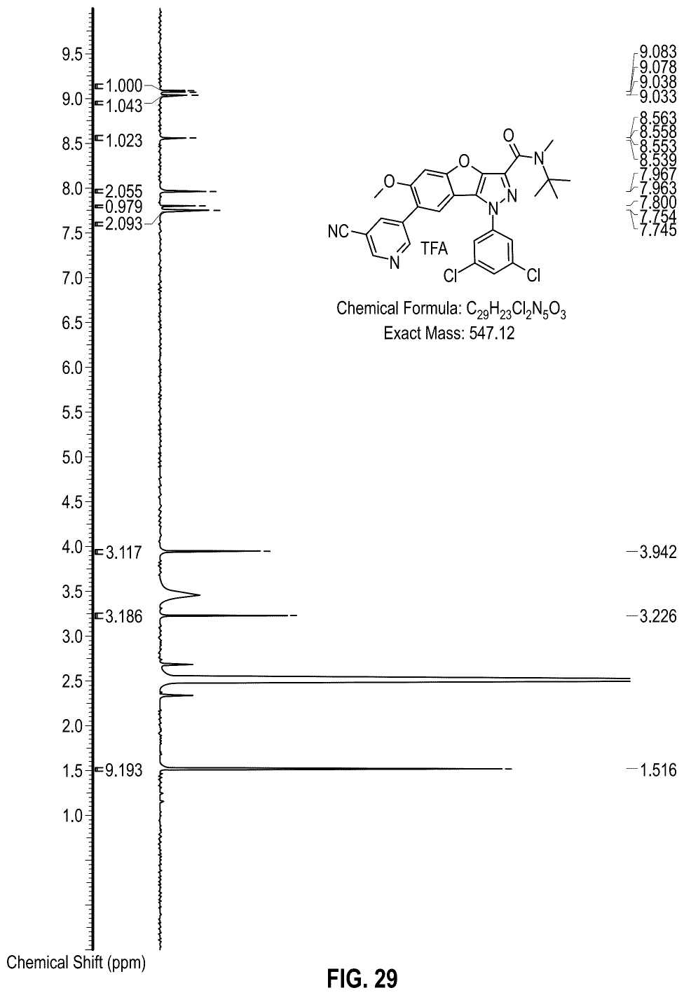

FIG. 30 shows the nuclear magnetic resonance of Compound 4-02.

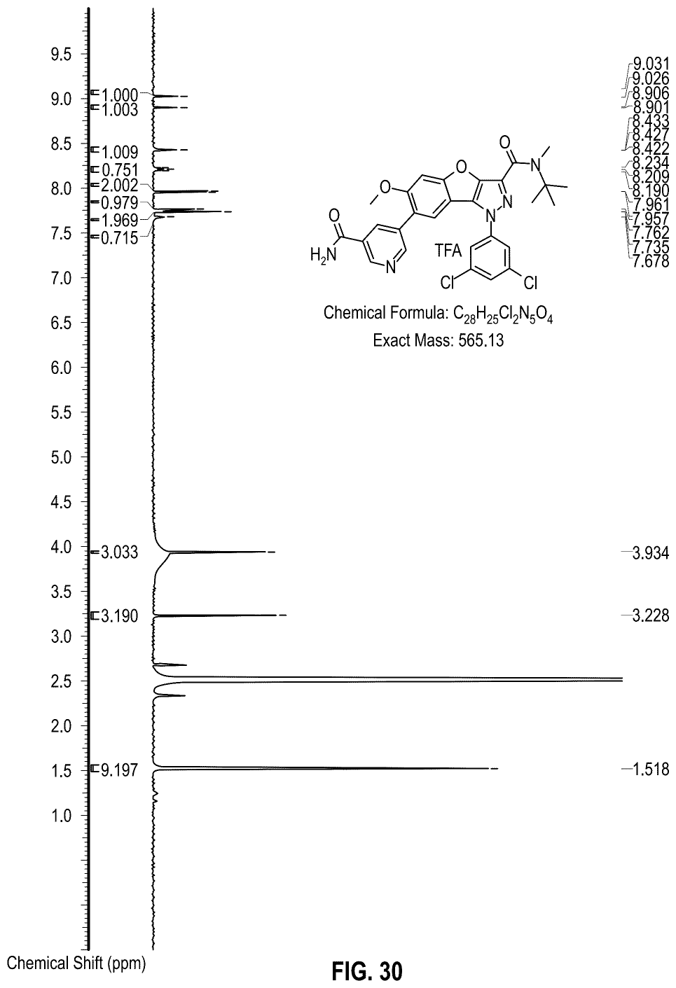

FIG. 31 shows the nuclear magnetic resonance of Compound 4-03A.

FIG. 32 shows the nuclear magnetic resonance of Compound 4-03.

FIG. 33 shows the nuclear magnetic resonance of Compound 4-04A.

FIG. 34 shows the nuclear magnetic resonance of Compound 4-04.

FIG. 35 shows the nuclear magnetic resonance of Compound 4-05A.

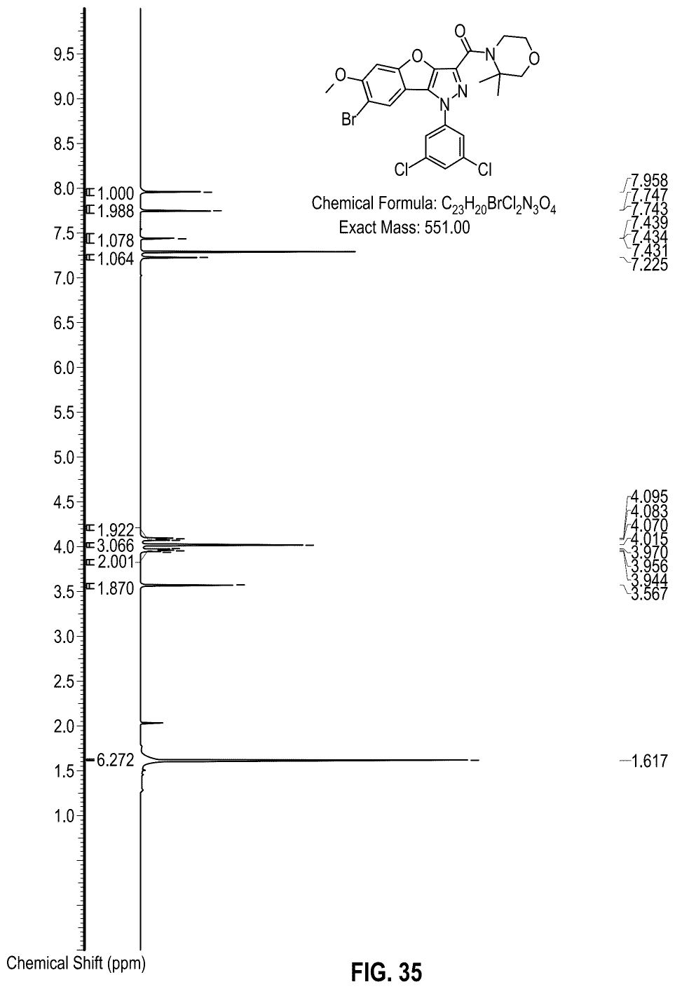

FIG. 36 shows the nuclear magnetic resonance of Compound 4-05.

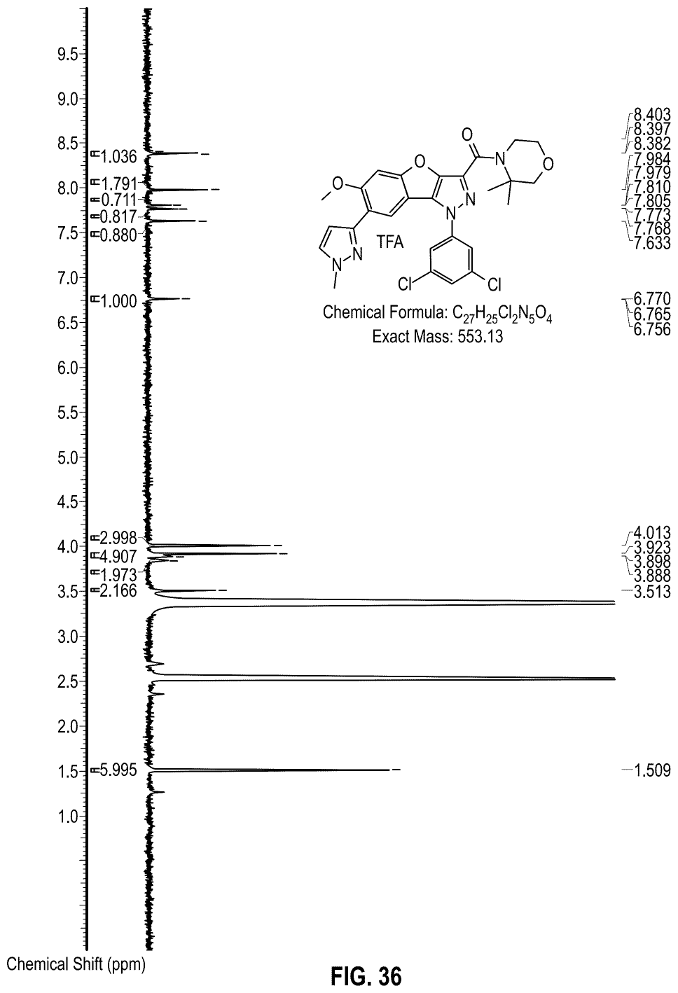

FIG. 37 shows the nuclear magnetic resonance of Compound 4-06A.

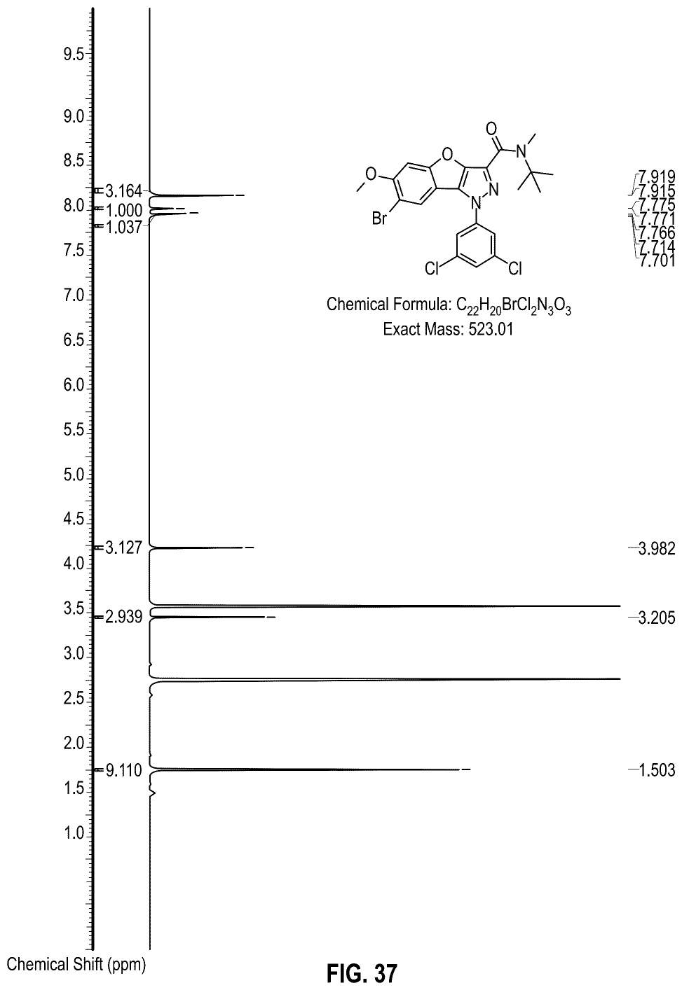

FIG. 38 shows the nuclear magnetic resonance of Compound 4-06.

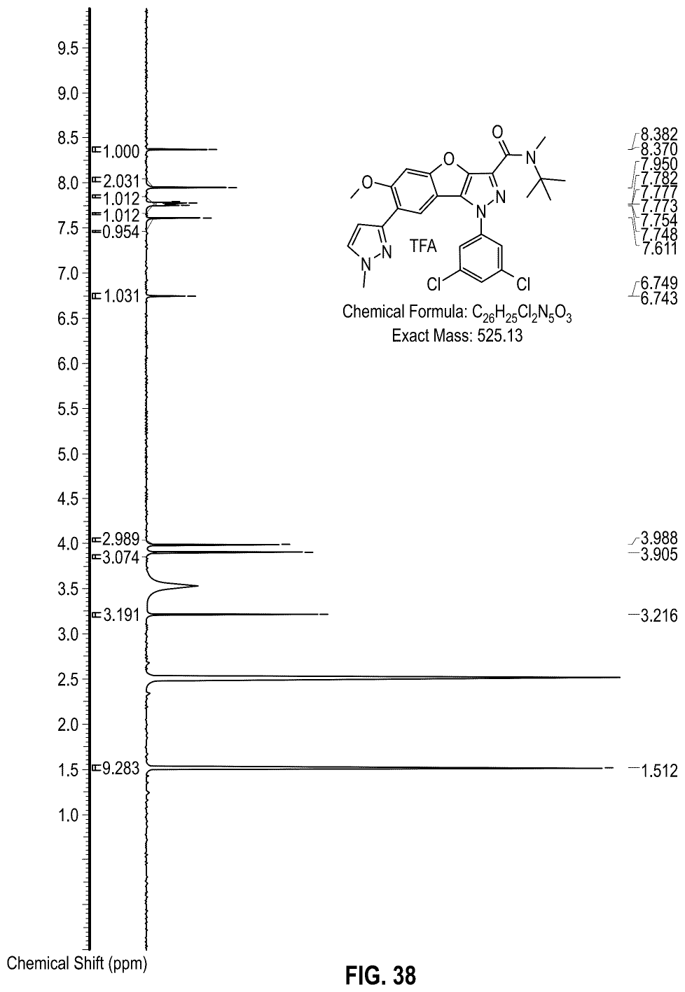

FIG. 39 shows the nuclear magnetic resonance of Compound 4-07A.

FIG. 40 shows the nuclear magnetic resonance of Compound 4-07.

FIG. 41 shows the nuclear magnetic resonance of Compound 4-08A.

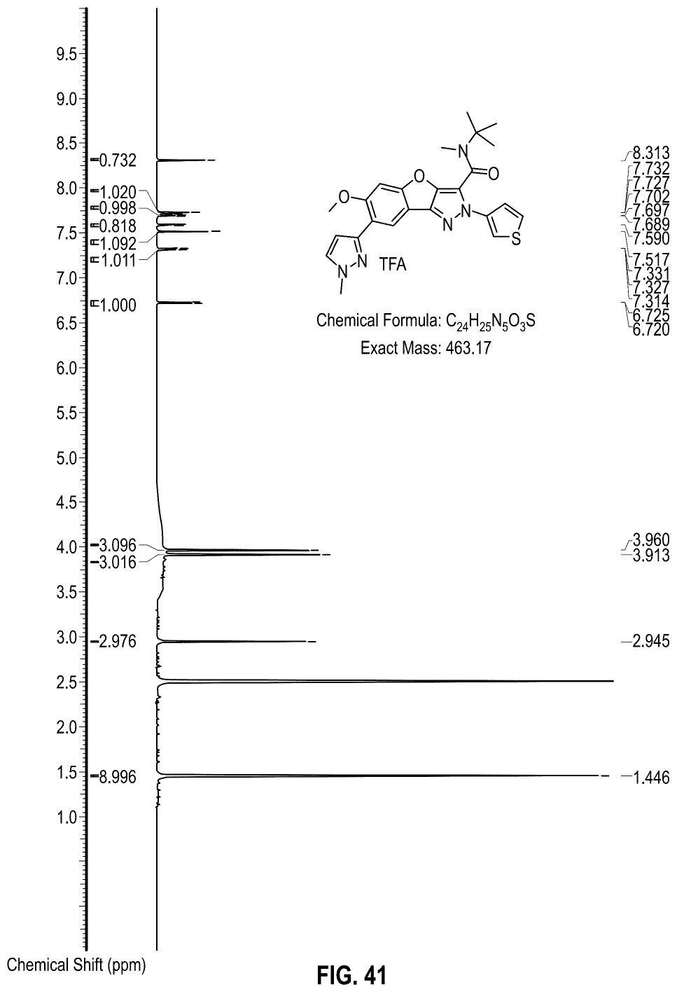

FIG. 42 shows the nuclear magnetic resonance of Compound 4-08.

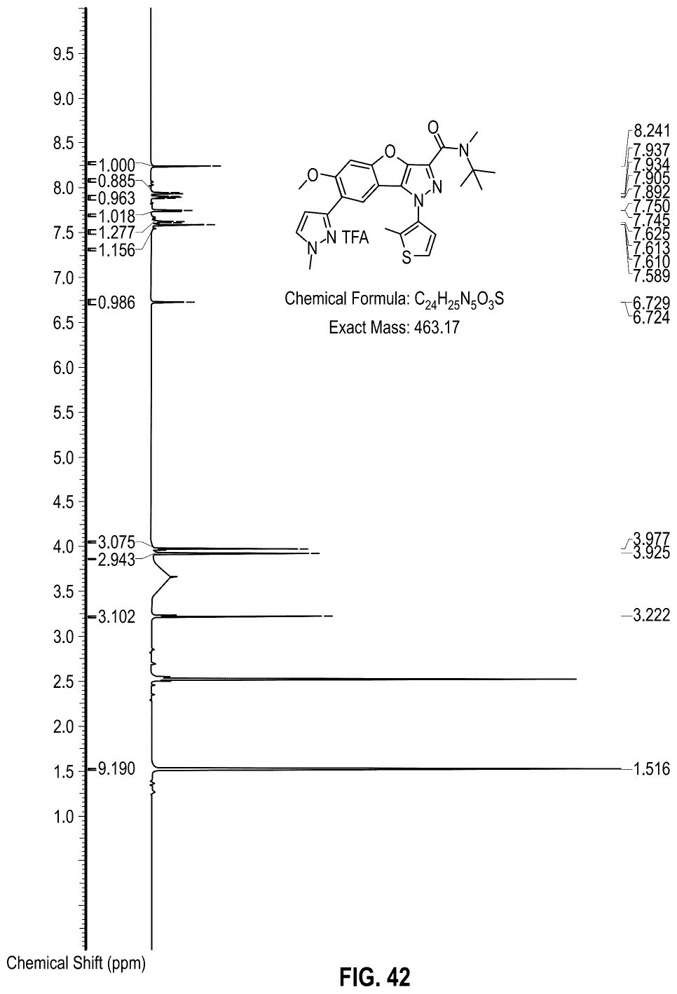

FIG. 43 shows the nuclear magnetic resonance of Compound 5-01.

FIG. 44 shows the nuclear magnetic resonance of Compound 5-02.

FIG. 45 shows the nuclear magnetic resonance of Compound 5-03.

FIG. 46 shows the nuclear magnetic resonance of Compound 5-04.

FIG. 47 shows the nuclear magnetic resonance of Compound 5-05.

FIG. 48 shows the nuclear magnetic resonance of Compound 5-06.

FIG. 49 shows the nuclear magnetic resonance of Compound 5-07.

FIG. 50 shows the nuclear magnetic resonance of Compound 5-08.

FIG. 51 shows the nuclear magnetic resonance of Compound 6-01A.

FIG. 52 shows the nuclear magnetic resonance of Compound 6-01B.

FIG. 53 shows the nuclear magnetic resonance of Compound 6-01.

FIG. 54 shows the nuclear magnetic resonance of Compound 6-02A.

FIG. 55 shows the nuclear magnetic resonance of Compound 6-02B.

FIG. 56 shows the nuclear magnetic resonance of Compound 6-02.

FIG. 57 shows the nuclear magnetic resonance of Compound 6-03.

FIG. 58 shows the nuclear magnetic resonance of Compound 6-04.

FIG. 59 shows the nuclear magnetic resonance of Compound 6-05.

FIG. 60 shows the nuclear magnetic resonance of Compound 6-06.

FIG. 61 shows the nuclear magnetic resonance of Compound 6-07.

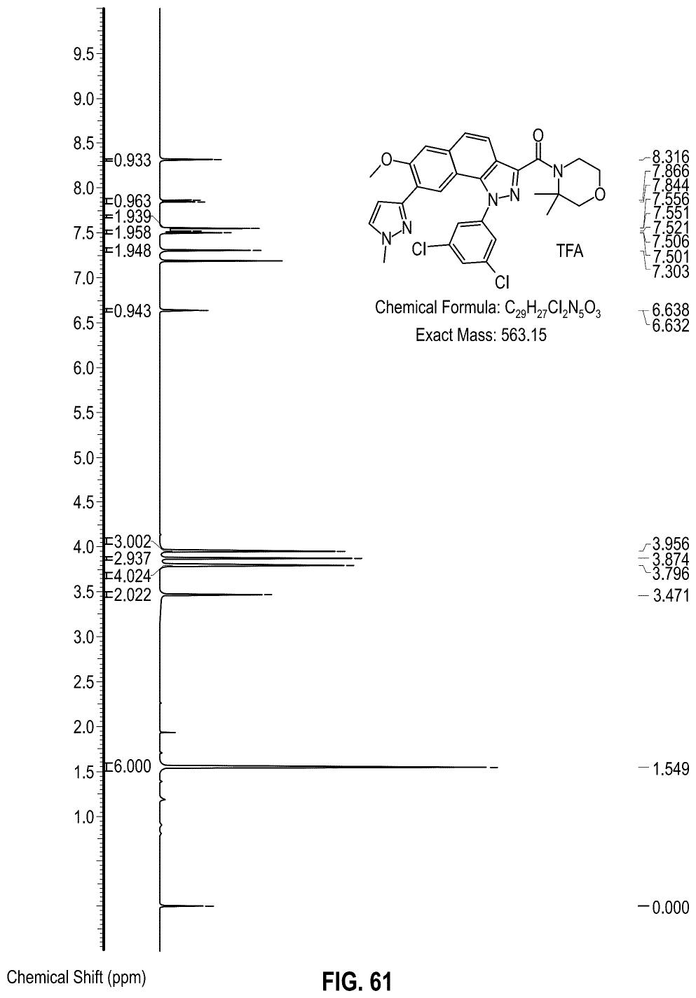

FIG. 62 shows the nuclear magnetic resonance of Compound 6-08.

FIG. 63 shows the nuclear magnetic resonance of Compound 8-01.

FIG. 64 shows the nuclear magnetic resonance of Compound 8-02.

FIG. 65 shows the nuclear magnetic resonance of Compound 8-03.

FIG. 66 shows the nuclear magnetic resonance of Compound 8-05.

FIG. 67 shows the nuclear magnetic resonance of Compound 8-06.

FIG. 68 shows the nuclear magnetic resonance of Compound 8-07A.

FIG. 69 shows the nuclear magnetic resonance of Compound 8-07.

FIG. 70 shows the nuclear magnetic resonance of Compound 8-09.

FIG. 71 shows the nuclear magnetic resonance of Compound 8-10.

FIG. 72 shows the nuclear magnetic resonance of Compound 8-14.

FIG. 73 shows the nuclear magnetic resonance of Compound 8-15.

FIG. 74 shows the nuclear magnetic resonance of Compound 8-16B.

FIG. 75 shows the nuclear magnetic resonance of Compound 8-16.

FIG. 76 shows the nuclear magnetic resonance of Compound 8-17.

FIG. 77 shows the nuclear magnetic resonance of Compound 8-20.

FIG. 78 shows the nuclear magnetic resonance of Compound 8-21.

FIG. 79 shows the nuclear magnetic resonance of Compound 8-22.

FIG. 80 shows the nuclear magnetic resonance of Compound 8-23.

FIG. 81 shows the nuclear magnetic resonance of Compound 8-24.

FIG. 82 shows the nuclear magnetic resonance of Compound 8-25.

FIG. 83 shows the nuclear magnetic resonance of Compound 8-26A.

FIG. 84 shows the nuclear magnetic resonance of Compound 8-26.

FIG. 85 shows the nuclear magnetic resonance of Compound 8-27.

FIG. 86 shows the nuclear magnetic resonance of Compound 8-28.

FIG. 87 shows the nuclear magnetic resonance of Compound 8-29.

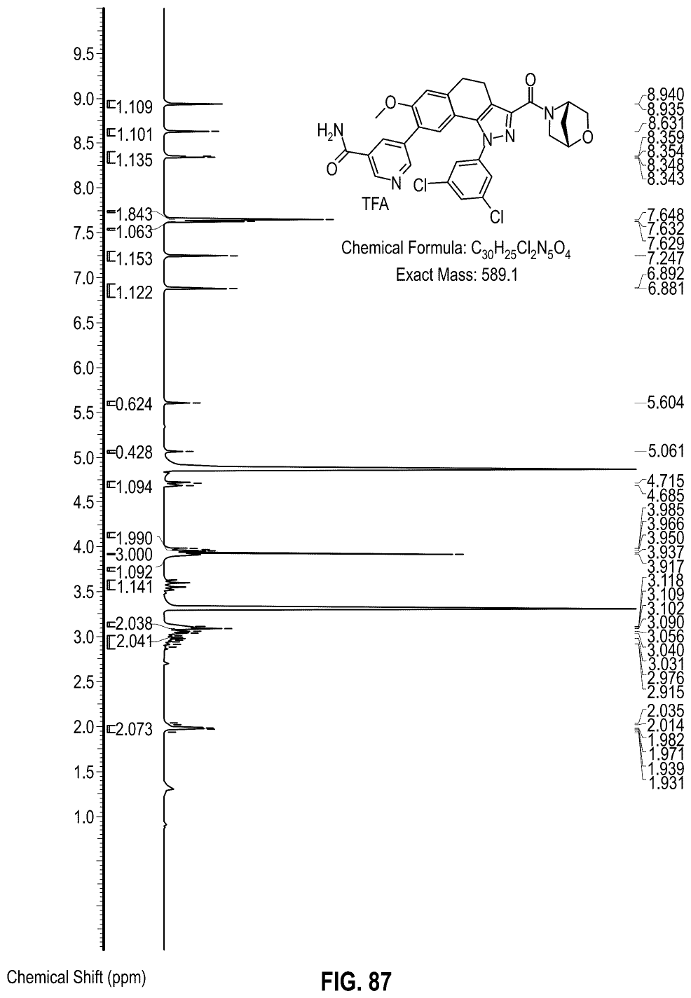

FIG. 88 shows the nuclear magnetic resonance of Compound 8-30.

FIG. 89 shows the nuclear magnetic resonance of Compound 8-31.

FIG. 90 shows the nuclear magnetic resonance of Compound 8-32.

FIG. 91 shows the nuclear magnetic resonance of Compound 8-33.

FIG. 92 shows the nuclear magnetic resonance of Compound 8-34.

FIG. 93 shows the nuclear magnetic resonance of Compound 8-39.

FIG. 94 shows the nuclear magnetic resonance of Compound 8-44.

FIG. 95 shows the nuclear magnetic resonance of Compound 8-77.

FIG. 96 shows the nuclear magnetic resonance of Compound 8-75.

FIG. 97 shows the nuclear magnetic resonance of Compound 8-76.

FIG. 98 shows the nuclear magnetic resonance of Compound 8-78.

FIG. 99 shows the nuclear magnetic resonance of Compound 8-81.

FIG. 100 shows the nuclear magnetic resonance of Compound 8-61.

FIG. 101 shows the nuclear magnetic resonance of Compound 8-60.

FIG. 102 shows the nuclear magnetic resonance of Compound 8-63.

FIG. 103 shows the nuclear magnetic resonance of Compound 8-58.

FIG. 104 shows the nuclear magnetic resonance of Compound 8-51.

FIG. 105 shows the nuclear magnetic resonance of Compound 8-67.

FIG. 106 shows the nuclear magnetic resonance of Compound 8-74.

FIG. 107 shows the nuclear magnetic resonance of Compound 8-4.

FIG. 108 shows the nuclear magnetic resonance of Compound 8-8.

FIG. 109 shows the nuclear magnetic resonance of Compound 8-4a.

FIG. 110 shows the nuclear magnetic resonance of Compound 8-13.

FIG. 111 shows the nuclear magnetic resonance of Compound 8-57.

FIG. 112 shows the nuclear magnetic resonance of Compound 8-18.

FIG. 113 shows the nuclear magnetic resonance of Compound 8-35.

FIG. 114 shows the nuclear magnetic resonance of Compound 8-36.

FIG. 115 shows the nuclear magnetic resonance of Compound 8-37.

FIG. 116 shows the nuclear magnetic resonance of Compound 8-38.

FIG. 117 shows the nuclear magnetic resonance of Compound 8-41.

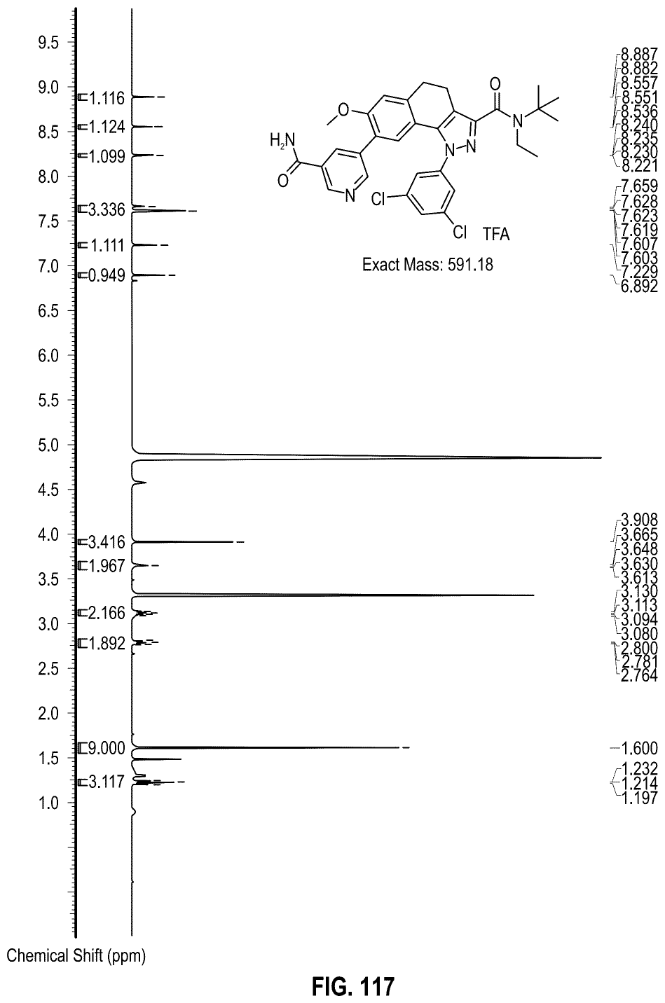

FIG. 118 shows the nuclear magnetic resonance of Compound 8-42.

FIG. 119 shows the nuclear magnetic resonance of Compound 8-43.

FIG. 120 shows the nuclear magnetic resonance of Compound 8-45.

FIG. 121 shows the nuclear magnetic resonance of Compound 8-46.

FIG. 122 shows the nuclear magnetic resonance of Compound 8-47.

FIG. 123 shows the nuclear magnetic resonance of Compound 8-49.

FIG. 124 shows the nuclear magnetic resonance of Compound 8-50.

FIG. 125 shows the nuclear magnetic resonance of Compound 8-52A.

FIG. 126 shows the nuclear magnetic resonance of Compound 8-54A.

FIG. 127 shows the nuclear magnetic resonance of Compound 8-55.

FIG. 128 shows the nuclear magnetic resonance of Compound 8-56.

FIG. 129 shows the nuclear magnetic resonance of Compound 8-62.

FIG. 130 shows the nuclear magnetic resonance of Compound 8-64.

FIG. 131 shows the nuclear magnetic resonance of Compound 8-65.

FIG. 132 shows the nuclear magnetic resonance of Compound 8-69.

FIG. 133 shows the nuclear magnetic resonance of Compound 8-70.

FIG. 134 shows the nuclear magnetic resonance of Compound 8-71.

FIG. 135 shows the nuclear magnetic resonance of Compound 8-79.

FIG. 136 shows the nuclear magnetic resonance of Compound 8-82.

FIG. 137 shows the nuclear magnetic resonance of Compound 8-83.

FIG. 138 shows the nuclear magnetic resonance of Compound 8-84.

FIG. 139 shows the nuclear magnetic resonance of Compound 8-86.

FIG. 140 shows the nuclear magnetic resonance of Compound 8-87.

FIG. 141 shows the nuclear magnetic resonance of Compound 8-89.

FIG. 142 shows the nuclear magnetic resonance of Compound 9-13.

FIG. 143 shows the nuclear magnetic resonance of Compound 9-21.

FIG. 144 shows the nuclear magnetic resonance of Compound 9-4.

FIG. 145 shows the nuclear magnetic resonance of Compound 9-5.

FIG. 146 shows the nuclear magnetic resonance of Compound 9-11.

FIG. 147 shows the nuclear magnetic resonance of Compound 9-14.

FIG. 148 shows the nuclear magnetic resonance of Compound 9-9.

FIG. 149 shows the nuclear magnetic resonance of Compound 9-15.

FIG. 150 shows the nuclear magnetic resonance of Compound 9-2.

FIG. 151 shows the nuclear magnetic resonance of Compound 9-7.

FIG. 152 shows the nuclear magnetic resonance of Compound 9-12.

FIG. 153 shows the nuclear magnetic resonance of Compound 9-16.

FIG. 154 shows the nuclear magnetic resonance of Compound 9-17.

FIG. 155 shows the nuclear magnetic resonance of Compound 9-18.

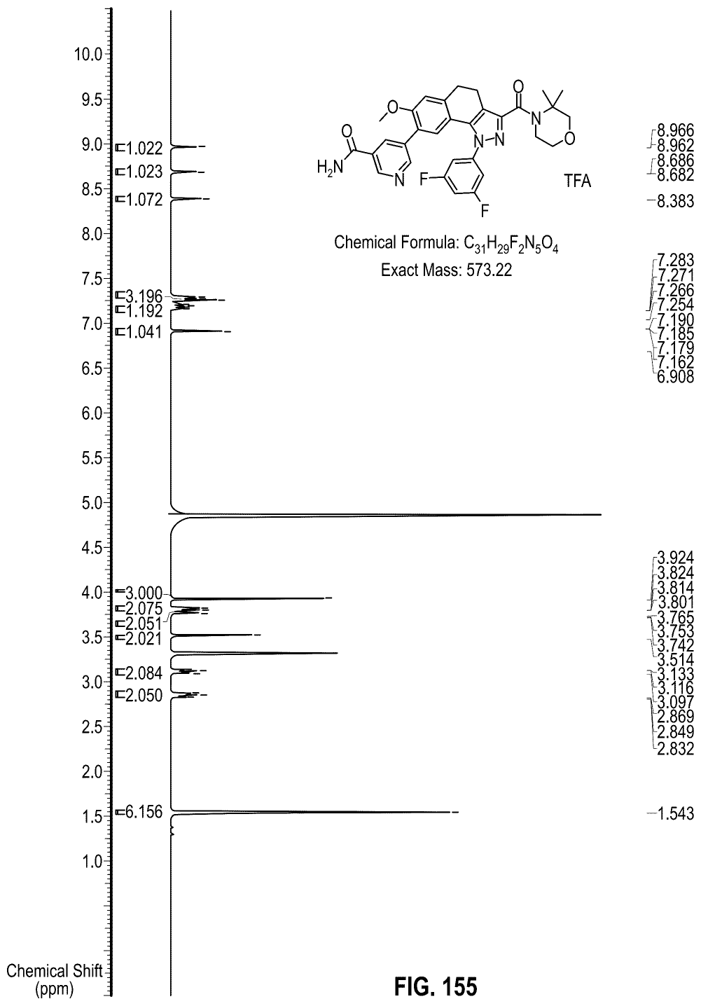

FIG. 156 shows the nuclear magnetic resonance of Compound 9-19.

FIG. 157 shows the nuclear magnetic resonance of Compound 9-20.

FIG. 158 shows the nuclear magnetic resonance of Compound 10-1.

FIG. 159 shows the nuclear magnetic resonance of Compound 10-2.

FIG. 160 shows the nuclear magnetic resonance of Compound 10-3.

FIG. 161 shows the nuclear magnetic resonance of Compound 10-6.

FIG. 162 shows the nuclear magnetic resonance of Compound 10-7.

FIG. 163 shows the nuclear magnetic resonance of Compound 10-8.

FIG. 164 shows the nuclear magnetic resonance of Compound 10-9.

FIG. 165 shows the nuclear magnetic resonance of Compound 10-10.

FIG. 166 shows the nuclear magnetic resonance of Compound 11-1A.

FIG. 167 shows the nuclear magnetic resonance of Compound 11-2.

FIG. 168 shows the nuclear magnetic resonance of Compound 11-1.

FIG. 169 shows the nuclear magnetic resonance of Compound 11-3.

FIG. 170 shows the nuclear magnetic resonance of Compound 12-2.

FIG. 171 shows the nuclear magnetic resonance of Compound 12-23.

FIG. 172 shows the nuclear magnetic resonance of Compound 12-13.

FIG. 173 shows the nuclear magnetic resonance of Compound 12-15.

FIG. 174 shows the nuclear magnetic resonance of Compound 12-16.

FIG. 175 shows the nuclear magnetic resonance of Compound 12-1.

FIG. 176 shows the nuclear magnetic resonance of Compound 12-4.

FIG. 177 shows the nuclear magnetic resonance of Compound 12-18.

FIG. 178 shows the nuclear magnetic resonance of Compound 12-19.

FIG. 179 shows the nuclear magnetic resonance of Compound 13-1.

FIG. 180 shows the nuclear magnetic resonance of Compound 13-4.

FIG. 181 shows the nuclear magnetic resonance of Compound 13-9.

FIG. 182 shows the nuclear magnetic resonance of Compound 13-7.

FIG. 183 shows the nuclear magnetic resonance of Compound 13-8.

FIG. 184 shows the nuclear magnetic resonance of Compound 13-2.

FIG. 185 shows the nuclear magnetic resonance of Compound 13-5.

FIG. 186 shows the nuclear magnetic resonance of Compound 15-1.

FIG. 187 shows the nuclear magnetic resonance of Compound 15-3.

FIG. 188 shows the nuclear magnetic resonance of Compound 15-4.

FIG. 189 shows the nuclear magnetic resonance of Compound 15-5.

FIG. 190 shows the nuclear magnetic resonance of Compound 15-9.

FIG. 191 shows the nuclear magnetic resonance of Compound 15-2.

FIG. 192 shows the nuclear magnetic resonance of Compound 15-6.

FIG. 193 shows the nuclear magnetic resonance of Compound 15-10.

### DETAILED DESCRIPTION

### Definitions

The terminology used herein is for the purpose of describing particular cases only and is not intended to be limiting. In this application, the use of the singular includes the plural unless specifically stated otherwise.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed disclosure.

The abbreviations used herein have their conventional meaning within the chemical and biological arts, unless otherwise specified. The chemical structures and formulae set forth herein are constructed according to the standard rules of chemical valency known in the chemical arts.

The symbol “ [symbol] ” denotes the point of attachment of a chemical moiety to the remainder of a molecule or chemical formula.

As used herein, the terms “about” or “approximately” can mean within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein, the term “derivative” indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto. Non-limiting examples of “derivatives” can include a prodrug, a metabolite, an enantiomer, a diastereomer, esters (e.g. acyloxyalkyl esters, alkoxycarbonyloxyalkyl esters, alkyl esters, aryl esters, phosphate esters, sulfonate esters, sulfate esters and disulfide containing esters), ethers, amides, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts, sulfonate esters, and the like. In some cases, a derivative may include trivial substitutions (i.e. additional alkyl/akylene groups) to a parent compound that retains the chemical and/or biological activity of the parent compound.

As used herein, the term “pharmaceutically acceptable salt” generally refers to an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an example range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

As used herein, the term “aromatic” generally refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

As used herein, the term “Halo” or “halogen” generally refers to bromo, chloro, fluoro or iodo.

As used herein, the term “Haloalkyl” generally refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

As used herein, the term “Haloalkoxy” generally refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

As used herein, the term “fluoroalkyl” generally refers to an alkyl group in which one or more hydrogen atoms are replaced by fluorine.

As used herein, the term “tautomer” generally refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers may exist. All tautomeric forms of the compounds disclosed herein are contemplated. The exact ratio of the tautomers depends on several factors, including temperature, solvent, and pH. Some examples of tautomeric interconversions include:

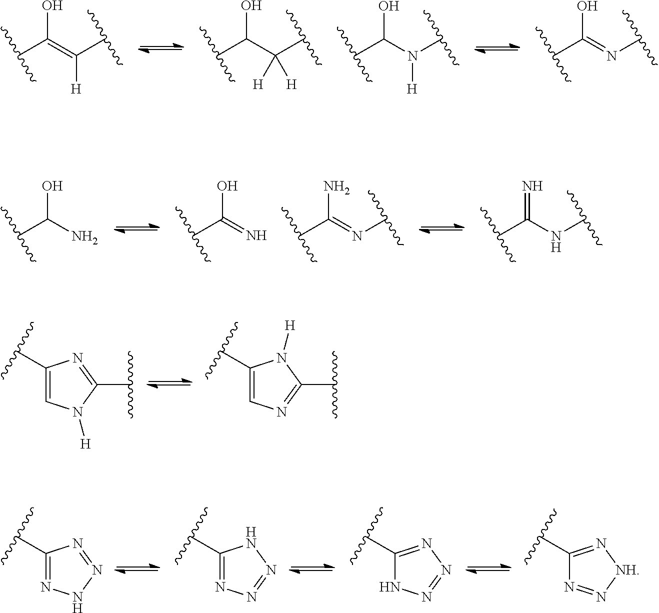

As used herein, the terms “effective amount” or “therapeutically effective amount,” generally refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts may depend on the purpose of the treatment, and may be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Example substituents include amino, alkylamino, and the like.

As used herein, the term “substituent” generally refers to positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “=O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “alkyl” generally refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

As used herein, the term “aryl” refers to a radical derived from a hydrocarbon ring system comprising at least one aromatic ring. In some embodiments, an aryl comprises hydrogens and 6 to 30 carbon atoms. The aryl radical can be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

As used herein, the term “alkenyl” generally refers to a type of alkyl group in which at least one carbon-carbon double bond is present. In one embodiment, an alkenyl group has the formula —C(R^{a})=CR^{a}₂, wherein R^{a} refers to the remaining portions of the alkenyl group, which may be the same or different. In some embodiments, R^{a} is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂. “Alkenylene” or “alkenylene chain” refers to a alkylene group in which at least one carbon-carbon double bond is present. In some embodiments, the alkenylene is —CH═CH—, —CH₂CH₂CH═CH—, or —CH═CHCH₂CH₂—. In some embodiments, the alkenylene is —CH═CH—. In some embodiments, the alkenylene is —CH₂CH₂CH═CH—. In some embodiments, the alkenylene is —CH═CHCH₂CH₂—.

As used herein, the term “alkynyl” generally refers to a type of alkyl group in which at least one carbon-carbon triple bond is present. In one embodiment, an alkynyl group has the formula —C≡CR^{a}, wherein R^{a} refers to the remaining portions of the alkynyl group. In some embodiments, R^{a} is H or an alkyl. In some embodiments, an alkynyl is selected from ethynyl (i.e., acetylenyl), propynyl (i.e., propargyl), butynyl, pentynyl, and the like. Non-limiting examples of an alkynyl group include —C≡CH, —C≡CCH₃, and —CH₂C≡CH. “Alkynylene” or “alkynylene chain” refers to a alkylene group in which at least one carbon-carbon triple bond is present. In some embodiments, the alkynylene is —C≡C—, —CH₂CH₂C≡C—, or —C≡CCH₂CH₂—. In some embodiments, the alkynylene is —C≡C—. In some embodiments, the alkynylene is —CH₂CH₂C≡C—. In some embodiments, the alkynylene is —C≡CCH₂CH₂—.

As used herein, the term “cycloalkyl” generally refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

As used herein, the term “heterocycle” or “heterocyclic” generally refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, ₃ h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

As used herein, the term “heterocycloalkyl” generally refers to a cycloalkyl group that includes at least one ring heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-2 S atoms in the ring. In some embodiments, heterocycloalkyls have from 2 to 12 carbons, 1-3 N atoms, 0-2 O atoms, and 0-2 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

As used herein, the term “heteroaryl” generally refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉heteroaryl. In some embodiments, a heteroaryl group is partially reduced to form a heterocycloalkyl group defined herein. In some embodiments, a heteroaryl group is fully reduced to form a heterocycloalkyl group defined herein.

As used herein, the term “heteroalkyl” generally refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted. Representative heteroalkylene groups include, but are not limited to —OCH₂CH₂O—, —OCH₂CH₂OCH₂CH₂O—, or —OCH₂CH₂OCH₂CH₂OCH₂CH₂O—.

As used herein, the term “heteroalkenyl” refers to an alkenyl group in which one or more skeletal atoms of the alkenyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkenyl is attached to the rest of the molecule at a carbon atom of the heteroalkenyl. In some embodiments, a heteroalkenyl is attached to the rest of the molecule at a heteroatom of the heteroalkenyl. In some embodiments, a heteroalkyl is a C₁-C₆ heteroalkenyl.

As used herein, the term “heteroalkynyl” refers to an alkynyl group in which one or more skeletal atoms of the alkynyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkynyl is attached to the rest of the molecule at a carbon atom of the heteroalkynyl. In some embodiments, a heteroalkynyl is attached to the rest of the molecule at a heteroatom of the heteroalkynyl. In some embodiments, a heteroalkyl is a C₁-C₆ heteroalkynyl.

As used herein, the term “heteroatom” or “ring heteroatom” generally refers to an atom including oxygen (O), nitrogen (N), sulfur (S), phosphorus (P), and silicon (Si), or any combination thereof.

As used herein, the term “substituent group,” refers to a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

### FSH Modulators

For ovulation induction, the only approved medication to date is clomiphene citrate, a mixture of estrogen agonist and antagonist isomers that are used to increase endogenous FSH and LH release. The objective of clomiphene prescription is to increase endogenous plasma FSH levels and LH levels to a level sufficient to support the development of one or two follicles to the point of ovulation. The mechanism of clomiphene or off-label use of aromatase inhibitors is indirect; it assumes a homogeneous response of the hypothalamic-pituitary-ovarian axis across patient subgroups to achieve comparable responders across patients. FSH modulators (e.g. orally active FSH receptor agonists) described herein can show improved ovulation induction with low doses of FSH agonist, for example by directly acting on the ovary and without requiring hypothalamic-pituitary input for efficacy. In this regard, FSH modulators described herein (e.g.oral FSH receptor agonists) can deliver improved efficacy and precision of control over ovulation induction than can be accomplished through administration of clomiphene (approved) or aromatase inhibitors (off-label).

According to one embodiment, the disclosure relates to a method of allosterically modulating FSH—R activity in a biological sample comprising contacting said biological sample with a compound of this disclosure, or a composition comprising said compound According to another embodiment, the disclosure relates to a method of allosterically modulating FSHR, or a mutant thereof, activity in a biological sample in a positive manner, comprising contacting said biological sample with a compound of this disclosure, or a composition comprising said compound.

The compounds of the disclosure are strong and selective modulators of the FSH receptor. In some instances, their selectivity to the FSH receptor can be 3 to 10-fold over the LH receptor and even 10 to 100-fold over the TSH receptor. In some embodiments, the EC₅₀ or IC₅₀ amounts to more than 10 μM on unrelated G protein-coupled receptors (GPCR) or non-GPCR targets. In some instances, the selectivity of the FSH modulators to the FSH receptor can be 100 to 200-fold over the LH receptor. The current disclosure comprises the use of the compounds of the disclosure in the regulation and/or modulation of the FSHR signal cascade, which can be advantageously applied as research tool, for diagnosis and/or in treatment of any disorder arising fromn FSHR signaling.

For example, the compounds of the disclosure are useful in-vitro as unique tools for understanding the biological role of FSH, including the evaluation of the many factors thought to influence, and be influenced by, the production of FSH and the interaction of FSH with the FSHR (e. g. the mechanism of FSH signal transduction/receptor activation). The present compounds are also useful in the development of other compounds that interact with FSHR since the present compounds provide important structure-activity relationship (SAR) information that facilitate that development. Compounds of the present disclosure that bind to FSHR can be used as reagents for detecting FSHR on living cells, fixed cells, in biological fluids, in tissue homogenates, in purified, natural biological materials, etc. For example, by labeling such compounds, one can identify cells having FSHR on their surfaces. In addition, based on their ability to bind FSHR, compounds of the present disclosure can be used in in-situ staining. FACS (fluorescence-activated cell sorting), western blotting, ELISA (enzyme-linked immunoadsorptive assay), etc. receptor purification or in purifying cells expressing FSHR on the cell surface or inside permeabilized cells. Compounds of the present disclosure that bind to FSHR can also be used to distinguish the effects of FSH that are independent of carbohydrate moieties that comprise the glycoprotein nature of FSH, and to identify essential and alternative cellular responses of the small molecule FSHR agonist relative to the glycoprotein FSH.

The compounds of the disclosure can also be utilized as commercial research reagents for various medical research and diagnostic uses. Such uses can include but are not limited to: use as a calibration standard for quantifying the activities of candidate FSH agonists in a variety of functional assays; use as blocking reagents in random compound screening, i.e. in looking for new families of FSH receptor ligands, the compounds can be used to block recovery of the presently claimed FSH compounds: use in the co-crystallization with FSHR receptor, i.e. the compounds of the present disclosure will allow formation of crystals of the compound bound to FSHR, enabling the determination of receptor/compound structure by x-ray crystallography or cryoEM; other research and diagnostic applications, wherein FSHR is preferably activated or such activation is conveniently calibrated against a known quantity of an FSH agonist, etc.; use in assays as probes for determining the expression of FSHR on the surface of cells; and developing assays for detecting compounds which bind to the same site as the FSHR binding ligands.

The compounds of the disclosure can be applied either themselves and/or in combination with physical measurements for diagnostics of treatment effectiveness. Pharmaceutical compositions containing said compounds and the use of said compounds to treat FSHR-mediated conditions is a promising, novel approach for a broad spectrum of therapies causing a direct and immediate improvement in the state of health, whether in human or animal. The impact is of special benefit to efficiently combat infertility, either alone or in combination with other fertility-inducing treatments.

In particular, the compounds of the disclosure potentiate the native FSH effect for both ovulation induction and assisted reproductive technology. The orally bioavailable and active new chemical entities of the disclosure improve convenience for patients and compliance for physicians.

The compounds of the disclosure are active in the primary screen (CHO with or without FSH), selective in secondary screen (no or low activity against TSHR and LHR) and potent in the granulosa cell estradiol assay. Neither hERG nor any toxic effects can be observed in-vitro.

In certain embodiments, the disclosure provides a method for in-vitro fertilization comprising:
(a) treating a mammal according to the method as described above,
(b) collecting ova from said mammal,
(c) fertilizing said ova, and
(d) implanting said fertilized ova into a host mammal.

Described herein are FSH modulator compounds, pharmaceutically acceptable salts or solvates thereof, pharmaceutical compositions comprising FSH modulator compounds (or pharmaceutically acceptable salts or solvates thereof), lipid nanoparticle compositions comprising FSH modulators, and methods of treating a disease comprising administering FSH modulator compounds, pharmaceutically acceptable salts or solvates thereof, pharmaceutical compositions comprising FSH modulator compounds (or pharmaceutically acceptable salts or solvates thereof), and/or lipid nanoparticle compositions comprising FSH modulators (or pharmaceutically acceptable salts or solvates thereof), as described herein, to a subject in need thereof.

In some embodiments, the compounds of the disclosure can show low activity or no activity on FSHR as agonists. Such low activity agonists can have similar structural features as potent FSHR agonists are antagonists of the glycoprotein hormone FSH. In some embodiments, an inactive (e.g. a low activity) FSHR compound can displace and inhibit the activity of a potent FSHR agonist of a similar structural feature. Such compounds can be useful as a diagnostic too by displacing agonist without having agonist activity itself.

In some instances, inactive compounds of the disclosure can attenuate the activity of a more active compound by displacing the agonist activity of the more active compound. In some embodiments, inactive compounds can be useful, for example, to prevent over-stimulation of agonist response in women and/or to avoid or mitigate ovarian hyperstimulation syndrome.

In some instances, FSH modulators or compositions described herein are compounds of Formula (I):

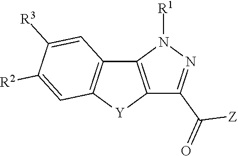

or a pharmaceutically acceptable salts thereof.

In some embodiments, R¹ is C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, Y is —OC(R⁴)₂—.

In some embodiments, Z is —OR⁴, —N(R⁴)₂, —SR⁴, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵.

In some embodiments, R² is —CF₃, —OCF₃, or —OCH₂CH₃.

In some embodiments. R³ is hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, each R⁴ is independently hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, each R⁵ is independently deuterium, halogen, —OH, —NO₂, —CN, —SR⁶, —S(═O)R⁶, —S(═O)₂R⁶, —N(R⁶)₂, —C(═O)R⁶, —OC(═O)R⁶, —C(═O)OR⁶, —C(═O)N(R⁶)₂, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₃-C₇ cycloalkyl, substituted or unsubstituted C₂-C₇heterocycloalkyl, substituted or unsubstituted aryl, and substituted or unsubstituted heteroaryl.

In some embodiments, each R⁶ is independently hydrogen, deuterium, substituted or unsubstituted C₁-C₄ alkyl, -CD₃, substituted or unsubstituted C₁-C₄haloalkyl, substituted or unsubstituted C₁-C₄heteroalkyl, substituted or unsubstituted C₃-C₆ cycloalkyl, substituted or unsubstituted C₂-C₅ heterocycloalkyl, substituted or unsubstituted aryl, or substituted or unsubstituted heteroaryl.

In some embodiments, Y is —O—, —S—, —NR⁴—, —SC(R⁴)₂—, —C(R⁴)₂₀—, —C(R⁴)₂S—, —C(R⁴)₂NR⁴—, —C(R⁴)₂—, —S(═O)C(R⁴)₂—, —C(R⁴)₂S(═O)—, —S(═O)₂C(R⁴)₂—, —C(R⁴)₂S(═O)₂—, or —CR⁴=CR⁴—.

In some embodiments, Z is —OR⁴, —N(R⁴)₂, —SR⁴, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵.

In some embodiments, R² is —R, halogen, -haloalkyl, —OR, —SR, —CN, —NO₂, —CF₃, —OCF₃, —SO₂R, —SOR, —C(O)R, —CO₂R, —C(O)N(R)₂, —NRC(O)R, —NRC(O)N(R)₂, —NRSO₂R, or —N(R)₂;

In some embodiments, Y is —O—, —S—, —NR⁴—, —OC(R⁴)₂—, —SC(R⁴)₂—, —C(R⁴)₂₀—, —C(R⁴)₂S—, C(R⁴)₂NR⁴—, —C(R⁴)₂—, —C(R⁴)₂—C(R⁴)₂— or —CR⁴=CR⁴—.

In some embodiments, R is hydrogen, halogen, —CF₃, —OCF₃, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵.

In some embodiments, Z is —O-t-butyl.

In some embodiments, R³ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, R³ is C₃-C₁₆ heteroaryl unsubstituted or substituted with 1, 2, 3, 4, or 5 groups selected from R⁵.

In some embodiments, R³ is selected from

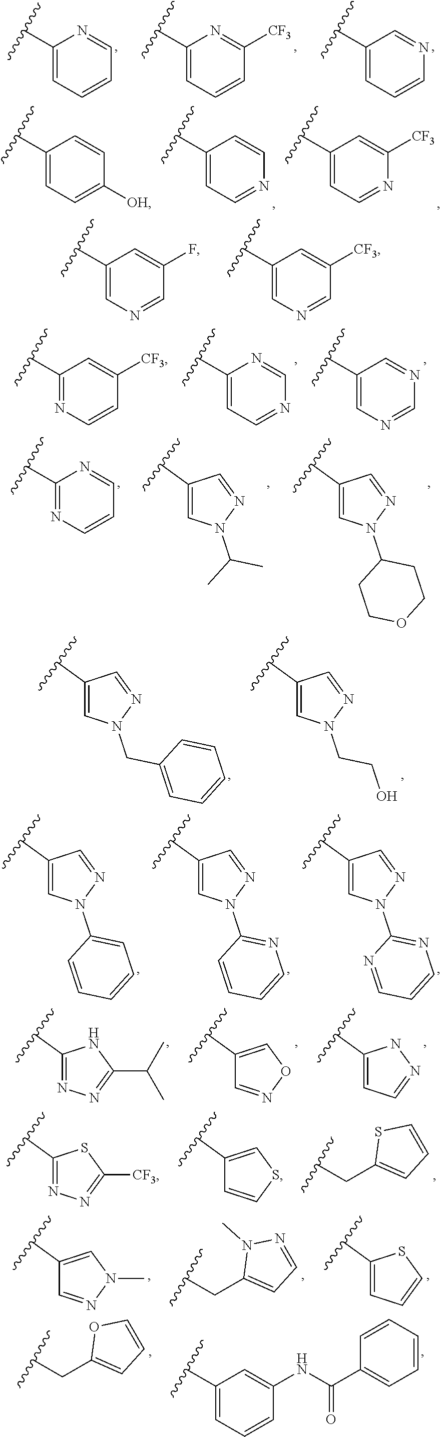

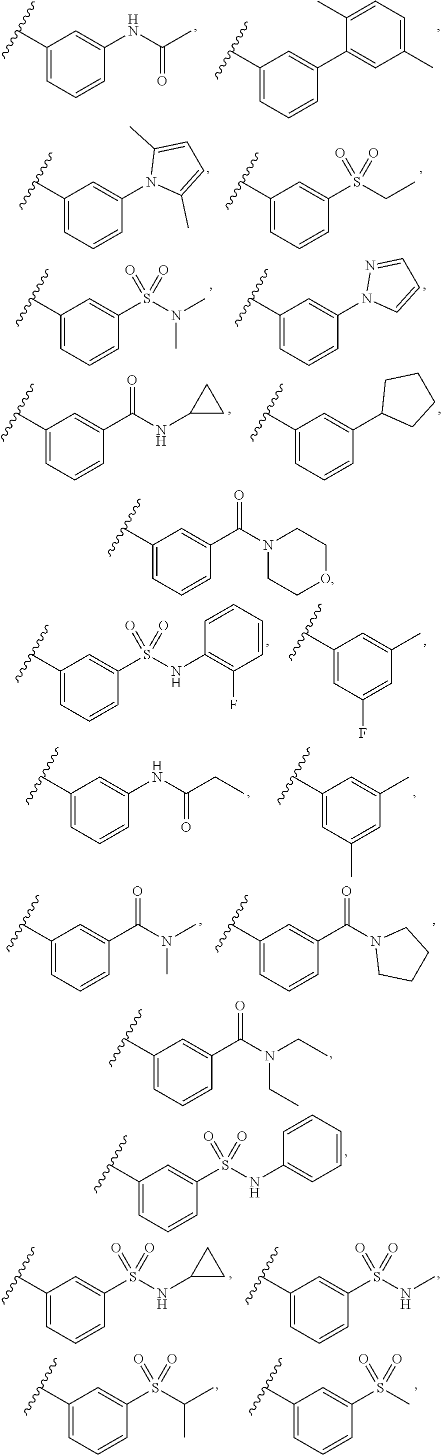

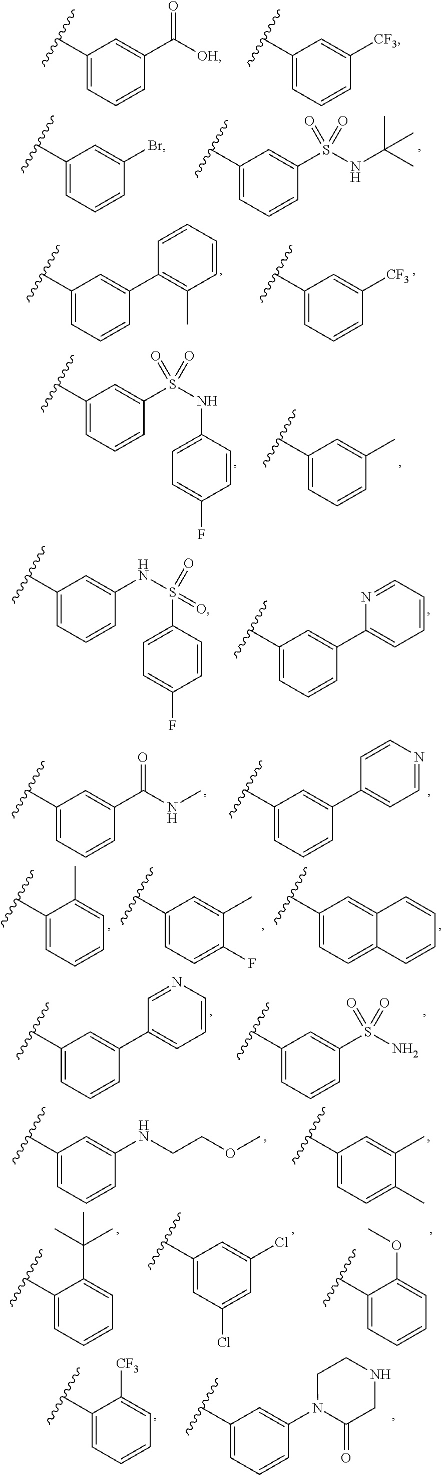

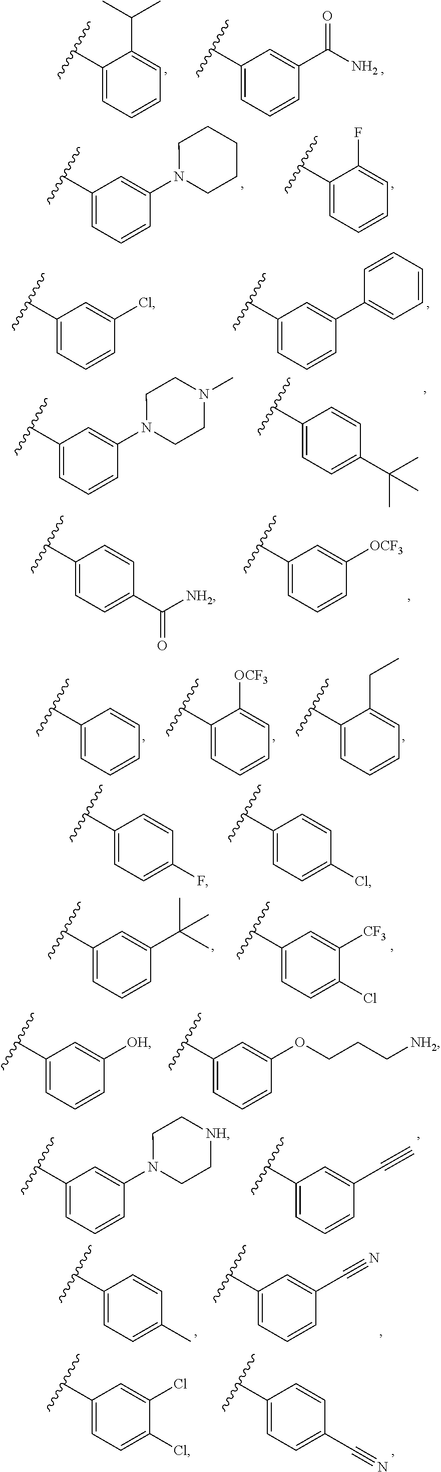

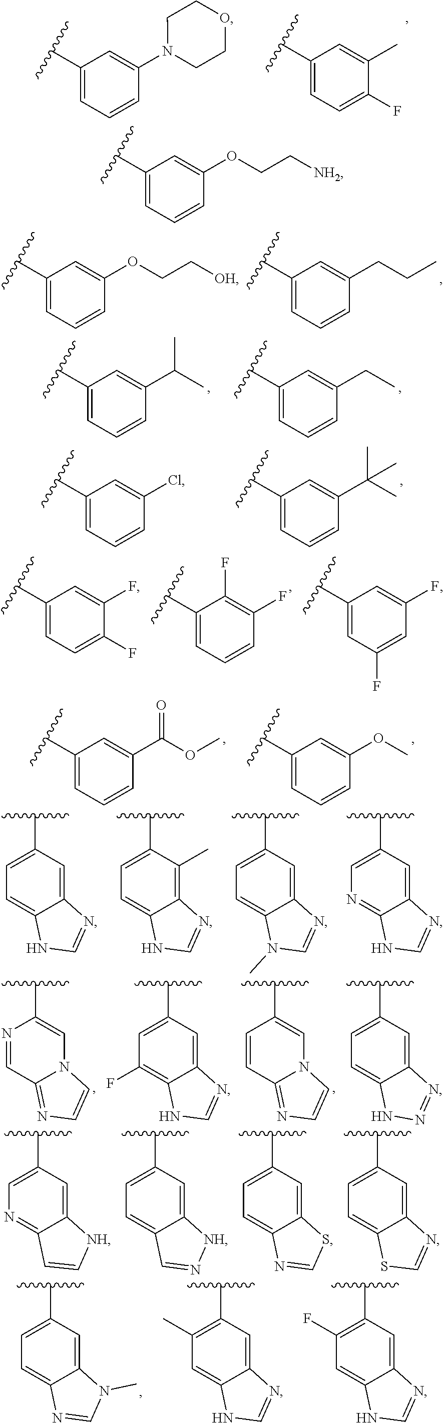

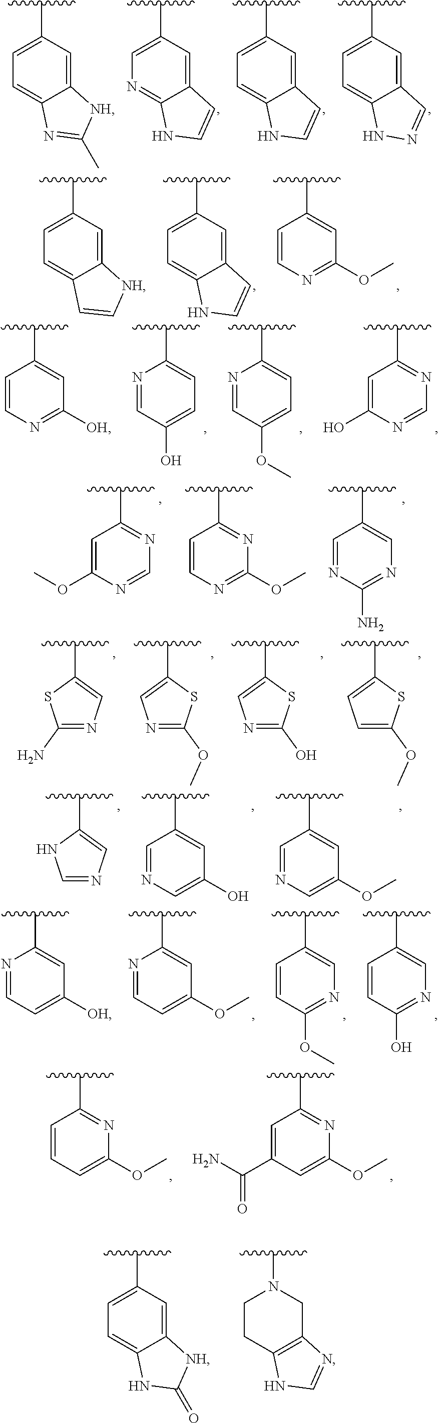

and substitutions thereof.

In some embodiments, R³ is selected from

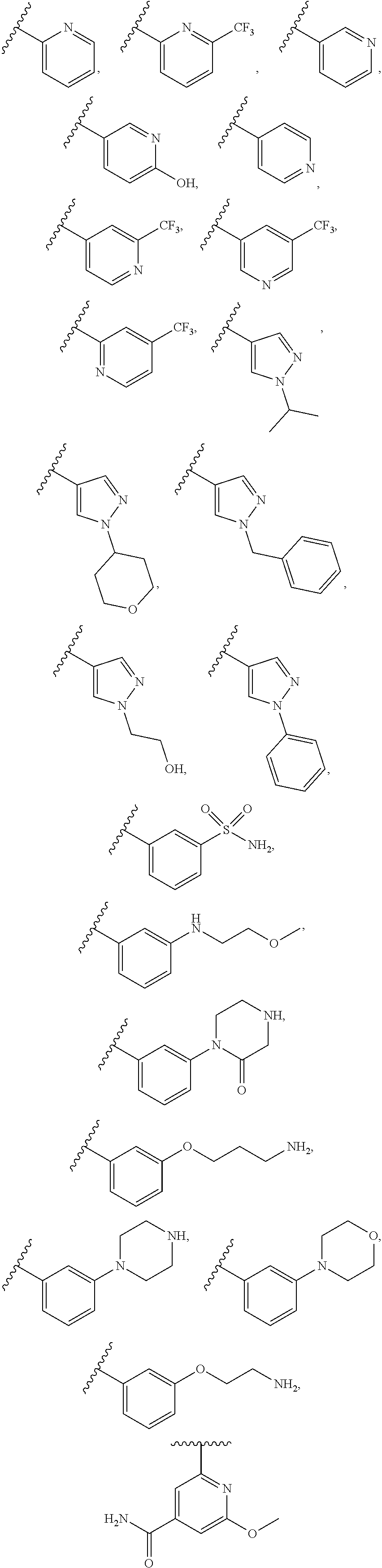

and substitutions thereof.

In some embodiments, R³ is selected from

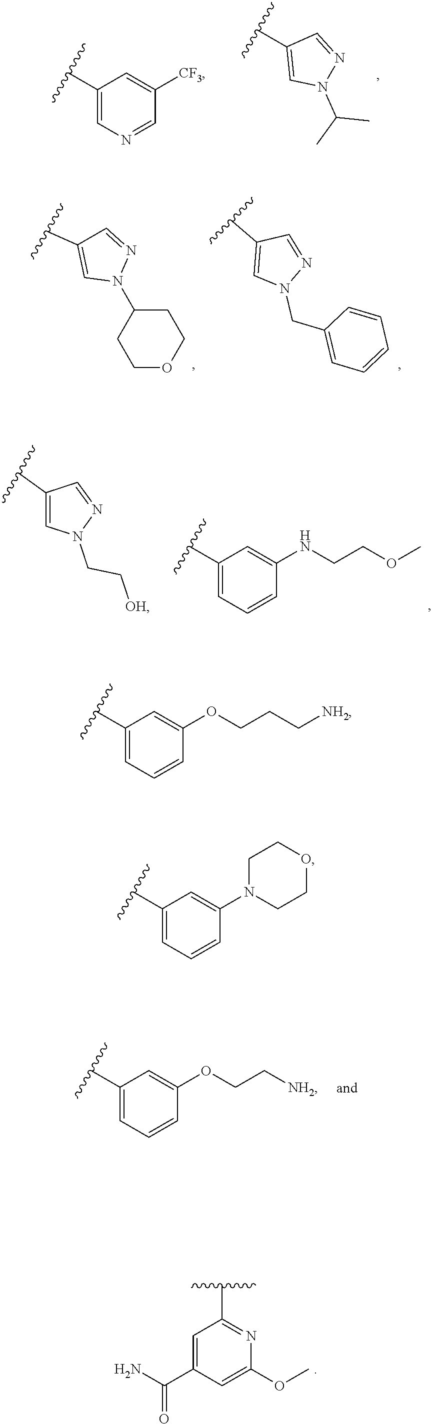

In some embodiments, R³ is

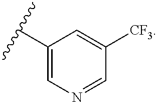 [SMILES: *c1cncc(C(F)(F)F)c1]

In some embodiments, R³ is

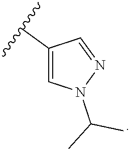 [SMILES: *c1cnn(C(C)C)c1]

In some embodiments, R³ is

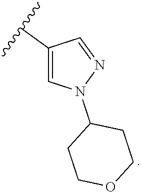 [SMILES: *c1cnn(C2CCOCC2)c1]

In some embodiments, R³ is

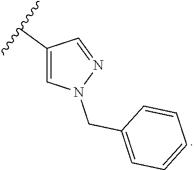 [SMILES: *c1cnn(Cc2ccccc2)c1]

In some embodiments, R³ is

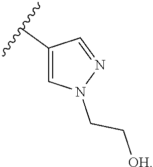 [SMILES: *c1cnn(CCO)c1]

In some embodiments, R³ is

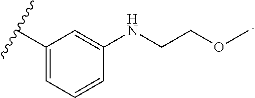 [SMILES: *c1cccc(NCCOC)c1]

In some embodiments, R³ is

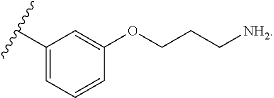 [SMILES: *c1cccc(OCCCN)c1]

In some embodiments, R³ is

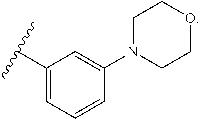 [SMILES: *c1cccc(N2CCOCC2)c1]

In some embodiments, R³ is

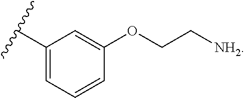 [SMILES: *c1cccc(OCCN)c1]

In some embodiments, R³ is

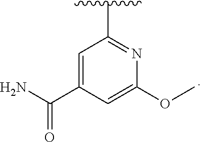 [SMILES: *c1cc(C(N)=O)cc(OC)n1]

In some embodiments, R² is -halogen, —OR, —SR, —CN, —NO₂, —CF₃, —OCF₃, or —C(═O)CH₃.

In some embodiments, R² is —OCH₃, —SCH₃, —CN, —NO₂, —CF₃, or —OCF₃.

In some embodiments, R² is —OCH₃, —SCH₃, or —OCF₃.

In some embodiments, R² is —SCH₃.

In some embodiments, R² is —OCF₃.

In some embodiments, R² is —CF₃.

In some embodiments, R² is —OCH₂CH₃.

In some embodiments, R² is —OCH₃.

In some embodiments, R¹ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, R¹ is C₆ aryl unsubstituted or substituted with 1, 2, 3, 4, or 5 groups selected from R⁵.

In some embodiments, R¹ is C₃-C₁₆ heteroaryl unsubstituted or substituted with 1, 2, 3, 4, or 5 groups selected from R⁵.

In some embodiments R¹ is selected from

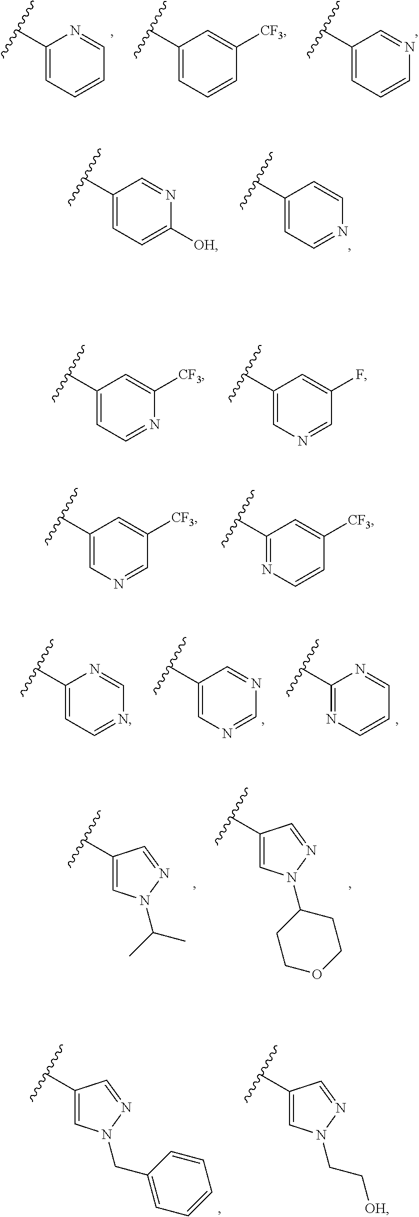

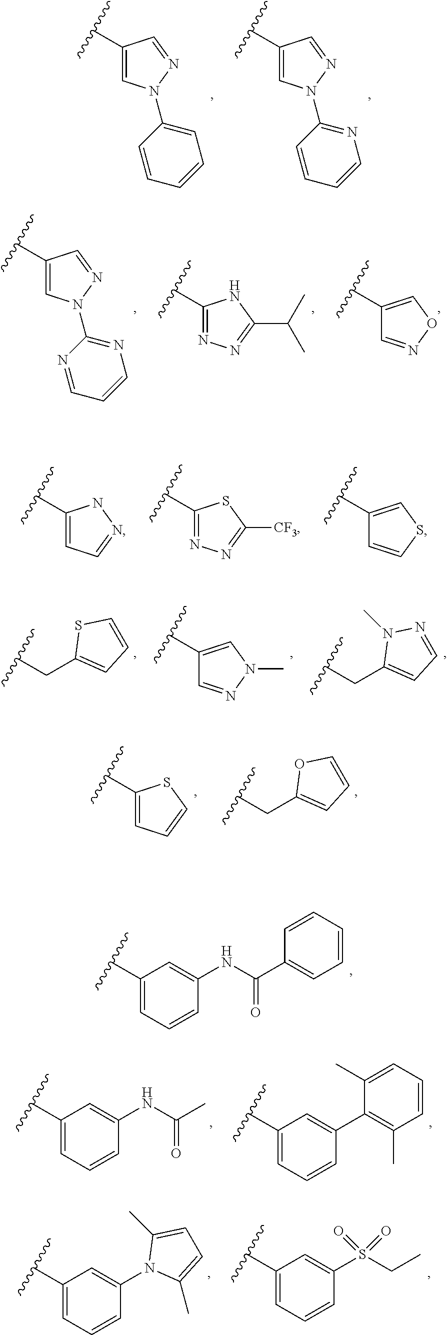

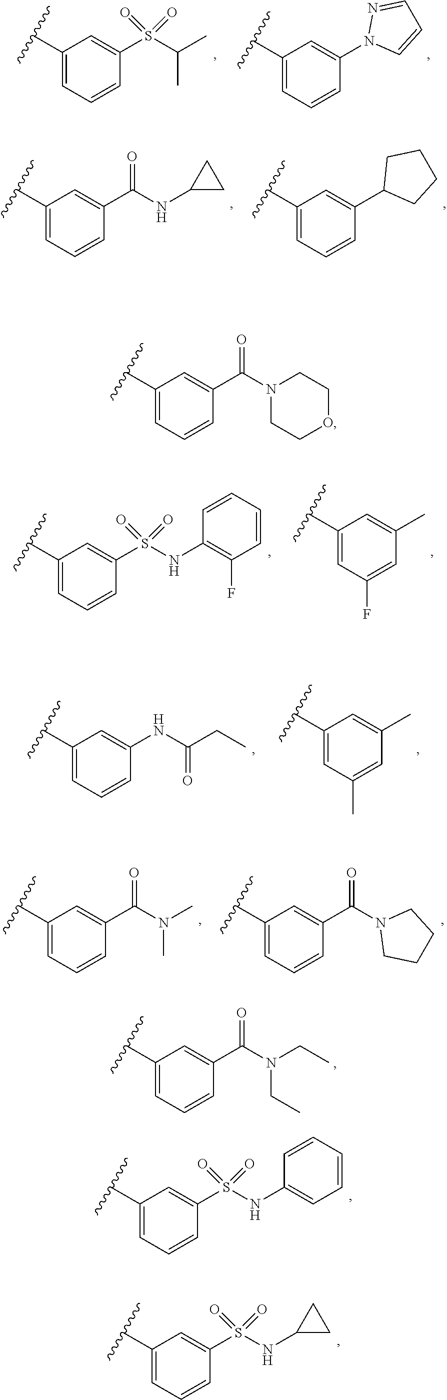

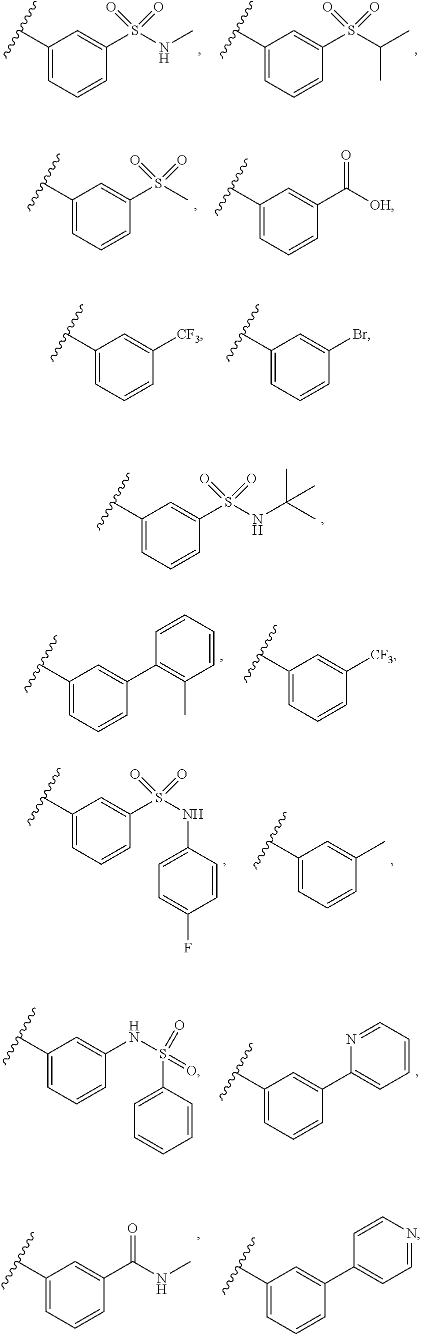

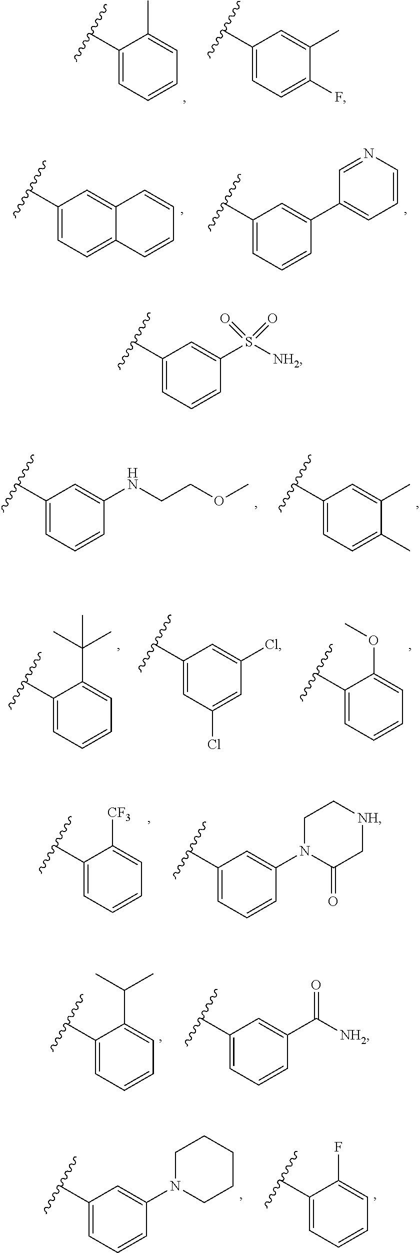

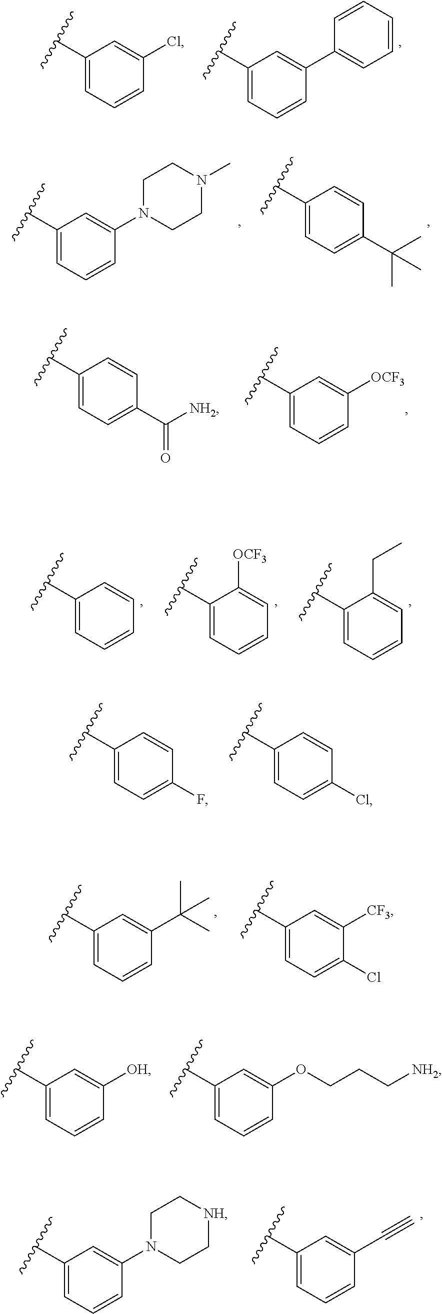

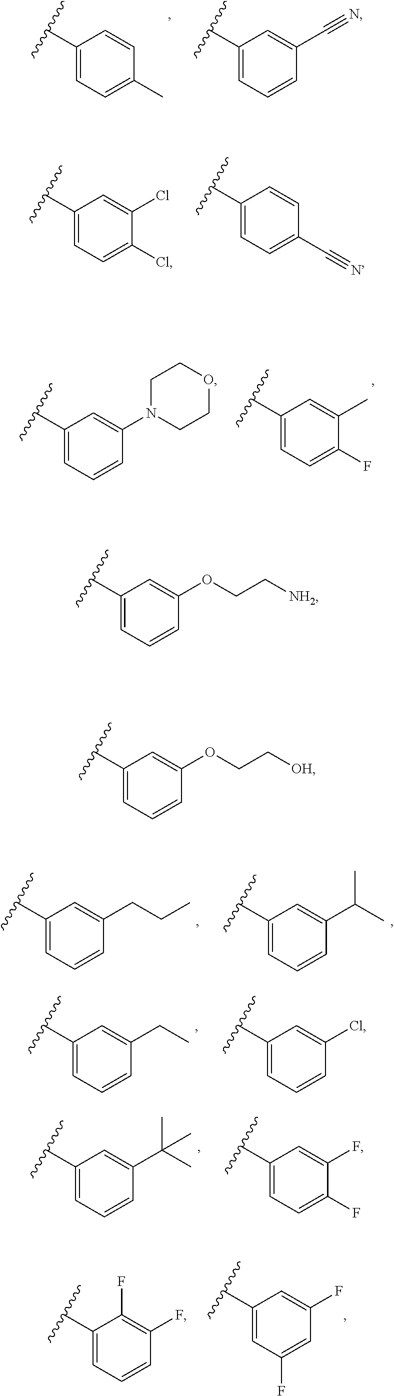

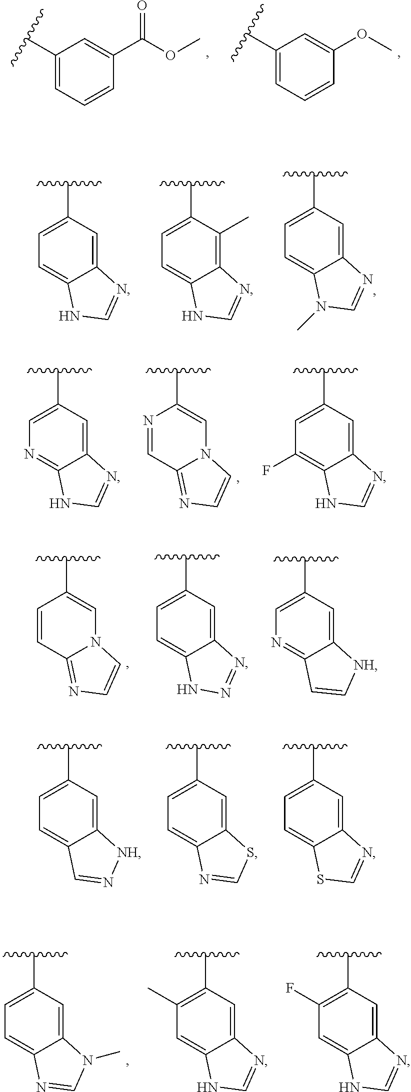

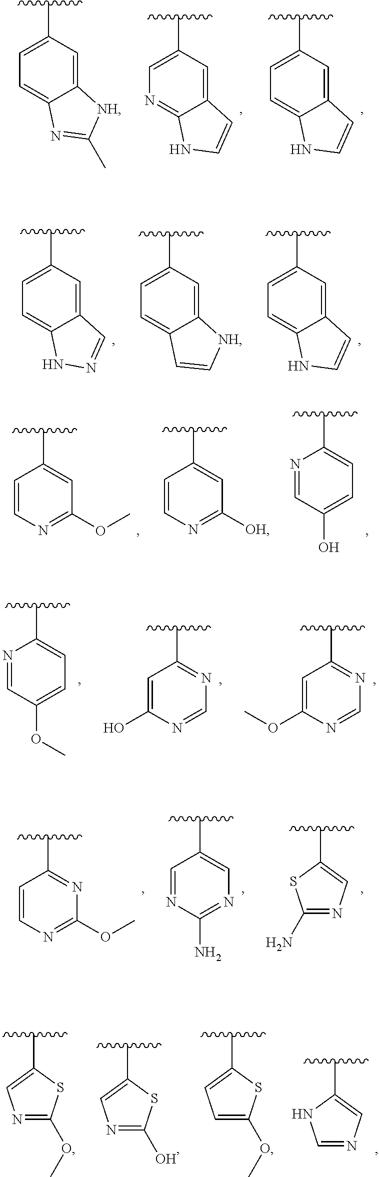

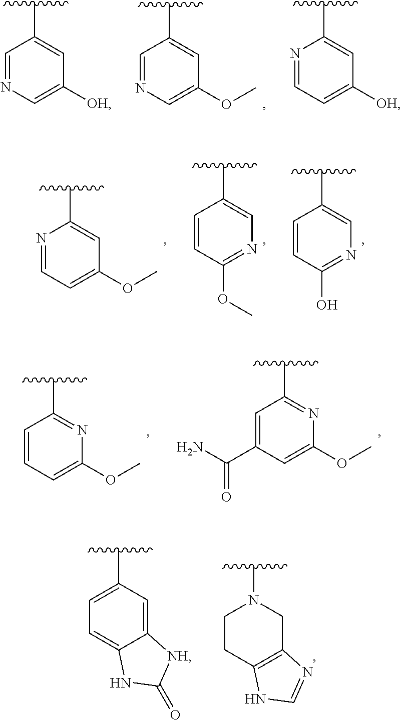

and substitutions thereof.

In some embodiments, R¹ is selected from

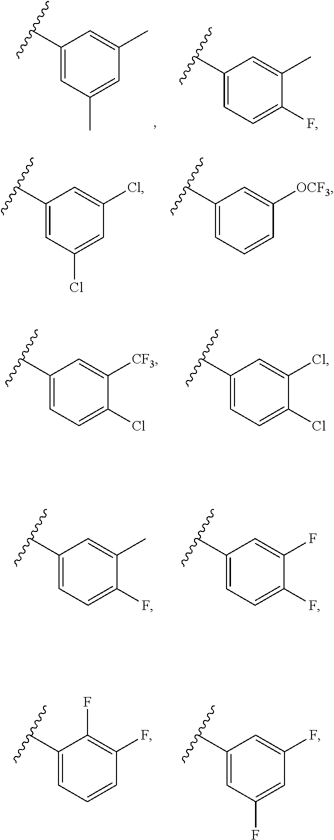

and substitutions thereof.

In some embodiments, R¹ is

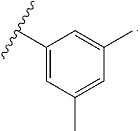 [SMILES: *c1cc(C)cc(C)c1]

In some embodiments, R¹ is

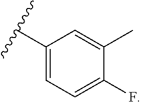 [SMILES: *c1ccc(F)c(C)c1]

In some embodiments, R¹ is

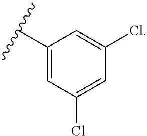 [SMILES: *c1cc(Cl)cc(Cl)c1]

In some embodiments, R¹ is

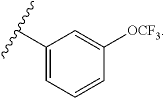 [SMILES: *c1cccc(OC(F)(F)F)c1]

In some embodiments, wherein R¹ is

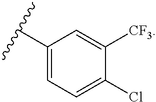 [SMILES: *c1ccc(Cl)c(C(F)(F)F)c1]

In some embodiments, R¹ is

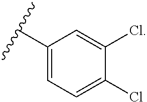 [SMILES: *c1ccc(Cl)c(Cl)c1]

In some embodiments, R¹ is

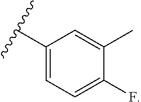 [SMILES: *c1ccc(F)c(C)c1]

In some embodiments, R¹ is

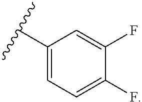 [SMILES: *c1ccc(F)c(F)c1]

In some embodiments, R¹ is F

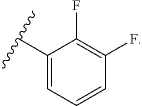 [SMILES: *c1cccc(F)c1F]

In some embodiments, R¹ is

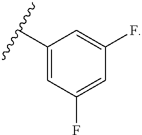 [SMILES: *c1cc(F)cc(F)c1]

In some embodiments, Y is —O—, —S—, —NR₄—, —OC(R⁴)₂—, —SC(R⁴)₂—, —C(R⁴)₂O—, —C(R⁴)₂S—, C(R⁴)₂NR⁴—, —C(R⁴)₂—, or —CR⁴=CR⁴—;

In some embodiments, Y is —O—, —S—, —NR₄—, —OC(R⁴)₂—, —SC(R⁴)₂—, —C(R⁴)₂O—, —C(R⁴)₂S—, —C(R⁴)₂—.

In some embodiments, Y is —O—, —S—, —NH—, —OCH₂—, —SCH₂—, —CH₂O—, —CH₂S—, —CH₂—.

In some embodiments, Y is —O—.

In some embodiments, Y is —S—.

In some embodiments, Y is —S(═O)—.

In some embodiments, Y is —S(═O)₂—.

In some embodiments, Y is —S(═O)C(R⁴)₂—.

In some embodiments, Y is —C(R⁴)₂S(═O)—.

In some embodiments, Y is —S(═O)₂C(R⁴)₂—.

In some embodiments, Y is —C(R⁴)₂S(═O)₂—.

In some embodiments, Y is —S(═O)CH₂—.

In some embodiments, Y is —CH₂S(═O)—.

In some embodiments, Y is —S(═O)₂CH₂—.

In some embodiments, Y is —CH₂S(═O)₂—.

In some embodiments, Y is —NR₄—.

In some embodiments, Y is —OC(R⁴)₂—.

In some embodiments, Y is —SC(R⁴)₂—.

In some embodiments, Y is —C(R⁴)₂O—.

In some embodiments, Y is —C(R⁴)₂S—.

In some embodiments, Y is—C(R⁴)₂NR⁴—.

In some embodiments, Y is —C(R⁴)₂—.

In some embodiments, Y is —C(R⁴)₂—C(R⁴)₂—.

In some embodiments, Y is —CR⁴=CR⁴—;

In some embodiments, Y is —NH—. In some embodiments, Y is —OCH₂—.

In some embodiments, Y is —SCH₂—.

In some embodiments, Y is —CH₂O—.

In some embodiments, Y is —CH₂S—.

In some embodiments, Y is—CH₂NR⁴—.

In some embodiments, Y is —CH₂—.

In some embodiments, Y is —CH₂—CH₂—.

In some embodiments, Y is —CH═CH—.

In some embodiments, Z is —OR⁴, —N(R⁴)₂, —SR⁴, —CF₃, —OCF₃, —OH, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

In some embodiments, Z is —OR⁴, —N(R⁴)₂, —SR⁴, —CF₃, —OCF₃, or is selected from

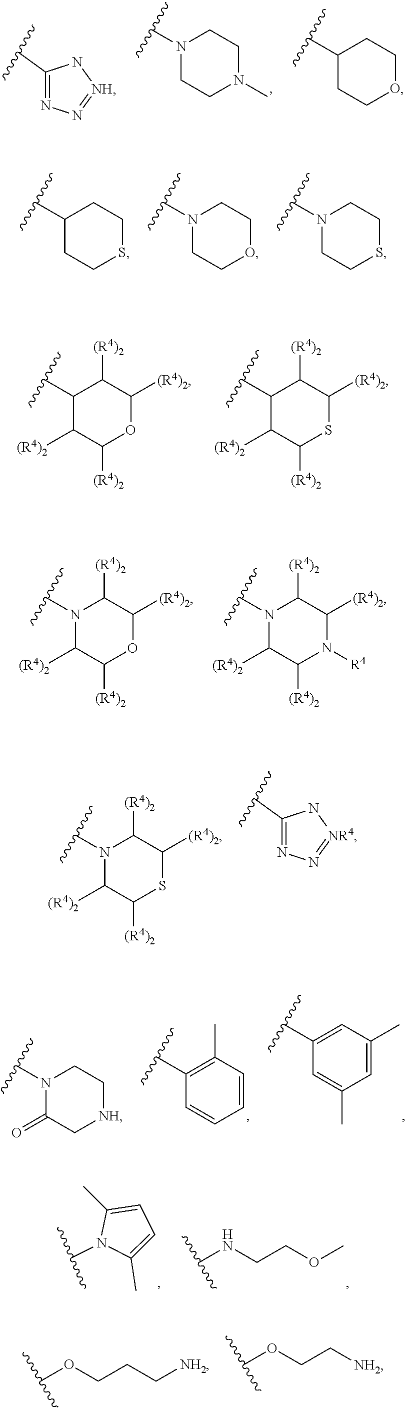

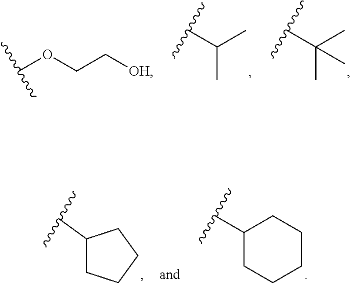

In some embodiments, Z is —OR⁴, —N(R⁴)₂, —SR⁴.

In some embodiments, Z is —OR⁴, —N(R⁴)₂, —SR⁴, and at least one R⁴ of Z is selected from

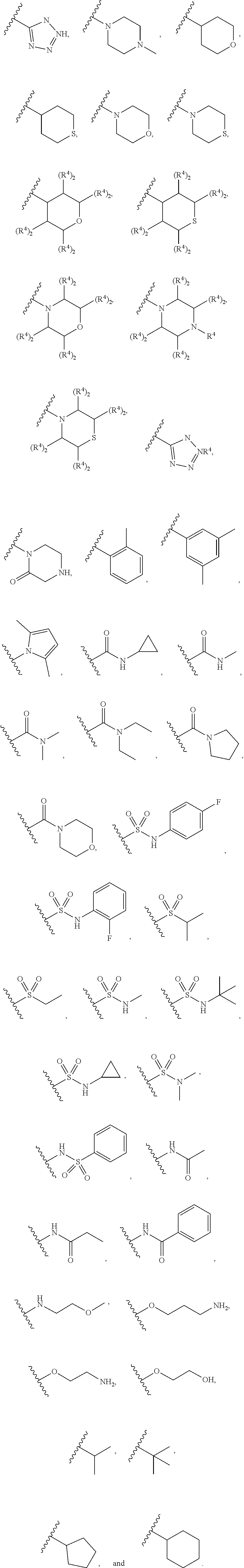

In some embodiments, Z is —OR⁴ or —SR⁴, and the R⁴ of Z is

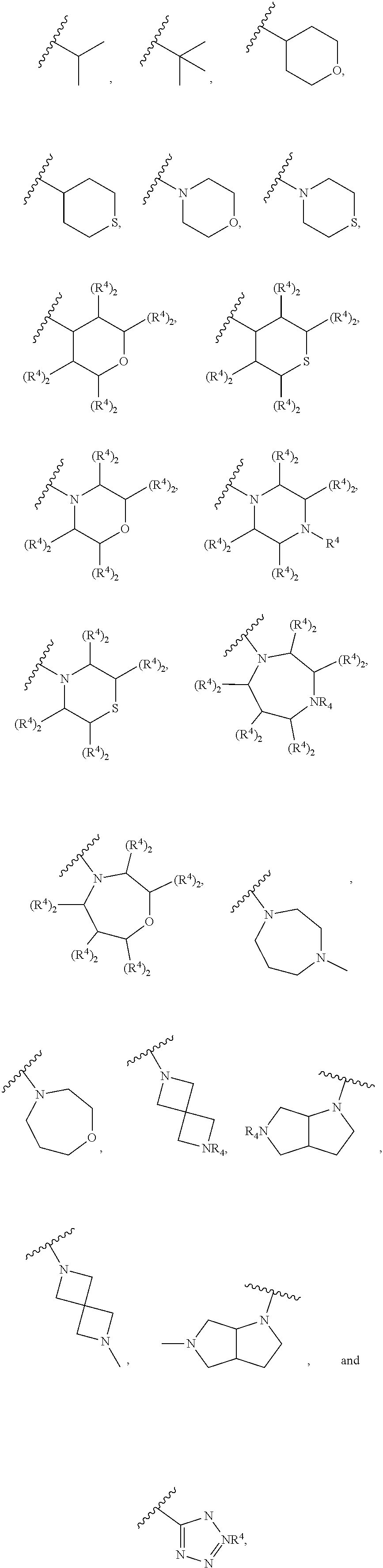

In some embodiments, Z selected from

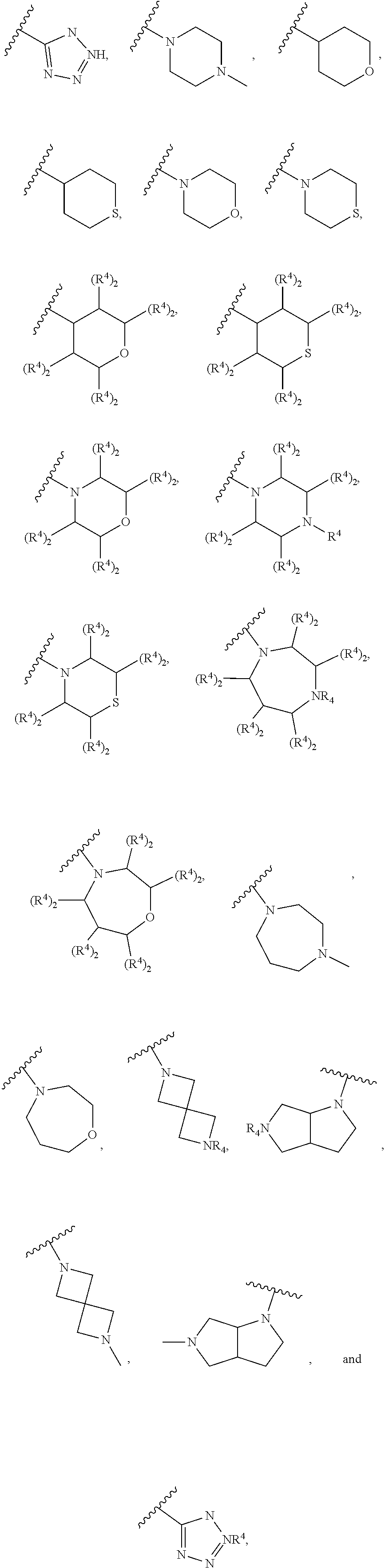

In some embodiments, Z is

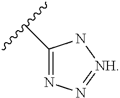

In some embodiments, Z is

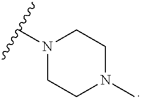 [SMILES: *N1CCN(C)CC1]

In some embodiments, Z is

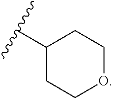 [SMILES: *C1CCOCC1]

In some embodiments, Z is

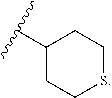 [SMILES: *C1CCSCC1]

In some embodiments, Z is

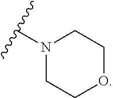 [SMILES: *N1CCOCC1]

In some embodiments, Z is

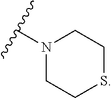 [SMILES: *N1CCSCC1]

In some embodiments, Z is

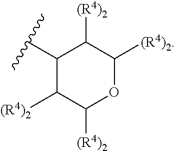

In some embodiments, Z is

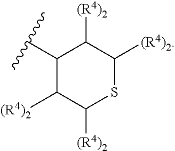

In some embodiments, Z is

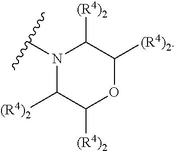

In some embodiments, Z is

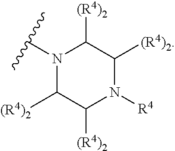

In some embodiments, Z is

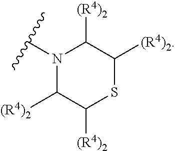

In some embodiments, Z is

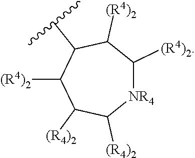

In some embodiments, Z is

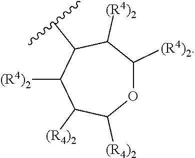

In some embodiments, Z is

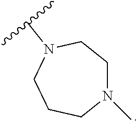 [SMILES: *N1CCCN(C)CC1]

In some embodiments, Z is

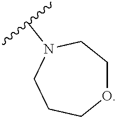 [SMILES: *N1CCCOCC1]

In some embodiments, Z is

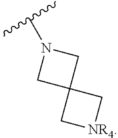

In some embodiments, Z is

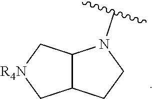

In some embodiments, Z is

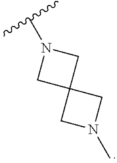 [SMILES: *N1CC2(C1)CN(C)C2]

In some embodiments, Z is

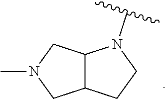 [SMILES: *N1CCC2CN(C)CC21]

In some embodiments, Z is

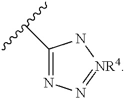

In some embodiments, Z is

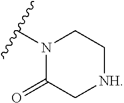 [SMILES: *N1CCNCC1=O]

In some embodiments, Z is

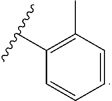 [SMILES: *c1ccccc1C]

In some embodiments, Z is

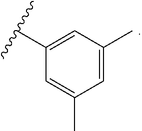 [SMILES: *c1cc(C)cc(C)c1]

In some embodiments, Z is

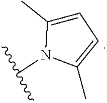 [SMILES: *n1c(C)ccc1C]

In some embodiments, Z is

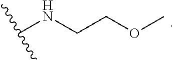 [SMILES: *NCCOC]

In some embodiments, Z is

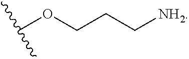 [SMILES: *OCCCN]

In some embodiments, Z is

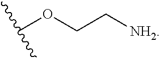 [SMILES: *OCCN]

In some embodiments, Z is

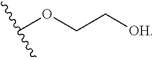 [SMILES: *OCCO]

In some embodiments, Z is

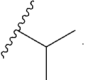 [SMILES: *C(C)C]

In some embodiments, Z is

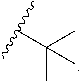 [SMILES: *C(C)(C)C]

In some embodiments, Z is

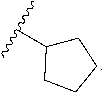 [SMILES: *C1CCCC1]

In some embodiments, Z is

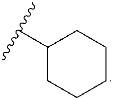 [SMILES: *C1CCCCC1]

In some embodiments, Z is

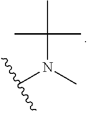 [SMILES: *N(C)C(C)(C)C]

In some embodiments, Z is

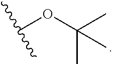 [SMILES: *OC(C)(C)C]

In some embodiments, R¹ or R³ is substituted with halogen.

In some embodiments, R¹ or R³ is substituted with chlorine.

In some embodiments, R¹ or R³ is substituted with fluorine.

In some embodiments, R¹ or R³ is substituted with C₁-C₄ heteroalkyl.

In some embodiments, at least one R⁴ within Z is selected from

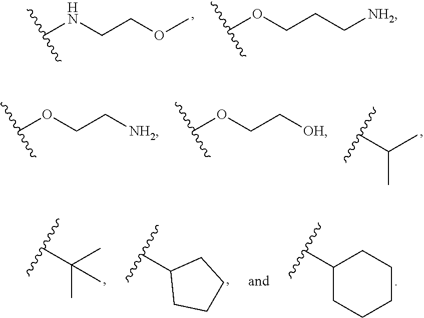

In some embodiments, FSH Modulators disclosed herein have a structure selected from the group of Compound 1-01, Compound 1-02A, Compound 1-02, Compound 1-03, Compound 1-04, Compound 1-05, Compound 1-06, Compound 2-01, Compound 2-02, Compound 2-03, Compound 2-04, Compound 2-05, Compound 2-06, Compound 2-07, Compound 2-08, Compound 3-01, Compound 3-02, Compound 3-03, Compound 3-04, Compound 3-07, Compound 3-08, Compound 3-09, Compound 3-10A, Compound 3-10, Compound 3-11, Compound 3-12, Compound 4-01A, Compound 4-01, Compound 4-02A, Compound 4-02, Compound 4-03A, Compound 4-03, Compound 4-04A,Compound 4-04, Compound 4-05A, Compound 4-05, Compound 4-06A, Compound 4-06, Compound 4-07A, Compound 4-07, Compound 4-08A, Compound 4-08, Compound 5-01, Compound 5-02, Compound 5-03, Compound 5-04, Compound 5-05, Compound 5-06, Compound 5-07, Compound 5-08, Compound 6-01A, Compound 6-01B, Compound 6-01, Compound 6-02A, Compound 6-02B, Compound 6-02, Compound 6-03, Compound 6-04, Compound 6-05, Compound 6-06, Compound 6-07, Compound 6-08, Compound 8-01, Compound 8-02, Compound 8-03, Compound 8-05, Compound 8-06, Compound 8-07A, Compound 8-07, Compound 8-09, Compound 8-10, Compound 8-14, Compound 8-15, Compound 8-16B, Compound 8-16, Compound 8-17, Compound 8-20, Compound 8-21, Compound 8-22, Compound 8-23, Compound 8-24, Compound 8-25, Compound 8-26A, Compound 8-26, Compound 8-27, Compound 8-28, Compound 8-29, Compound 8-30, Compound 8-31, Compound 8-32, Compound 8-33, Compound 8-34, Compound 8-39, and Compound 8-44.

In some embodiments, FSH Modulators disclosed herein have a structure selected from the group of. Compound 8-77, Compound 8-75, Compound 8-76, Compound 8-78, Compound 8-81, Compound 8-61, Compound 8-60, Compound 8-63, Compound 8-58, Compound 8-51, Compound 8-67, Compound 8-74, Compound 8-4, Compound 8-8, Compound 8-4a, Compound 8-13, Compound 8-57, Compound 8-18, Compound 8-35, Compound 8-36, Compound 8-37, Compound 8-38, Compound 8-41, Compound 8-42, Compound 8-43, Compound 8-45, Compound 8-46, Compound 8-47, Compound 8-49, Compound 8-50, Compound 8-52A, Compound 8-54A, Compound 8-55, Compound 8-56, Compound 8-62, Compound 8-64, Compound 8-65, Compound 8-69, Compound 8-70, Compound 8-71, Compound 8-79, Compound 8-82, Compound 8-83, Compound 8-84, Compound 8-86, Compound 8-87, Compound 8-89, Compound 9-13, Compound 9-21, Compound 9-4, Compound 9-5, Compound 9-11, Compound 9-14, Compound 9-9, Compound 9-15, Compound 9-2, Compound 9-7, Compound 9-12, Compound 9-16, Compound 9-17, Compound 9-18, Compound 9-19, Compound 9-20, Compound 10-1, Compound 10-2, Compound 10-3, Compound 10-6, Compound 10-7, Compound 10-8, Compound 10-9, Compound 10-10, Compound 11-1A, Compound 11-2, Compound 11-1, Compound 11-3, Compound 12-2, Compound 12-23, Compound 12-13, Compound 12-15, Compound 12-16, Compound 12-1, Compound 12-4, Compound 12-18, Compound 12-19, Compound 13-1, Compound 13-4, Compound 13-9, Compound 13-7, Compound 13-8, Compound 13-2, Compound 13-5, Compound 15-1, Compound 15-3, Compound 15-4, Compound 15-5, Compound 15-9, Compound 15-2, Compound 15-6, Compound 15-10, Compound 12-05, Compound 12-07, Compound 12-11, Compound 12-12, Compound 14-03, Compound 15-08, Compound 15-10, Compound 3-05, Compound 3-06, Compound 4-03B, Compound 8-04A, Compound 8-16A, Compound 8-23A, Compound 8-25A, Compound 8-26B, Compound 8-31A, Compound 8-33A, Compound 8-44, Compound 8-66, Compound 8-72, Compound 8-90, Compound 8-90A, Compound 9-01, Compound 9-03, Compound 9-06, Compound 9-08, Compound 9-08A, Compound 9-10, Compound 9-19A, and Compound 9-24.

In some cases FSH modulators described herein can have a structure of any of the compounds of Table 1.

TABLE 1
Structures of additional example FSH Modulators

| Compound ID | Structure |
|---|---|
| Compound 12-05 | 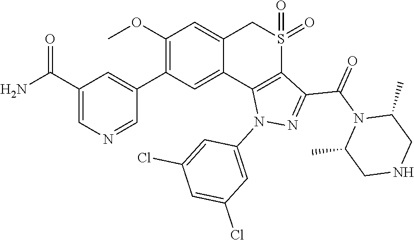 [SMILES: COc1cc2c(cc1-c1cncc(C(N)=O)c1)-c1c(c(C(=O)N3[C@H](C)CNC[C@@H]3C)nn1-c1cc(Cl)cc(Cl)c1)S(=O)(=O)C2] |
| Compound 12-07 | 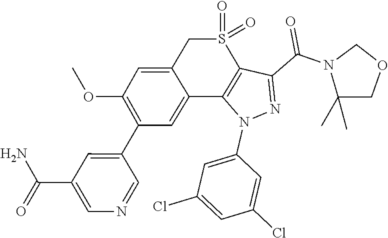 [SMILES: COc1cc2c(cc1-c1cncc(C(N)=O)c1)-c1c(c(C(=O)N3COCC3(C)C)nn1-c1cc(Cl)cc(Cl)c1)S(=O)(=O)C2] |
| Compound 12-11 | 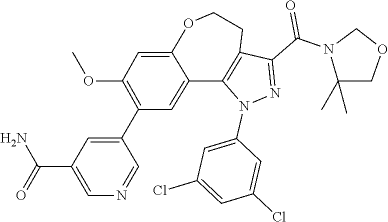 [SMILES: COc1cc2c(cc1-c1cncc(C(N)=O)c1)-c1c(c(C(=O)N3COCC3(C)C)nn1-c1cc(Cl)cc(Cl)c1)CCO2] |
| Compound 12-12 | 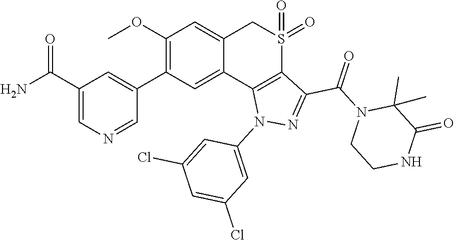 [SMILES: COc1cc2c(cc1-c1cncc(C(N)=O)c1)-c1c(c(C(=O)N3CCNC(=O)C3(C)C)nn1-c1cc(Cl)cc(Cl)c1)S(=O)(=O)C2] |
| Conmpound 14-03 | 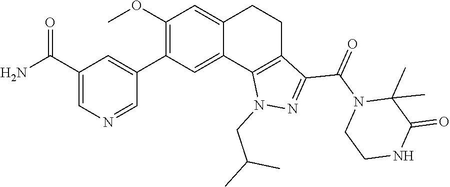 [SMILES: COc1cc2c(cc1-c1cncc(C(N)=O)c1)-c1c(c(C(=O)N3CCNC(=O)C3(C)C)nn1CC(C)C)CC2] |
| Compound 15-08 | 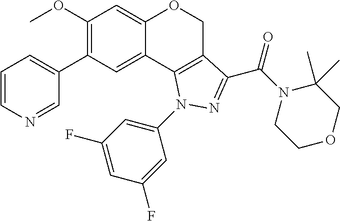 [SMILES: COc1cc2c(cc1-c1cccnc1)-c1c(c(C(=O)N3CCOCC3(C)C)nn1-c1cc(F)cc(F)c1)CO2] |
| Compound 15-10 | 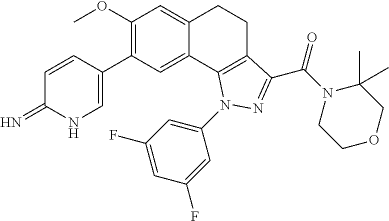 [SMILES: COc1cc2c(cc1-c1ccc(=N)[nH]c1)-c1c(c(C(=O)N3CCOCC3(C)C)nn1-c1cc(F)cc(F)c1)CC2] |
| Compound 3-05 | 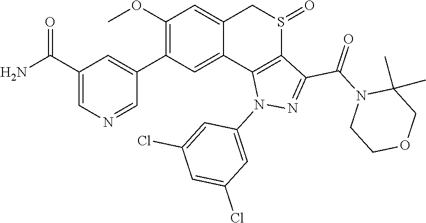 [SMILES: COc1cc2c(cc1-c1cncc(C(N)=O)c1)-c1c(c(C(=O)N3CCOCC3(C)C)nn1-c1cc(Cl)cc(Cl)c1)S(=O)C2] |
| Compound 3-06 | 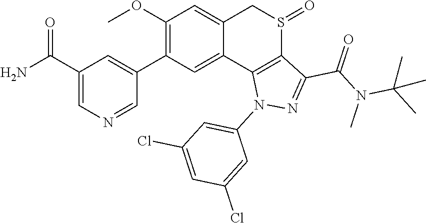 [SMILES: COc1cc2c(cc1-c1cncc(C(N)=O)c1)-c1c(c(C(=O)N(C)C(C)(C)C)nn1-c1cc(Cl)cc(Cl)c1)S(=O)C2] |
| Compound 4-03B | 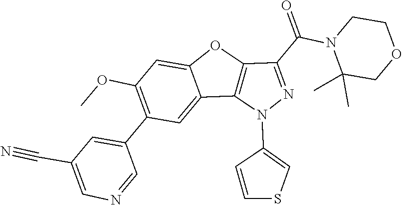 [SMILES: COc1cc2oc3c(C(=O)N4CCOCC4(C)C)nn(-c4ccsc4)c3c2cc1-c1cncc(C#N)c1] |
| Compound 8-04A | 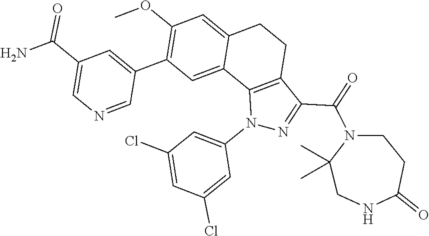 [SMILES: COc1cc2c(cc1-c1cncc(C(N)=O)c1)-c1c(c(C(=O)N3CCC(=O)NCC3(C)C)nn1-c1cc(Cl)cc(Cl)c1)CC2] |
| Compound 8-16A | 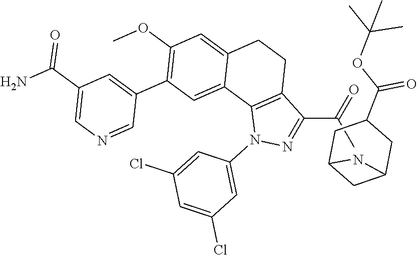 |
| Compound 8-23A | 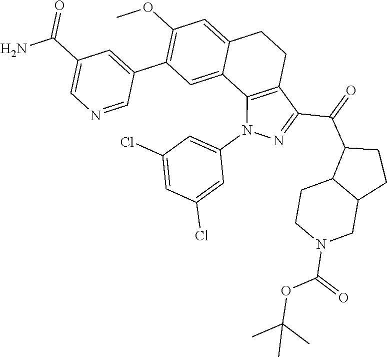 [SMILES: COc1cc2c(cc1-c1cncc(C(N)=O)c1)-c1c(c(C(=O)C3CCC4CN(C(=O)OC(C)(C)C)CCC43)nn1-c1cc(Cl)cc(Cl)c1)CC2] |
| Compound 8-25A | 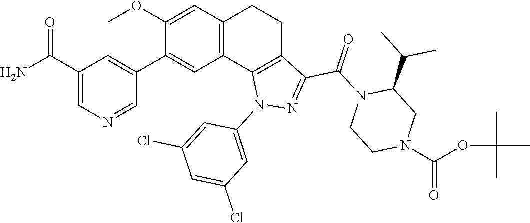 [SMILES: COc1cc2c(cc1-c1cncc(C(N)=O)c1)-c1c(c(C(=O)N3CCN(C(=O)OC(C)(C)C)C[C@@H]3C(C)C)nn1-c1cc(Cl)cc(Cl)c1)CC2] |
| Compound 8-26B | 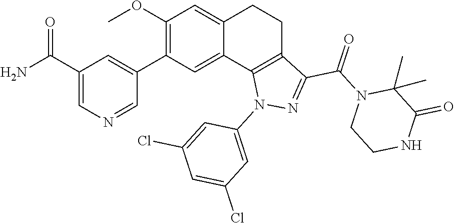 [SMILES: COc1cc2c(cc1-c1cncc(C(N)=O)c1)-c1c(c(C(=O)N3CCNC(=O)C3(C)C)nn1-c1cc(Cl)cc(Cl)c1)CC2] |
| Compound 8-31A | 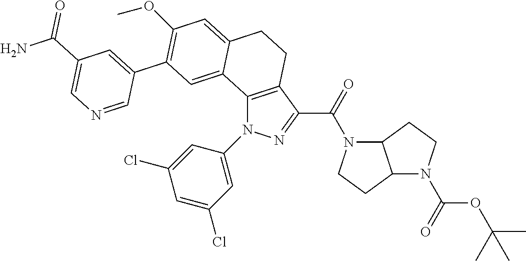 [SMILES: COc1cc2c(cc1-c1cncc(C(N)=O)c1)-c1c(c(C(=O)N3CCC4C3CCN4C(=O)OC(C)(C)C)nn1-c1cc(Cl)cc(Cl)c1)CC2] |
| Compound 8-33A | 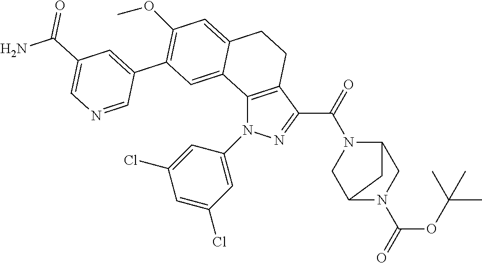 [SMILES: COc1cc2c(cc1-c1cncc(C(N)=O)c1)-c1c(c(C(=O)N3CC4CC3CN4C(=O)OC(C)(C)C)nn1-c1cc(Cl)cc(Cl)c1)CC2] |
| Compound 8-44 | 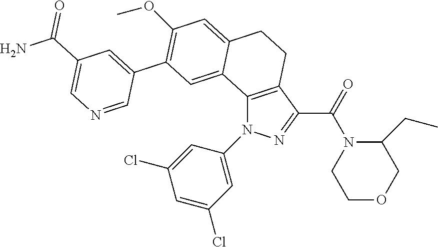 [SMILES: CCC1COCCN1C(=O)c1nn(-c2cc(Cl)cc(Cl)c2)c2c1CCc1cc(OC)c(-c3cncc(C(N)=O)c3)cc1-2] |
| Compound 8-66 | 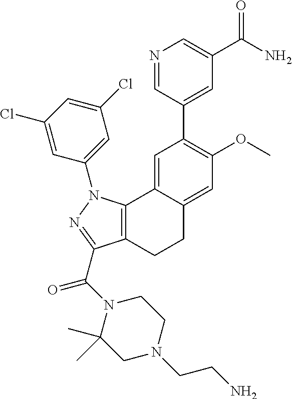 [SMILES: COc1cc2c(cc1-c1cncc(C(N)=O)c1)-c1c(c(C(=O)N3CCN(CCN)CC3(C)C)nn1-c1cc(Cl)cc(Cl)c1)CC2] |
| Compound 8-72 | 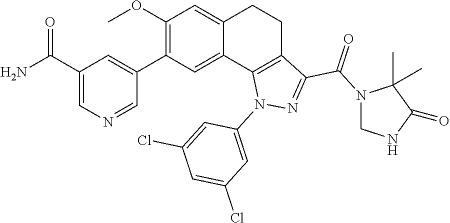 [SMILES: COc1cc2c(cc1-c1cncc(C(N)=O)c1)-c1c(c(C(=O)N3CNC(=O)C3(C)C)nn1-c1cc(Cl)cc(Cl)c1)CC2] |
| Compound 8-90 | 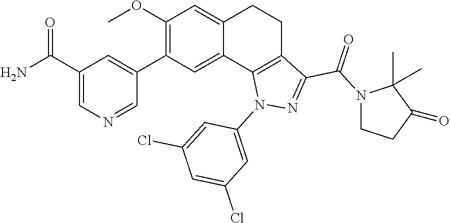 [SMILES: COc1cc2c(cc1-c1cncc(C(N)=O)c1)-c1c(c(C(=O)N3CCC(=O)C3(C)C)nn1-c1cc(Cl)cc(Cl)c1)CC2] |
| Compound 8-90A | 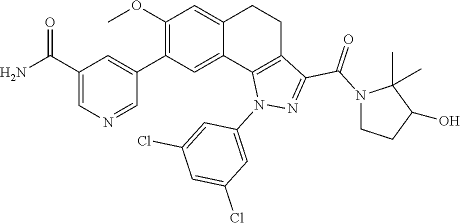 [SMILES: COc1cc2c(cc1-c1cncc(C(N)=O)c1)-c1c(c(C(=O)N3CCC(O)C3(C)C)nn1-c1cc(Cl)cc(Cl)c1)CC2] |
| Compound 9-01 | 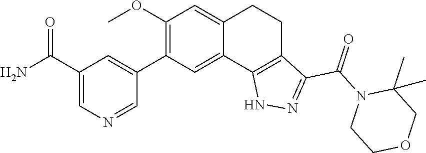 [SMILES: COc1cc2c(cc1-c1cncc(C(N)=O)c1)-c1[nH]nc(C(=O)N3CCOCC3(C)C)c1CC2] |
| Compound 9-03 | 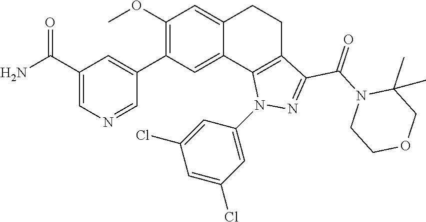 [SMILES: COc1cc2c(cc1-c1cncc(C(N)=O)c1)-c1c(c(C(=O)N3CCOCC3(C)C)nn1-c1cc(Cl)cc(Cl)c1)CC2] |
| Compound 9-06 | 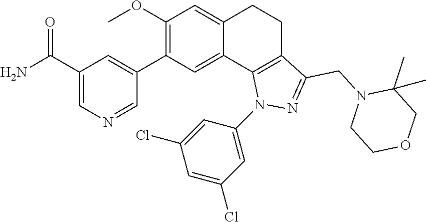 [SMILES: COc1cc2c(cc1-c1cncc(C(N)=O)c1)-c1c(c(CN3CCOCC3(C)C)nn1-c1cc(Cl)cc(Cl)c1)CC2] |
| Compound 9-08 | 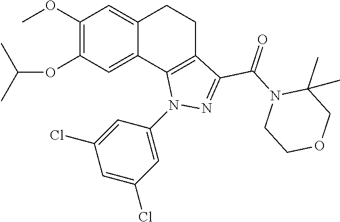 [SMILES: COc1cc2c(cc1OC(C)C)-c1c(c(C(=O)N3CCOCC3(C)C)nn1-c1cc(Cl)cc(Cl)c1)CC2] |
| Compound 9-08A | 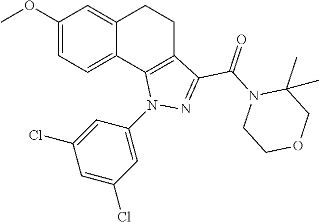 |
| Compound 9-10 | 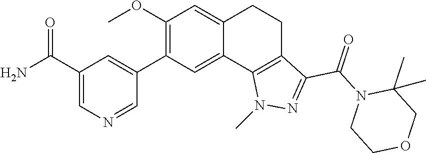 [SMILES: COc1cc2c(cc1-c1cncc(C(N)=O)c1)-c1c(c(C(=O)N3CCOCC3(C)C)nn1C)CC2] |
| Compound 9-19A | 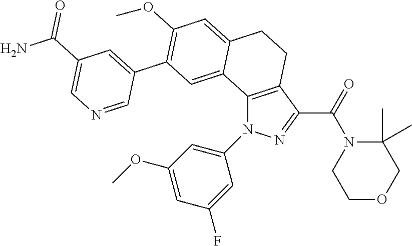 [SMILES: COc1cc(F)cc(-n2nc(C(=O)N3CCOCC3(C)C)c3c2-c2cc(-c4cncc(C(N)=O)c4)c(OC)cc2CC3)c1] |
| Compound 9-24 | 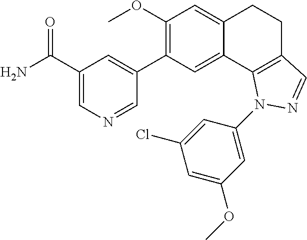 [SMILES: COc1cc(Cl)cc(-n2ncc3c2-c2cc(-c4cncc(C(N)=O)c4)c(OC)cc2CC3)c1] |

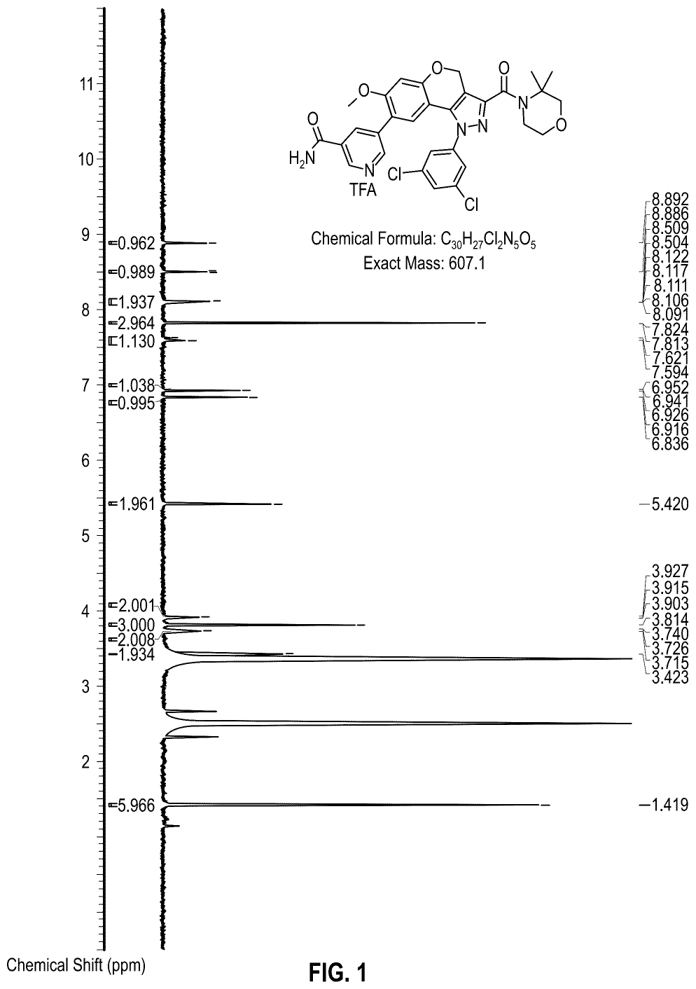

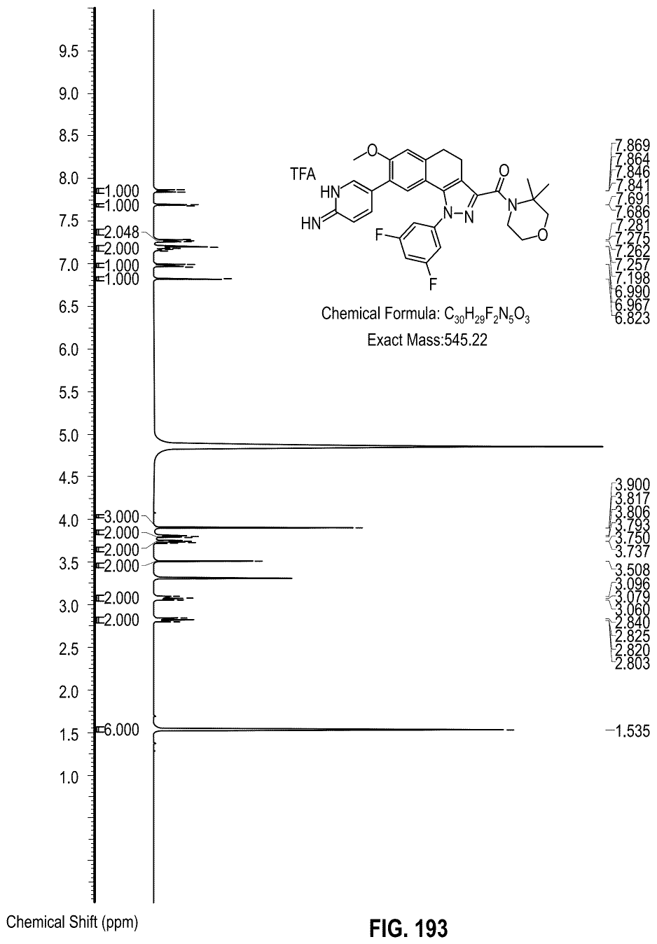

In some instances, FSH modulators described herein can have any structure described herein, including, but not limited to, those described in FIGS. 1-193, in the Examples, and/or the synthetic schemes throughout the instant application, and pharmaceutically acceptable salts, solvates, or formulations thereof.

In another aspect, described herein are methods of modulating FSH using compounds described herein. In some embodiments, compounds described herein selectively modulate FSH and do not substantially modulate TSH. In some embodiments, the methods comprise administering a compound described herein to a subject. In some embodiments, compounds described herein are FSH agonists. In some embodiments, compounds described herein are selective by at least 3-fold for FSH over TSH (e.g. at least 3, 5, 10, 20, 50, or 100 fold). In some embodiments, an in-vitro or in-vivo EC₅₀ for FSH agonism is no more than about 100 nM (e.g. no more than 100 nM, 50 nM, 10 nM, 5 nM, 1 nM, or 500 pM).

Any of the methods described herein can comprise treating a disease or condition comprising administering any of the compounds described herein (or a pharmaceutically acceptable salt or solvate thereof) to a subject in need thereof. In some embodiments, the disease or condition is a fertility disorder or male hypogonadism. In some embodiments, the disease or condition is cancer. In some embodiments, the cancer is breast, prostate, colon, pancreas, urinary bladder, kidney, lung, liver, stomach, testicular, or ovarian cancer. In some embodiments, the disease or condition is a cardiovascular condition. In some embodiments, the cardiovascular condition is atherosclerosis. In some embodiments, the disease or condition is a body composition disorder (e.g. obesity). In some embodiments, the disease or condition is non-alcoholic fatty liver disease. In some embodiments, the disease or condition is a bone density disorder (e.g. osteoporosis). In some embodiments, the disease or condition is Turner syndrome, Klinefelter syndrome, polycystic ovary syndrome (PCOS), and/or primary ovary insufficiency (POI).

In some embodiments, the disease or condition is polycystic ovary syndrome (PCOS).

In some embodiments, the disease or condition is Turner syndrome.

In some embodiments, the disease or condition is Klinefelter syndrome.

In some embodiments, the disease or condition is primary ovary insufficiency (POI).

Further described herein described herein are pharmaceutical compositions comprising a compound of any one of claims or a pharmaceutically acceptable salt or pharmaceutically acceptable solvate thereof, and a pharmaceutically acceptable excipient or carrier.

Pharmaceutically acceptable lipid nanoparticle formulation can comprise any of the compounds described herein.

Any of the methods described herein can comprise treating a condition or disease by administering any compound, pharmaceutically acceptable salt, and/or pharmaceutically acceptable solvate described herein to a subject in need thereof.

Any of the methods described herein can comprise use of any compound, pharmaceutically acceptable salt, and/or pharmaceutically acceptable solvate described herein in the manufacture of a medicament for the treatment of a condition or disease.

The compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

Modulation of FSHR. or a mutant thereof, activity in a biological sample is useful for a variety of purposes that are known to one of skill in the art. Examples of such purposes include, but are not limited to, blood transfusion, organ transplantation. biological specimen storage, and biological assays.

### Further Forms of Compounds

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures may reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other methods, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

It is understood that other analogous procedures and reagents could be used, and that these Schemes are only meant as non-limiting examples.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin. In another aspect, the compounds disclosed herein are administered directly to the reproductive tract of women (vaginal gel, vaginal ring, intrauterine delivery) using non-degradable or degradable delivery systems. In another aspect, the compounds disclosed herein are administered directly to the reproductive tract of men using non-degradable or degradable delivery systems.

In another aspect, the compounds disclosed herein are administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned aspects are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

In some embodiments, the dose is about 0.1 mg per day to about 5,000 mg per day. In some embodiments, the dose is about 0.1 mg per day to about 1 mg per day, about 0.1 mg per day to about 50 mg per day, about 0.1 mg per day to about 100 mg per day, about 0.1 mg per day to about 300 mg per day, about 0.1 mg per day to about 500 mg per day, about 0.1 mg per day to about 600 mg per day, about 0.1 mg per day to about 700 mg per day, about 0.1 mg per day to about 800 mg per day, about 0.1 mg per day to about 900 mg per day, about 0.1 mg per day to about 1,000 mg per day, about 0.1 mg per day to about 5,000 mg per day, about 1 mg per day to about 50 mg per day, about 1 mg per day to about 100 mg per day, about 1 mg per day to about 300 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 1 mg per day to about 5,000 mg per day, about 50 mg per day to about 100 mg per day, about 50 mg per day to about 300 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 50 mg per day to about 5,000 mg per day, about 100 mg per day to about 300 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 100 mg per day to about 5,000 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 300 mg per day to about 5,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 500 mg per day to about 5,000 mg per day, about 600 mg per day to about 700 mg per day, about 600 mg per day to about 800 mg per day, about 600 mg per day to about 900 mg per day, about 600 mg per day to about 1,000 mg per day, about 600 mg per day to about 5,000 mg per day, about 700 mg per day to about 800 mg per day, about 700 mg per day to about 900 mg per day, about 700 mg per day to about 1,000 mg per day, about 700 mg per day to about 5,000 mg per day, about 800 mg per day to about 900 mg per day, about 800 mg per day to about 1,000 mg per day, about 800 mg per day to about 5,000 mg per day, about 900 mg per day to about 1,000 mg per day, about 900 mg per day to about 5,000 mg per day, or about 1,000 mg per day to about 5,000 mg per day. In some embodiments, the dose is about 0.1 mg per day, about 1 mg per day, about 50 mg per day, about 100 mg per day, about 300 mg per day, about 500 mg per day, about 600 mg per day, about 700 mg per day, about 800 mg per day, about 900 mg per day, about 1,000 mg per day, or about 5,000 mg per day. In some embodiments, the dose is at least about 0.1 mg per day, about 1 mg per day, about 50 mg per day, about 100 mg per day, about 300 mg per day, about 500 mg per day, about 600 mg per day, about 700 mg per day, about 800 mg per day, about 900 mg per day, or about 1,000 mg per day. In some embodiments, the dose is at most about 1 mg per day, about 50 mg per day, about 100 mg per day, about 300 mg per day, about 500 mg per day, about 600 mg per day, about 700 mg per day, about 800 mg per day, about 900 mg per day, about 1,000 mg per day, or about 5,000 mg per day.

In some embodiments, the dose is about 1 mg per day to about 1,000 mg per day. In some embodiments, the dose is about 1 mg per day to about 50 mg per day, about 1 mg per day to about 100 mg per day, about 1 mg per day to about 200 mg per day, about 1 mg per day to about 300 mg per day, about 1 mg per day to about 400 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 50 mg per day to about 100 mg per day, about 50 mg per day to about 200 mg per day, about 50 mg per day to about 300 mg per day, about 50 mg per day to about 400 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 100 mg per day to about 200 mg per day, about 100 mg per day to about 300 mg per day, about 100 mg per day to about 400 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 200 mg per day to about 300 mg per day, about 200 mg per day to about 400 mg per day, about 200 mg per day to about 500 mg per day, about 200 mg per day to about 600 mg per day, about 200 mg per day to about 700 mg per day, about 200 mg per day to about 800 mg per day, about 200 mg per day to about 900 mg per day, about 200 mg per day to about 1,000 mg per day, about 300 mg per day to about 400 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 400 mg per day to about 500 mg per day, about 400 mg per day to about 600 mg per day, about 400 mg per day to about 700 mg per day, about 400 mg per day to about 800 mg per day, about 400 mg per day to about 900 mg per day, about 400 mg per day to about 1,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 600 mg per day to about 700 mg per day, about 600 mg per day to about 800 mg per day, about 600 mg per day to about 900 mg per day, about 600 mg per day to about 1,000 mg per day, about 700 mg per day to about 800 mg per day, about 700 mg per day to about 900 mg per day, about 700 mg per day to about 1,000 mg per day, about 800 mg per day to about 900 mg per day, about 800 mg per day to about 1,000 mg per day, or about 900 mg per day to about 1,000 mg per day. In some embodiments, the dose is about 1 mg per day, about 50 mg per day, about 100 mg per day, about 200 mg per day, about 300 mg per day, about 400 mg per day, about 500 mg per day, about 600 mg per day, about 700 mg per day, about 800 mg per day, about 900 mg per day, or about 1,000 mg per day. In some embodiments, the dose is at least about 1 mg per day, about 50 mg per day, about 100 mg per day, about 200 mg per day, about 300 mg per day, about 400 mg per day, about 500 mg per day, about 600 mg per day, about 700 mg per day, about 800 mg per day, or about 900 mg per day. In some embodiments, the dose is at most about 50 mg per day, about 100 mg per day, about 200 mg per day, about 300 mg per day, about 400 mg per day, about 500 mg per day, about 600 mg per day, about 700 mg per day, about 800 mg per day, about 900 mg per day, or about 1,000 mg per day.

In some embodiments, the dose is about 0.1 mg/kg to about 200 mg/kg. In some embodiments, the dose is about 0.1 mg/kg to about 1 mg/kg, about 0.1 mg/kg to about 3 mg/kg, about 0.1 mg/kg to about 5 mg/kg, about 0.1 mg/kg to about 10 mg/kg, about 0.1 mg/kg to about 50 mg/kg, about 0.1 mg/kg to about 70 mg/kg, about 0.1 mg/kg to about 90 mg/kg, about 0.1 mg/kg to about 120 mg/kg, about 0.1 mg/kg to about 150 mg/kg, about 0.1 mg/kg to about 200 mg/kg, about 1 mg/kg to about 3 mg/kg, about 1 mg/kg to about 5 mg/kg, about 1 mg/kg to about 10 mg/kg, about 1 mg/kg to about 50 mg/kg, about 1 mg/kg to about 70 mg/kg, about 1 mg/kg to about 90 mg/kg, about 1 mg/kg to about 120 mg/kg, about 1 mg/kg to about 150 mg/kg, about 1 mg/kg to about 200 mg/kg, about 3 mg/kg to about 5 mg/kg, about 3 mg/kg to about 10 mg/kg, about 3 mg/kg to about 50 mg/kg, about 3 mg/kg to about 70 mg/kg, about 3 mg/kg to about 90 mg/kg, about 3 mg/kg to about 120 mg/kg, about 3 mg/kg to about 150 mg/kg, about 3 mg/kg to about 200 mg/kg, about 5 mg/kg to about 10 mg/kg, about 5 mg/kg to about 50 mg/kg, about 5 mg/kg to about 70 mg/kg, about 5 mg/kg to about 90 mg/kg, about 5 mg/kg to about 120 mg/kg, about 5 mg/kg to about 150 mg/kg, about 5 mg/kg to about 200 mg/kg, about 10 mg/kg to about 50 mg/kg, about 10 mg/kg to about 70 mg/kg, about 10 mg/kg to about 90 mg/kg, about 10 mg/kg to about 120 mg/kg, about 10 mg/kg to about 150 mg/kg, about 10 mg/kg to about 200 mg/kg, about 50 mg/kg to about 70 mg/kg, about 50 mg/kg to about 90 mg/kg, about 50 mg/kg to about 120 mg/kg, about 50 mg/kg to about 150 mg/kg, about 50 mg/kg to about 200 mg/kg, about 70 mg/kg to about 90 mg/kg, about 70 mg/kg to about 120 mg/kg, about 70 mg/kg to about 150 mg/kg, about 70 mg/kg to about 200 mg/kg, about 90 mg/kg to about 120 mg/kg, about 90 mg/kg to about 150 mg/kg, about 90 mg/kg to about 200 mg/kg, about 120 mg/kg to about 150 mg/kg, about 120 mg/kg to about 200 mg/kg, or about 150 mg/kg to about 200 mg/kg. In some embodiments, the dose is about 0.1 mg/kg, about 1 mg/kg, about 3 mg/kg, about 5 mg/kg, about 10 mg/kg, about 50 mg/kg, about 70 mg/kg, about 90 mg/kg, about 120 mg/kg, about 150 mg/kg, or about 200 mg/kg. In some embodiments, the dose is at least about 0.1 mg/kg, about 1 mg/kg, about 3 mg/kg, about 5 mg/kg, about 10 mg/kg, about 50 mg/kg, about 70 mg/kg, about 90 mg/kg, about 120 mg/kg, or about 150 mg/kg. In some embodiments, the dose is at most about 1 mg/kg, about 3 mg/kg, about 5 mg/kg, about 10 mg/kg, about 50 mg/kg, about 70 mg/kg, about 90 mg/kg, about 120 mg/kg, about 150 mg/kg, or about 200 mg/kg.

### EXAMPLES

The following examples are included for illustrative purposes only and are not intended to limit the scope of the disclosure.

### Chemical Synthesis

All reactions were performed in oven-dried glassware. NMR were performed on a 400 MHz Bruker.

### Example 1

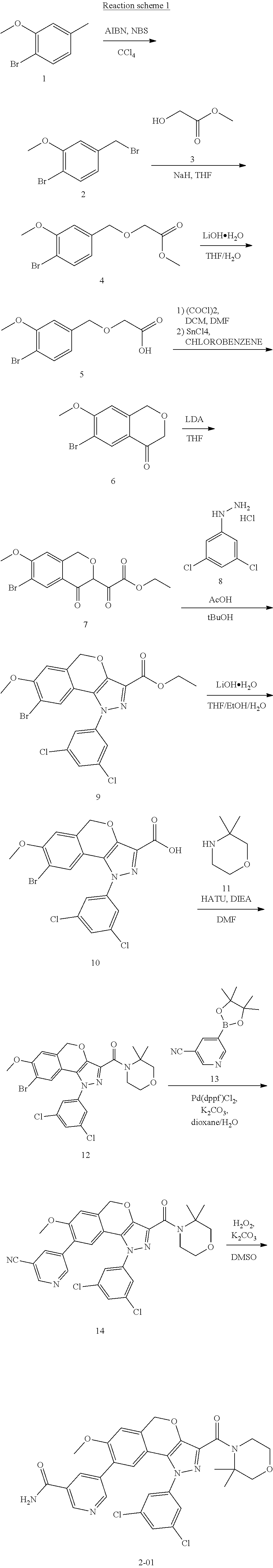

### Synthesis of 1-bromo-4-(bromomethyl)-2-methoxy-benzene

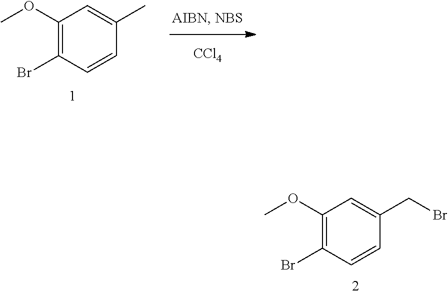

To a mixture of 1-bromo-2-methoxy-4-methyl-benzene (10 g, 49.74 mmol, 1 eq) in CCl4 (100 mL) was added AIBN (816.71 mg, 4.97 mmol, 0.1 eq), and NBS (9.29 g, 52.22 mmol, 1.05 eq), the mixture was stirred at 90° C. for 16 h. The reaction mixture was concentrated under reduced pressure to give a residue. The residue was purified by flash silica gel chromatography (ISCO®; 80 g SepaFlash® Silica Flash Column, Eluent of 0-10% Ethyl acetate/Petroleum ether gradient A 60 mL/min). Compound 1-bromo-4-(bromomethyl)-2-methoxy-benzene (13.9 g, 49.65 mmol, 99% yield) was obtained as a yellow oil. 1H NMR (400 MHz, CHLOROFORM-d) δ=7.50 (d, J=8.0 Hz, 1H), 6.93 (s, 1H), 6.88 (br d, J=8.0 Hz, 1H), 4.46 (s, 2H), 3.92 (s, 3H).

### Synthesis of methyl 2-[(4-bromo-3-methoxy-phenyl)methoxy]acetate

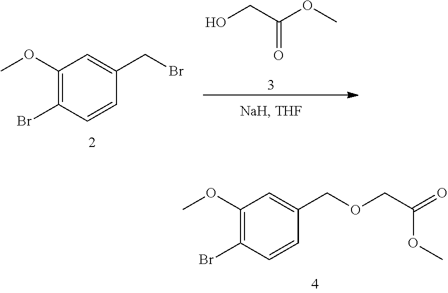

To a mixture of methyl 2-hydroxyacetate (4.50 g, 50.01 mmol, 3.86 mL, 2 eq) in THF (50 mL) was added NaH (2.10 g, 52.51 mmol, 60% purity, 2.1 eq), the mixture was stirred at 25° C. for 15 min, then a mixture of 1-bromo-4-(bromomethyl)-2-methoxy-benzene (7 g, 25.00 mmol, 1 eq) in THF (25 mL) was added, the mixture was stirred at 20-40° C. for 34 h. The reaction mixture was quenched with sat.NH4Cl (100 mL), extracted with EtOAc (100 mL*3). The combined organic phase were washed with brine (100 mL*1), dried over anhydrous Na2SO4, It was filtered and the filtrate was concentrated under reduced pressure to give a residue. The residue was purified by flash silica gel chromatography (ISCO®; 80 g SepaFlash® Silica Flash Column, Eluent of 0-11% Ethyl acetate/Petroleum ether gradient @60 mL/min). Compound methyl 2-[(4-bromo-3-methoxy-phenyl)methoxy]acetate (3.88 g, 13.42 mmol, 54% yield) was obtained as a yellow oil. 1H NMR (400 MHz, CHLOROFORM-d) δ=7.51 (d, J=8.0 Hz, 1H), 6.97 (d, J=1.6 Hz, 1H), 6.82 (dd, J=1.6, 8.0 Hz, 1H), 4.60 (s, 2H), 4.13 (s, 2H), 3.92 (s, 3H), 3.78 (s, 3H).

### Synthesis of 2-[(4-bromo-3-methoxy-phenyl)methoxy]acetic acid

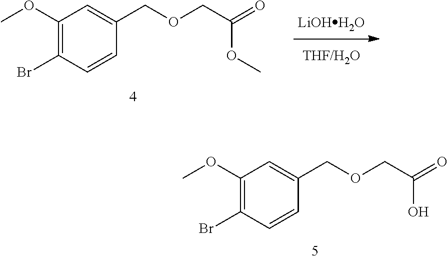

To a solution of methyl 2-[(4-bromo-3-methoxy-phenyl)methoxy]acetate (3.68 g, 12.73 mmol, 1 eq) in THF (20 mL) was added a solution of LiOH. H2O (2.14 g, 50.91 mmol, 4 eq) in H2O (20 mL). The mixture was stirred at 20° C. for 16 hr. The reaction mixture was acidified to pH=6 with 1 M HCl aqueous solution, There were precipitates formed in the reaction mixture. The mixture was filtered and the filter cake was collected and dried in vacuo. Compound 2-[(4-bromo-3-methoxy-phenyl)methoxy]acetic acid (2.9 g, 10.54 mmol, 83% yield) was obtained as a yellow solid. 1H NMR (400 MHz, DMSO-d6) δ=12.70 (br s, 1H), 7.53 (d, J=8.0 Hz, 1H), 7.08 (d, J=1.2 Hz, 1H), 6.87 (d, J=8.0 Hz, 1H), 4.52 (s, 2H), 4.08 (s, 2H), 3.84 (s, 3H).

### Synthesis of 6-bromo-7-methoxy-isochroman-4-one

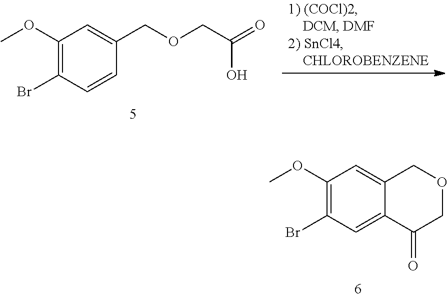

To a solution of 2-[(4-bromo-3-methoxy-phenyl)methoxy]acetic acid (2.5 g, 9.09 mmol, 1 eq) in DCM (25 mL) was added (COCl)₂ (1.79 g, 14.09 mmol, 1.23 mL, 1.55 eq) and DMF (0.1 mL) at 0° C. The mixture was stirred at 20° C. for 0.5 hr. The reaction mixture was concentrated under reduced pressure to give a residue. Compound 2-[(4-bromo-3-methoxy-phenyl) methoxy]acetyl chloride (2.67 g, crude) was obtained as a yellow oil.

To a solution of 2-[(4-bromo-3-methoxy-phenyl) methoxy]acetyl chloride (2.67 g, 9.10 mmol, 1 eq) in chlorobenzene (25 mL) was added tetrachlorostanane (1 M, 18.65 mL, 2.05 eq) at 0° C. under N₂ atmosphere. The mixture was stirred at 0° C. for 0.5 hr. The reaction mixture was quenched with sat.NaHCO₃ (100 mL) and H₂O (100 mL), extracted with EtOAc (200 mL*3). The combined organic phases were washed with brine (200 mL), dried over anhydrous Na₂SO₄, The mixture was filtered and the filtrate was concentrated under reduced pressure to provide a residue. The residue was purified by flash silica gel chromatography (ISCO®; 40 g SepaFlash® Silica Flash Column, Eluent of 0˜20% Ethyl acetate/Petroleum ether gradient @50 mL/min). Compound 6-bromo-7-methoxy-isochroman-4-one (1.08 g, 4.20 mmol, 46% yield) was obtained as a yellow solid. ¹H NMR (400 MHz, CHLOROFORM-d) δ=8.25 (s, 1H), 6.67 (s, 1H), 4.85 (s, 2H), 4.33 (s, 2H), 3.98 (s, 3H).

### Synthesis of Ethyl 2-(6-bromo-7-methoxy-4-oxo-isochroman-3-yl)-2-oxo-acetate

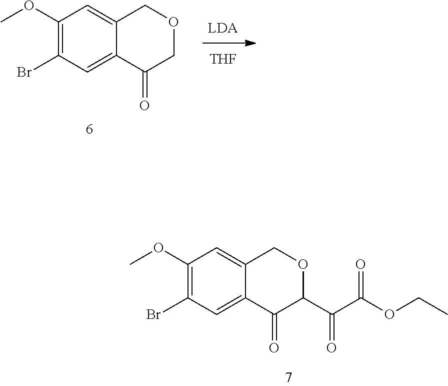 [SMILES: COc1cc2c(cc1Br)C(=O)COC2 ; "THF" ; CCOC(=O)C(=O)C1OCc2cc(OC)c(Br)cc2C1=O]

To a solution of N-isopropylpropan-2-amine (393.61 mg, 3.89 mmol, 549.74 μL, 2 eq) in THF (10 mL) was added n-BuLi (2.5 M, 1.01 mL, 1.3 eq) at −78° C. in dropwise under N₂ atmosphere. The mixture was stirred at −78° C. for 15 min and then slowly warmed to −10° C. and stirred for 30 min under N₂ atmosphere. To the reaction mixture was added 6-bromo-7-methoxy-isochroman-4-one (500 mg, 1.94 mmol, 1 eq) in THF (5 mL) at −78° C. in dropwise under N2 atmosphere. The mixture was stirred at −78° C. for 1 h under N₂ atmosphere. After 1 h, diethyl oxalate was added dropwise to the mixture (454.77 mg, 3.11 mmol, 425.02 μL, 1.6 eq) in THF (2 mL) at −78° C. The reaction mixture was slowly brought to 0° C. and stirred for 1 h under N₂. The reaction mixture was cooled to −5° C. then acidified to pH=6 with 1 M HCl aqueous solution, There were precipitates formed in the reaction mixture. The mixture was filtered and the filter cake was collected and dried in vacuum to afford ethyl 2-(6-bromo-7-methoxy-4-oxo-isochroman-3-yl)-2-oxo-acetate (410 mg, crude) as a yellow solid. ¹H NMR (400 MHz, DMSO-d₆) δ=7.96 (s, 1H), 7.17 (s, 1H), 5.10 (s, 2H), 4.30-4.15 (m, 2H), 3.95 (s, 3H), 1.26 (t, J=7.2 Hz, 3H).

### Synthesis of ethyl 8-bromo-1-(3,5-dichlorophenyl)-7-methoxy-5H-isochromeno[4,3-c]pyrazole-3-carboxylate

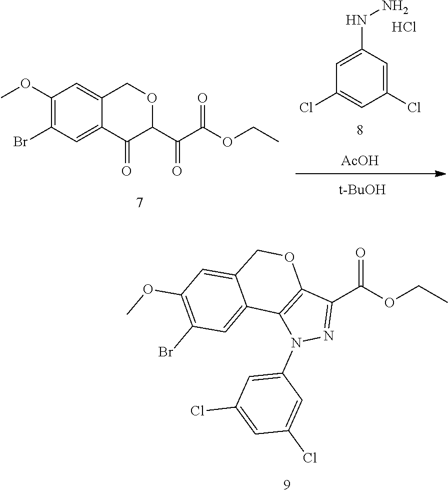

To a solution of ethyl 2-(6-bromo-7-methoxy-4-oxo-isochroman-3-yl)-2-oxo-acetate (410 mg, 1.15 mmol, 1 eq) in t-BuOH (5 mL) was added AcOH (344.68 mg, 5.74 mmol, 328.58 μL, 5 eq) and (3,5-dichlorophenyl)hydrazine hydrochloride (245.08 mg, 1.15 mmol, 1 eq) under N₂ atmosphere. The mixture was stirred at 110° C. for 16 hr. The reaction mixture was concentrated under reduced pressure to give a residue. The residue was triturated with EtOH (20 mL) at 25° C. for 30 min. It was filtered and the filter cake was collected and dried in vacuo. Compound ethyl 8-bromo-1-(3,5-dichlorophenyl)-7-methoxy-5H-isochromeno[4, 3-c]pyrazole-3-carboxylate (420 mg, 843.11 μmol, 73% yield) was obtained as an off-white solid. ¹H NMR (400 MHz, DMSO-d₆) δ=7.90 (t, J=2.0 Hz, 1H), 7.80 (d, J=2.0 Hz, 2H), 7.29 (s, 1H), 6.94 (s, 1H), 5.30 (s, 2H), 4.32 (q, J=7.2 Hz, 2H), 3.89 (s, 3H), 1.30 (t, J=7.2 Hz, 3H).

### Synthesis of 8-bromo-1-(3,5-dichlorophenyl)-7-methoxy-5H-isochromeno[4,3-c]pyrazole-3-carboxylic acid

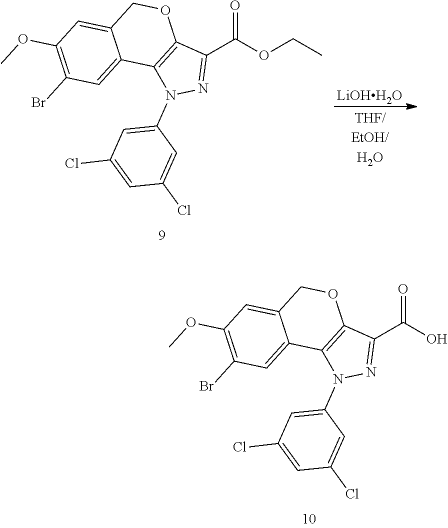

To a solution of ethyl 8-bromo-1-(3, 5-dichlorophenyl)-7-methoxy-5H-isochromeno[4, 3-c]pyrazole-3-carboxylate (420 mg, 843.11 μmol, 1 eq) in THF (5 mL) and EtOH (2 mL) was added a solution of LiOH·H₂O (70.76 mg, 1.69 mmol, 2 eq) in H₂O (5 mL). The mixture was stirred at 40° C. for 16 hr. The reaction mixture was concentrated under reduced pressure to give a residue. The reaction mixture was acidified to pH=6 with 1 M HCl aqueous solution, There were precipitates formed in the reaction mixture. The mixture was filtered and the filter cake was collected and dried in vacuum. Compound 8-bromo-1-(3,5-dichlorophenyl)-7-methoxy-5H-isochromeno[4,3-c]pyrazole-3-carboxylic acid (500 mg, crude) was obtained as a white solid. ¹H NMR (400 MHz, DMSO-d₆) δ=7.88 (t, J=2.0 Hz, 1H), 7.79 (d, J=2.0 Hz, 2H), 7.30 (s, 1H), 6.96 (s, 1H), 5.27 (s, 2H), 3.89 (s, 3H).

### Synthesis of [8-bromo-1-(3,5-dichlorophenyl)-7-methoxy-5H-isochromeno[4,3-c]pyrazol-3-yl]-(3,3-dimethylmorpholin-4-yl)methanone

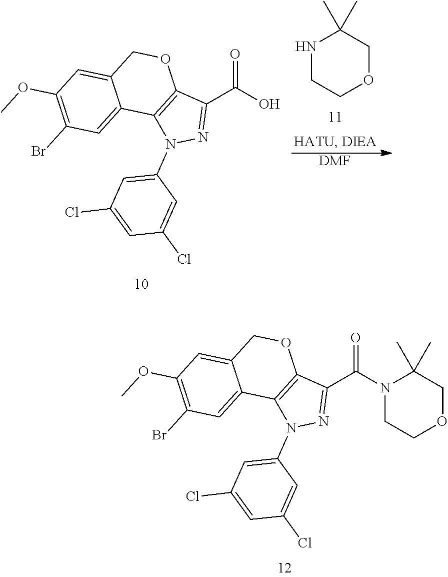

To a solution of 8-bromo-1-(3,5-dichlorophenyl)-7-methoxy-5H-isochromeno[4,3-c]pyrazole-3-carboxylic acid (250 mg, 531.80 μmol, 1 eq) in DMF (2 mL) was added HATU (202.21 mg, 531.80 μmol, 1 eq) and DIEA (206.19 mg, 1.60 mmol, 277.89 μL, 3 eq). The mixture stirred at 25° C. for 30 min. Then to the mixture was added a mixture of 3,3-dimethylmorpholine (73.50 mg, 638.16 μmol, 1.2 eq) in DMF (1 mL). The mixture stirred at 25-40° C. for 18 hr. The reaction mixture was diluted with H₂O (10 mL), extracted with EtOAc (20 mL*3). The combined organic phases were washed with brine (20 mL), dried over anhydrous Na₂SO₄, It was filtered and the filtrate was concentrated under reduced pressure to give a residue. The residue was purified by flash silica gel chromatography (ISCO®; 4 g SepaFlash® Silica Flash Column, Eluent of 0˜40% Ethyl acetate/Petroleum ether gradient @40 mL/min). Compound [8-bromo-1-(3,5-dichlorophenyl)-7-methoxy-5H-isochromeno[4,3-c]pyrazol-3-yl]-(3,3dimethylmorpholin-4-yl)methanone (180 mg, 317.32 μmol, 60% yield) was obtained as a yellow solid. ¹H NMR (400 MHz, CHLOROFORM-d) δ=7.52 (d, J=1.6 Hz, 2H), 7.46 (t, J=1.6 Hz, 1H), 7.17 (s, 1H), 6.82 (s, 1H), 5.22 (s, 2H), 3.94 (s, 3H), 3.86-3.80 (m, 2H), 3.73-3.67 (m, 2H), 3.50-3.47 (m, 2H), 1.55 (s, 6H).

### Synthesis of 5-[1-(3,5-dichlorophenyl)-3-(3,3-dimethylmorpholine-4-carbonyl)-7-methoxy-5H-isochromeno[4,3-c]pyrazol-8-yl]pyridine-3-carbonitrile

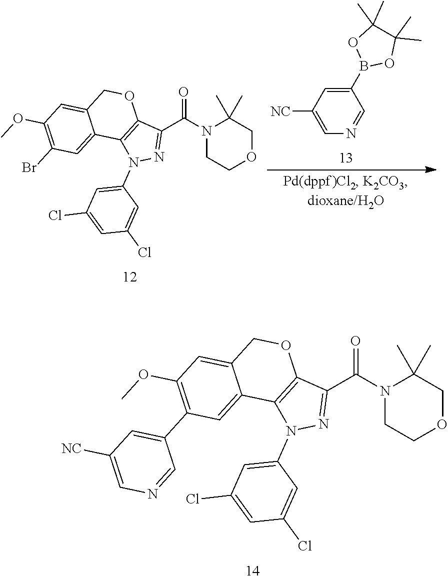 [SMILES: COc1cc2c(cc1Br)-c1c(c(C(=O)N3CCOCC3(C)C)nn1-c1cc(Cl)cc(Cl)c1)OC2 ; CC1(C)OB(c2cncc(C#N)c2)OC1(C)C ; COc1cc2c(cc1-c1cncc(C#N)c1)-c1c(c(C(=O)N3CCOCC3(C)C)nn1-c1cc(Cl)cc(Cl)c1)OC2]

To a mixture of [8-bromo-1-(3,5-dichlorophenyl)-7-methoxy-5H-isochromeno[4,3-c]pyrazol-3-yl]-(3,3-dimethylmorpholin-4-yl)methanone (108 mg, 190.39 μmol, 1 eq) and 5-(4,4,5,5-tetramethyl-1,3,2-dioxaborolan-2-yl) pyridine-3-carbonitrile (45.99 mg, 199.91 μmol, 1.05 eq) in dioxane (3 mL) and H₂O (1.5 mL) was added K₂CO₃ (52.63 mg, 380.78 μmol, 2 eq), Pd(dppf)Cl₂ (13.93 mg, 19.04 μmol, 0.1 eq), the mixture was stirred at 60° C. for 16 h under N₂. The reaction mixture was diluted with ice water (10 mL). The aqueous Layer was extracted with ethyl acetate (10 mL*3). The combined organic layers were dried with anhydrous Na₂SO₄, The mixture was filtered and the filtrate was concentrated in vacuum to give a residue. The residue was purified by flash silica gel chromatography (ISCO®; 4 g SepaFlash® Silica Flash Column, Eluent of 0˜42% Ethyl acetate/Petroleum ether gradient @40 mL/min). The desired compound, 5-[1-(3,5-dichlorophenyl)-3-(3,3-dimethylmorpholine-4-carbonyl-7-methoxy-5H-isochromeno[4,3-c]pyrazol-8-yl]pyridine-3-carbonitrile (75 mg, 127.02 μmol, 67% yield), was obtained as a yellow solid.

### Synthesis of 5-[1-(3,5-dichlorophenyl)-3-(3,3-dimethylmorpholine-4-carbonyl)-7-methoxy-5H-isochromeno[4,3-c]pyrazol-8-yl]pyridine-3-carboxamide

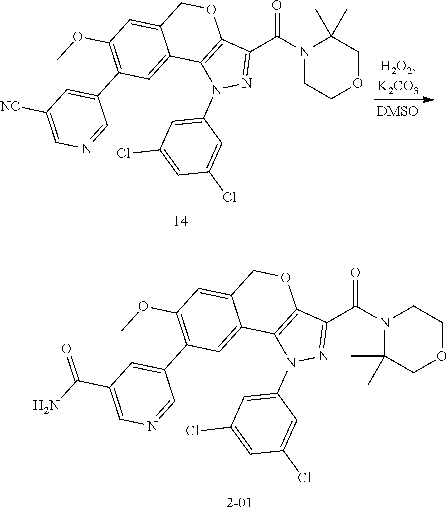 [SMILES: COc1cc2c(cc1-c1cncc(C#N)c1)-c1c(c(C(=O)N3CCOCC3(C)C)nn1-c1cc(Cl)cc(Cl)c1)OC2 ; COc1cc2c(cc1-c1cncc(C(N)=O)c1)-c1c(c(C(=O)N3CCOCC3(C)C)nn1-c1cc(Cl)cc(Cl)c1)OC2]

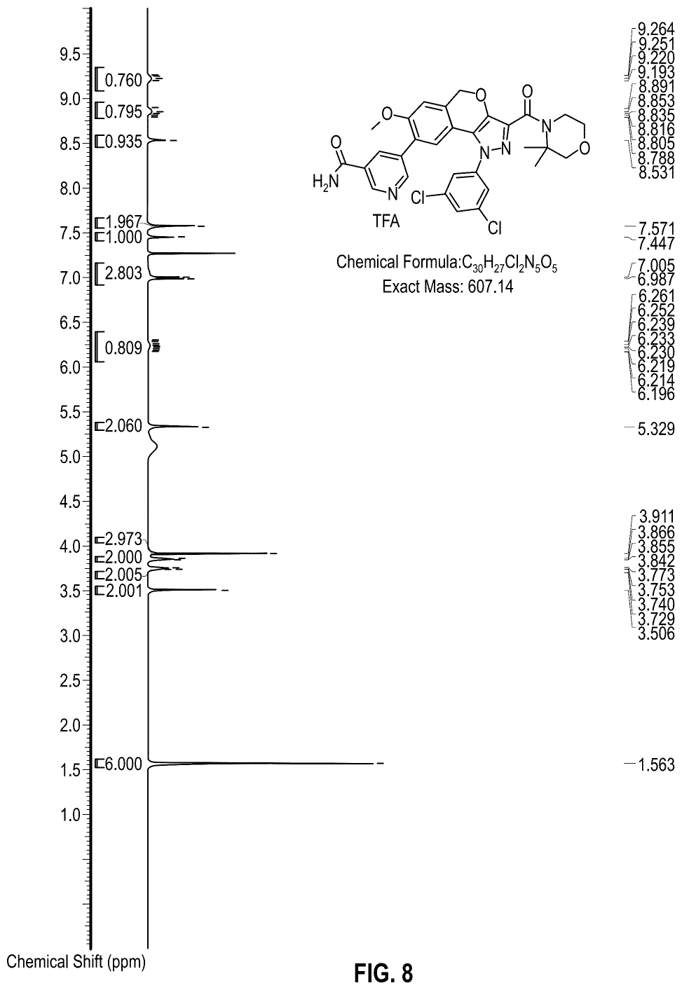

To a solution of 5-[1-(3,5-dichlorophenyl)-3-(3,3-dimethylmorpholine-4-carbonyl)-7-methoxy-5H-isochromeno[4,3-c]pyrazol-8-yl]pyridine-3-carbonitrile (75 mg, 127.02 μmol, 1 eq) in DMSO (1 mL) was added K₂CO₃ (3 M, 84.68 μL, 2 eq) and H₂O₂ (120 mg, 1.06 mmol, 101.69 μL, 30% purity, 8.33 eq). The mixture was stirred at 25° C. for 1 hr. The reaction mixture was quenched with sat.Na₂SO₃ (10 mL). The aqueous layer was extracted with ethyl acetate (10 mL*3). The combined organic layers were washed with brine and dried with anhydrous Na2SO4, It was filtered and the filtrate was concentrated in vacuum to give a residue. The residue was purified by prep-HPLC (column: Phenomenex Luna C18 150*25 mm*10 um; mobile phase: [water (TFA) -ACN]; gradient: 38%-68% B over min), followed by lyophilization. Compound 5-[1-(3,5-dichlorophenyl)-3-(3,3-dimethylmorpholine-4-carbonyl)-7-methoxy-5H-isochromeno[4,3-c]pyrazol-8-yl]pyridine-3-carboxamide (22.74 mg, 31.47 μmol, 25% yield, 100% purity, TFA) was obtained as a yellow solid. LCMS (ESI): m/z [M+H] calcd for C₃₀H₈₈C₁₂N₅O₅: 608.14; found: 608.3. ¹H NMR (400 MHz, CHLOROFORM-d) δ=9.34-9.09 (m, 1H), 8.95-8.77 (m, 1H), 8.53 (s, 1H), 7.57 (s, 2H), 7.45 (s, 1H), 7.00 (d, J=7.2 Hz, 3H), 6.39-6.06 (m, 1H), 5.33 (s, 2H), 3.91 (s, 3H), 3.88-3.82 (m, 2H), 3.78-3.70 (m, 2H), 3.51 (s, 2H), 1.56 (s, 6H). FIG. 8 shows the nuclear magnetic resonance of Compound 2-01.

### Synthesis of [1-(3,5-dichlorophenyl)-7-methoxy-8-(1-methylpyrazol-3-yl)-5H-isochromeno[4,3-c]pyrazol-3-yl]-(3,3-dimethylmorpholin-4-yl)methanone

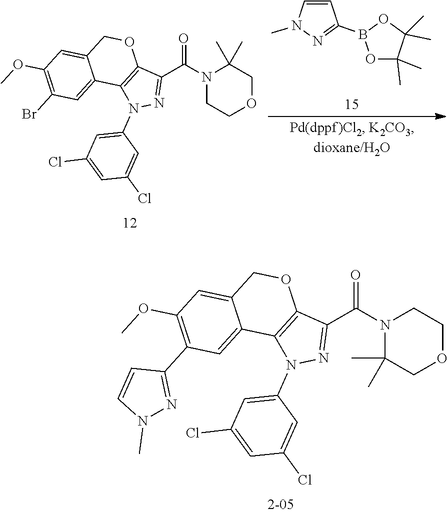

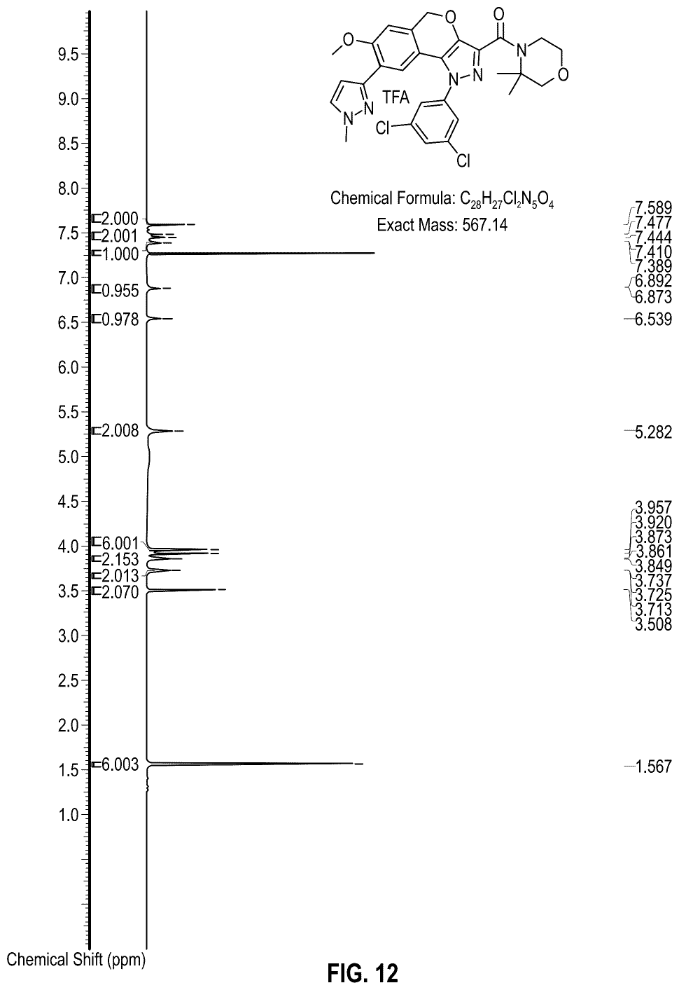

To a mixture of [8-bromo-1-(3,5-dichlorophenyl)-7-methoxy-5H-isochromeno[4,3-c]pyrazol-3-yl]-(3,3-dimethylmorpholin-4-yl)methanone (72 mg, 126.93 μmol, 1 eq) and 1-methyl-3-(4,4,5,5-tetramethyl-1,3,2-dioxaborolan-2-yl)pyrazole (31.69 mg, 152.31 μmol, 1.2 eq) in dioxane (2 mL) and H₂O (1 mL) was added K₂CO₃ (35.08 mg, 253.85 μmol, 2 eq), Pd(dppf)Cl₂ (9.29 mg, 12.69 μmol, 0.1 eq), the mixture was stirred at 80° C. for 16 h under N₂. The reaction mixture was filtered and concentrated under reduced pressure to give a residue. The residue was purified by prep-HPLC (column: Phenomenex Luna C18 150*25 mm*10 um; mobile phase: [water (TFA) -ACN]; gradient: 55%-85% B over min), followed by lyophilization. Compound [1-(3,5-dichlorophenyl)-7-methoxy-8-(1-methylpyrazol-3-yl)-5H-isochromeno[4,3-c]pyrazol-3-yl]-(3,3-dimethylmorpholin-4-yl)methanone (25.06 mg, 36.72 μmol, 29% yield, 100% purity, TFA) was obtained as a gray solid. LCMS (ESI): m/z [M+H] calcd for C₂₈H₂₈C₁₂N₅O₄: 568.14; found: 568.3. ¹H NMR (400 MHz, CHLOROFORM-d) δ=7.59 (s, 2H), 7.46 (br d, J=13.2 Hz, 2H), 7.41-7.36 (m, 1H), 6.91-6.82 (m, 1H), 6.54 (s, 1H), 5.28 (br s, 2H), 3.94 (br d, J=14.8 Hz, 6H), 3.88-3.83 (m, 2H), 3.73 (br t, J=4.8 Hz, 2H), 3.51 (s, 2H), 1.57 (s, 6H). FIG. 12 shows the nuclear magnetic resonance of Compound 2-05.

### Synthesis of Compound 2-02

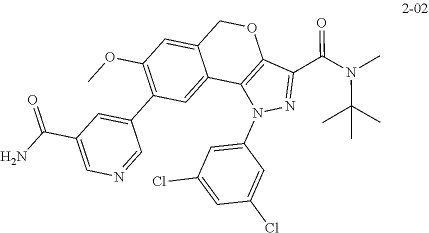 [SMILES: COc1cc2c(cc1-c1cncc(C(N)=O)c1)-c1c(c(C(=O)N(C)C(C)(C)C)nn1-c1cc(Cl)cc(Cl)c1)OC2]

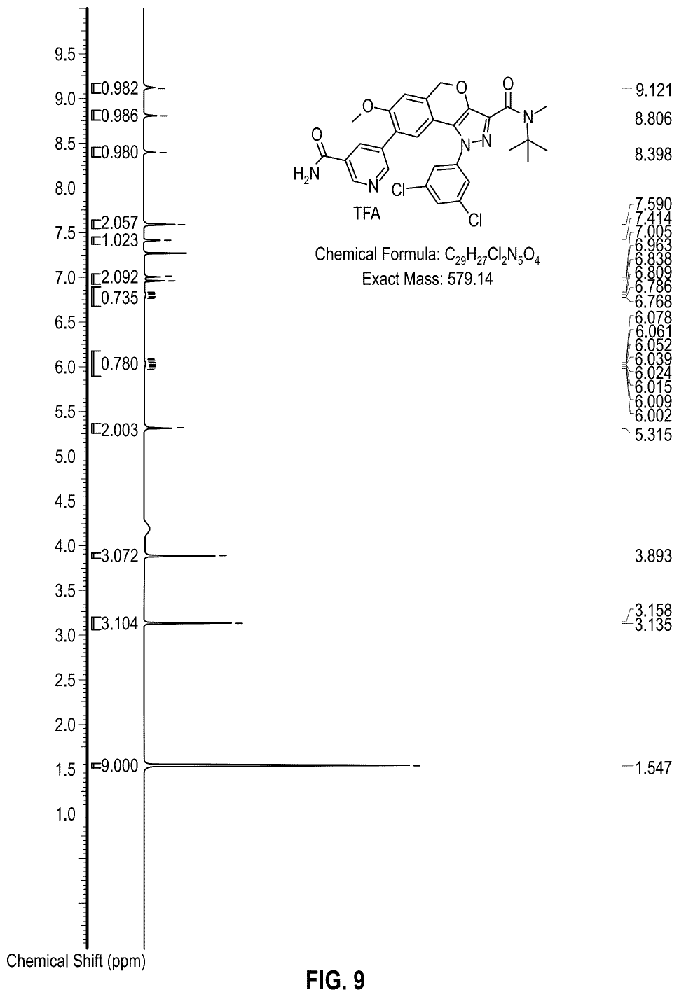

Compound 2-02 was synthesized in a procedure similar to example 1. LCMS (ESI): m/z [M+H] calcd for C₂₉H₂₃Cl₂N₅O₄: 580.14; found: 580.3. ¹H NMR (400 MHz, CHLOROFORM-d) δ=9.12 (s, 1H), 8.81 (s, 1H), 8.40 (s, 1H), 7.59 (s, 2H), 7.41 (s, 1H), 6.98 (d, J=16.8 Hz, 2H), 6.90-6.68 (m, 1H), 6.17-5.91 (m, 1H), 5.31 (s, 2H), 3.89 (s, 3H), 3.14 (s, 3H), 1.55 (s, 9H). FIG. 9 shows the nuclear magnetic resonance of Compound 2-02.

### Synthesis of Compound 2-03

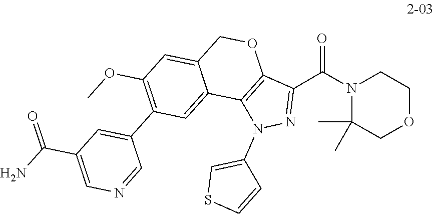 [SMILES: COc1cc2c(cc1-c1cncc(C(N)=O)c1)-c1c(c(C(=O)N3CCOCC3(C)C)nn1-c1ccsc1)OC2]

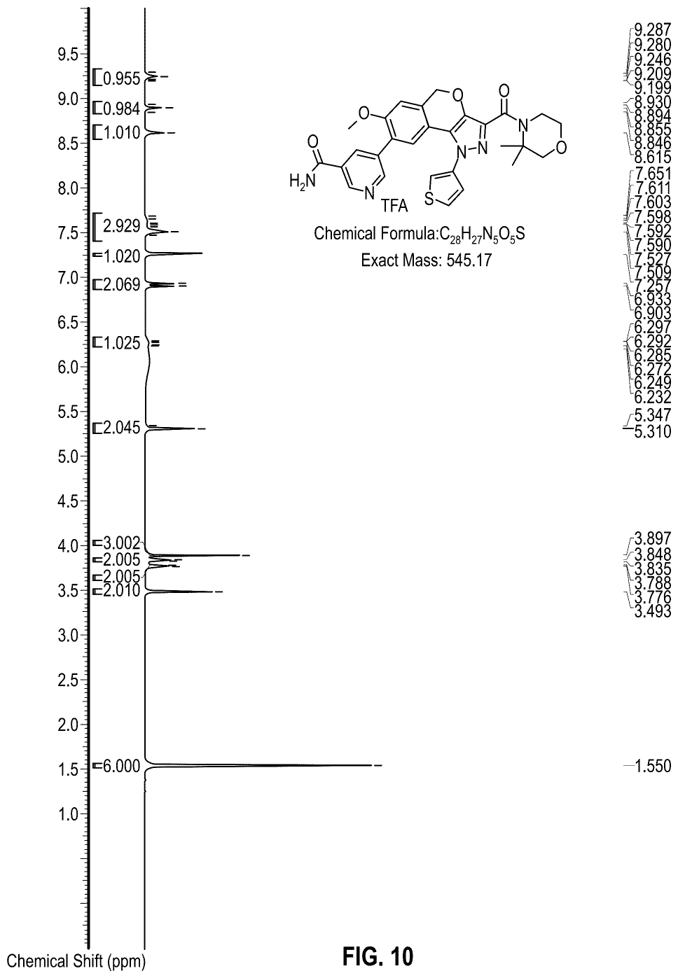

Compound 2-03 was synthesized in a procedure similar to example 1. LCMS (ESI): m/z [M+H] calcd for C₂₈H₂₈N₅O₅S 546.17; found: 546.3. ¹H NMR (400 MHz, CHLOROFORM-d) δ=9.33-9.15 (m, 1H), 8.89 (s, 1H), 8.62 (s, 1H), 7.72-7.41 (m, 3H), 7.26 (br s, 1H), 6.92 (d, J=12.0 Hz, 2H), 6.33-6.22 (m, 1H), 5.31 (s, 2H), 3.90 (s, 3H), 3.84 (br d, J=4.8 Hz, 2H), 3.78 (br d, J=4.8 Hz, 2H), 3.49 (s, 2H), 1.55 (s, 6H). FIG. 10 shows the nuclear magnetic resonance of Compound 2-03.

### Synthesis of Compound 2-04

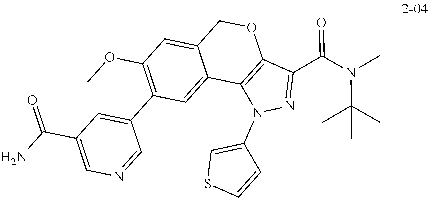 [SMILES: COc1cc2c(cc1-c1cncc(C(N)=O)c1)-c1c(c(C(=O)N(C)C(C)(C)C)nn1-c1ccsc1)OC2]

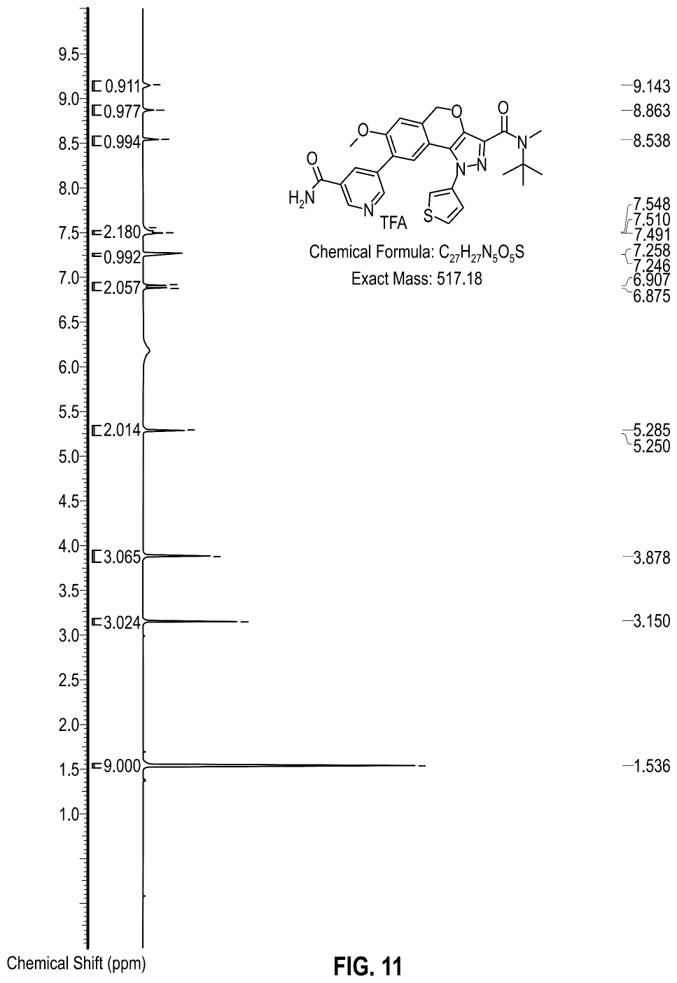

Compound 2-04 was synthesized in a procedure similar to example 1. LCMS (ESI): m/z [M+H] calcd for C₂₇H₂₈N₅O₄S: 518.18; found: 518.3. ¹H NMR (400 MHz, CHLOROFORM-d) δ=9.14 (br s, 1H), 8.86 (br s, 1H), 8.54 (s, 1H), 7.53-7.47 (m, 2H), 7.25 (br d, J=4.8 Hz, 1H), 6.89 (d, J=12.8 Hz, 2H), 5.29 (s, 2H), 3.88 (s, 3H), 3.15 (s, 3H), 1.54 (s, 9H). FIG. 11 shows the nuclear magnetic resonance of Compound 2-04.

### Synthesis of Compound 2-06

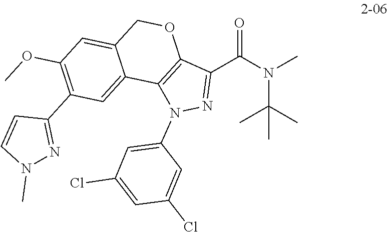 [SMILES: COc1cc2c(cc1-c1ccn(C)n1)-c1c(c(C(=O)N(C)C(C)(C)C)nn1-c1cc(Cl)cc(Cl)c1)OC2]

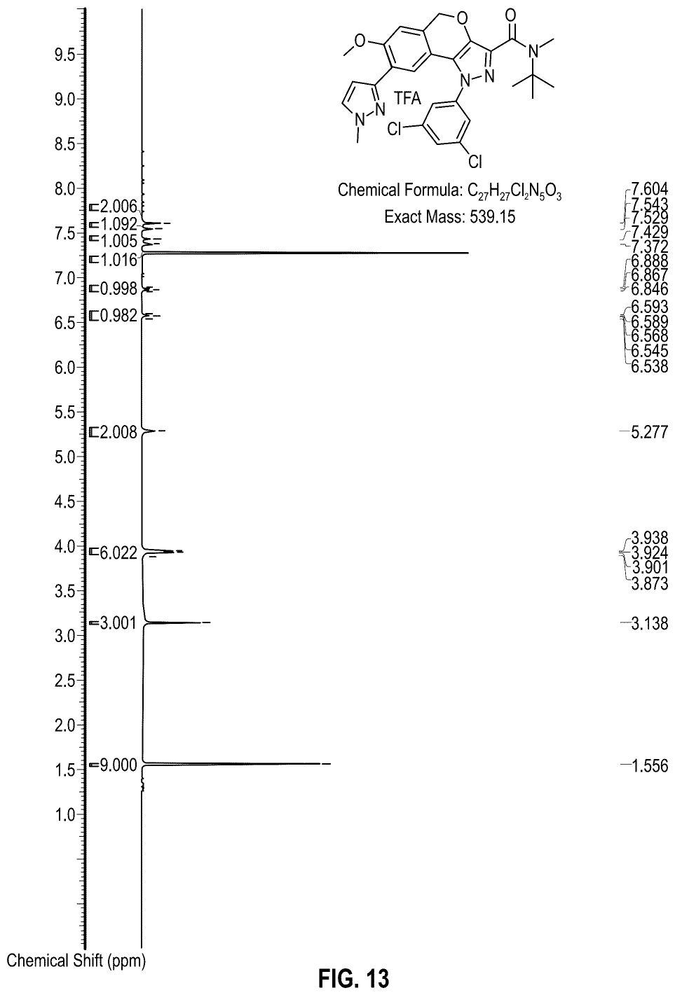

Compound 2-06 was synthesized in a procedure similar to example 1. LCMS (ESI): m/z [M+H] calcd for C₂₇H₂₈C₁₂N₅O₃: 540.15; found: 540.3. ¹H NMR (400 MHz, CHLOROFORM-d) δ=7.60 (s, 2H), 7.56-7.51 (m, 1H), 7.43 (s, 1H), 7.37 (br s, 1H), 6.91-6.83 (m, 1H), 6.61-6.52 (m, 1H), 5.28 (s, 2H), 3.98-3.90 (m, 6H), 3.14 (s, 3H), 1.56 (s, 9H). FIG. 13 shows the nuclear magnetic resonance of Compound 2-06.

### Synthesis of Compound 2-07

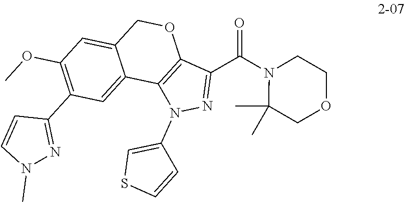 [SMILES: COc1cc2c(cc1-c1ccn(C)n1)-c1c(c(C(=O)N3CCOCC3(C)C)nn1-c1ccsc1)OC2]

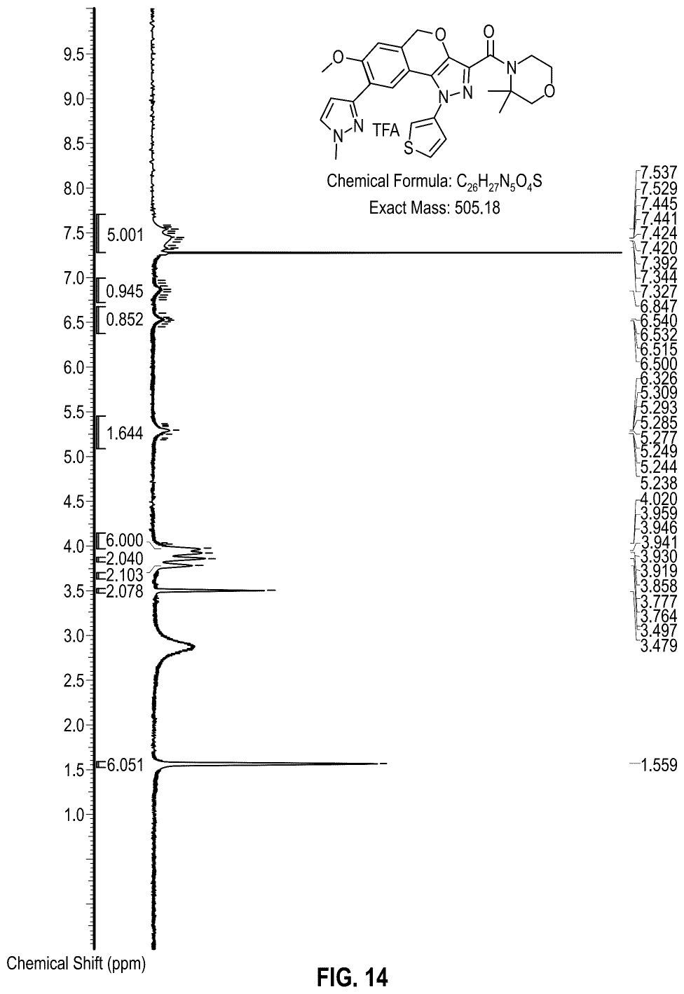

Compound 2-07 was synthesized in a procedure similar to example 1. LCMS (ESI): m/z [M+H] calcd for C₂₆H₂₈N₅O₄S: 506.18; found: 506.4. ¹H NMR (400 MHz, CHLOROFORM-d) δ=7.69-7.27 (m, 5H), 6.98-6.72 (m, 1H), 6.67-6.37 (m, 1H), 5.45-5.08 (m, 2H), 4.04-3.87 (m, 6H), 3.86 (br s, 2H), 3.77 (br d, J=5.2 Hz, 2H), 3.52-3.47 (m, 2H), 1.56 (s, 6H). FIG. 14 shows the nuclear magnetic resonance of Compound 2-07.

### Synthesis of Compound 2-08

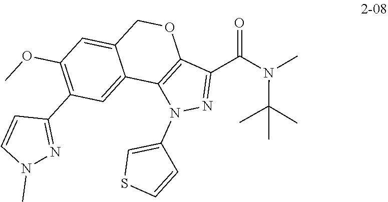 [SMILES: COc1cc2c(cc1-c1ccn(C)n1)-c1c(c(C(=O)N(C)C(C)(C)C)nn1-c1ccsc1)OC2]

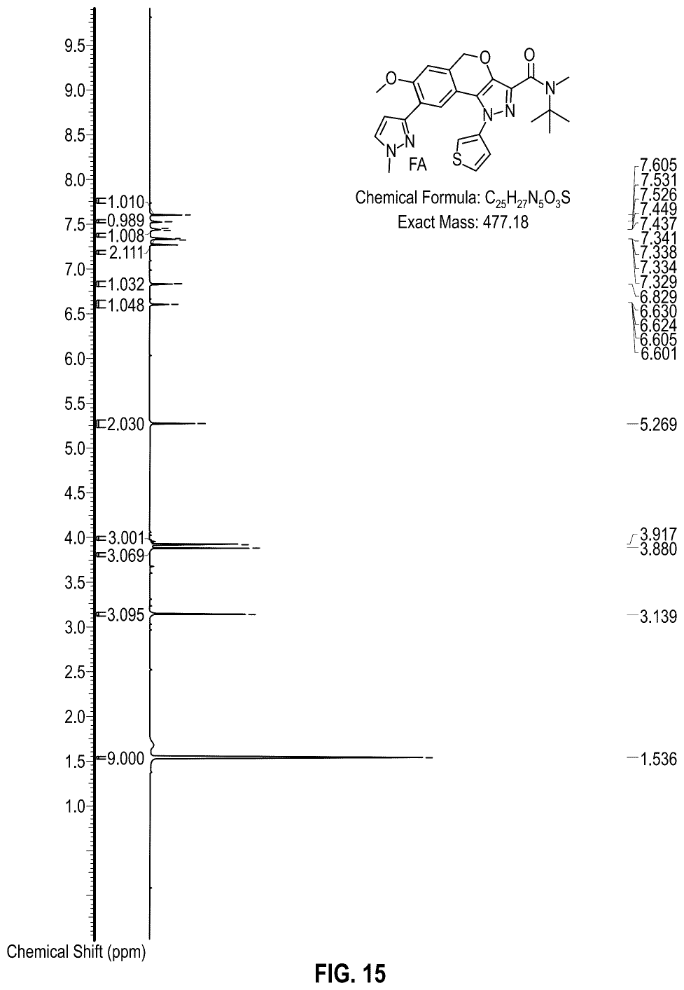

Compound 2-08 was synthesized in a procedure similar to example 1. LCMS (ESI): m/z [M+H] calcd for C₂₅H₂₈N₅O₃S: 578.18; found: 578.3. ¹H NMR (400 MHz, CHLOROFORM-d) δ=7.60 (s, 1H), 7.53 (d, J=2.0 Hz, 1H), 7.44 (dd, J=3.2, 5.2 Hz, 1H), 7.36-7.31 (m, 2H), 6.83 (s, 1H), 6.60 (d, J=2.0 Hz, 1H), 5.27 (s, 2H), 3.92 (s, 3H), 3.88 (s, 3H), 3.14 (s, 3H), 1.54 (s, 9H). FIG. 15 shows the nuclear magnetic resonance of Compound 2-08.

### Example 2

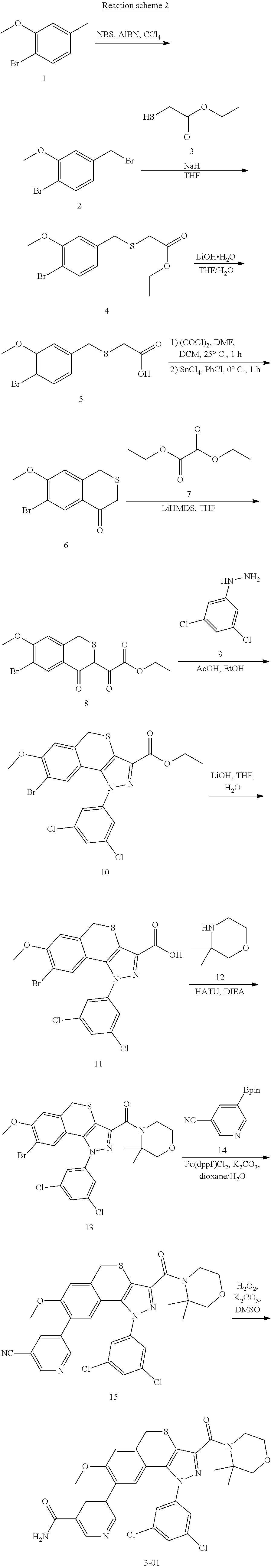

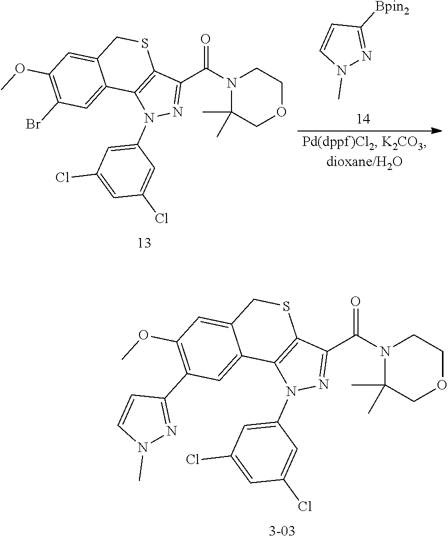

### Synthesis of 1-bromo-4-(bromomethyl)-2-methoxy-benzene

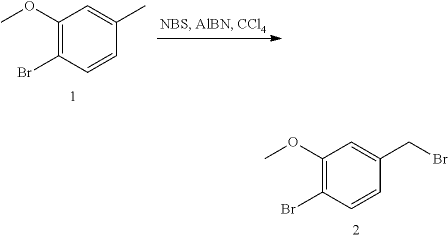

A mixture of 1-bromo-2-methoxy-4-methyl-benzene (25 g, 124.34 mmol, 1 eq), AIBN (2.04 g, 12.43 mmol, 0.1 eq) and NBS (24.34 g, 136.78 mmol, 1.1 eq) in CCl₄ (300 mL) was degassed and purged with N₂ for 3 times, and then the mixture was stirred at 80° C. for 16 hr under N₂ atmosphere. The mixture was concentrated to the get the residue. The residue was purified by flash silica gel chromatography (ISCO®; 220 g SepaFlash® Silica Flash Column, Eluent of 0˜7% Ethyl acetate/Petroleum ether gradient @80 m/min). Compound 1-bromo-4-(bromomethyl)-2-methoxy-benzene (34 g, 121.45 mmol, 98% yield) was obtained as a colorless oil. ¹H NMR (400 MHz, CHLOROFORM-d) δ=7.50 (d, J=8.0 Hz, 1H), 6.93 (d, J=1.9 Hz, 1H), 6.87 (dd, J=1.8, 8.1 Hz, 1H), 4.45 (s, 2H), 3.94-3.90 (m, 3H).

### Synthesis of ethyl 2-[(4-bromo-3-methoxy-phenyl)methylsulfanyl]acetate

To a solution of ethyl 2-sulfanylacetate (3.85 g, 32.04 mmol, 3.51 mL, 1.28 eq) in THF (70 mL) was added NaH (1.50 g, 37.51 mmol, 60% purity, 1.5 eq) at 0° C. in dropwise. After addition, the mixture was stirred at 0° C. for 0.5 hr, and then 1-bromo-4-(bromomethyl)-2-methoxy-benzene (7 g, 25.00 mmol, 1 eq) in THF (30 mL) was added at 0° C. in dropwise. The resulting mixture was stirred at 25° C. for 2 hr. The reaction was quenched with sat. NH₄C₁ (50 mL) at 0° C. The mixture was poured into ice-water (100 mL) and stirred for 5 min. The aqueous phase was extracted with ethyl acetate (100 mL*3). The combined organic phases were washed with brine (200 mL), dried over anhydrous Na₂SO₄, It was filtered and the filtrate was concentrated in vacuum to get the residue. The residue was purified by flash silica gel chromatography (ISCO®; 80 g SepaFlash® Silica Flash Column, Eluent of 0˜25% Ethyl acetate/Petroleum ether gradient @60 mL/min). Compound ethyl 2-[(4-bromo-3-methoxy-phenyl)methylsulfanyl]acetate (5.9 g, 18.48 mmol, 74% yield) was obtained as a colorless oil ¹H NMR (400 MHz, CHLOROFORM-d) δ=7.46 (d, J=8.0 Hz, 1H), 6.92 (d, J=1.4 Hz, 1H), 6.81 (dd, J=1.5, 8.0 Hz, 1H), 4.18 (q, J=7.1 Hz, 2H), 3.90 (s, 3H), 3.79 (s, 2H), 3.06 (s, 2H), 1.29 (t, J=7.1 Hz, 3H).

### Synthesis of 2-[(4-bromo-3-methoxy-phenyl)methylsulfanyl]acetic acid

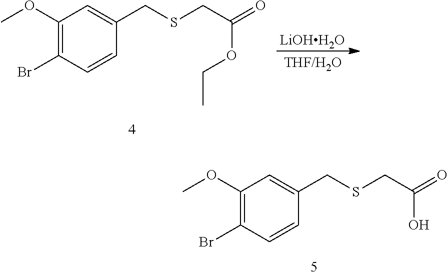

A mixture of ethyl 2-[(4-bromo-3-methoxy-phenyl)methylsulfanyl]acetate (14.5 g, 45.42 mmol, 1 eq) and LiOH. H₂O (7.62 g, 181.70 mmol, 30 mL, 4 eq) in EtOH (150 mL) was stirred at 25° C. for 2 hr under N₂ atmosphere. The mixture was poured into ice-water (200 mL), the reaction mixture was acidified to pH=6 with 1 M HCl aqueous solution, the aqueous phase was extracted with ethyl acetate (150 mL*3). The combined organic phases were washed with brine (500 mL), dried with anhydrous Na₂SO₄, It was filtered and the filtrate was concentrated to get the product. The crude product 2-[(4-bromo-3-methoxy-phenyl)methylsulfanyl]acetic acid (12 g, crude) was obtained as a colorless oil used directly without further purification. ¹H NMR (400 MHz, METHANOL-d₄) δ=7.44 (d, J=8.0 Hz, 1H), 7.02 (s, 1H), 6.83 (br d, J=7.8 Hz, 1H), 3.87 (s, 3H), 3.81 (s, 2H), 3.12 (s, 2H).

### Synthesis of 6-bromo-7-methoxy-isothiochroman-4-one

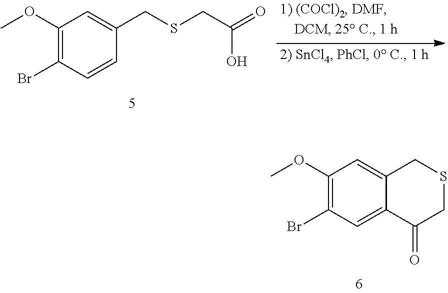

To a solution of 2-[(4-bromo-3-methoxy-phenyl) methylsulfanyl]acetic acid (12 g, 41.21 mmol, 1 eq) in DCM (100 mL) was added SOCl₂ (151.20 g, 1.27 mol, 92.31 mL, 30.84 eq). The mixture was stirred at 50° C. for 2 hr. The mixture was concentrated to get the crude product. The crude product 2-[(4-bromo-3-methoxy-phenyl)methylsulfanyl]acetyl chloride (13 g, crude) was obtained as a yellow oil used directly without further purification.

To a solution of 2-[(4-bromo-3-methoxy-phenyl)methylsulfanyl]acetyl chloride (13 g, 41.99 mmol, 1 eq) in chlorobenzene (100 mL) was added SnCl₄ (1 M, 83.98 mL, 2 eq) at 0° C. The mixture was stirred at 0° C. for 1 hr. The reaction was quenched with sat. NaHCO₃ (200 mL) and then filtered through a celite pad. The filtrate was poured into ice-water (200 mL) and stirred for 5 min. The aqueous phase was extracted with ethyl acetate (200 mL*3). The combined organic phases were washed with brine (500 mL), dried with anhydrous Na₂SO₄, filtered and the filtrate was concentrated to get the residue. The residue was purified by flash silica gel chromatography (ISCO®; 220 g SepaFlash® Silica Flash Column, Eluent of 0˜30% Ethyl acetate/Petroleum ether gradient @80 mL/min). Compound 6-bromo-7-methoxy-isothiochroman-4-one (8 g, 29.29 mmol, 70% yield) was obtained as a red solid. ¹H NMR (400 MHz, CHLOROFORM-d) δ=8.28 (s, 1H), 6.66 (s, 1H), 3.96 (s, 3H), 3.86 (s, 2H), 3.51 (s, 2H).

### Synthesis of ethyl 2-(6-bromo-7-methoxy-4-oxo-isothiochroman-3-yl)-2-oxo-acetate

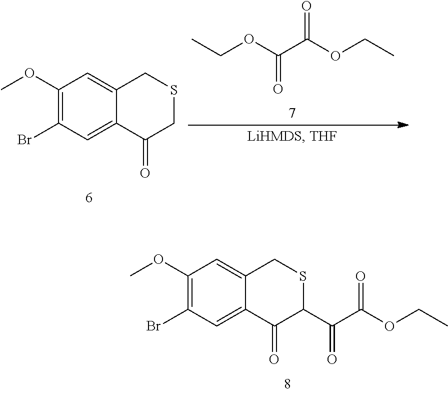

To a solution of 6-bromo-7-methoxy-isothiochroman-4-one (1.1 g, 4.03 mmol, 1 eq) in THF (15 mL) was added LiHMDS (1 M, 5.24 mL, 1.3 eq) at −70° C. in dropwise for 10 mi. After addition, the mixture was stirred at −70° C. for 0.5 hr, and then diethyl oxalate (882.80 mg, 6.04 mmol, 825.04 μL, 1.5 eq) in THF (5 mL) was added in dropwise at −70° C. The resulting mixture was stirred at 0° C. for 1 hr. The mixture was poured into ice-water (20 mL), the reaction mixture was acidified to pH=6 with 1 M HCl aqueous solution, There were precipitates formed in the reaction mixture. The mixture was filtered and the filter cake was collected and dried in vacuum. Compound ethyl 2-(6-bromo-7-methoxy-4-oxo-isothiochroman-3-yl)-2-oxo-acetate (1.2 g, 3.22 mmol, 80% yield) was obtained as a yellow solid. ¹H NMR (400 MHz, DMSO-d₆) δ=7.93 (s, 1H), 7.15 (s, 1H), 4.24 (q, J=7.0 Hz, 2H), 3.94 (s, 3H), 3.90 (s, 2H), 1.28 (t, J=7.1 Hz, 3H).

### Synthesis of 8-bromo-1-(3,5-dichlorophenyl)-7-methoxy-5H-isothiochromeno[4,3-c]pyrazole-3-carboxylate

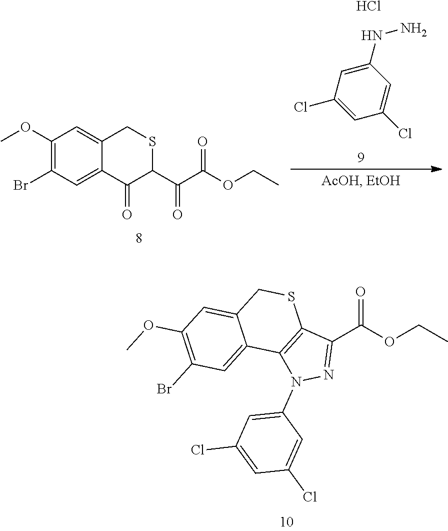

A mixture of ethyl 2-(6-bromo-7-methoxy-4-oxo-isothiochroman-3-yl)-2-oxo-acetate (130 mg, 348.32 μmol, 1 eq), (3,5-dichlorophenyl)hydrazine;hydrochloride (74.36 mg, 348.32 μmol, 1 eq) and AcOH (209.80 mg, 3.49 mmol, 0.2 mL, 10.03 eq) in EtOH (2 mL) was stirred at 80° C. for 5 hr under N₂ atmosphere. The mixture was filtered and the filter cake was collected and dried in vacuum to get the crude product. The crude product ethyl 8-bromo-1-(3,5-dichlorophenyl)-7-methoxy-5H-isothiochromeno[4,3-c]pyrazole-3-carboxylate (0.17 g, crude) was obtained as a black solid used directly without further purification. LCMS (ESI): m/z [M+H] calcd for C₂₀H₁₆BrCl₂N₂O₃S: 512.94; found: 512.8. ¹H NMR (400 MHz, DMSO-d₆) δ=7.91-7.87 (m, 1H), 7.77 (d, J=1.6 Hz, 2H), 7.33 (s, 1H), 6.88 (s, 1H), 4.36-4.32 (m, 2H), 4.08 (s, 2H), 3.91-3.90 (m, 3H), 1.34-1.30 (m, 3H).

### Synthesis of 8-bromo-1-(3,5-dichlorophenyl)-7-methoxy-5H-isothiochromeno[4,3-c]pyrazole-3-carboxylic acid

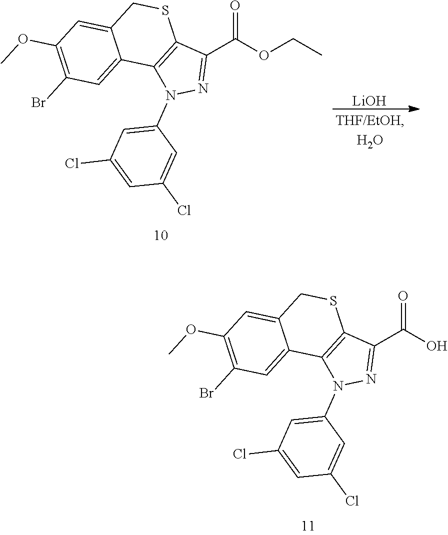

A mixture of ethyl 8-bromo-1-(3,5-dichlorophenyl)-7-methoxy-5H-isothiochromeno[4,3-c]pyrazole-3-carboxylate (1.4 g, 2.72 mmol, 1 eq) and LiOH. H2O (571.25 mg, 13.61 mmol, 6 mL, 5 eq) in THF (10 mL), EtOH (10 mL) and H2O (2 mL) was stirred at 25° C. for 16 hr under N2 atmosphere. The residue was poured into ice-water (20 mL) and the reaction mixture was acidified to pH=6 with 1 M HCl aqueous solution. The aqueous phase was extracted with ethyl acetate (20 mL*3). The combined organic phases were washed with brine (50 mL), dried with anhydrous Na2SO4, It was filtered and the filtrate was concentrated in vacuum to get the crude product. The crude product 8-bromo-1-(3,5-dichlorophenyl)-7-methoxy-5H-isothiochromeno[4,3-c]pyrazole-3-carboxylic acid (1.2 g, crude) was obtained as a red solid used directly without further purification. LCMS (ESI): m/z [M+H] calcd for C₁₈H₁₂BrCl₂N₂O₃S: 484.91; found: 484.7. ¹H NMR (400 MHz, METHANOL-d₄) δ=7.68-7.64 (m, 3H), 7.16 (s, 1H), 6.96 (s, 1H), 4.01 (s, 2H), 3.94 (s, 3H).

### Synthesis of (8-bromo-1-(3,5-dichlorophenyl)-7-methoxy-1,5-dihydroisothiochromeno[4,3-c]pyrazol-3-yl)(3,3-dimethylmorpholino)methanone

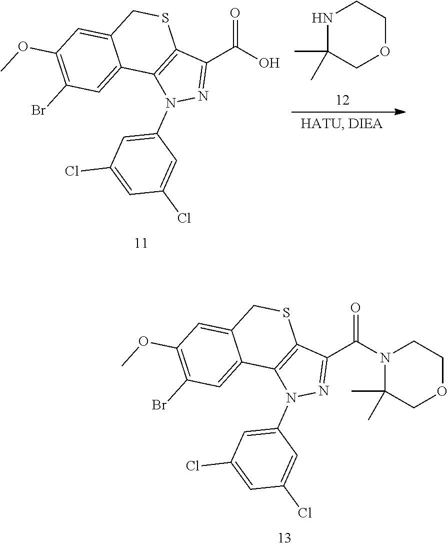

To a solution of 8-bromo-1-(3,5-dichlorophenyl)-7-methoxy-5H-isothiochromeno[4,3-c]pyrazole-3-carboxylic acid (0.6 g, 1.23 mmol, 1 eq) in THF (10 mL) was added HATU (703.89 mg, 1.85 mmol, 1.5 eq) and DIEA (478.51 mg, 3.70 mmol, 644.90 μL, 3 eq) at 25° C. After addition, the mixture was stirred at this temperature for 0.5 hr, and then 3,3-dimethylmorpholine (156.35 mg, 1.36 mmol, 1.1 eq) in THF (5 mL) was added at 25° C. The resulting mixture was stirred at 25° C. for 16 hr. The mixture was poured into ice-water (30 mL) and stirred for 5 min. The aqueous phase was extracted with ethyl acetate (20 mL*3). The combined organic phases were washed with brine (50 mL), dried with anhydrous Na₂SO₄, It was filtered and the filtrate was concentrated in vacuum to get the residue. The residue was purified by flash silica gel chromatography (ISCO®; 10 g SepaFlash® Silica Flash Column, Eluent of 0˜20% Ethyl acetate/Petroleum ethergradient @40 mL/min). Compound (8-bromo-1-(3,5-dichlorophenyl)-7-methoxy-1,5-dihydroisothiochromeno[4,3-c]pyrazol-3-yl)(3,3-dimethylmorpholino)methanone (0.6 g, 1.03 mmol, 83% yield) was obtained as a yellow oil. LCMS (ESI): m/z [M+H] calcd for C₂₄H₂₃BrCl₂N₃O₃S: 581.99; found: 582.0.

### Synthesis of 5-(1-(3,5-dichlorophenyl)-3-(3,3-dimethylmorpholine-4-carbonyl)-7-methoxy-1,5-dihydroisothiochromeno[4,3-c]pyrazol-8-yl)nicotinonitrile

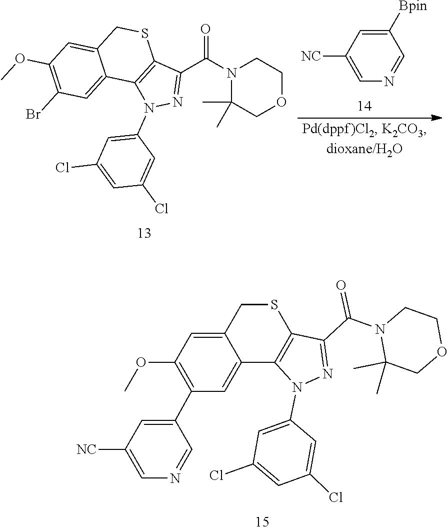

To a mixture of (8-bromo-1-(3,5-dichlorophenyl)-7-methoxy-1,5-dihydroisothiochromeno[4,3-c]pyrazol-3-yl)(3,3-dimethylmorpholino)methanone (0.35 g, 600.01 μmol, 1 eq), 5-(4,4,5,5-tetramethyl-1,3,2-dioxaborolan-2-yl)nicotinonitrile (138.04 mg, 600.01 μmol, 1 eq), K₂CO₃ (165.85 mg, 1.20 mmol, 2 eq) and Pd(dppf)C₁₂ (43.90 mg, 60.00 μmol, 0.1 eq) in dioxane (5 mL) and H₂O (0.5 mL) was degassed and purged with N₂ for 2 times and then heated to 60° C. for 16 hours under N₂. The mixture was diluted with EtOAc (40 mL) and then filtered. The filtrate was concentrated to get the residue. The residue was purified by flash silica gel chromatography (ISCO®; 10 g SepaFlash® Silica Flash Column, Eluent of 0˜40% Ethyl acetate/Petroleum ethergradient @40 mL/min). Compound 5-(1-(3,5-dichlorophenyl)-3-(3,3-dimethylmorpholine-4-carbonyl)-7-methoxy-1,5-dihydroisothiochromeno[4,3-c]pyrazol-8-yl)nicotinonitrile (0.2 g, 329.75 μmol, 55% yield) was obtained as a yellow solid. LCMS (ESI): m/z [M+H] calcd for C₃₀H₂₆Cl₂N₅O₃S: 606.11; found: 606.2.

### Synthesis of 5-[1-(3,5-dichlorophenyl)-3-(3,3-dimethylmorpholine-4-carbonyl)-7-methoxy-5H-isothiochromeno[4,3-c]pyrazol-8-yl]pyridine-3-carboxamide

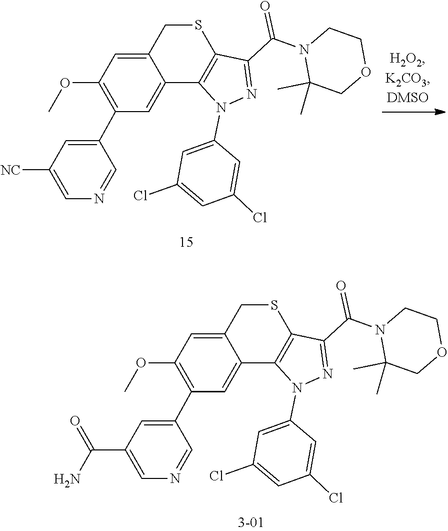 [SMILES: COc1cc2c(cc1-c1cncc(C#N)c1)-c1c(c(C(=O)N3CCOCC3(C)C)nn1-c1cc(Cl)cc(Cl)c1)SC2 ; COc1cc2c(cc1-c1cncc(C(N)=O)c1)-c1c(c(C(=O)N3CCOCC3(C)C)nn1-c1cc(Cl)cc(Cl)c1)SC2]

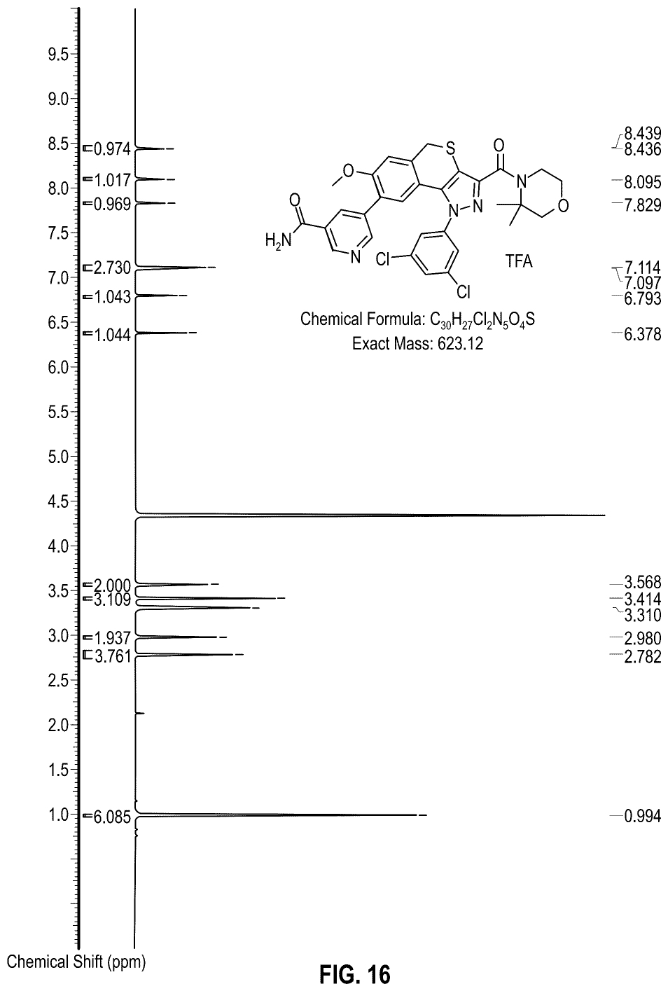

To a solution of 5-(1-(3,5-dichlorophenyl)-3-(3,3-dimethylmorpholine-4-carbonyl)-7-methoxy-1,5-dihydroisothiochromeno[4,3-c]pyrazol-8-yl)nicotinonitrile (0.2 g, 329.75 μmol, 1 eq) in DMSO (3 mL) was added K₂CO₃ (136.72 mg, 989.25 μmol, 3 eq) and H₂O₂ (0.420 g, 3.70 mmol, 355.93 μL, 30% purity, 11.23 eq) at 0° C. The mixture was stirred at 0-25° C. for 2 hr. The reaction was quenched with sat. Na₂SO₃ (10 mL). The aqueous phase was extracted with ethyl acetate (20 mL*3). The combined organic phases were washed with brine (50 mL), dried with anhydrous Na₂SO₄, It was filtered and the filtrate was concentrated in vacuum to get the residue. The residue was purified by prep-HPLC (column: Phenomenex Luna C18 150*25 mm*10 um; mobile phase: [water (TFA) -ACN]; gradient: 42%-72% B over min). Compound 5-[1-(3,5-dichlorophenyl)-3-(3,3-dimethylmorpholine-4-carbonyl)-7-methoxy-5H-isothiochromeno[4,3-c]pyrazol-8-yl]pyridine-3-carboxamide (160 mg, 216.64 μmol, 66% yield, TFA) was obtained as a white solid. LCMS (ESI): m/z [M+H] calcd for C₃₀H₂₈Cl₂N₅O₄S: 624.12.; found: 624.2. ¹H NMR (400 MHz, METHANOL-d₄) δ=8.44 (d, J=1.1 Hz, 1H), 8.09 (s, 1H), 7.83 (s, 1H), 7.14-7.08 (m, 3H), 6.79 (s, 1H), 6.38 (s, 1H), 3.57 (s, 2H), 3.41 (s, 3H), 2.98 (s, 2H), 2.79-2.77 (m, 4H), 0.99 (s, 6H). FIG. 16 shows the nuclear magnetic resonance of Compound 3-01.

### Synthesis of (1-(3,5-dichlorophenyl)-7-methoxy-8-(1-methyl-1H-pyrazol-3-yl)-1,5-dihydroisothiochromeno[4,3-c]pyrazol-3-yl)(3,3-dimethylmorpholino)methanone

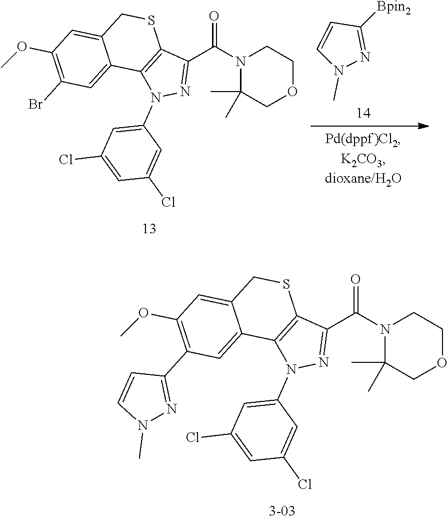

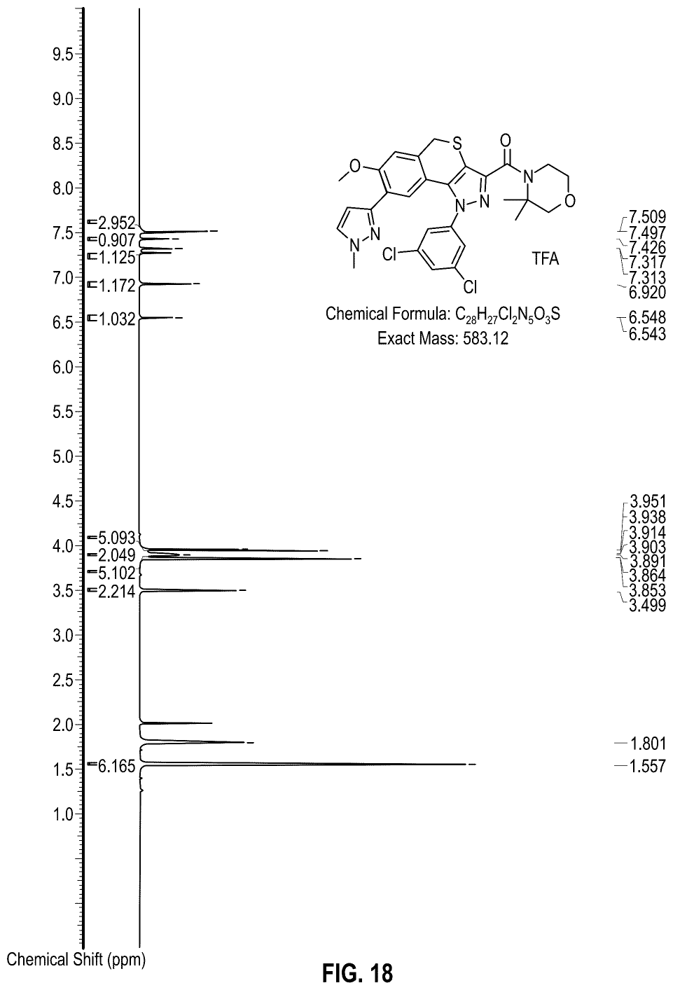

To a mixture of (8-bromo-1-(3,5-dichlorophenyl)-7-methoxy-1,5-dihydroisothiochromeno[4,3-c]pyrazol-3-yl)(3,3-dimethylmorpholino)methanone (0.25 g, 428.58 μmol, 1 eq), 1-methyl-3-(4,4,5,5-tetramethyl-1,3,2-dioxaborolan-2-yl)pyrazole (89.17 mg, 428.58 μmol, 1 eq), K₂CO₃ (118.46 mg, 857.16 μmol, 2 eq) and Pd(dppf)Cl₂ (31.36 mg, 42.86 μmol, 0.1 eq) in dioxane (5 mL) and H₂O (0.5 mL) was de-gassed and purged with N₂ for 3 times and then heated to 60° C. for 16 hours under N₂. The residue was diluted with EtOAc (40 mL) and then filtered. The filtrate was concentrated to get the residue. The residue was purified by flash silica gel chromatography (ISCO®; 10 g SepaFlash® Silica Flash Column, Eluent of 0˜40% Ethyl acetate/Petroleum ether gradient @40 mL/min). Compound [1-(3,5-dichlorophenyl)-7-methoxy-8-(1-methylpyrazol-3-yl)-5H-isothiochromeno[4,3-c]pyrazol-3-yl]-(3,3-dimethylmorpholin-4-yl)methanone (220 mg, 376.38 μmol, 88% yield) was obtained as a white solid. LCMS (ESI): m/z [M+H] calcd for C₂₈H₂₈Cl₂N₅O₃S: 584.12.11; found: 584.2. ¹H NMR (400 MHz, CHLOROFORM-d) δ=7.54-7.48 (m, 3H), 7.43 (s, 1H), 7.31 (d, J=2.0 Hz, 1H), 6.92 (s, 1H), 6.55 (d, J=2.1 Hz, 1H), 3.97-3.93 (m, 5H), 3.92-3.88 (m, 2H), 3.88-3.83 (m, 5H), 3.50 (s, 2H), 1.56 (s, 6H). FIG. 18 shows the nuclear magnetic resonance of Compound 3-03.

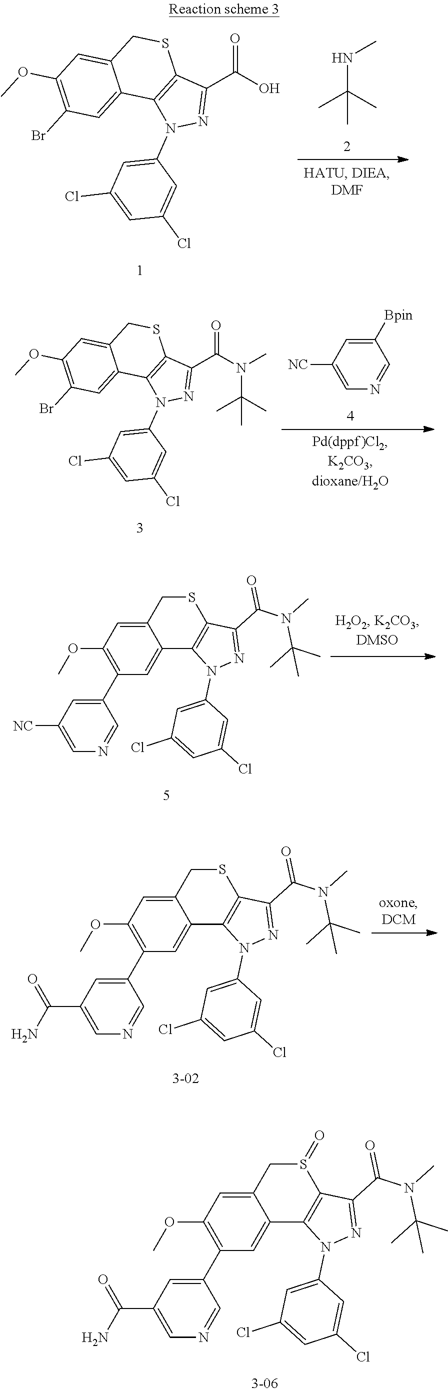

### Synthesis of 8-bromo-N-tert-butyl-1-(3,5-dichlorophenyl)-7-methoxy-N-methyl-5H-isothiochromeno[4,3-c]pyrazole-3-carboxamide

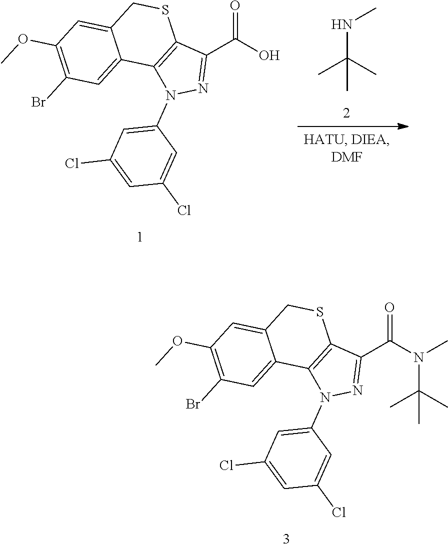

To a solution of 8-bromo-1-(3, 5-dichlorophenyl)-7-methoxy-5H-isothiochromeno[4, 3-c]pyrazole-3-carboxylic acid (598.53 mg, 1.23 mmol, 1 eq) in THF (10 mL) was added HATU (702.17 mg, 1.85 mmol, 1.5 eq) and DIEA (477.34 mg, 3.69 mmol, 643.32 μL, 3 eq) at 25° C. After addition, the mixture was stirred at 25° C. for 0.5 hr, and then N, 2-dimethylpropan-2-amine (118.04 mg, 1.35 mmol, 162.37 μL, 1.1 eq) in THF (5 mL) was added at 25° C. The resulting mixture was stirred at 25° C. for 16 hr. The mixture was poured into ice-water (30 mL) and stirred for 5 min. The aqueous phase was extracted with ethyl acetate (20 mL*3). The combined organic phases were washed with brine (50 mL), dried with anhydrous Na₂SO₄, It was filtered and the filtrate was concentrated in vacuum to get the residue. The residue was purified by flash silica gel chromatography (ISCO®; 10 g SepaFlash® Silica Flash Column, Eluent of 0˜20% Ethyl acetate/Petroleum ethergradient @40 mL/min). Compound 8-bromo-N-tert-butyl-1-(3,5-dichlorophenyl)-7-methoxy-N-methyl-5H-isothiochromeno[4,3-c]pyrazole-3-carboxamide (0.65 g, 1.17 mmol, 95% yield) was obtained as a yellow oil. LCMS (ESI): m/z [M+H] calcd for C₂₃H₂₂BrCl₂N₃O₂S: 554.00; found: 554.1.

### Synthesis of N-tert-butyl-8-(5-cyano-3-pyridyl)-1-(3,5-dichlorophenyl)-7-methoxy-N-methyl-5H-isothiochromeno [4,3-c]pyrazole-3-carboxamide

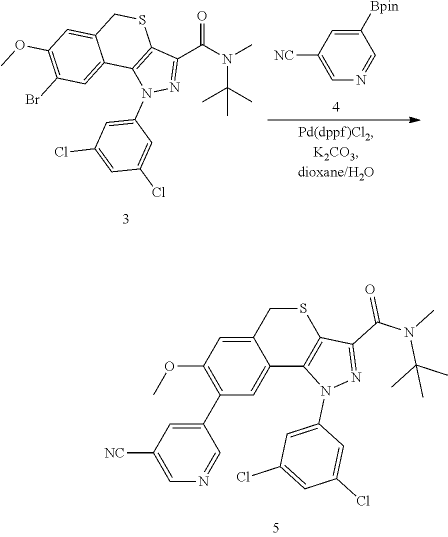

A mixture of 8-bromo-N-tert-butyl-1-(3,5-dichlorophenyl)₇-methoxy-N-methyl-5H-isothiochromeno[4,3-c]pyrazole-3-carboxamide (0.4 g, 720.31 μmol, 1 eq), 5-(4,4,5,5-tetramethyl-1,3, 2-dioxaborolan-2-yl)pyridine-3-carbonitrile (165.72 mg, 720.31 μmol, 1 eq), K₂CO₃ (199.10 mg, 1.44 mmol, 2 eq) and Pd(dppf)Cl₂ (52.71 mg, 72.03 μmol, 0.1 eq) in dioxane (5 mL) and H₂O (0.5 mL) was degassed and purged with N₂ for 3 times and then heated to 60° C. for 16 hours under N₂. The mixture was diluted with EtOAc (40 mL) and then filtered. The filtrate was concentrated to get the residue. The residue was purified by flash silica gel chromatography (ISCO®; 10 g SepaFlash® Silica Flash Column, Eluent of 0˜40% Ethyl acetate/Petroleum ethergradient @40 mL/min). Compound N-tert-butyl-8-(5-cyano-3-pyridyl)-1-(3,5-dichlorophenyl)-7-methoxy-N-methyl-5H-isothiochromeno[4, 3-c]pyrazole-3-carboxamide (0.4 g, 691.43 μmol, 96% yield) was obtained as a yellow solid. LCMS (ESI): m/z [M+H] calcd for C₂₉H₂₅Cl₂N₅O₂S: 578.10; found: 578.1.

### Synthesis of N-tert-butyl-8-(5-carbamoyl-3-pyridyl)-1-(3,5-dichlorophenyl)-7-methoxy-N-methyl-5H-isothiochromeno[4,3-c]pyrazole-3-carboxamide

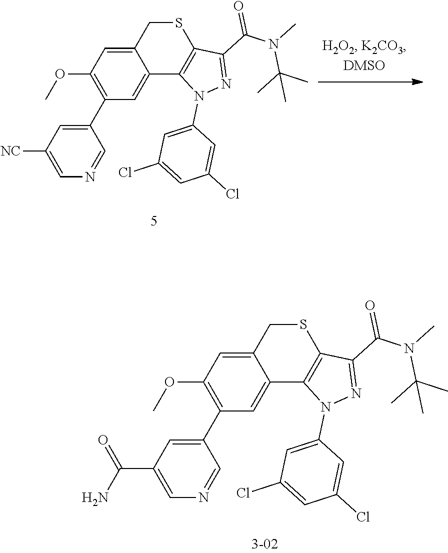 [SMILES: COc1cc2c(cc1-c1cncc(C#N)c1)-c1c(c(C(=O)N(C)C(C)(C)C)nn1-c1cc(Cl)cc(Cl)c1)SC2 ; COc1cc2c(cc1-c1cncc(C(N)=O)c1)-c1c(c(C(=O)N(C)C(C)(C)C)nn1-c1cc(Cl)cc(Cl)c1)SC2]

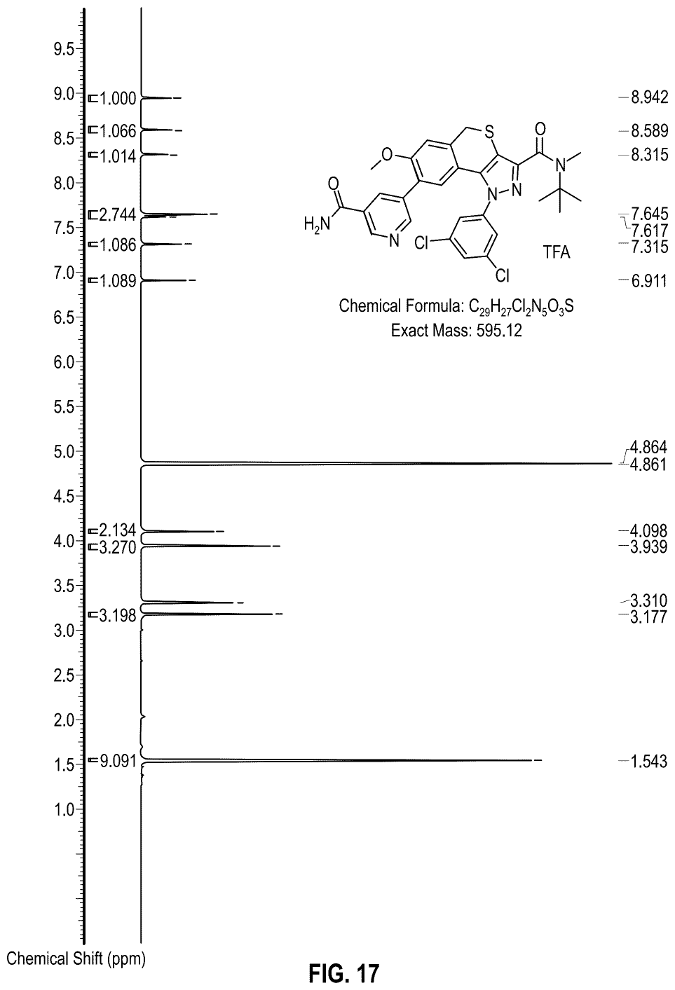

To a solution of N-tert-butyl-8-(5-cyano-3-pyridyl)-1-(3,5-dichlorophenyl)-7-methoxy-N-methyl-5H-isothiochromeno[4,3-c]pyrazole-3-carboxamide (0.4 g, 691.43 μmol, 1 eq) in DMSO (5 mL) was added K₂CO₃ (286.68 mg, 2.07 mmol, 3 eq) and H₂O₂ (0.790 g, 6.97 mmol, 669.49 μL, 30% purity, 10.08 eq) at 0° C. The mixture was stirred at 0-25° C. for 2 hr. The reaction was quenched with sat. Na₂SO₃ (10 mL). The aqueous phase was extracted with ethyl acetate (20 mL*3). The combined organic phase was washed with brine (50 mL), dried with anhydrous Na₂SO₄, It was filtered and the filtrate was concentrated in vacuum to get the residue. The residue was purified by prep-HPLC (column: Phenomenex Luna C18 150*25 mm*10 um; mobile phase: [water (TFA) -ACN]; gradient: 50%-80% B over min). Compound N-tert-butyl-8-(5-carbamoyl-3-pyridyl) -1-(3,5-dichlorophenyl)-7-methoxy-N-methyl-5H-isothiochromeno[4,3-c]pyrazole-3-carboxamide (160 mg, 225.18 μmol, 33% yield, TFA) was obtained as a white solid. LCMS (ESI): m/z [M+H] calcd for C₂₉H₂₇Cl₂N₅O₃S: 596.12; found: 596.2. ¹H NMR (400 MHz, METHANOL-d₄) δ=8.94 (s, 1H), 8.59 (s, 1H), 8.32 (d, J=1.0 Hz, 1H), 7.67-7.59 (m, 3H), 7.31 (s, 1H), 6.91 (s, 1H), 4.10 (s, 2H), 3.94 (s, 3H), 3.18 (s, 3H), 1.54 (s, 9H). FIG. 17 shows the nuclear magnetic resonance of Compound 3-02.

### Synthesis of N-tert-butyl-8-(5-carbamoyl-3-pyridyl)-1-(3,5-dichlorophenyl)-7-methoxy-N-methyl-4-oxo-5H-isothiochromeno[4,3-c]pyrazole-3-carboxamide

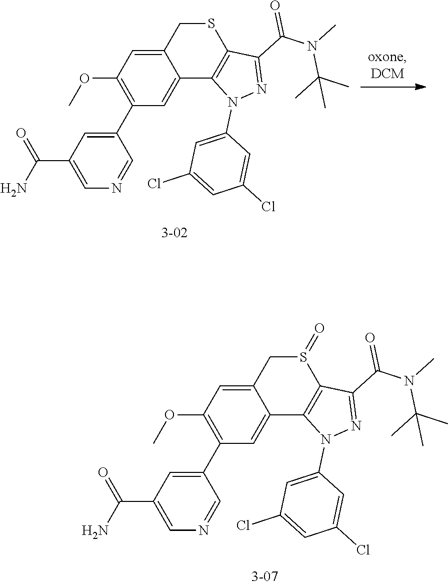 [SMILES: COc1cc2c(cc1-c1cncc(C(N)=O)c1)-c1c(c(C(=O)N(C)C(C)(C)C)nn1-c1cc(Cl)cc(Cl)c1)SC2 ; COc1cc2c(cc1-c1cncc(C(N)=O)c1)-c1c(c(C(=O)N(C)C(C)(C)C)nn1-c1cc(Cl)cc(Cl)c1)S(=O)C2]

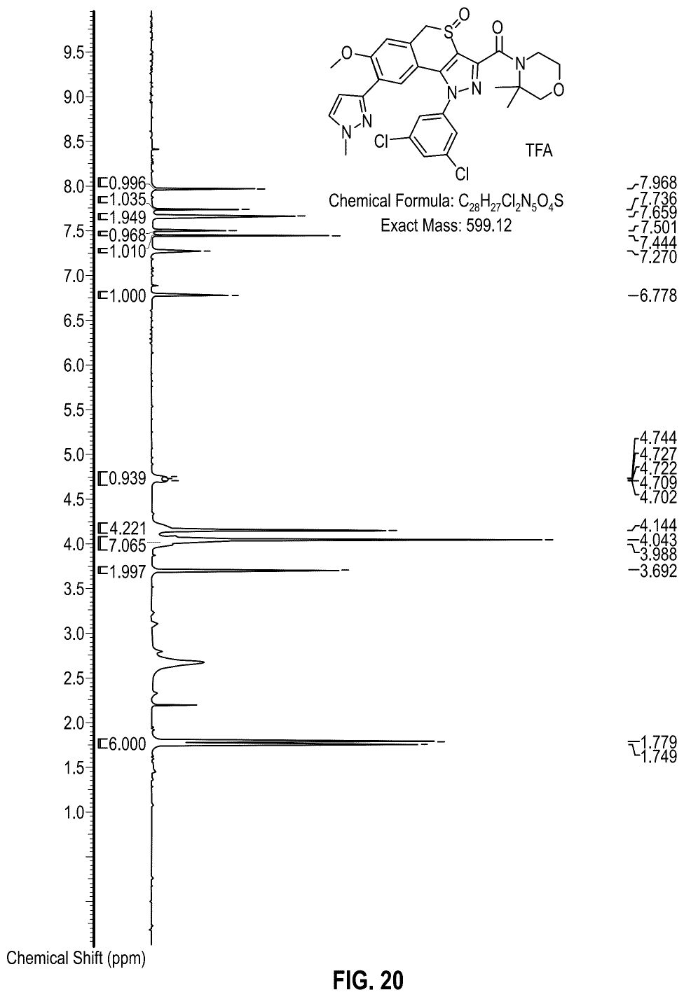

To a solution of N-tert-butyl-8-(5-carbamoyl-3-pyridyl)-1-(3,5-dichlorophenyl)-7-methoxy-N-methyl-5H-isothiochromeno[4,3-c]pyrazole-3-carboxamide (20 mg, 33.53 μmol, 1 eq) in DCM (5 mL) was added Oxone (82.45 mg, 134.11 μmol, 4 eq). The mixture was stirred at 25° C. for 16 hr. The reaction was quenched with sat. Na₂SO₃ (10 mL). The aqueous phase was extracted with ethyl acetate (10 mL*2). The combined organic phases were washed with brine (20 mL), dried with anhydrous Na₂SO₄, It was filtered and the filtrate was concentrated in vacuum to get the residue. The residue was purified by prep-HPLC (column: Phenomenex Luna C18 150*25 mm*10 um; mobile phase: [water (TFA) -ACN]; gradient: 38%-68% B over 9 min). Compound N-tert-butyl-8-(5-carbamoyl-3-pyridyl)-1-(3,5-dichlorophenyl)-7-methoxy-N-methyl-4-oxo-5H-isothiochromeno[4, 3-c]pyrazole-3-carboxamide (10 mg, 13.76 μmol, 41% yield, TFA) was obtained as a white solid. LCMS (ESI): m/z [M+H] calcd for C₂₉H₂₇Cl₂N₅O₄S: 612.12; found: 612.2. ¹H NMR (400 MHz, CHLOROFORM-d) δ=8.92 (s, 1H), 8.57 (s, 1H), 8.02-7.97 (m, 1H), 7.56 (t, J=1.8 Hz, 1H), 7.49 (d, J=1.7 Hz, 2H), 7.18 (s, 1H), 7.02 (s, 1H), 4.58 (d, J=15.4 Hz, 1H), 4.03 (d, J=15.3 Hz, 1H), 3.89 (s, 3H), 3.21 (s, 3H), 1.57 (s, 9H). FIG. 20 shows the nuclear magnetic resonance of Compound 3-07.

### Synthesis of N-tert-butyl-1-(3,5-dichlorophenyl)-7-methoxy-N-methyl-8-(1-methylpyrazol-3-yl)-5H-isothiochromeno[4,3-c]pyrazole-3-carboxamide

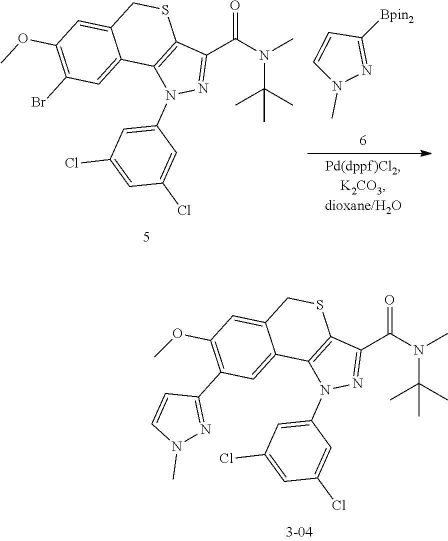

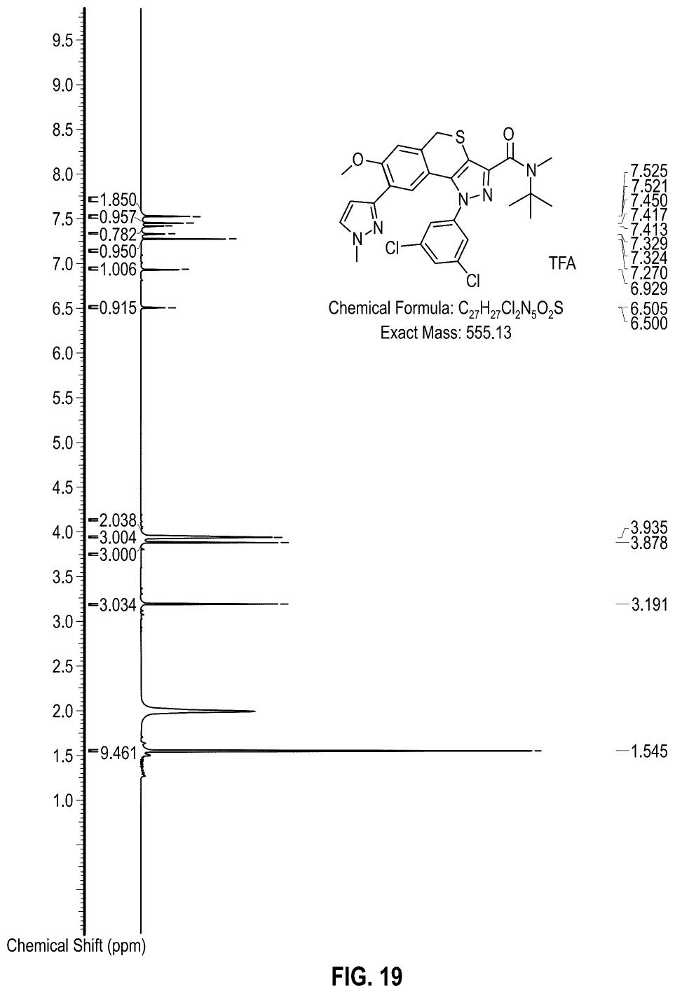

8-bromo-N-tert-butyl-1-(3,5-dichlorophenyl)-7-methoxy-N-methyl-5H-isothiochromeno[4,3-c]pyrazole-3-carboxamide (0.25 g, 450.20 μmol, 1 eq), 1-methyl-3-(4,4,5, 5-tetramethyl-1, 3, 2-dioxaborolan-2-yl) pyrazole (93.67 mg, 450.20 μmol, 1 eq), K₂CO₃ (124.44 mg, 900.39 μmol, 2 eq) and Pd(dppf)Cl₂ (32.94 mg, 45.02 μmol, 0.1 eq) in dioxane (5. ML) and H₂O (0.5 mL) was degassed and purged with N₂ for 3 times and then heated to 60° C. for 16 hours under N₂. The residue was diluted with EtOAc (40 mL) and then filtered. The filtrate was concentrated to get the residue. The residue was purified by flash silica gel chromatography (ISCO®; 10 g SepaFlash® Silica Flash Column, Eluent of 0˜40% Ethyl acetate/Petroleum ethergradient @40 mL/min). Compound N-tert-butyl-1-(3,5-dichlorophenyl)-7-methoxy-N-methyl-8-(1-methylpyrazol-3-yl)-5H-isothiochromeno[4,3-c]pyrazole-3-carboxamide (0.2 g, 359.39 μmol, 80% yield) was obtained as a white solid. LCMS (ESI): m/z [M+H] calcd for C₂₇H₂₇Cl₂N₅O₂S: 556.13; found: 556.3. ¹H NMR (400 MHz, CHLOROFORM-d) δ=7.52 (d, J=1.7 Hz, 2H), 7.45 (s, 1H), 7.42 (d, J=1.7 Hz, 1H), 7.33 (d, J=2.2 Hz, 1H), 6.93 (s, 1H), 6.50 (d, J=2.1 Hz, 1H), 3.96-3.95 (m, 2H), 3.94 (s, 3H), 3.88 (s, 3H), 3.19 (s, 3H), 1.54 (s, 9H). FIG. 19 shows the nuclear magnetic resonance of Compound 3-04.

### Example 3

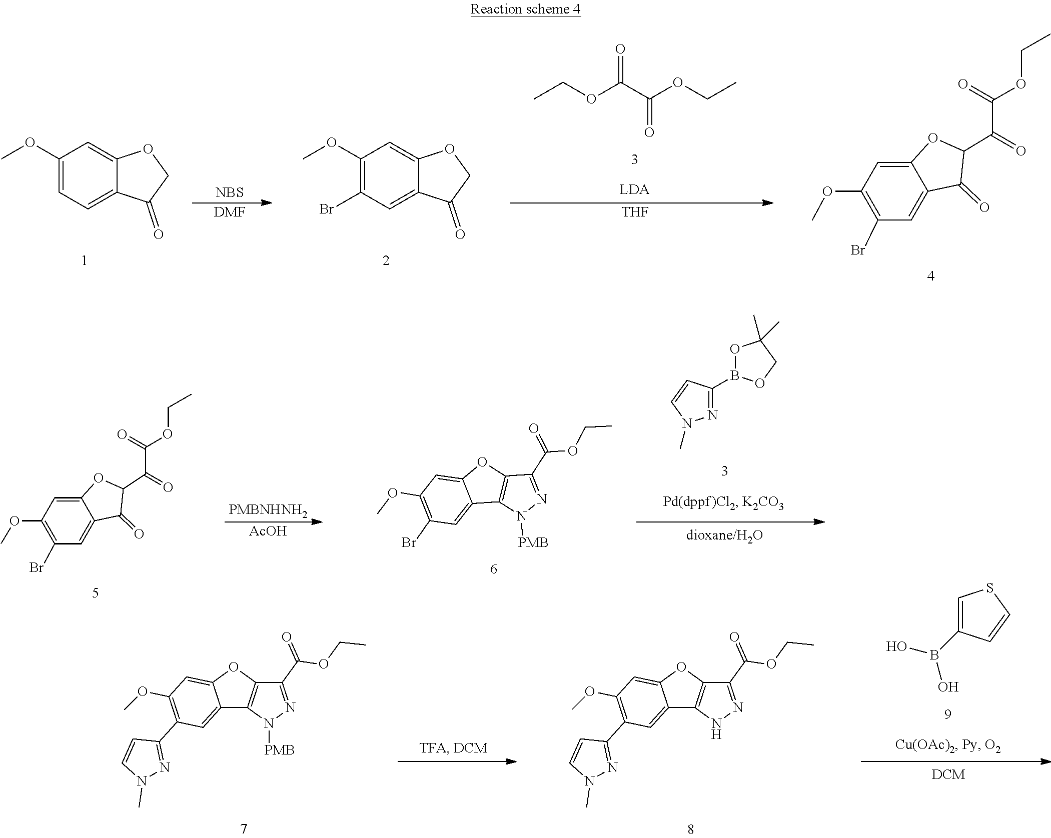

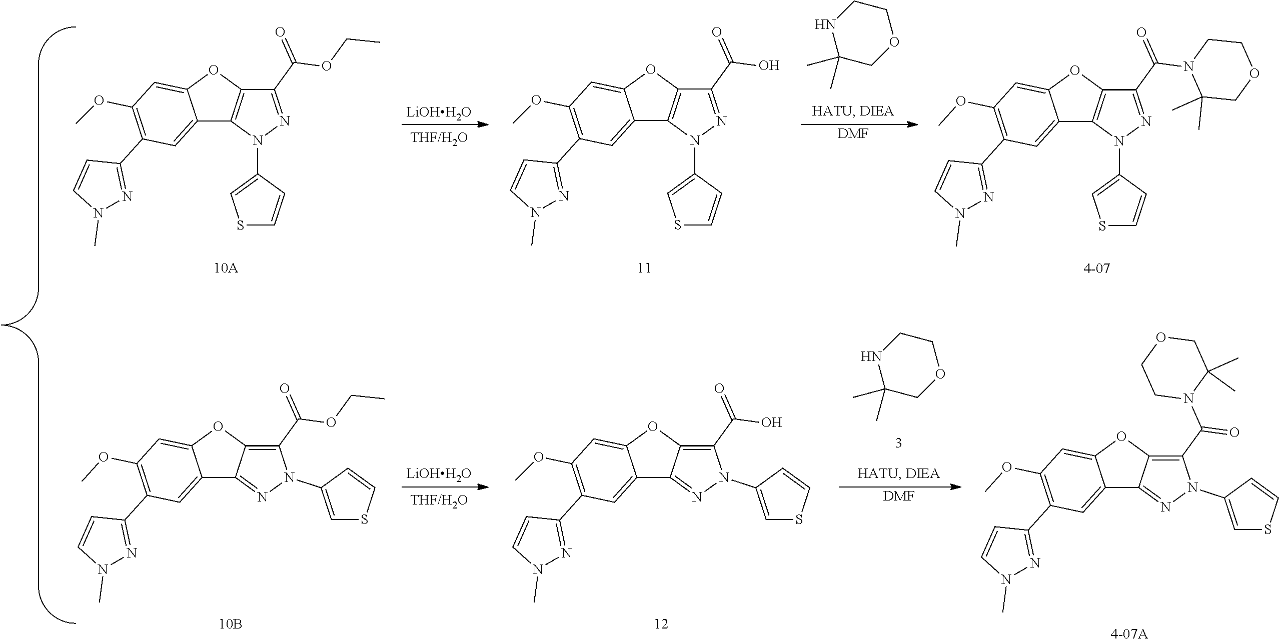

### Synthesis of 5-bromo-6-methoxy-benzofuran-3-one

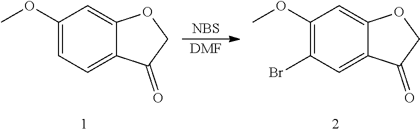

To a mixture of 6-methoxybenzofuran-3(2H)-one (12.2 g, 74.32 mmol, 1 eq) in DMF (60 mL) was added NBS (15.87 g, 89.18 mmol, 1.2 eq) at 0° C. The mixture was stirred at 15° C. for 20 h. The mixture was poured into H₂O (600 mL), There were precipitates formed in the reaction mixture, the resulting mixture was filtered, the filter cake was dried under vacuum to afford 5-bromo-6-methoxybenzofuran-3(2H)-one (16.5 g, 61.78 mmol, 83% yield, 91% purity) as a brown solid. ¹H NMR (400 MHz, DMSO-d₆) δ=7.81 (s, 1H), 7.08-6.97 (m, 1H), 4.83 (s, 2H), 3.96 (s, 3H).

### Synthesis of 4: ethyl 2-(5-bromo-6-methoxy-3-oxo-2,3-dihydrobenzofuran-2-yl)-2-oxoacetate

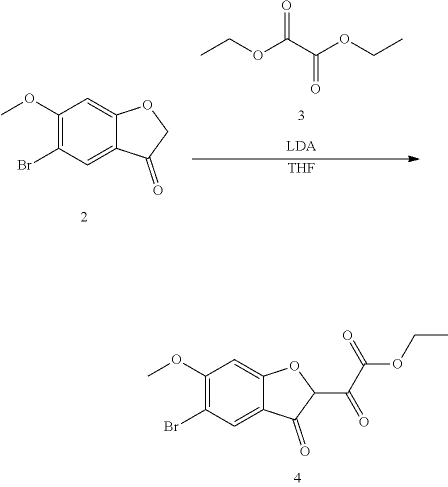

To a mixture of 5-bromo-6-methoxybenzofuran-3(2H)-one (19.1 g, 78.58 mmol, 1 eq) in THF (150 mL) was added LDA (2 M, 47.15 mL, 1.2 eq) at −78° C. under N₂ atmosphere. The mixture was stirred at −78° C. for 15 min, then diethyl oxalate (18.37 g, 125.73 mmol, 17.17 mL, 1.6 eq) was added. The mixture was stirred at 0° C. for 1 h. The reaction mixture was quenched by 1N HCl (100 mL) and then extracted with ethyl acetate (100 mL*3). The combined organic phases were washed with brine (200 mL) and dried over Na₂SO₄. It was filtered and the filtrate was concentrated under vacuum to afford ethyl 2-(5-bromo-6-methoxy-3-oxo-2,3-dihydrobenzofuran-2-yl)-2-oxoacetate (35 g, crude) as a brown solid. ¹H NMR (400 MHz, DMSO-d₆) δ=8.26 (s, 1H), 7.38 (s, 1H), 4.34 (q, J=7.09 Hz, 2H), 3.94 (s, 3H), 1.30 (t, J=7.09 Hz, 3H).

### Synthesis of ethyl 7-bromo-6-methoxy-1-(4-methoxybenzyl)-1H-benzofuro[3,2-c]pyrazole-3-carboxylate

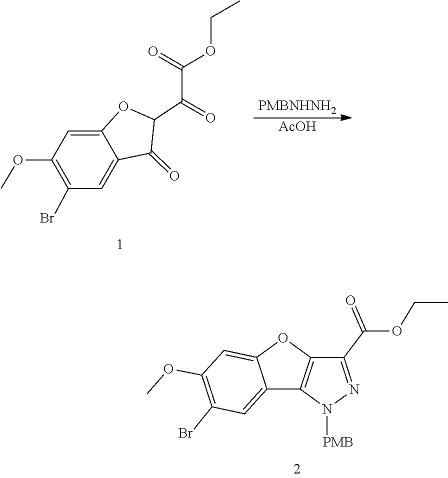

To a mixture of ethyl 2-(5-bromo-6-methoxy-3-oxo-2,3-dihydrobenzofuran-2-yl)-2-oxoacetate (300 mg, 874.31 μmol, 1 eq) in AcOH (2 mL) was added (4-methoxyphenyl)methylhydrazine;hydrochloride (164.94 mg, 874.31 μmol, 1 eq), the mixture was stirred at 110° C. for 1 h. The pH of the reaction mixture was adjusted to 7 with sat. NaHCO₃, the resulting mixture was extracted with ethyl acetate (50 mL*3). The combined organic phases were concentrated under vacuum to give a residue. The residue was purified by flash silica gel chromatography column (petroleum ether: ethyl acetate=1:1) to afford ethyl 7-bromo-6-methoxy-1-(4-methoxybenzyl)-1H-benzofuro[3,2-c]pyrazole-3-carboxylate (150 mg, 326.59 μmol, 37% yield) as a brown solid.

### Synthesis of ethyl 6-methoxy-7-(1-methylpyrazol-3-yl)-1H-benzofuro[3,2-c]pyrazole-3-carboxylate

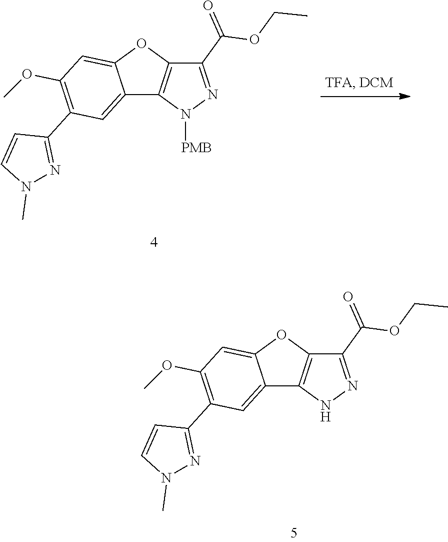

To a mixture of ethyl 6-methoxy-1-[(4-methoxyphenyl)methyl]-7-(1-methylpyrazol-3-yl)benzofuro[3,2-c]pyrazole-3-carboxylate (650 mg, 1.41 mmol, 1 eq) a in DCM (5 mL) was added TFA (160.95 mg, 1.41 mmol, 104.85 μL, 1 eq), the mixture was stirred at 70° C. for 16 h. The mixture was concentrated under vacuum to give a residue. The residue was purified by flash silica gel chromatography column (petroleum ether: ethyl acetate=5:1˜1:1) to afford ethyl 6-methoxy-7-(1-methylpyrazol-3-yl)-1H-benzofuro[3,2-c]pyrazole-3-carboxylate (420 mg, 1.23 mmol, 87% yield) as a white solid.

### Synthesis of ethyl 6-methoxy-7-(1-methylpyrazol-3-yl)-1-(3-thienyl)benzofuro[3,2-c]pyrazole-3-carboxylate and ethyl 6-methoxy-7-(1-methylpyrazol-3-yl)-2-(3-thienyl)benzofuro[3,2-c]pyrazole-3-carboxylate

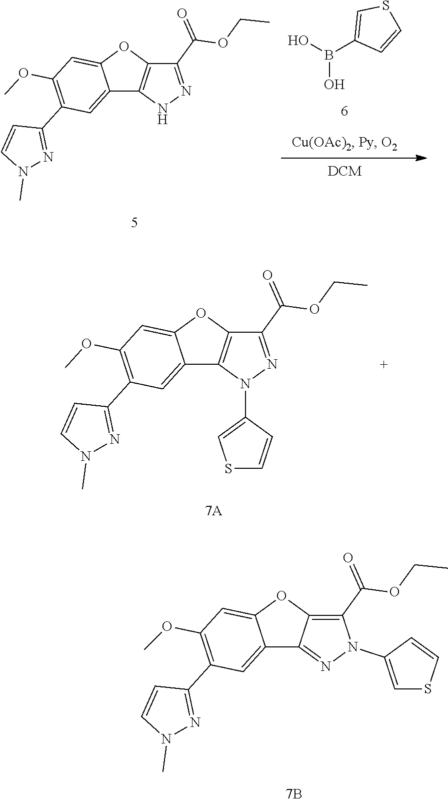

To a mixture of ethyl 6-methoxy-7-(1-methylpyrazol-3-yl)-1H-benzofuro[3,2-c]pyrazole-3-carboxylate (320 mg, 940.26 μmol, 1 eq) and 3-thienylboronic acid (180.47 mg, 1.41 mmol, 1.5 eq) in DCM (10 mL) was added Cu(OAc)₂ (170.78 mg, 940.26 mol, 1 eq), Py (148.75 mg, 1.88 mmol, 151.78 μL, 2 eq), 4A MS (50 mg), the mixture was stirred at 25° C. for 16 h under O₂ (15 psi) atmosphere. H₂O (20 mL) and NH₃·H₂O(1 mL) was added, the resulting mixture was extracted with ethyl acetate (20 mL*3). The combined organic phases were concentrated under vacuum to give a residue. The residue was purified by flash silica gel chromatography column(petroleum ether: ethyl acetate=2:1), the residue was purified by prep-TLC (petroleum ether: ethyl acetate=2:1)(3rd purification) to afford ethyl 6-methoxy-7-(1-methylpyrazol-3-yl)-1-(3-thienyl)benzofuro[3,2-c]pyrazole-3-carboxylate (23 mg, 54.44 μmol, 6% yield) as a brown solid. 1H NMR (400 MHz, DMSO-d6) δ=8.18 (s, 1H), 7.63 (dd, J=1.3, 3.2 Hz, 1H), 7.58 (dd, J=3.2, 5.1 Hz, 1H), 7.49-7.41 (m, 1H), 7.35 (d, J=2.0 Hz, 1H), 7.21 (s, 1H), 6.74 (d, J=2.0 Hz, 1H), 4.47 (q, J=7.2, 2H), 3.91 (s, 6H), 1.42 (t, J=7.2, 3H).

Ethyl 6-methoxy-7-(1-methylpyrazol-3-yl)-2-(3-thienyl)benzofuro[3,2-c]pyrazole-3-carboxylate (180 mg, 426.08 μmol, 45% yield) was obtained as a brown solid. ¹H NMR (400 MHz, DMSO-d6) δ=8.43 (s, 1H), 7.56 (dd, J=1.3, 3.2 Hz, 1H), 7.49-7.44 (m, 1H), 7.43-7.40 (m, 1H), 7.39-7.35 (m, 1H), 7.21 (s, 1H), 6.74 (d, J=2.2 Hz, 1H), 4.43 (q, J=7.2, 2H), 3.98 (s, 6H), 1.41 (t, J=7.2, 3H).

### Synthesis of 6-methoxy-7-(1-methylpyrazol-3-yl)-1-(3-thienyl)benzofuro[3,2-c]pyrazole-3-carboxylic acid

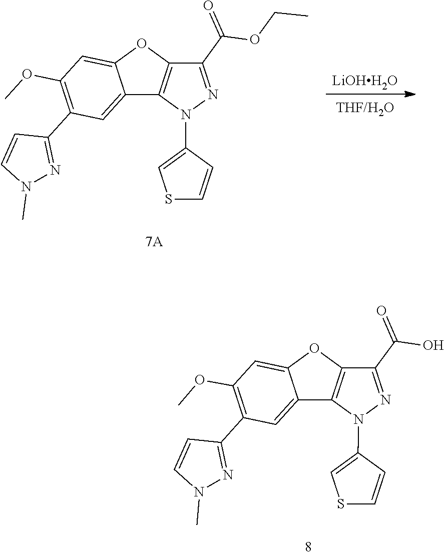 [SMILES: CCOC(=O)c1nn(-c2ccsc2)c2c1oc1cc(OC)c(-c3ccn(C)n3)cc12 ; "LiOH·H2O" ; COc1cc2oc3c(C(=O)O)nn(-c4ccsc4)c3c2cc1-c1ccn(C)n1]

To a mixture of ethyl 6-methoxy-7-(1-methylpyrazol-3-yl)-1-(3-thienyl)benzofuro[3,2-c]pyrazole-3-carboxylate (23 mg, 54.44 μmol, 1 eq) in H₂O (1 mL) and THF (2 mL) was added LiOH·H₂O (6.85 mg, 163.33 μmol, 3 eq), the mixture was stirred at 25° C. for 2 h. The mixture was concentrated under vacuum to remove THF. The pH of the reaction mixture was adjusted to 6 with 1 M HCl aqueous solution. There were precipitates formed in the reaction mixture, the resulting mixture was filtered and filter cake was dried under vacuum to give 6-methoxy-7-(1-methylpyrazol-3-yl)-1-(3-thienyl)benzofuro[3,2-c]pyrazole-3-carboxylic acid (20 mg, 50.71 μmol, 93% yield) as a light green solid.

### Synthesis of (3,3-dimethylmorpholin-4-yl)-[6-methoxy-7-(1-methylpyrazol-3-yl)-1-(3-thienyl)benzofuro[3,2-c]pyrazol-3-yl]methanone

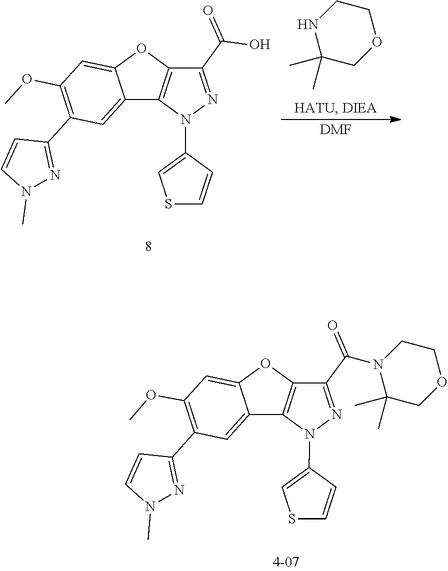

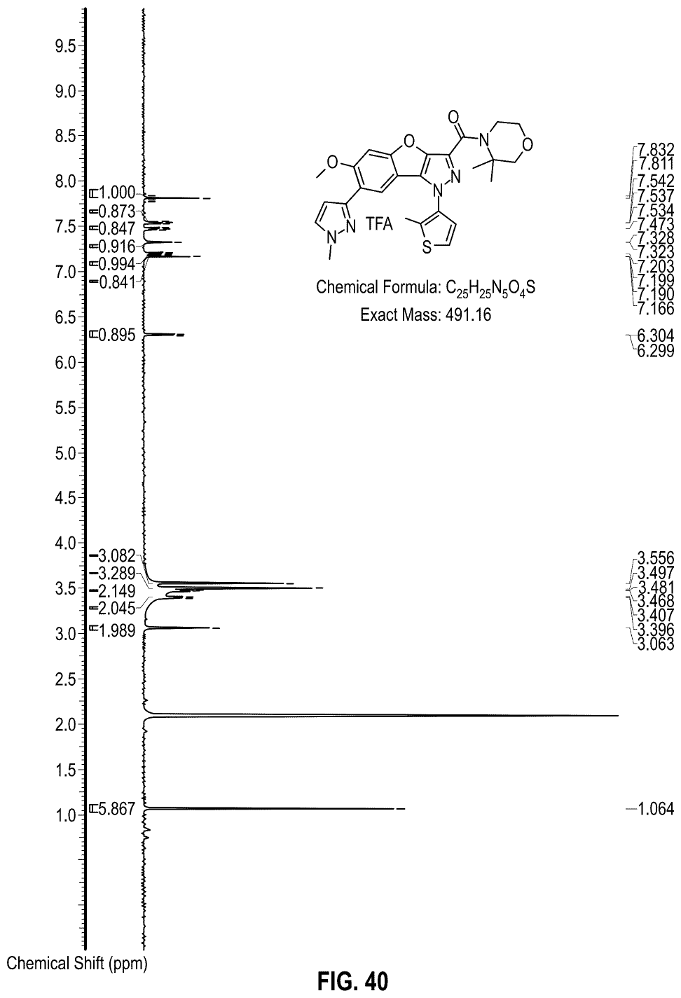

To a mixture of 6-methoxy-7-(1-methylpyrazol-3-yl)-1-(3-thienyl)benzofuro[3,2-c]pyrazole-3-carboxylic acid (20 mg, 50.71 μmol, 1 eq) in DMF (1 mL) was added HATU (28.92 mg, 76.06 μmol, 1.5 eq), DIEA (13.11 mg, 101.42 μmol, 17.66 y L, 2 eq), then 3,3-dimethylmorpholine (8.76 mg, 76.06 μmol, 1.5 eq) was added, the mixture was stirred at 25° C. for 1 h. The mixture was filtered. The filtrate was purified by prep-HPLC(column: Phenomenex Luna C18 150*25 mm*10 um;mobile phase: [water(TFA)-ACN];gradient:52%-82% B over 9 min) followed by lyophilization to afford (3,3-dimethylmorpholin-4-yl)-[6-methoxy-7-(1-methylpyrazol-3-yl)-1-(3-thienyl)benzofuro[3,2-c]pyrazol-3-yl]methanone (5.69 mg, 9.40 μmol, 19% yield, TFA) as a yellow solid.LCMS (ESI): m/z [M+H] calcd for C₂₅H₂₆N₅O₄S:492.16; found: 492.1. 1H NMR (400 MHz, DMSO-d6) δ=7.81 (s, 1H), 7.54 (dd, J=1.3, 3.2 Hz, 1H), 7.48 (dd, J=3.2, 5.1 Hz, 1H), 7.33 (d, J=2.1 Hz, 1H), 7.19 (dd, J=1.4, 5.2 Hz, 1H), 7.17 (s, 1H), 6.30 (d, J=2.2 Hz, 1H), 3.56 (s, 3H), 3.50 (s, 3H), 3.47 (br d, J=5.3 Hz, 2H), 3.40 (br d, J=4.5 Hz, 2H), 3.06 (s, 2H), 1.06 (s, 6H). FIG. 40 shows the nuclear magnetic resonance of Compound 4-07.

### Synthesis of (3,3-dimethylmorpholino)(6-methoxy-7-(1-methyl-1H-pyrazol-3-yl)-2-(thiophen-3-yl)-2H-benzofuro[3,2-c]pyrazol-3-yl)methanone

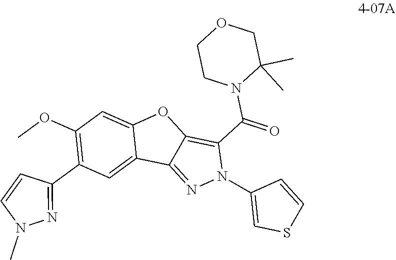 [SMILES: COc1cc2oc3c(C(=O)N4CCOCC4(C)C)n(-c4ccsc4)nc3c2cc1-c1ccn(C)n1]

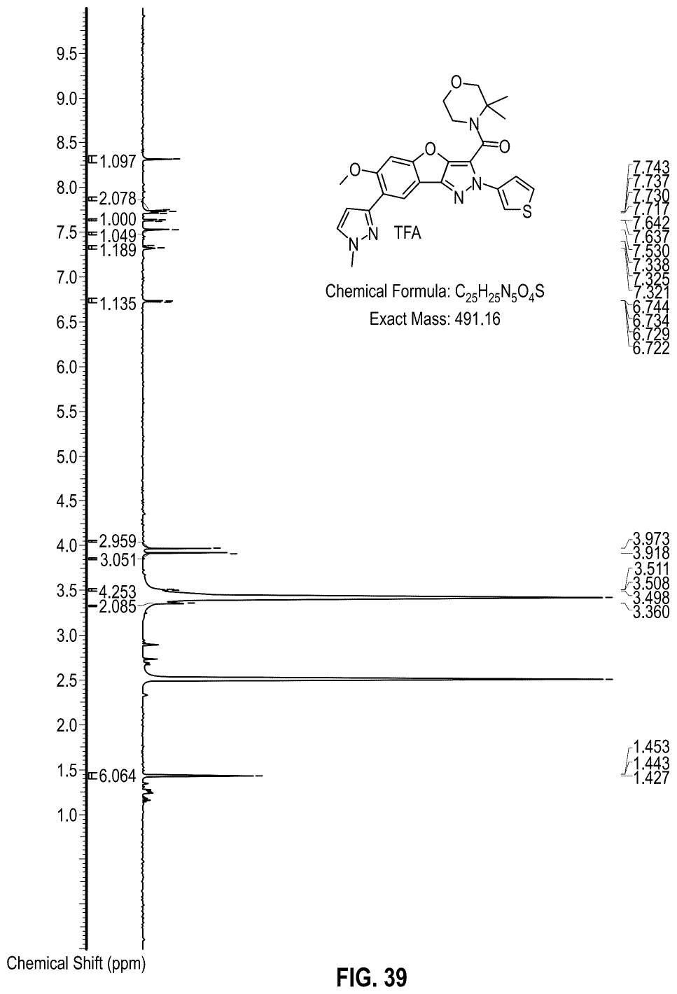

Compound (3,3-dimethylmorpholino)(6-methoxy-7-(1-methyl-1H-pyrazol-3-yl)-2-(thiophen-3-yl)-2H-benzofuro[3,2-c]pyrazol-3-yl)methanone was synthesized by the same procedure as Compound 4-07. LCMS (ESI): m/z [M+H] calcd for C₂₅H₂₆N₅O₄S:492.16; found: 492.1. 1H NMR (400 MHz, DMSO-d6) δ=8.36-8.28 (m, 1H), 7.75-7.71 (m, 2H), 7.64 (dd, J=1.4, 3.2 Hz, 1H), 7.53 (s, 1H), 7.33 (dd, J=1.4, 5.1 Hz, 1H), 6.73 (d, J=2.3 Hz, 1H), 3.97 (s, 3H), 3.92 (s, 3H), 3.52-3.49 (m, 4H), 3.36 (br s, 2H), 1.43 (s, 6H). FIG. 39 shows the nuclear magnetic resonance of Compound 4-07A.

### Example 4

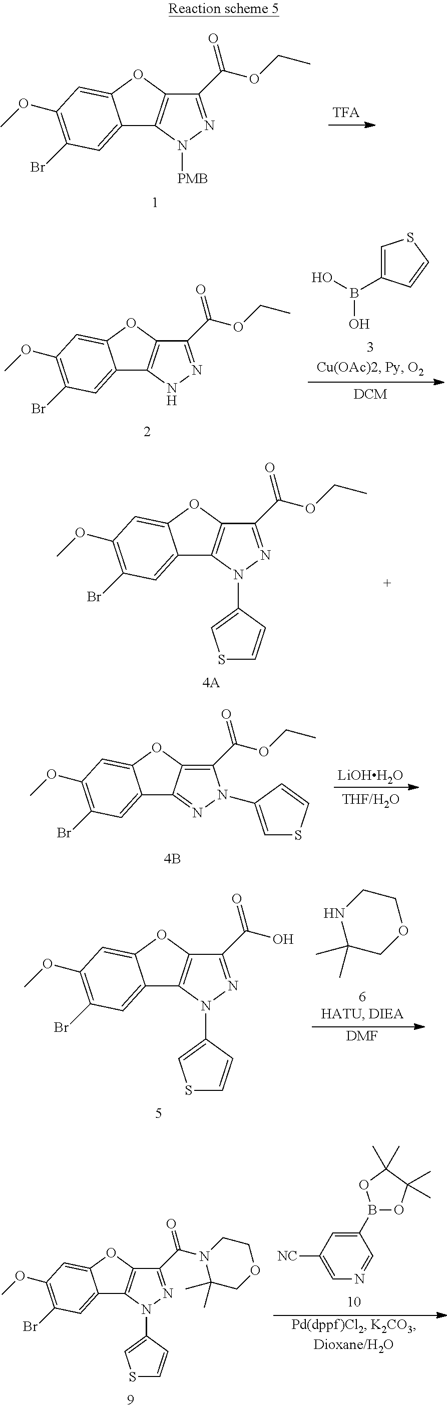

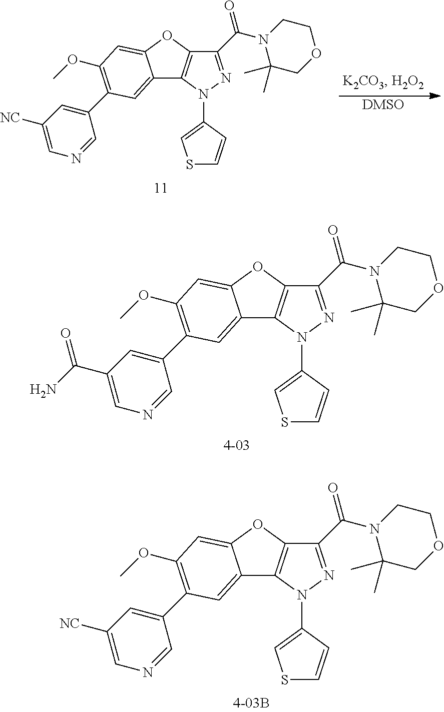

### Synthesis of 7-bromo-6-methoxy-1H-benzofuro[3,2-c]pyrazole-3-carboxylate

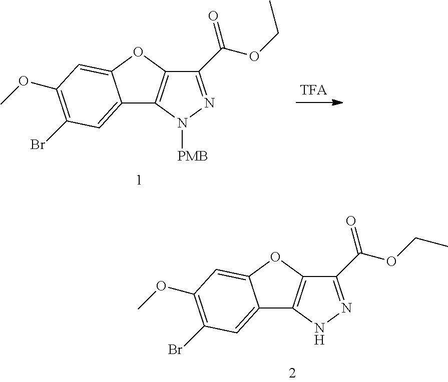

A mixture of ethyl 7-bromo-6-methoxy-1-(4-methoxybenzyl)-1H-benzofuro[3,2-c]pyrazole-3-carboxylate (200 mg, 435.46 μmol, 1 eq) in TFA (49.65 mg, 435.46 μmol, 32.35 μL, 1 eq) was stirred at 70° C. for 16 h. The mixture was concentrated under vacuum to give a residue. The residue was purified by flash silica gel chromatography column (petroleum ether: ethyl acetate=5:1-2:1) to afford ethyl 7-bromo-6-methoxy-1H-benzofuro[3,2-c]pyrazole-3-carboxylate (145 mg, 427.55 μmol, 98% yield) as a white solid.

### Synthesis of ethyl 7-bromo-6-methoxy-1-(3-thienyl)benzofuro[3,2-c]pyrazole-3-carboxylate and ethyl 7-bromo-6-methoxy-2-(3-thienyl)benzofuro[3,2-c]pyrazole-3-carboxylate

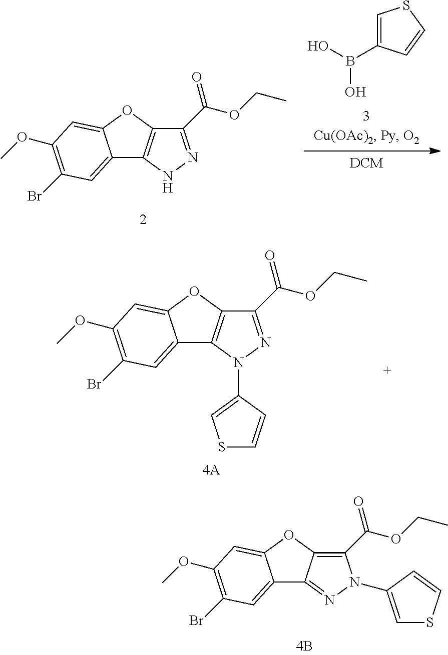

To a mixture of ethyl 7-bromo-6-methoxy-1H-benzofuro[3,2-c]pyrazole-3-carboxylate (145 mg, 427.55 μmol, 1 eq) and 3-thienylboronic acid (82.06 mg, 641.33 μmol, 1.5 eq) in DCM (10 mL) was added Cu(OAc)₂ (77.66 mg, 427.55 μmol, 1 eq), Py (67.64 mg, 855.10 μmol, 69.02 μL, 2 eq),4A MS (50 mg, 1.00 eq), the mixture was stirred at 25° C. for 16 h under 02 atmosphere (15 psi). The mixture was added H₂O (10 mL) and 25% NH₃·H₂O (0.5 mL), the resulting mixture was extracted with ethyl acetate (10 mL*3), the combined organic phases were concentrated under vacuum to give a residue. the residue was purified by flash silica gel chromatography column(petroleum ether: ethyl acetate=4:1) to afford ethyl 7-bromo-6-methoxy-2-(3-thienyl)benzofuro[3,2-c]pyrazole-3-carboxylate (68 mg, 161.42 μmol, 38% yield) as a white solid, ethyl 7-bromo-6-methoxy-1-(3-thienyl)benzofuro[3,2-c]pyrazole-3-carboxylate (40 mg, 94.95 μmol, 22% yield) as a yellow solid. 1H NMR (400 MHz, CHLOROFORM-d) δ=8.10 (s, 1H), 7.56 (dd, J=1.3, 3.1 Hz, 1H), 7.39 (dd, J=3.3, 5.1 Hz, 1H), 7.32 (dd, J=1.2, 5.2 Hz, 1H), 7.19 (s, 1H), 4.42 (q, J=7.2 Hz, 2H), 4.00 (s, 3H), 1.45-1.41 (m, 3H).

Ethyl 7-bromo-6-methoxy-2-(3-thienyl)benzofuro[3,2-c]pyrazole-3-carboxylate 1H NMR (400 MHz, CHLOROFORM-d) δ=7.95 (s, 1H), 7.63 (d, J=3.2 Hz, 1H), 7.60-7.57 (m, 1H), 7.55-7.51 (m, 1H), 7.23 (s, 1H), 4.56 (s, 2H), 3.99 (s, 3H), 1.48 (t, J=7.2 Hz, 3H).

### Synthesis of 7-bromo-6-methoxy-1-(3-thienyl)benzofuro[3,2-c]pyrazole-3-carboxylic acid

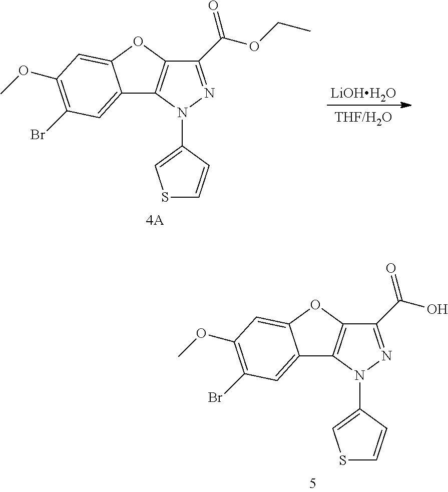

To a mixture of ethyl 7-bromo-6-methoxy-1-(3-thienyl)benzofuro[3,2-c]pyrazole-3-carboxylate (200 mg, 474.76 μmol, 1 eq) in THF (2 mL) and H₂O (1 mL) was added LiOH·H2O (59.76 mg, 1.42 mmol, 3 eq), the mixture was stirred at 25° C. for 2 h. The mixture concentrated under vacuum to remove organic phase, the resulting mixture was adjusted pH=6 by 1 N HCl, There were precipitates formed in the reaction mixture, the resulting mixture was filtered and filter cake was dried under vacuum to give 7-bromo-6-methoxy-1-(3-thienyl)benzofuro[3,2-c]pyrazole-3-carboxylic acid (160 mg, 406.91 μmol, 86% yield) as a brown solid.

### Synthesis of [7-bromo-6-methoxy-1-(3-thienyl)benzofuro[3,2-c]pyrazol-3-yl]-(3,3-dimethylmorpholin-4-yl)methanone

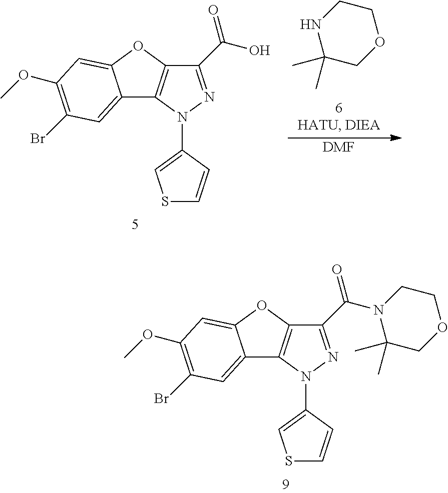

To a mixture of 7-bromo-6-methoxy-1-(3-thienyl)benzofuro[3,2-c]pyrazole-3-carboxylic acid (50 mg, 127.16 μmol, 1 eq) in DMF (1 mL) was added HATU (72.52 mg, 190.74 μmol, 1.5 eq), DIEA (32.87 mg, 254.32 μmol, 44.30 μL, 2 eq), then 3,3-dimethylmorpholine (21.97 mg, 190.74 μmol, 1.5 eq) was added, the mixture was stirred at 25° C. for 16 h. The mixture was added H₂O (5 mL) and then extracted with ethyl acetate (10 mL*3). The combined organic phases were concentrated under vacuum to give a residue. The residue was purified by flash silica gel chromatography column(petroleum ether: ethyl acetate=0˜30%) to afford [7-bromo-6-methoxy-1-(3-thienyl)benzofuro[3,2-c]pyrazol-3-yl]-(3,3-dimethylmorpholin-4-yl)methanone (40 mg, 81.57 μmol, 64% yield) was obtained as a yellow solid.

### Synthesis of 5-[3-(3,3-dimethylmorpholine-4-carbonyl)-6-methoxy-1-(3-thienyl)benzofuro[3,2-c]pyrazol-7-yl]pyridine-3-carbonitrile

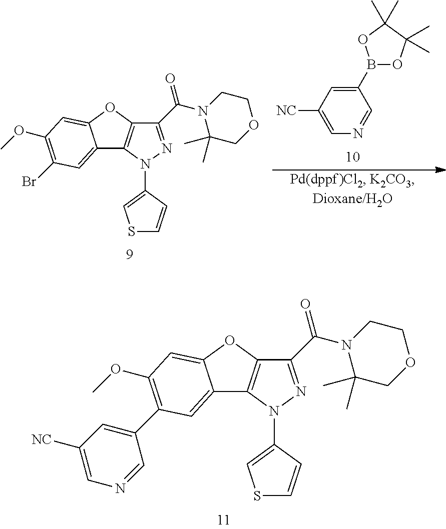 [SMILES: COc1cc2oc3c(C(=O)N4CCOCC4(C)C)nn(-c4ccsc4)c3c2cc1Br ; CC1(C)OB(c2cncc(C#N)c2)OC1(C)C ; "Pd(dppf)Cl2, K2CO3, Dioxane/H2O" ; COc1cc2oc3c(C(=O)N4CCOCC4(C)C)nn(-c4ccsc4)c3c2cc1-c1cncc(C#N)c1]

To a mixture of [7-bromo-6-methoxy-1-(3-thienyl)benzofuro[3,2-c]pyrazol-3-yl]-(3,3-dimethylmorpholin-4-yl)methanone (40 mg, 81.57 μmol, 1 eq) in H₂O (0.5 mL) and dioxane (1 mL) was added Pd(dppf)Cl₂ (5.97 mg, 8.16 y mol, 0.1 eq), K₂CO₃ (33.82 mg, 244.71 μmol, 3 eq), 5-(4,4,5,5-tetramethyl-1,3,2-dioxaborolan-2-yl)pyridine-3-carbonitrile (22.52 mg, 97.89 μmol, 1.2 eq) was added, the mixture was stirred at 80° C. for 16 h under N₂. The mixture was concentrated under vacuum to give a residue. the residue was purified by prep-TLC(petroleum ether: ethyl acetate=3:1) to afford 5-[3-(3,3-dimethylmorpholine-4-carbonyl)-6-methoxy-1-(3-thienyl)benzofuro[3,2-c]pyrazol-7-yl]pyridine-3-carbonitrile (30 mg, 58.42 μmol, 72% yield) as a brown solid.

### Synthesis of 5-[3-(3,3-dimethylmorpholine-4-carbonyl)-6-methoxy-1-(3-thienyl)benzofuro[3,2-c]pyrazol-7-yl]pyridine-3-carbonitrile and 5-[3-(3,3-dimethylmorpholine-4-carbonyl)-6-methoxy-1-(3-thienyl)benzofuro[3,2-c]pyrazol-7-yl]pyridine-3-carbonitrile

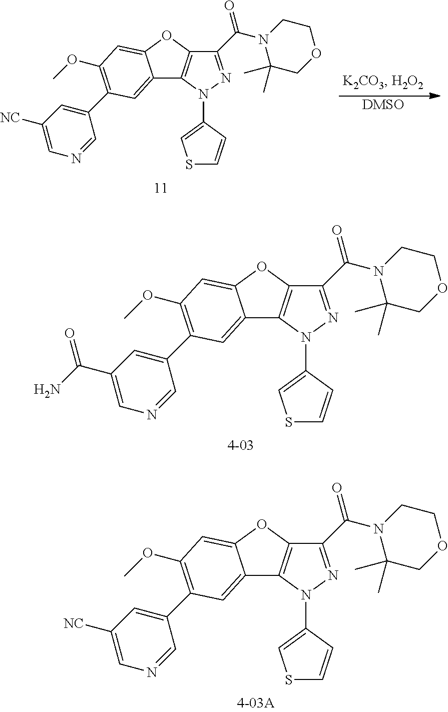

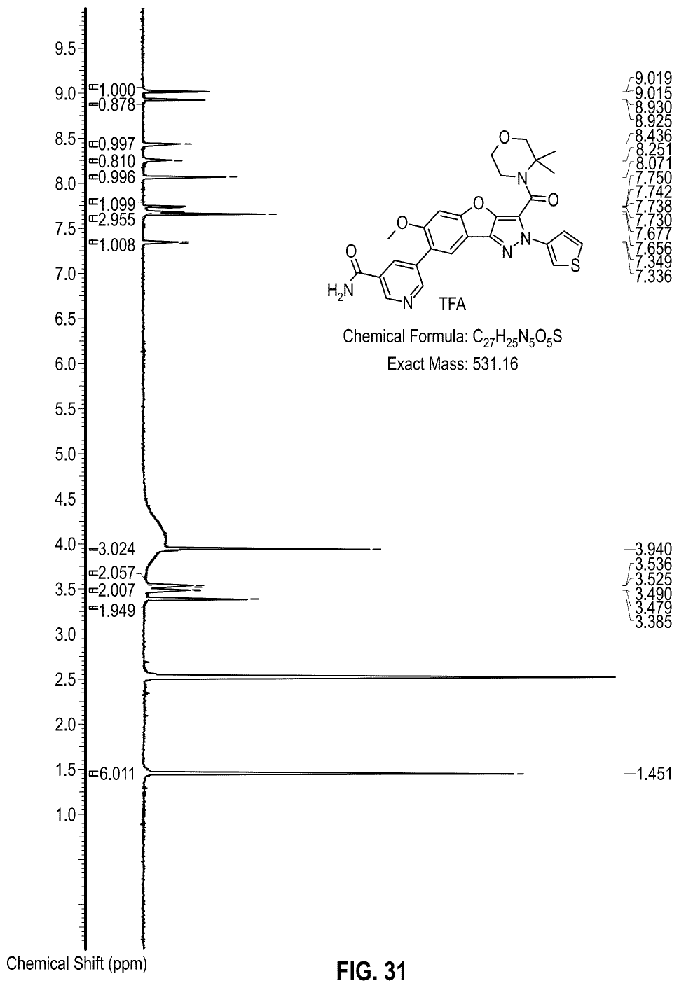

To a mixture of 5-[3-(3,3-dimethylmorpholine-4-carbonyl)-6-methoxy-1-(3-thienyl)benzofuro[3,2-c]pyrazol-7-yl]pyridine-3-carbonitrile (30 mg, 58.42 μmol, 1 eq) in DMSO (1 mL) was added H₂O₂ (66.22 mg, 584.15 μmol, 56.12 μL, 30% purity, 10 eq), K₂CO₃ (16.15 mg, 116.83 μmol, 2 eq), the mixture was stirred at 60° C. for 1 h. The mixture was added saturated Na₂SO₃ aqueous solution (20 mL). The mixture was adjusted pH=7 by 1 N HCl, the resulting mixture was detected by KI paper(did not turn blue), the resulting mixture was extracted with ethyl acetate(10 mL*3). The combined organic phases were concentrated under vacuum to give a residue. The residue was purified by prep-HPLC(column: Phenomenex Luna C18 150*25 mm*10 um;mobile phase: [water(TFA)-ACN];gradient:33%-63% B over 9 min) followed by lyophilization to afford 5-[3-(3,3-dimethylmorpholine-4-carbonyl)-6-methoxy-1-(3-thienyl)benzofuro[3,2-c]pyrazol-7-yl]pyridine-3-carboxamide (8.55 mg, 13.11 μmol, 22% yield, 99% purity, TFA) as an off-white solid. LCMS (ESI): m/z [M+H] calcd for C₂₇H₂₆N₅O₅S: 532.16; found: 532.1. 1H NMR (400 MHz, DMSO-d6) δ=9.02 (d, J=1.7 Hz, 1H), 8.89 (d, J=1.6 Hz, 1H), 8.39 (s, 1H), 8.21 (br s, 1H), 8.00 (br s, 1H), 7.90 (s, 1H), 7.83-7.77 (m, 1H), 7.69 (s, 1H), 7.67-7.62 (m, 2H), 3.91 (s, 5H), 3.82 (br d, J=4.9 Hz, 2H), 3.48 (s, 2H), 1.48 (s, 6H). FIG. 31 shows the nuclear magnetic resonance of Compound 4-03A.

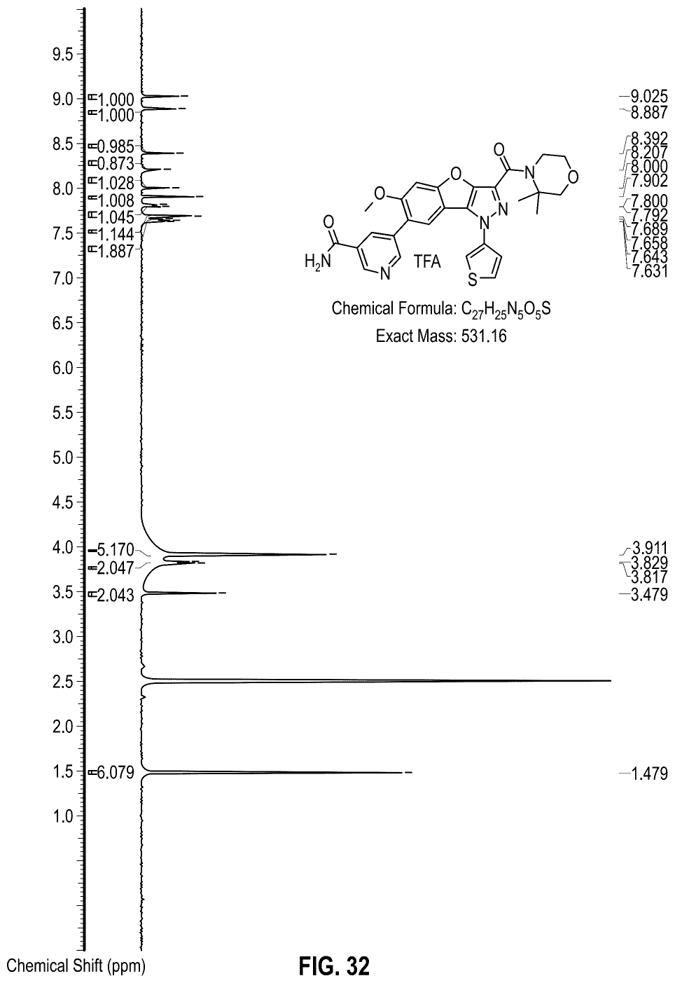

5-[3-(3,3-dimethylmorpholine-4-carbonyl)-6-methoxy-1-(3-thienyl)benzofuro[3,2-c]pyrazol-7-yl]pyridine-3-carbonitrile (9.42 mg, 14.86 μmol, 25% yield, 99% purity, TFA) was obtained as a brown solid. LCMS (ESI): m/z [M+H] calcd for C₂₇H₂₄N₅O₄S: 514.15; found: 514.2. 1H NMR (400 MHz, DMSO-d6) δ=9.10 (s, 1H), 9.06 (s, 1H), 8.61-8.56 (m, 1H), 8.05 (br s, 1H), 7.98 (s, 1H), 7.85 (br s, 1H), 7.74 (s, 1H), 7.70-7.67 (m, 1H), 3.96 (br s, 5H), 3.86 (br s, 2H), 3.52 (br s, 2H), 1.52 (s, 6H). FIG. 32 shows the nuclear magnetic resonance of Compound 4-03.

### Synthesis of Compound 4-04

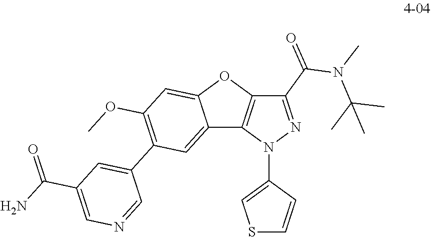 [SMILES: COc1cc2oc3c(C(=O)N(C)C(C)(C)C)nn(-c4ccsc4)c3c2cc1-c1cncc(C(N)=O)c1]

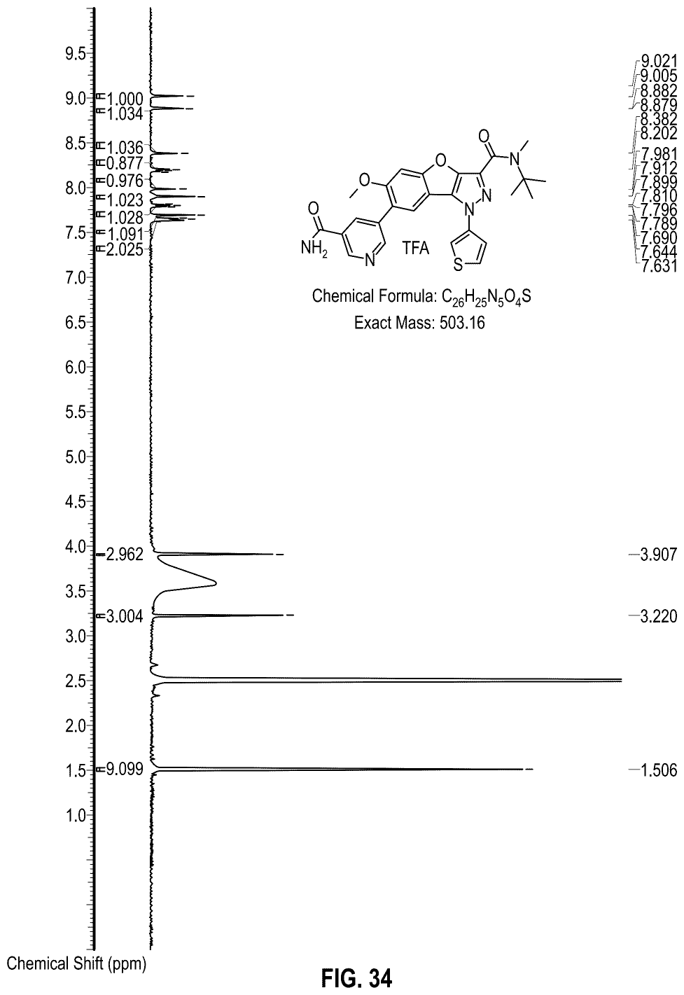

Compound 4-04 was synthesized via a similar procedure as example 4. LCMS (ESI): m/z [M+H] calcd for C₂₆H₂₆N₅O₄S: 504.16; found: 504.1 1H NMR (400 MHz, DMSO-d6) δ=9.02 (s, 1H), 8.88 (d, J=1.5 Hz, 1H), 8.38 (s, 1H), 8.23-8.17 (m, 1H), 7.98 (br s, 1H), 7.90 (s, 1H), 7.82-7.77 (m, 1H), 7.69 (s, 1H), 7.67-7.62 (m, 2H), 3.91 (s, 3H), 3.22 (s, 3H), 1.51 (s, 9H). FIG. 34 shows the nuclear magnetic resonance of Compound 4-04.

### Synthesis of Compound 4-03A

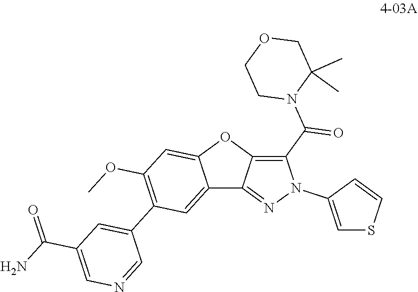 [SMILES: COc1cc2oc3c(C(=O)N4CCOCC4(C)C)n(-c4ccsc4)nc3c2cc1-c1cncc(C(N)=O)c1]

Compound 4-03A was synthesized via a similar procedure as example 4. LCMS (ESI): m/z [M+H] calcd for C₂₇H₂₆N₅O₅S 532.16; found: 532.1. 1H NMR (400 MHz, DMSO-d6) δ=9.02 (d, J=1.8 Hz, 1H), 8.93 (d, J=1.8 Hz, 1H), 8.44 (br s, 1H), 8.25 (br s, 1H), 8.07 (s, 1H), 7.74 (dd, J=3.2, 5.0 Hz, 1H), 7.70-7.64 (m, 3H), 7.34 (d, J=5.0 Hz, 1H), 3.94 (s, 3H), 3.53 (br d, J=4.4 Hz, 2H), 3.48 (br d, J=4.4 Hz, 2H), 3.39 (s, 2H), 1.45 (s, 6H). FIG. 31 shows the nuclear magnetic resonance of Compound 4-03A.

### Synthesis of Compound 4-04A

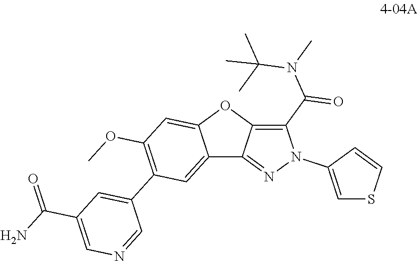 [SMILES: COc1cc2oc3c(C(=O)N(C)C(C)(C)C)n(-c4ccsc4)nc3c2cc1-c1cncc(C(N)=O)c1]

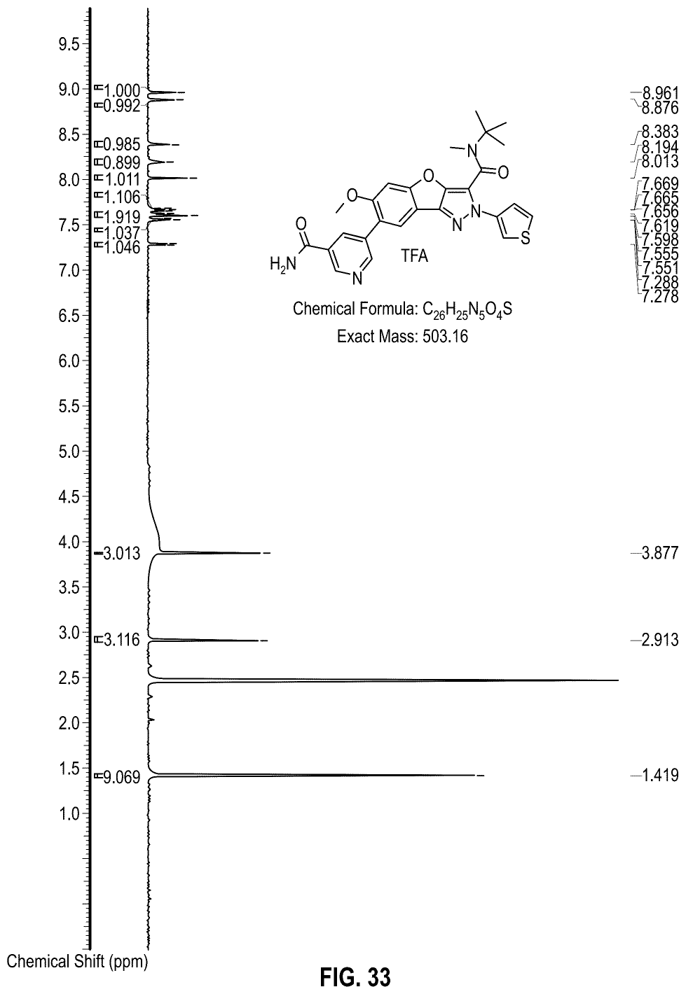

Compound 4-04A was synthesized via a similar procedure as example 4. LCMS (ESI): m/z [M+H] calcd for C₂₆H₂₆N₅O₄S: 504.16; found: 504.1. 1H NMR (400 MHz, DMSO-d6) δ=8.96 (s, 1H), 8.88 (s, 1H), 8.38 (br s, 1H), 8.19 (br s, 1H), 8.01 (s, 1H), 7.67 (dd, J=3.4, 5.0 Hz, 1H), 7.64-7.58 (m, 2H), 7.55 (br d, J=1.5 Hz, 1H), 7.28 (d, J=4.0 Hz, 1H), 3.88 (s, 3H), 2.91 (s, 3H), 1.42 (s, 9H). FIG. 33 shows the nuclear magnetic resonance of Compound 4-04A.

### Example 5

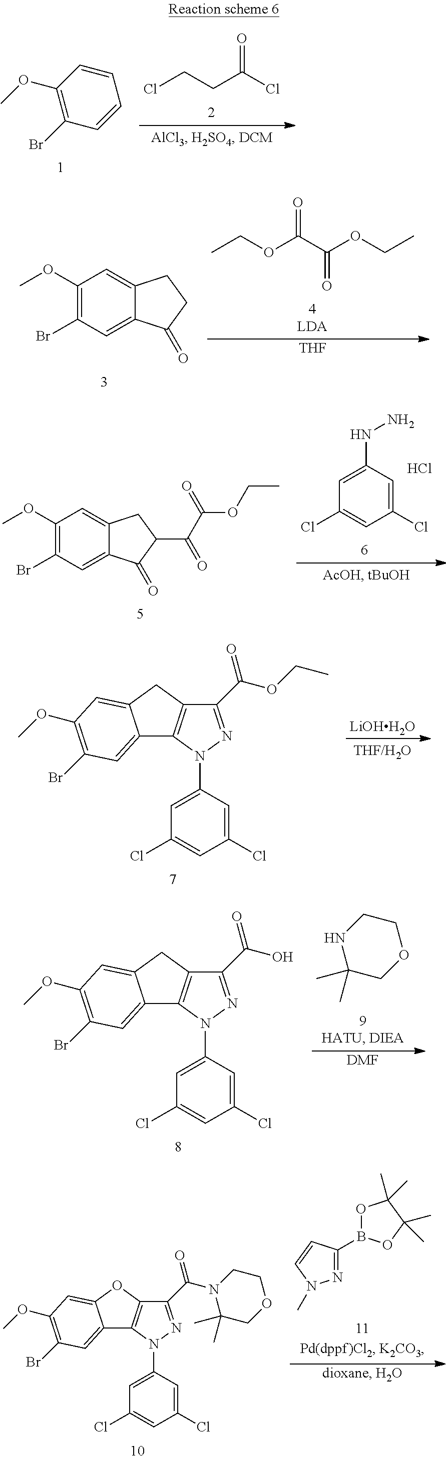

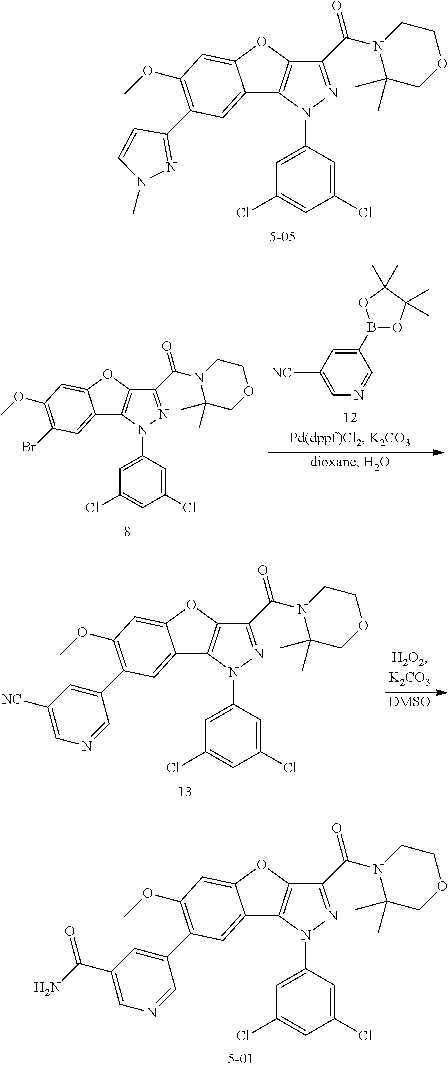

### Synthesis of 6-bromo-5-methoxy-indan-1-one

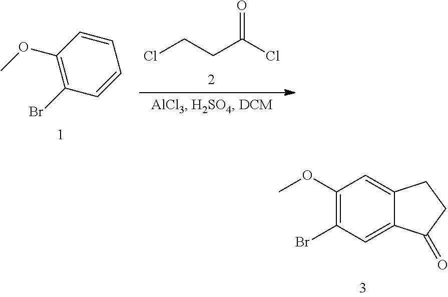

To a solution of 1-bromo-2-methoxy-benzene (2 g, 10.69 mmol, 1.33 mL, 1 eq) and 3-chloropropanoyl chloride (1.49 g, 11.76 mmol, 1.13 mL, 1.1 eq) in DCM (10 mL) was added AlCl₃ (1.57 g, 11.76 mmol, 642.80 μL, 1.1 eq) at 0° C. The mixture was stirred at 0° C. for 0.5 h, then H₂SO₄ (10 mL) was added. The mixture was then concentrated under vacuum to remove DCM. The resulting mixture was stirred at 100° C. for 2 h. The mixture was poured into water slowly and left overnight. There were precipitates formed in the reaction mixture which was filtered and the filter cake was dried in vacuo to get the crude product. The crude product was purified by flash silica gel chromatography (ISCO®; 40 g SepaFlash® Silica Flash Column, Eluent of 0˜50% Petroleum ether gradient/Ethylacetate @60 mL/min) to afford 6-bromo-5-methoxy-indan-1-one (900 mg, 3.73 mmol, 35% yield) as a white solid. ¹H NMR (400 MHz, DMSO-d₆) δ=7.77 (s, 1H), 7.30 (s, 1H), 3.96 (s, 3H), 3.08-3.00 (m, 2H), 2.66-2.59 (m, 2H).

### Synthesis of ethyl 2-(6-bromo-5-methoxy-1-oxo-indan-2-yl)-2-oxo-acetate

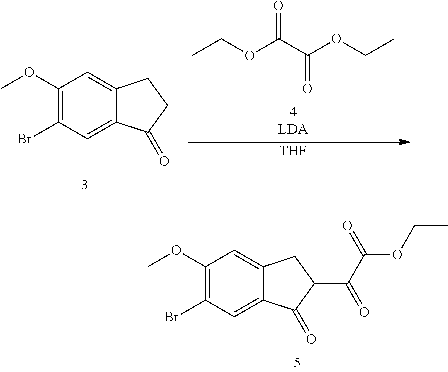

To a mixture of 6-bromo-5-methoxy-indan-1-one (400 mg, 1.66 mmol, 1 eq) in THF (6 mL) was added LDA (2 M, 1.24 mL, 1.5 eq) at −78° C. under N₂ atmosphere. The mixture was stirred at −78° C. for 15 min, then diethyl oxalate (387.96 mg, 2.65 mmol, 362.58 μL, 1.6 eq) was added. The mixture was stirred at 0° C. for 1 h. The reaction mixture was acidified to pH=6 with 1 M HCl aqueous solution, whereupon precipitates formed in the reaction mixture. It was filtered and the filter cake was dried in vacuo to afford ethyl 2-(6-bromo-5-methoxy-1-oxo-indan-2-yl)-2-oxo-acetate (470 mg, 1.38 mmol, 83% yield) as a white solid. ¹H NMR (400 MHz, DMSO-d₆) δ=8.03-7.86 (m, 1H), 7.42 (br s, 1H), 4.36-4.23 (m, 2H), 3.98 (s, 3H), 3.89-3.76 (m, 2H), 1.32 (t, J=7.07 Hz, 3H).

### Synthesis of ethyl 7-bromo-1-(3,5-dichlorophenyl)-6-methoxy-4H-indeno[1,2-c]pyrazole-3-carboxylate

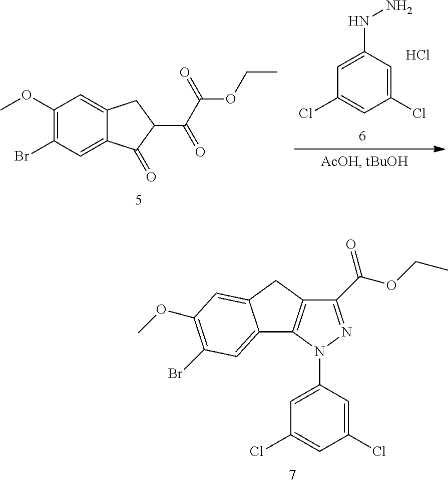

To a mixture of ethyl 2-(6-bromo-5-methoxy-1-oxo-indan-2-yl)-2-oxo-acetate (300 mg, 879.37 μmol, 1 eq) in t-BuOH (4 mL) was added AcOH (264.04 mg, 4.40 mmol, 251.71 μL, 5 eq) and (3,5-dichlorophenyl)hydrazine hydrochloride (187.74 mg, 879.37 μmol, 1 eq). The mixture was stirred at 90° C. for 1 h. The mixture was concentrated under vacuum to give a residue. The residue was triturated with ethyl acetate (5 mL) at 15° C. for 0.5 h to afford ethyl 7-bromo-1-(3,5-dichlorophenyl)-6-methoxy-4H-indeno[1,2-c]pyrazole-3-carboxylate (340 mg, 705.17 μmol, 80% yield) as a gray solid.

### Synthesis of 7-bromo-1-(3,5-dichlorophenyl)-6-methoxy-4H-indeno[1,2-c]pyrazole-3-carboxylic acid

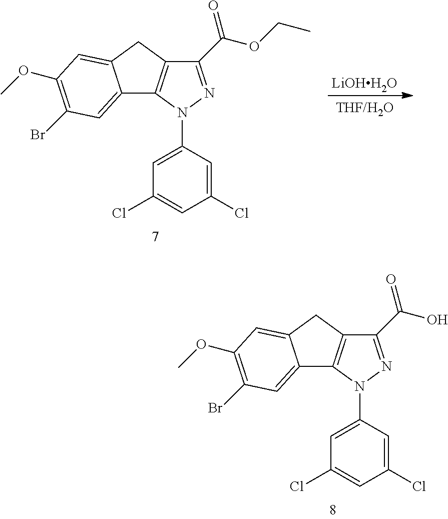

To a mixture of ethyl 7-bromo-1-(3,5-dichlorophenyl)-6-methoxy-4H-indeno[1,2-c]pyrazole-3-carboxylate (340 mg, 705.17 μmol, 1 eq) in THF (3 mL) and H2O (3 mL) was added LiOH. H₂O (88.77 mg, 2.12 mmol, 3 eq). The mixture was stirred at 15° C. for 16 h. The reaction mixture was acidified to pH=3 with 1 M HCl aqueous solution, and precipitates formed in the reaction mixture. It was filtered and the filter cake was dried over under vacuum to afford 7-bromo-1-(3, 5-dichlorophenyl)-6-methoxy-4H-indeno [1,2-c]pyrazole-3-carboxylic acid (270 mg, crude) as an off-white solid. ¹H NMR (400 MHz, DMSO-d₆) δ=7.88 (d, J=1.63 Hz, 2H), 7.84 (d, J=1.75 Hz, 1H), 7.50 (d, J=6.38 Hz, 2H), 3.92 (s, 3H), 3.77 (s, 2H).

### Synthesis of [7-bromo-1-(3,5-dichlorophenyl)-6-methoxy-4H-indeno[1,2-c]pyrazol-3-yl]-(3,3-dimethylmorpholin-4-yl)methanone

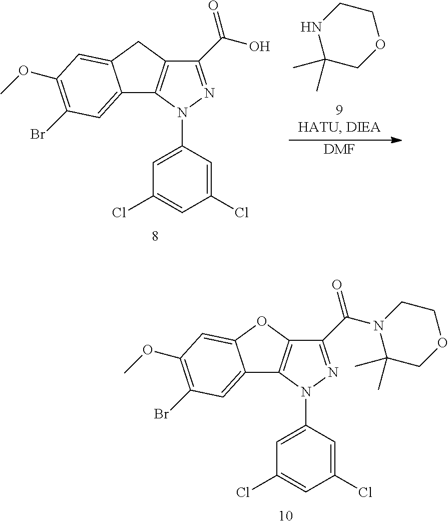

To a mixture of 7-bromo-1-(3,5-dichlorophenyl)-6-methoxy-4H-indeno[1,2-c]pyrazole-3-carboxylic acid (270 mg, 594.58 μmol, 1 eq) in DMF (4 mL) was added HATU (226.08 mg, 594.58 μmol, 1 eq) and DIEA (230.53 mg, 1.78 mmol, 310.69 μL, 3 eq). The mixture was stirred at 15° C. for 15 min, then 3,3-dimethylmorpholine (82.18 mg, 713.50 μmol, 1.2 eq) was added. The mixture was stirred at 25° C. for 16 h. The mixture was poured into H₂O (20 mL), whereby a precipitate formed in the reaction mixture. It was filtered and the filter cake was dried in vacuo to give the crude product. The crude product was triturated with 2-methoxy-2-methyl-propane (5 mL) at 15° C. for 15 min to afford [7-bromo-1-(3,5-dichlorophenyl)-6-methoxy-4H-indeno[1,2-c]pyrazol-3-yl]-(3,3-dimethylmorpholin-4-yl)methanone (150 mg, 272.10 μmol, 46% yield) as a yellow solid.

### Synthesis of [1-(3,5-dichlorophenyl)-6-methoxy-7-(1-methylpyrazol-3-yl)-4H-indeno[1,2-c]pyrazol-3-yl]-(3,3-dimethylmorpholin-4-yl)methanone

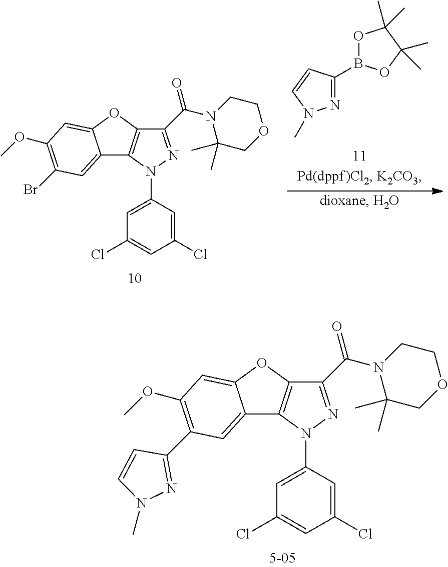

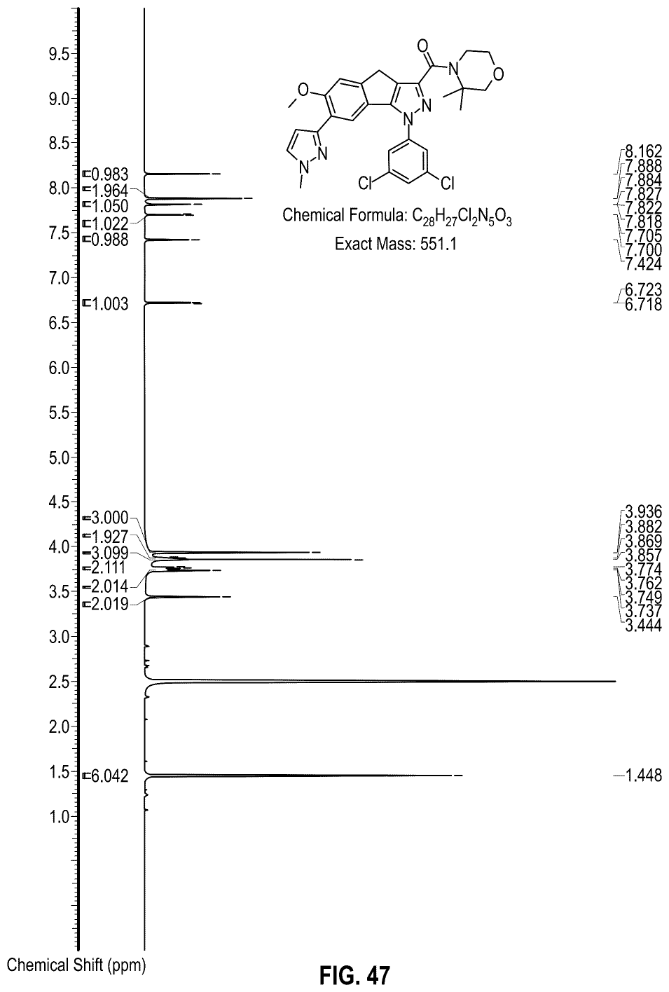

To a mixture of 1-methyl-3-(4,4,5,5-tetramethyl-1,3,2-dioxaborolan-2-yl)pyrazole (67.94 mg, 326.53 μmol, 1.2 eq) in dioxane (2 mL) and H₂O (0.4 mL) was added Pd(dppf)Cl₂ (39.82 mg, 54.42 μmol, 0.2 eq), [7-bromo-1-(3,5-dichlorophenyl)-6-methoxy-4H-indeno[1,2-c]pyrazol-3-yl]-(3,3-dimethylmorpholin-4-yl)methanone (150 mg, 272.10 μmol, 1 eq) and K₂CO₃ (112.82 mg, 816.31 μmol, 3 eq). The mixture was stirred at 80° C. for 16 h under N₂ atmosphere. The mixture was filtered and the filtrate was concentrated under vacuum to give a residue. The residue was purified by prep-HPLC (column: Phenomenex Luna C18 150*25 mm*10 um; mobile phase: [water (TFA)-ACN];gradient: 65%-95% B over 9 min) followed by lyophilization to afford [1-(3,5-dichlorophenyl)-6-methoxy-7-(1-methylpyrazol-3-yl)-4H-indeno[1,2-c]pyrazol-3-yl]-(3,3-dimethylmorpholin-4-yl)methanone (42.8 mg, 72.05 μmol, 26% yield, 93% purity) was as a yellow solid. LCMS (ESI): m/z [M+H] calcd for C₂₈H₂₈N₅O₃Cl₂: 552.15; found: 552.2. ¹H NMR (400 MHz, DMSO-d₆) δ=8.16 (s, 1H), 7.89 (d, J=1.75 Hz, 2H), 7.82 (t, J=1.81 Hz, 1H), 7.70 (d, J=2.13 Hz, 1H), 7.42 (s, 1H), 6.72 (d, J=2.13 Hz, 1H), 3.94 (s, 3H), 3.89-3.86 (m, 2H), 3.86 (s, 3H), 3.78-3.75 (m, 2H), 3.74 (s, 2H), 3.44 (s, 2H), 1.45 (s, 6H). FIG. 47 shows the nuclear magnetic resonance of Compound 5-05.

### Synthesis of N-tert-butyl-7-(5-cyano-3-pyridyl)-6-methoxy-N-methyl-1-(3-thienyl)-4H-indeno[1,2-c]pyrazole-3-carboxamide

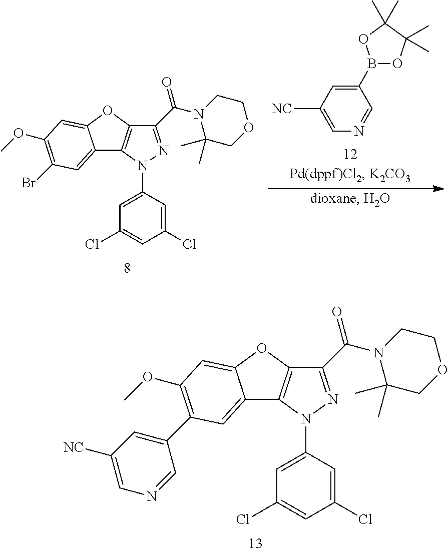 [SMILES: COc1cc2oc3c(C(=O)N4CCOCC4(C)C)nn(-c4cc(Cl)cc(Cl)c4)c3c2cc1Br ; CC1(C)OB(c2cncc(C#N)c2)OC1(C)C ; "dioxane, H2O" ; COc1cc2oc3c(C(=O)N4CCOCC4(C)C)nn(-c4cc(Cl)cc(Cl)c4)c3c2cc1-c1cncc(C#N)c1]

To a solution of 5-(4,4,5,5-tetramethyl-1,3,2-dioxaborolan-2-yl)pyridine-3-carbonitrile (179.90 mg, 781.95 μmol, 1.2 eq) in dioxane (2.5 mL) and H₂O (0.5 mL) was added K₂CO₃ (270.18 mg, 1.95 mmol, 3 eq), 7-bromo-N-tert-butyl-6-methoxy-N-methyl-1-(3-thienyl)-4H-indeno[1,2-c]pyrazole-3-carboxamide (300 mg, 651.63 μmol, 1 eq) and Pd(dppf)Cl₂ (95.36 mg, 130.33 μmol, 0.2 eq). The mixture was stirred at 80° C. for 16 h under N₂ atmosphere. The reaction mixture was poured into H₂O (5 mL) and ethyl acetate (5 mL), then the mixture was separated. The aqueous phase was extracted with ethyl acetate (5 mL*3). The combined organic phases were dried over anhydrous Na₂SO₄ and filtered. The filtrate was concentrated under vacuum to give a residue that was purified by flash silica gel chromatography (ISCO®; 4 g SepaFlash® Silica Flash Column, Eluent of 0-70% Petroleum ether gradient/Ethyl acetate @30 mL/min) to afford N-tert-butyl-7-(5-cyano-3-pyridyl)-6-methoxy-N-methyl-1-(3-thienyl)-4H-indeno[1,2-c]pyrazole-3-carboxamide (200 mg, 413.58 μmol, 63% yield) as a brown solid.

### Synthesis of 5-[1-(3,5-dichlorophenyl)-3-(3,3-dimethylmorpholine-4-carbonyl)-6-methoxy-4H-indeno[1,2-c]pyrazol-7-yl]pyridine-3-carboxamide

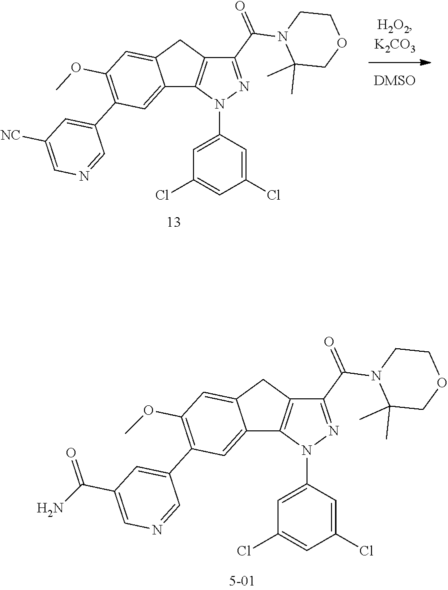 [SMILES: COc1cc2c(cc1-c1cncc(C#N)c1)-c1c(c(C(=O)N3CCOCC3(C)C)nn1-c1cc(Cl)cc(Cl)c1)C2 ; COc1cc2c(cc1-c1cncc(C(N)=O)c1)-c1c(c(C(=O)N3CCOCC3(C)C)nn1-c1cc(Cl)cc(Cl)c1)C2]

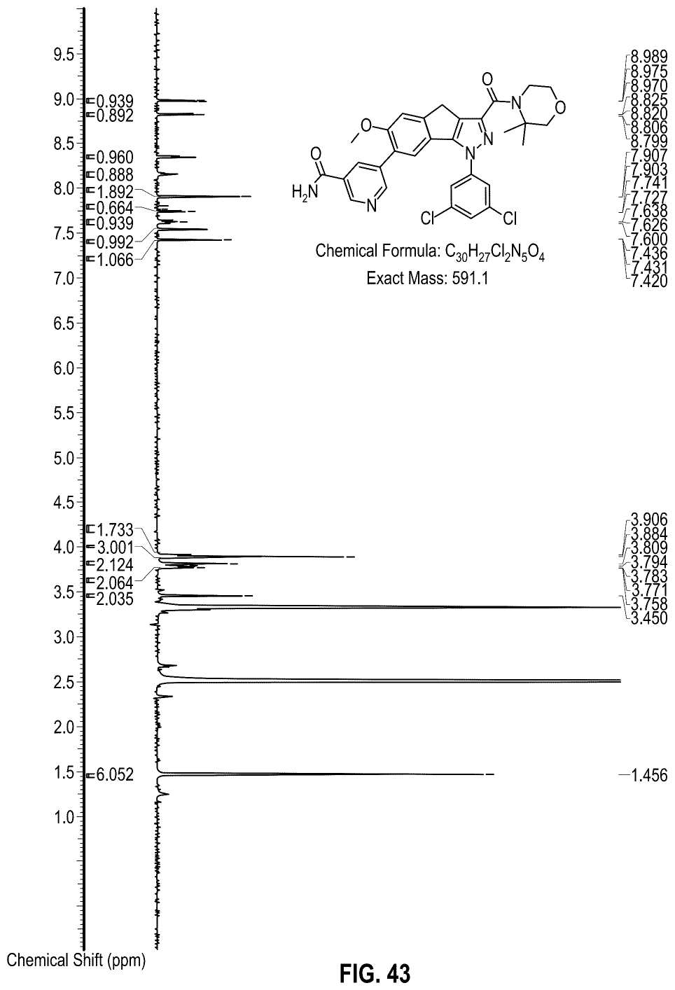

To a mixture of [1-(3,5-dichlorophenyl)-7-(5-cyano-3-pyridyl)-6-methoxy-4H-indeno[1,2-c]pyrazol-3-yl]-(3,3-dimethylmorpholin-4-yl)methanone (70 mg, 121.85 μmol, 1 eq) in DMSO (2 mL) was added H₂O₂ (290 mg, 2.56 mmol, 245.76 μL, 30% purity, 20.99 eq) and K₂CO₃ (33.68 mg, 243.71 μmol, 2 eq). The mixture was stirred at 15° C. for 15 min. The mixture was poured into saturated aqueous Na₂S₂O₃ (10 mL), and the whole stirred at 15° C. for another 1 hour. The resulting mixture was extracted with ethyl acetate (10 mL*3) and the combined organic phases were concentrated under vacuum to give a residue. The residue was purified by prep-HPLC (column: Waters Xbridge 150*25 mm*5 um; mobile phase: [water (NH₄HCO₃)-ACN]; gradient: 40%-70% B over 9 min) followed by lyohilization to afford 5-[1-(3,5-dichlorophenyl)-3-(3,3-dimethylmorpholine-4-carbonyl)-6-methoxy-4H-indeno[1,2-c]pyrazol-7-yl]pyridine-3-carboxamide (5.33 mg, 8.82 μmol, 7% yield, 98% purity) as an off-white solid. LCMS (ESI): m/z [M+H] calcd for C₃₀H₂₈N₅O₄Cl₂: 592.14; found: 592.2. ¹H NMR (400 MHz, DMSO-d₆) δ=8.97 (d, J=1.96 Hz, 1H), 8.82 (d, J=2.08 Hz, 1H), 8.37-8.32 (m, 1H), 8.18-8.12 (m, 1H), 7.90 (d, J=1.71 Hz, 2H), 7.77-7.72 (m, 1H), 7.65-7.59 (m, 1H), 7.57-7.53 (m, 1H), 7.44-7.39 (m, 1H), 3.91 (br s, 2H), 3.88 (s, 3H), 3.84-3.79 (m, 2H), 3.79-3.74 (m, 2H), 3.45 (s, 2H), 1.46 (s, 6H). FIG. 43 shows the nuclear magnetic resonance of Compound 5-01.

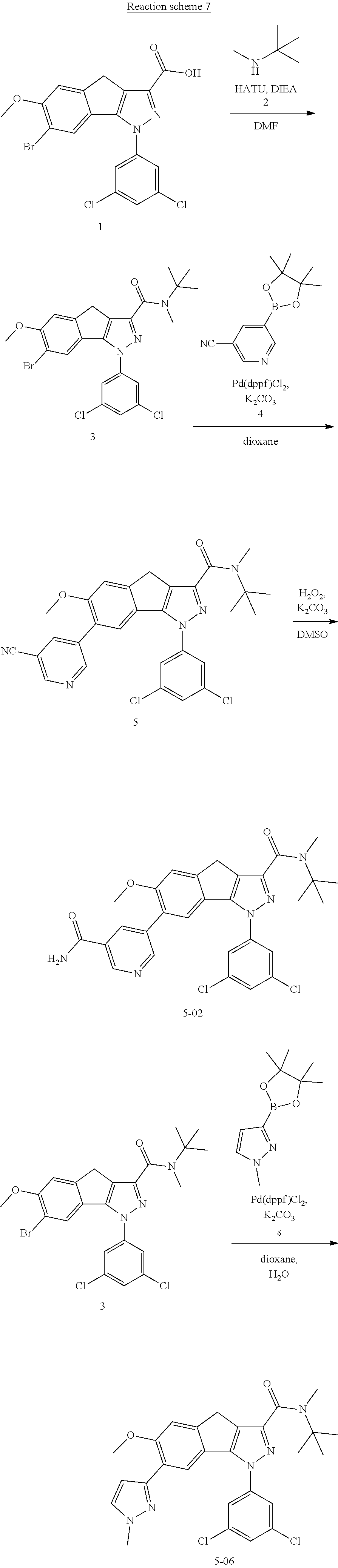

### Synthesis of 7-bromo-N-tert-butyl-1-(3,5-dichlorophenyl)-6-methoxy-N-methyl-4H-indeno[1,2-c]pyrazole-3-carboxamide

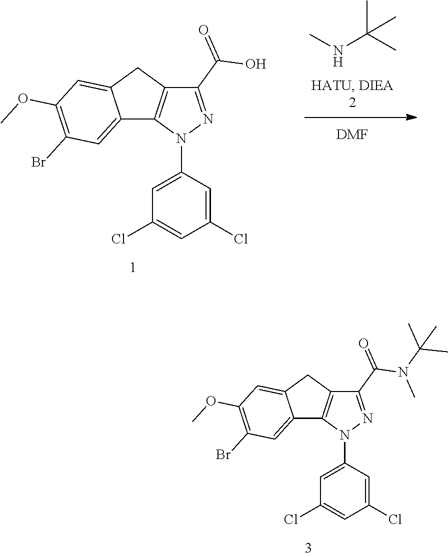

To a mixture of 7-bromo-1-(3,5-dichlorophenyl)-6-methoxy-4H-indeno[1,2-c]pyrazole-3-carboxylic acid (500 mg, 1.10 mmol, 1 eq) in DMF (5 mL) was added HATU (502.39 mg, 1.32 mmol, 1.2 eq) and DIEA (426.92 mg, 3.30 mmol, 575.36 μL, 3 eq). The mixture was stirred at 15° C. for 15 min, then N-2-dimethylpropan-2-amine (143.96 mg, 1.65 mmol, 198.02 μL, 1.5 eq) was added in the mixture. The mixture was stirred at 15° C. for 16 h then poured into H₂O (20 mL), whereby precipitation was observed. The precipitate was collected by filtration and the filter cake was dried in vacuo to afford 7-bromo-N-tert-butyl-1-(3,5-dichlorophenyl)-6-methoxy-N-methyl-4H-indeno[1,2-c]pyrazole-3-carboxamide (520 mg, 993.79 μmol, 90% yield) as an off-white solid.

### Synthesis of N-tert-butyl-1-(3,5-dichlorophenyl)-7-(5-cyano-3-pyridyl)-6-methoxy-N-methyl-4H-indeno[1,2-c]pyrazole-3-carboxamide

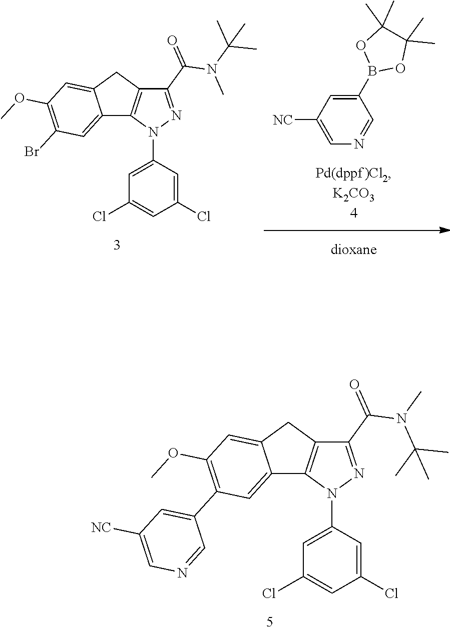 [SMILES: COc1cc2c(cc1Br)-c1c(c(C(=O)N(C)C(C)(C)C)nn1-c1cc(Cl)cc(Cl)c1)C2 ; CC1(C)OB(c2cncc(C#N)c2)OC1(C)C ; COc1cc2c(cc1-c1cncc(C#N)c1)-c1c(c(C(=O)N(C)C(C)(C)C)nn1-c1cc(Cl)cc(Cl)c1)C2]

To a mixture of 5-(4,4,5,5-tetramethyl-1,3,2-dioxaborolan-2-yl) pyridine-3-carbonitrile (158.29 mg, 688.01 μmol, 1.2 eq) in dioxane (2.5 mL) and H₂O (0.5 mL) was added K₂CO₃ (237.72 mg, 1.72 mmol, 3 eq), 7-bromo-N-tert-butyl-1-(3,5-dichlorophenyl)-6-methoxy-N-methyl-4H-indeno[1,2-c]pyrazole-3-carboxamide (300 mg, 573.34 μmol, 1 eq) and Pd(dppf)Cl₂ (83.90 mg, 114.67 μmol, 0.2 eq). The mixture was stirred at 80° C. for 16 h under N₂ atmosphere. The reaction mixture was poured into H₂O (5 mL) and ethyl acetate (5 mL), then the mixture was separated. The aqueous phase was extracted with ethyl acetate (5 mL*3). The combined organic phases were dried over anhydrous Na₂SO₄, filtered and the filtrate was concentrated in vacuum to give a residue. The residue was purified by flash silica gel chromatography (ISCO®; 4 g SepaFlash® Silica Flash Column, Eluent of 0˜70% Petroleum ether gradient/Ethyl acetate @30 mL/min) to afford N-tert-butyl-1-(3,5-dichlorophenyl)-7-(5-cyano-3-pyridyl)-6-methoxy-N-methyl-4H-indeno[1,2-c]pyrazole-3-carboxamide (140 mg, 256.20 μmol, 45% yield) as a brown solid. ¹H NMR (400 MHz, DMSO-d₆) δ=9.00 (dd, J=12.16, 1.90 Hz, 2H), 8.45 (t, J=2.02 Hz, 1H), 7.89 (d, J=1.71 Hz, 2H), 7.74 (t, J=1.71 Hz, 1H), 7.52 (s, 1H), 7.43 (s, 1H), 3.89 (s, 3H), 3.78 (s, 2H), 3.19 (s, 3H), 1.48 (s, 9H).

### Synthesis of N-tert-butyl-7-(5-carbamoyl-3-pyridyl)-1-(3,5-dichlorophenyl)-6-methoxy-N-methyl-4H-indeno[1,2-c]pyrazole-3-carboxamide

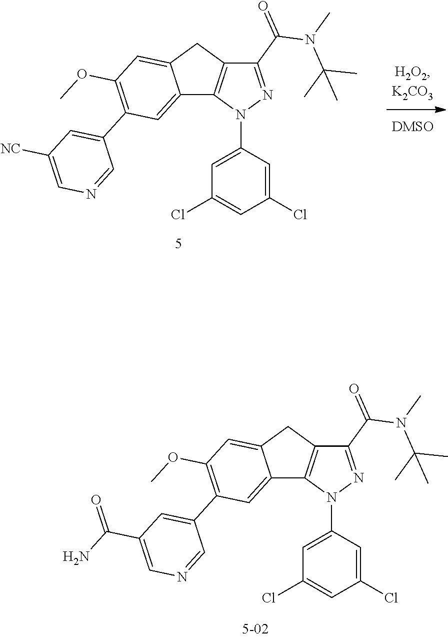 [SMILES: COc1cc2c(cc1-c1cncc(C#N)c1)-c1c(c(C(=O)N(C)C(C)(C)C)nn1-c1cc(Cl)cc(Cl)c1)C2 ; COc1cc2c(cc1-c1cncc(C(N)=O)c1)-c1c(c(C(=O)N(C)C(C)(C)C)nn1-c1cc(Cl)cc(Cl)c1)C2]

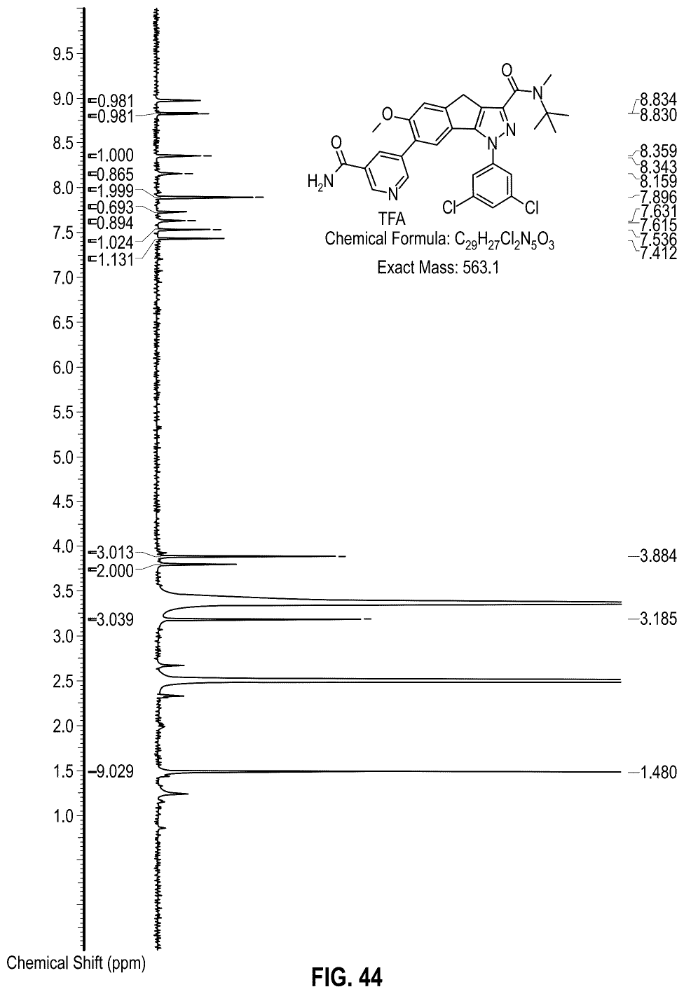

To a mixture of N-tert-butyl-1-(3,5-dichlorophenyl)-7-(5-cyano-3-pyridyl)-6-methoxy-N-methyl-4H-indeno[1,2-c]pyrazole-3-carboxamide (90 mg, 164.70 μmol, 1 eq) in DMSO (2 mL) was added H₂O₂ (290 mg, 2.56 mmol, 245.76 μL, 30% purity, 15.53 eq) and K₂CO₃ (45.53 mg, 329.40 μmol, 2 eq). The mixture was stirred at 15° C. for 15 min. The mixture was poured into saturated aqueous Na₂S₂O₃ (10 mL), and the mixture was stirred at 15° C. for another 1 hour and then extracted with ethyl acetate (10 mL*3), the combined organic phases were concentrated under vacuum to give a residue. The residue was purified by prep-HPLC (column: Phenomenex Luna C18 150*25 mm*10 um;mobile phase: [water (TFA)-ACN]; gradient: 42%-72% B) followed by lyophilization to afford N-tert-butyl-7-(5-carbamoyl-3-pyridyl)-1-(3,5-dichlorophenyl)-6-methoxy-N-methyl-4H-indeno[1,2-c]pyrazole-3-carboxamide (2.66 mg, 3.80 μmol, 2% yield, 97% purity, TFA) as an off-white solid. LCMS (ESI): m/z [M+H] calcd for C₃₁H₂₈N₅Cl₂O₅F₃: 564.15; found: 564.2. 1H NMR (400 MHz, DMSO-d6) δ=8.99-8.96 (m, 1H), 8.83 (d, J=1.59 Hz, 1H), 8.36 (s, 1H), 8.16 (s, 1H), 7.90 (s, 2H), 7.71-7.75 (m, 1H), 7.60-7.65 (m, 1H) 7.54 (s, 1H), 7.41 (s, 1H), 3.88 (s, 3H), 3.78-3.81 (m, 2H), 3.19 (s, 3H) 1.48 (s, 9H). FIG. 44 shows the nuclear magnetic resonance of Compound 5-02.

### Synthesis of N-tert-butyl-1-(3,5-dichlorophenyl)-6-methoxy-N-methyl-7-(1-methylpyrazol-3-yl)-4H-indeno[1,2-c]pyrazole-3-carboxamide

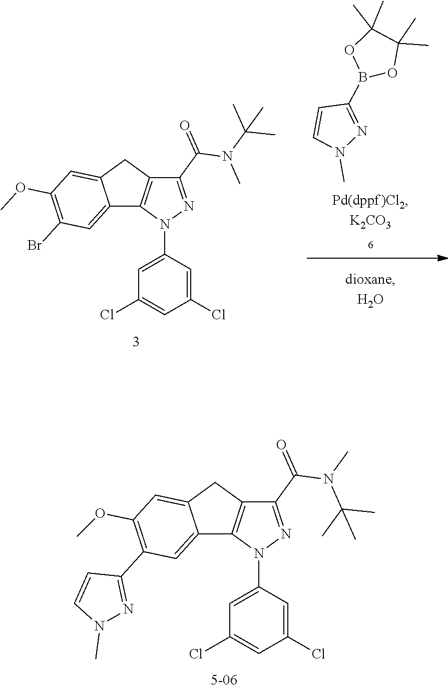

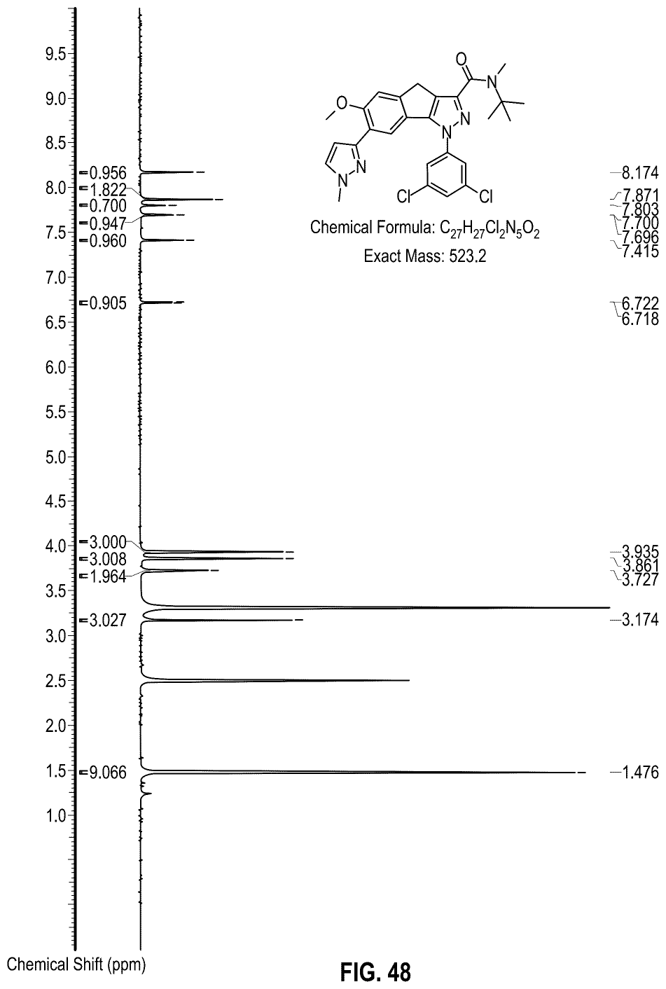

To a mixture of 1-methyl-3-(4,4,5,5-tetramethyl-1,3,2-dioxaborolan-2-yl)pyrazole (104.98 mg, 504.54 μmol, 1.2 eq) in dioxane (2.5 mL) and H₂O (0.5 mL) was added K₂CO₃ (174.33 mg, 1.26 mmol, 3 eq), 7-bromo-N-tert-butyl-1-(3,5-dichlorophenyl)-6-methoxy-N-methyl-4H-indeno[1,2-c]pyrazole-3-carboxamide (220 mg, 420.45 μmol, 1 eq) and Pd(dppf)Cl₂ (61.53 mg, 84.09 μmol, 0.2 eq). The mixture was stirred at 80° C. for 16 h under N₂ atmosphere. The mixture was filtered and the filtrate was concentrated in vacuo to give a residue which was purified by prep-HPLC (column: Waters Xbridge 150*25 mm*5 um; mobile phase: [water (NH₄HCO₃)-ACN]; gradient: 70%-100% B over 9 min) followed by lyophilization to afford N-tert-butyl-1-(3,5-dichlorophenyl)-6-methoxy-N-methyl-7-(1-methylpyrazol-3-yl)-4H-indeno[1,2-c]pyrazole-3-carboxamide (13.19 mg, 24.14 μmol, 5% yield, 96% purity) as an orange solid. LCMS (ESI): m/z [M+H] calcd for C₂₇H₂₈N₅Cl₂O₂:524.15; found: 524.2. 1H NMR (400 MHz, DMSO-d₆) δ=8.17 (s, 1H), 7.87 (s, 2H), 7.80 (s, 1H), 7.70 (d, J=1.47 Hz, 1H), 7.41 (s, 1H), 6.72 (d, J=1.83 Hz, 1H), 3.94 (s, 3H), 3.86 (s, 3H), 3.73 (s, 2H), 3.17 (s, 3H), 1.48 (s, 9H). FIG. 48 shows the nuclear magnetic resonance of Compound 5-06.

### Example 6

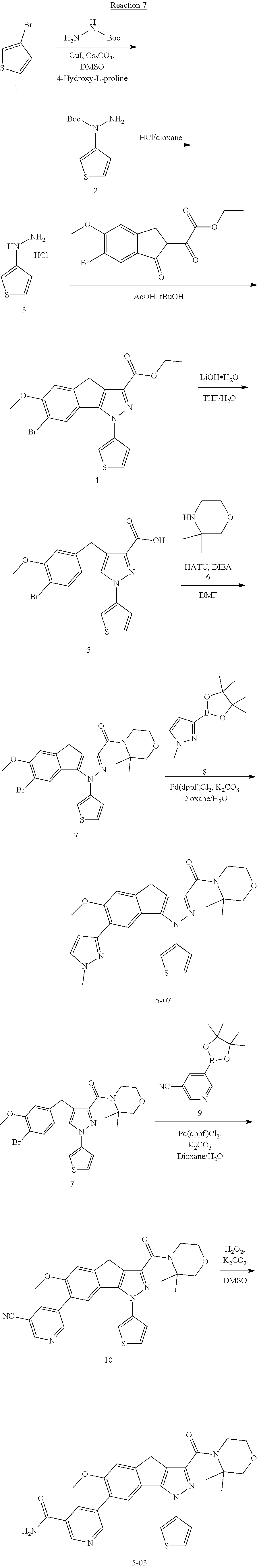

### Synthesis of tert-butyl N-amino-N-(3-thienyl)carbamate (2)

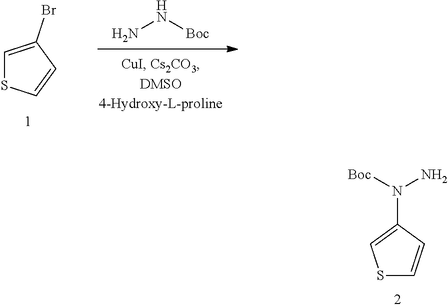 [SMILES: Brc1ccsc1 ; CC(C)(C)OC(=O)NN ; CC(C)(C)OC(=O)N(N)c1ccsc1]

A mixture of 3-bromothiophene (20 g, 122.67 mmol, 11.49 mL, 1 eq), tert-butyl N-aminocarbamate (32.42 g, 245.35 mmol, 2 eq), Cs2CO3 (79.94 g, 245.35 mmol, 2 eq),(2S,4S)-4-hydroxypyrrolidine-2-carboxylic acid (3.22 g, 24.53 mmol, 0.2 eq) and CuI (2.34 g, 12.27 mmol, 0.1 eq) in DMSO (200 mL) was degassed and purged with N₂ 3 times and then heated at 80° C. for 16 hours under N₂. The resulting residue was poured into ice-water (500 mL) and stirred for 10 min. The aqueous phase was extracted with ethyl acetate (200 mL*3) and the combined organic phases were washed with brine (200 mL*3), dried with anhydrous Na2SO4, It was filtered and the filtrate was concentrated in vacuo to provide a residue that was purified by flash silica gel chromatography (ISCO®; 330 g SepaFlash® Silica Flash Column, Eluent of 0˜20% Ethyl acetate/Petroleum ether gradient @80 mL/min). Compound tert-butyl N-amino-N-(3-thienyl)carbamate (7 g, 32.67 mmol, 27% yield) was obtained as a brown oil. 1H NMR (400 MHz, CHLOROFORM-d) δ=7.36 (br d, J=4.3 Hz, 1H), 7.18 (dd, J=3.4, 5.3 Hz, 1H), 7.14 (br s, 1H), 4.58-4.17 (m, 2H), 1.56 (s, 9H).

### Synthesis of 3-thienylhydrazine

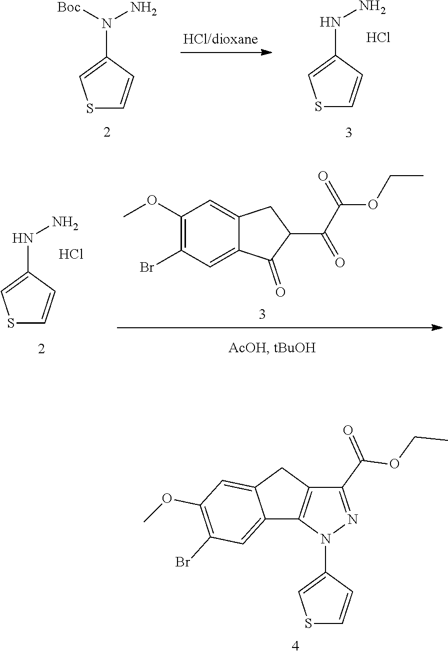

A mixture of tert-butyl N-amino-N-(3-thienyl)carbamate (1 g, 3.99 mmol, 1 eq, HCl) in EtOAc (6 mL) and HCl/EtOAc (6 mL) was stirred at 15° C. for 1 h. The mixture was concentrated under vacuum to afford 3-thienylhydrazine (600 mg, crude, HCl) as a gray solid that was used in the next without further purification.

To a mixture of 3-thienylhydrazine (600 mg, 3.98 mmol, 6.79e-1 eq, HCl) in t-BuOH (20 mL) was added AcOH (1.76 g, 29.31 mmol, 1.68 mL, 5 eq) and ethyl 2-(6-bromo-5-methoxy-1-oxo-indan-2-yl)-2-oxo-acetate (2 g, 5.86 mmol, 1 eq) and the mixture stirred at 90° C. for 16 h then concentrated in vacuo to give the crude product. The crude product was triturated with EtOH (4 mL) at 15° C. for 20 min for the 1^{st} time, then triturated with Petroleum ether: Ethyl acetate (3:1, 5 mL) at 15° C. for 20 min for the 2^{nd} time to afford ethyl 7-bromo-6-methoxy-1-(3-thienyl)-4H-indeno[1,2-c]pyrazole-3-carboxylate (1.7 g, 4.05 mmol, 69% yield) as a black solid. ¹H NMR (400 MHz, DMSO-d₆) δ=8.04 (dd, J=3.12, 1.41 Hz, 1H), 7.88-7.84 (m, 1H), 7.54 (s, 1H), 7.53-7.48 (m, 2H), 4.36-4.29 (m, 2H), 3.91 (s, 3H), 3.78 (s, 2H), 1.34 (t, J=7.15 Hz, 3H).

### Synthesis of 7-bromo-6-methoxy-1-(3-thienyl)-4H-indeno[1,2-c]pyrazole-3-carboxylic acid

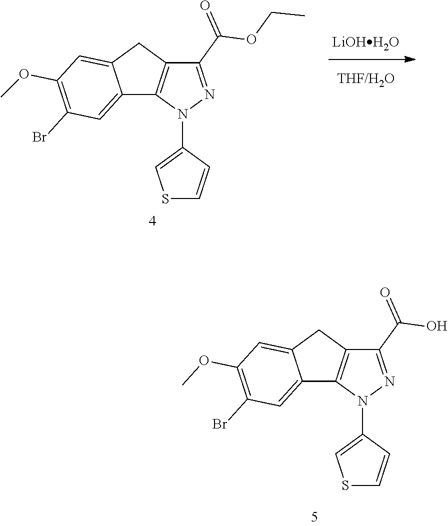

To a mixture of ethyl 7-bromo-6-methoxy-1-(3-thienyl)-4H-indeno[1,2-c]pyrazole-3-carboxylate (1.7 g, 4.05 mmol, 1 eq) in H₂O (8 mL) and THF (8 mL) was added LiOH. H₂O (510.42 mg, 12.16 mmol, 3 eq). The mixture was stirred at 15° C. for 16 h. The reaction mixture was acidified to pH=6 with 1 M HCl aqueous solution, there were precipitates formed in the reaction mixture that were collected by filtration and the filter cake was dried in vacuo to afford 7-bromo-6-methoxy-1-(3-thienyl)-4H-indeno[1,2-c]pyrazole-3-carboxylic acid (1.3 g, 3.32 mmol, 82% yield) as a brown solid.

### Synthesis of [7-bromo-6-methoxy-1-(3-thienyl)-4H-indeno[1,2-c]pyrazol-3-yl]-(3,3-dimethylmorpholin-4-yl)methanone

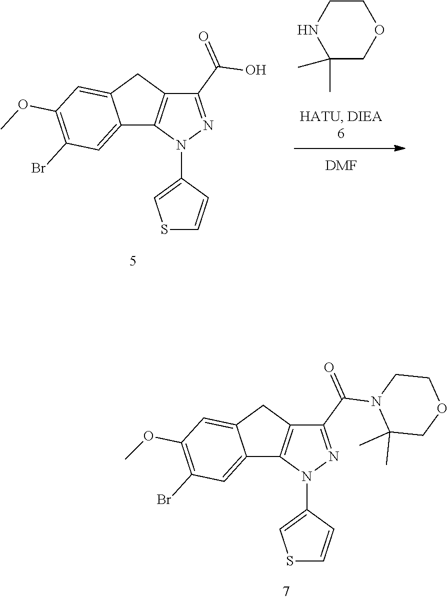

To a mixture of 7-bromo-6-methoxy-1-(3-thienyl)-4H-indeno[1,2-c]pyrazole-3-carboxylic acid (500 mg, 1.28 mmol, 1 eq) in DMF (5 mL) was added HATU (583.12 mg, 1.53 mmol, 1.2 eq) and DIEA (495.51 mg, 3.83 mmol, 667.81 μL, 3 eq). The mixture was stirred at 15° C. for 15 min, then 3,3-dimethylmorpholine (220.79 mg, 1.92 mmol, 1.5 eq) was added. The resulting mixture was stirred at 15° C. for 16 h then poured into H₂O (20 mL), there were precipitates formed in the reaction mixture. The mixture was filtered and the filter cake was collected and dried in vacuo to afford [7-bromo-6-methoxy-1-(3-thienyl)-4H-indeno[1,2-c]pyrazol-3-yl]-(3,3-dimethylmorpholin-4-yl)methanone (520 mg, 1.06 mmol, 83% yield) as a black solid.

### Synthesis of (3,3-dimethylmorpholin-4-yl)-[6-methoxy-7-(1-methylpyrazol-3-yl)-1-(3-thienyl)-4H-indeno[1,2-c]pyrazol-3-yl]methanone

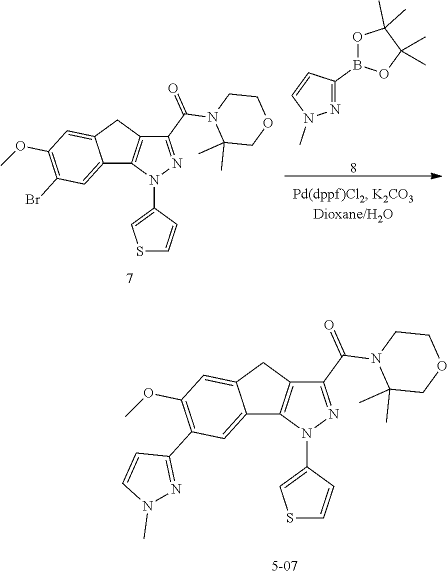

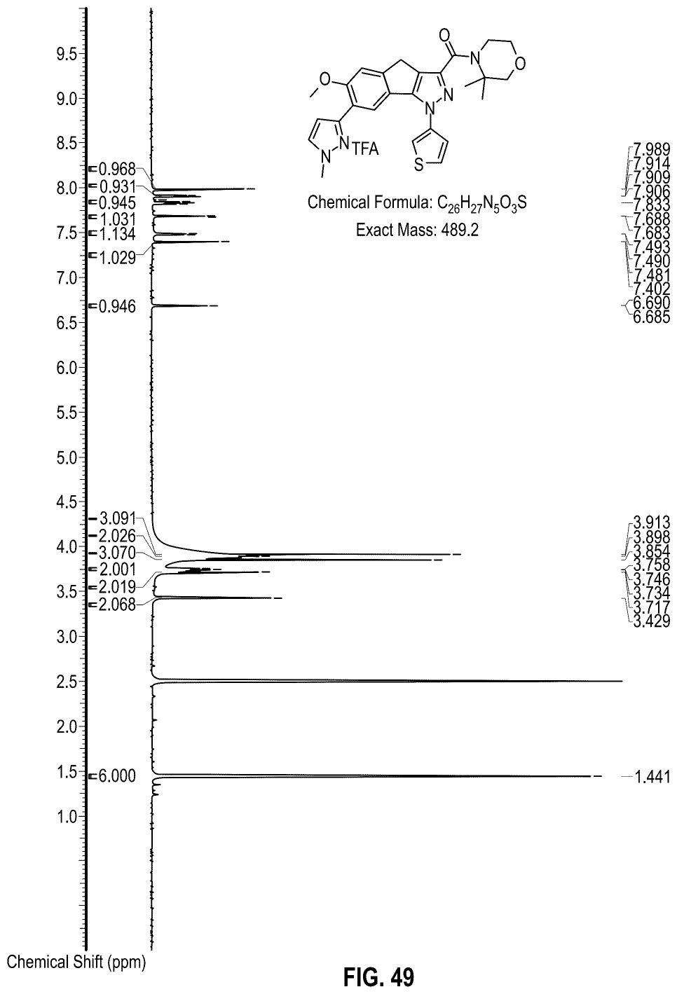

To a mixture of 1-methyl-3-(4,4,5,5-tetramethyl-1,3,2-dioxaborolan-2-yl)pyrazole (112.47 mg, 540.54 μmol, 1.2 eq) in dioxane (2.5 mL) and H₂O (0.5 mL) was added K₂CO₃ (186.77 mg, 1.35 mmol, 3 eq), [7-bromo-6-methoxy-1-(3-thienyl)-4H-indeno[1,2-c]pyrazol-3-yl]-(3,3-dimethylmorpholin-4-yl)methanone (220 mg, 450.45 μmol, 1 eq) and Pd(dppf)Cl₂ (65.92 mg, 90.09 μmol, 0.2 eq). The mixture was stirred at 80° C. for 16 h under N₂. The mixture was filtered and the filtrate was concentrated in vacuo to provide a residue that was purified by prep-HPLC (column: Phenomenex Luna C18 150*25 mm*10 um; mobile phase: [water (TFA)-ACN]; gradient: 45%-75% B) followed by lyophilization to afford (3,3-dimethylmorpholin-4-yl)-[6-methoxy-7-(1-methylpyrazol-3-yl)-1-(3-thienyl)-4H-indeno[1,2-c]pyrazol-3-yl]methanone (26.4 mg, 39.36 μmol, 9% yield, 90% purity, TFA) as an orange solid. LCMS(ESI): m/z [M+H] calcd for C₂₈H₂₈N₅O₅SF₃: 490.18; found: 490.2. ¹H NMR (400 MHz, DMSO-d₆) δ=7.99 (s, 1H), 7.69 (d, J=2.08 Hz, 1H), 7.52-7.47 (m, 1H), 7.91 (dd, J=3.06, 1.10 Hz, 1H), 7.87-7.82 (m, 1H), 7.40 (s, 1H), 6.69 (d, J=2.08 Hz, 1H), 3.91 (s, 3H), 3.89 (br s, 2H), 3.85 (s, 3H), 3.76-3.73 (m, 2H), 3.72 (s, 2H), 3.43 (s, 2H), 1.44 (s, 6H). FIG. 49 shows the nuclear magnetic resonance of Compound 5-07.

### Synthesis of (3,3-dimethylmorpholin-4-yl)-[7-(5-cyano-3-pyridyl)-6-methoxy-1-(3-thienyl)-4H-indeno[1,2-c]pyrazol-3-yl]methanone

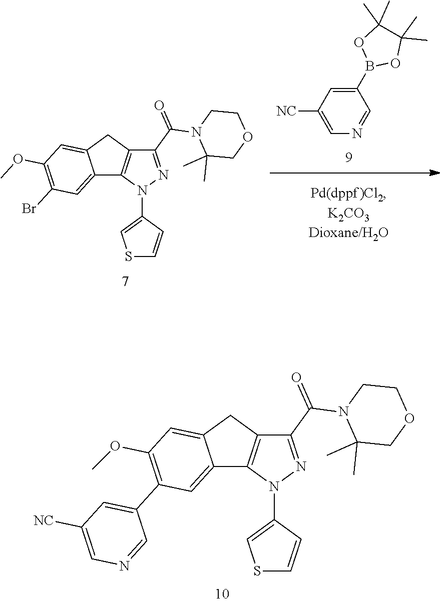 [SMILES: COc1cc2c(cc1Br)-c1c(c(C(=O)N3CCOCC3(C)C)nn1-c1ccsc1)C2 ; CC1(C)OB(c2cncc(C#N)c2)OC1(C)C ; COc1cc2c(cc1-c1cncc(C#N)c1)-c1c(c(C(=O)N3CCOCC3(C)C)nn1-c1ccsc1)C2]

To a mixture of 5-(4,4,5,5-tetramethyl-1,3,2-dioxaborolan-2-yl)pyridine-3-carbonitrile (169.59 mg, 737.11 μmol, 1.2 eq) in dioxane (2.5 mL) and H₂O (0.5 mL) was added K₂CO₃ (254.68 mg, 1.84 mmol, 3 eq), [7-bromo-6-methoxy-1-(3-thienyl)-4H-indeno[1,2-c]pyrazol-3-yl]-(3,3-dimethylmorpholin-4-yl)methanone (300 mg, 614.26 μmol, 1 eq) and Pd(dppf)Cl2 (89.89 mg, 122.85 μmol, 0.2 eq). The mixture was stirred at 80° C. for 16 h under N₂ atmosphere. The reaction mixture was poured into H₂O (5 mL), and extracted with ethyl acetate (5 mL*3). The combined organic phases were dried over anhydrous Na₂SO₄ and filtered. The filtrate was concentrated in vacuo to give a residue that was purified by flash silica gel chromatography (ISCO®; 4 g SepaFlash® Silica Flash Column, Eluent of 0˜70% Petroleum ether gradient/Ethyl acetate @30 mL/min) to afford (3,3-dimethylmorpholin-4-yl)-[7-(5-cyano-3-pyridyl)-6-methoxy-1-(3-thienyl)-4H-indeno[1,2-c]pyrazol-3-yl]methanone (150 mg, 293.20 μmol, 48% yield) as a brown solid.

### Synthesis of 5-[3-(3,3-dimethylmorpholine-4-carbonyl)-6-methoxy-1-(3-thienyl)-4H-indeno[1,2-c]pyrazol-7-yl]pyridine-3-carboxamide

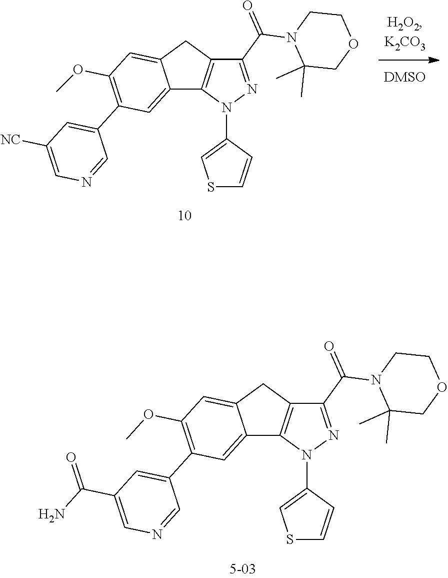 [SMILES: COc1cc2c(cc1-c1cncc(C#N)c1)-c1c(c(C(=O)N3CCOCC3(C)C)nn1-c1ccsc1)C2 ; COc1cc2c(cc1-c1cncc(C(N)=O)c1)-c1c(c(C(=O)N3CCOCC3(C)C)nn1-c1ccsc1)C2]

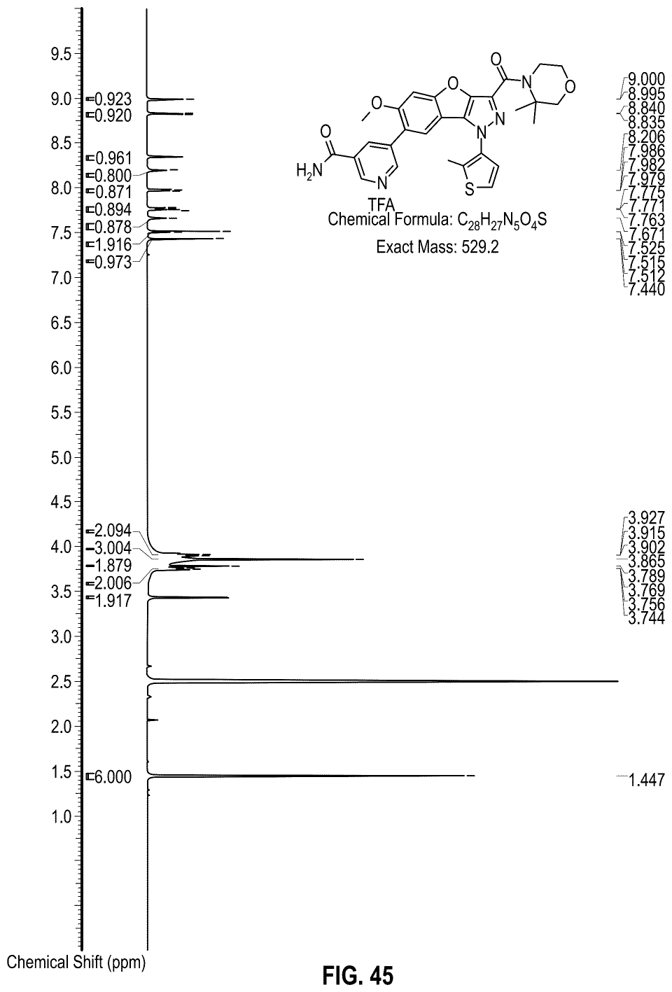

To a mixture of (3,3-dimethylmorpholin-4-yl)-[7-(5-cyano-3-pyridyl)-6-methoxy-1-(3-thienyl)-4H-indeno[1,2-c]pyrazol-3-yl]methanone (100 mg, 195.47 μmol, 1 eq) in DMSO (2 mL) was added K₂CO₃ (54.03 mg, 390.94 μmol, 2 eq) and H₂O₂ (410 mg, 3.62 mmol, 347.46 μL, 30% purity, 18.50 eq). The mixture was stirred at 15° C. for 15 min. The mixture was poured into saturated aqueous Na₂S₂O₃ (10 mL), and then stirred at 15° C. for another 1 hour, then extracted with ethyl acetate (10 mL*3). The combined organic phases were dried over anhydrous Na₂SO₄. It was filtered and the filtrate was concentrated in vacuo to give a residue that was purified by prep-HPLC (column: Phenomenex Luna C18 150*25 mm*10 um; mobile phase: [water (TFA)-ACN]; gradient: 28%-58% B over 9 min) followed by lyophilization to afford 5-[3-(3,3-dimethylmorpholine-4-carbonyl)-6-methoxy-1-(3-thienyl)-4H-indeno[1,2-c]pyrazol-7-yl]pyridine-3-carboxamide (99.06 mg, 153.91 μmol, 79% yield, 100% purity, TFA) as a yellow solid. LCMS (ESI): m/z [M+H] calcd for C₃₀H₂₈N₅O₆SF₃: 530.18; found: 530.2. ¹H NMR (400 MHz, DMSO-d₆) δ=9.00 (d, J=1.96 Hz, 1H), 8.84 (d, J=2.08 Hz, 1H), 8.37-8.32 (m, 1H), 8.21 (s, 1H), 7.98 (dd, J=3.00, 1.28 Hz, 1H), 7.77 (dd, J=5.01, 3.18 Hz, 1H), 7.67 (s, 1H), 7.55-7.50 (m, 2H), 7.44 (s, 1H), 3.92 (br s, 2H), 3.86 (s, 3H), 3.79 (s, 2H), 3.74-3.77 (m, 2H), 3.45-3.42 (m, 2H), 1.45 (s, 6H). FIG. 45 shows the nuclear magnetic resonance of Compound 5-03.

### Synthesis of 7-bromo-N-tert-butyl-6-methoxy-N-methyl-1-(3-thienyl)-4H-indeno[1,2-c]pyrazole-3-carboxamide

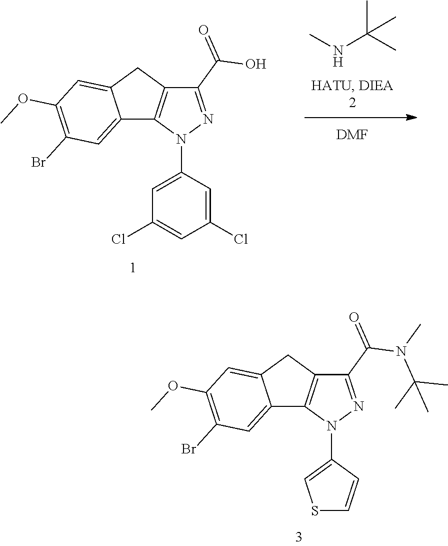

To a mixture of 7-bromo-6-methoxy-1-(3-thienyl)-4H-indeno[1,2-c]pyrazole-3-carboxylic acid (500 mg, 1.28 mmol, 1 eq) in DMF (5 mL) was added HATU (583.12 mg, 1.53 mmol, 1.2 eq) and DIEA (495.51 mg, 3.83 mmol, 667.81 μL, 3 eq). The mixture was stirred at 15° C. for 15 min, then N-2-dimethylpropan-2-amine (167.09 mg, 1.92 mmol, 229.84 μL, 1.5 eq) was added and the mixture stirred at 15° C. for a further16 h. The mixture was then poured into H₂O (20 mL), there were precipitates formed in the reaction mixture, the mixture was filtered and the filter cake was collected and dried in vacuo to afford 7-bromo-N-tert-butyl-6-methoxy-N-methyl-1-(3-thienyl)-4H-indeno[1,2-c]pyrazole-3-carboxamide (500 mg, 1.09 mmol, 84.98% yield) as a black solid.

### Synthesis of N-tert-butyl-6-methoxy-N-methyl-7-(1-methylpyrazol-3-yl)-1-(3-thienyl)-4H-indeno[1,2-c]pyrazole-3-carboxamide

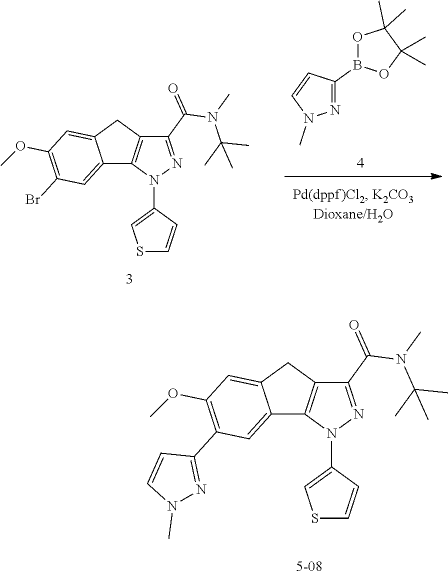 [SMILES: COc1cc2c(cc1Br)-c1c(c(C(=O)N(C)C(C)(C)C)nn1-c1ccsc1)C2 ; Cn1ccc(B2OC(C)(C)C(C)(C)O2)n1 ; COc1cc2c(cc1-c1ccn(C)n1)-c1c(c(C(=O)N(C)C(C)(C)C)nn1-c1ccsc1)C2]

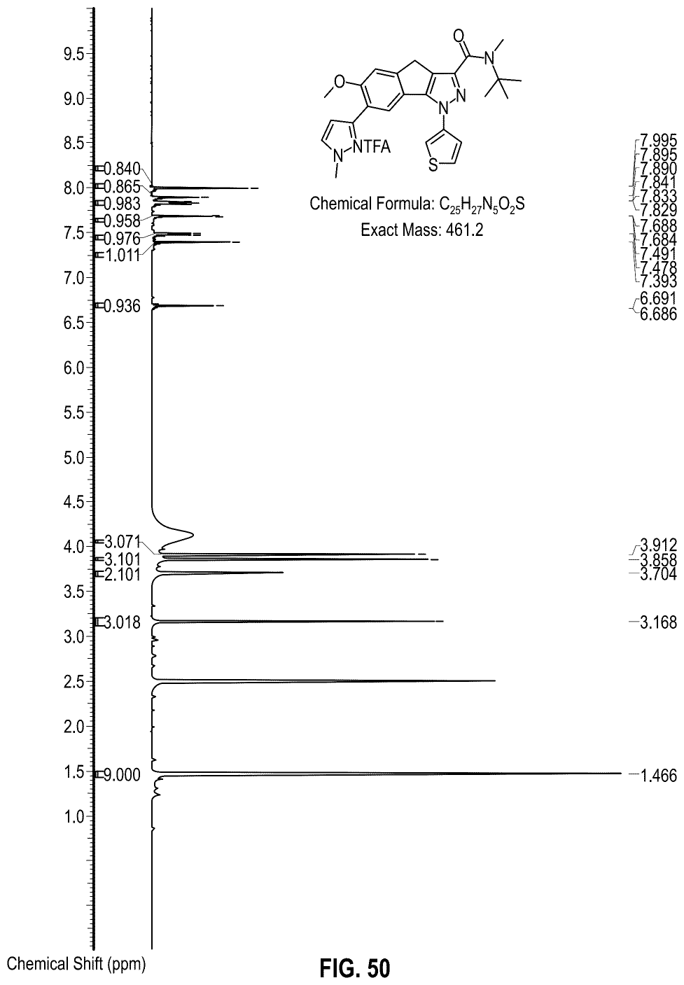

To a mixture of 1-methyl-3-(4,4,5,5-tetramethyl-1,3,2-dioxaborolan-2-yl)pyrazole (108.46 mg, 521.30 μmol, 1.2 eq) in dioxane (2.5 mL) and H₂O (0.5 mL) was added K₂CO₃ (180.12 mg, 1.30 mmol, 3 eq), 7-bromo-N-tert-butyl-6-methoxy-N-methyl-1-(3-thienyl)-4H-indeno [1, 2-c]pyrazole-3-carboxamide (200 mg, 434.42 μmol, 1 eq) and Pd(dppf)Cl₂ (63.57 mg, 86.88 μmol, 0.2 eq). The mixture was stirred at 80° C. for 16 h under N₂ atmosphere. The mixture was filtered and the filtrate was concentrated under vacuum to give a residue that was purified by prep-HPLC (column: Phenomenex Luna C18 150*25 mm*10 um; mobile phase: [water (TFA)-ACN]; gradient: 52%-82% B) followed by lyophilization to afford N-tert-butyl-6-methoxy-N-methyl-7-(1-methylpyrazol-3-yl)-1-(3-thienyl)-4H-indeno[1,2-c]pyrazole-3-carboxamide (56.24 mg, 93.80 μmol, 22% yield, 96% purity, TFA) as an off-white solid. LCMS (ESI): m/z [M+H] calcd for C₂₇H₂₈N₅O₄SF₃: 462.19; found: 462.2. ¹H NMR (400 MHz, DMSO-d₆) δ=8.17 (s, 1H), 7.87 (s, 2H), 7.80 (s, 1H), 7.70 (d, J=1.47 Hz, 1H), 7.41 (s, 1H), 6.72 (d, J=1.83 Hz, 1H), 3.94 (s, 3H), 3.86 (s, 3H), 3.73 (s, 2H), 3.17 (s, 3H), 1.48 (s, 9H). FIG. 50 shows the nuclear magnetic resonance of Compound 5-08.

### Synthesis of N-tert-butyl-7-(5-cyano-3-pyridyl)-6-methoxy-N-methyl-1-(3-thienyl)-4H-indeno[1,2-c]pyrazole-3-carboxamide

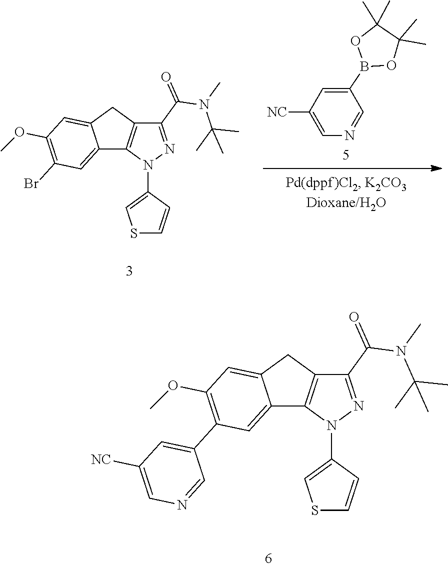 [SMILES: COc1cc2c(cc1Br)-c1c(c(C(=O)N(C)C(C)(C)C)nn1-c1ccsc1)C2 ; CC1(C)OB(c2cncc(C#N)c2)OC1(C)C ; COc1cc2c(cc1-c1cncc(C#N)c1)-c1c(c(C(=O)N(C)C(C)(C)C)nn1-c1ccsc1)C2]

To a mixture of 5-(4,4,5,5-tetramethyl-1,3,2-dioxaborolan-2-yl) pyridine-3-carbonitrile (179.90 mg, 781.95 μmol, 1.2 eq) in dioxane (2.5 mL) and H₂O (0.5 mL) was added K₂CO₃ (270.18 mg, 1.95 mmol, 3 eq), 7-bromo-N-tert-butyl-6-methoxy-N-methyl-1-(3-thienyl)-4H-indeno [1,2-c]pyrazole-3-carboxamide (300 mg, 651.63 μmol, 1 eq) and Pd(dppf)Cl₂ (95.36 mg, 130.33 μmol, 0.2 eq). The mixture was stirred at 80° C. for 16 h under N₂ atmosphere. The reaction mixture was poured into H₂O (5 mL) and ethyl acetate (5 mL), then the mixture was separated. The aqueous phase was extracted with ethyl acetate (5 mL*3). The combined organic phases were dried over anhydrous Na2SO4. It was filtered and the filtrate was concentrated in vacuo to give a residue that was purified by flash silica gel chromatography (ISCO®; 4 g SepaFlash® Silica Flash Column, Eluent of 0˜70% Petroleum ether gradient/Ethylacetate@30 mL/min) to afford N-tert-butyl-7-(5-cyano-3-pyridyl)-6-methoxy-N-methyl-1-(3-thienyl)-4H-indeno[1,2-c]pyrazole-3-carboxamide (200 mg, 413.58 μmol, 63% yield) as a brown solid.

### Synthesis of N-tert-butyl-7-(5-carbamoyl-3-pyridyl)-6-methoxy-N-methyl-1-(3-thienyl)-4H-indeno[1,2-c]pyrazole-3-carboxamide

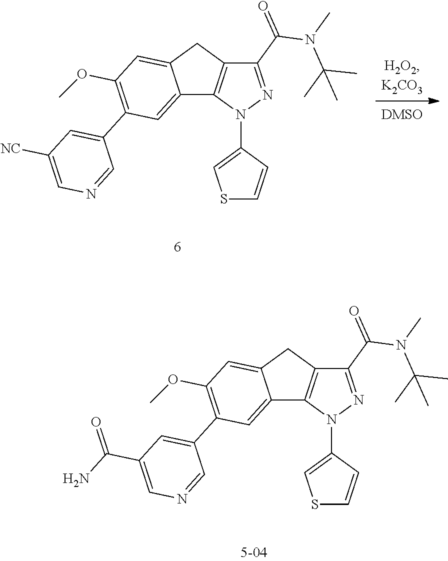 [SMILES: COc1cc2c(cc1-c1cncc(C#N)c1)-c1c(c(C(=O)N(C)C(C)(C)C)nn1-c1ccsc1)C2 ; COc1cc2c(cc1-c1cncc(C(N)=O)c1)-c1c(c(C(=O)N(C)C(C)(C)C)nn1-c1ccsc1)C2]

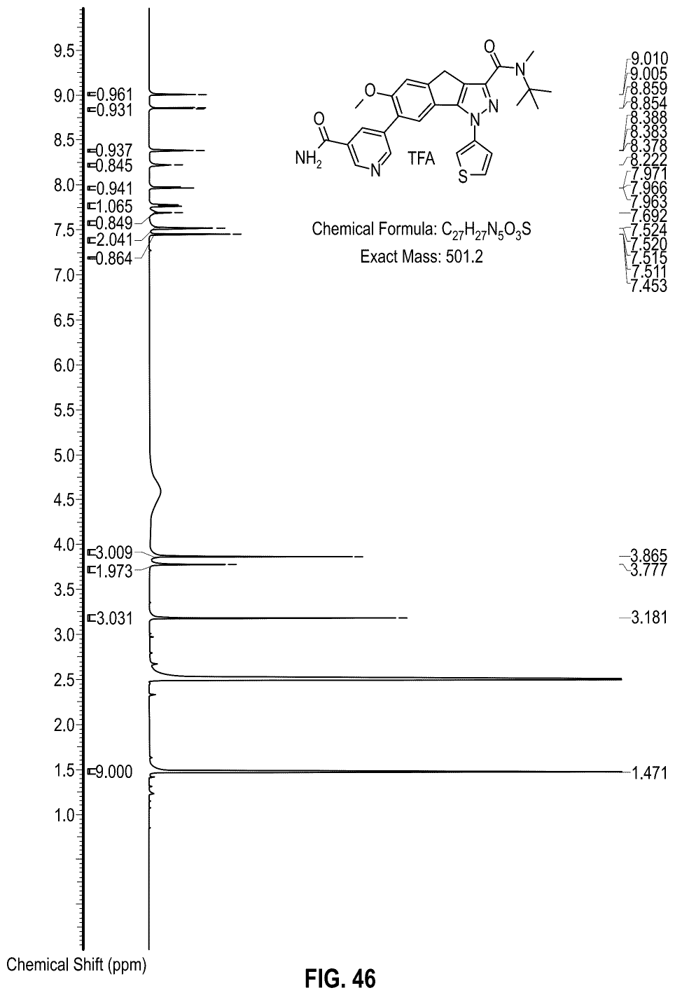

To a mixture of N-tert-butyl-7-(5-cyano-3-pyridyl)-6-methoxy-N-methyl-1-(3-thienyl)-4H-indeno[1,2-c]pyrazole-3-carboxamide (150 mg, 310.18 μmol, 1 eq) in DMSO (2 mL) was added H₂O₂ (140 mg, 1.23 mmol, 118.64 μL, 30% purity, 3.98 eq) and K₂CO₃ (85.74 mg, 620.37 μmol, 2 eq). The mixture was stirred at 15° C. for 15 min. The mixture was poured into saturated aqueous Na₂S₂O₃ (10 mL), and the mixture was stirred at 15° C. for another 1 hour. The resulting mixture was extracted with ethyl acetate (10 mL*3), the combined organic phases were concentrated under vacuum to give a residue that was purified by prep-HPLC (column: Phenomenex Luna C18 150*25 mm*10 um; mobile phase: [water (TFA)-ACN]; gradient: 35%-65% B over 9 min) followed by lyophilization to afford N-tert-butyl-7-(5-carbamoyl-3-pyridyl)-6-methoxy-N-methyl-1-(3-thienyl)-4H-indeno[1,2-c]pyrazole-3-carboxamide (94.35 mg, 147.13 μmol, 47% yield, 96% purity, TFA) as a yellow solid. LCMS (ESI): m z (M+H) calcd for C₂₉H₂₈N₅O₅SF₃: 502.18; found: 502.2. ¹H NMR (400 MHz, DMSO-d₆) δ=9.01 (d, J=2.00 Hz, 1H), 8.86 (d, J=2.00 Hz, 1H), 8.38 (t, J=2.06 Hz, 1H), 8.22 (s, 1H), 7.97 (dd, J=3.19, 1.44 Hz, 1H), 7.80-7.74 (m, 1H), 7.69 (s, 1H), 7.52 (dt, J=3.38, 1.56 Hz, 2H), 7.45 (s, 1H), 3.86 (s, 3H), 3.78 (s, 2H) 3.18 (s, 3H), 1.47 (s, 9H). FIG. 46 shows the nuclear magnetic resonance of Compound 5-04.

### Example 7

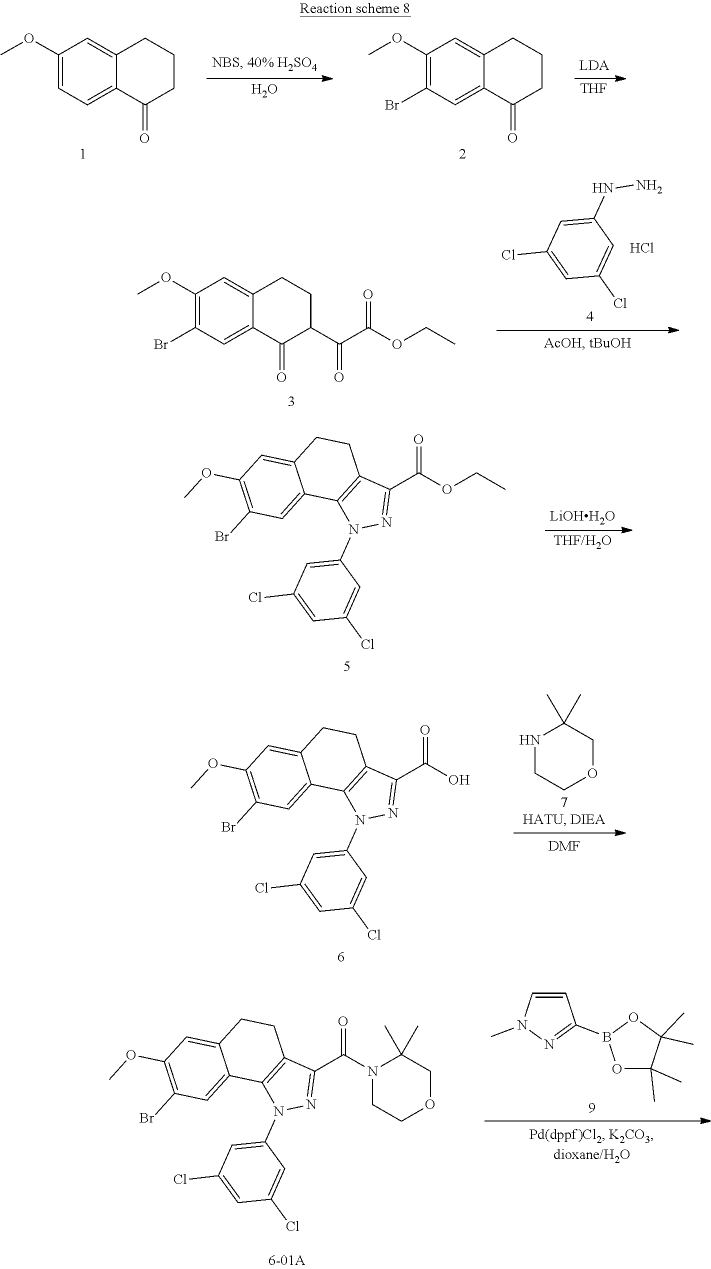

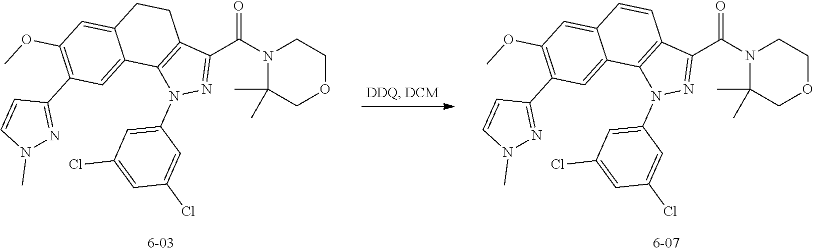 [SMILES: COc1cc2c(cc1-c1ccn(C)n1)-c1c(c(C(=O)N3CCOCC3(C)C)nn1-c1cc(Cl)cc(Cl)c1)CC2 ; COc1cc2ccc3c(C(=O)N4CCOCC4(C)C)nn(-c4cc(Cl)cc(Cl)c4)c3c2cc1-c1ccn(C)n1]

### Synthesis of 7-bromo-6-methoxy-tetralin-1-one

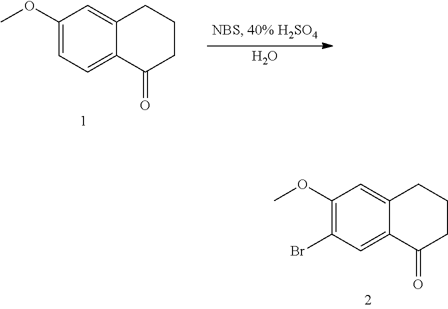

To a mixture of 6-methoxytetralin-1-one (20 g, 113.50 mmol, 1 eq) and NBS (20.20 g, 113.50 mmol, 1 eq) in H₂O (200 mL) was added H₂SO₄ (55.66 g, 227.00 mmol, 30.25 mL, 40% purity, 2 eq) at 0° C. The mixture was then stirred at 60° C. for 5 h. The mixture was cooled to r.t. and filtered and the filter cake was collected to give the crude product. The crude product was purified by flash silica gel chromatography (ISCO®; 220 g SepaFlash® Silica Flash Column, Eluent of 0˜18% Ethyl acetate/Petroleum ether gradient @60 mL/min). Compound 7-bromo-6-methoxy-tetralin-1-one (4.2 g, 16.46 mmol, 15% yield) was obtained as a white solid. ¹H NMR (400 MHz, CHLOROFORM-d) δ=8.27-8.16 (m, 1H), 6.70 (br s, 1H), 3.99-3.91 (m, 3H), 2.96-2.86 (m, 2H), 2.66-2.55 (m, 2H), 2.19-2.07 (m, 2H).

### Synthesis of ethyl 2-(7-bromo-6-methoxy-1-oxo-tetralin-2-yl)-2-oxo-acetate

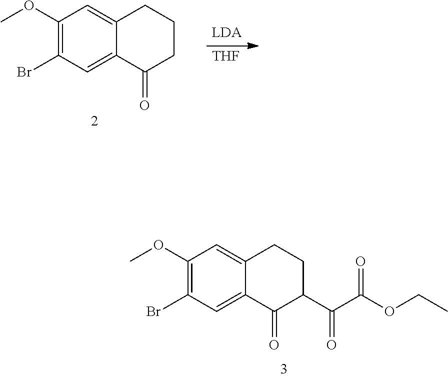 [SMILES: COc1cc2c(cc1Br)C(=O)CCC2 ; CCOC(=O)C(=O)C1CCc2cc(OC)c(Br)cc2C1=O ; "THF"]

To a solution of 7-bromo-6-methoxy-tetralin-1-one (4 g, 15.68 mmol, 1 eq) in THF (60 mL) was added dropwise LDA (2 M, 10.19 mL, 1.3 eq) at −70° C. over 10 min. After addition, the mixture was stirred at −70° C. for 0.5 h, and then diethyl oxalate (3.44 g, 23.52 mmol, 3.21 mL, 1.5 eq) in THF (10 mL) added dropwise at −70° C. over 10 min. The resulting mixture was stirred at 0° C. for 16 h. The mixture was poured into ice-water (200 mL) and the reaction mixture was acidified to pH=6 with 1 M HCl aqueous solution. The aqueous phase was extracted with ethyl acetate (200 mL*3) and the combined organic phases washed with brine (500 mL) and dried over anhydrous Na₂SO₄. After filtration, the filtrate was concentrated in vacuo to provide a residue that was purified by prep-HPLC (column: Phenomenex Luna C18 (250*70 mm, 10 um); mobile phase: [water (FA)-ACN]; gradient: 55%-85% B over 20 min). Compound ethyl 2-(7-bromo-6-methoxy-1-oxo-tetralin-2-yl)-2-oxo-acetate (3.6 g, 10.14 mmol, 65% yield) was obtained as a yellow solid. ¹H NMR (400 MHz, CHLOROFORM-d) δ=8.19 (s, 1H), 6.72 (s, 1H), 4.38 (q, J=7.1 Hz, 2H), 3.97 (s, 3H), 3.00-2.93 (m, 2H), 2.89-2.82 (m, 2H), 1.41 (t, J=7.1 Hz, 3H).

### Synthesis of ethyl 8-bromo-1-(3,5-dichlorophenyl)-7-methoxy-4,5-dihydrobenzo[g]indazole-3-carboxylate

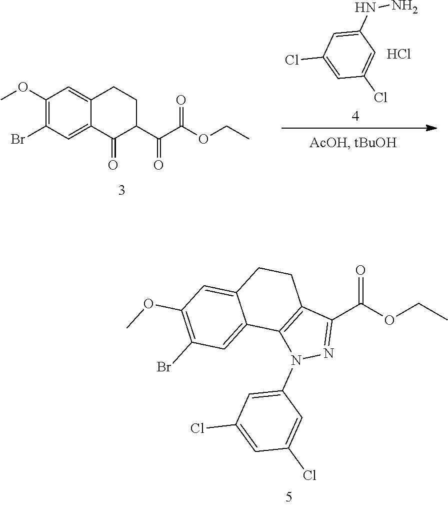 [SMILES: CCOC(=O)C(=O)C1CCc2cc(OC)c(Br)cc2C1=O ; NNc1cc(Cl)cc(Cl)c1 ; Cl ; CCOC(=O)c1nn(-c2cc(Cl)cc(Cl)c2)c2c1CCc1cc(OC)c(Br)cc1-2]

A mixture of ethyl 2-(7-bromo-6-methoxy-1-oxo-tetralin-2-yl)-2-oxo-acetate (3.5 g, 9.85 mmol, 1 eq), (3,5-dichlorophenyl) hydrazine; hydrochloride (2.31 g, 10.84 mmol, 1.1 eq) in EtOH (40 mL) and AcOH (5.92 g, 98.54 mmol, 5.64 mL, 10 eq) was stirred at 80° C. for 5 h under N₂ atmosphere. The mixture was cooled to r.t. and filtered and the filter cake was collected and dried in vacuo to afford ethyl 8-bromo-1-(3,5-dichlorophenyl)-7-methoxy-4,5-dihydrobenzo[g]indazole-3-carboxylate (4.5 g, crude) as a white solid. ¹H NMR (400 MHz, DMSO-d₆) δ=7.94-7.88 (m, 1H), 7.74 (d, J=1.8 Hz, 2H), 7.23 (s, 1H), 6.84 (s, 1H), 4.32 (q, J=7.0 Hz, 2H), 3.88 (s, 3H), 2.96 (br s, 4H), 1.32 (t, J=7.1 Hz, 3H).

### Synthesis of 8-bromo-1-(3,5-dichlorophenyl)-7-methoxy-4,5-dihydrobenzo[g]indazole-3-carboxylic acid

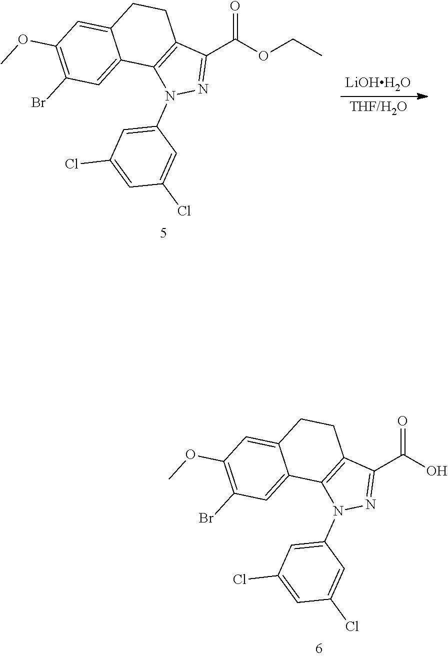

A mixture of ethyl 8-bromo-1-(3,5-dichlorophenyl)-7-methoxy-4,5-dihydrobenzo[g]indazole-3-carboxylate (4.5 g, 9.07 mmol, 1 eq) and LiOH. H₂O (1.52 g, 36.28 mmol, 10 mL, 4 eq) in EtOH (40 mL) was stirred at 25° C. for 16 h under N₂ atmosphere. The reaction mixture was acidified to pH=6 with 1 M HCl aqueous solution. There were precipitates formed in the reaction mixture and the mixture was filtered and the filter cake was collected and dried in vacuo to afford 8-bromo-1-(3, 5-dichlorophenyl)-7-methoxy-4, 5-dihydrobenzo[g]indazole-3-carboxylic acid (4 g, crude) as a white solid. LCMS (ESI): m/z [M+H] calcd for C₁₉H₁₄BrCl₂N₂O₃: 466.95; found: 467.0/468.9.

### Synthesis of [8-bromo-1-(3,5-dichlorophenyl)-7-methoxy-4,5-dihydrobenzo[g]indazol-3-yl]-(3,3-dimethylmorpholin-4-yl)methanone

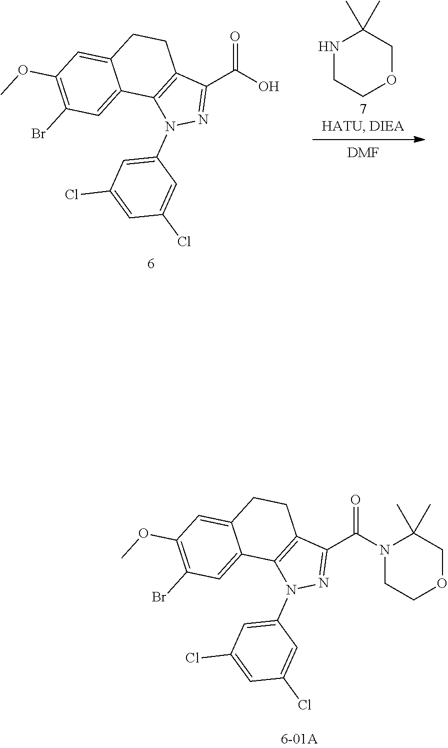

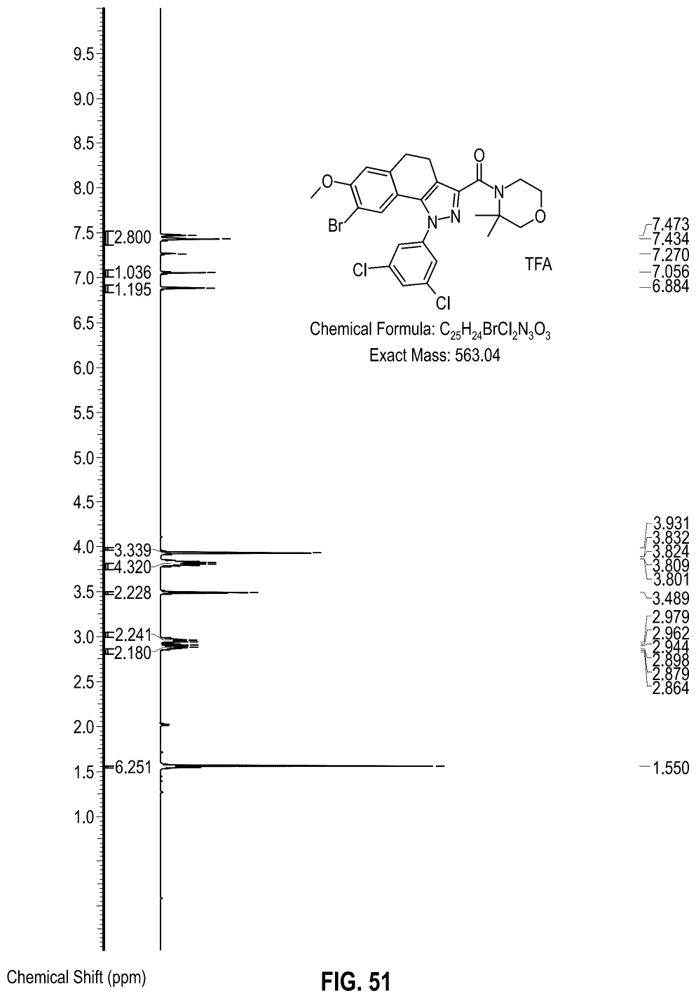

To a solution of 8-bromo-1-(3,5-dichlorophenyl)-7-methoxy-4,5-dihydrobenzo[g]indazole-3-carboxylic acid (1 g, 2.14 mmol, 1 eq) in DMF (10 mL) was added HATU (1.22 g, 3.20 mmol, 1.5 eq) and DIEA (828.25 mg, 6.41 mmol, 1.12 mL, 3 eq) at 25° C. After addition, the mixture was stirred at this temperature for 0.5 h, and then 3,3-dimethylmorpholine (246.03 mg, 2.14 mmol, 1 eq) was added and the resulting mixture was stirred at 25° C. for 2 h. The mixture was then poured into ice-water (30 mL) and stirred for 3 min. The aqueous phase was extracted with ethyl acetate (20 mL*3). The combined organic phase was washed with brine (50 mL), and dried over anhydrous Na2SO4. The mixture was filtered and the filtrate was concentrated in vacuo to provide a residue that was purified by flash silica gel chromatography (ISCO®; 20 g SepaFlash® Silica Flash Column, Eluent of 0˜30% Ethyl acetate/Petroleum ether gradient @45 mL/min) to get the crude product. The crude product was further purified by prep-HPLC (column: Phenomenex Luna C18 150*25 mm*10 um; mobile phase: [water (TFA) -ACN]; gradient: 70%-100% B over 9 min). Compound [8-bromo-1-(3,5-dichlorophenyl)-7-methoxy-4,5-dihydrobenzo[g]indazol-3-yl]-(3,3-dimethylmorpholin-4-yl)methanone (1.05 g, 1.55 mmol, 72% yield, TFA salt) was obtained as a white solid. LCMS (ESI): m/z [M+H] calcd for C₂₅H₂₅BrCl₂N₃O₃: 564.04; found: 564.2. ¹H NMR (400 MHz, CHLOROFORM-d) δ=7.52-7.37 (m, 3H), 7.06 (s, 1H), 6.88 (s, 1H), 3.93 (s, 3H), 3.87-3.77 (m, 4H), 3.49 (s, 2H), 3.00-2.93 (m, 2H), 2.91-2.84 (m, 2H), 1.55 (s, 6H). FIG. 51 shows the nuclear magnetic resonance of Compound 6-01A.

### Synthesis of [1-(3,5-dichlorophenyl)-7-methoxy-8-(1-methylpyrazol-3-yl)-4,5-dihydrobenzo [g]indazol-3-yl]-(3,3-dimethylmorpholin-4-yl)methanone

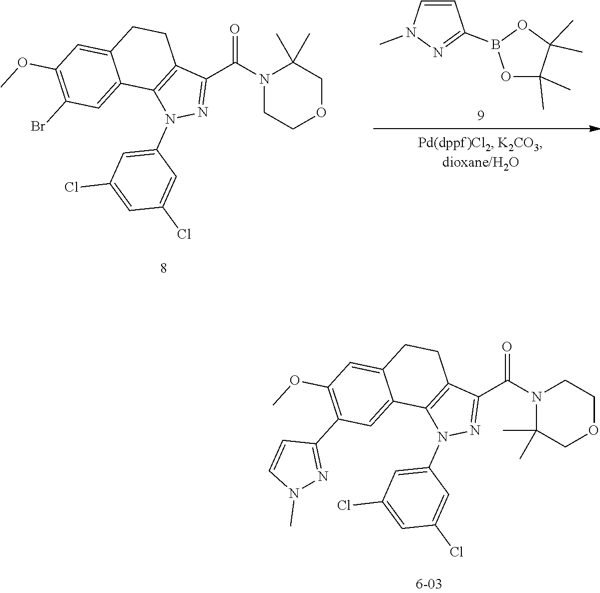

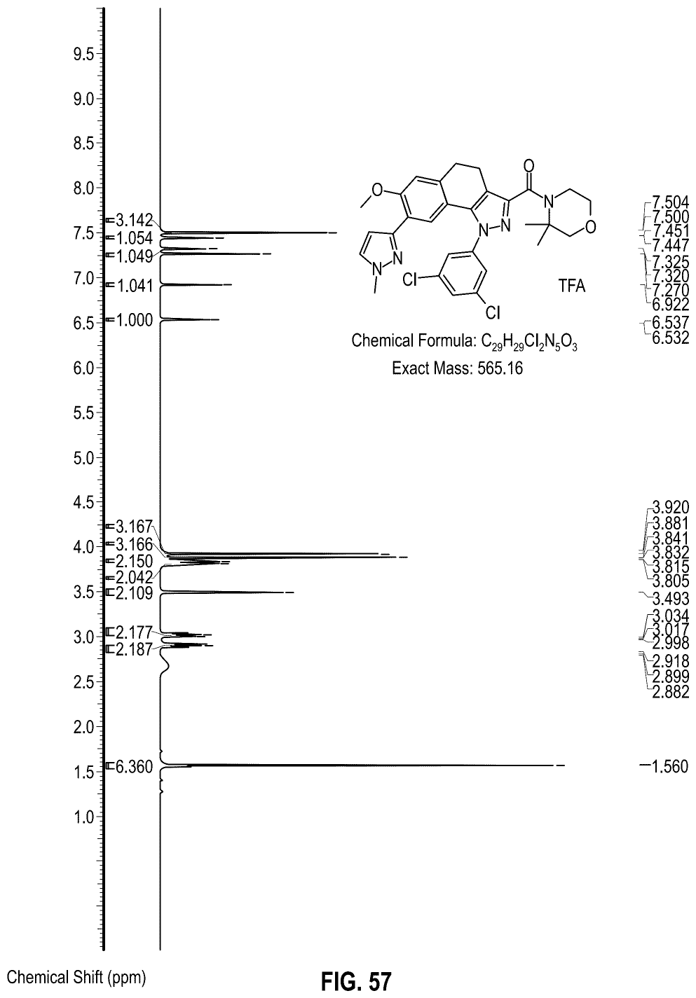

A mixture of 1-methyl-3-(4,4,5,5-tetramethyl-1,3,2-dioxaborolan-2-yl)pyrazole (40.49 mg, 194.59 μmol, 1.1 eq), [8-bromo-1-(3,5-dichlorophenyl)-7-methoxy-4,5-dihydrobenzo[g]indazol-3-yl]-(3,3-dimethylmorpholin-4-yl)methanone (0.1 g, 176.90 μmol, 1 eq), K₂CO₃ (48.90 mg, 353.80 μmol, 2 eq) and Pd(dppf)Cl₂ (12.94 mg, 17.69 μmol, 0.1 eq) in dioxane (2 mL) and H₂O (0.2 mL) was degassed and purged with N₂ 3 times, then heated at 80° C. for 16 h under N₂. The residue was diluted with EtOAc (10 mL) and filtered through a celite pad. The filtrate was concentrated to provide a residue that was purified by flash silica gel chromatography (ISCO®; 10 g SepaFlash® Silica Flash Column, Eluent of 0˜35% Ethyl acetate/Petroleum ether gradient @45 mL/min) to get the crude product. The crude product was further purified by prep-HPLC (column: Phenomenex Luna C18 150*25 mm*10 um; mobile phase: [water (TFA) -ACN]; gradient: 62%-92% B over 9 min). Compound [1-(3,5-dichlorophenyl)-7-methoxy-8-(1-methylpyrazol-3-yl)-4,5-dihydrobenzo[g]indazol-3-yl]-(3,3-dimethylmorpholin-4-yl)methanone (30 mg, 44.09 μmol, 25% yield, TFA) was obtained as a white solid. LCMS (ESI): m/z [M+H] calcd for C₂₉H₃₀Cl₂N₅O₃: 566.16; found: 566.3. ¹H NMR (400 MHz, CHLOROFORM-d) δ=7.50 (d, J=1.4 Hz, 3H), 7.47-7.43 (m, 1H), 7.32 (d, J=1.9 Hz, 1H), 6.92 (s, 1H), 6.53 (d, J=2.1 Hz, 1H), 3.92 (s, 3H), 3.88 (s, 3H), 3.84 (br d, J=3.6 Hz, 2H), 3.82-3.78 (m, 2H), 3.49 (s, 2H), 3.06-2.97 (m, 2H), 2.94-2.86 (m, 2H), 1.56 (s, 6H). FIG. 57 shows the nuclear magnetic resonance of Compound 6-03.

### Synthesis of [1-(3,5-dichlorophenyl)-7-methoxy-8-(1-methylpyrazol-3-yl)benzo[g]indazol-3-yl]-(3,3-dimethylmorpholin-4-yl)methanone

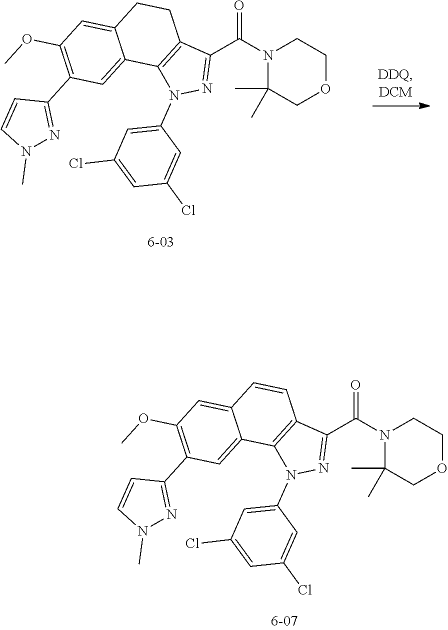 [SMILES: COc1cc2c(cc1-c1ccn(C)n1)-c1c(c(C(=O)N3CCOCC3(C)C)nn1-c1cc(Cl)cc(Cl)c1)CC2 ; COc1cc2ccc3c(C(=O)N4CCOCC4(C)C)nn(-c4cc(Cl)cc(Cl)c4)c3c2cc1-c1ccn(C)n1]

A mixture of [1-(3, 5-dichlorophenyl)-7-methoxy-8-(1-methylpyrazol-3-yl)-4,5-dihydrobenzo[g]indazol-3-yl]-(3, 3-dimethylmorpholin-4-yl)methanone (90 mg, 158.88 μmol, 1 eq) and DDQ (144.26 mg, 635.51 μmol, 4 eq) in dioxane (2 mL) was stirred at 80° C. for 16 h under N₂ atmosphere. The product was poured into ice-water (20 mL) and quenched with sat. aq. Na₂SO₃ (10 mL). The aqueous phase was extracted with ethyl acetate (10 mL*3) and the combined organic phases washed with brine (20 mL) and dried with anhydrous Na₂SO₄. The mixture was filtered and the filtrate was concentrated in vacuo to provide a residue that was purified by prep-HPLC (column: Phenomenex Luna C18 150*25 mm*10 um; mobile phase: [water (TFA) -ACN]; gradient: 66%-96% B over 9 min). Compound [1-(3,5-dichlorophenyl)-7-methoxy-8-(1-methylpyrazol-3-yl)benzo[g]indazol-3-yl]-(3,3-dimethylmorpholin-4-yl)methanone (50 mg, 73.69 μmol, 46% yield, TFA) was obtained as a white solid. LCMS (ESI): m/z [M+H] calcd for C₂₉H₂₈Cl₂N₅O₃: 564.15; found: 564.1. ¹H NMR (400 MHz, CHLOROFORM-d) δ=8.32 (s, 1H), 7.85 (d, J=8.9 Hz, 1H), 7.55 (d, J=1.8 Hz, 2H), 7.53-7.49 (m, 2H), 7.30 (s, 2H), 6.64 (d, J=2.1 Hz, 1H), 3.96 (s, 3H), 3.87 (s, 3H), 3.80 (s, 4H), 3.47 (s, 2H), 1.55 (s, 6H). FIG. 57 shows the nuclear magnetic resonance of Compound 6-07.

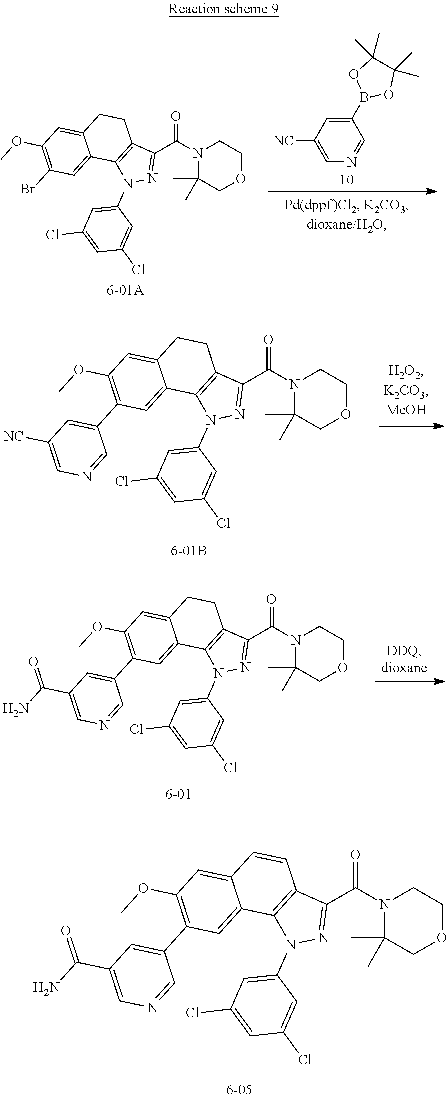 [SMILES: COc1cc2c(cc1Br)-c1c(c(C(=O)N3CCOCC3(C)C)nn1-c1cc(Cl)cc(Cl)c1)CC2 ; CC1(C)OB(c2cncc(C#N)c2)OC1(C)C ; COc1cc2c(cc1-c1cncc(C#N)c1)-c1c(c(C(=O)N3CCOCC3(C)C)nn1-c1cc(Cl)cc(Cl)c1)CC2 ; COc1cc2c(cc1-c1cncc(C(N)=O)c1)-c1c(c(C(=O)N3CCOCC3(C)C)nn1-c1cc(Cl)cc(Cl)c1)CC2 ; COc1cc2ccc3c(C(=O)N4CCOCC4(C)C)nn(-c4cc(Cl)cc(Cl)c4)c3c2cc1-c1cncc(C(N)=O)c1]

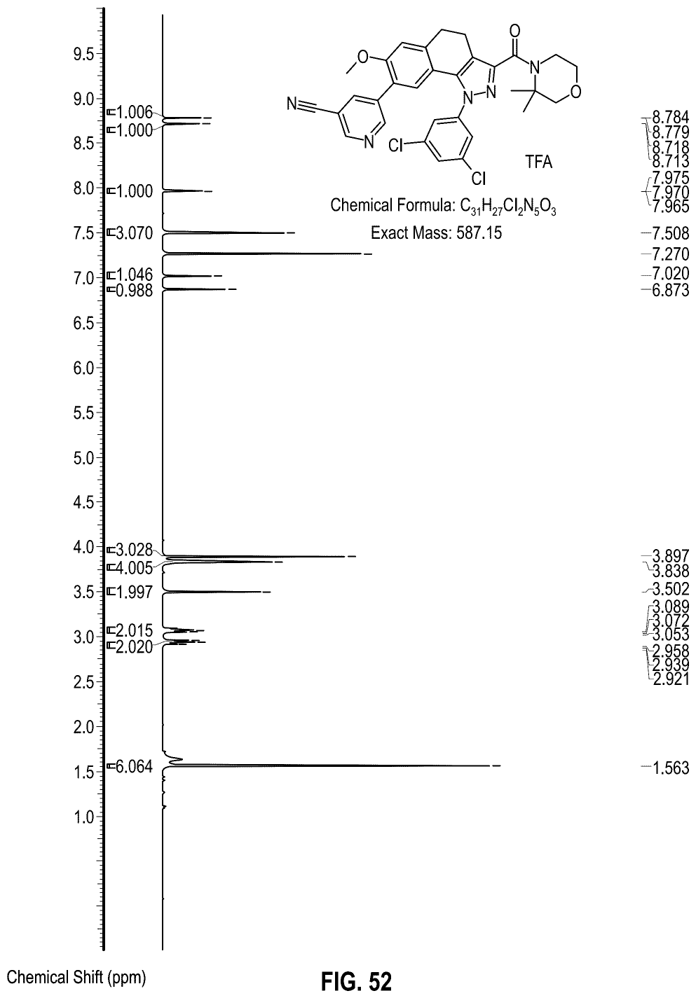

### Synthesis of 5-[1-(3,5-dichlorophenyl)-3-(3,3-dimethylmorpholine-4-carbonyl)-7-methoxy-4,5-dihydrobenzo[g]indazol-8-yl]pyridine-3-carbonitrile

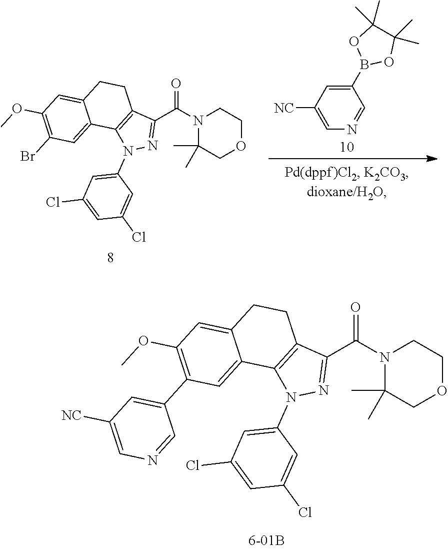 [SMILES: COc1cc2c(cc1Br)-c1c(c(C(=O)N3CCOCC3(C)C)nn1-c1cc(Cl)cc(Cl)c1)CC2 ; CC1(C)OB(c2cncc(C#N)c2)OC1(C)C ; COc1cc2c(cc1-c1cncc(C#N)c1)-c1c(c(C(=O)N3CCOCC3(C)C)nn1-c1cc(Cl)cc(Cl)c1)CC2]

[8-bromo-1-(3,5-dichlorophenyl)-7-methoxy-4,5-dihydrobenzo[g]indazol-3-yl]-(3,3-dimethylmorpholin-4-yl)methanone (0.5 g, 884.51 μmol, 1 eq), 5-(4,4,5,5-tetramethyl-1,3,2-dioxaborolan-2-yl)pyridine-3-carbonitrile (223.85 mg, 972.96 μmol, 1.1 eq), K₂CO₃ (244.49 mg, 1.77 mmol, 2 eq) and Pd(dppf)Cl₂ (129.44 mg, 176.90 μmol, 0.2 eq) in dioxane (5 mL) and H₂O (0.5 mL) was degassed and purged with N_{2 3} times, then heated at 80° C. for 16 h under N₂. The mixture was diluted with EtOAc (40 mL) and then filtered. The filtrate was concentrated to provide a residue that was purified by flash silica gel chromatography (ISCO®; 12 g SepaFlash® Silica Flash Column, Eluent of 0˜40% Ethyl acetate/Petroleum ether gradient @40 mL/min) to get the crude product. The crude product was further purified by prep-HPLC (column: Phenomenex Luna C18 150*25 mm*10 um; mobile phase: [water (TFA) -ACN]; gradient: 65%-95% B over 9 min). Compound 5-[1-(3,5-dichlorophenyl)-3-(3,3-dimethylmorpholine-4-carbonyl)-7-methoxy-4,5-dihydrobenzo[g]indazol-8-yl]pyridine-3-carbonitrile (340 mg, 483.98 μmol, 55% yield, TFA) was obtained as a white solid. LCMS (ESI): m/z [M+H] calcd for C31H28C12N5O3:588.15; found: 588.2. ¹H NMR (400 MHz, CHLOROFORM-d) δ=8.78 (d, J=1.9 Hz, 1H), 8.72 (d, J=2.1 Hz, 1H), 7.97 (t, J=2.0 Hz, 1H), 7.51 (s, 3H), 7.02 (s, 1H), 6.87 (s, 1H), 3.90 (s, 3H), 3.84 (s, 4H), 3.50 (s, 2H), 3.11-3.04 (m, 2H), 2.98-2.91 (m, 2H), 1.56 (s, 6H). FIG. 52 shows the nuclear magnetic resonance of Compound 6-01B.

### Synthesis of 5-[1-(3,5-dichlorophenyl)-3-(3,3-dimethylmorpholine-4-carbonyl)-7-methoxy-4,5-dihydrobenzo[g]indazol-8-yl]pyridine-3-carboxamide

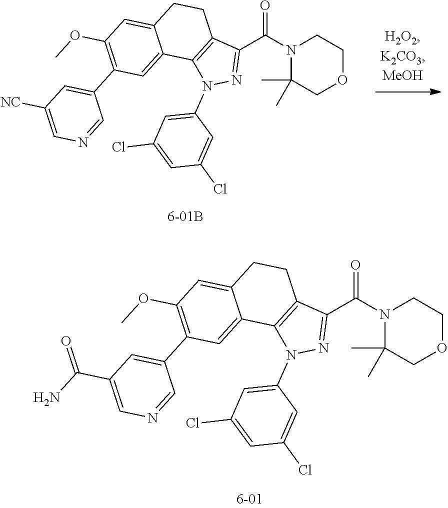 [SMILES: COc1cc2c(cc1-c1cncc(C#N)c1)-c1c(c(C(=O)N3CCOCC3(C)C)nn1-c1cc(Cl)cc(Cl)c1)CC2 ; COc1cc2c(cc1-c1cncc(C(N)=O)c1)-c1c(c(C(=O)N3CCOCC3(C)C)nn1-c1cc(Cl)cc(Cl)c1)CC2]

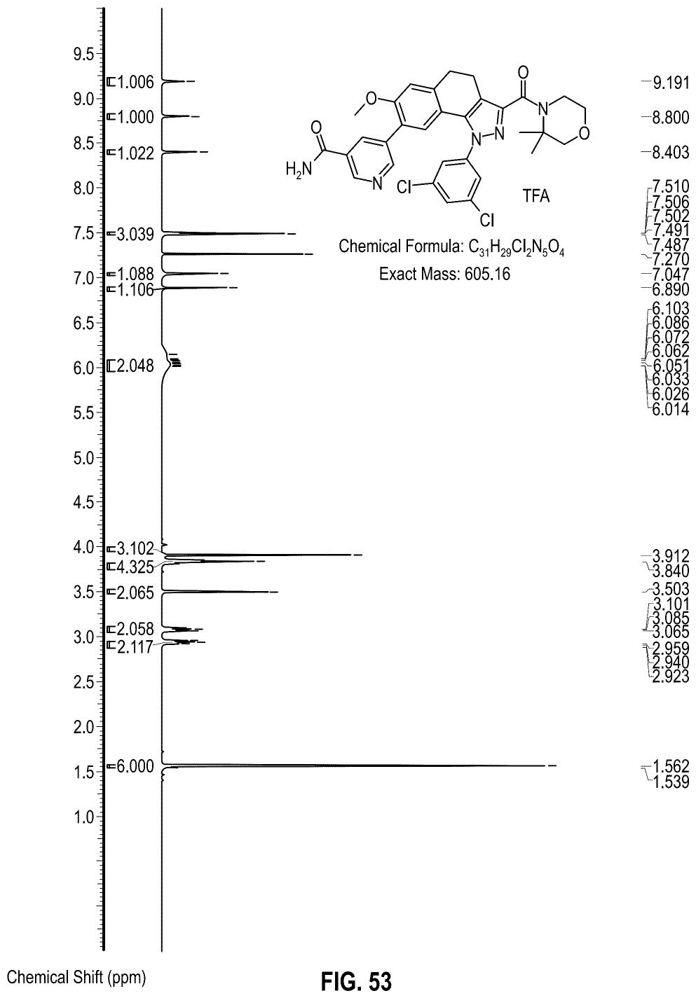

To a solution of 5-[1-(3,5-dichlorophenyl)-3-(3,3-dimethylmorpholine-4-carbonyl)-7-methoxy-4,5-dihydrobenzo[g]indazol-8-yl]pyridine-3-carbonitrile (0.3 g, 509.79 μmol, 1 eq) in MeOH (5 mL) was added K₂CO₃ (3 M, 339.86 μL, 2 eq) and H₂O₂ (0.39 g, 3.44 mmol, 330.51 μL, 30% purity, 6.75 eq). The mixture was stirred at 25° C. for 16 h. The reaction was quenched with sat. Na₂SO₃ (10 mL) and the mixture was extracted with EtOAc (10 mL*3), the combined organic phases were washed with brine (15 mL) and concentrated to provide a residue that was purified by prep-HPLC (column: Phenomenex Luna C18 150*40 mm*15 um; mobile phase: [water (TFA)-ACN]; gradient: 40%-70% B over 15 min). Compound 5-[1-(3,5-dichlorophenyl)-3-(3,3-dimethylmorpholine-4-carbonyl)-7-methoxy-4,5-dihydrobenzo[g]indazol-8-yl]pyridine-3-carboxamide (120 mg, 166.55 μmol, 33% yield, TFA) was obtained as a white solid. LCMS (ESI): m/z [M+H] calcd for C31H30Cl2N5O4: 606.16; found: 606.2. ¹H NMR (400 MHz, CHLOROFORM-d) δ=9.19 (s, 1H), 8.80 (s, 1H), 8.40 (s, 1H), 7.53-7.47 (m, 3H), 7.05 (s, 1H), 6.89 (s, 1H), 6.12-5.95 (m, 2H), 3.91 (s, 3H), 3.84 (s, 4H), 3.50 (s, 2H), 3.12-3.05 (m, 2H), 2.98-2.91 (m, 2H), 1.56 (s, 6H). FIG. 53 shows the nuclear magnetic resonance of Compound 6-01.

### Synthesis of 5-[1-(3,5-dichlorophenyl)-3-(3,3-dimethylmorpholine-4-carbonyl)-7-methoxy-benzo[g]indazol-8-yl]pyridine-3-carboxamide

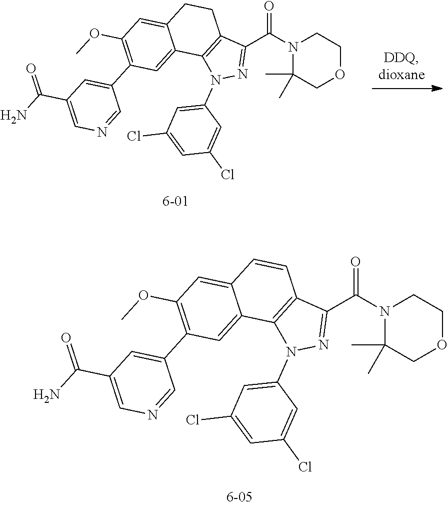 [SMILES: COc1cc2c(cc1-c1cncc(C(N)=O)c1)-c1c(c(C(=O)N3CCOCC3(C)C)nn1-c1cc(Cl)cc(Cl)c1)CC2 ; COc1cc2ccc3c(C(=O)N4CCOCC4(C)C)nn(-c4cc(Cl)cc(Cl)c4)c3c2cc1-c1cncc(C(N)=O)c1]

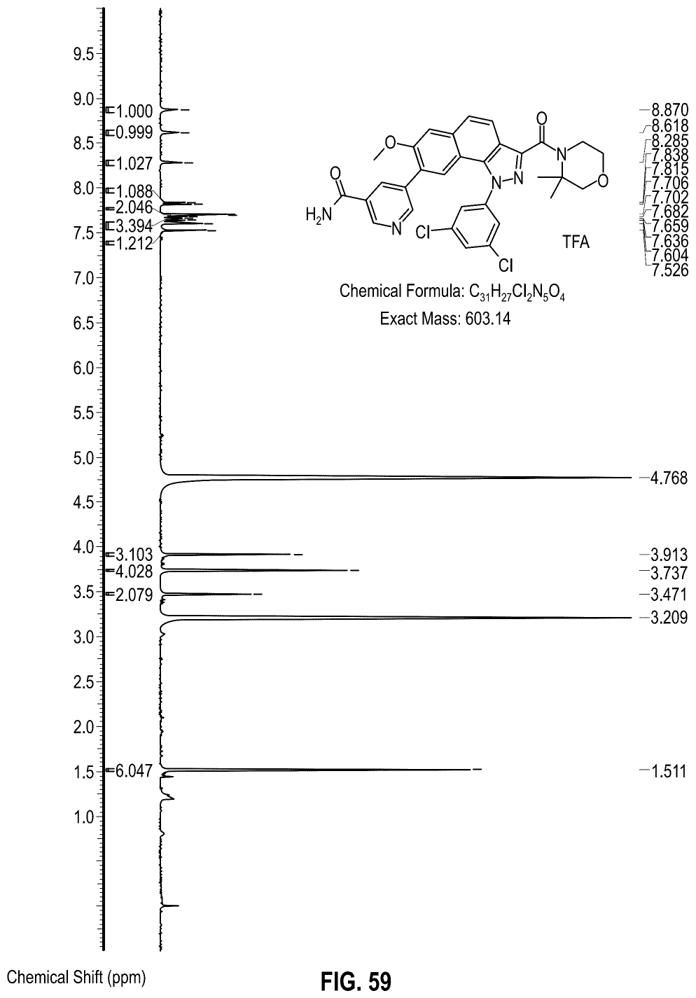

A mixture of 5-[1-(3,5-dichlorophenyl)-3-(3,3-dimethylmorpholine-4-carbonyl)-7-methoxy-4,5-dihydrobenzo[g]indazol-8-yl]pyridine-3-carboxamide (33 mg, 54.41 μmol, 1 eq) and DDQ (49.41 mg, 217.64 μmol, 4 eq) in dioxane (2 mL) was degassed and purged with N₂ for 3 times, and then the mixture was stirred at 80° C. for 16 h under N₂ atmosphere. The product was poured into ice-water (20 mL) and quenched with sat. Na₂SO₃ (10 mL). The aqueous phase was extracted with ethyl acetate (20 mL*3) and the combined organic phases washed with brine (50 mL), and dried over anhydrous Na₂SO₄. The mixture was filtered and the filtrate was concentrated in vacuo to provide a residue that was purified by prep-HPLC (column: Phenomenex Luna C18 150*25 mm*10 um; mobile phase: [water (TFA)-ACN]; gradient: 42%-72% B). Compound 5-[1-(3, 5-dichlorophenyl) -3-(3, 3-dimethylmorpholine-4-carbonyl)-7-methoxy-benzo[g]indazol-8-yl]pyridine-3-carboxamide (15 mg, 20.88 μmol, 38% yield, TFA) was obtained as a white solid. LCMS (ESI): m/z [M+H] calcd for C31H28Cl2N5O4: 604.14; found: 604.1. 1H NMR (400 MHz, METHANOL-d4) δ=8.87 (br s, 1H), 8.62 (br s, 1H), 8.29 (s, 1H), 7.83 (d, J=8.9 Hz, 1H), 7.70 (d, J=1.8 Hz, 2H), 7.69-7.60 (m, 3H), 7.53 (s, 1H), 3.91 (s, 3H), 3.74 (s, 4H), 3.47 (s, 2H), 1.51 (s, 6H). FIG. 59 shows the nuclear magnetic resonance of Compound 6-05.

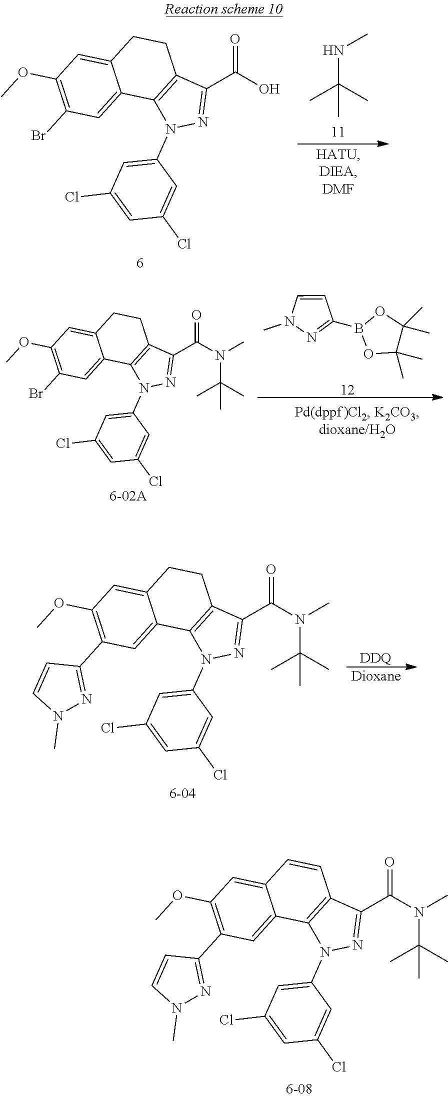

### Synthesis of 8-bromo-N-tert-butyl-1-(3,5-dichlorophenyl)-7-methoxy-N-methyl-4,5-dihydrobenzo [g]indazole-3-carb oxamide

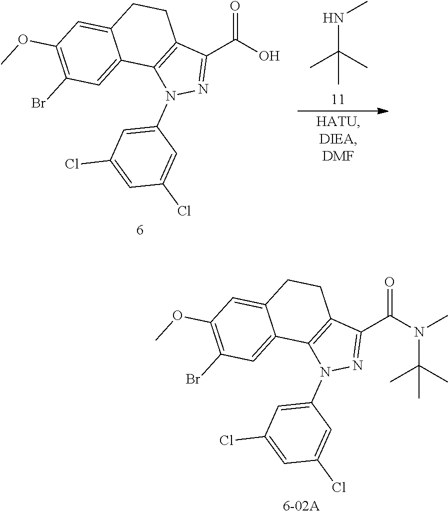

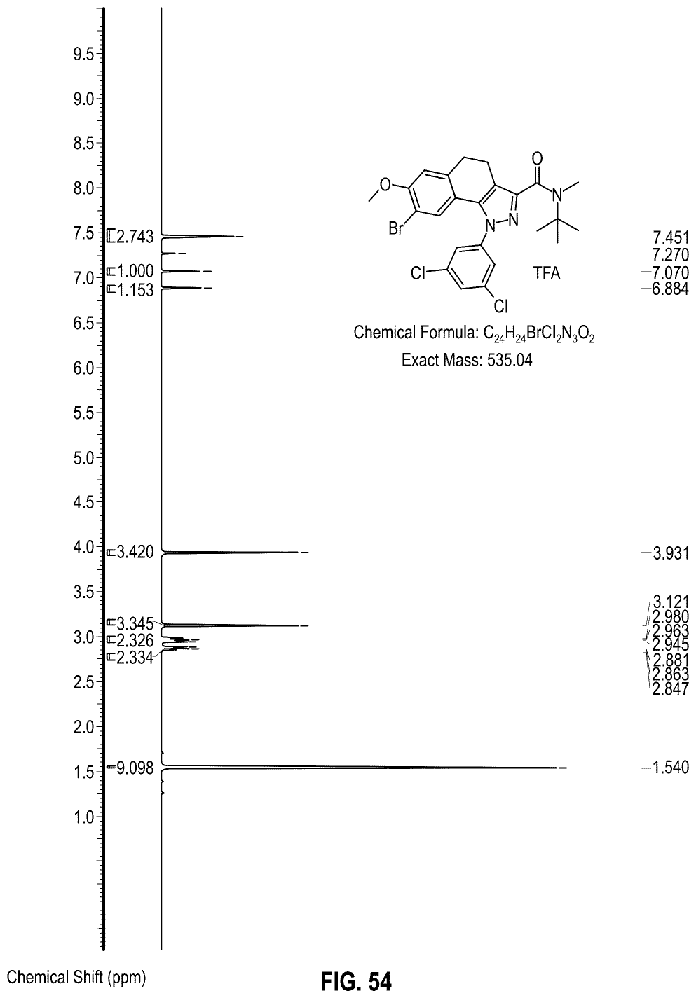

To a solution of 8-bromo-1-(3, 5-dichlorophenyl)-7-methoxy-4, 5-dihydrobenzo[g]indazole-3-carboxylic acid (1 g, 2.14 mmol, 1 eq) in DMF (10 mL) was added HATU (1.22 g, 3.20 mmol, 1.5 eq) and DIEA (828.25 mg, 6.41 mmol, 1.12 mL, 3 eq) at 25° C. After addition, the mixture was stirred at 25° C. for 0.5 h, and then N-2-dimethylpropan-2-amine (186.19 mg, 2.14 mmol, 256.11 μL, 1 eq) was added. The resulting mixture was stirred at 25° C. for 2 h then poured into ice-water (30 mL) and stirred for 3 min. The aqueous phase was extracted with ethyl acetate (20 mL*3). The combined organic phases were washed with brine (50 mL), dried over anhydrous Na₂SO₄, and filtered. The filtrate was concentrated in vacuo to provide a residue that was purified by flash silica gel chromatography (ISCO®; 20 g SepaFlash® Silica Flash Column, Eluent of 0˜30% Ethyl acetate/Petroleum ether gradient @60 mL/min) to get the crude product. The crude product was purified by prep-HPLC (column: Phenomenex Luna C18 150*25 mm*10 um; mobile phase: [water (TFA) -ACN]; gradient: 75%-100% B over 9 min). Compound 8-bromo-N-tert-butyl-1-(3, 5-dichlorophenyl)-7-methoxy-N-methyl-4, 5-dihydrobenzo[g]indazole-3-carboxamide (1.1 g, 1.69 mmol, 79% yield, TFA) was obtained as a white solid. LCMS (ESI): m/z [M+H] calcd for C₂₄H₂₅BrCl₂N₃O₂: 536.04; found: 535.9. ¹H NMR (400 MHz, CHLOROFORM-d) δ=7.45 (s, 3H), 7.07 (s, 1H), 6.88 (s, 1H), 3.93 (s, 3H), 3.12 (s, 3H), 3.00-2.93 (m, 2H), 2.91-2.83 (m, 2H), 1.54 (s, 9H). FIG. 54 shows the nuclear magnetic resonance of Compound 6-02A.

### Synthesis of N-tert-butyl-1-(3,5-dichlorophenyl)-7-methoxy-N-methyl-8-(1-methylpyrazol-3-yl)-4,5-dihydrobenzo[g]indazole-3-carboxamide

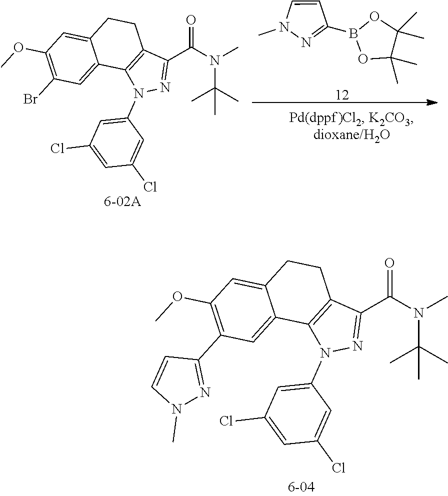

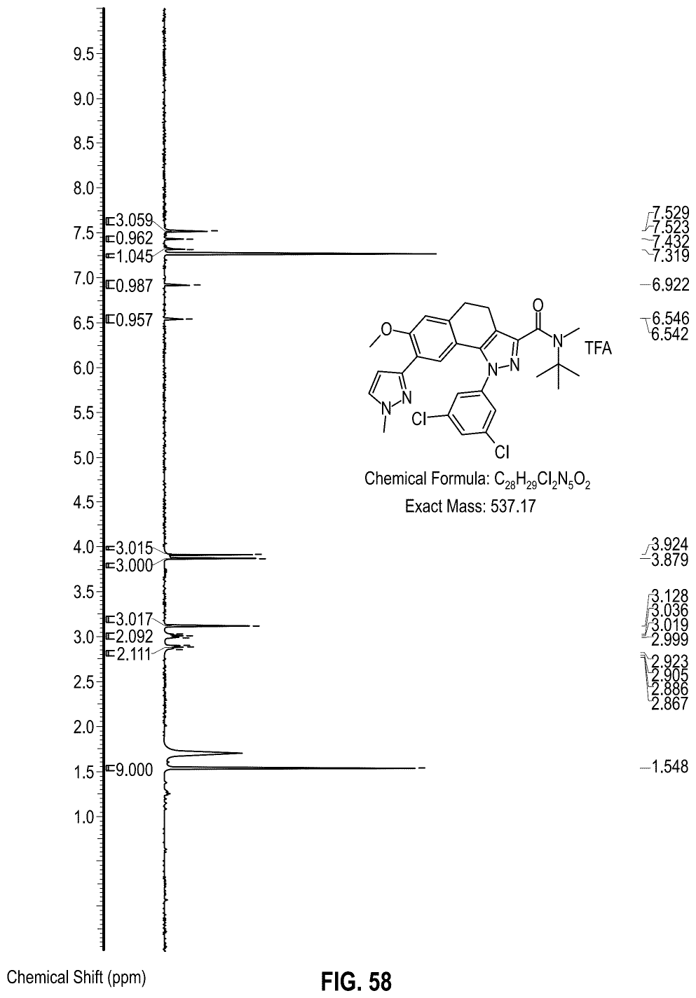

A mixture of 1-methyl-3-(4,4,5,5-tetramethyl-1,3,2-dioxaborolan-2-yl)pyrazole (63.90 mg, 307.11 μmol, 1.1 eq), 8-bromo-N-tert-butyl-1-(3,5-dichlorophenyl)-7-methoxy-N-methyl-4,5-dihydrobenzo[g]indazole-3-carboxamide (150 mg, 279.19 μmol, 1 eq), K₂CO₃ (77.17 mg, 558.37 μmol, 2 eq) and Pd(dppf)Cl₂ (20.43 mg, 27.92 μmol, 0.1 eq) in dioxane (3 mL) and H₂O (0.3 mL) was degassed and purged with N₂ 3 times, then heated to 80° C. for 16 h under N₂. The mixture was cooled and diluted with EtOAc (20 mL) and then filtered through a celite pad. The filtrate was concentrated to provide a residue that was purified by flash silica gel chromatography (ISCO®; 10 g SepaFlash® Silica Flash Column, Eluent of 0˜30% Ethyl acetate/Petroleum ether gradient @40 mL/min). Compound N-tert-butyl-1-(3,5-dichlorophenyl)-7-methoxy-N-methyl-8-(1-methylpyrazol-3-yl)-4,5-dihydrobenzo[g]indazole-3-carboxamide (0.1 g, 185.71 μmol, 67% yield) was obtained as a colorless oil. LCMS (ESI): m/z [M+H] calcd for C₂₈H₃₀Cl₂N₅O₂: 538.17; found: 538.3. FIG. 58 shows the nuclear magnetic resonance of Compound 6-04.

### Synthesis of N-tert-butyl-1-(3,5-dichlorophenyl)-7-methoxy-N-methyl-8-(1-methylpyrazol-3-yl)benzo[g]indazole-3-carboxamide

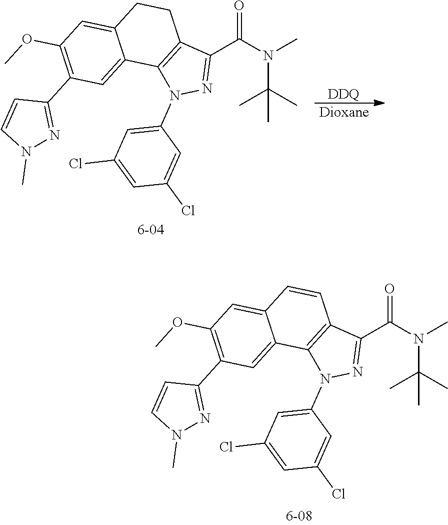

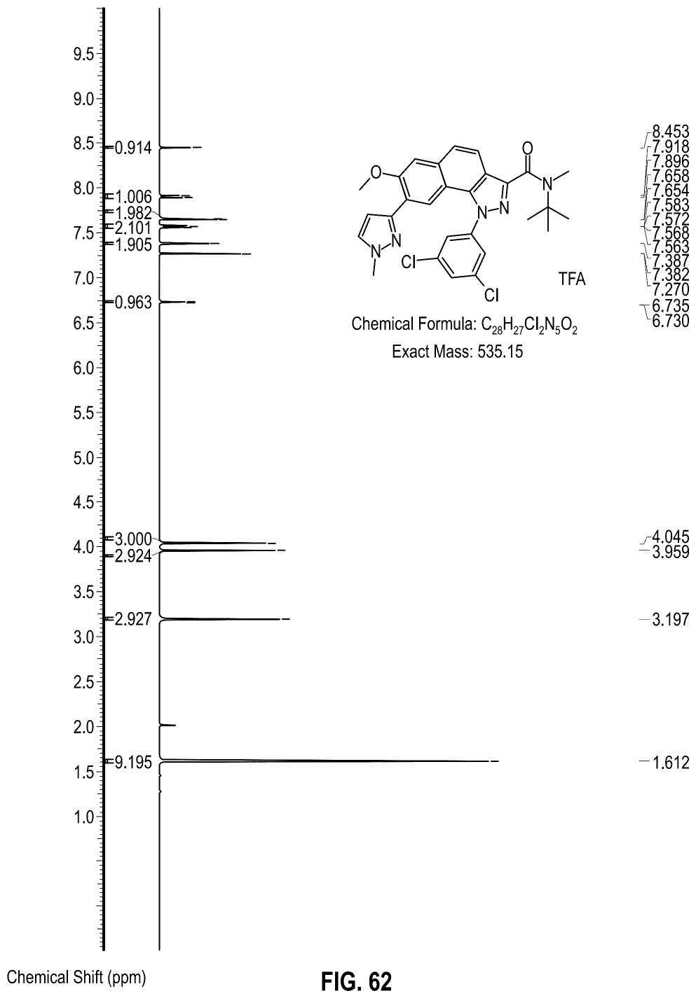

A mixture of N-tert-butyl-1-(3,5-dichlorophenyl)-7-methoxy-N-methyl-8-(1-methylpyrazol-3-yl)-4,5-dihydrobenzo[g]indazole-3-carboxamide (0.1 g, 185.71 μmol, 1 eq) and DDQ (168.63 mg, 742.85 μmol, 4 eq) in dioxane (2 mL) was degassed and purged with N₂ 3 times, and then the mixture was stirred at 80° C. for 16 h under N₂ atmosphere. The reaction was quenched with sat. Na₂SO₃ (5 mL) and the mixture was extracted with EtOAc (10 ml*2). The combined organic phases were washed with brine (10 ml) and dried over Na₂SO₄. The mixture was filtered and the filtrate was concentrated to provide a residue that was purified by prep-HPLC (column: Phenomenex Luna C18 150*25 mm*10 um; mobile phase: [water (TFA)-ACN]; gradient: 68%-98% B). Compound N-tert-butyl-1-(3,5-dichlorophenyl)-7-methoxy-N-methyl-8-(1-methylpyrazol-3-yl)benzo[g]indazole-3-carboxamide (25 mg, 38.43 μmol, 21% yield, TFA) was obtained as a white solid. LCMS (ESI): m/z [M+H] calcd for C₂₈H₂₈Cl₂N₅O₂: 536.15; found: 536.3. ¹H NMR (400 MHz, CHLOROFORM-d) δ=8.45 (s, 1H), 7.91 (d, J=8.8 Hz, 1H), 7.66 (d, J=1.9 Hz, 2H), 7.60-7.55 (m, 2H), 7.39-7.37 (m, 2H), 6.73 (d, J=2.1 Hz, 1H), 4.04 (s, 3H), 3.96 (s, 3H), 3.20 (s, 3H), 1.61 (s, 9H). FIG. 62 shows the nuclear magnetic resonance of Compound 6-08.

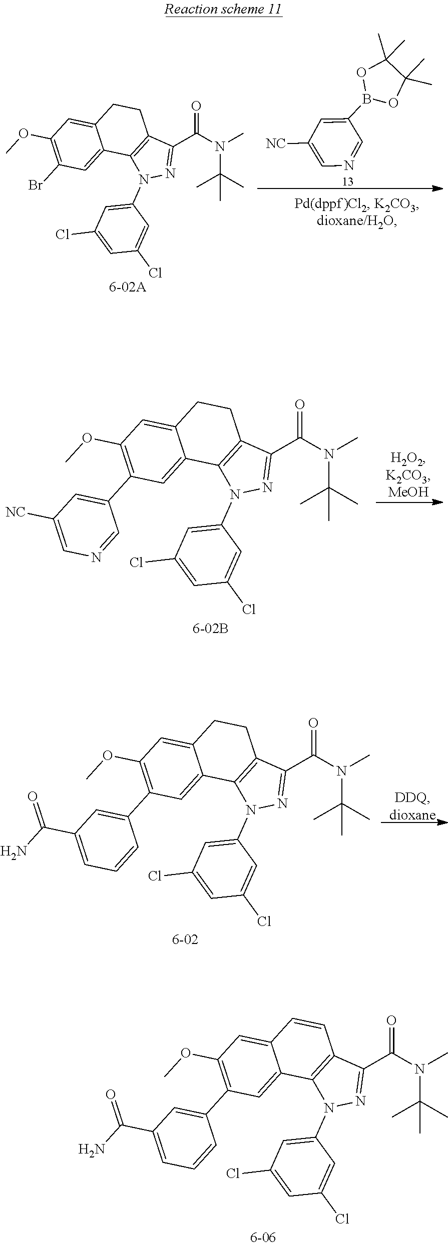

### Synthesis of N-tert-butyl-8-(5-cyano-3-pyridyl)-1-(3,5-dichlorophenyl)-7-methoxy-N-methyl-4,5-dihydrobenzo[g]indazole-3-carboxamide

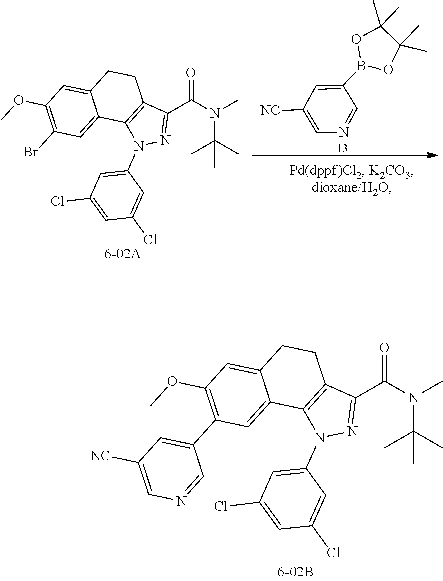 [SMILES: COc1cc2c(cc1Br)-c1c(c(C(=O)N(C)C(C)(C)C)nn1-c1cc(Cl)cc(Cl)c1)CC2 ; CC1(C)OB(c2cncc(C#N)c2)OC1(C)C ; COc1cc2c(cc1-c1cncc(C#N)c1)-c1c(c(C(=O)N(C)C(C)(C)C)nn1-c1cc(Cl)cc(Cl)c1)CC2]

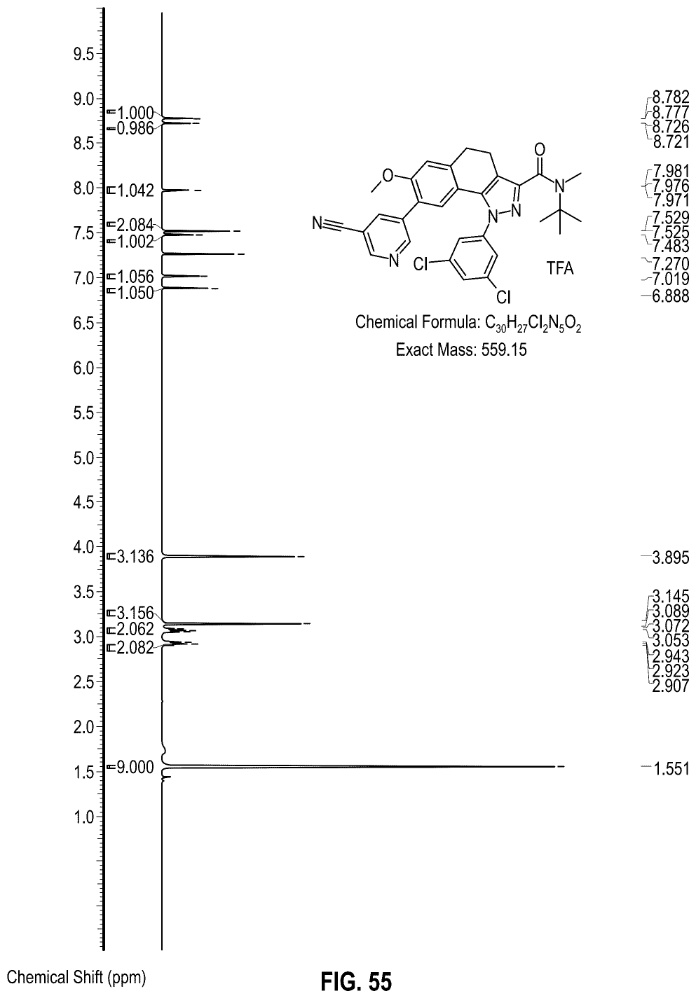

A mixture of 8-bromo-N-tert-butyl-1-(3,5-dichlorophenyl)-7-methoxy-N-methyl-4,5-dihydrobenzo[g]indazole-3-carboxamide (0.5 g, 930.62 μmol, 1 eq), 5-(4,4,5,5-tetramethyl-1,3,2-dioxaborolan-2-yl)pyridine-3-carbonitrile (235.52 mg, 1.02 mmol, 1.1 eq), K₂CO₃ (257.24 mg, 1.86 mmol, 2 eq) and Pd(dppf)Cl₂ (136.19 mg, 186.12 μmol, 0.2 eq) in dioxane (5 mL) and H₂O (0.5 mL) was degassed and purged with N₂ 3 times, then heated at 80° C. for 16 h under N₂. The mixture was diluted with EtOAc (40 mL) and then filtered. The filtrate was concentrated to provide a residue that was purified by flash silica gel chromatography (ISCO®; 12 g SepaFlash® Silica Flash Column, Eluent of 0˜40% Ethyl acetate/Petroleum ether gradient @40 mL/min).) to get the crude product. The crude product was further purified by prep-HPLC (column: Phenomenex Luna C18 150*25 mm*10 um; mobile phase: [water (TFA)-ACN]; gradient: 70%-100% B over 9 min). Compound N-tert-butyl-8-(5-cyano-3-pyridyl)-1-(3,5-dichlorophenyl)-7-methoxy-N-methyl-4,5-dihydrobenzo[g]indazole-3-carboxamide (390 mg, 578.21 μmol, 62% yield, TFA) was obtained as a white solid. LCMS (ESI): m/z [M+H] calcd for C30H28Cl2N5O2: 560.15; found: 560.2. ¹H NMR (400 MHz, CHLOROFORM-d) δ=8.78 (d, J=1.8 Hz, 1H), 8.72 (d, J=2.0 Hz, 1H), 7.98 (t, J=2.0 Hz, 1H), 7.53 (d, J=1.8 Hz, 2H), 7.48 (d, J=1.8 Hz, 1H), 7.02 (s, 1H), 6.89 (s, 1H), 3.90 (s, 3H), 3.14 (s, 3H), 3.10-3.04 (m, 2H), 2.96-2.89 (m, 2H), 1.55 (s, 9H). FIG. 55 shows the nuclear magnetic resonance of Compound 6-02B.

### Synthesis of N-tert-butyl-8-(5-carbamoyl-3-pyridyl)-1-(3,5-dichlorophenyl)-7-methoxy-N-methyl-4,5-dihydrobenzo[g]indazole-3-carboxamide

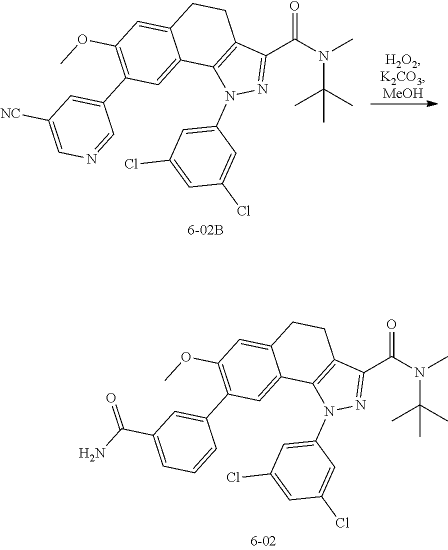 [SMILES: COc1cc2c(cc1-c1cncc(C#N)c1)-c1c(c(C(=O)N(C)C(C)(C)C)nn1-c1cc(Cl)cc(Cl)c1)CC2 ; COc1cc2c(cc1-c1cccc(C(N)=O)c1)-c1c(c(C(=O)N(C)C(C)(C)C)nn1-c1cc(Cl)cc(Cl)c1)CC2]

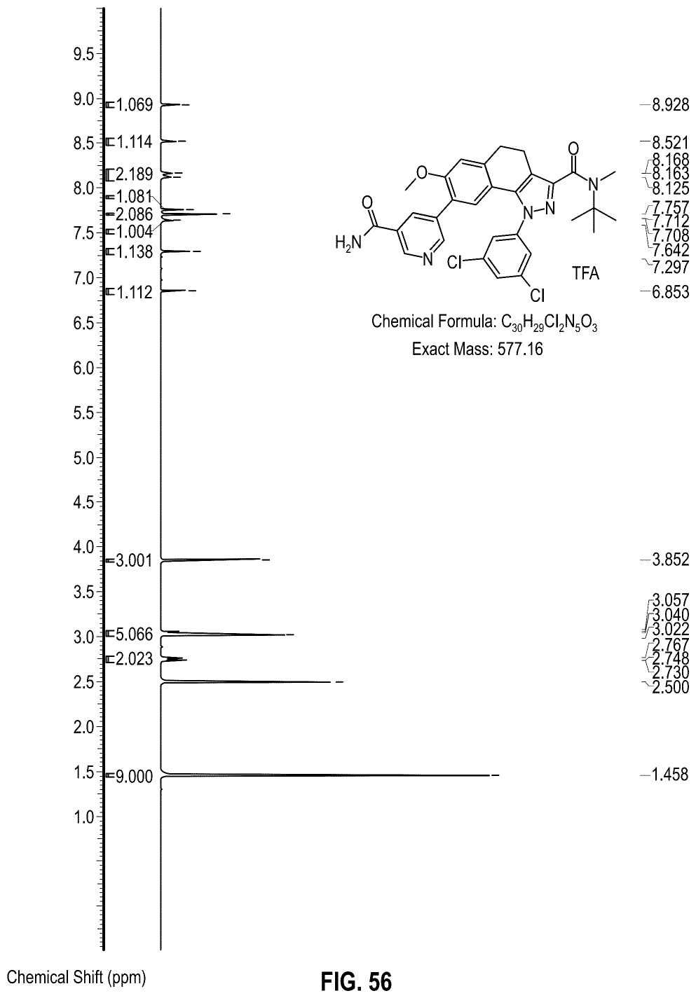

To a solution of N-tert-butyl-8-(5-cyano-3-pyridyl)-1-(3,5-dichlorophenyl)-7-methoxy-N-methyl-4,5-dihydrobenzo[g]indazole-3-carboxamide (0.3 g, 535.26 μmol, 1 eq) in MeOH (5 mL) was added K₂CO₃ (3 M, 356.84 μL, 2 eq) and H₂O₂ (0.45 g, 3.97 mmol, 381.36 μL, 30% purity, 7.41 eq). The mixture was stirred at 25° C. for 16 h then quenched with sat. Na2SO3 (5 mL). The aqueous phase was extracted with ethyl acetate (10 mL*3). The combined organic phases were washed with brine (20 mL) and dried over anhydrous Na2SO4, The mixture was filtered and the filtrate was concentrated to provide a residue that was purified by prep-HPLC (column: Phenomenex Luna C18 150*40 mm*15 um; mobile phase: [water (TFA) -ACN]; gradient: 45%-75% B over 15 min). Compound N-tert-butyl-8-(5-carbamoyl-3-pyridyl) -1-(3, 5-dichlorophenyl)-7-methoxy-N-methyl-4, 5-dihydrobenzo[g]indazole-3-carboxamide (150 mg, 216.60 μmol, 40% yield, TFA) was obtained as a white solid. LCMS (ESI): m/z [M+H] calcd for C30H30Cl2N5O3: 578.16; found: 578.2. ¹H NMR (400 MHz, DMSO-d₆) δ=8.93 (s, 1H), 8.52 (br s, 1H), 8.21-8.08 (m, 2H), 7.76 (s, 1H), 7.71 (d, J=1.8 Hz, 2H), 7.64 (br s, 1H), 7.30 (s, 1H), 6.85 (s, 1H), 3.85 (s, 3H), 3.07-3.00 (m, 5H), 2.75 (br t, J=7.4 Hz, 2H), 1.46 (s, 9H). FIG. 56 shows the nuclear magnetic resonance of Compound 6-02.

### Synthesis of N-tert-butyl-8-(5-carbamoyl-3-pyridyl)-1-(3,5-dichlorophenyl)-7-methoxy-N-methyl-benzo[g]indazole-3-carboxamide

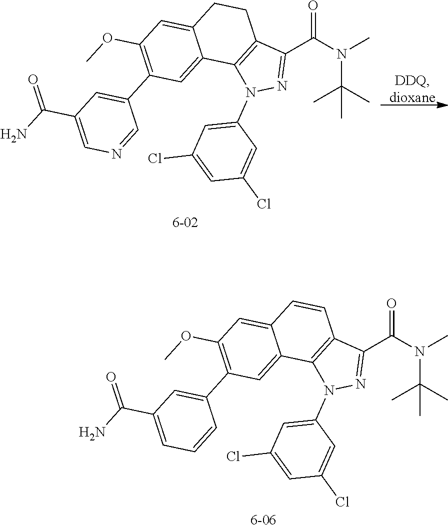

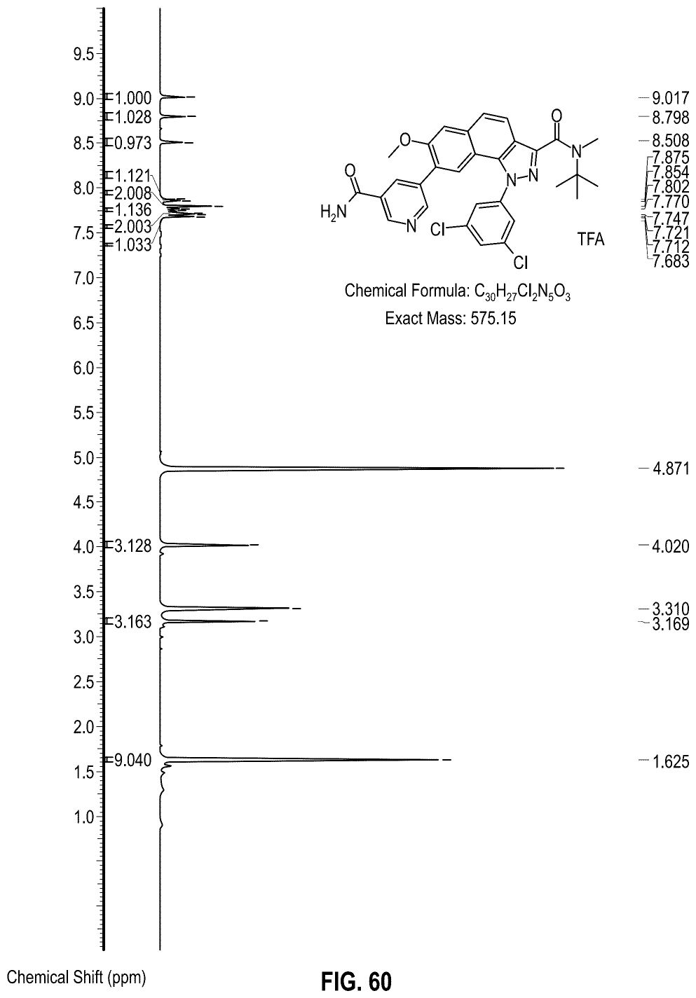

A mixture of N-tert-butyl-8-(5-carbamoyl-3-pyridyl)-1-(3,5-dichlorophenyl)-7-methoxy-N-methyl-4, 5-dihydrobenzo[g]indazole-3-carboxamide (0.1 g, 172.86 μmol, 1 eq) and DDQ (156.96 mg, 691.46 μmol, 4 eq) in dioxane (2 mL) was stirred at 80° C. for 5 h under N₂ atmosphere. The residue was then poured into ice-water (20 mL) and quenched with sat. Na2SO3 (10 mL). The aqueous phase was extracted with ethyl acetate (10 mL*3). The combined organic phases were washed with brine (20 mL) and dried over anhydrous Na₂SO₄. The mixture was filtered and the filtrate was concentrated in vacuo to provide a residue that was purified by prep-HPLC (column: Phenomenex Luna C18 150*25 mm*10 um; mobile phase: [water (TFA)-ACN]; gradient: 50%-80% B over min). Compound N-tert-butyl-8-(5-carbamoyl-3-pyridyl)-1-(3,5-dichlorophenyl)-7-methoxy-N-methyl-benzo[g]indazole-3-carboxamide (10 mg, 14.48 μmol, 8% yield, TFA) was obtained as a white solid. LCMS (ESI): m/z [M+H] calcd for C30H28Cl2N5O3: 576.15; found: 576.2. ¹H NMR (400 MHz, METHANOL-d₄) δ=9.02 (s, 1H), 8.80 (s, 1H), 8.51 (br s, 1H), 7.86 (br d, J=8.6 Hz, 1H), 7.80 (s, 2H), 7.76 (br d, J=9.2 Hz, 1H), 7.72 (br d, J=3.8 Hz, 2H), 7.68 (s, 1H), 4.02 (s, 3H), 3.17 (s, 3H), 1.63 (s, 9H). FIG. 60 shows the nuclear magnetic resonance of Compound 6-06.

### Example 8

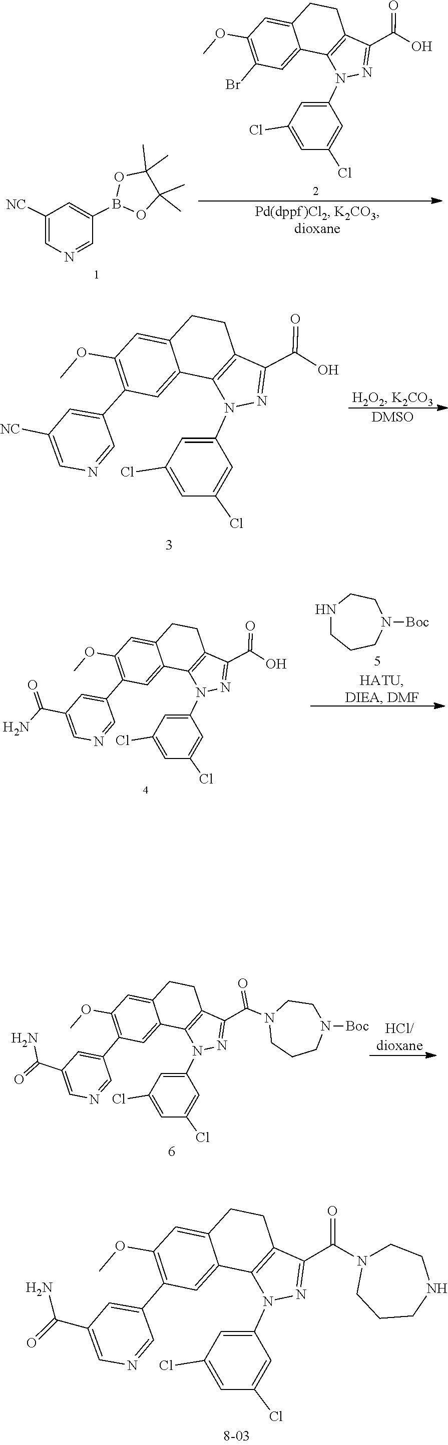

### Synthesis of 8-(5-cyanopyridin-3-yl)-1-(3,5-dichlorophenyl)-7-methoxy-4,5-dihydro-1H-benzo[g]indazole-3-carboxylic acid

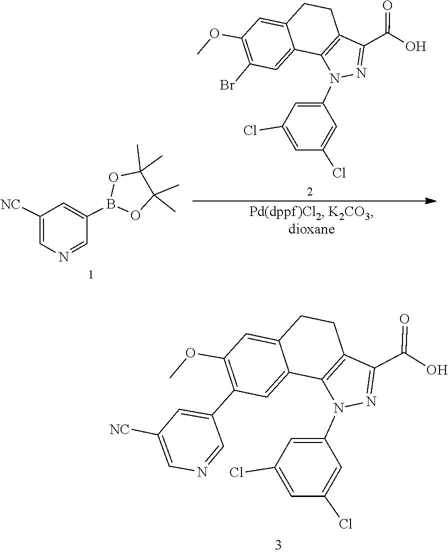 [SMILES: CC1(C)OB(c2cncc(C#N)c2)OC1(C)C ; COc1cc2c(cc1Br)-c1c(c(C(=O)O)nn1-c1cc(Cl)cc(Cl)c1)CC2 ; COc1cc2c(cc1-c1cncc(C#N)c1)-c1c(c(C(=O)O)nn1-c1cc(Cl)cc(Cl)c1)CC2]

To a mixture of 5-(4,4,5,5-tetramethyl-1,3,2-dioxaborolan-2-yl)nicotinonitrile (1.46 g, 6.36 mmol, 1.2 eq) in dioxane (20 mL) and H₂O (4 mL) was added K₂CO₃ (2.20 g, 15.89 mmol, 3 eq), 8-bromo-1-(3,5-dichlorophenyl)-7-methoxy-4,5-dihydro-1H-benzo[g]indazole-3-carboxylic acid (2.48 g, 5.30 mmol, 1 eq) and Pd(dppf)Cl₂ (775.27 mg, 1.06 mmol, 0.2 eq). The mixture was stirred at 60° C. for 16 h under N₂ atmosphere. The reaction mixture was poured into H₂O (100 mL) and ethyl acetate (100 mL), and extracted with ethyl acetate (100 mL*3). The combined organic phase was dried over anhydrous Na2SO4 and filtered. The filtrate was concentrated under vacuum to give a residue. The crude product was triturated with MTBE (20 mL) at 15° C. for 15 min to afford 8-(5-cyanopyridin-3-yl)-1-(3,5-dichlorophenyl)-7-methoxy-4,5-dihydro-1H-benzo[g]indazole-3-carboxylic acid (4 g, crude) as a black solid. ¹H NMR (400 MHz, DMSO-d6) δ=8.96-8.87 (m, 1H), 8.70 (s, 1H), 8.15 (s, 1H), 7.68 (s, 1H), 7.63 (s, 2H), 7.26 (s, 1H), 6.90 (br s, 1H), 3.84 (s, 3H), 2.99-2.89 (m, 4H).

### Synthesis of 8-(5-carbamoyl-3-pyridyl)-1-(3,5-dichlorophenyl)-7-methoxy-4,5-dihydrobenzo[g]indazole-3-carboxylic acid

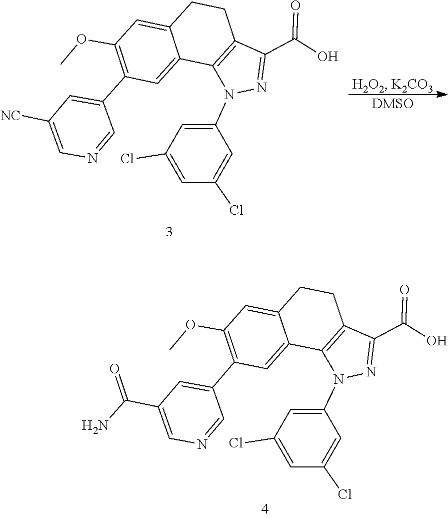 [SMILES: COc1cc2c(cc1-c1cncc(C#N)c1)-c1c(c(C(=O)O)nn1-c1cc(Cl)cc(Cl)c1)CC2 ; COc1cc2c(cc1-c1cncc(C(N)=O)c1)-c1c(c(C(=O)O)nn1-c1cc(Cl)cc(Cl)c1)CC2]

To a mixture of 8-(5-cyanopyridin-3-yl)-1-(3,5-dichlorophenyl)-7-methoxy-4,5-dihydro-1H-benzo[g]indazole-3-carboxylic acid (200 mg, 407.06 μmol, 1 eq) in DMSO (2 mL) was added K₂CO₃ (112.52 mg, 814.13 μmol, 2 eq) and H₂O₂ (461.54 mg, 4.07 mmol, 391.13 μL, 30% purity, 10 eq). The mixture was stirred at 15° C. for 15 min. Saturated aqueous Na₂S₂O₃ (10 mL) was added, and the mixture was stirred at 15° C. for 1 h. The pH of the reaction mixture was adjusted to 7 with 1 M HCl aqueous solution. The mixture was extracted with ethyl acetate (10 mL*3).The combined organic phase was dried over anhydrous Na₂SO₄ and filtered. The mixture was concentrated under vacuum to give a residue. The residue was purified by prep-HPLC (column: Phenomenex Luna C18 150*25 mm*10 um;mobile phase: [water(TFA)-ACN];gradient:300%-60% B over 9 min). Compound 8-bromo-1-(3,5-dichlorophenyl)-7-methoxy-4,5-dihydro-1H-benzo[g]indazole-3-carboxylic acid (30 mg, 58.90 μmol, 14% yield) was obtained as an off-white solid.

### Synthesis of tert-butyl 4-[8-(5-carbamoyl-3-pyridyl)-1-(3,5-dichlorophenyl)-7-methoxy-4,5-dihydrobenzo[g]indazole-3-carbonyl]-1,4-diazepane-1-carboxylate

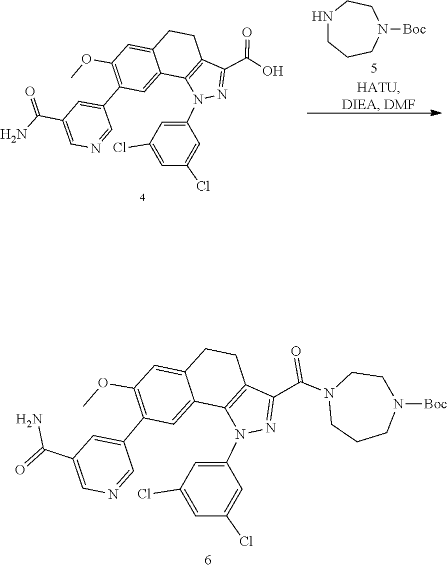 [SMILES: COc1cc2c(cc1-c1cncc(C(N)=O)c1)-c1c(c(C(=O)O)nn1-c1cc(Cl)cc(Cl)c1)CC2 ; CC(C)(C)OC(=O)N1CCCNCC1 ; COc1cc2c(cc1-c1cncc(C(N)=O)c1)-c1c(c(C(=O)N3CCCN(C(=O)OC(C)(C)C)CC3)nn1-c1cc(Cl)cc(Cl)c1)CC2]

To a mixture of 8-bromo-1-(3,5-dichlorophenyl)-7-methoxy-4,5-dihydro-1H-benzo[g]indazole-3-carboxylic acid (30 mg, 58.90 mol, 1 eq) in DMF (2 mL) was added DIEA (22.84 mg, 176.70 μmol, 30.78 μL, 3 eq) and HATU (33.59 mg, 88.35 μmol, 1.5 eq).The mixture was stirred at 25° C. for 0.5 h. Then tert-butyl 1,4-diazepane-1-carboxylate (21.23 mg, 106.02 μmol, 20.90 μL, 1.8 eq) was added to the reaction mixture. The mixture was stirred at 25° C. for 16 h. The mixture was poured into H₂O(2 mL). There was precipitate formed in the reaction mixture, the mixture was filtered and the filter cake was collected and dried in vacuum to give a residue. Compound tert-butyl 4-[8-(5-carbamoyl-3-pyridyl)-1-(3,5-dichlorophenyl)-7-methoxy-4,5-dihydrobenzo[g]indazole-3-carbonyl]-1,4-diazepane-1-carboxylate (30 mg, crude) was obtained as a black solid.

### Synthesis of 5-[3-(1,4-diazepane-1-carbonyl)-1-(3,5-dichlorophenyl)-7-methoxy-4,5-dihydrobenzo[g]indazol-8-yl]pyridine-3-carboxamide

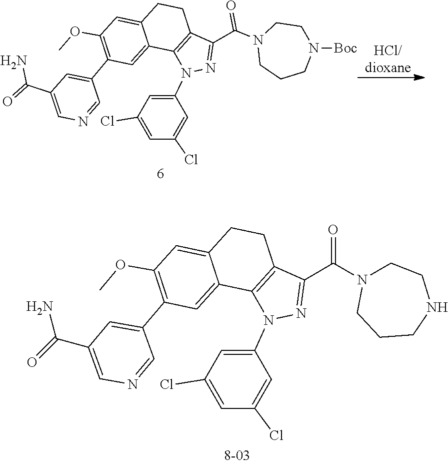 [SMILES: COc1cc2c(cc1-c1cncc(C(N)=O)c1)-c1c(c(C(=O)N3CCCN(C(=O)OC(C)(C)C)CC3)nn1-c1cc(Cl)cc(Cl)c1)CC2 ; COc1cc2c(cc1-c1cncc(C(N)=O)c1)-c1c(c(C(=O)N3CCCNCC3)nn1-c1cc(Cl)cc(Cl)c1)CC2]

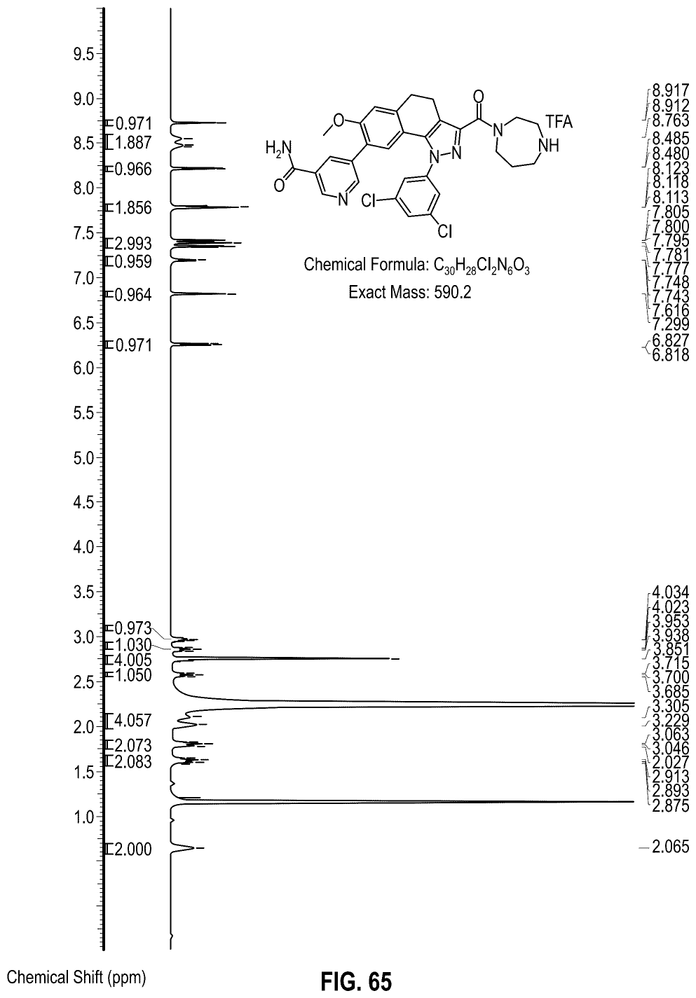

A mixture of tert-butyl 4-[8-(5-carbamoyl-3-pyridyl)-1-(3,5-dichlorophenyl)-7-methoxy-4,5-dihydrobenzo[g]indazole-3-carbonyl]-1,4-diazepane-1-carboxylate (30 mg, 43.38 μmol, 1 eq) in 4 N HCl/EtOAc (1 mL) was stirred at 15° C. for 1 h. The mixture was concentrated under vacuum to give a residue. The residue was purified by prep-HPLC (column: Phenomenex Luna C18 150*25 mm*10 um;mobile phase: [water (TFA) -ACN];gradient: 18%-48% B over 9 min) followed by lyophilization to afford 5-[3-(1,4-diazepane-1-carbonyl)-1-(3,5-dichlorophenyl)-7-methoxy-4,5-dihydrobenzo[g]indazol-8-yl]pyridine-3-carboxamide (4.29 mg, 5.84 μmol, 13% yield, 96% purity, TFA) as a brown gum. LCMS (ESI): m/z [M+H] calcd for C₃₂H₂₉N₆O₅Cl₂F₃: 591.16; found: 591.2. 1H NMR (400 MHz, DMSO-d6) δ=8.91 (d, J=1.9 Hz, 1H), 8.81-8.66 (m, 2H), 8.48 (d, J=2.0 Hz, 1H), 8.12 (t, J=1.9 Hz, 2H), 7.82-7.73 (m, 3H), 7.62 (s, 1H), 7.30 (s, 1H), 6.82 (d, J=3.6 Hz, 1H), 4.03 (br d, J=4.5 Hz, 1H), 3.94 (br t, J=6.0 Hz, 1H), 3.85 (s, 3H), 3.70 (br t, J=6.0 Hz, 1H), 3.33-3.19 (m, 4H), 3.08-3.00 (m, 2H), 2.88 (q, J=7.4 Hz, 2H), 2.06 (br d, J=0.8 Hz, 2H). FIG. 65 shows the nuclear magnetic resonance of Compound 8-03.

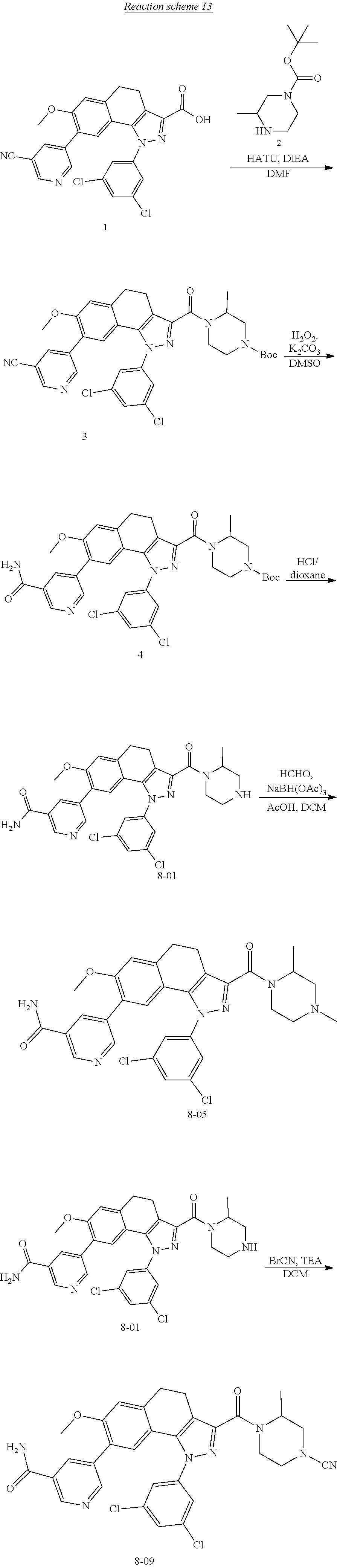

### Synthesis of tert-butyl 4-[8-(5-cyano-3-pyridyl)-1-(3,5-dichlorophenyl)-7-methoxy-4,5-dihydrobenzo[g]indazole-3-carbonyl]-3-methyl-piperazine-1-carboxylate

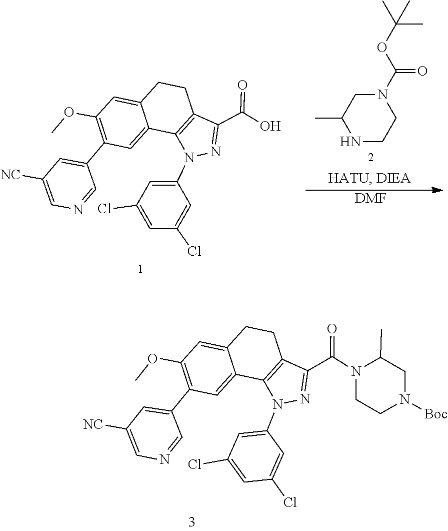

To a solution of 8-(5-cyano-3-pyridyl)-1-(3,5-dichlorophenyl)-7-methoxy-4,5-dihydrobenzo[g]indazole-3-carboxylic acid (250 mg, 508.83 μmol, 1 eq) in DMF (3 mL) was added HATU (290.21 mg, 763.24 μmol, 1.5 eq) and DIEA (197.29 mg, 1.53 mmol, 265.89 μL, 3 eq). The mixture stirred at 25° C. for 30 min. Then a mixture of tert-butyl 3-methylpiperazine-1-carboxylate (183.43 mg, 915.89 μmol, 1.8 eq) in DMF (1.5 mL) was added. The mixture stirred at 25° C. for 16 hr. The reaction mixture was diluted with water (10 mL) and filtered to give the filter cake. The filter cake was collected and dried in vacuum. Compound tert-butyl 4-[8-(5-cyano-3-pyridyl)-1-(3,5-dichlorophenyl)-7-methoxy-4,5-dihydrobenzo[g]indazole-3-carbonyl]-3-methyl-piperazine-1-carboxylate (200 mg, crude) was obtained as a brown solid. ¹H NMR (400 MHz, CHLOROFORM-d) δ=8.83-8.67 (m, 2H), 8.04-7.96 (m, 1H), 7.51 (s, 3H), 7.02 (s, 1H), 6.92-6.83 (m, 1H), 4.93 (br d, J=8.0 Hz, 1H), 4.60-4.38 (m, 1H), 3.90 (s, 4H), 3.21-3.03 (m, 3H), 3.00-2.95 (m, 3H), 2.89 (s, 2H), 1.49 (s, 9H), 1.32 (br d, J=4.4 Hz, 3H).

### Synthesis of tert-butyl 4-[8-(5-carbamoyl-3-pyridyl)-1-(3,5-dichlorophenyl)-7-methoxy-4,5-dihydrobenzo [g]indazole-3-carbonyl]-3-methyl-piperazine-1-carboxylate

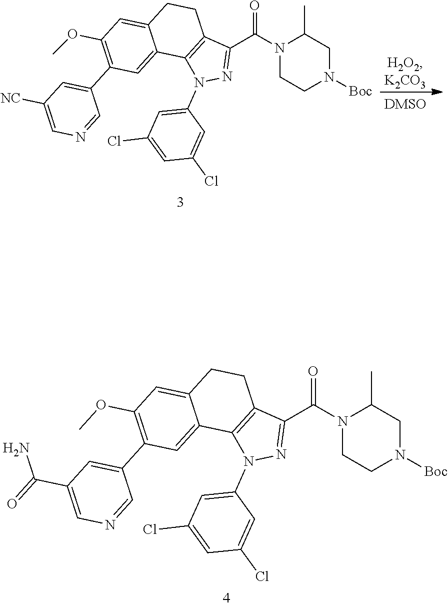 [SMILES: COc1cc2c(cc1-c1cncc(C#N)c1)-c1c(c(C(=O)N3CCN(C(=O)OC(C)(C)C)CC3C)nn1-c1cc(Cl)cc(Cl)c1)CC2 ; COc1cc2c(cc1-c1cncc(C(N)=O)c1)-c1c(c(C(=O)N3CCN(C(=O)OC(C)(C)C)CC3C)nn1-c1cc(Cl)cc(Cl)c1)CC2]

To a solution of tert-butyl 4-[8-(5-cyano-3-pyridyl)-1-(3,5-dichlorophenyl)-7-methoxy-4, 5-dihydrobenzo[g]indazole-3-carbonyl]-3-methyl-piperazine-1-carboxylate (180 mg, 267.23 μmol, 1 eq) in DMSO (2 mL) was added K₂CO₃ (3 M, 178.15 μL, 2 eq) and H₂O₂ (50 mg, 440.99 μmol, 42.37 μL, 30% purity, 1.65 eq). The mixture was stirred at 20-60° C. for 18 hr. The reaction mixture was diluted with saturated Na2SO3 aqueous solution (20 mL). The aqueous layer was extracted with ethyl acetate (20 mL*3). The combined organic layers were washed with brine (10 mL) and dried with anhydrous Na₂SO₄, filtered and concentrated in vacuum to give a residue. Compound tert-butyl 4-[8-(5-carbamoyl-3-pyridyl -1-(3,5-dichlorophenyl)-7-methoxy-4,5-dihydrobenzo[g]indazole-3-carbonyl]-3-methyl-piperazine-1-carboxylate (184 mg, 266.05 μmol, 99% yield) was obtained as a yellow oil.

### Synthesis of 5-[1-(3,5-dichlorophenyl)-7-methoxy-3-(2-methylpiperazine-1-carbonyl)-4,5-dihydrobenzo[g]indazol-8-yl]pyridine-3-carboxamide

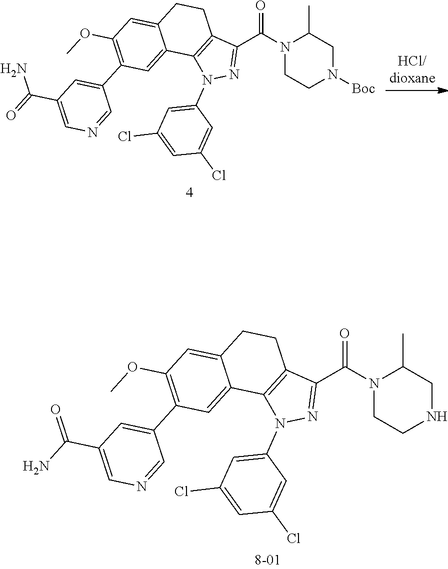 [SMILES: COc1cc2c(cc1-c1cncc(C(N)=O)c1)-c1c(c(C(=O)N3CCN(C(=O)OC(C)(C)C)CC3C)nn1-c1cc(Cl)cc(Cl)c1)CC2 ; COc1cc2c(cc1-c1cncc(C(N)=O)c1)-c1c(c(C(=O)N3CCNCC3C)nn1-c1cc(Cl)cc(Cl)c1)CC2]

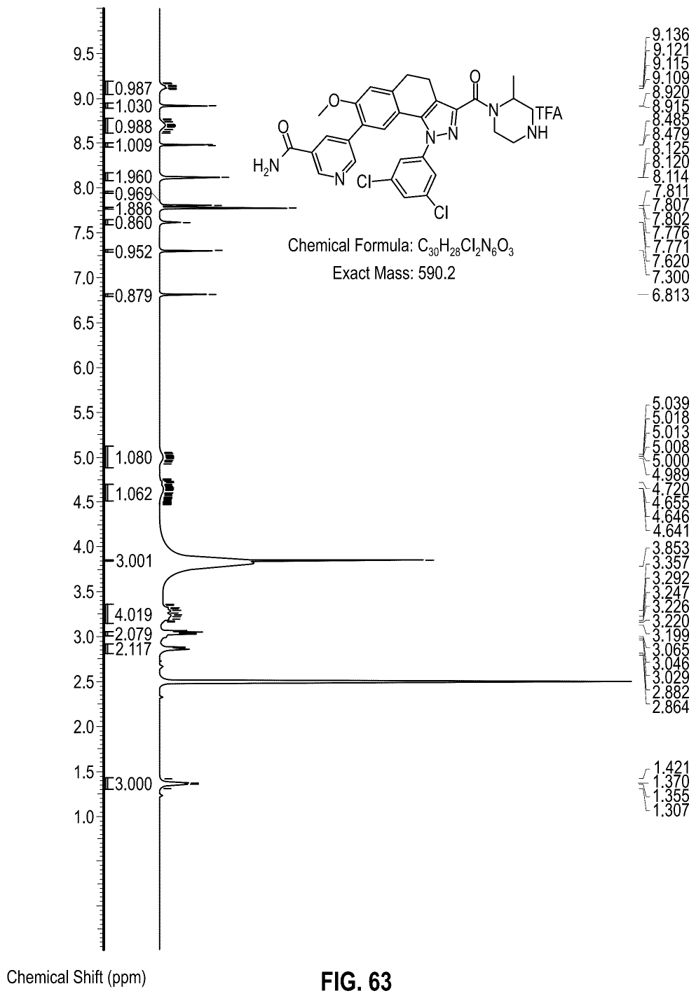

A mixture of tert-butyl 4-[8-(5-carbamoyl-3-pyridyl)-1-(3,5-dichlorophenyl)-7-methoxy-4,5-dihydrobenzo[g]indazole-3-carbonyl]-3-methyl-piperazine-1-carboxylate (130 mg, 187.97 μmol, 1 eq) in 4 N HCl/EtOAc (2 mL) was stirred at 15° C. for 1 h. The mixture was concentrated under vacuum to give a residue. The residue was purified by prep-HPLC (column: Phenomenex luna C18 150*25 mm*10 um;mobile phase: [water (TFA) -ACN];gradient: 20%-50% B over min) followed by lyophilization to afford 5-[1-(3,5-dichlorophenyl)-7-methoxy-3-(2-methylpiperazine-1-carbonyl)-4,5-dihydrobenzo[g]indazol-8-yl]pyridine-3-carboxamide (50 mg, 70.87 μmol, 38% yield, 100% purity, TFA) as an off-white solid. LCMS (ESI): m/z [M+H] calcd for C₃₂H₂₉N₆Cl₂O₅F₃: 591.16; found: 591.2 1H NMR (400 MHz, DMSO-d6) δ=9.19-9.05 (m, 1H), 8.92 (d, J=2.0 Hz, 1H), 8.78-8.61 (m, 1H), 8.48 (d, J=2.1 Hz, 1H), 8.17-8.08 (m, 2H), 7.82-7.79 (m, 1H), 7.77 (d, J=1.9 Hz, 2H), 7.62 (s, 1H), 7.30 (s, 1H), 6.81 (s, 1H), 5.13-4.89 (m, 1H), 4.71-4.51 (m, 1H), 3.85 (s, 3H), 3.37-3.15 (m, 4H), 3.04 (br d, J=7.0 Hz, 2H), 2.87 (br d, J=7.1 Hz, 2H), 1.36 (br d, J=5.9 Hz, 3H). FIG. 63 shows the nuclear magnetic resonance of Compound 8-01.

### Synthesis of 5-[1-(3,5-dichlorophenyl)-3-(2,4-dimethylpiperazine-1-carbonyl)-7-methoxy-4,5-dihydrobenzo[g]indazol-8-yl]pyridine-3-carboxamide

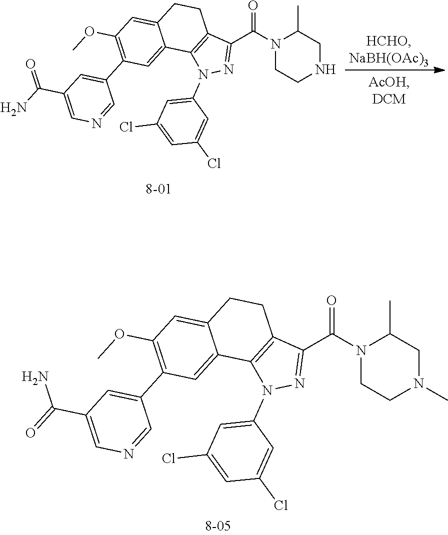 [SMILES: COc1cc2c(cc1-c1cncc(C(N)=O)c1)-c1c(c(C(=O)N3CCNCC3C)nn1-c1cc(Cl)cc(Cl)c1)CC2 ; COc1cc2c(cc1-c1cncc(C(N)=O)c1)-c1c(c(C(=O)N3CCN(C)CC3C)nn1-c1cc(Cl)cc(Cl)c1)CC2]

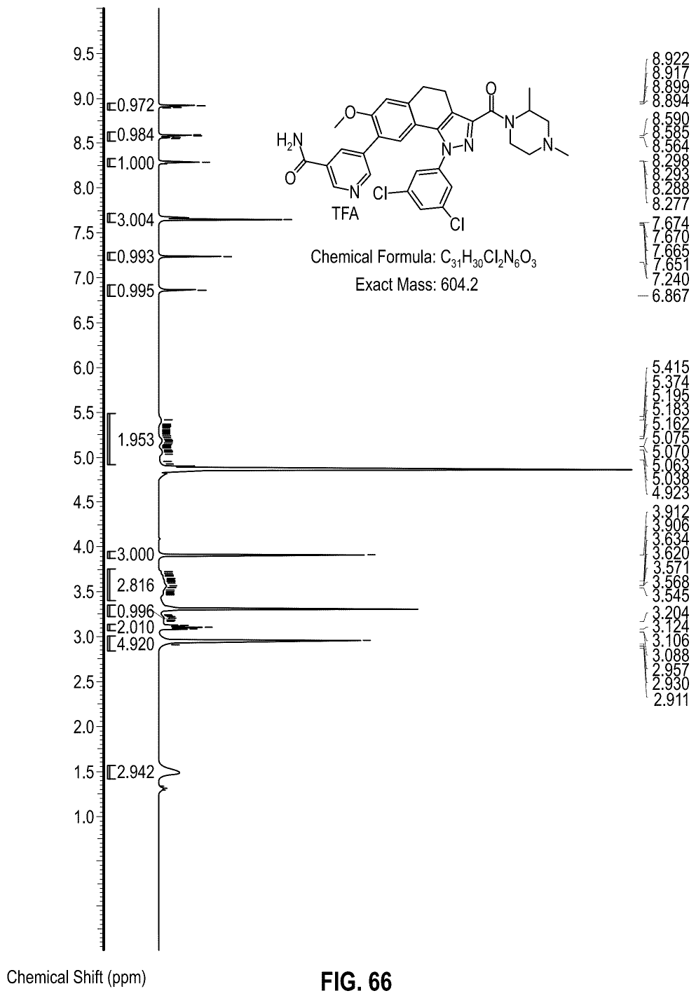

To a mixture of 5-[1-(3,5-dichlorophenyl)-7-methoxy-3-(2-methylpiperazine-1-carbonyl)-4,5-dihydrobenzo[g]indazol-8-yl]pyridine-3-carboxamide (40 mg, 67.63 μmol, 1 eq) in DCM (3 mL) was added AcOH (812.22 pg, 13.53 μmol, 7.74e-1 μL, 0.2 eq) and HCHO (6.59 mg, 81.15 μmol, 6.04 μL, 1.2 eq, 37% in H₂O). The mixture was stirred at 15° C. for 1 h, then NaBH(OAc)₃ (43.00 mg, 202.88 μmol, 3 eq) was added. The mixture was stirred at 15° C. for 16 h. The mixture was concentrated under vacuum to give a residue. The residue was purified by prep-HPLC (column: Phenomenex Luna C18 150*25 mm*10 um;mobile phase: [water (TFA) -ACN];gradient: 18%-48% B over 9 min) followed by lyophilization to afford 5-[1-(3,5-dichlorophenyl)-3-(2,4-dimethylpiperazine-1-carbonyl)-7-methoxy-4,5-dihydrobenzo[g]indazol-8-yl]pyridine-3-carboxamide (22.94 mg, 31.88 μmol, 47% yield, 100% purity, TFA) as an off-white solid.LCMS (ESI): m/z [M+H] calcd for C₃₃H₃₁N₆Cl₂O₅F₃: 605.18; found: 605.2. ¹H NMR (400 MHz, METHANOL-d4) δ=8.94-8.89 (m, 1H), 8.61-8.55 (m, 1H), 8.31-8.26 (m, 1H), 7.69-7.63 (m, 3H), 7.24 (s, 1H), 6.87 (s, 1H), 5.49-4.91 (m, 2H), 3.94-3.88 (m, 3H), 3.57 (br s, 3H), 3.25-3.15 (m, 1H), 3.14-3.06 (m, 2H), 3.03-2.87 (m, 5H), 1.56-1.41 (m, 3H). FIG. 66 shows the nuclear magnetic resonance of Compound 8-05.

### Synthesis of 5-[3-(4-cyano-2-methyl-piperazine-1-carbonyl)-1-(3,5-dichlorophenyl)-7-methoxy-4,5-dihydrobenzo[g]indazol-8-yl]pyridine-3-carboxamide

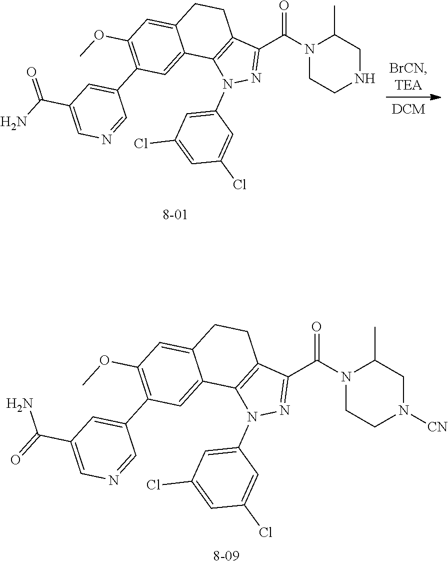 [SMILES: COc1cc2c(cc1-c1cncc(C(N)=O)c1)-c1c(c(C(=O)N3CCNCC3C)nn1-c1cc(Cl)cc(Cl)c1)CC2 ; COc1cc2c(cc1-c1cncc(C(N)=O)c1)-c1c(c(C(=O)N3CCN(C#N)CC3C)nn1-c1cc(Cl)cc(Cl)c1)CC2]

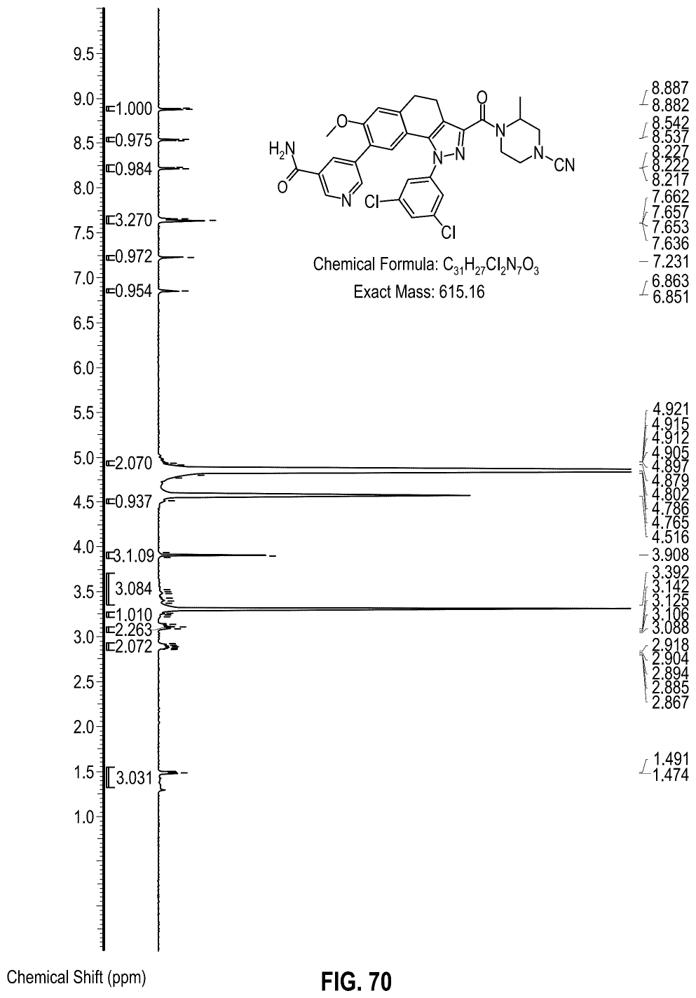

To a solution of 5-[1-(3,5-dichlorophenyl)-7-methoxy-3-(2-methylpiperazine-1-carbonyl) -4,5-dihydrobenzo[g]indazol-8-yl]pyridine-3-carboxamide (100 mg, 159.25 μmol, 1 eq, HCl) in DCM (2 mL) was added TEA (80.57 mg, 796.24 μmol, 110.83 μL, 5 eq) and BrCN (230 mg, 2.17 mmol, 159.39 μL, 13.64 eq) at 0° C. The mixture was stirred at 0° C. for 0.25 hr. The reaction mixture was diluted with ice water (10 mL). The aqueous layer was extracted with ethyl acetate (10 mL*3). The combined organic layers were dried with anhydrous Na₂SO₄, filtered and concentrated in vacuum to give a residue. The residue was purified by prep-HPLC (column: Phenomenex Luna C18 150*25 mm*10 um; mobile phase: [water (NH₄HCO₃) -ACN]; gradient: 32%-62% B over 10 min), followed by lyophilization. Compound 5-[3-(4-cyano-2-methyl-piperazine-1-carbonyl)-1-(3,5-dichlorophenyl)-7-methoxy-4,5-dihydrobenzo[g]indazol-8-yl]pyridine-3-carboxamide (10.78 mg, 16.79 μmol, 11% yield, 96% purity) was obtained as an off-white solid. LCMS (ESI): m/z [M+H] calcd for C₃₁H₂₈Cl₂N₇O₃: 616.16; found: 616.3. ¹H NMR (400 MHz, METHANOL-d4) δ=8.88 (d, J=2.0 Hz, 1H), 8.54 (d, J=2.0 Hz, 1H), 8.22 (t, J=2.0 Hz, 1H), 7.69-7.59 (m, 3H), 7.23 (s, 1H), 6.87-6.83 (m, 1H), 4.96-4.91 (m, 2H), 4.52 (br s, 1H), 3.91 (s, 3H), 3.71-3.36 (m, 3H), 3.28-3.22 (m, 1H), 3.14-3.07 (m, 2H), 2.93-2.86 (m, 2H), 1.48 (d, J=6.8 Hz, 3H). FIG. 70 shows the nuclear magnetic resonance of Compound 8-09.

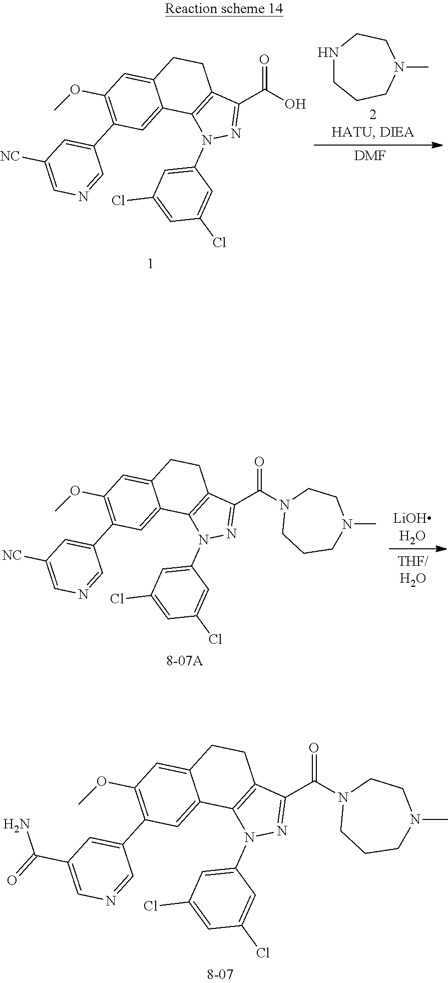

### Synthesis of 5-(1-(3,5-dichlorophenyl)-7-methoxy-3-(4-methyl-1,4-diazepane-1-carbonyl)-4,5-dihydro-1H-benzo[g]indazol-8-yl)nicotinonitrile

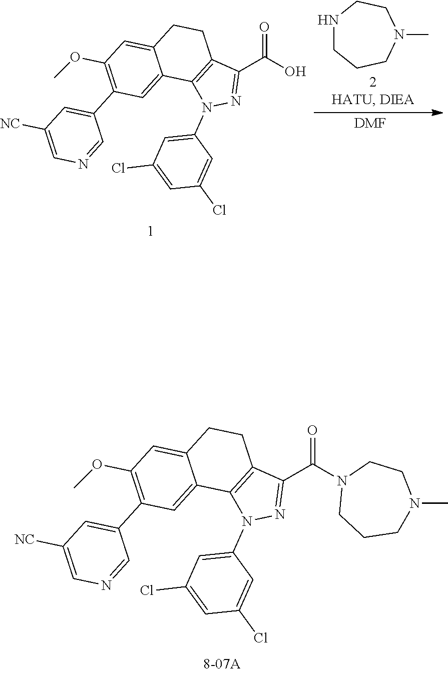

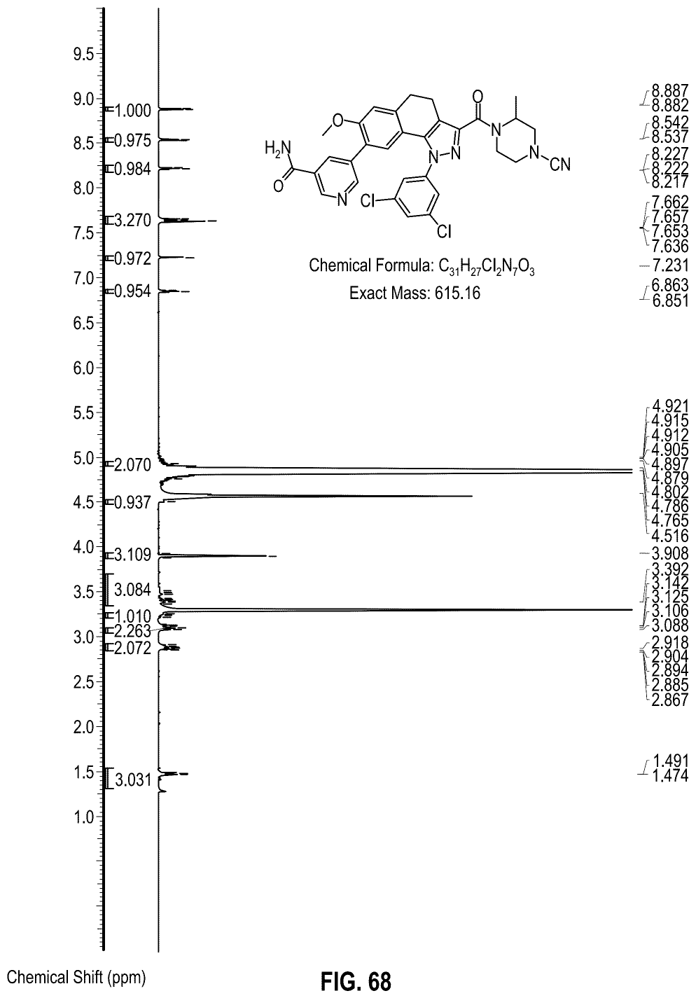

To a mixture of 8-(5-cyano-3-pyridyl)-1-(3,5-dichlorophenyl)-7-methoxy-4,5-dihydrobenzo[g]indazole-3-carboxylic acid (150 mg, 305.30 μmol, 1 eq) in DMF (3 mL) was added HATU (174.12 mg, 457.95 μmol, 1.5 eq) and DIEA (118.37 mg, 915.89 μmol, 159.53 μL, 3 eq). The mixture was stirred at 15° C. for 15 min, then 1-methyl-1,4-diazepane (62.75 mg, 549.53 μmol, 68.36 μL, 1.8 eq) was added. The mixture was stirred at 15° C. for 16 h. The mixture was poured into H₂O (10 mL). There was precipitate formed in the reaction mixture, the mixture was filtered and the filter cake was collected and dried in vacuum to a residue. The residue was purified by flash silica gel chromatography (ISCO®; 4 g SepaFlash® Silica Flash Column, Eluent of 0˜10% Ethyl acetate: Methanol @18 mL/min) to afford 5-(1-(3,5-dichlorophenyl)-7-methoxy-3-(4-methyl-1,4-diazepane-1-carbonyl)-4,5-dihydro-1H-benzo[g]indazol-8-yl)nicotinonitrile (90 mg, 153.19 mol, 50.18% yield) as a yellow solid. LCMS (ESI): m/z [M+H] calcd for C₃₃H₂₉N₆O₄Cl₂F₃: 587.17; found: 587.2. ¹H NMR (400 MHz, METHANOL-d4) δ=8.80 (d, J=1.8 Hz, 1H), 8.68 (s, 1H), 8.08 (br s, 1H), 7.75-7.61 (m, 3H), 7.24 (s, 1H), 6.86 (d, J=13.9 Hz, 1H), 4.33-3.96 (m, 2H), 3.92 (s, 3H), 3.87-3.72 (m, 2H), 3.71-3.49 (m, 2H), 3.48-3.32 (m, 2H), 3.14-3.07 (m, 2H), 3.02-2.91 (m, 5H), 2.39-2.20 (m, 2H). FIG. 68 shows the nuclear magnetic resonance of Compound 8-07A.

### Synthesis of 5-[1-(3,5-dichlorophenyl)-7-methoxy-3-(4-methyl-1,4-diazepane-1-carbonyl)-4,5-dihydrobenzo[g]indazol-8-yl]pyridine-3-carboxamide

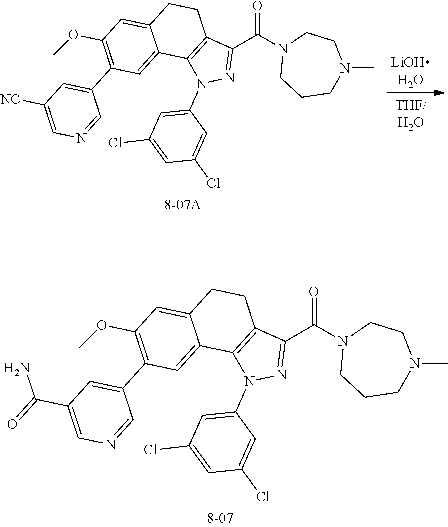 [SMILES: COc1cc2c(cc1-c1cncc(C#N)c1)-c1c(c(C(=O)N3CCCN(C)CC3)nn1-c1cc(Cl)cc(Cl)c1)CC2 ; COc1cc2c(cc1-c1cncc(C(N)=O)c1)-c1c(c(C(=O)N3CCCN(C)CC3)nn1-c1cc(Cl)cc(Cl)c1)CC2]

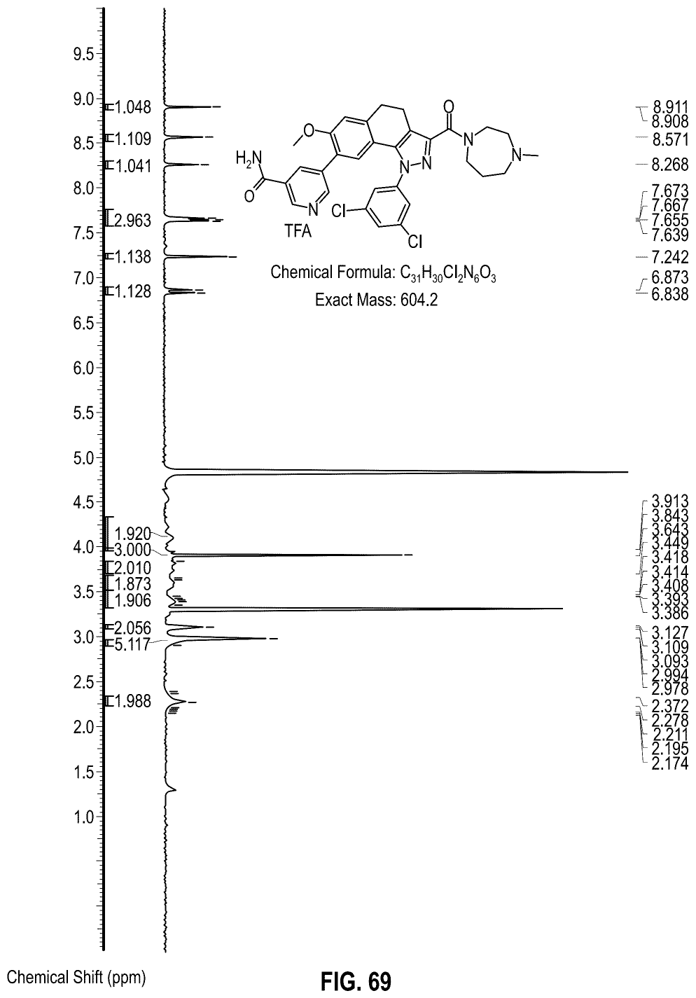

To a mixture of 5-(1-(3,5-dichlorophenyl)-7-methoxy-3-(4-methyl-1,4-diazepane-1-carbonyl)-4,5-dihydro-1H-benzo[g]indazol-8-yl)nicotinonitrile (90 mg, 153.19 μmol, 1 eq) in THF (1 mL) and H₂O (1 mL) was added LiOH·H₂O (6.43 mg, 153.19 μmol, 1 eq). The mixture was stirred at 60° C. for 16 h. The pH of the reaction mixture was adjusted to 5 with 1 M HCl aqueous solution. The resulting mixture was extracted with ethyl acetate (3 mL*3). The combined organic phase was dried over anhydrous Na₂SO₄ and filtered. The filtrate was concentrated under vacuum to give a residue. The residue was purified by prep-HPLC (column: Phenomenex Luna C18 150*25 mm*10 um;mobile phase: [water(TFA)-ACN];gradient:18%-48% B over 9 min) followed by lyophilization to afford 5-[1-(3,5-dichlorophenyl)-7-methoxy-3-(4-methyl-1,4-diazepane-1-carbonyl)-4,5-dihydrobenzo[g]indazol-8-yl]pyridine-3-carboxamide (24.6 mg, 34.19 μmol, 22% yield, 100% purity, TFA) as a white solid. LCMS (ESI): m/z [M+H] calcd for C₃₃H₃₁N₆O₅Cl₂F₃: 605.18; found: 605.2. ¹H NMR (400 MHz, METHANOL-d4) δ=8.91 (d, J=1.5 Hz, 1H), 8.57 (br s, 1H), 8.27 (br s, 1H), 7.70-7.61 (m, 3H), 7.24 (s, 1H), 6.86 (d, J=13.8 Hz, 1H), 4.10 (br s, 2H), 3.91 (s, 3H), 3.86-3.72 (m, 2H), 3.70-3.49 (m, 2H), 3.49-3.32 (m, 2H), 3.14-3.07 (m, 2H), 3.02-2.92 (m, 5H), 2.37-2.23 (m, 2H). FIG. 69 shows the nuclear magnetic resonance of Compound 8-07.

### Synthesis of Compound 8-02

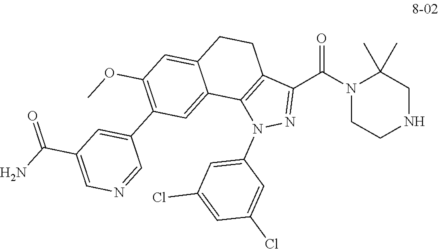 [SMILES: COc1cc2c(cc1-c1cncc(C(N)=O)c1)-c1c(c(C(=O)N3CCNCC3(C)C)nn1-c1cc(Cl)cc(Cl)c1)CC2]

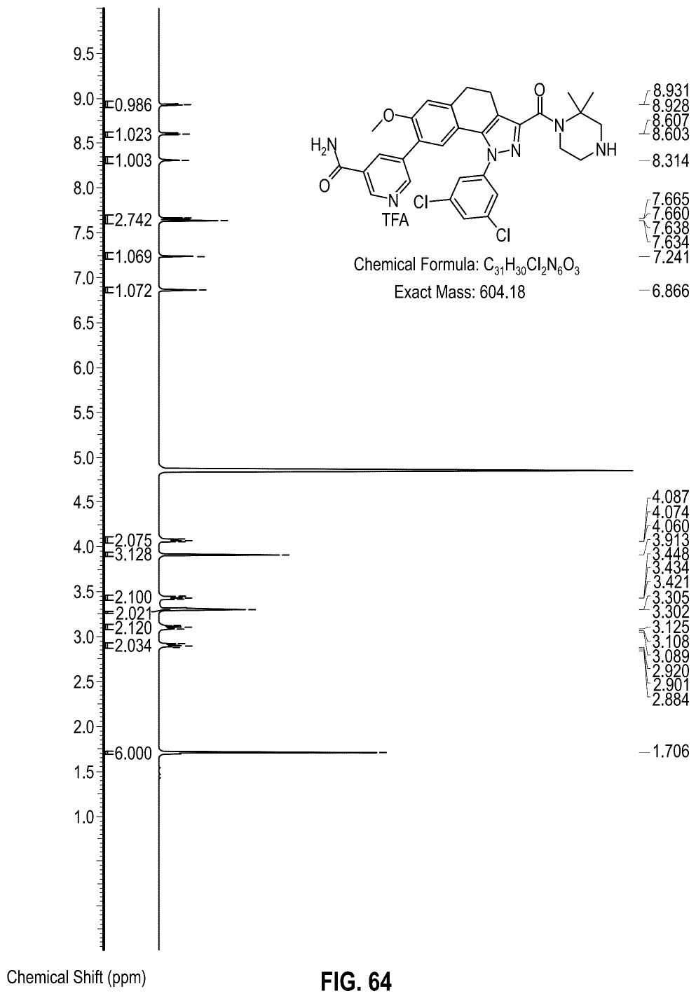

Compound 8-02 was synthesized via a similar procedure as example 8. LCMS (ESI): m/z [M+H] calcd for C₃₁H₃₁Cl₂N₆O₃: 605.18; found: 605.3. ¹H NMR (400 MHz, METHANOL-d4) δ=8.93 (d, J=1.6 Hz, 1H), 8.60 (d, J=1.6 Hz, 1H), 8.31 (s, 1H), 7.69-7.60 (m, 3H), 7.24 (s, 1H), 6.87 (s, 1H), 4.07 (br t, J=5.4 Hz, 2H), 3.91 (s, 3H), 3.43 (br t, J=5.4 Hz, 2H), 3.30 (br s, 2H), 3.14-3.07 (m, 2H), 2.93-2.87 (m, 2H), 1.71 (s, 6H). FIG. 64 shows the nuclear magnetic resonance of Compound 8-02.

### Synthesis of Compound 8-06

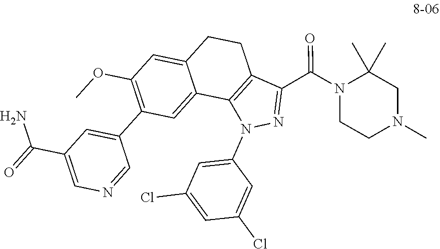 [SMILES: COc1cc2c(cc1-c1cncc(C(N)=O)c1)-c1c(c(C(=O)N3CCN(C)CC3(C)C)nn1-c1cc(Cl)cc(Cl)c1)CC2]

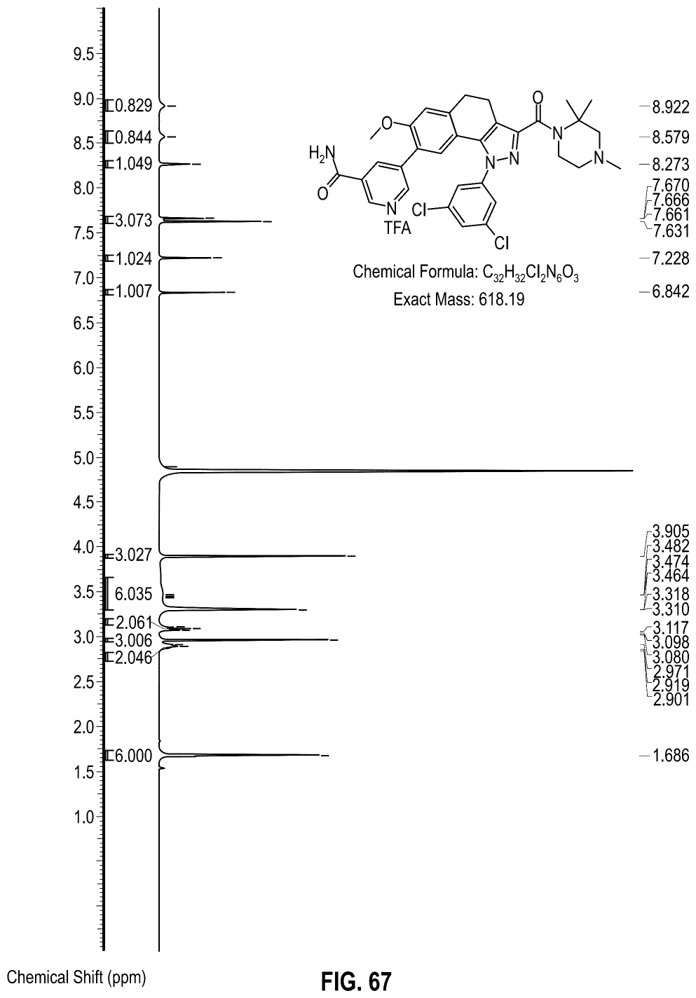

Compound 8-06 was synthesized via a similar procedure as example 8. LCMS (ESI): m/z [M+H] calcd for C₃₂H₃₃Cl₂N₆O₃ 619.19; found: 619.3. ¹H NMR (400 MHz, METHANOL-d4) δ=8.92 (br s, 1H), 8.58 (br s, 1H), 8.27 (s, 1H), 7.69-7.61 (m, 3H), 7.23 (s, 1H), 6.84 (s, 1H), 3.90 (s, 3H), 3.67-3.31 (m, 6H), 3.13-3.06 (m, 2H), 2.97 (s, 3H), 2.91 (br d, J=7.2 Hz, 2H), 1.69 (s, 6H). FIG. 67 shows the nuclear magnetic resonance of Compound 8-06.

### Synthesis of Compound 8-10

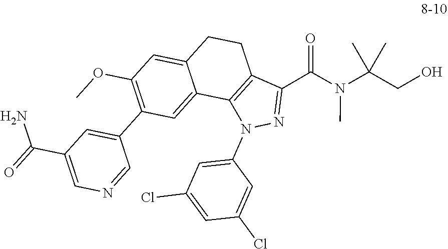 [SMILES: COc1cc2c(cc1-c1cncc(C(N)=O)c1)-c1c(c(C(=O)N(C)C(C)(C)CO)nn1-c1cc(Cl)cc(Cl)c1)CC2]

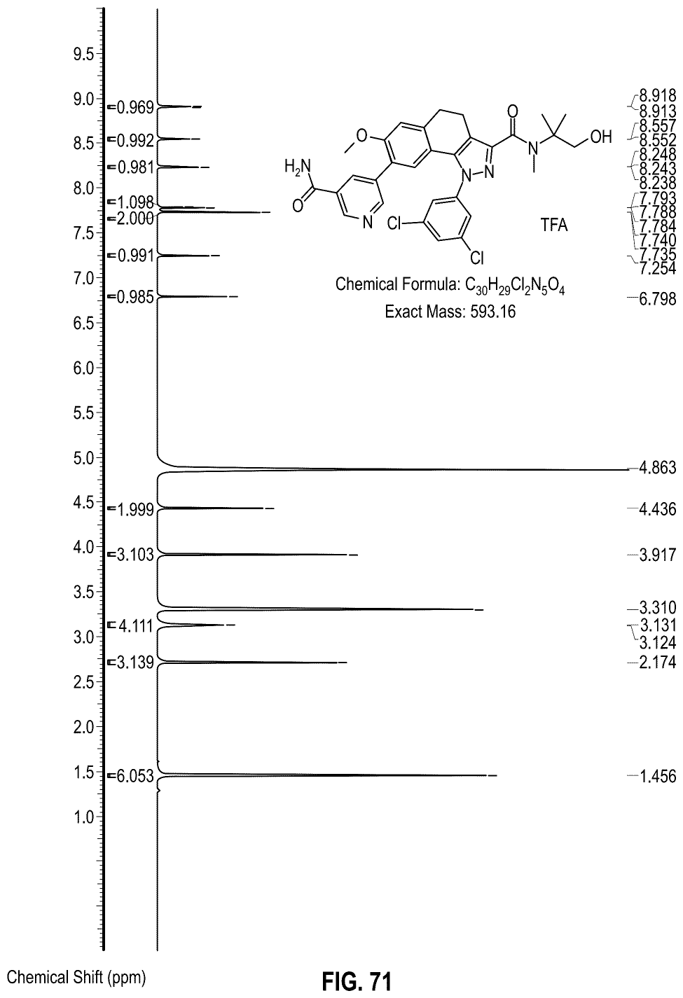

Compound 8-10 was synthesized via a similar procedure as example 8. LCMS (ESI): m/z [M+H] calcd for C₃₀H₃₀Cl₂N₅O₄: 594.16; found: 594.2. 1H NMR (400 MHz, METHANOL-d4) δ=8.92 (d, J=2.0 Hz, 1H), 8.55 (d, J=2.0 Hz, 1H), 8.24 (t, J=2.0 Hz, 1H), 7.79 (t, J=1.8 Hz, 1H), 7.74 (d, J=1.9 Hz, 2H), 7.25 (s, 1H), 6.80 (s, 1H), 4.44 (s, 2H), 3.92 (s, 3H), 3.17-3.10 (m, 4H), 2.71 (s, 3H), 1.46 (s, 6H). FIG. 71 shows the nuclear magnetic resonance of Compound 8-10.

### Synthesis of Compound 8-14

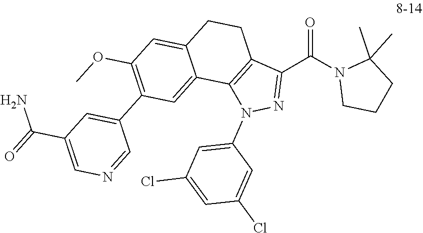 [SMILES: COc1cc2c(cc1-c1cncc(C(N)=O)c1)-c1c(c(C(=O)N3CCCC3(C)C)nn1-c1cc(Cl)cc(Cl)c1)CC2]

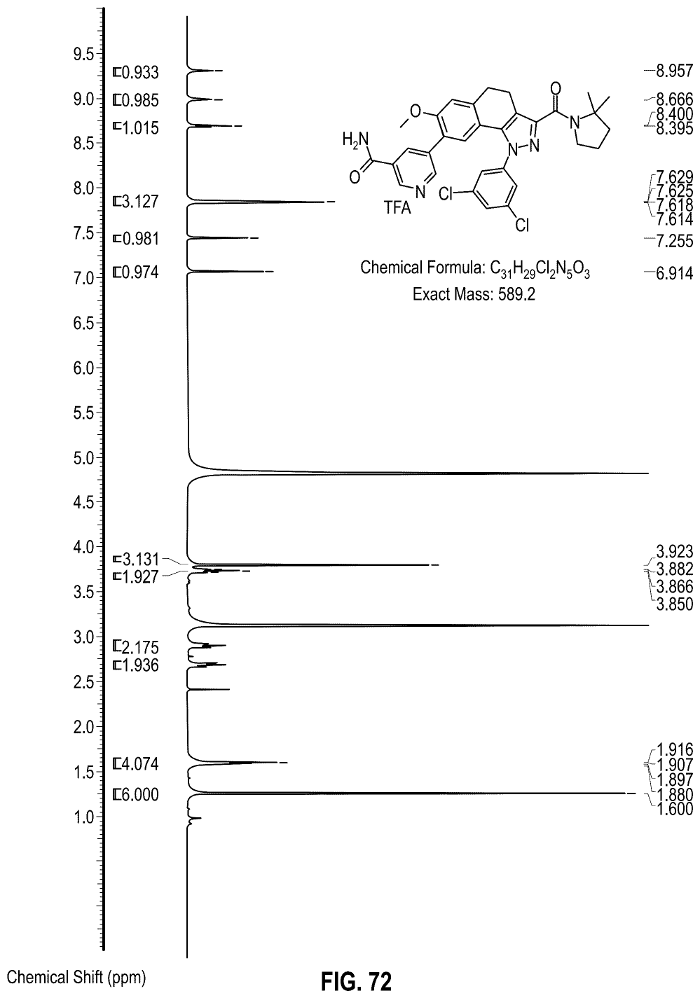

Compound 8-14 was synthesized via a similar procedure as example 8. LCMS (ESI): m/z [M+H] calcd for C₃₃H₃₀N₅O₅Cl₂F₃: 590.16; found: 590.2. ¹H NMR (400 MHz, METHANOL-d₄) δ=8.96 (br s, 1H), 8.67 (br s, 1H), 8.42-8.38 (m, 1H), 7.65-7.59 (m, 3H), 7.25 (s, 1H), 6.91 (s, 1H), 3.92 (s, 3H), 3.90-3.83 (m, 2H), 3.10 (br t, J=7.2 Hz, 2H), 2.91 (t, J=7.3 Hz, 2H), 1.96-1.85 (m, 4H), 1.60 (s, 6H). FIG. 72 shows the nuclear magnetic resonance of Compound 8-14.

### Synthesis of Compound 8-15

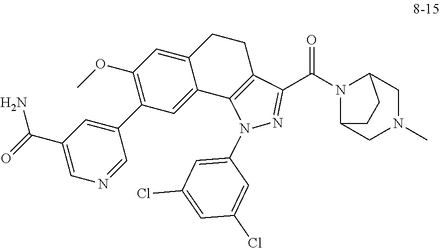 [SMILES: COc1cc2c(cc1-c1cncc(C(N)=O)c1)-c1c(c(C(=O)N3C4CCC3CN(C)C4)nn1-c1cc(Cl)cc(Cl)c1)CC2]

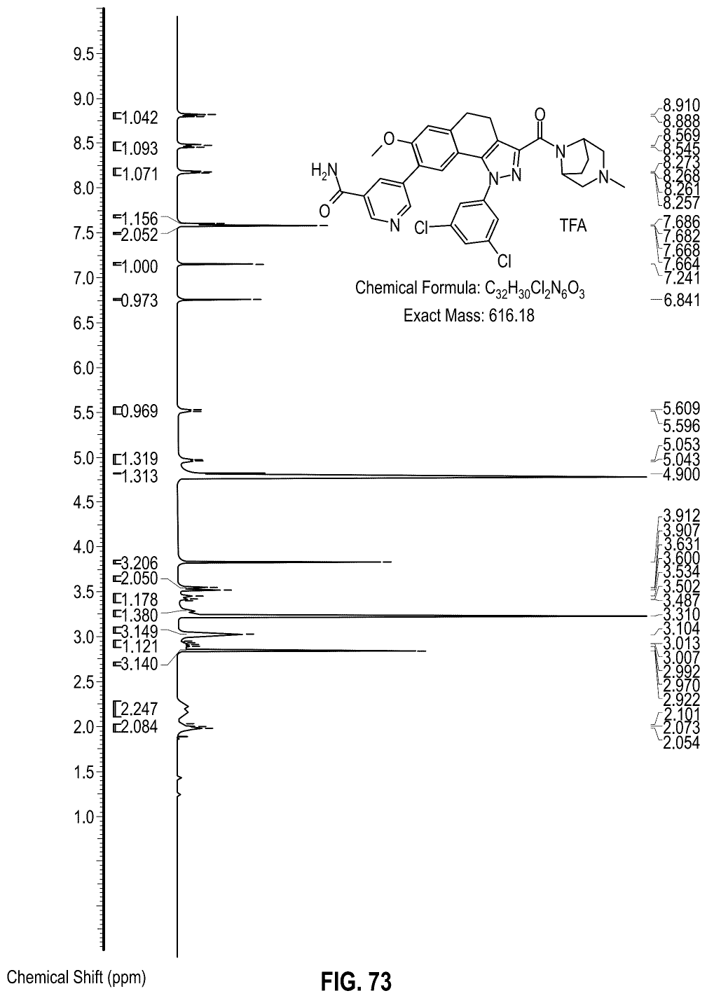

Compound 8-15 was synthesized via a similar procedure as example 8. LCMS (ESI): m/z [M+H] calcd for C₃₂H₃₁Cl₂N₆O₃: 617.18; found: 617.3. 1H NMR (400 MHz, METHANOL-d4) δ=8.93-8.86 (m, 1H), 8.56 (br d, J=9.3 Hz, 1H), 8.26 (dd, J=1.9, 4.8 Hz, 1H), 7.70-7.68 (m, 1H), 7.67 (d, J=1.6 Hz, 2H), 7.24 (s, 1H), 6.84 (d, J=1.5 Hz, 1H), 5.60 (br d, J=5.4 Hz, 1H), 5.05 (br d, J=4.1 Hz, 1H), 4.90 (s, 1H), 3.93-3.90 (m, 3H), 3.62 (br d, J=12.4 Hz, 2H), 3.56-3.46 (m, 1H), 3.40-3.34 (m, 1H), 3.10 (br s, 3H), 3.03-2.96 (m, 1H), 2.92 (s, 3H), 2.36-2.18 (m, 2H), 2.11-2.02 (m, 2H). FIG. 73 shows the nuclear magnetic resonance of Compound 8-15.

### Synthesis of Compound 8-17

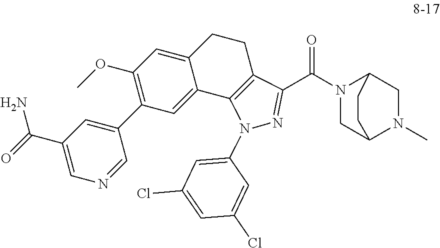 [SMILES: COc1cc2c(cc1-c1cncc(C(N)=O)c1)-c1c(c(C(=O)N3CC4CCC3CN4C)nn1-c1cc(Cl)cc(Cl)c1)CC2]

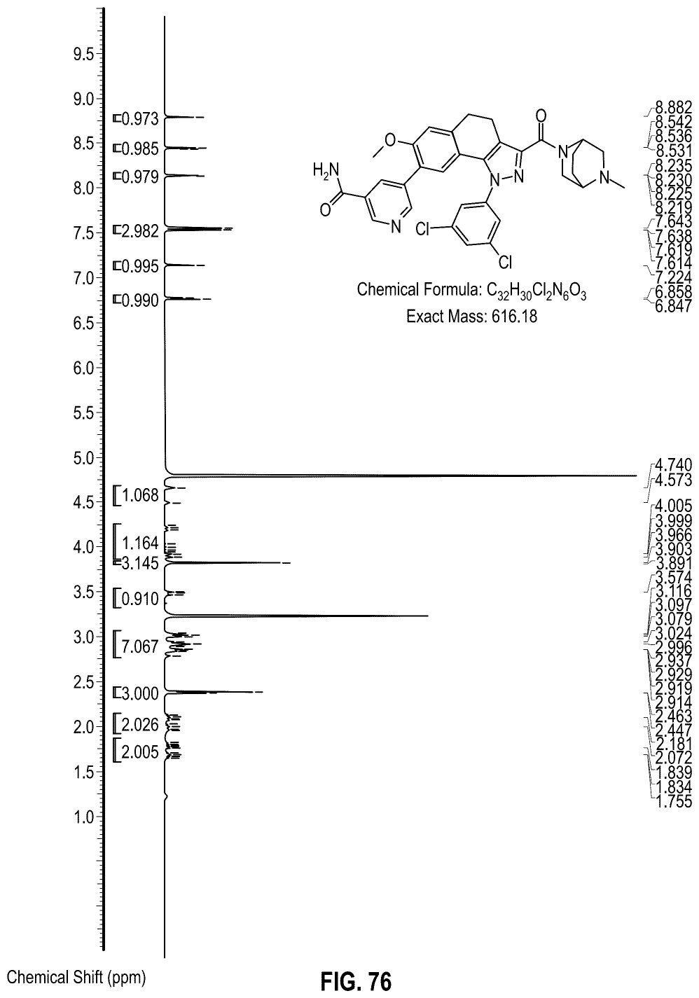

Compound 8-17 was synthesized via a similar procedure as example 8. LCMS (ESI): m/z [M+H] calcd for C₃₂H₃₁Cl₂N₆O₃: 617.18; found: 617.2. ¹H NMR (400 MHz, METHANOL-d4) δ=8.91-8.83 (m, 1H), 8.57-8.50 (m, 1H), 8.23 (q, J=2.2 Hz, 1H), 7.67-7.58 (m, 3H), 7.22 (s, 1H), 6.89-6.81 (m, 1H), 4.77-4.54 (m, 1H), 4.33-3.95 (m, 1H), 3.90 (s, 3H), 3.56 (dd, J=1.8, 13.2 Hz, 1H), 3.15-2.84 (m, 7H), 2.51-2.40 (m, 3H), 2.23-2.00 (m, 2H), 1.94-1.68 (m, 2H). FIG. 76 shows the nuclear magnetic resonance of Compound 8-17.

### Synthesis of Compound 8-20

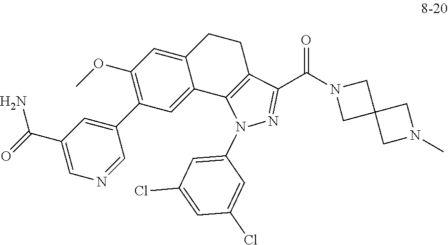 [SMILES: COc1cc2c(cc1-c1cncc(C(N)=O)c1)-c1c(c(C(=O)N3CC4(CN(C)C4)C3)nn1-c1cc(Cl)cc(Cl)c1)CC2]

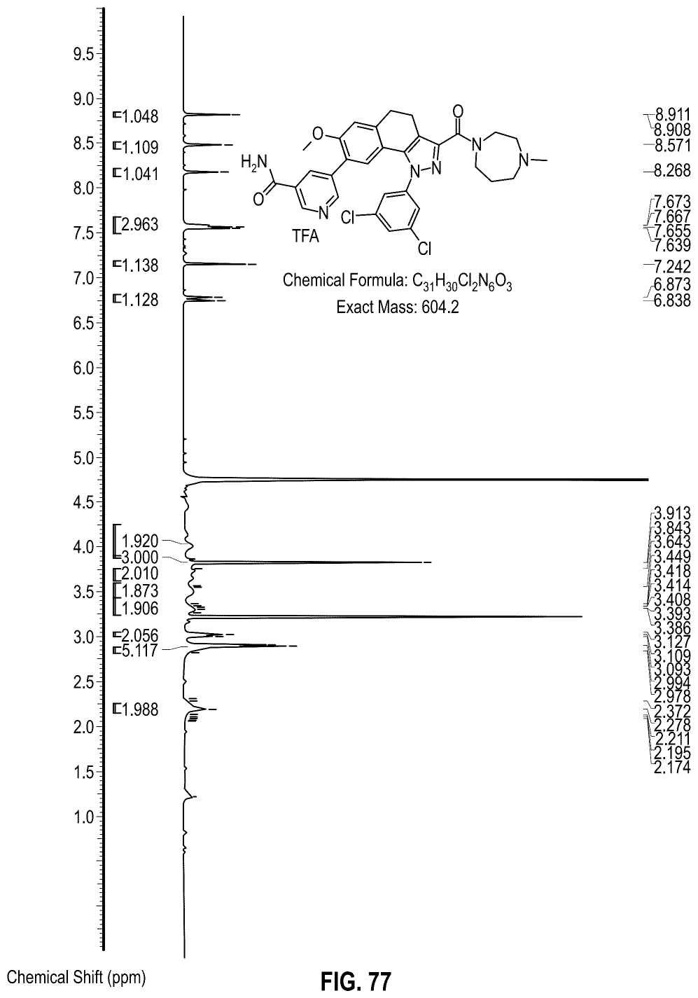

Compound 8-20 was synthesized via a similar procedure as example 8. LCMS (ESI): m/z [M+H] calcd for C31H29Cl2N6O3:603.16; found: 603.1. 1H NMR (400 MHz, METHANOL-d4) δ=8.94 (d, J=1.8 Hz, 1H), 8.61 (s, 1H), 8.33-8.28 (m, 1H), 7.70 (d, J=1.0 Hz, 1H), 7.68 (s, 2H), 7.26 (s, 1H), 6.91 (s, 1H), 4.93 (s, 2H), 4.57 (br s, 1H), 4.55 (s, 1H), 4.44 (s, 1H), 4.38 (s, 1H), 4.33-4.24 (m, 2H), 3.94 (s, 3H), 3.14-3.06 (m, 4H), 2.95 (br d, J=6.5 Hz, 3H). FIG. 77 shows the nuclear magnetic resonance of Compound 8-20.

### Synthesis of Compound 8-21

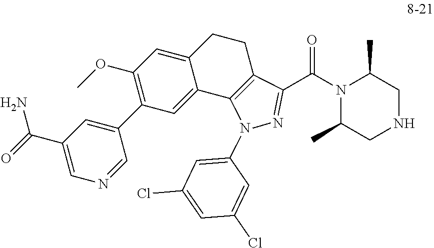 [SMILES: COc1cc2c(cc1-c1cncc(C(N)=O)c1)-c1c(c(C(=O)N3[C@H](C)CNC[C@@H]3C)nn1-c1cc(Cl)cc(Cl)c1)CC2]

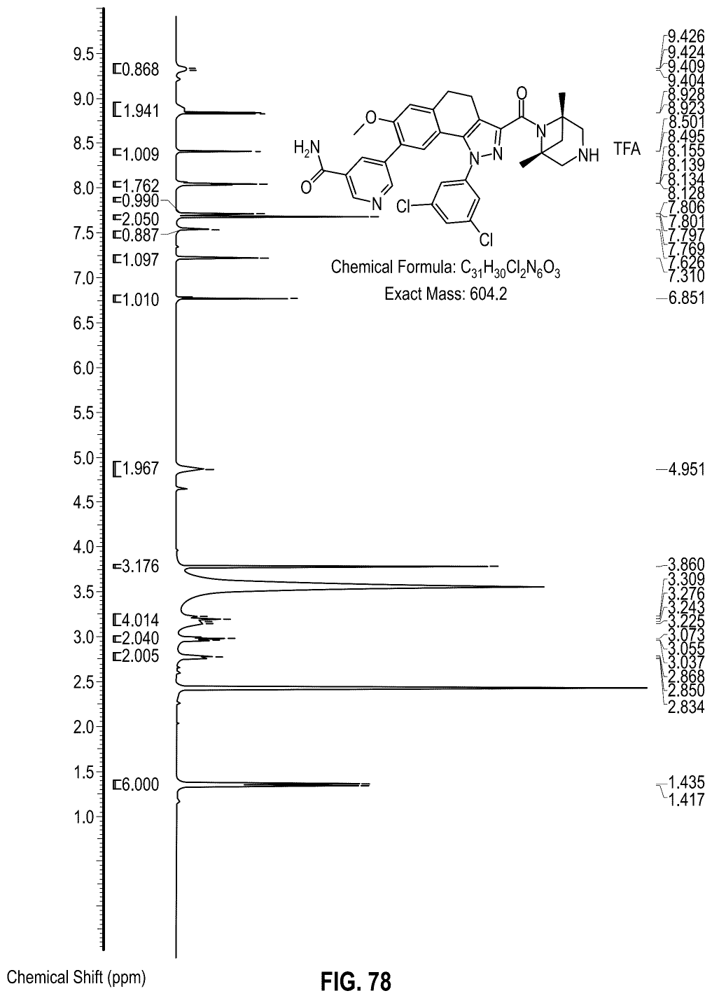

Compound 8-21 was synthesized via a similar procedure as example 8. LCMS (ESI): m/z [M+H] calcd for C₃₃H₃₁N₆O₅Cl₂F₃: 605.18; found: 605.2. ¹H NMR (400 MHz, DMSO-d₆) δ=9.42 (br dd, J=1.6, 7.4 Hz, 1H), 8.93 (d, J=2.0 Hz, 2H), 8.50 (d, J=2.1 Hz, 1H), 8.16-8.10 (m, 2H), 7.84-7.79 (m, 1H), 7.77 (d, J=1.8 Hz, 2H), 7.63 (s, 1H), 7.31 (s, 1H), 6.85 (s, 1H), 4.95 (br s, 2H), 3.86 (s, 3H), 3.33-3.20 (m, 4H), 3.09-3.02 (m, 2H), 2.85 (br t, J=6.9 Hz, 2H), 1.43 (d, J=7.1 Hz, 6H). FIG. 78 shows the nuclear magnetic resonance of Compound 8-21.

### Synthesis of Compound 8-22

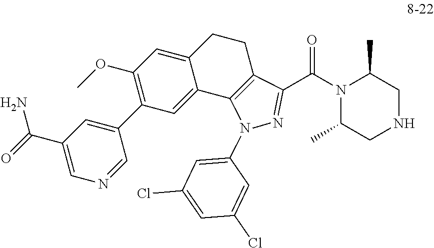 [SMILES: COc1cc2c(cc1-c1cncc(C(N)=O)c1)-c1c(c(C(=O)N3[C@@H](C)CNC[C@@H]3C)nn1-c1cc(Cl)cc(Cl)c1)CC2]

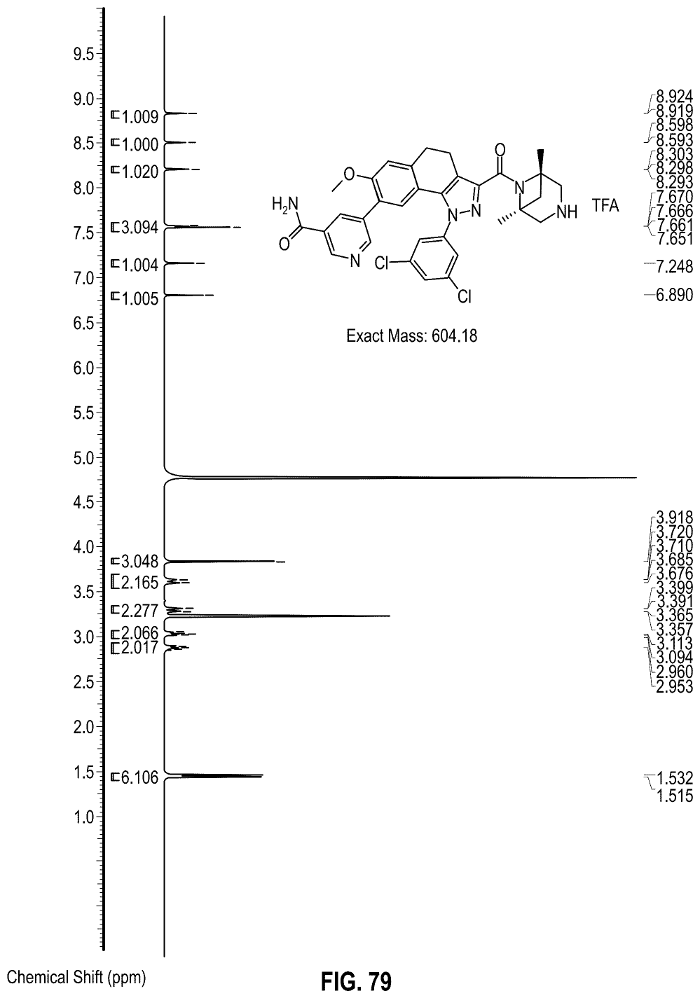

Compound 8-22 was synthesized via a similar procedure as example 8. LCMS (ESI): m/z [M+H] calcd for C31H31Cl2N6O3:605.18; found: 605.2. 1H NMR (400 MHz, METHANOL-d4) δ=8.92 (d, J=2.0 Hz, 1H), 8.60 (d, J=2.0 Hz, 1H), 8.30 (t, J=2.0 Hz, 1H), 7.70-7.60 (m, 3H), 7.25 (s, 1H), 6.89 (s, 1H), 3.92 (s, 3H), 3.70 (dd, J=3.9, 13.6 Hz, 2H), 3.38 (dd, J=3.2, 13.6 Hz, 2H), 3.15-3.06 (m, 2H), 3.01-2.89 (m, 2H), 1.52 (d, J=6.9 Hz, 6H). FIG. 79 shows the nuclear magnetic resonance of Compound 8-22.

### Synthesis of Compound 8-24

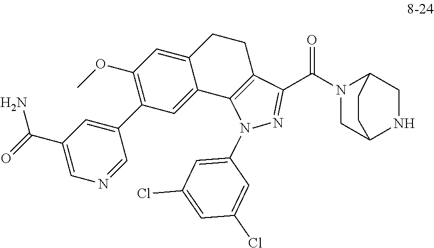 [SMILES: COc1cc2c(cc1-c1cncc(C(N)=O)c1)-c1c(c(C(=O)N3CC4CCC3CN4)nn1-c1cc(Cl)cc(Cl)c1)CC2]

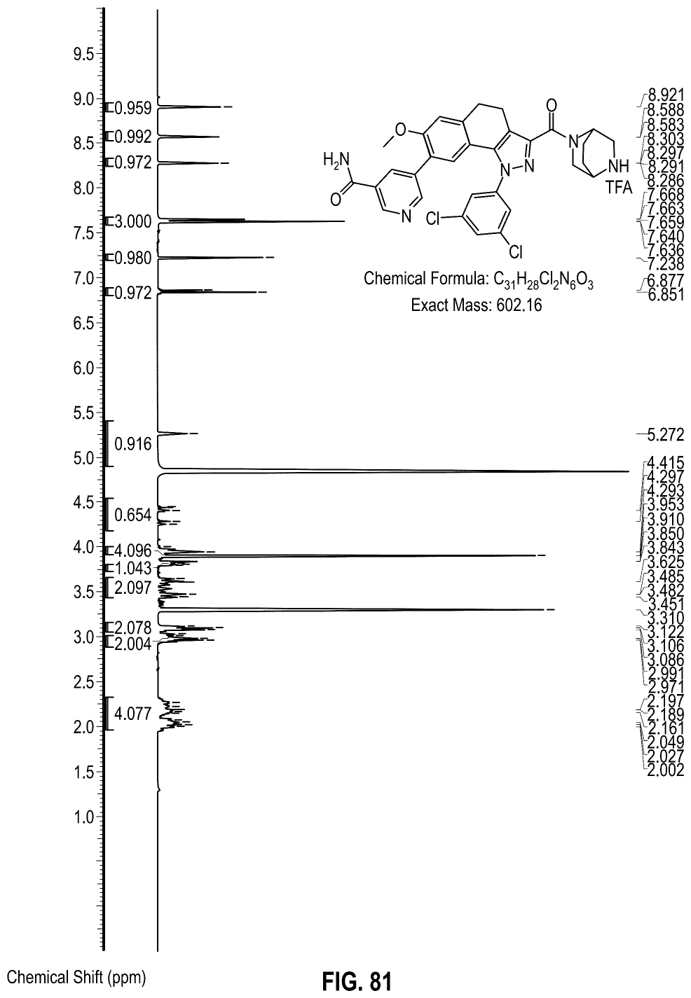

Compound 8-24 was synthesized via a similar procedure as example 8. LCMS (ESI): m/z [M+H] calcd for C₃₁H₂₉Cl₂N₆O₃: 603.16; found: 603.3. ¹H NMR (400 MHz, METHANOL-d4) δ=8.92 (s, 1H), 8.59 (d, J=1.9 Hz, 1H), 8.35-8.26 (m, 1H), 7.69-7.60 (m, 3H), 7.24 (s, 1H), 6.90-6.82 (m, 1H), 5.27 (br s, 1H), 4.55-4.19 (m, 1H), 3.91 (s, 4H), 3.87-3.78 (m, 1H), 3.67-3.44 (m, 2H), 3.17-3.06 (m, 2H), 3.05-2.92 (m, 2H), 2.33-1.97 (m, 4H). FIG. 81 shows the nuclear magnetic resonance of Compound 8-24.

### Synthesis of Compound 8-26A

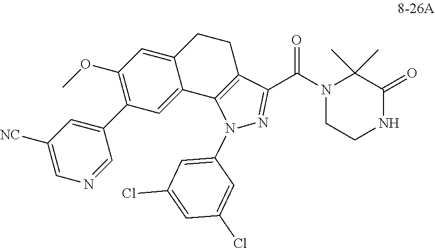 [SMILES: COc1cc2c(cc1-c1cncc(C#N)c1)-c1c(c(C(=O)N3CCNC(=O)C3(C)C)nn1-c1cc(Cl)cc(Cl)c1)CC2]

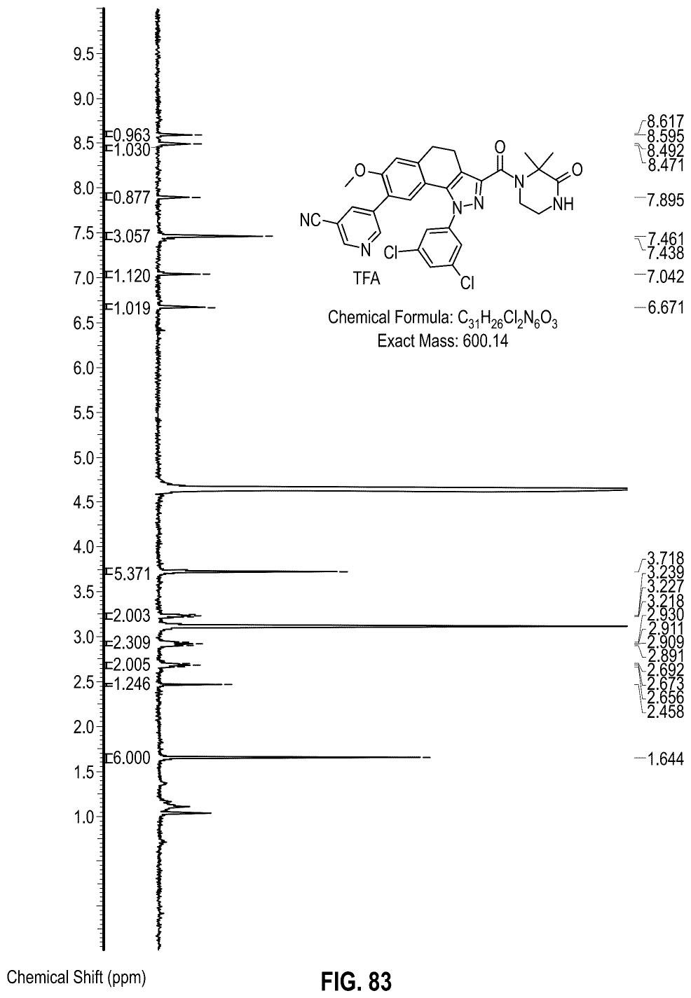

Compound 8-26A was synthesized via a similar procedure as example 8. LCMS (ESI): m/z [M+H] calcd for C31H27Cl2N6O3:601.14; found: 601.1. 1H NMR (400 MHz, METHANOL-d4) δ=8.60 (s, 1H), 8.52-8.46 (m, 1H), 7.90 (s, 1H), 7.46 (s, 3H), 7.04 (s, 1H), 6.67 (s, 1H), 3.72 (s, 5H), 3.26-3.19 (m, 2H), 2.94-2.89 (m, 2H), 2.67 (br t, J=7.2 Hz, 2H), 2.46 (s, 1H), 1.64 (s, 6H). FIG. 83 shows the nuclear magnetic resonance of Compound 8-26A.

### Synthesis of Compound 8-26

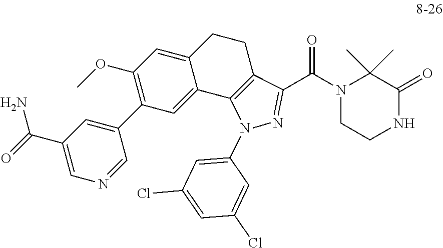 [SMILES: COc1cc2c(cc1-c1cncc(C(N)=O)c1)-c1c(c(C(=O)N3CCNC(=O)C3(C)C)nn1-c1cc(Cl)cc(Cl)c1)CC2]

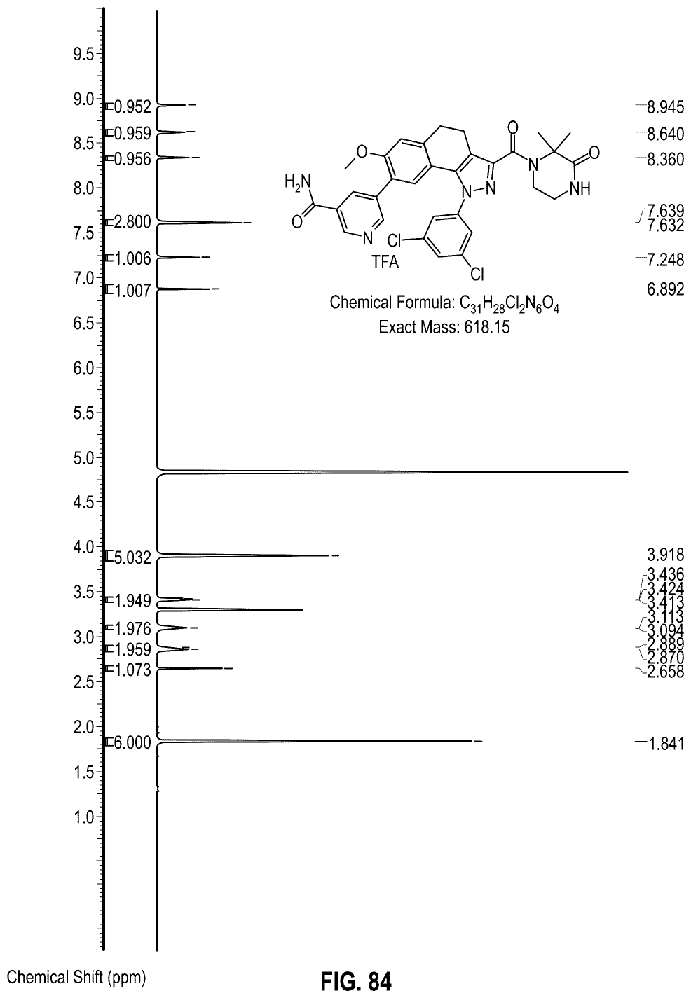

Compound 8-26 was synthesized via a similar procedure as example 8. LCMS (ESI): m/z [M+H] calcd for C31H29Cl2N6O4:619.15; found: 619.2. 1H NMR (400 MHz, METHANOL-d4) δ=8.60 (s, 1H), 8.52-8.46 (m, 1H), 7.90 (s, 1H), 7.46 (s, 3H), 7.04 (s, 1H), 6.67 (s, 1H), 3.72 (s, 5H), 3.26-3.19 (m, 2H), 2.94-2.89 (m, 2H), 2.67 (br t, J=7.2 Hz, 2H), 2.46 (s, 1H), 1.64 (s, 6H). FIG. 84 shows the nuclear magnetic resonance of Compound 8-26.

### Synthesis of Compound 8-28

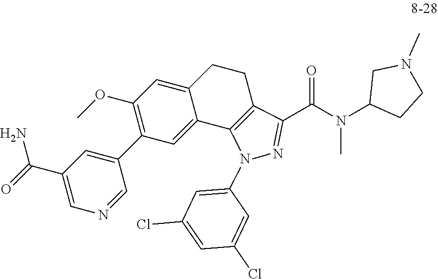 [SMILES: COc1cc2c(cc1-c1cncc(C(N)=O)c1)-c1c(c(C(=O)N(C)C3CCN(C)C3)nn1-c1cc(Cl)cc(Cl)c1)CC2]

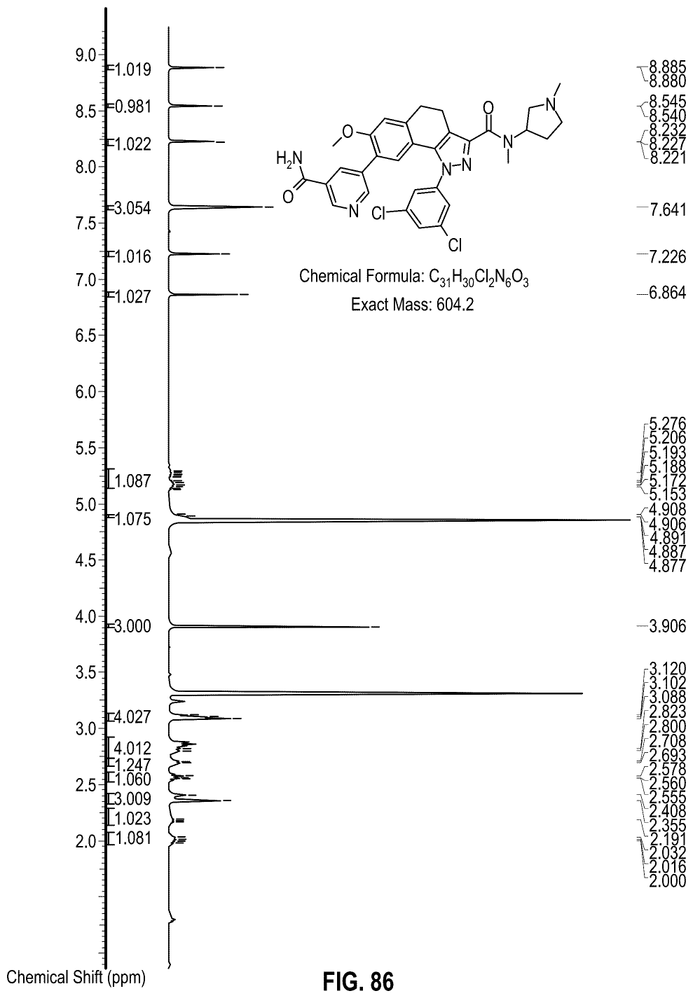

Compound 8-28 was synthesized via a similar procedure as example 8. LCMS (ESI): m/z [M+H] calcd for C₃₁H₃₀N₆O₃Cl₂: 605.18; found: 605.2. ¹H NMR (400 MHz, METHANOL-d₄) δ=8.88 (d, J=2.0 Hz, 1H), 8.54 (d, J=2.1 Hz, 1H), 8.23 (t, J=2.1 Hz, 1H), 7.64 (s, 3H), 7.23 (s, 1H), 6.86 (s, 1H), 5.31-5.13 (m, 1H), 4.90-4.87 (m, 1H), 3.91 (s, 3H), 3.13-3.07 (m, 4H), 2.81 (br d, J=9.0 Hz, 4H), 2.70 (br d, J=5.9 Hz, 1H), 2.61-2.53 (m, 1H), 2.42-2.33 (m, 3H), 2.29-2.14 (m, 1H), 2.07-1.97 (m, 1H). FIG. 86 shows the nuclear magnetic resonance of Compound 8-28.

### Synthesis of Compound 8-29

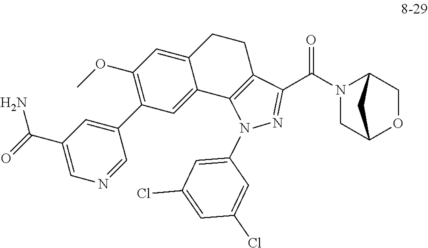 [SMILES: COc1cc2c(cc1-c1cncc(C(N)=O)c1)-c1c(c(C(=O)N3C[C@@H]4C[C@H]3CO4)nn1-c1cc(Cl)cc(Cl)c1)CC2]

Compound 8-29 was synthesized via a similar procedure as example 8. LCMS (ESI): m/z [M+H] calcd for C₃₂H₂₆N₅O₆Cl₂F₃: 590.13; found: 590.2. 1H NMR (400 MHz, METHANOL-d4) δ=8.94 (d, J=1.9 Hz, 1H), 8.63 (s, 1H), 8.35 (q, J=2.0 Hz, 1H), 7.73-7.61 (m, 3H), 7.25 (s, 1H), 6.89 (d, J=4.6 Hz, 1H), 5.60 (s, 0.5H), 5.06 (s, 0.5H), 4.70 (br d, J=12.0 Hz, 1H), 4.01-3.86 (m, 6H), 3.64-3.52 (m, 1H), 3.16-3.08 (m, 2H), 3.08-2.91 (m, 2H), 1.98 (br d, J=4.1 Hz, 2H). FIG. 81 shows the nuclear magnetic resonance of Compound 8-24.

### Synthesis of Compound 8-30

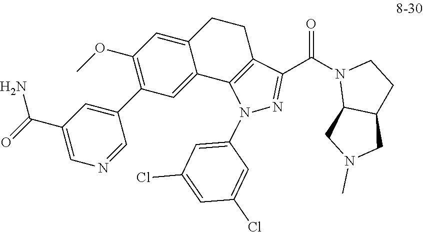 [SMILES: COc1cc2c(cc1-c1cncc(C(N)=O)c1)-c1c(c(C(=O)N3CC[C@H]4CN(C)C[C@H]43)nn1-c1cc(Cl)cc(Cl)c1)CC2]

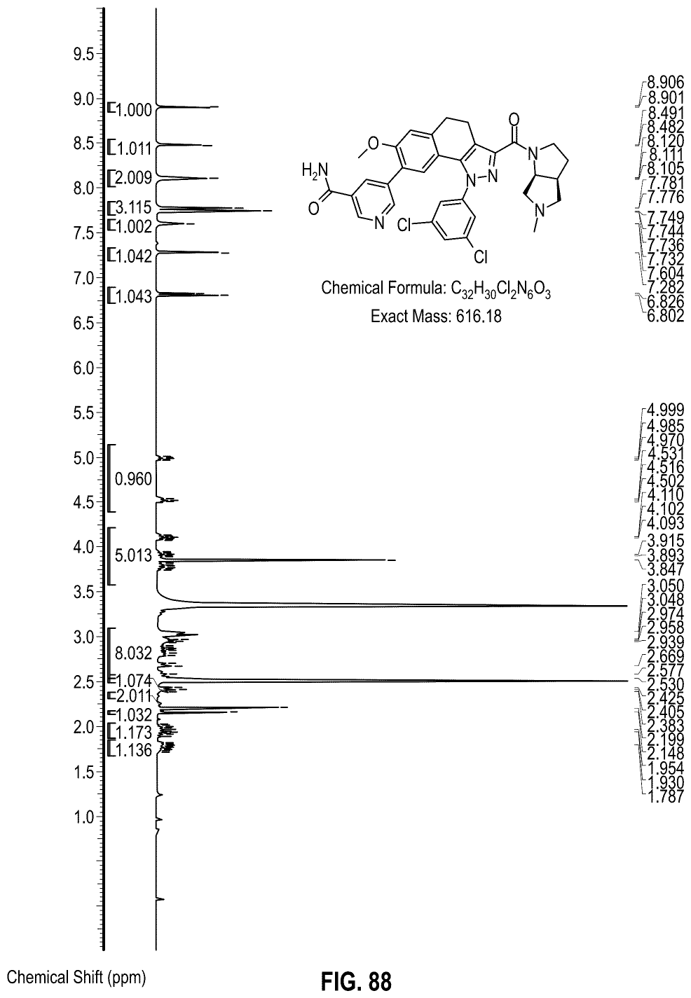

Compound 8-30 was synthesized via a similar procedure as example 8. LCMS (ESI): m/z [M+H] calcd for C₃₂H₃₁Cl₂N₆O₃:617.18; found: 617.1. 1H NMR (400 MHz, DMSO-d6) δ=8.90 (d, J=2.0 Hz, 1H), 8.54-8.38 (m, 1H), 8.20-8.01 (m, 2H), 7.85-7.70 (m, 3H), 7.60 (s, 1H), 7.28 (s, 1H), 6.90-6.72 (m, 1H), 5.13-4.39 (m, 1H), 4.21-3.58 (m, 5H), 3.09-2.52 (m, 8H), 2.44-2.36 (m, 1H), 2.20 (s, 2H), 2.15 (s, 1H), 2.03-1.86 (m, 1H), 1.84-1.67 (m, 1H). FIG. 88 shows the nuclear magnetic resonance of Compound 8-30.

### Synthesis of Compound 8-32

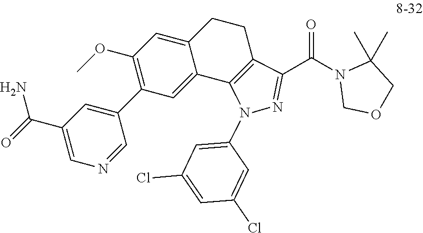 [SMILES: COc1cc2c(cc1-c1cncc(C(N)=O)c1)-c1c(c(C(=O)N3COCC3(C)C)nn1-c1cc(Cl)cc(Cl)c1)CC2]

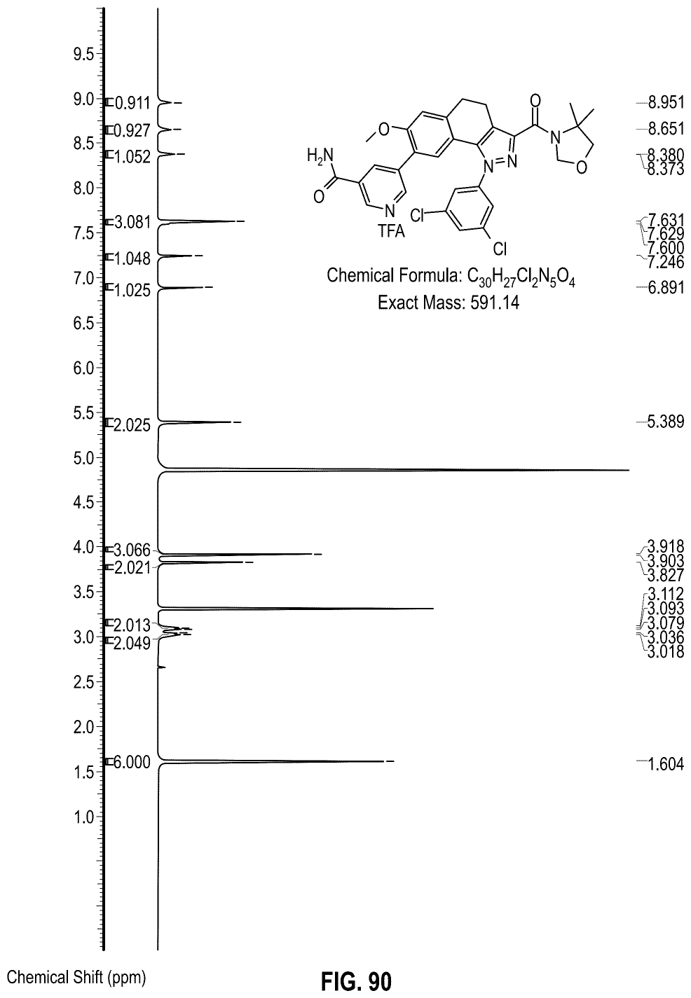

Compound 8-32 was synthesized via a similar procedure as example 8. LCMS (ESI): m/z [M+H] calcd for C₃₀H₂₈Cl₂N₅O₄: 592.14; found: 592.3. ¹H NMR (400 MHz, METHANOL-d4) δ=8.95 (br s, 1H), 8.65 (br s, 1H), 8.42-8.34 (m, 1H), 7.65-7.59 (m, 3H), 7.25 (s, 1H), 6.89 (s, 1H), 5.39 (s, 2H), 3.95-3.89 (m, 3H), 3.83 (s, 2H), 3.13-3.06 (m, 2H), 3.03 (br d, J=7.2 Hz, 2H), 1.60 (s, 6H). FIG. 90 shows the nuclear magnetic resonance of Compound 8-32.

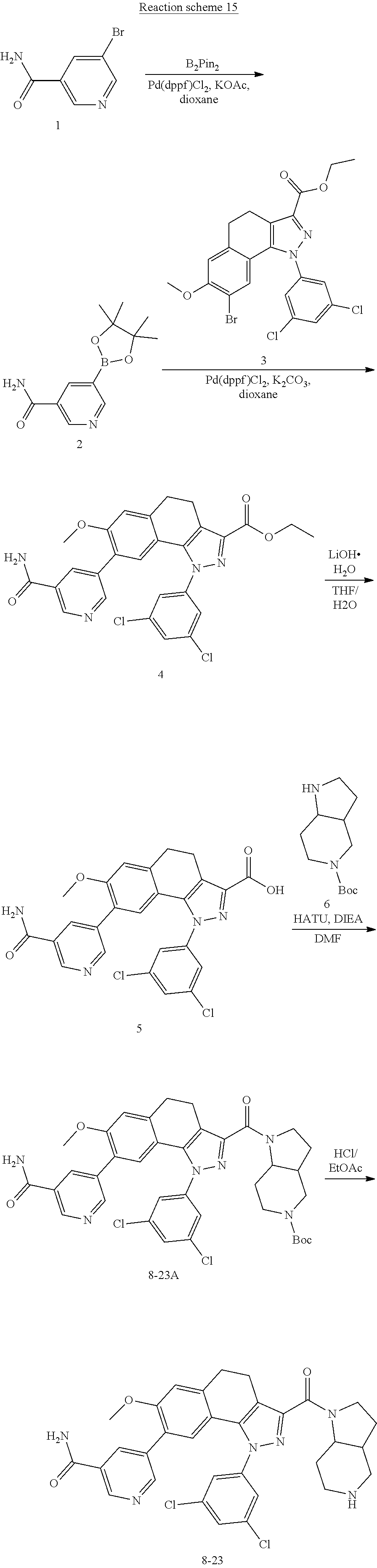

### Synthesis of 5-(4,4,5,5-tetramethyl-1,3,2-dioxaborolan-2-yl)nicotinamide

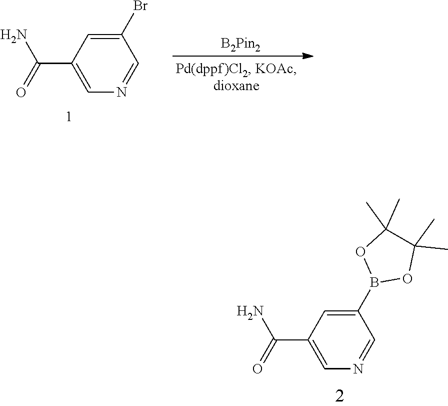 [SMILES: NC(=O)c1cncc(Br)c1 ; CC1(C)OB(c2cncc(C(N)=O)c2)OC1(C)C]

To a mixture of 5-bromopyridine-3-carboxamide (2 g, 9.95 mmol, 1 eq) in dioxane (20 mL) was added Pd(dppf)Cl₂ (727.99 mg, 994.92 μmol, 0.1 eq), KOAc (1.95 g, 19.90 mmol, 2 eq) and 4,4,5,5-tetramethyl-2-(4,4,5,5-tetramethyl-1,3,2-dioxaborolan-2-yl)-1,3,2-dioxaborolane (3.79 g, 14.92 mmol, 1.5 eq), the mixture was stirred at 100° C. for 3 h under N2 atmosphere. 5-(4,4,5,5-tetramethyl-1,3,2-dioxaborolan-2-yl)nicotinamide (1.65 g, crude) in dioxane as a brown liquid was used to next step directly.

### Synthesis of ethyl 8-(5-carbamoyl-3-pyridyl)-1-(3,5-dichlorophenyl)-7-methoxy-4,5-dihydrobenzo[g]indazole-3-carboxylate

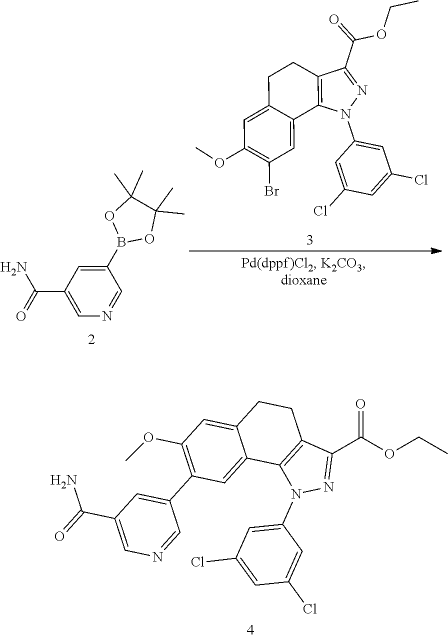 [SMILES: CC1(C)OB(c2cncc(C(N)=O)c2)OC1(C)C ; CCOC(=O)c1nn(-c2cc(Cl)cc(Cl)c2)c2c1CCc1cc(OC)c(Br)cc1-2 ; CCOC(=O)c1nn(-c2cc(Cl)cc(Cl)c2)c2c1CCc1cc(OC)c(-c3cncc(C(N)=O)c3)cc1-2]

To a mixture of 5-(4,4,5,5-tetramethyl-1,3,2-dioxaborolan-2-yl)nicotinamide (1.34 g, 5.4 mmol, 1.35 eq) and ethyl 8-bromo-1-(3,5-dichlorophenyl)-7-methoxy-4,5-dihydrobenzo[g]indazole-3-carboxylate (2 g, 4.03 mmol, 1 eq) in dioxane (10 mL) and H₂O (4 mL) was added K₂CO₃ (1.11 g, 8.06 mmol, 2 eq) and Pd(dppf)Cl₂ (589.87 mg, 806.16 μmol, 0.2 eq). The mixture was stirred at 60° C. for 16 h under N2 atmosphere. The reaction mixture was poured into H₂O (20 mL) and ethyl acetate (20 mL), then the mixture was separated. The aqueous phase was extracted with ethyl acetate (10 mL*3). The combined organic phase was dried over anhydrous Na₂SO₄ and filtered. The filtrate was concentrated under vacuum to give a residue. The residue was purified by flash silica gel chromatography (ISCO®; 40 g SepaFlash® Silica Flash Column, Eluent of 0˜80% Petroleum ethergradient/Ethylacetate @45 mL/min) to afford ethyl 8-(5-carbamoyl-3-pyridyl)-1-(3,5-dichlorophenyl)-7-methoxy-4,5-dihydrobenzo[g]indazole-3-carboxylate (1.2 g, 2.23 mmol, 55% yield) as a gray solid.

### Synthesis of 8-(5-carbamoyl-3-pyridyl)-1-(3,5-dichlorophenyl)-7-methoxy-4,5-dihydrobenzo[g]indazole-3-carboxylic acid

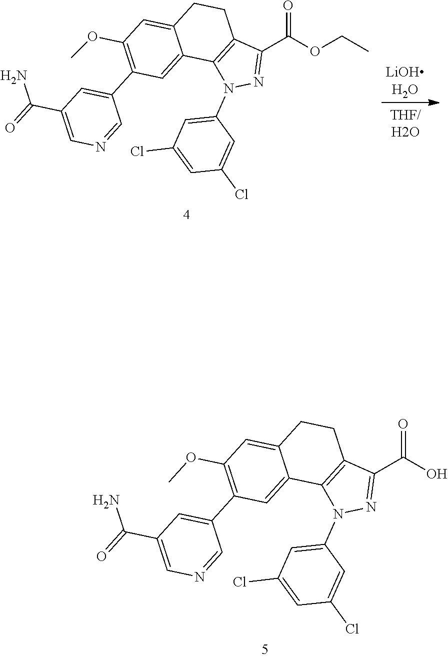 [SMILES: CCOC(=O)c1nn(-c2cc(Cl)cc(Cl)c2)c2c1CCc1cc(OC)c(-c3cncc(C(N)=O)c3)cc1-2 ; COc1cc2c(cc1-c1cncc(C(N)=O)c1)-c1c(c(C(=O)O)nn1-c1cc(Cl)cc(Cl)c1)CC2]

To a mixture of ethyl 8-(5-carbamoyl-3-pyridyl)-1-(3,5-dichlorophenyl)-7-methoxy-4,5-dihydrobenzo[g]indazole-3-carboxylate (1.2 g, 2.23 mmol, 1 eq) in THF (10 mL) and H₂O (10 mL) was added LiOH·H₂O (281.11 mg, 6.70 mmol, 3 eq).The mixture was stirred at 15° C. for 16 h. The mixture was concentrated under vacuum to remove THF, then the resulting mixture was filtered. The filtered cake was dried under vacuum to afford 8-(5-carbamoyl-3-pyridyl)-1-(3,5-dichlorophenyl)-7-methoxy-4,5-dihydrobenzo[g]indazole-3-carboxylic acid (1 g, 1.96 mmol, 88% yield) as a gray solid. 1H NMR (400 MHz, DMSO-d6) δ=8.90 (d, J=2.1 Hz, 1H), 8.47 (d, J=2.0 Hz, 1H), 8.14-8.02 (m, 2H), 7.79 (d, J=1.8 Hz, 1H), 7.73 (d, J=1.8 Hz, 2H), 7.60 (br s, 1H), 7.28 (s, 1H), 6.78 (s, 1H), 3.84 (s, 3H), 3.04-2.92 (m, 4H).

### Synthesis of tert-butyl 1-[8-(5-carbamoyl-3-pyridyl)-1-(3,5-dichlorophenyl)-7-methoxy-4,5-dihydrobenzo[g]indazole-3-carbonyl]-3,3a,4,6,7,7a-hexahydro-2H-pyrrolo[3,2-c]pyridine-5-carboxylate

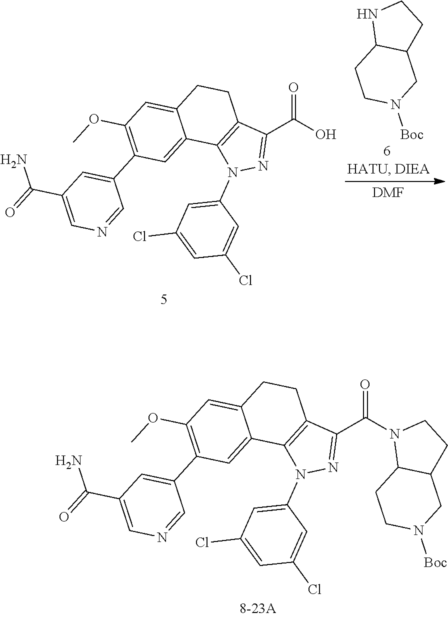

To a mixture of 8-(5-carbamoyl-3-pyridyl)-1-(3,5-dichlorophenyl)-7-methoxy-4,5-dihydrobenzo[g]indazole-3-carboxylic acid (80 mg, 157.07 μmol, 1 eq) in DMF (2 mL)was added HATU (89.58 mg, 235.60 μmol, 1.5 eq) and DIEA (60.90 mg, 471.20 μmol, 82.07 μL, 3 eq). The mixture was stirred at 15° C. for 15 min, then tert-butyl 1,2,3,3a,4,6,7,7a-octahydropyrrolo[3,2-c]pyridine-5-carboxylate (35.55 mg, 157.07 μmol, 1 eq) was added. The mixture was stirred at 15° C. for 16 h. The pH of the mixture was adjusted to 6 with TFA. The residue was purified by prep-HPLC (column: Phenomenex Luna C18 150*25 mm*10 um;mobile phase: [water (TFA) -ACN];gradient: 52%-82% B over 9 min) followed by lyophilization to afford tert-butyl 1-[8-(5-carbamoyl-3-pyridyl)-1-(3,5-dichlorophenyl)-7-methoxy-4,5-dihydrobenzo[g]indazole-3-carbonyl]-3,3a,4,6,7,7a-hexahydro-2H-pyrrolo[3,2-c]pyridine-5-carboxylate (20 mg, 23.33 μmol, 15% yield, 97% purity, TFA) as a white solid. 1H NMR (400 MHz, METHANOL-d4) δ=8.97 (d, J=1.6 Hz, 1H), 8.68 (s, 1H), 8.42 (s, 1H), 7.63 (d, J=5.8 Hz, 3H), 7.25 (s, 1H), 6.92 (d, J=6.9 Hz, 1H), 4.84-4.75 (m, 0.5H), 4.49-4.38 (m, 0.5H), 4.18-4.05 (m, 1H), 4.03-3.88 (m, 5H), 3.78-3.66 (m, 1H), 3.12-3.06 (m, 2H), 3.04-2.85 (m, 3H), 2.70 (s, 1H), 2.48-2.30 (m, 1H), 2.28-2.14 (m, 1H), 2.04-1.90 (m, 2H), 1.69-1.47 (m, 2H), 1.46 (d, J=1.6 Hz, 9H). LCMS (ESI): m/z [M+H] calcd for C₃₉H₃₉N₆O₇Cl₂F₃: 717.23; found: 717.2.

### Synthesis of 5-[3-(2,3,3a,4,5,6,7,7a-octahydropyrrolo[3,2-c]pyridine-1-carbonyl)-1-(3,5-dichlorophenyl)-7-methoxy-4,5-dihydrobenzo[g]indazol-8-yl]pyridine-3-carboxamide

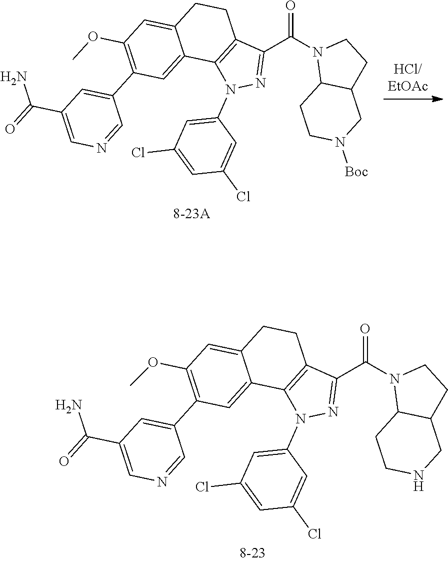 [SMILES: COc1cc2c(cc1-c1cncc(C(N)=O)c1)-c1c(c(C(=O)N3CCC4CN(C(=O)OC(C)(C)C)CCC43)nn1-c1cc(Cl)cc(Cl)c1)CC2 ; COc1cc2c(cc1-c1cncc(C(N)=O)c1)-c1c(c(C(=O)N3CCC4CNCCC43)nn1-c1cc(Cl)cc(Cl)c1)CC2]

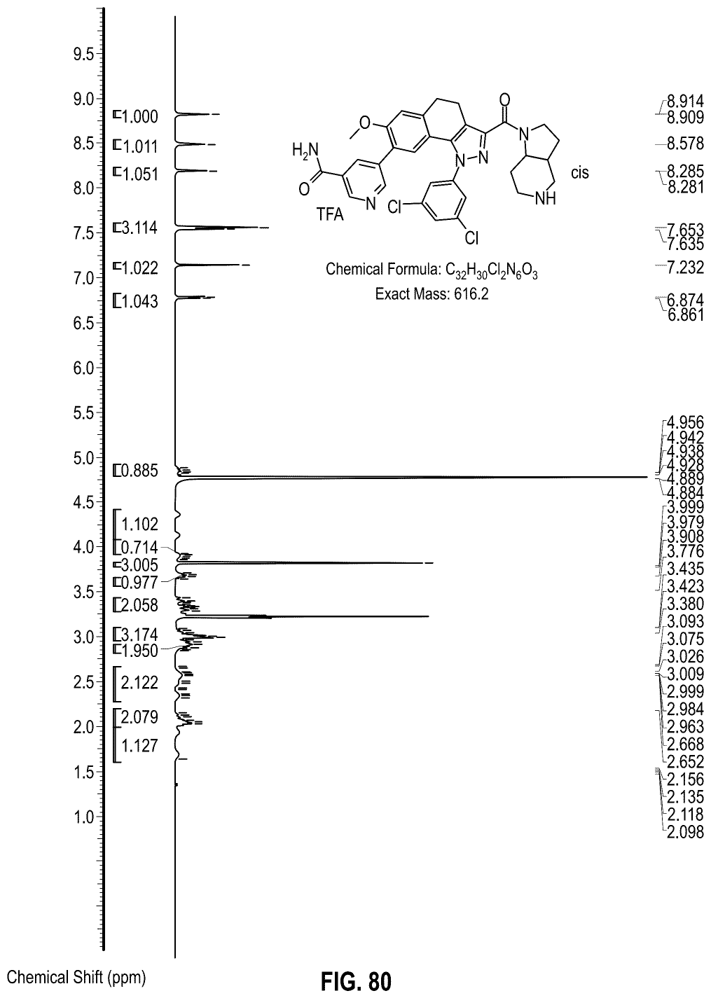

A mixture of tert-butyl 1-[8-(5-carbamoyl-3-pyridyl)-1-(3,5-dichlorophenyl)-7-methoxy-4,5-dihydrobenzo[g]indazole-3-carbonyl]-3,3a,4,6,7,7a-hexahydro-2H-pyrrolo[3,2-c]pyridine-5-carboxylate (20 mg, 27.87 μmol, 1 eq) in 4 N HCl/dioxane (2 mL) was stirred at 15° C. for 1 h. The mixture was concentrated under vacuum to give a residue. The residue was purified by prep-HPLC (TFA condition; or neutral condition; or basic condition) followed by lyophilization to afford 5-[3-(2,3,3a,4,5,6,7,7a-octahydropyrrolo[3,2-c]pyridine-1-carbonyl)-1-(3,5-dichlorophenyl)-7-methoxy-4,5-dihydrobenzo[g]indazol-8-yl]pyridine-3-carboxamide (11.9 mg, 16.27 μmol, 58% yield, 100% purity, TFA) as a white solid. 1H NMR (400 MHz, METHANOL-d4) δ=8.91 (d, J=1.8 Hz, 1H), 8.58 (s, 1H), 8.28 (d, J=1.8 Hz, 1H), 7.70-7.60 (m, 3H), 7.23 (s, 1H), 6.87 (d, J=5.3 Hz, 1H), 5.00-4.87 (m, 1H), 4.51-4.17 (m, 1H), 4.09-3.92 (m, 1H), 3.91 (s, 3H), 3.82-3.72 (m, 1H), 3.33 (br s, 3H), 3.19-3.06 (m, 3H), 3.05-2.93 (m, 2H), 2.76-2.36 (m, 2H), 2.28-2.08 (m, 2H), 2.07-1.69 (m, 1H). LCMS (ESI):m/z [M+H] calcd for C₃₄H₃₁N₆O₅Cl₂F₃: 617.18; found: 617.2. FIG. 80 shows the nuclear magnetic resonance of Compound 8-23.

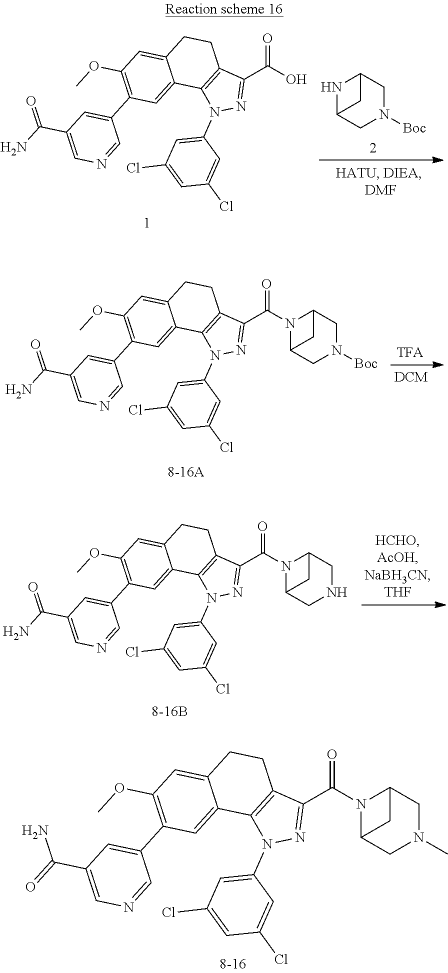 [SMILES: COc1cc2c(cc1-c1cncc(C(N)=O)c1)-c1c(c(C(=O)O)nn1-c1cc(Cl)cc(Cl)c1)CC2 ; CC(C)(C)OC(=O)N1CC2CC(C1)N2 ; COc1cc2c(cc1-c1cncc(C(N)=O)c1)-c1c(c(C(=O)N3C4CC3CN(C(=O)OC(C)(C)C)C4)nn1-c1cc(Cl)cc(Cl)c1)CC2 ; COc1cc2c(cc1-c1cncc(C(N)=O)c1)-c1c(c(C(=O)N3C4CNCC3C4)nn1-c1cc(Cl)cc(Cl)c1)CC2 ; COc1cc2c(cc1-c1cncc(C(N)=O)c1)-c1c(c(C(=O)N3C4CC3CN(C)C4)nn1-c1cc(Cl)cc(Cl)c1)CC2]

### Synthesis of Compound 8-16A

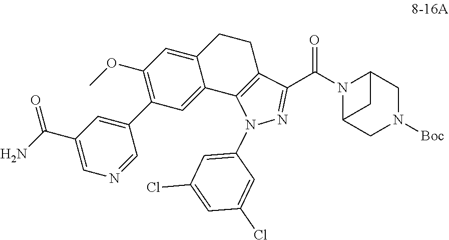 [SMILES: COc1cc2c(cc1-c1cncc(C(N)=O)c1)-c1c(c(C(=O)N3C4CC3CN(C(=O)OC(C)(C)C)C4)nn1-c1cc(Cl)cc(Cl)c1)CC2]

Compound 8-16A was synthesized via a method similar to example 8. LCMS (ESI): m/z [M+H] calcd for C₃₅H₃₄Cl₂N₆O₅: 689.20; found: 689.2. 1H NMR (400 MHz, METHANOL-d4) δ=8.96-8.79 (m, 1H), 8.62-8.44 (m, 1H), 8.22 (br s, 1H), 7.67-7.60 (m, 3H), 7.21 (br s, 1H), 6.82 (br d, J=9.4 Hz, 1H), 5.12 (br d, J=15.8 Hz, 1H), 4.65-4.52 (m, 1H), 4.04-3.91 (m, 2H), 3.90-3.87 (m, 3H), 3.68-3.51 (m, 2H), 3.06 (br d, J=5.0 Hz, 4H), 2.85-2.77 (m, 1H), 1.65 (dd, J=5.9, 8.8 Hz, 1H), 1.46 (d, J=13.2 Hz, 9H).

### Synthesis of Compound 8-16B

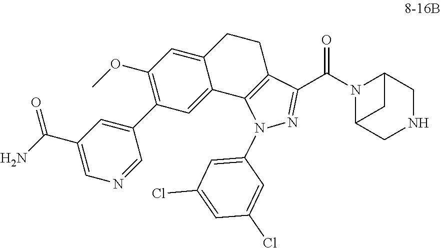 [SMILES: COc1cc2c(cc1-c1cncc(C(N)=O)c1)-c1c(c(C(=O)N3C4CNCC3C4)nn1-c1cc(Cl)cc(Cl)c1)CC2]

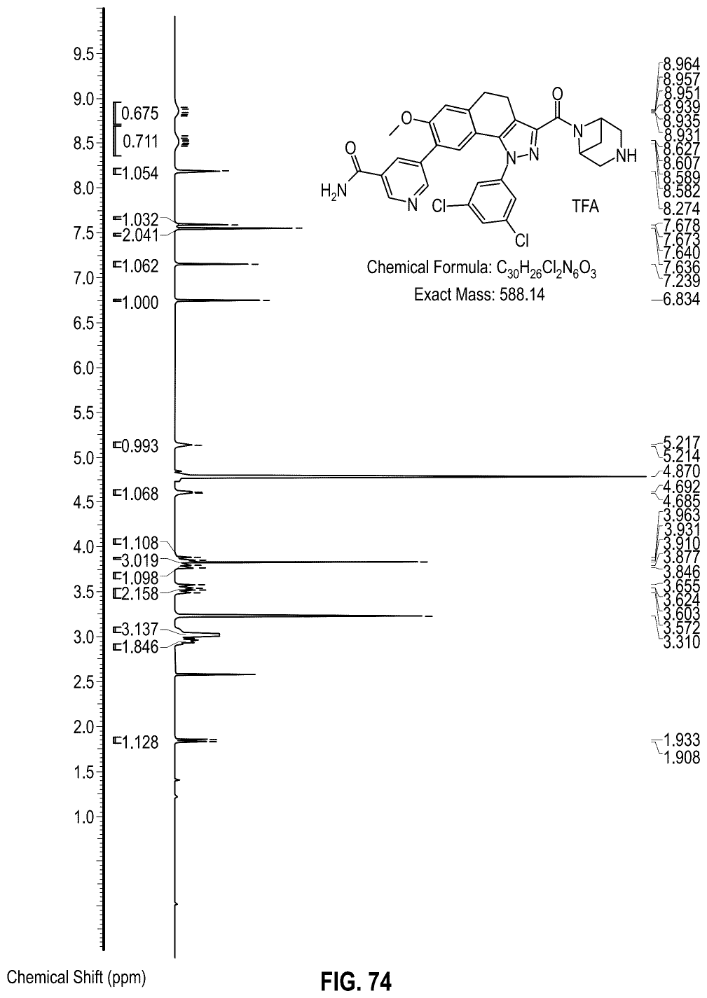

Compound 8-16B was synthesized via a method similar to example 8. LCMS (ESI): m/z [M+H] calcd for C₃₀H₂₇Cl₂N₆O₃: 589.14; found: 589.1. 1H NMR (400 MHz, METHANOL-d4) δ=9.05-8.80 (m, 1H), 8.78-8.44 (m, 1H), 8.27 (s, 1H), 7.68 (d, J=1.7 Hz, 1H), 7.64 (d, J=1.6 Hz, 2H), 7.24 (s, 1H), 6.83 (s, 1H), 5.22 (br d, J=1.5 Hz, 1H), 4.69 (br d, J=3.1 Hz, 1H), 3.95 (br d, J=12.8 Hz, 1H), 3.91 (s, 3H), 3.86 (br d, J=12.3 Hz, 1H), 3.67-3.55 (m, 2H), 3.15-3.07 (m, 3H), 3.06-2.99 (m, 2H), 1.92 (d, J=10.0 Hz, 1H). FIG. 74 shows the nuclear magnetic resonance of Compound 8-16B.

### Synthesis of Compound 8-16

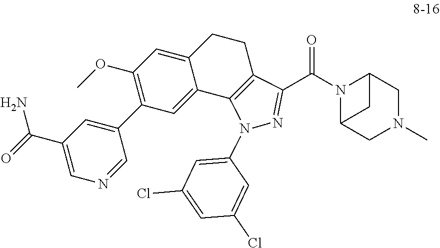 [SMILES: COc1cc2c(cc1-c1cncc(C(N)=O)c1)-c1c(c(C(=O)N3C4CC3CN(C)C4)nn1-c1cc(Cl)cc(Cl)c1)CC2]

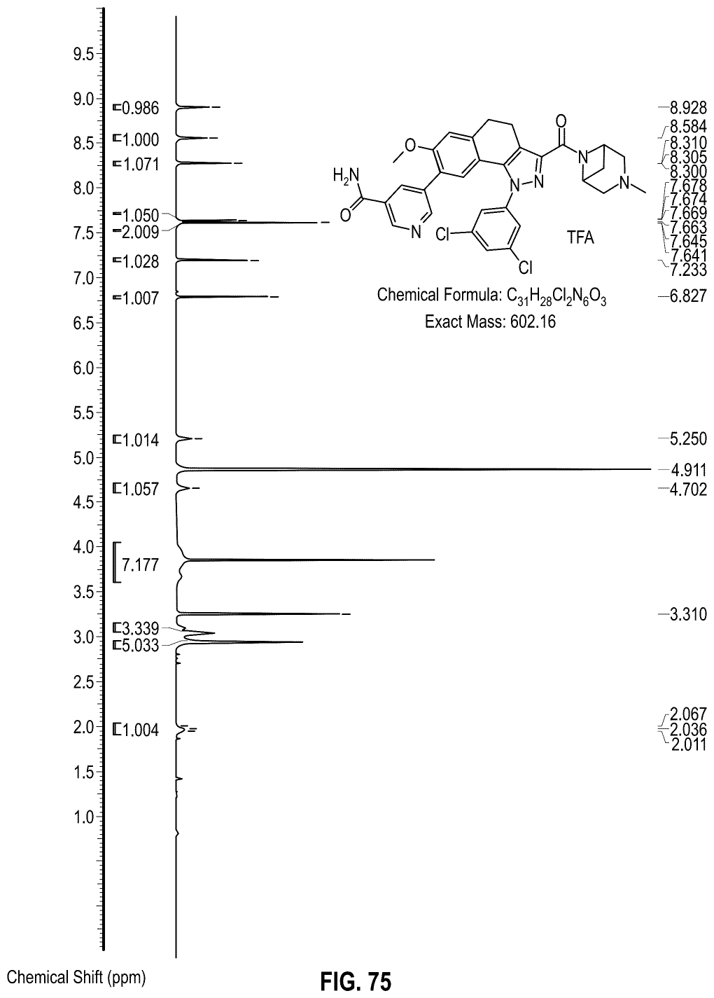

Compound 8-16 was synthesized via a method similar to example 8. LCMS (ESI): m/z [M+H] calcd for C₃₁H₂₉Cl₂N₆O₃: 603.16; found: 603.1. 1H NMR (400 MHz, METHANOL-d4) δ=8.93 (d, J=1.0 Hz, 1H), 8.58 (s, 1H), 8.31 (t, J=2.0 Hz, 1H), 7.68-7.66 (m, 1H), 7.64 (d, J=1.8 Hz, 2H), 7.23 (s, 1H), 6.83 (s, 1H), 5.25 (br s, 1H), 4.70 (br s, 1H), 4.10-3.66 (m, 7H), 3.17-3.06 (m, 3H), 3.05-2.96 (m, 5H), 2.07-1.97 (m, 1H). FIG. 75 shows the nuclear magnetic resonance of Compound 8-16.

### Synthesis of Compound 8-25A

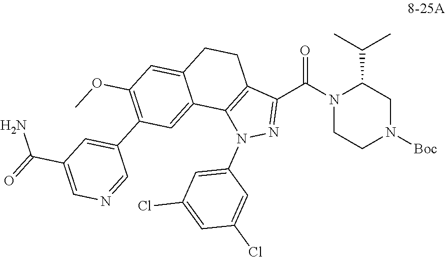 [SMILES: COc1cc2c(cc1-c1cncc(C(N)=O)c1)-c1c(c(C(=O)N3CCN(C(=O)OC(C)(C)C)C[C@H]3C(C)C)nn1-c1cc(Cl)cc(Cl)c1)CC2]

Compound 8-25A was synthesized via a method similar to example 8. LCMS (ESI): m/z [M+H] calcd for C₃₉H₄₁N₆O₇Cl₂F₃: 719.24; found: 719.4. ¹H NMR (400 MHz, METHANOL-d₄) δ=9.61 (d, J=1.7 Hz, 1H), 9.30 (br s, 1H), 9.03 (br s, 1H), 8.31 (br s, 3H), 7.91 (s, 1H), 7.66-7.50 (m, 1H), 5.24-4.90 (m, 3H), 4.89-4.63 (m, 1H), 4.58 (s, 3H), 3.88-3.37 (m, 7H), 2.86-2.71 (m, 1H), 2.14 (s, 9H), 1.82 (br d, J=5.7 Hz, 2H), 1.70-1.59 (m, 3H), 1.54 (br d, J=6.8 Hz, 1H).

### Synthesis of Compound 8-25

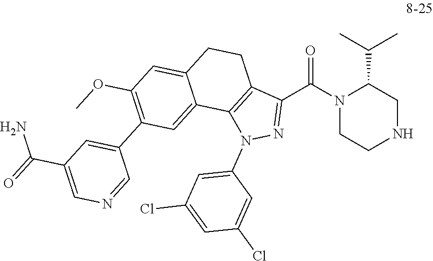 [SMILES: COc1cc2c(cc1-c1cncc(C(N)=O)c1)-c1c(c(C(=O)N3CCNC[C@H]3C(C)C)nn1-c1cc(Cl)cc(Cl)c1)CC2]

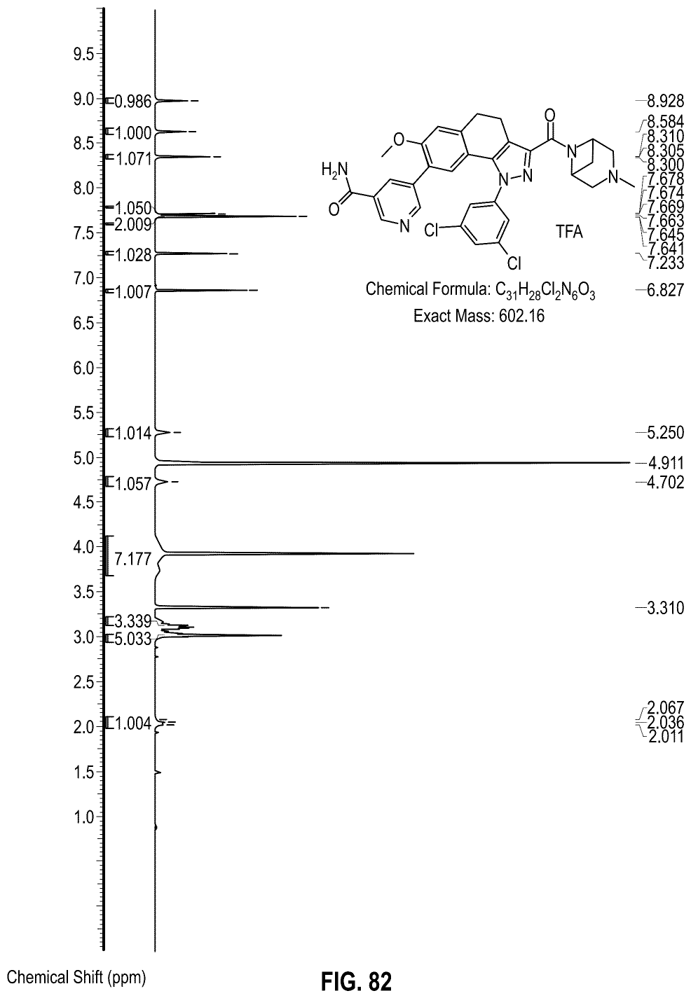

Compound 8-25 was synthesized via a method similar to example 8. LCMS (ESI): m/z [M+H] calcd for C₃₄H₃₃N₆O₅Cl₂F₃: 619.19; found: 619.2. ¹H NMR (400 MHz, METHANOL-d₄) δ=8.91 (s, 1H), 8.58 (br s, 1H), 8.28 (t, J=1.9 Hz, 1H), 7.70-7.56 (m, 3H), 7.24 (s, 1H), 6.87 (br s, 1H), 4.98-4.87 (m, 1H), 4.73-4.57 (m, 1H), 3.91 (s, 3H), 3.76-3.32 (m, 3H), 3.29-3.17 (m, 2H), 3.16-3.05 (m, 2H), 2.93 (br s, 2H), 2.51-2.18 (m, 1H), 1.26-0.76 (m, 6H). FIG. 82 shows the nuclear magnetic resonance of Compound 8-25.

### Synthesis of Compound 8-27

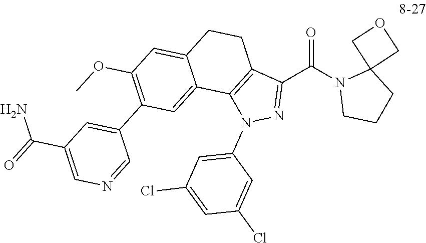 [SMILES: COc1cc2c(cc1-c1cncc(C(N)=O)c1)-c1c(c(C(=O)N3CCCC34COC4)nn1-c1cc(Cl)cc(Cl)c1)CC2]

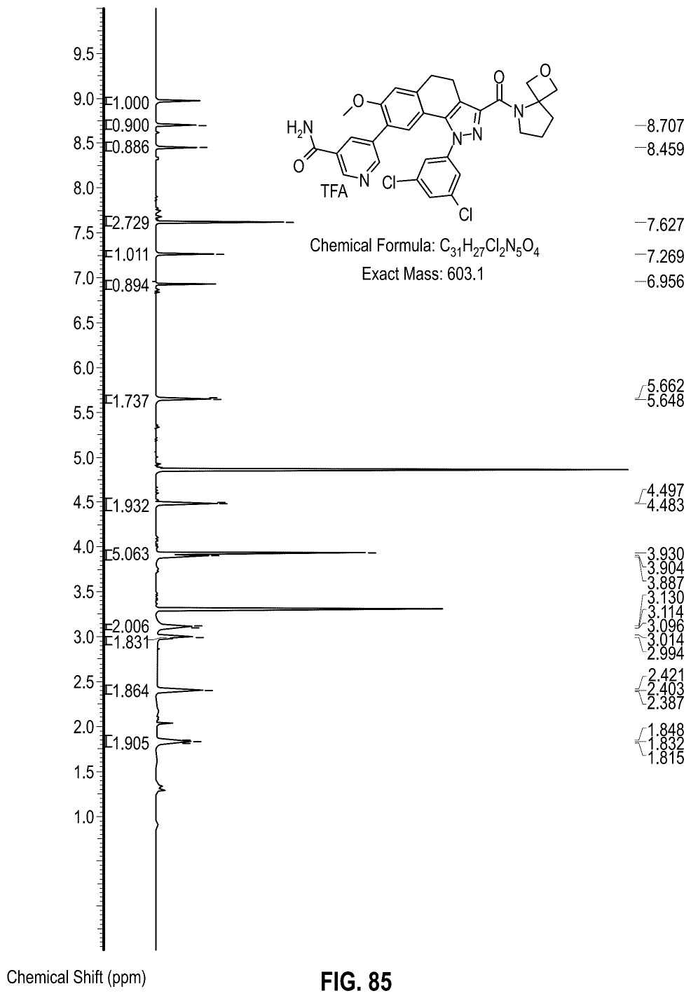

Compound 8-27 was synthesized via a method similar to example 8. LCMS (ESI): m z [M+H] calcd for C₃₃H₂₈N₅O₆Cl₂F₃: 604.1; found: 604.2. ¹H NMR (400 MHz, METHANOL-d₄) δ=8.98 (s, 1H), 8.71 (s, 1H), 8.46 (s, 1H), 7.63 (s, 3H), 7.27 (s, 1H), 6.93 (s, 1H), 5.65 (d, J=5.4 Hz, 2H), 4.49 (d, J=5.5 Hz, 2H), 3.96-3.86 (m, 5H), 3.14-3.06 (m, 2H), 3.04-2.95 (m, 2H), 2.40 (t, J=6.7 Hz, 2H), 1.83 (br t, J=6.4 Hz, 2H). FIG. 85 shows the nuclear magnetic resonance of Compound 8-27.

### Synthesis of Compound 8-31A

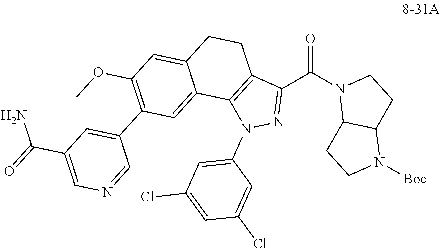 [SMILES: COc1cc2c(cc1-c1cncc(C(N)=O)c1)-c1c(c(C(=O)N3CCC4C3CCN4C(=O)OC(C)(C)C)nn1-c1cc(Cl)cc(Cl)c1)CC2]

Compound 8-31A was synthesized via a method similar to example 8. LCMS (ESI): m z [M+H] calcd for C₃₈H₃₇N₆O₇Cl₂F₃: 703.21; found: 703.2. ¹H NMR (400 MHz, METHANOL-d4) δ=8.95 (d, J=1.7 Hz, 1H), 8.66 (s, 1H), 8.40 (s, 1H), 7.63 (s, 3H), 7.25 (s, 1H), 6.96-6.86 (m, 1H), 5.31 (br d, J=5.9 Hz, 0.25H), 4.83-4.72 (m, 0.75H), 4.52-4.35 (m, 1H), 4.30-4.02 (m, 1H), 3.92 (s, 3H), 3.70 (br t, J=9.2 Hz, 2H), 3.28-3.18 (m, 1H), 3.13-2.84 (m, 4H), 2.34-1.97 (m, 4H), 1.49 (br s, 9H).

### Synthesis of Compound 8-31

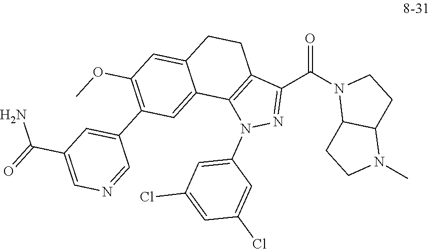 [SMILES: COc1cc2c(cc1-c1cncc(C(N)=O)c1)-c1c(c(C(=O)N3CCC4C3CCN4C)nn1-c1cc(Cl)cc(Cl)c1)CC2]

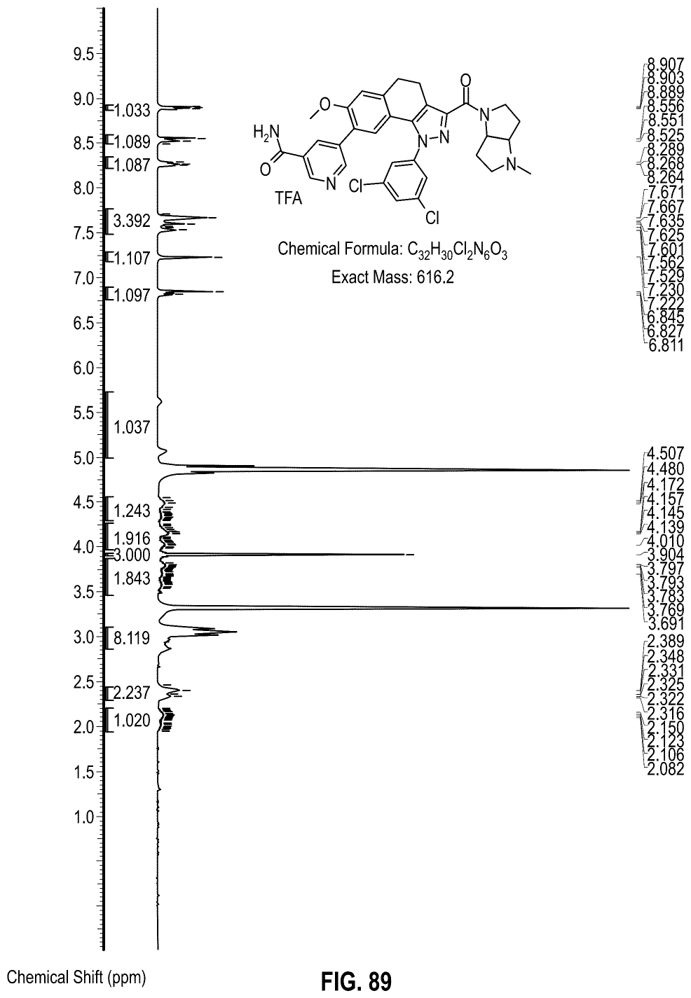

Compound 8-31 was synthesized via a method similar to example 8. LCMS (ESI):m z [M+H] calcd for C₃₄H₃₁N₆O₅Cl₂F₃: 617.18; found: 617.2. ¹H NMR (400 MHz, METHANOL-d4) δ=8.93-8.87 (m, 1H), 8.60-8.49 (m, 1H), 8.34-8.22 (m, 1H), 7.77-7.49 (m, 3H), 7.29-7.18 (m, 1H), 6.89-6.75 (m, 1H), 5.72-4.98 (m, 1H), 4.55-4.28 (m, 1H), 4.26-3.96 (m, 2H), 3.91 (d, J=1.8 Hz, 3H), 3.86-3.45 (m, 2H), 3.10-2.85 (m, 8H), 2.43-2.29 (m, 2H), 2.19-1.93 (m, 1H). FIG. 89 shows the nuclear magnetic resonance of Compound 8-31.

### Synthesis of Compound 8-33A

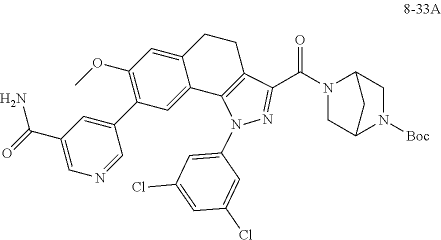 [SMILES: COc1cc2c(cc1-c1cncc(C(N)=O)c1)-c1c(c(C(=O)N3CC4CC3CN4C(=O)OC(C)(C)C)nn1-c1cc(Cl)cc(Cl)c1)CC2]

Compound 8-33A was synthesized via a method similar to example 8. LCMS (ESI): m/z [M+H] calcd for C₃₇H₃₅N₆O₇Cl₂F₃: 689.2; found: 689.2. ¹H NMR (400 MHz, METHANOL-d₄) δ=8.95 (s, 1H), 8.65 (s, 1H), 8.39 (br s, 1H), 7.70-7.60 (m, 3H), 7.25 (d, J=2.3 Hz, 1H), 6.95-6.85 (m, 1H), 5.55-4.98 (m, 1H), 4.61-4.52 (m, 1H), 4.04-3.96 (m, 1H), 3.92 (s, 3H), 3.66-3.44 (m, 3H), 3.15-2.94 (m, 4H), 1.98 (br d, J=10.5 Hz, 2H), 1.48 (br d, J=17.1 Hz, 9H).

### Synthesis of Compound 8-33

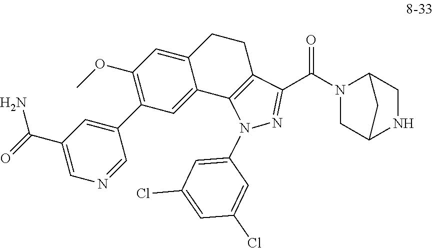 [SMILES: COc1cc2c(cc1-c1cncc(C(N)=O)c1)-c1c(c(C(=O)N3CC4CC3CN4)nn1-c1cc(Cl)cc(Cl)c1)CC2]

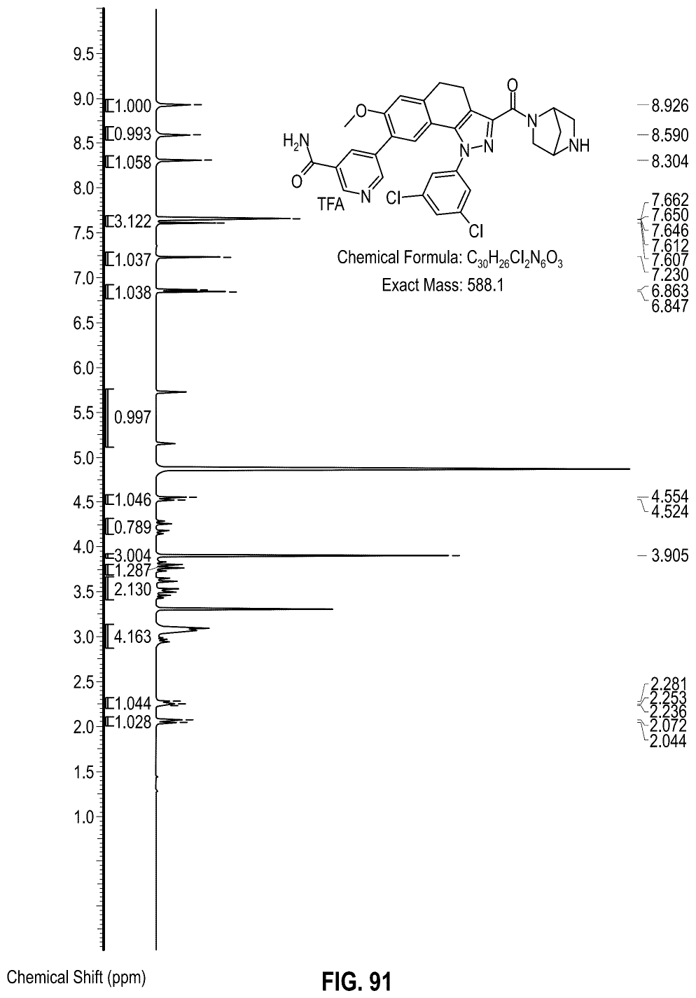

Compound 8-33 was synthesized via a method similar to example 8. LCMS (ESI): m/z [M+H] calcd for C₃₂H₂₇N₆O₅Cl₂F₃: 589.14; found: 589.2. ¹H NMR (400 MHz, METHANOL-d₄) δ=8.93 (s, 1H), 8.59 (br s, 1H), 8.31 (d, J=1.5 Hz, 1H), 7.69-7.57 (m, 3H), 7.23 (s, 1H), 6.92-6.77 (m, 1H), 5.76-5.10 (m, 1H), 4.58-4.48 (m, 1H), 4.32-4.14 (m, 1H), 3.91 (s, 3H), 3.85-3.70 (m, 1H), 3.68-3.39 (m, 2H), 3.14-2.84 (m, 4H), 2.32-2.17 (m, 1H), 2.06 (br d, J=11.4 Hz, 1H). FIG. 91 shows the nuclear magnetic resonance of Compound 8-33.

### Synthesis of Compound 8-34

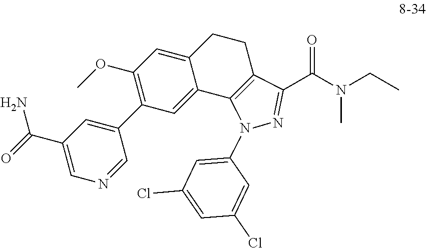 [SMILES: CCN(C)C(=O)c1nn(-c2cc(Cl)cc(Cl)c2)c2c1CCc1cc(OC)c(-c3cncc(C(N)=O)c3)cc1-2]

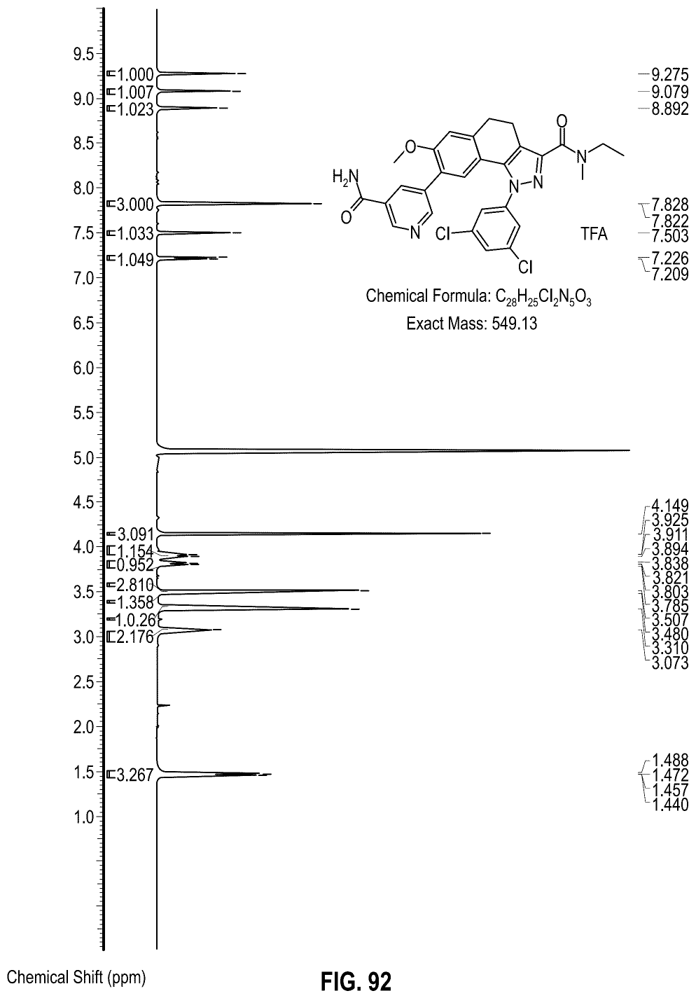

Compound 8-34 was synthesized via a method similar to example 8. LCMS (ESI): m z [M+H] calcd for C₂₈H₂₆Cl₂N₅O₃: 550.13; found: 550.2. 1H NMR (400 MHz, METHANOL-d4) δ=9.27 (s, 1H), 9.08 (s, 1H), 8.89 (br s, 1H), 7.82 (br d, J=2.6 Hz, 3H), 7.50 (s, 1H), 7.22 (d, J=7.0 Hz, 1H), 4.15 (s, 3H), 3.95-3.86 (m, 1H), 3.81 (q, J=7.1 Hz, 1H), 3.51 (s, 3H), 3.48 (s, 1H), 3.35-3.32 (m, 1H), 3.07 (br s, 2H), 1.46 (q, J=6.5 Hz, 3H). FIG. 92 shows the nuclear magnetic resonance of Compound 8-34.

### Synthesis of Compound 8-39

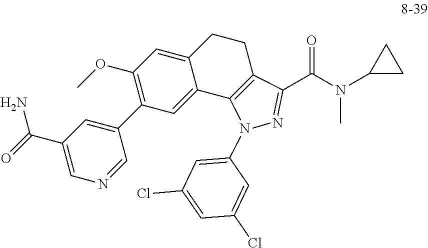 [SMILES: COc1cc2c(cc1-c1cncc(C(N)=O)c1)-c1c(c(C(=O)N(C)C3CC3)nn1-c1cc(Cl)cc(Cl)c1)CC2]

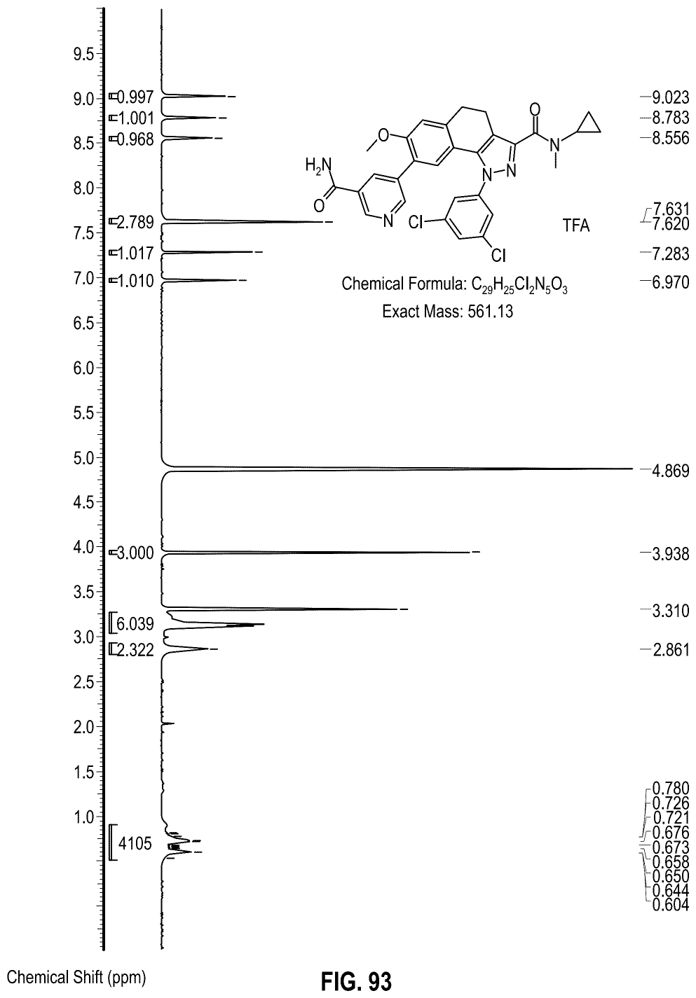

Compound 8-39 was synthesized via a method similar to example 8. LCMS (ESI): m/z [M+H] calcd for C₂₉H₂₆Cl₂N₅O₃: 562.13; found: 562.2. 1H NMR (400 MHz, METHANOL-d4) δ=9.02 (s, 1H), 8.78 (br s, 1H), 8.56 (br s, 1H), 7.66-7.60 (m, 3H), 7.28 (s, 1H), 6.97 (s, 1H), 3.94 (s, 3H), 3.26-3.07 (m, 6H), 2.86 (br s, 2H), 0.82-0.51 (m, 4H). FIG. 93 shows the nuclear magnetic resonance of Compound 8-39.

### Synthesis of Compound 8-44

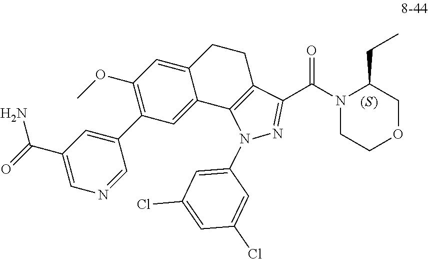

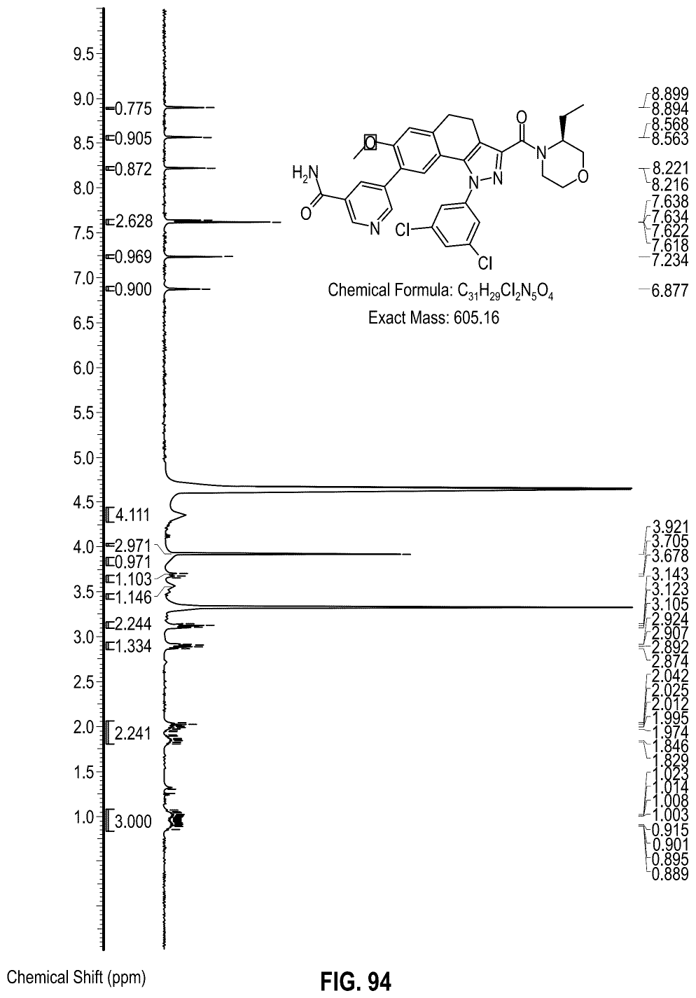

Compound 8-44 was synthesized via a method similar to example 8. LCMS (ESI): m/z [M+H] calcd for C₃₁H₂₉Cl₂N₅O₄: 606.16; found: 606.2. 1H NMR (400 MHz, METHANOL-d4) δ=8.90 (d, J=2.0 Hz, 1H), 8.57 (d, J=2.0 Hz, 1H), 8.22 (t, J=2.1 Hz, 1H), 7.65-7.59 (m, 3H), 7.23 (s, 1H), 6.88 (s, 1H), 4.44-4.26 (m, 4H), 3.92 (s, 3H), 3.89-3.80 (m, 1H), 3.73-3.66 (m, 1H), 3.60-3.53 (m, 1H), 3.17-3.09 (m, 2H), 2.94-2.86 (m, 2H), 2.06-1.81 (m, 2H), 1.08-0.84 (m, 3H). FIG. 94 shows the nuclear magnetic resonance of Compound 8-44.

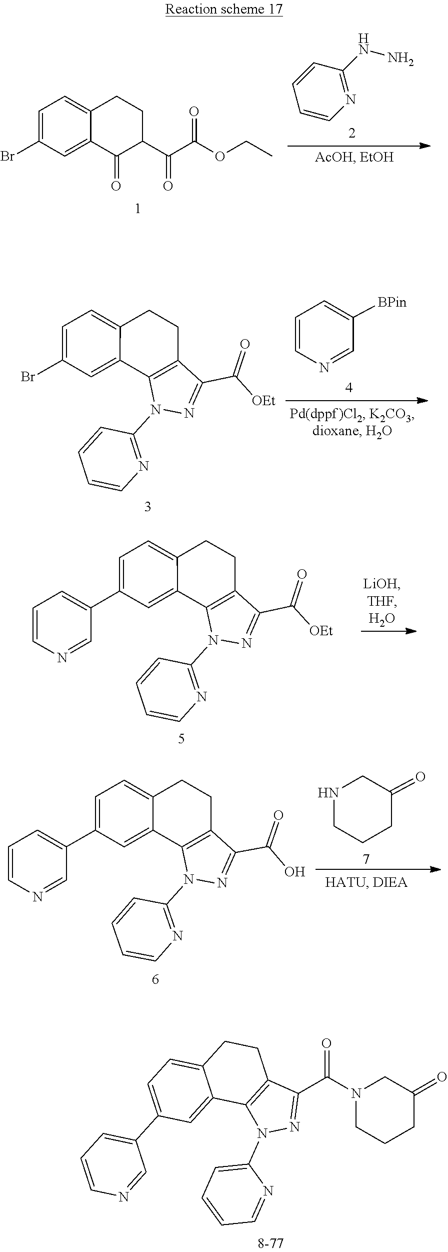

### Synthesis of ethyl 8-bromo-1-(pyridin-2-yl)-4,5-dihydro-1H-benzo[g]indazole-3-carboxylate

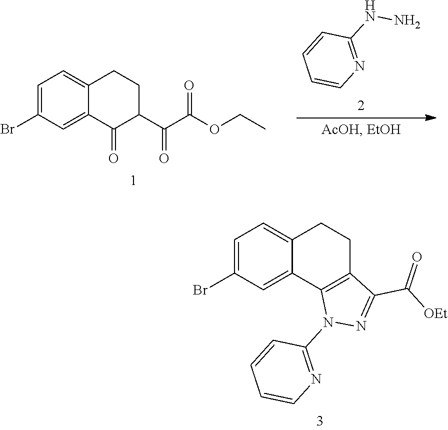

A mixture of ethyl 2-(7-bromo-1-oxo-1,2,3,4-tetrahydronaphthalen-2-yl)-2-oxoacetate (1.9 g, 6.39 mmol, 1 eq), 2-pyridylhydrazine (697.79 mg, 6.39 mmol, 1 eq), AcOH (3.84 g, 63.94 mmol, 3.66 mL, 10 eq) in EtOH (20 mL) was degassed and purged with N₂ for 3 times, and then the mixture was stirred at 80° C. for 16 hr under N₂ atmosphere. The mixture was poured into water (80 mL) and extracted with ethyl acetate (3*40 mL). The organic phase was separated, washed with Saturated sodium chloride solution (20 mL), dried over Na2SO4, filtered and concentrated under reduced pressure to give a residue. The residue was purified by flash silica gel chromatography (ISCO®; 25 g SepaFlash® Silica Flash Column, Eluent of 60˜40% Petroleum ether/Ethyl acetate gradient @60 mL/min). Compound ethyl 8-bromo-1-(pyridin-2-yl)-4,5-dihydro-1H-benzo[g]indazole-3-carboxylate (1.1 g, 2.73 mmol, 42.76% yield, 99% purity) was obtained as a brown solid. 1H NMR (400 MHz, DMSO-d6) δ=8.57 (d, J=4.0 Hz, 1H), 8.19 (dt, J=1.6, 8.0 Hz, 1H), 7.84 (d, J=8.0 Hz, 1H), 7.66 (m, J=5.1, 1H), 7.45-7.38 (m, 1H), 7.37-7.31 (m, 1H), 6.79 (d, J=1.6 Hz, 1H), 4.34 (q, J=7.2 Hz, 2H), 3.03-2.88 (m, 4H), 1.33 (t, J=7.2 Hz, 3H).

### Synthesis of ethyl 8-bromo-1-(2-pyridyl)-4,5-dihydrobenzo[g]indazole-3-carboxylate

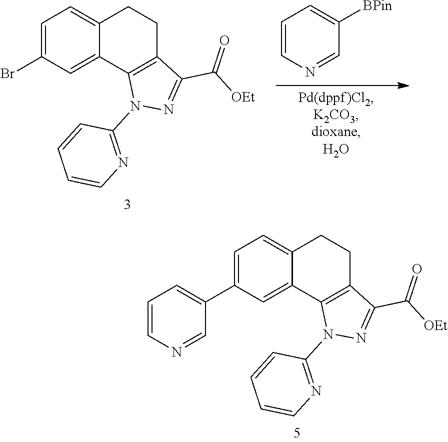

A mixture of ethyl 8-bromo-1-(2-pyridyl)-4,5-dihydrobenzo[g]indazole-3-carboxylate (900 mg, 2.26 mmol, 1 eq), 3-pyridylboronic acid (333.33 mg, 2.71 mmol, 1.2 eq), Pd(dppf)Cl2 (165.36 mg, 225.99 μmol, 0.1 eq), K₂C₀₃ (936.98 mg, 6.78 mmol, 3 eq) in dioxane (10 mL) and H2O (1 mL) was degassed and purged with N2 for 3 times, and then the mixture was stirred at 60° C. for 16 hr under N2 atmosphere. The mixture was filtered and concentrated under reduced pressure to give a residue. The residue was purified by flash silica gel chromatography (ISCO®; 25 g SepaFlash® Silica Flash Column, Eluent of 100˜95% Petroleum ether/Ethyl acetategradient @60 mL/min). Compound ethyl 8-bromo-1-(2-pyridyl)-4,5-dihydrobenzo[g]indazole-3-carboxylate (530 mg, 1.33 mmol, 58.89% yield) was obtained as a gray solid. LCMS (ESI): m/z[M+H] calcd for C19H16N3O2Br: 398.25; found: 397.1.

### Synthesis of 1-(pyridin-2-yl)-8-(pyridin-3-yl)-4,5-dihydro-1H-benzo[g]indazole-3-carboxylic acid

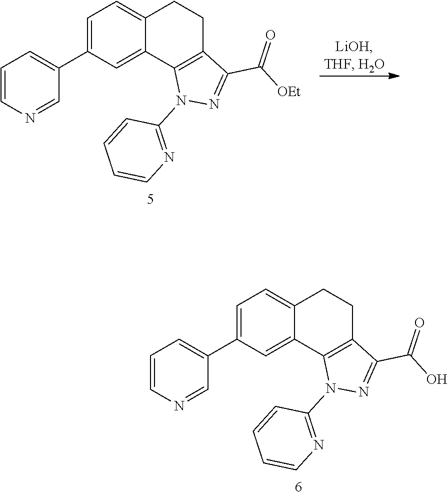

To a solution of ethyl 1-(2-pyridyl)-8-(3-pyridyl)-4,5-dihydrobenzo[g]indazole-3-carboxylate (360 mg, 908.08 μmol, 1 eq) in THF (2 mL) and H2O (2 mL) was added LiOH·H2O (152.43 mg, 3.63 mmol, 4 eq). The mixture was stirred at 25° C. for 2 hr. The mixture was poured into water (40 mL) and extracted with ethyl acetate (3*20 mL). The organic phase was separated, washed with Saturated sodium chloride solution (10 mL), drie d over Na2SO4, filtered and concentrated under reduced pressure to give a residue. Compound 1-(2-pyridyl)-8-(3-pyridyl)-4,5-dihydrobenzo[g]indazole-3-carboxylic acid (240 mg, 651.49 μmol, 71.74% yield) was obtained as a gray solid.

### Synthesis of Compound 8-77

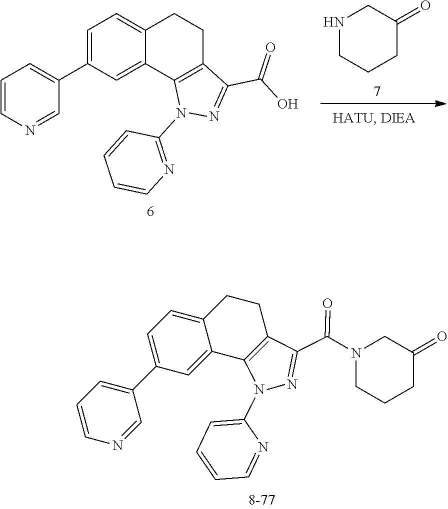 [SMILES: O=C(O)c1nn(-c2ccccn2)c2c1CCc1ccc(-c3cccnc3)cc1-2 ; O=C1CCCNC1 ; O=C1CCCN(C(=O)c2nn(-c3ccccn3)c3c2CCc2ccc(-c4cccnc4)cc2-3)C1]

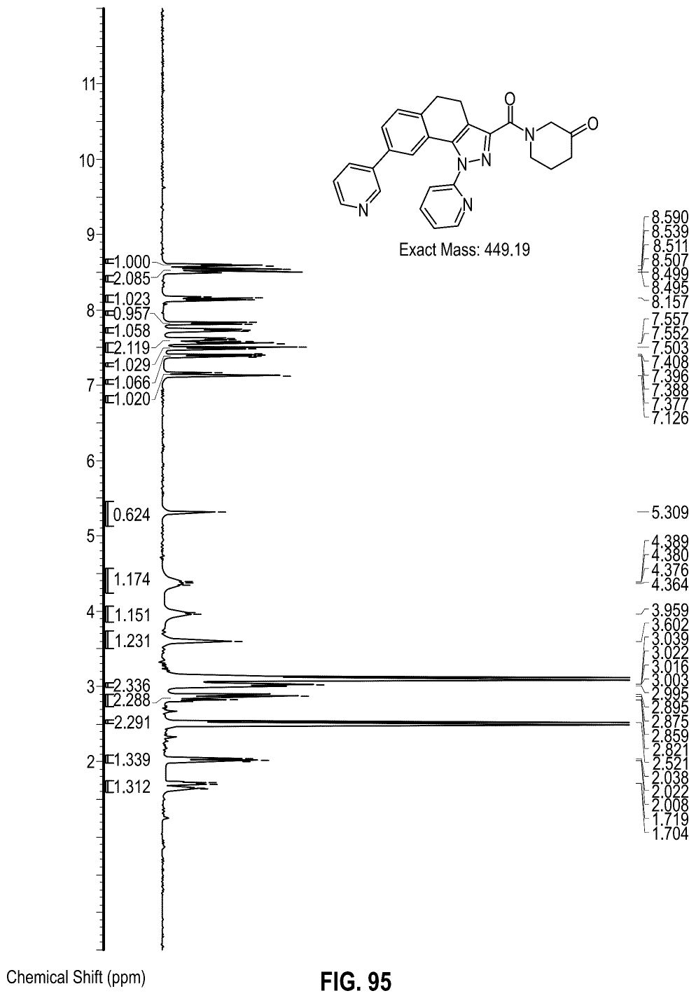

To a solution of 1-(2-pyridyl)-8-(3-pyridyl)-4,5-dihydrobenzo[g]indazole-3-carboxylic acid (40 mg, 108.58 μmol, 1 eq) in DMF (1 mL) was added HATU (61.93 mg, 162.87 μmol, 1.5 eq) and DIEA (42.10 mg, 325.74 μmol, 56.74 μL, 3 eq). The mixture was stirred at 25° C. for 0.5 hr. Then piperidin-3-one (12.92 mg, 130.30 μmol, 1.2 eq) was added. The mixture was stirred at 25° C. for 1.5 hr. The residue was purified by prep-HPLC (neutral condition;column: Waters Xbridge 150*25 mm*5 um;mobile phase: [water(NH4HCO3)-ACN];gradient:18%-48% B over 15 min). Compound 1-[1-(2-pyridyl)-8-(3-pyridyl)-4,5-dihydrobenzo[g]indazole-3-carbonyl]piperidin-3-one (38.29 mg, 84.33 μmol, 77.67% yield, 99% purity) was obtained as a brown oil. LCMS (ESI): m/z[M+H] calcd for C27H24O2N5: 450.19; found: 450.3. ¹H NMR (400 MHz, DMSO-d6) δ=8.63-8.57 (m, 1H), 8.56-8.48 (m, 2H), 8.19-8.11 (m, 1H), 7.85-7.79 (m, 1H), 7.77-7.69 (m, 1H), 7.65-7.53 (m, 2H), 7.52-7.47 (m, 1H), 7.39 (m, 1H), 7.19-7.09 (m, 1H), 5.31 (s, 1H), 4.56-4.24 (m, 1H), 4.06-3.85 (m, 1H), 3.60 (s, 1H), 3.05-2.99 (m, 2H), 2.93-2.77 (m, 2H), 2.52 (s, 2H), 2.09-1.98 (m, 1H), 1.75-1.59 (m, 1H). FIG. 95 shows the nuclear magnetic resonance of Compound 8-77.

### Synthesis of Compound 8-75

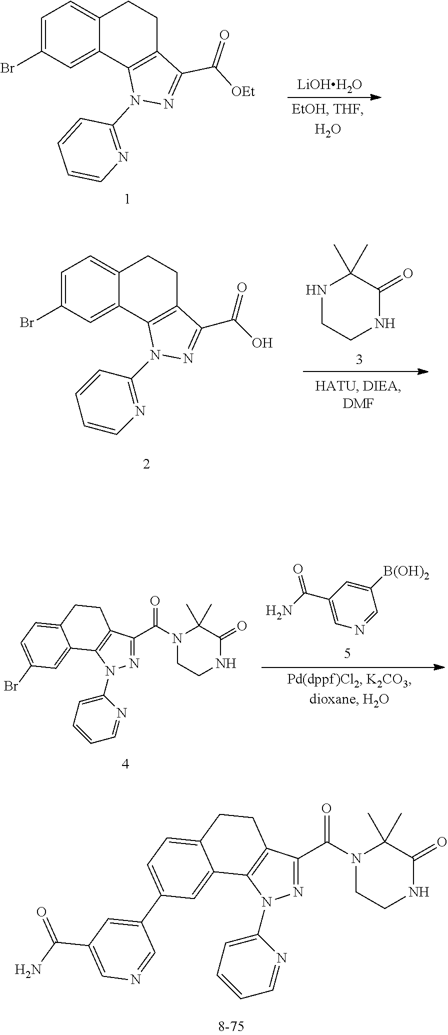

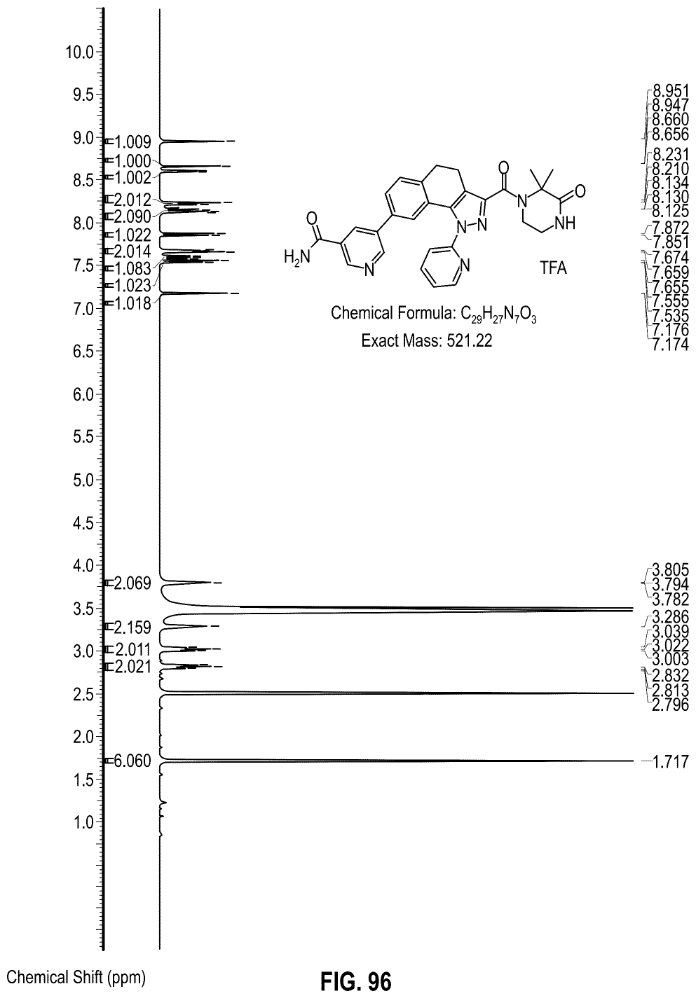

Compound 8-75 was synthesized via a method similar to example 8. LCMS (ESI): m/z [M+H] calcd for C₃₁H₂₉N₇O₅F₃: 522.22; found: 522.2. ¹H NMR (400 MHz, DMSO-d₆) δ=8.95 (d, J=1.5 Hz, 1H), 8.66 (d, J=1.7 Hz, 1H), 8.62-8.58 (m, 1H), 8.27-8.19 (m, 2H), 8.18-8.10 (m, 2H), 7.86 (d, J=8.1 Hz, 1H), 7.70-7.63 (m, 2H), 7.59 (dd, J=5.0, 7.4 Hz, 1H), 7.55 (d, J=7.9 Hz, 1H), 7.18 (d, J=1.1 Hz, 1H), 3.83-3.76 (m, 2H), 3.29 (br s, 2H), 3.06-2.98 (m, 2H), 2.86-2.78 (m, 2H), 1.72 (s, 6H). FIG. 96 shows the nuclear magnetic resonance of Compound 8-75.

### Synthesis of Compound 8-76

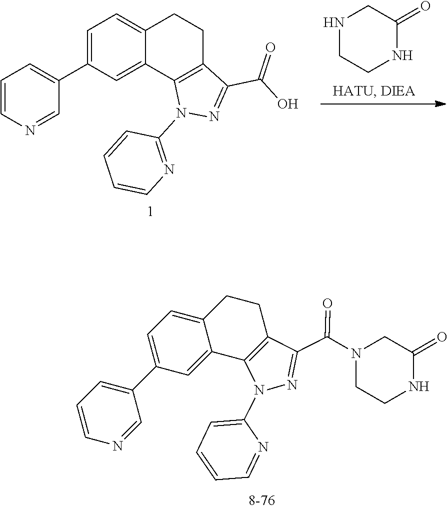 [SMILES: O=C(O)c1nn(-c2ccccn2)c2c1CCc1ccc(-c3cccnc3)cc1-2 ; O=C1CNCCN1 ; O=C1CN(C(=O)c2nn(-c3ccccn3)c3c2CCc2ccc(-c4cccnc4)cc2-3)CCN1]

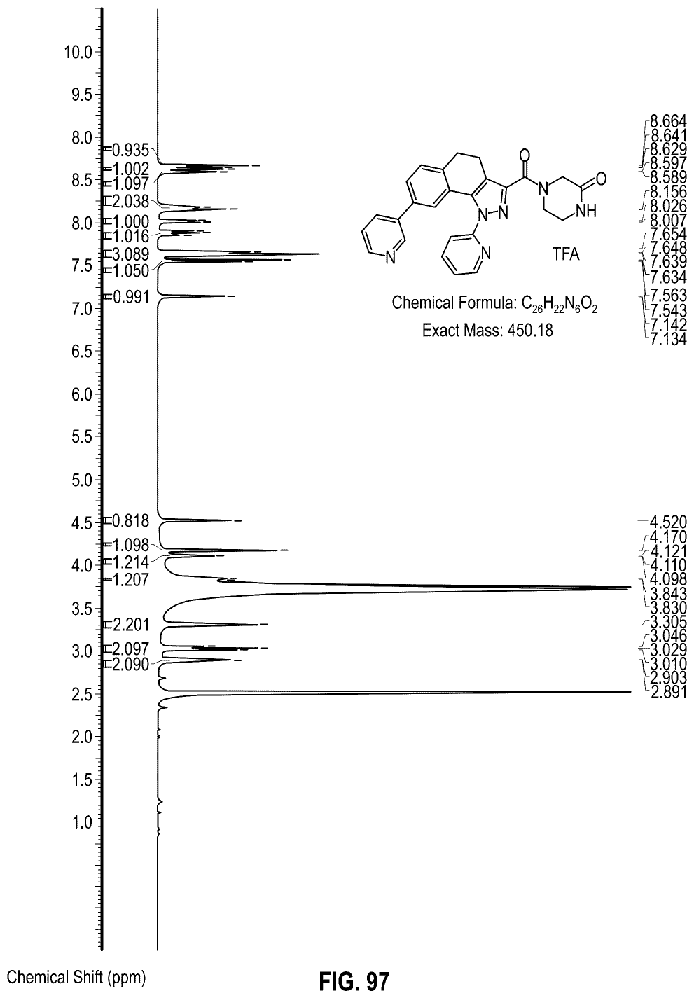

Compound 8-76 was synthesized via a method similar to example 8. LCMS (ESI): m/z[M+H] calcd for C₂₈H₂₄N₆O₄F₃: 451.18; found: 451.1. ¹H NMR (400 MHz, DMSO-d₆) δ=8.67 (d, J=1.3 Hz, 1H), 8.63 (br d, J=4.8 Hz, 1H), 8.59 (br d, J=3.4 Hz, 1H), 8.22-8.12 (m, 2H), 8.02 (br d, J=7.4 Hz, 1H), 7.92-7.85 (m, 1H), 7.68-7.60 (m, 3H), 7.55 (d, J=8.0 Hz, 1H), 7.14 (br d, J=3.1 Hz, 1H), 4.52 (s, 1H), 4.17 (s, 1H), 4.11 (br t, J=4.7 Hz, 1H), 3.84 (br d, J=5.5 Hz, 1H), 3.30 (br s, 2H), 3.06-2.98 (m, 2H), 2.90 (br d, J=4.9 Hz, 2H). FIG. 97 shows the nuclear magnetic resonance of Compound 8-76.

### Synthesis of Compound 8-78

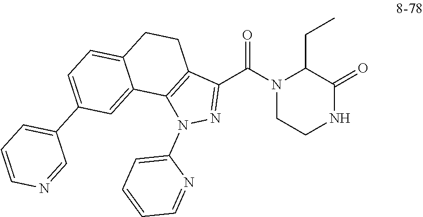 [SMILES: CCC1C(=O)NCCN1C(=O)c1nn(-c2ccccn2)c2c1CCc1ccc(-c3cccnc3)cc1-2]

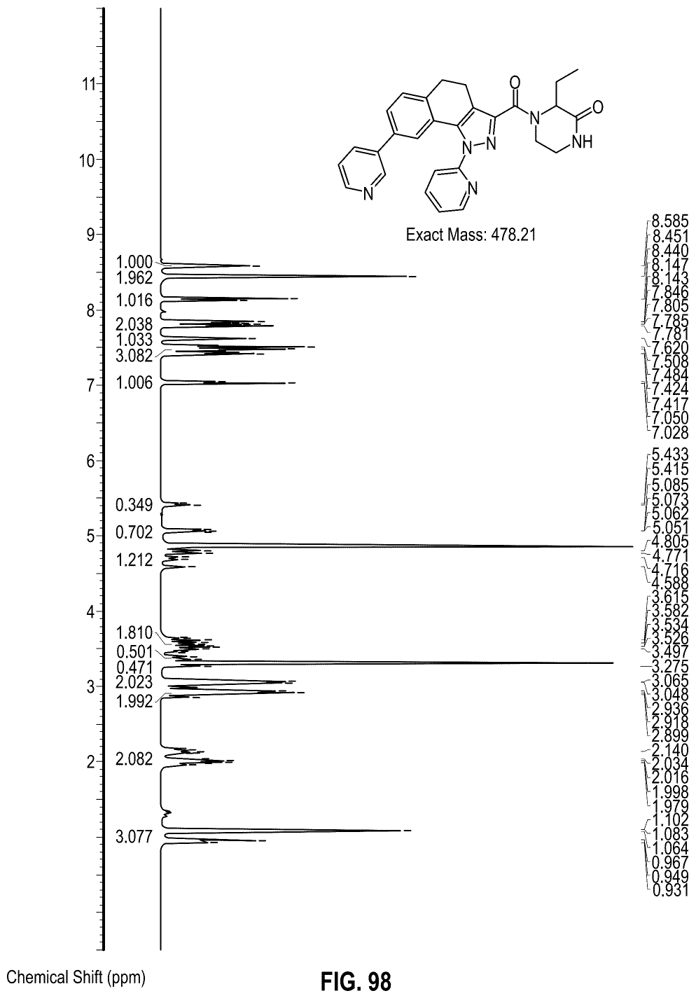

Compound 8-78 was synthesized via a method similar to example 8. LCMS (ESI): m/z[M+H] calcd for C₂₈H₂₆N₆O₂ 479.21; found: 479.3. ¹H NMR (400 MHz, METHANOL-d4) δ=8.59 (d, J=4.4 Hz, 1H), 8.45 (d, J=4.4 Hz, 2H), 8.18-8.10 (m, 1H), 7.88-7.75 (m, 2H), 7.67-7.58 (m, 1H), 7.55-7.38 (m, 3H), 7.08-7.00 (m, 1H), 5.42 (t, J=7.2 Hz, 1H), 5.07 (dd, J=4.8, 9.2 Hz, 1H), 4.83-4.54 (m, 1H), 3.70-3.42 (m, 2H), 3.38 (d, J=14.0 Hz, 1H), 3.28 (s, 1H), 3.06 (d, J=6.8 Hz, 2H), 2.97-2.83 (m, 2H), 2.22-1.88 (m, 2H), 1.15-0.90 (m, 3H). FIG. 98 shows the nuclear magnetic resonance of Compound 8-78.

### Synthesis of Compound 8-81

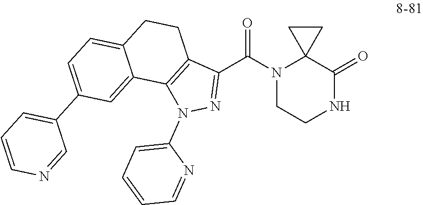 [SMILES: O=C(c1nn(-c2ccccn2)c2c1CCc1ccc(-c3cccnc3)cc1-2)N1CCNC(=O)C12CC2]

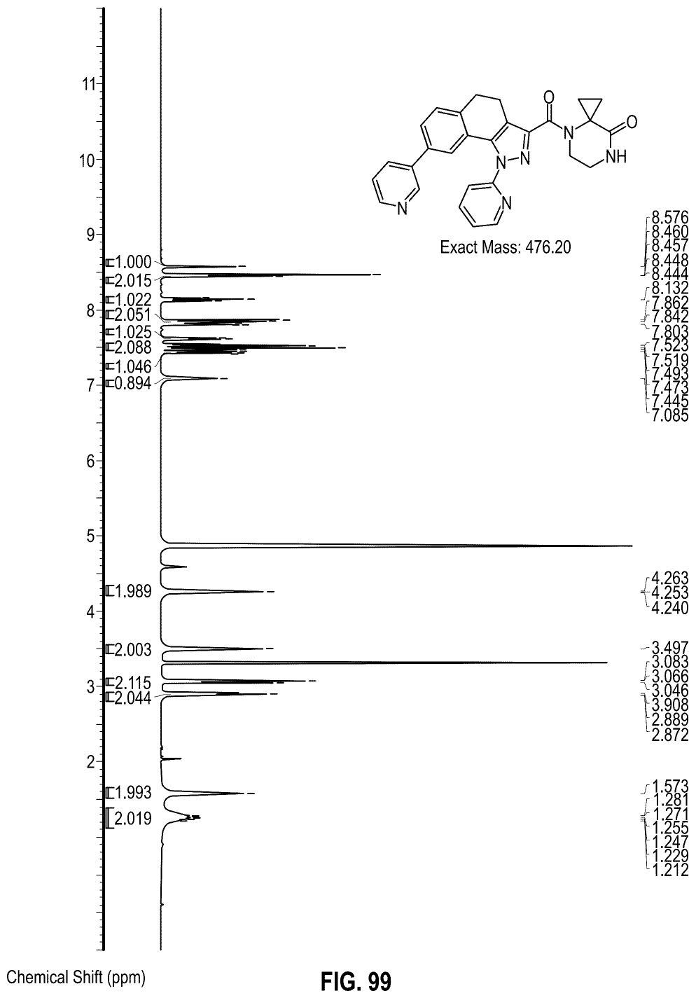

Compound 8-81 was synthesized via a method similar to example 8. LCMS (ESI): m/z[M+H] calcd for C28H24N6O2: 477.2; found: 477.2. ¹H NMR (400 MHz, METHANOL-d4) δ=8.61-8.53 (m, 1H), 8.50-8.41 (m, 2H), 8.13 (dt, J=1.6, 8.0 Hz, 1H), 7.88-7.78 (m, 2H), 7.61 (dd, J=4.8, 7.6 Hz, 1H), 7.56-7.46 (m, 2H), 7.43 (dd, J=5.2, 8.0 Hz, 1H), 7.08 (s, 1H), 4.25 (t, J=4.8 Hz, 2H), 3.50 (s, 2H), 3.11-3.01 (m, 2H), 2.95-2.83 (m, 2H), 1.57 (s, 2H), 1.38-1.12 (m, 2H). FIG. 99 shows the nuclear magnetic resonance of Compound 8-81.

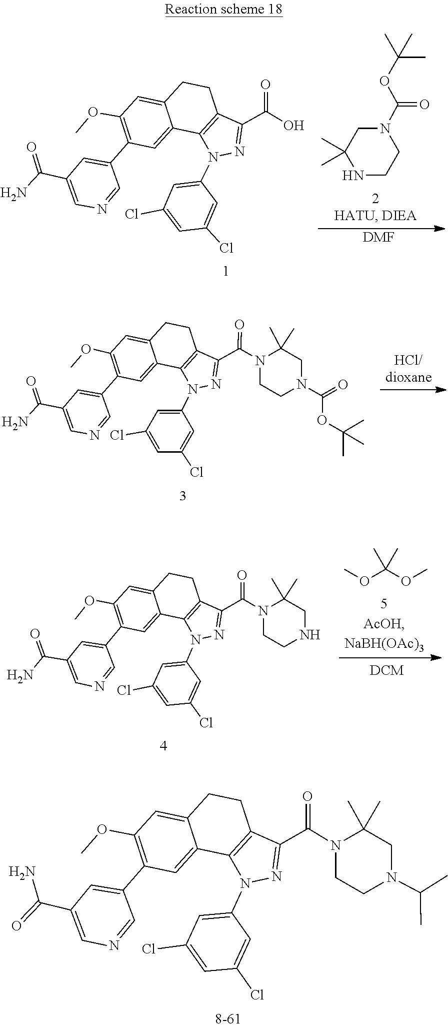

### Synthesis of tert-butyl 4-(8-(5-carbamoylpyridin-3-yl)-1-(3,5-dichlorophenyl)-7-methoxy-4,5-dihydro-1H-benzo[g]indazole-3-carbonyl)-3,3-dimethylpiperazine-1-carboxylate

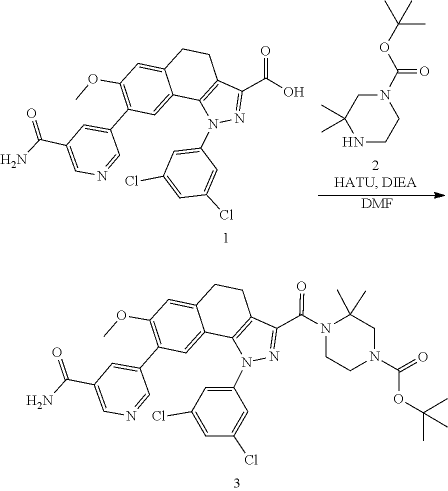

To a solution of 8-(5-carbamoylpyridin-3-yl)-1-(3,5-dichlorophenyl)-7-methoxy-4,5-dihydro-1H-benzo[g]indazole-3-carboxylic acid (500 mg, 981.66 μmol, 1 eq) in DMF (5 mL) was added DIEA (380.62 mg, 2.94 mmol, 512.96 μL, 3 eq) and HATU (559.89 mg, 1.47 mmol, 1.5 eq) at 25° C. After addition. The mixture was stirred at this temperature for 0.5 hr, and then tert-butyl 3,3-dimethylpiperazine-1-carboxylate (252.45 mg, 1.18 mmol, 1.2 eq) was added dropwise at 25° C. The mixture was stirred at 25° C. for 15.5 hr. The reaction mixture was diluted with water (20 mL) and extracted with EtOAc (20 mL). The organic phase was separated, washed with brine (10 mL), dried over Na2SO4, filtered, and concentrated under vacuum to give a residue. The residue was purified by flash silica gel chromatography (ISCO®; 25 g SepaFlash® Silica Flash Column, Eluent of 85% Ethylacetate/Petroleum ethergradient @30 mL/min). Compound tert-butyl 4-(8-(5-carbamoylpyridin-3-yl)-1-(3,5-dichlorophenyl)-7-methoxy-4,5-dihydro-1H-benzo[g]indazole-3-carbonyl)-3,3-dimethylpiperazine-1-carboxylate (160 mg, 226.75 μmol, 23.10% yield) was obtained as a yellow oil. LCMS (ESI): m/z[M+H] calcd for C36H39N6O5Cl2: 705.23; found: 705.4.

### Synthesis of 5-(1-(3,5-dichlorophenyl)-3-(2,2-dimethylpiperazine-1-carbonyl)-7-methoxy-4,5-dihydro-1H-benzo[g]indazol-8-yl)nicotinamide

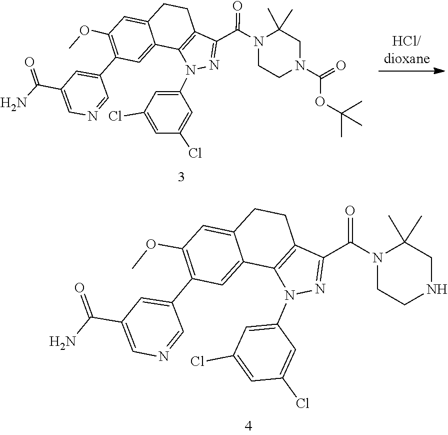 [SMILES: COc1cc2c(cc1-c1cncc(C(N)=O)c1)-c1c(c(C(=O)N3CCN(C(=O)OC(C)(C)C)CC3(C)C)nn1-c1cc(Cl)cc(Cl)c1)CC2 ; COc1cc2c(cc1-c1cncc(C(N)=O)c1)-c1c(c(C(=O)N3CCNCC3(C)C)nn1-c1cc(Cl)cc(Cl)c1)CC2]

To a solution of tert-butyl 4-(8-(5-carbamoylpyridin-3-yl)-1-(3,5-dichlorophenyl)-7-methoxy-4,5-dihydro-1H-benzo[g]indazole-3-carbonyl)-3,3-dimethylpiperazine-1-carboxylate (160 mg, 226.75 μmol, 1 eq) in dioxane (2 mL) was added HCl/dioxane (4 M, 2 mL, 35.28 eq). The mixture was stirred at 25° C. for 1 hr. The reaction mixture was concentrated in vacuum to give residue. Compound 5-(1-(3,5-dichlorophenyl)-3-(2,2-dimethylpiperazine-1-carbonyl)-7-methoxy-4,5-dihydro-1H-benzo[g]indazol-8-yl)nicotinamide (145 mg, 225.87 μmol, 99.61% yield, HCl) was obtained as a yellow solid. LCMS (ESI): m z[M+H] calcd for C₃₁H₃₂Cl₃N₆O₃: 605.18; found: 605.2 ¹H NMR (400 MHz, DMSO-d₆) δ=9.98-9.88 (m, 1H), 9.73-9.59 (m, 1H), 9.43-9.31 (m, 1H), 8.96 (d, J=1.8 Hz, 1H), 8.57-8.51 (m, 1H), 8.22-8.14 (m, 1H), 7.82-7.66 (m, 2H), 7.32 (s, 1H), 6.84 (s, 1H), 3.95-3.89 (m, 2H), 3.86 (s, 3H), 3.32-3.18 (m, 6H), 3.08-3.00 (m, 1H), 2.86-2.79 (m, 1H), 1.59 (s, 3H), 1.45 (s, 3H)

### Synthesis of Compound 8-61

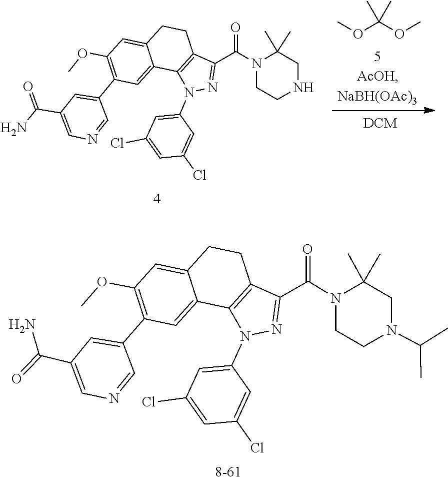

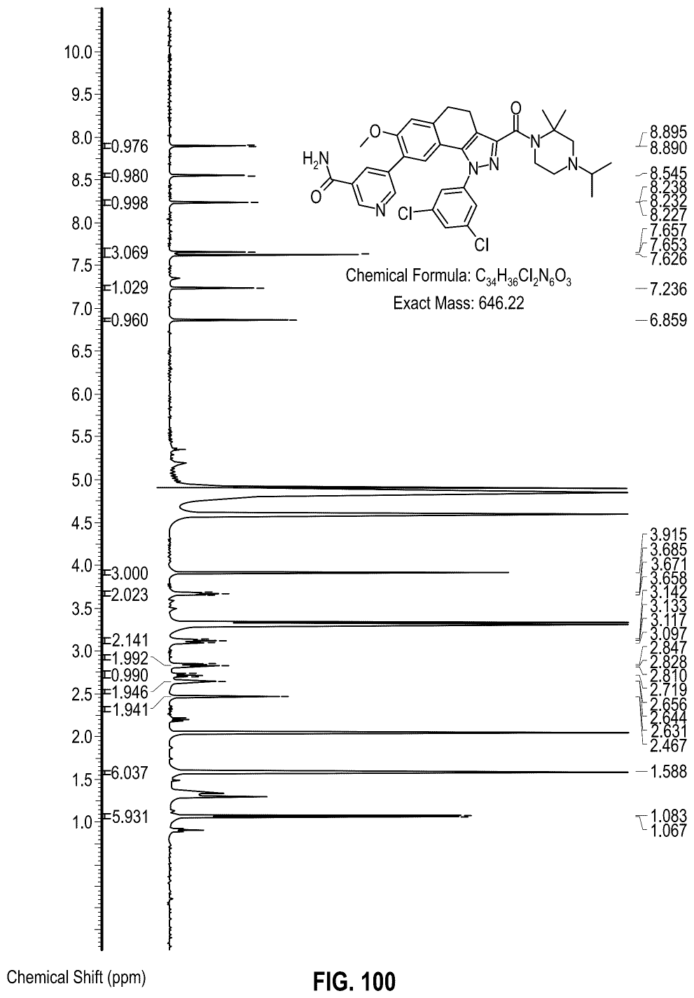

To a solution of 5-(1-(3,5-dichlorophenyl)-3-(2,2-dimethylpiperazine-1-carbonyl)-7-methoxy-4,5-dihydro-1H-benzo[g]indazol-8-yl)nicotinamide (40 mg, 62.31 μmol, 1 eq, HCl) in DCM (3 mL) was added dropwise AcOH (37.42 mg, 623.08 μmol, 35.67 μL, 10 eq) and 2,2-dimethoxypropane (7.79 mg, 74.77 μmol, 9.16 μL, 1.2 eq) at 25° C. After addition. The mixture was stirred at this temperature for 30 min, and then NaBH(OAc)₃ (26.41 mg, 124.62 μmol, 2 eq) was added dropwise at 25° C. The resulting mixture was stirred at 50° C. for 16 hr. The reaction mixture was concentrated under vacuum to give a residue. The residue was purified by prep-HPLC (column: Waters Xbridge 150*25 mm*5 um;mobile phase:[water (ammonia hydroxide v/v)-ACN];gradient:45%-75% B over 10 min). followed by lyophilization. Compound 5-(1-(3,5-dichlorophenyl)-3-(4-isopropyl-2,2-dimethylpiperazine-1-carbonyl)-7-methoxy-4,5-dihydro-1H-benzo[g]indazol-8-yl)nicotinamide (0.99 mg, 1.45 mol, 2.33% yield, 95% purity) was obtained as a Off-white solid. LCMS (ESI): m/z[M+H] calcd for C34H37Cl2N6O3: 647.22; found: 647.2. 1H NMR (400 MHz, METHANOL-d4) δ=8.89 (d, J=2.0 Hz, 1H), 8.55 (d, J=2.1 Hz, 1H), 8.23 (t, J=2.1 Hz, 1H), 7.70-7.59 (m, 3H), 7.24 (s, 1H), 6.86 (s, 1H), 3.91 (s, 3H), 3.67 (br t, J=5.4 Hz, 2H), 3.16-3.08 (m, 2H), 2.83 (t, J=7.4 Hz, 2H), 2.72 (td, J=6.3, 13.1 Hz, 1H), 2.64 (br t, J=4.9 Hz, 2H), 2.47 (s, 2H), 1.59 (s, 6H), 1.07 (d, J=6.5 Hz, 6H). FIG. 100 shows the nuclear magnetic resonance of Compound 8-61.

### Synthesis of Compound 8-60

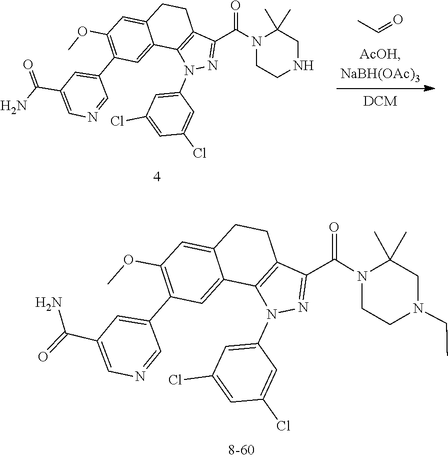

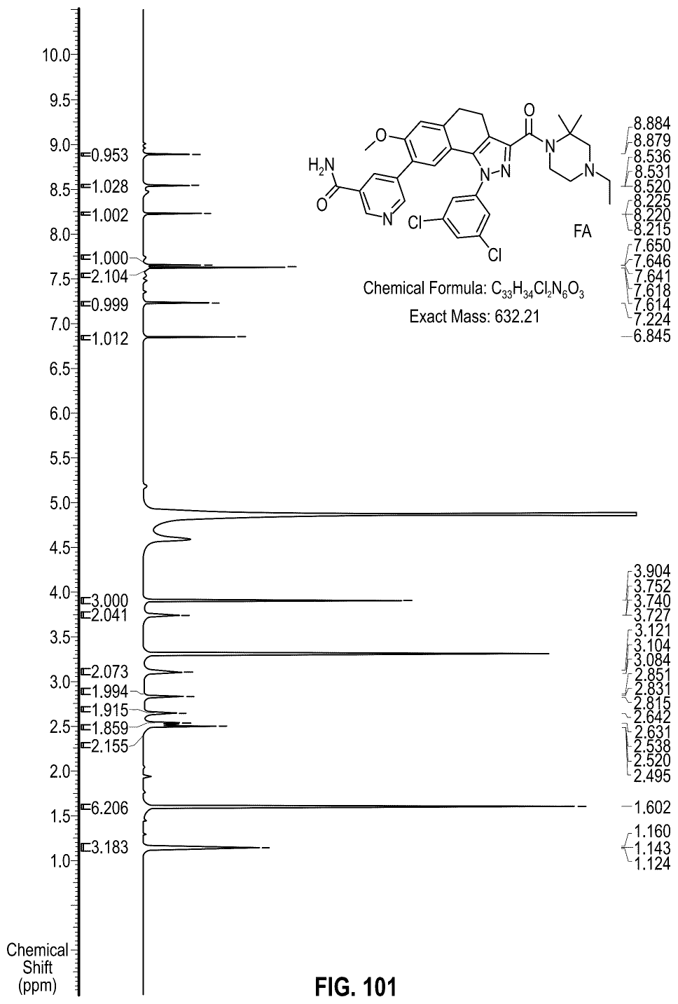

Compound 8-60 was synthesized via a method similar to example 8. LCMS (ESI): m/z[M+H] calcd for C₃₄H₃₇Cl₂N₆O₅: 633.21; found: 633.2. ¹H NMR (400 MHz, METHANOL-d4) δ=8.88 (d, J=2.0 Hz, 1H), 8.55-8.51 (m, 1H), 8.22 (t, J=2.1 Hz, 1H), 7.66-7.63 (m, 1H), 7.62 (d, J=1.8 Hz, 2H), 7.22 (s, 1H), 6.84 (s, 1H), 3.90 (s, 3H), 3.77-3.70 (m, 2H), 3.14-3.07 (m, 2H), 2.83 (t, J=7.3 Hz, 2H), 2.64 (br t, J=4.6 Hz, 2H), 2.53 (br d, J=7.3 Hz, 2H), 2.50 (s, 2H), 1.60 (s, 6H), 1.14 (t, J=7.2 Hz, 3H). FIG. 101 shows the nuclear magnetic resonance of Compound 8-60.

### Synthesis of Compound 8-63

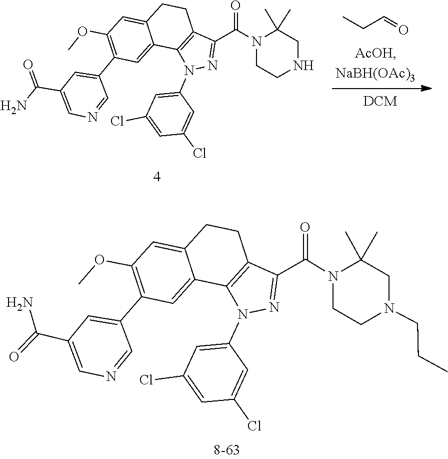

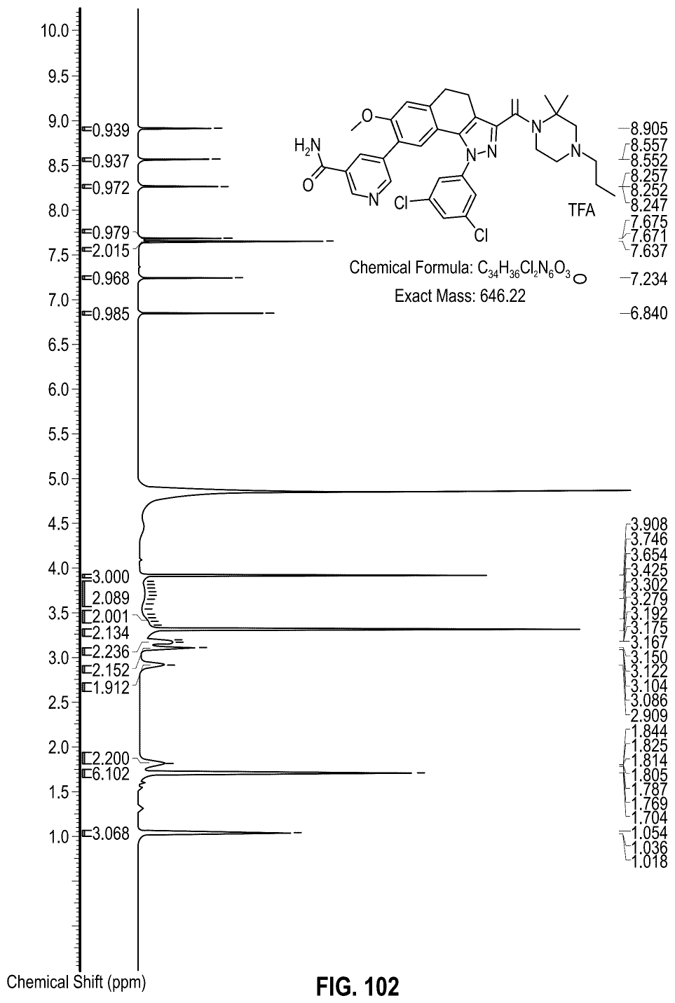

Compound 8-63 was synthesized via a method similar to example 8. LCMS (ESI): m/z[M+H] calcd for C₃₆H₃₇Cl₂N₆O₅F₃: 647.22; found: 647.2. ¹H NMR (400 MHz, METHANOL-d₄) δ=8.90 (d, J=2.0 Hz, 1H), 8.55 (d, J=2.0 Hz, 1H), 8.25 (t, J=2.0 Hz, 1H), 7.69-7.66 (m, 1H), 7.63 (d, J=1.8 Hz, 2H), 7.23 (s, 1H), 6.84 (s, 1H), 3.91 (s, 3H), 3.85-3.52 (m, 2H), 3.50-3.33 (m, 2H), 3.30-3.23 (m, 2H), 3.17 (br dd, J=6.8, 9.9 Hz, 2H), 3.13-3.07 (m, 2H), 2.91 (br d, J=1.6 Hz, 2H), 1.89-1.76 (m, 2H), 1.70 (s, 6H), 1.04 (t, J=7.4 Hz, 3H). FIG. 102 shows the nuclear magnetic resonance of Compound 8-63.

### Synthesis of Compound 8-58

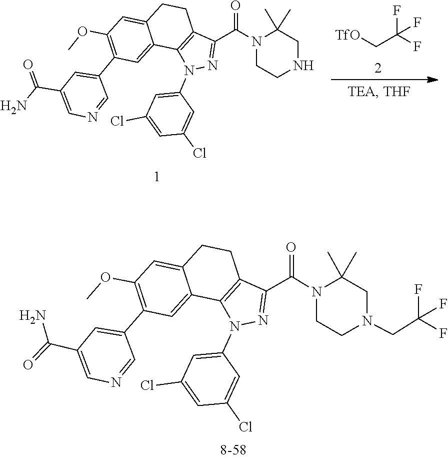 [SMILES: COc1cc2c(cc1-c1cncc(C(N)=O)c1)-c1c(c(C(=O)N3CCNCC3(C)C)nn1-c1cc(Cl)cc(Cl)c1)CC2 ; O=S(=O)(OCC(F)(F)F)C(F)(F)F ; COc1cc2c(cc1-c1cncc(C(N)=O)c1)-c1c(c(C(=O)N3CCN(CC(F)(F)F)CC3(C)C)nn1-c1cc(Cl)cc(Cl)c1)CC2]

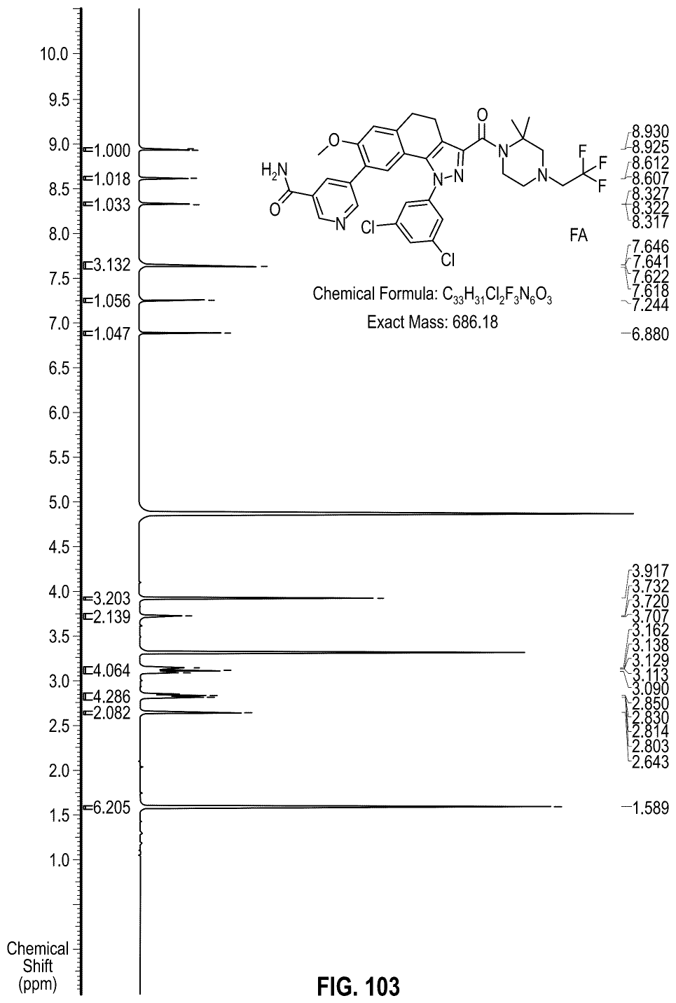

To a solution of 5-(1-(3,5-dichlorophenyl)-3-(2,2-dimethylpiperazine-1-carbonyl)-7-methoxy-4,5-dihydro-1H-benzo[g]indazol-8-yl)nicotinamide (80 mg, 132.12 μmol, 1 eq) and TEA (53.48 mg, 528.48 μmol, 73.56 μL, 4 eq) in THF (1 mL) was added 2,2,2-trifluoroethyl trifluoromethanesulfonate (30.67 mg, 132.12 μmol, 1 eq).The mixture was stirred at 40° C. for 5 hr. The pH of the reaction mixture was adjusted to 5 with 1 M HCl aqueous solution and then was diluted with H2O 20 mL and extracted with EtOAc 20 mL (10 mL*2). The combined organic layers were washed with brine 20 mL (10 mL*2), dried over Na2SO4, filtered and concentrated under reduced pressure to give a residue. The residue was purified by prep-HPLC (column: YMC-Actus Triart C18 150*30 mm*7 um;mobile phase:[water(FA)-ACN];gradient:60%-90% B over 10 min). followed by lyophilization. Compound 5-(1-(3,5-dichlorophenyl)-3-(2,2-dimethyl-4-(2,2,2-trifluoroethyl)piperazine-1-carbonyl)-7-methoxy-4,5-dihydro-1H-benzo[g]indazol-8-yl)nicotinamide (27.2 mg, 35.60 μmol, 26.94% yield, 96% purity, FA) was obtained as a white solid. LCMS (ESI): m/z[M+H] calcd for C34H34Cl2N6O5F3: 687.18; found: 687.1. 1H NMR (400 MHz, METHANOL-d4) δ=8.93 (d, J=1.8 Hz, 1H), 8.61 (d, J=2.0 Hz, 1H), 8.32 (t, J=1.9 Hz, 1H), 7.66-7.60 (m, 3H), 7.24 (s, 1H), 6.88 (s, 1H), 3.92 (s, 3H), 3.72 (t, J=5.1 Hz, 2H), 3.15-3.07 (m, 4H), 2.87-2.78 (m, 4H), 2.64 (s, 2H), 1.59 (s, 6H). FIG. 103 shows the nuclear magnetic resonance of Compound 8-58.

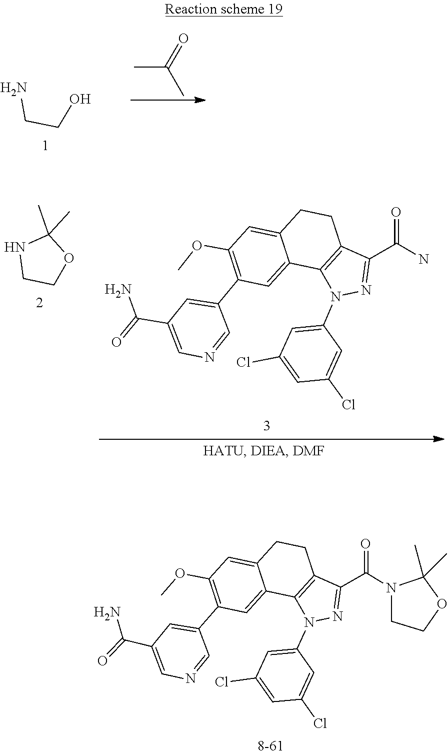

### Synthesis of 2,2-dimethyloxazolidine

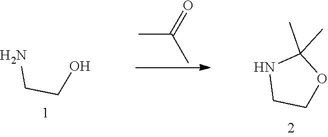

A mixture of 2-aminoethan-1-ol (50 mg, 818.56 μmol, 49.41 μL, 1 eq) in propan-2-one (395.00 mg, 6.80 mmol, 0.5 mL, 8.31 eq) was stirred at 60° C. for 16 hr. The reaction mixture was concentrated in vacuo. Product 2,2-dimethyloxazolidine (80 mg, 522.02 μmol, 63.77% yield, 66% purity) (a mixture of SM and DP with a ratio of 2 to 1) was obtained as a yellow oil. LCMS (ESI): m/z[M+H] calcd for C5H13NO: 102.08; found: 102.1.

### Synthesis of Compound 8-51

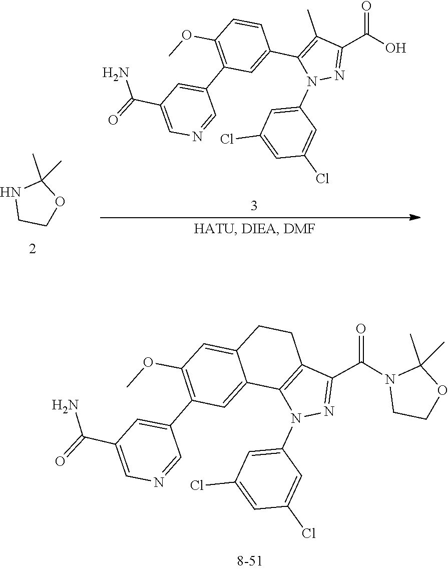

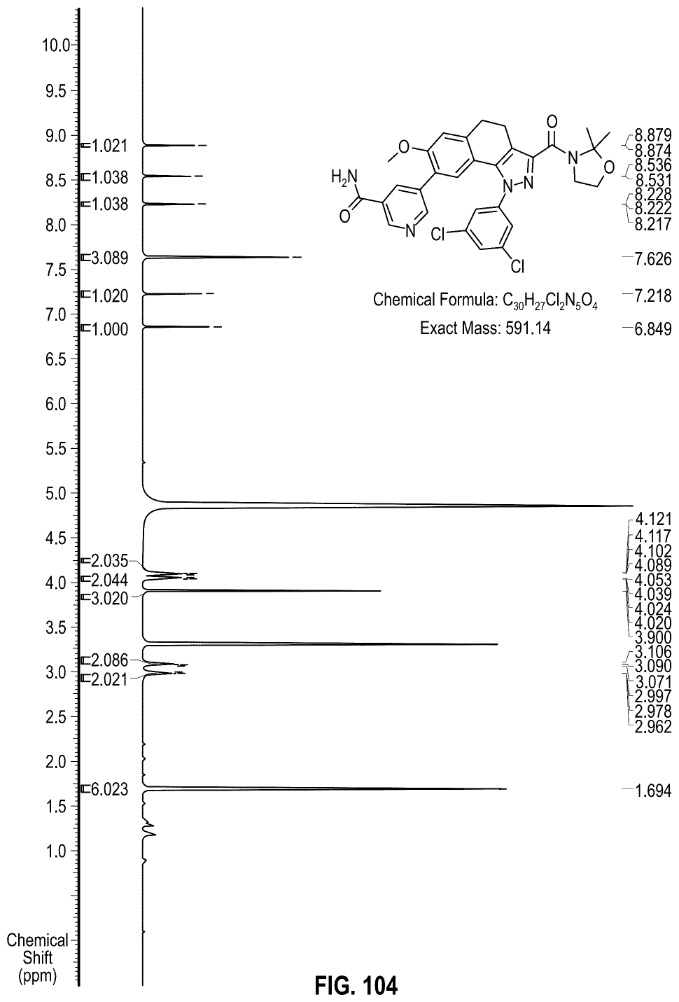

To a solution of 8-(5-carbamoylpyridin-3-yl)-1-(3,5-dichlorophenyl)-7-methoxy-4,5-dihydro-1H-benzo[g]indazole-3-carboxylic acid (80 mg, 157.07 μmol, 1 eq) in DMF (1 mL) was added dropwise HATU (71.67 mg, 188.48 μmol, 1.2 eq) and DIEA (60.90 mg, 471.20 μmol, 82.07 μL, 3 eq) After addition. The mixture was stirred at this temperature for 30 min, and then 2,2-dimethyloxazolidine (30 mg, 195.76 μmol, 1.25 eq) was added dropwise. The resulting mixture was stirred at 25° C. for 16 hr. The reaction mixture was diluted with EtOAc (10 mL) and then filtered through a celite pad. The filtrate was concentrated. the filtrate was purified by prep-HPLC (column: Waters Xbridge 150*25 mm*5 um;mobile phase:[water(NH4HCO3)-ACN];gradient:322%-62% B over 15 min). Compound 5-(1-(3,5-dichlorophenyl)-3-(2,2-dimethyloxazolidine-3-carbonyl)-7-methoxy-4,5-dihydro-1H-benzo[g]indazol-8-yl)nicotinamide (15.5 mg, 25.64 μmol, 16.32% yield, 98% purity) was obtained as a white solid. LCMS (ESI): m/z[M+H] calcd for C30H28Cl2N5O4: 592.14; found: 592.1. 1H NMR (400 MHz, METHANOL-d4) δ=8.88 (d, J=2.0 Hz, 1H), 8.53 (d, J=2.1 Hz, 1H), 8.22 (t, J=2.1 Hz, 1H), 7.63 (s, 3H), 7.22 (s, 1H), 6.85 (s, 1H), 4.13-4.08 (m, 2H), 4.07-4.00 (m, 2H), 3.90 (s, 3H), 3.12-3.05 (m, 2H), 3.01-2.94 (m, 2H), 1.69 (s, 6H). FIG. 104 shows the nuclear magnetic resonance of Compound 8-51.

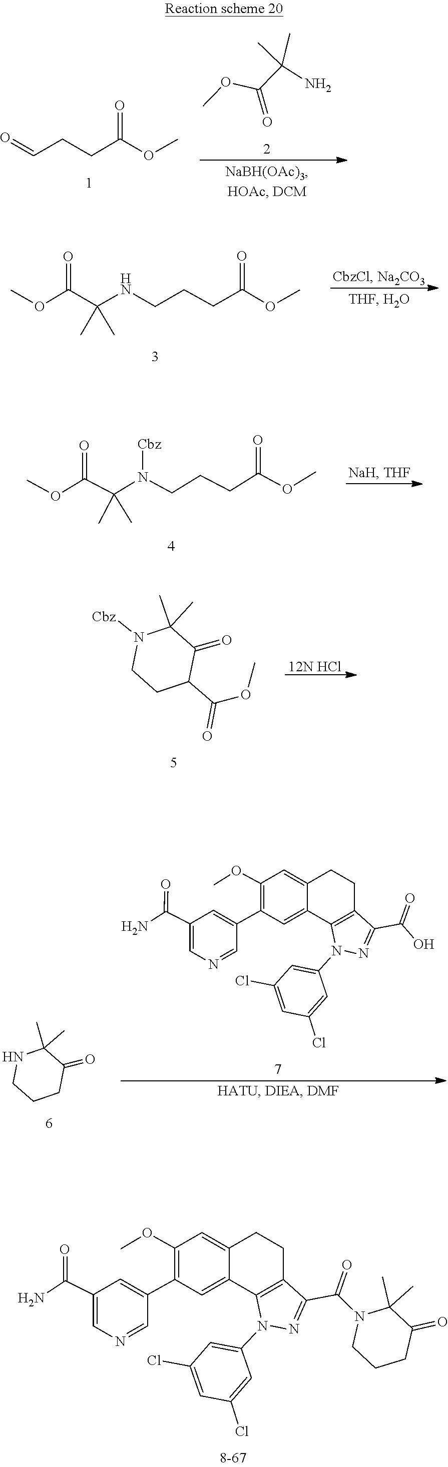

### Synthesis of methyl 4-((1-methoxy-2-methyl-1-oxopropan-2-yl)amino)butanoate

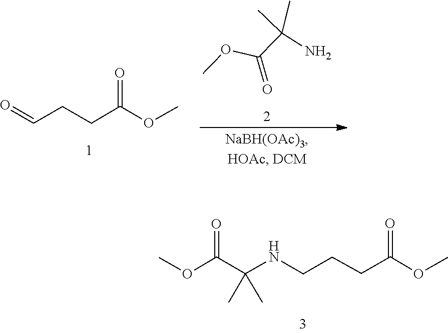

A mixture of methyl 4-oxobutanoate (5 g, 43.06 mmol, 1 eq), TEA (8.71 g, 86.12 mmol, 11.99 mL, 2 eq), NaBH(OAc)₃ (22.82 g, 107.65 mmol, 2.5 eq) and methyl 2-amino-2-methylpropanoate (6.05 g, 51.67 mmol, 1.2 eq) in DCM (50 mL), and then the mixture was stirred at 25° C. for 16 hr under N2 atmosphere. The reaction mixture was quenched by addition saturated NaHCO₃ aqueous solution (50 mL), and then diluted with H2O 100 mL and extracted with EtOAc 200 mL (100 mL*2). The combined organic layers were washed with brine 200 mL (100 mL*2), dried over Na₂SO4, filtered and concentrated under reduced pressure to give a residue. Compound methyl 4-((1-methoxy-2-methyl-1-oxopropan-2-yl)amino)butanoate (8.7 g, 40.04 mmol, 92.99% yield) was obtained as a yellow oil confirmed. 1H NMR (400 MHz, DMSO-d6) δ=4.26 (t, J=7.1 Hz, 1H), 3.58 (s, 6H), 2.51-2.48 (m, 6H), 1.24-1.20 (m, 6H).

### Synthesis of methyl 4-(((benzyloxy)carbonyl)(1-methoxy-2-methyl-1-oxopropan-2-yl)amino)butanoate

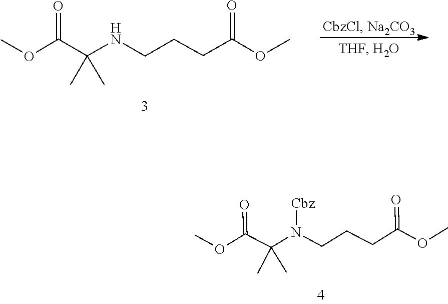

To a solution of methyl 4-((1-methoxy-2-methyl-1-oxopropan-2-yl)amino)butanoate (7.7 g, 35.44 mmol, 1 eq) in THF (80 mL) and H2O (80 mL) was added Na2CO3 (9.39 g, 88.60 mmol, 2.5 eq) and CbzCl (12.09 g, 70.88 mmol, 10.12 mL, 2 eq).The mixture was stirred at 25° C. for 16 hr. The reaction mixture was diluted with H2O 200 mL and extracted with EtOAc 400 mL (200 mL*2). The combined organic layers were washed with brine 200 mL (100 mL*2), dried over Na2SO4, filtered and concentrated under reduced pressure to give a residue. The residue was purified by prep-HPLC (FA condition). Compound methyl 4-(((benzyloxy)carbonyl)(1-methoxy-2-methyl-1-oxopropan-2-yl)amino)butanoate (6.5 g, 18.50 mmol, 52.19% yield) was obtained as a yellow oil. LCMS (ESI): m/z[M+Na] calcd for C18H25O6NNa: 374.16; found: 374.1. 1H NMR (400 MHz, DMSO-d6) δ=7.40-7.34 (m, 2H), 7.34-7.29 (m, 3H), 5.05 (s, 2H), 3.56 (s, 3H), 3.48 (br s, 2H), 3.31 (br s, 3H), 2.31 (t, J=7.3 Hz, 2H), 1.76 (quin, J=7.4 Hz, 2H), 1.40 (s, 6H).

### Synthesis of 1-benzyl 4-methyl 2,2-dimethyl-3-oxopiperidine-1,4-dicarboxylate

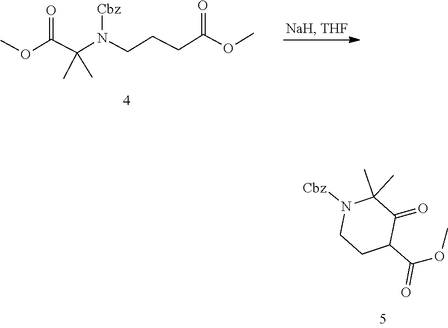

To a solution of methyl 4-(((benzyloxy)carbonyl)(1-methoxy-2-methyl-1-oxopropan-2-yl)amino)butanoate (6.5 g, 18.50 mmol, 1 eq) in THF (70 mL) was added NaH (1.48 g, 37.00 mmol, 60% purity, 2 eq). The mixture was stirred at 40° C. for 4 hr. The reaction mixture was quenched by addition saturated NH4Cl aqueous solution (50 mL) and then was acidified to pH=6 with 1 M HCl aqueous solution, and then diluted with H2O 100 mL and extracted with EtOAc 200 mL (100 mL*2). The combined organic layers were washed with brine 200 mL (100 mL*2), dried over Na2SO4, filtered and concentrated under reduced pressure to give a residue. The residue was purified by flash silica gel chromatography (ISCO®; 80 g SepaFlash® Silica Flash Column, Eluent of 15% Ethylacetate/Petroleum ethergradient @100 mL/min) and then was purified by prep-HPLC (basic condition). Compound 1-benzyl 4-methyl 2,2-dimethyl-3-oxopiperidine-1,4-dicarboxylate (2.1 g, 6.58 mmol, 35.55% yield) was obtained as a yellow oil. 1H NMR (400 MHz, DMSO-d6) δ=7.42-7.29 (m, 5H), 5.08 (s, 2H), 3.74 (s, 3H), 3.51 (t, J=5.4 Hz, 2H), 2.27 (t, J=5.4 Hz, 2H), 1.59 (s, 6H).

### Synthesis of 2,2-dimethylpiperidin-3-one

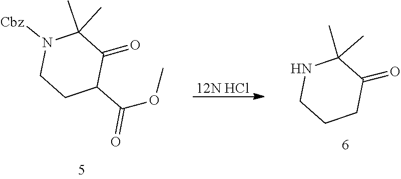

A mixture of 1-benzyl 4-methyl 2,2-dimethyl-3-oxopiperidine-1,4-dicarboxylate (700 mg, 2.19 mmol, 1 eq) and HCl (12 M, 7.00 mL, 38.32 eq) in H2O (10 mL) was stirred at 100° C. for 48 hr. The reaction mixture was concentrated under reduced pressure to give a residue. Compound 2,2-dimethylpiperidin-3-one (150 mg, 1.18 mmol, 53.81% yield) was obtained as a yellow oil. 1H NMR (400 MHz, CHLOROFORM-d) δ=3.46-3.36 (m, 2H), 2.67 (br t, J=6.7 Hz, 2H), 2.34 (br s, 2H), 1.67 (s, 6H).

### Synthesis of Compound 8-67

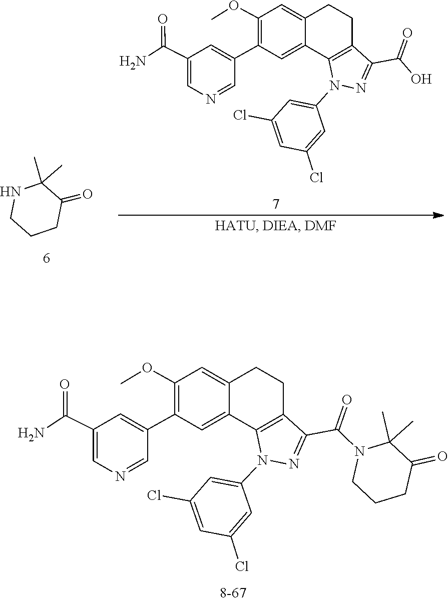

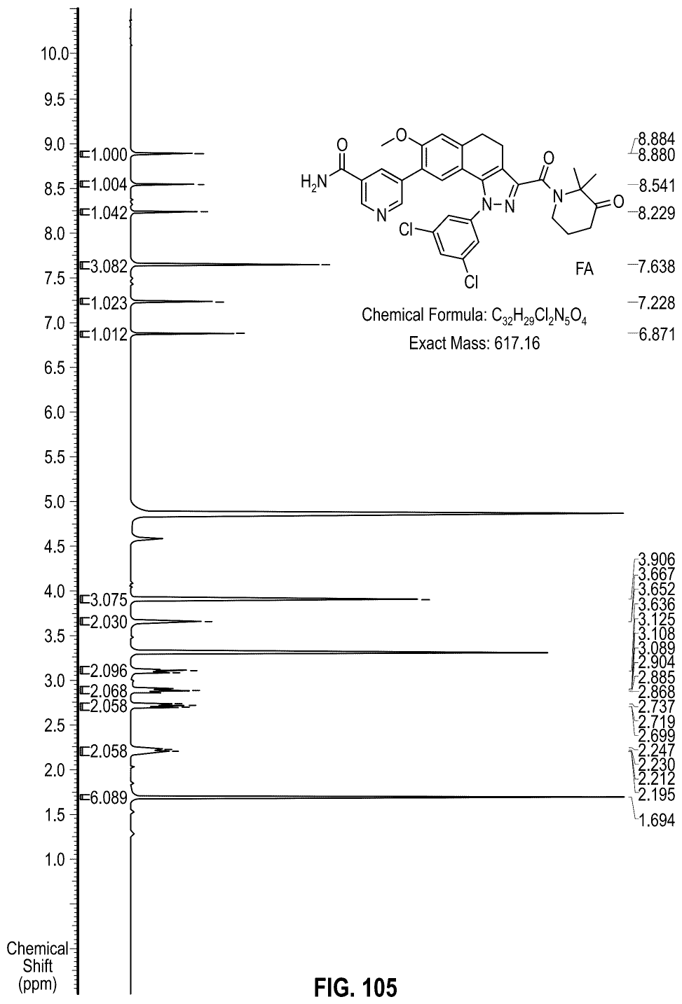

To a solution of 8-(5-carbamoylpyridin-3-yl)-1-(3,5-dichlorophenyl)-7-methoxy-4,5-dihydro-1H-benzo[g]indazole-3-carboxylic acid (40 mg, 78.53 μmol, 1 eq) in DMF (1 mL) was added dropwise HATU (44.79 mg, 117.80 μmol, 1.5 eq) and DIEA (30.45 mg, 235.60 μmol, 41.04 μL, 3 eq). After addition. The mixture was stirred at this temperature for 0.5 hr, and then 2,2-dimethylpiperidin-3-one (29.96 mg, 235.60 μmol, 3 eq) was added. The resulting mixture was stirred at 25° C. for 4 hr. The reaction mixture was diluted with EtOAc (10 mL) and then filtered through a celite pad. The filtrate was concentrated. The residue was purified by prep-HPLC (column: YMC-Actus Triart C18 150*30 mm*7 um;mobile phase:[water(FA)-ACN];gradient:50%-80% B over 10 min).followed by lyophilization. Compound 5-(1-(3,5-dichlorophenyl)-3-(2,2-dimethyl-3-oxopiperidine-1-carbonyl)-7-methoxy-4,5-dihydro-1H-benzo[g]indazol-8-yl)nicotinamide (14.05 mg, 20.51 μmol, 26.11% yield, 97% purity, FA) was obtained as a green solid. LCMS (ESI): m/z[M+H] calcd for C33H32Cl2N5O6: 618.16; found: 618.2. 1H NMR (400 MHz, METHANOL-d4) δ=8.88 (d, J=1.6 Hz, 1H), 8.54 (d, J=1.7 Hz, 1H), 8.23 (s, 1H), 7.64 (s, 3H), 7.23 (s, 1H), 6.87 (s, 1H), 3.91 (s, 3H), 3.65 (t, J=6.1 Hz, 2H), 3.15-3.05 (m, 2H), 2.93-2.84 (m, 2H), 2.75-2.67 (m, 2H), 2.21 (quin, J=6.8 Hz, 2H), 1.69 (s, 6H). FIG. 105 shows the nuclear magnetic resonance of Compound 8-67.

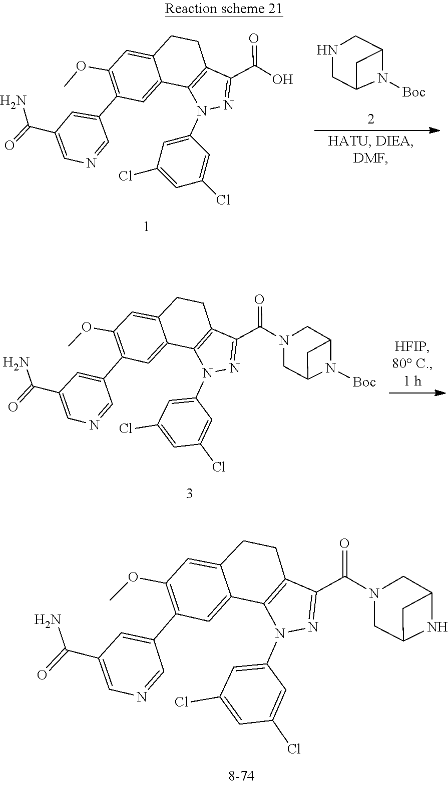 [SMILES: COc1cc2c(cc1-c1cncc(C(N)=O)c1)-c1c(c(C(=O)O)nn1-c1cc(Cl)cc(Cl)c1)CC2 ; CC(C)(C)OC(=O)N1C2CNCC1C2 ; COc1cc2c(cc1-c1cncc(C(N)=O)c1)-c1c(c(C(=O)N3CC4CC(C3)N4C(=O)OC(C)(C)C)nn1-c1cc(Cl)cc(Cl)c1)CC2 ; COc1cc2c(cc1-c1cncc(C(N)=O)c1)-c1c(c(C(=O)N3CC4CC(C3)N4)nn1-c1cc(Cl)cc(Cl)c1)CC2]

### Synthesis of tert-butyl 3-(8-(5-carbamoylpyridin-3-yl)-1-(3,5-dichlorophenyl)-7-methoxy-4,5-dihydro-1H-benzo[g]indazole-3-carbonyl)-3,6-diazabicyclo[3.1.1]heptane-6-carboxylate

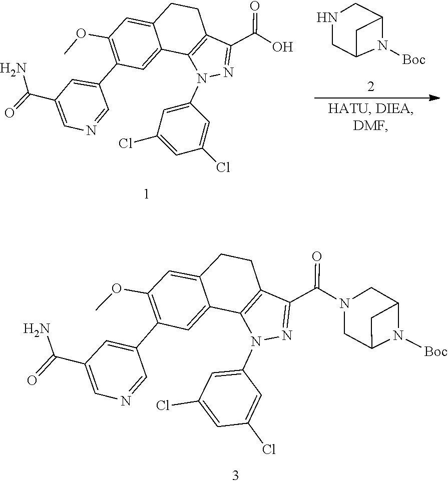 [SMILES: COc1cc2c(cc1-c1cncc(C(N)=O)c1)-c1c(c(C(=O)O)nn1-c1cc(Cl)cc(Cl)c1)CC2 ; CC(C)(C)OC(=O)N1C2CNCC1C2 ; COc1cc2c(cc1-c1cncc(C(N)=O)c1)-c1c(c(C(=O)N3CC4CC(C3)N4C(=O)OC(C)(C)C)nn1-c1cc(Cl)cc(Cl)c1)CC2]

To a solution of 8-(5-carbamoylpyridin-3-yl)-1-(3,5-dichlorophenyl)-7-methoxy-4,5-dihydro-1H-benzo[g]indazole-3-carboxylic acid (80 mg, 157.07 μmol, 1 eq) in DMF (1 mL) was added HATU (89.58 mg, 235.60 μmol, 1.5 eq) and DIEA (60.90 mg, 471.20 μmol, 82.07 μL, 3 eq).The tert-butyl 3,6-diazabicyclo[3.1.1]heptane-6-carboxylate (34.25 mg, 172.77 μmol, 1.1 eq) was added after 15 min.The mixture was stirred at 25° C. for 1.75 hr. The pH of the reaction mixture was adjusted to 7 with 1 M HCl. The mixture was poured into water(10 mL) and extracted with ethyl acetate(3*20 mL). The organic phase was separated, washed with Saturated sodium chloride solution (10 mL), dried over Na2SO4, filtered and concentrated under reduced pressure to give a residue. The crude product tert-butyl 3-(8-(5-carbamoylpyridin-3-yl)-1-(3,5-dichlorophenyl)-7-methoxy-4,5-dihydro-1H-benzo[g]indazole-3-carbonyl)-3,6-diazabicyclo[3.1.1]heptane-6-carboxylate (120 mg, crude) as a black brown oil which was used into the next step without further purification. LCMS (ESI): m/z[M+H] calcd for C35H34N6O5Cl2: 689.20; found: 689.20.

### Synthesis of Compound 8-74

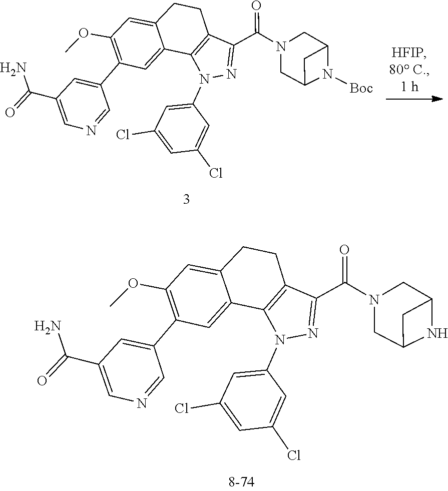 [SMILES: COc1cc2c(cc1-c1cncc(C(N)=O)c1)-c1c(c(C(=O)N3CC4CC(C3)N4C(=O)OC(C)(C)C)nn1-c1cc(Cl)cc(Cl)c1)CC2 ; COc1cc2c(cc1-c1cncc(C(N)=O)c1)-c1c(c(C(=O)N3CC4CC(C3)N4)nn1-c1cc(Cl)cc(Cl)c1)CC2]

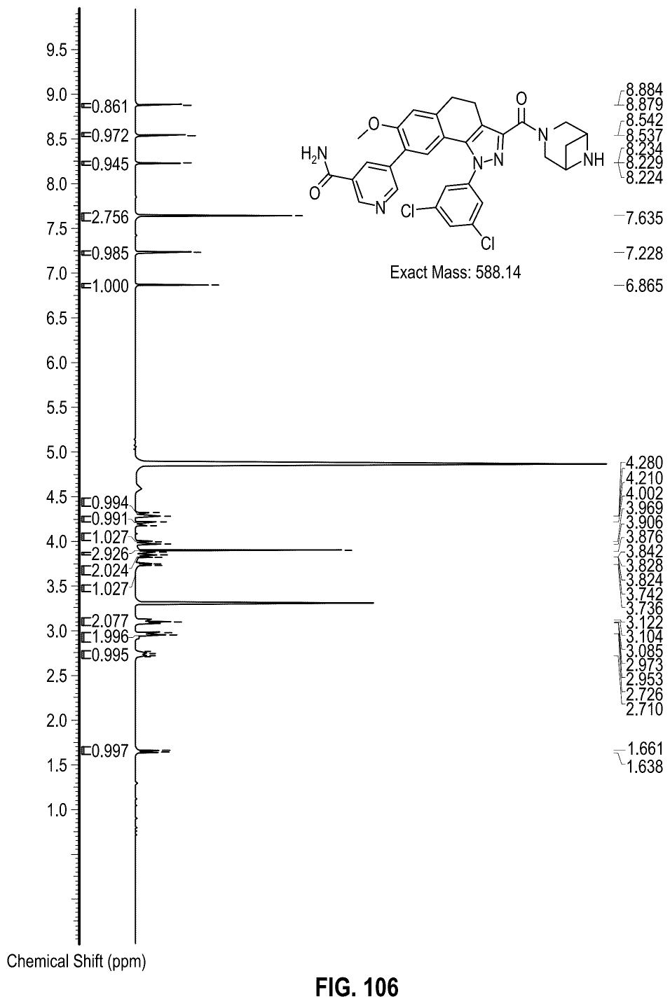

A solution of tert-butyl 3-(8-(5-carbamoylpyridin-3-yl)-1-(3,5-dichlorophenyl)-7-methoxy-4,5-dihydro-1H-benzo[g]indazole-3-carbonyl)-3,6-diazabicyclo[3.1.1]heptane-6-carboxylate (90 mg, 130.51 μmol, 1 eq) in 1,1,1,3,3,3-hexafluoropropan-2-ol (1 mL) was stirred at 80° C. for 1 hr. The mixture was concentrated to give a residue. The residue was purified by prep-HPLC (TFA condition;column: Waters Xbridge 150*25 mm*5 um;mobile phase: [water(NH4HCO3)-ACN];gradient:25%-55% B over 9 min). Compound 5-(3-(3,6-diazabicyclo[3.1.1]heptane-3-carbonyl)-1-(3,5-dichlorophenyl)-7-methoxy-4,5-dihydro-1H-benzo[g]indazol-8-yl)nicotinamide (49.41 mg, 82.14 μmol, 62.94% yield, 98% purity) was obtained as a white solid. LCMS (ESI): m/z[M+H] calcd for C30H26N6O3 Cl2: 589.14; found: 589.2. 1H NMR (400 MHz, METHANOL-d4) δ=8.88 (d, J=2.0 Hz, 1H), 8.54 (d, J=2.0 Hz, 1H), 8.25-8.20 (m, 1H), 7.64 (s, 3H), 7.23 (s, 1H), 6.86 (s, 1H), 4.34-4.25 (m, 1H), 4.24-4.16 (m, 1H), 4.02-3.95 (m, 1H), 3.91 (s, 3H), 3.89-3.79 (m, 2H), 3.74 (d, J=2.4 Hz, 1H), 3.15-3.05 (m, 2H), 3.00-2.90 (m, 2H), 2.78-2.69 (m, 1H), 1.65 (d, J=9.2 Hz, 1H). FIG. 106 shows the nuclear magnetic resonance of Compound 8-74.

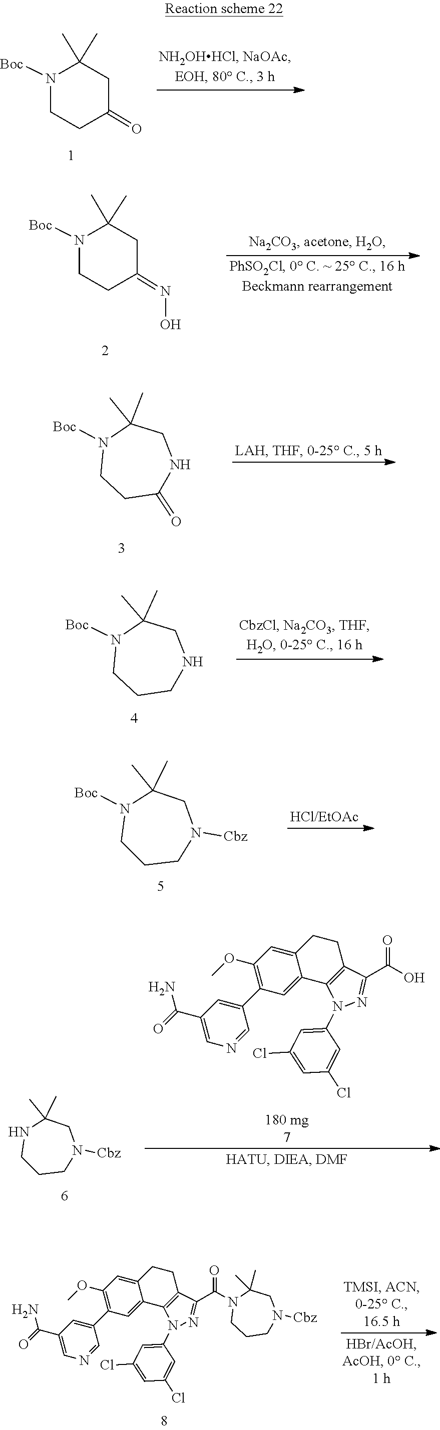

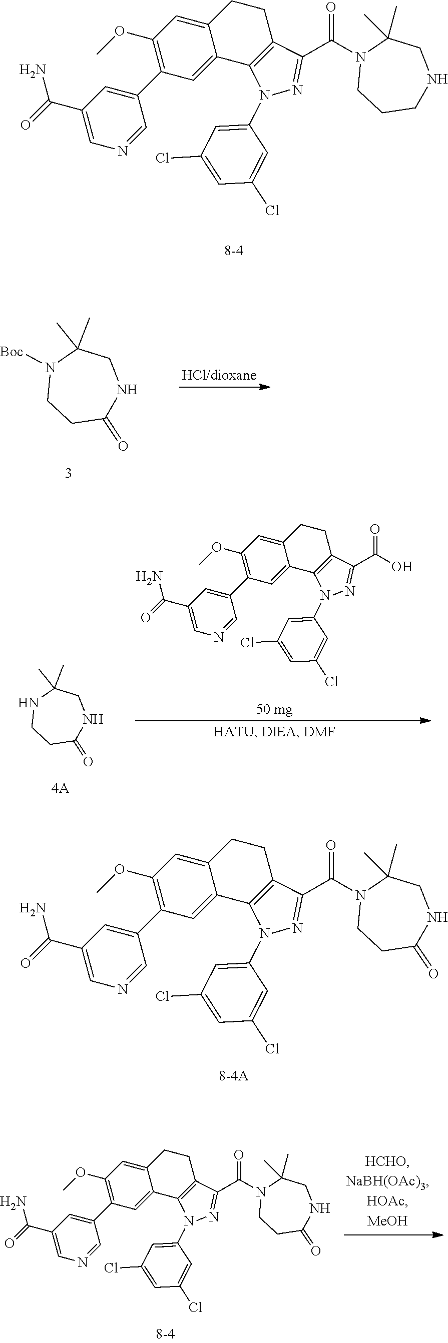

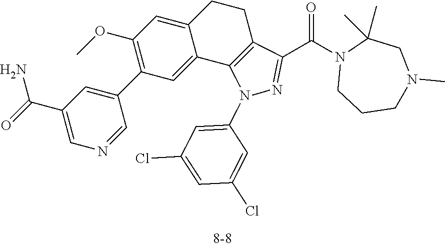 [SMILES: COc1cc2c(cc1-c1cncc(C(N)=O)c1)-c1c(c(C(=O)N3CCCN(C)CC3(C)C)nn1-c1cc(Cl)cc(Cl)c1)CC2]

### Synthesis of tert-butyl (4E)-4-hydroxyimino-2,2-dimethyl-piperidine-1-carboxylate

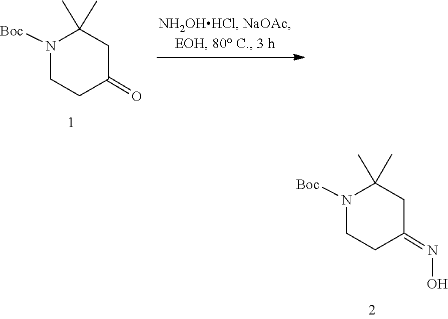 [SMILES: CC(C)(C)OC(=O)N1CCC(=O)CC1(C)C ; CC(C)(C)OC(=O)N1CC/C(=N\O)CC1(C)C]

To a solution of tert-butyl 2,2-dimethyl-4-oxo-piperidine-1-carboxylate (1 g, 4.40 mmol, 1 eq) in EtOH (10 mL) was added NaOAc (1.80 g, 22.00 mmol, 5 eq) and hydroxylamine; hydrochloride (1.53 g, 22.00 mmol, 5 eq). The mixture was stirred at 80° C. for 3 h. The reaction mixture was diluted with ice water (50 mL). The aqueous layer was extracted with ethyl acetate (50 mL*3). The combined organic layers were dried with anhydrous Na2SO4, filtered and concentrated in vacuum to give a residue. The crude product tert-butyl(4E)-4-hydroxyimino-2,2-dimethyl-piperidine-1-carboxylate (1 g, crude) as a white solid was used into the next step without further purification. 1H NMR (400 MHz, CHLOROFORM-d) δ=3.69 (t, J=6.1 Hz, 2H), 2.61 (t, J=6.1 Hz, 2H), 2.44 (s, 2H), 1.48 (s, 9H), 1.42 (s, 6H).

### Synthesis of tert-butyl 2,2-dimethyl-5-oxo-1,4-diazepane-1-carboxylate

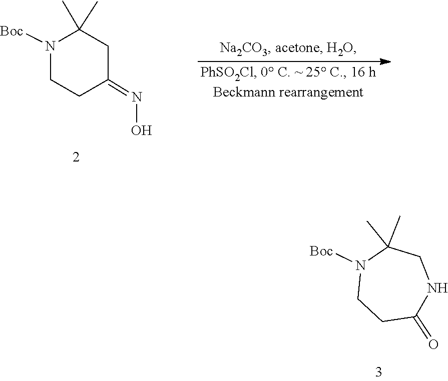 [SMILES: CC(C)(C)OC(=O)N1CC/C(=N\O)CC1(C)C ; CC(C)(C)OC(=O)N1CCC(=O)NCC1(C)C]

To a solution of tert-butyl (4E)-4-hydroxyimino-2,2-dimethyl-piperidine-1-carboxylate (900 mg, 3.71 mmol, 1 eq) and Na2CO₃ (1.57 g, 14.86 mmol, 4 eq) in acetone(20 mL) and H₂O(20 mL) was added benzenesulfonyl chloride (1.31 g, 7.43 mmol, 947.98 μL, 2 eq) dropwise at 0° C. The mixture was stirred at 25° C. for 16 hr. The reaction mixture was diluted with ice water (50 mL). The aqueous layer was extracted with ethyl acetate (50 mL*3). The combined organic layers were dried with anhydrous Na2SO4, filtered and concentrated in vacuum to give a residue. The crude product tert-butyl 2,2-dimethyl-5-oxo-1, 4-diazepane-1-carboxylate (850 mg, crude) as a white solid was used into the next step without further purification. 1H NMR (400 MHz, CHLOROFORM-d) δ=6.26 (br s, 1H), 3.71 (t, J=6.3 Hz, 2H), 3.28 (br d, J=6.3 Hz, 2H), 2.71 (t, J=6.3 Hz, 2H), 1.47 (s, 9H), 1.45 (s, 6H).

### Synthesis of tert-butyl 2,2-dimethyl-1,4-diazepane-1-carboxylate

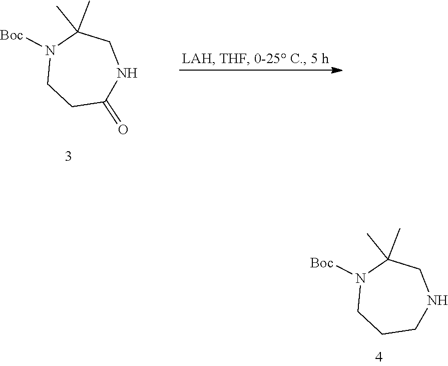 [SMILES: CC(C)(C)OC(=O)N1CCC(=O)NCC1(C)C ; CC(C)(C)OC(=O)N1CCCNCC1(C)C]

To a solution of tert-butyl 2,2-dimethyl-5-oxo-1,4-diazepane-1-carboxylate (200 mg, 825.38 μmol, 1 eq) in THF (5 mL) was added LiAlH4 (46.99 mg, 1.24 mmol, 1.5 eq) at 0° C. The mixture was stirred at 25° C. for 5 hr. The residue was quenched with Na2SO₄·10H2O (1 g) and then filtered. The filtrate was concentrated. The crude product tert-butyl 2,2-dimethyl-1, 4-diazepane-1-carboxylate (180 mg, 788.33 μmol, 95.51% yield) as a colorless oil was used into the next step without further purification. 1H NMR (400 MHz, CHLOROFORM-d) δ=3.56-3.49 (m, 2H), 2.87-2.83 (m, 2H), 2.81 (s, 2H), 1.48-1.46 (m, 11H), 1.38 (s, 6H).

### Synthesis of 04-benzyl 01-tert-butyl 2,2-dimethyl-1,4-diazepane-1,4-dicarboxylate

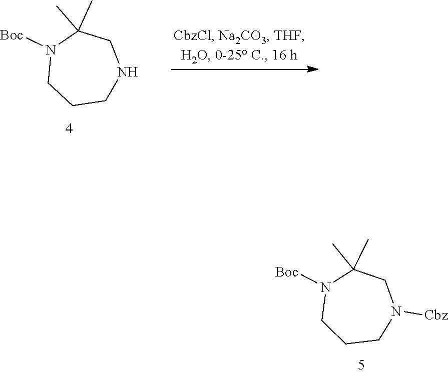 [SMILES: CC(C)(C)OC(=O)N1CCCNCC1(C)C ; CC(C)(C)OC(=O)N1CCCN(C(=O)OCc2ccccc2)CC1(C)C]

To a solution of tert-butyl 2,2-dimethyl-1,4-diazepane-1-carboxylate (180 mg, 788.33 μmol, 1 eq) and Na2CO3 (250.66 mg, 2.36 mmol, 3 eq) in THF (4 mL) and H2O (2 mL) was added benzyl carbonochloridate (268.97 mg, 1.58 mmol, 225.08 μL, 2 eq). The mixture was stirred at 25° C. for 16 hr. The reaction mixture was diluted with ice water (20 mL). The aqueous layer was extracted with ethyl acetate (10 mL*3). The combined organic layers were dried with anhydrous Na2SO4, filtered and concentrated in vacuum to give a residue. The residue was purified by prep-HPLC (column: Waters Xbridge Prep OBD C18 150*40 mm*10 um; mobile phase:[water (NH4HCO3)-ACN]; gradient: 50%-80% B over 20 min), followed by lyophilization. Compound 04-benzyl 01-tert-butyl 2,2-dimethyl-1,4-diazepane-1,4-dicarboxylate (160 mg, 441.43 μmol, 55.99% yield) was obtained as a yellow oil. 1H NMR (400 MHz, CHLOROFORM-d) δ=7.40-7.31 (m, 5H), 5.14 (s, 2H), 3.67-3.41 (m, 6H), 1.78 (br s, 2H), 1.47 (s, 9H), 1.44-1.35 (m, 6H)

### Synthesis of benzyl 3,3-dimethyl-1,4-diazepane-1-carboxylate

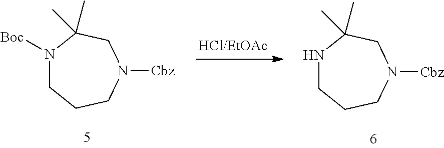 [SMILES: CC(C)(C)OC(=O)N1CCCN(C(=O)OCc2ccccc2)CC1(C)C ; CC1(C)CN(C(=O)OCc2ccccc2)CCCN1]

A solution of 04-benzyl 0i-tert-butyl 2,2-dimethyl-1,4-diazepane-1,4-dicarboxylate (160 mg, 441.43 μmol, 1 eq) in HCl/EtOAc (2 M, 2 mL, 9.06 eq) was stirred at 25° C. for 1 hr. The reaction mixture was concentrated under reduced pressure to give a residue. The crude product benzyl 3,3-dimethyl-1,4-diazepane-1-carboxylate (130 mg, crude, HCl) as a colorless oil was used into the next step without further purification.

### Synthesis of Benzyl4-[8-(5-carbamoyl-3-pyridyl)-1-(3,5-dichlorophenyl)-7-methoxy-4,5-dihydrobenzo[g]indazole-3-carbonyl]-3,3-dimethyl-1,4-diazepane-1-carboxylate

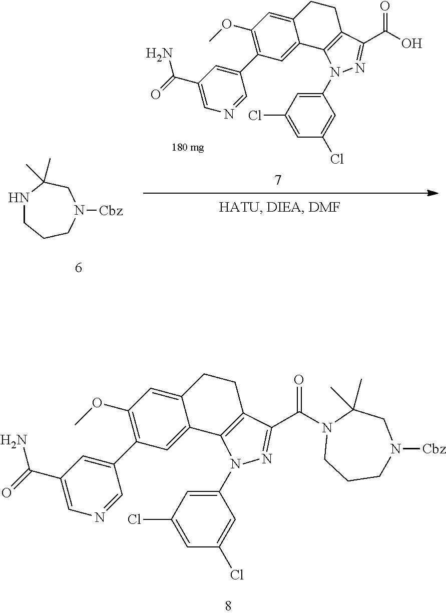 [SMILES: CC1(C)CN(C(=O)OCc2ccccc2)CCCN1 ; COc1cc2c(cc1-c1cncc(C(N)=O)c1)-c1c(c(C(=O)O)nn1-c1cc(Cl)cc(Cl)c1)CC2 ; COc1cc2c(cc1-c1cncc(C(N)=O)c1)-c1c(c(C(=O)N3CCCN(C(=O)OCc4ccccc4)CC3(C)C)nn1-c1cc(Cl)cc(Cl)c1)CC2]

To a solution of 8-(5-carbamoyl-3-pyridyl)-1-(3,5-dichlorophenyl)-7-methoxy-4,5-dihydrobenzo[g]indazole-3-carboxylic acid (180 mg, 353.40 μmol, 1 eq) in DMF (3 mL) was added HATU (161.25 mg, 424.08 μmol, 1.2 eq) and DIEA (137.02 mg, 1.06 mmol, 184.66 μL, 3 eq). The mixture stirred at 25° C. for 30 min. Then to the mixture was added a mixture of benzyl 3,3-dimethyl-1,4-diazepane-1-carboxylate (126.72 mg, 424.08 μmol, 1.2 eq, HCl) in DMF (1 mL). The mixture stirred at 25° C. for 16 hr. The mixture was diluted with ice water (10 mL). And then filtered to give the filter cake. The filter cake was collected and dried in vacuum to give a product. The crude product benzyl 4-[8-(5-carbamoyl-3-pyridyl)-1-(3,5-dichlorophenyl)-7-methoxy-4,5-dihydrobenzo[g]indazole-3-carbonyl]-3,3-dimethyl-1,4-diazepane-1-carboxylate (190 mg, 252.10 μmol, 71.34% yield) as a yellow solid was used into the next step without further purification.

### Synthesis of Compound 8-4

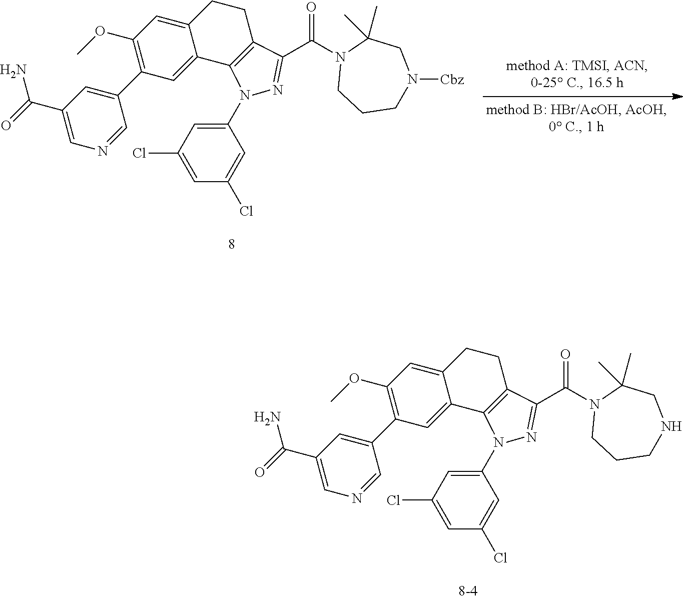 [SMILES: COc1cc2c(cc1-c1cncc(C(N)=O)c1)-c1c(c(C(=O)N3CCCN(C(=O)OCc4ccccc4)CC3(C)C)nn1-c1cc(Cl)cc(Cl)c1)CC2 ; COc1cc2c(cc1-c1cncc(C(N)=O)c1)-c1c(c(C(=O)N3CCCNCC3(C)C)nn1-c1cc(Cl)cc(Cl)c1)CC2]

### Method A

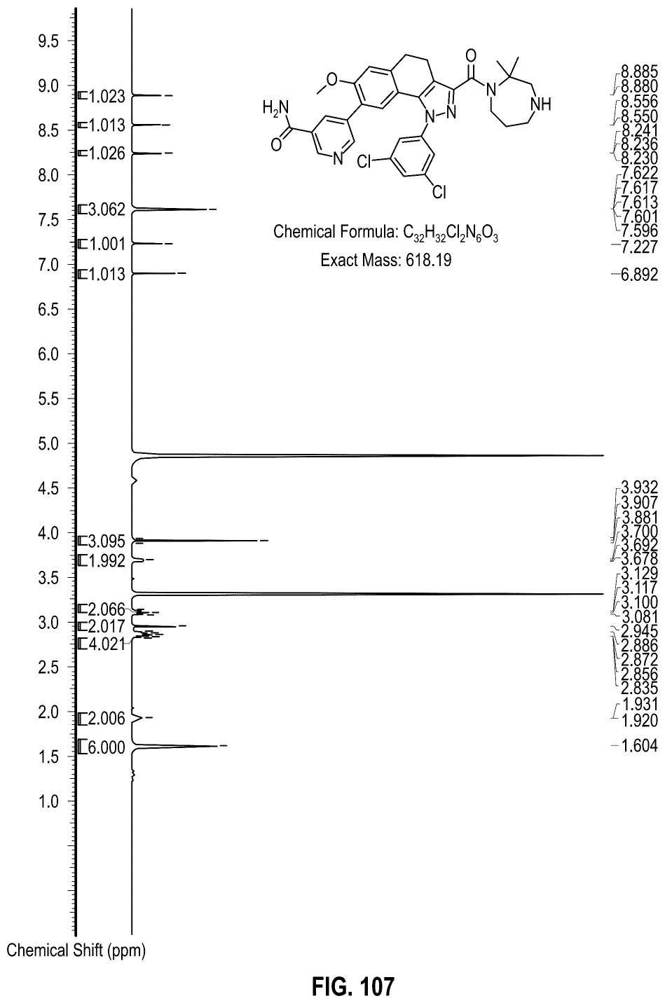

To a solution of benzyl 4-[8-(5-carbamoyl-3-pyridyl)-1-(3,5-dichlorophenyl)-7-methoxy-4,5-dihydrobenzo[g]indazole-3-carbonyl]-3,3-dimethyl-1,4-diazepane-1-carboxylate (170 mg, 225.56 μmol, 1 eq) in ACN (5 mL) was added TMSI (112.83 mg, 563.91 μmol, 76.76 μL, 2.5 eq) at 0° C. The mixture was stirred at 25° C. for 16.5 hr. The mixture was basified to pH=8˜9 with saturated NaHCO3 aqueous solution. The aqueous layer was extracted with ethyl acetate (20 mL*3). The combined organic layers were washed with brine (20 mL), dried over Na2SO4, filtered and concentrated in vacuum to give a residue. The residue was purified by prep-HPLC (column: C18 150×30 mm; mobile phase: [water (FA)-ACN]; gradient: 20%-50% B over 7 min), followed by lyophilization. The crude product was purified by prep-HPLC (column: Waters Xbridge 150*25 mm*5 um; mobile phase:[water (NH4HCO3)-ACN]; gradient: 32%-62% B over 9 min), followed by lyophilization. Compound 5-[1-(3,5-dichlorophenyl)-3-(2,2-dimethyl-1,4-diazepane-1-carbonyl)-7-methoxy-4,5-dihydrobenzo[g]indazol-8-yl]pyridine-3-carboxamide (2.43 mg, 3.92 μmol, 1.74% yield, 100% purity) was obtained as a white solid. LCMS (ESI): m/z[M+H] calcd for C32H33Cl2N6O3: 619.19; found:619.3. 1H NMR (400 MHz, METHANOL-d4) δ=8.88 (d, J=2.1 Hz, 1H), 8.55 (d, J=2.1 Hz, 1H), 8.24 (t, J=2.1 Hz, 1H), 7.65-7.56 (m, 3H), 7.23 (s, 1H), 6.89 (s, 1H), 3.91 (s, 3H), 3.75-3.62 (m, 2H), 3.14-3.06 (m, 2H), 2.94 (s, 2H), 2.90-2.79 (m, 4H), 1.93 (br d, J=4.3 Hz, 2H), 1.60 (s, 6H). FIG. 107 shows the nuclear magnetic resonance of Compound 8-4.

### Method B

To a solution of benzyl 4-[8-(5-carbamoyl-3-pyridyl)-1-(3,5-dichlorophenyl)-7-methoxy-4,5-dihydrobenzo[g]indazole-3-carbonyl]-3,3-dimethyl-1,4-diazepane-1-carboxylate (40 mg, 53.07 μmol, 1 eq) in AcOH (1 mL) was added hydrogen bromide (1.49 g, 6.81 mmol, 1 mL, 37% purity, 128.38 eq) at 0° C. The mixture was stirred at 0° C. for 1 hr. The mixture was basified to pH=8-9 with saturated NaHCO₃ aqueous solution. The aqueous layer was extracted with ethyl acetate (10 mL*3). The combined organic layers were washed with brine (10 mL), dried over Na₂SO₄, filtered and concentrated in vacuum to give a residue. The residue was purified by prep-HPLC (column: Waters Xbridge 150*25 mm*5 um; mobile phase:[water (NH₄HCO₃)-ACN]; gradient: 32%-62% B over 9 min), followed by lyophilization. Compound 5-[1-(3,5-dichlorophenyl)-3-(2,2-dimethyl-1, 4-diazepane-1-carbonyl)-7-methoxy-4,5-dihydrobenzo[g]indazol-8-yl]pyridine-3-carboxamide (17 mg, 27.44 μmol, 51.70% yield) was obtained as a white solid.

### Synthesis of Compound 8-8

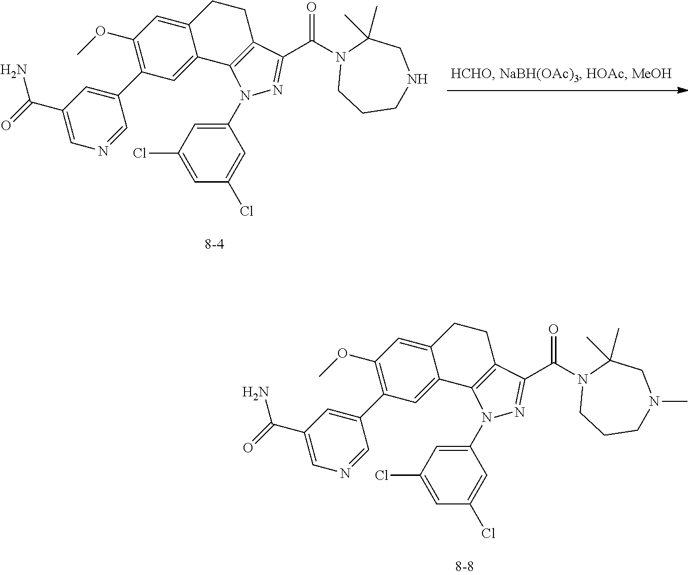 [SMILES: COc1cc2c(cc1-c1cncc(C(N)=O)c1)-c1c(c(C(=O)N3CCCNCC3(C)C)nn1-c1cc(Cl)cc(Cl)c1)CC2 ; COc1cc2c(cc1-c1cncc(C(N)=O)c1)-c1c(c(C(=O)N3CCCN(C)CC3(C)C)nn1-c1cc(Cl)cc(Cl)c1)CC2]

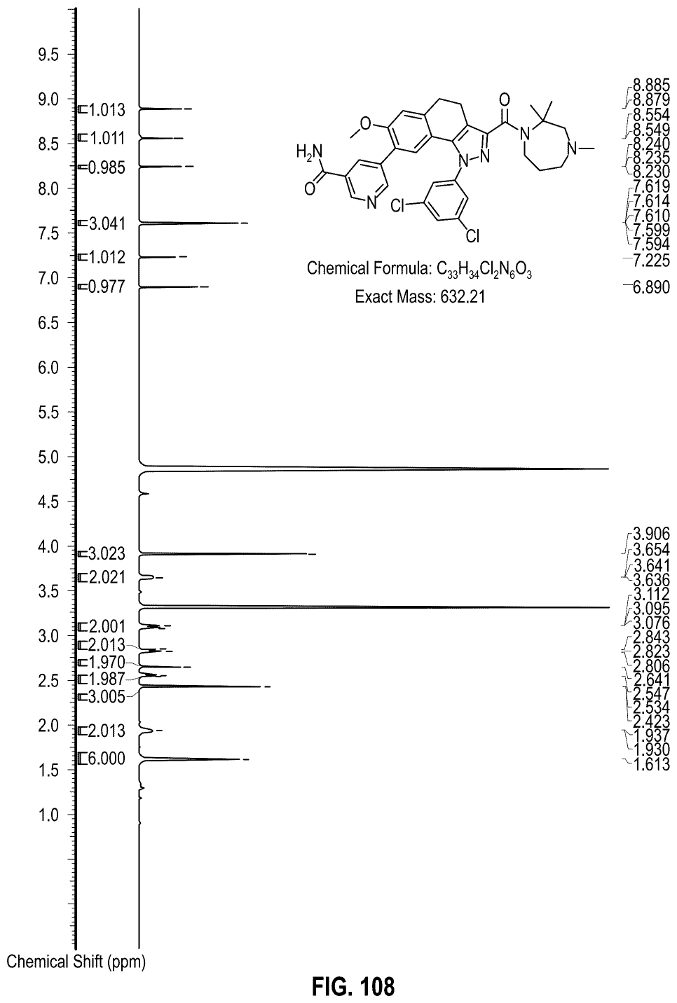

To a mixture of 5-[1-(3,5-dichlorophenyl)-3-(2,2-dimethyl-1,4-diazepane-1-carbonyl)-7-methoxy-4,5-dihydrobenzo[g]indazol-8-yl]pyridine-3-carboxamide (17 mg, 27.44 μmol, 1 eq) in MeOH (1 mL) was added AcOH (329.56 pg, 5.49 μmol, 3.14e-1 μL, 0.2 eq) and HCHO (2.67 mg, 32.93 μmol, 2.45 μL, 1.2 eq). The mixture was stirred at 25° C. for 1 h. Then NaBH (OAc)3 (17.45 mg, 82.32 μmol, 3 eq) was added. The mixture was stirred at 25° C. for 16 h. The reaction mixture was concentrated under reduced pressure to give a residue. The residue was purified by prep-HPLC (column: Waters Xbridge 150*25 mm*5 um; mobile phase: [water (NH4HCO3)-ACN]; gradient: 45%-75% B over 9 min), followed by lyophilization. Compound 5-[1-(3,5-dichlorophenyl)-7-methoxy-3-(2,2,4-trimethyl-1,4-diazepane-1-carbonyl)-4,5-dihydrobenzo[g]indazol-8-yl]pyridine-3-carboxamide (4.62 mg, 7.29 μmol, 26.57% yield, 100% purity) was obtained as a white solid. LCMS (ESI): m/z[M+H] calcd for C33H35Cl2N6O3: 633.21; found:633.3. 1H NMR (400 MHz, METHANOL-d4) δ=8.88 (d, J=2.1 Hz, 1H), 8.55 (d, J=2.1 Hz, 1H), 8.24 (t, J=2.1 Hz, 1H), 7.64-7.57 (m, 3H), 7.22 (s, 1H), 6.89 (s, 1H), 3.91 (s, 3H), 3.69-3.59 (m, 2H), 3.09 (t, J=7.2 Hz, 2H), 2.87-2.79 (m, 2H), 2.64 (s, 2H), 2.54 (br d, J=5.3 Hz, 2H), 2.42 (s, 3H), 1.93 (br d, J=2.9 Hz, 2H), 1.61 (s, 6H). FIG. 108 shows the nuclear magnetic resonance of Compound 8-8.

### Synthesis of 2,2-dimethyl-1,4-diazepan-5-one

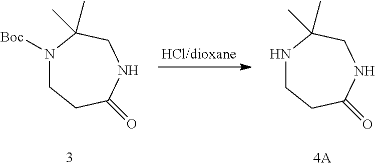

A solution of tert-butyl 2,2-dimethyl-5-oxo-1,4-diazepane-1-carboxylate (50 mg, 206.34 μmol, 1 eq) in HCl/dioxane (2 M, 2 mL, 19.39 eq) was stirred at 25° C. for 2 hr. The reaction mixture was concentrated under reduced pressure to give a residue. The crude product 2,2-dimethyl-1,4-diazepan-5-one (36 mg, crude, HCl) as a white solid was used into the next step without further purification.

### Synthesis of Compound 8-4A

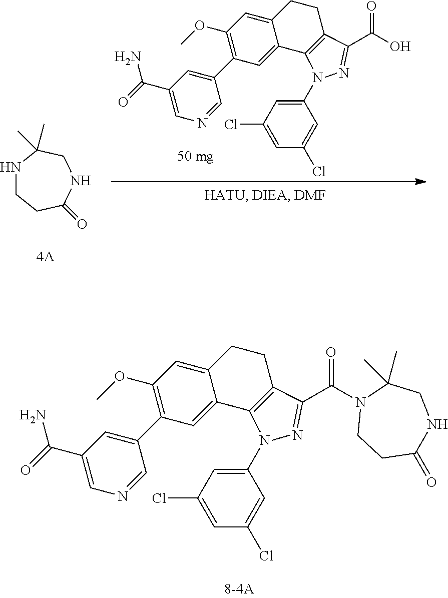

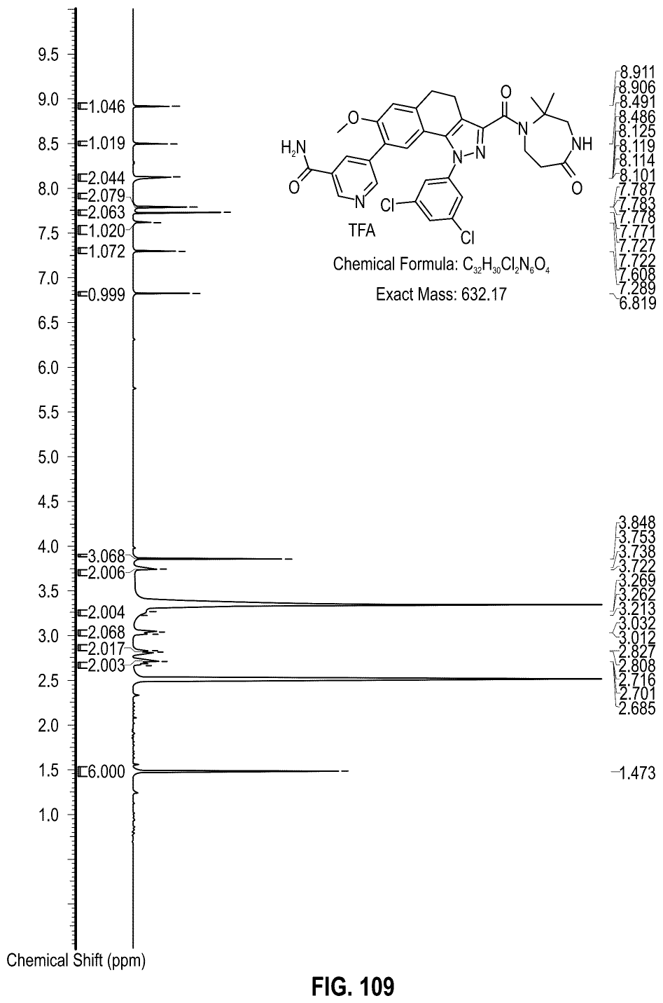

To a solution of 8-(5-carbamoyl-3-pyridyl)-1-(3,5-dichlorophenyl)-7-methoxy-4,5-dihydrobenzo[g]indazole-3-carboxylic acid (50 mg, 98.17 μmol, 1 eq) in DMF (1 mL) was added HATU (44.79 mg, 117.80 μmol, 1.2 eq) and DIEA (38.06 mg, 294.50 μmol, 51.30 μL, 3 eq). The mixture stirred at 25° C. for 30 min. Then to the mixture was added a mixture of 2,2-dimethyl-1,4-diazepan-5-one (31.57 mg, 176.70 μmol, 1.8 eq, HCl) in DMF (1 mL). The mixture stirred at 25° C. for 16 hr. The reaction mixture was concentrated under reduced pressure to give a residue. The residue was purified by prep-HPLC (column: Phenomenex Luna C18 150*25 mm*10 um; mobile phase: [water (TFA)-ACN]; gradient: 30%-60% B over 15 min), followed by lyophilization. Compound 5-[1-(3,5-dichlorophenyl)-3-(2,2-dimethyl-5-oxo-1,4-diazepane-1-carbonyl)-7-methoxy-4,5-dihydrobenzo[g]indazol-8-yl]pyridine-3-carboxamide (32.69 mg, 43.73 μmol, 44.55% yield, 100% purity, TFA) was obtained as a light yellow solid. LCMS (ESI): m/z[M+H] calcd for C32H31Cl2N6O4: 633.17; found:633.3. 1H NMR (400 MHz, DMSO-d6) δ=8.91 (d, J=2.0 Hz, 1H), 8.49 (d, J=2.1 Hz, 1H), 8.15-8.08 (m, 2H), 7.81-7.75 (m, 2H), 7.72 (d, J=1.8 Hz, 2H), 7.61 (s, 1H), 7.29 (s, 1H), 6.82 (s, 1H), 3.85 (s, 3H), 3.74 (br t, J=6.3 Hz, 2H), 3.28-3.21 (m, 2H), 3.03 (br t, J=7.4 Hz, 2H), 2.85-2.78 (m, 2H), 2.70 (br t, J=6.2 Hz, 2H), 1.47 (s, 6H). FIG. 109 shows the nuclear magnetic resonance of Compound 8-4a.

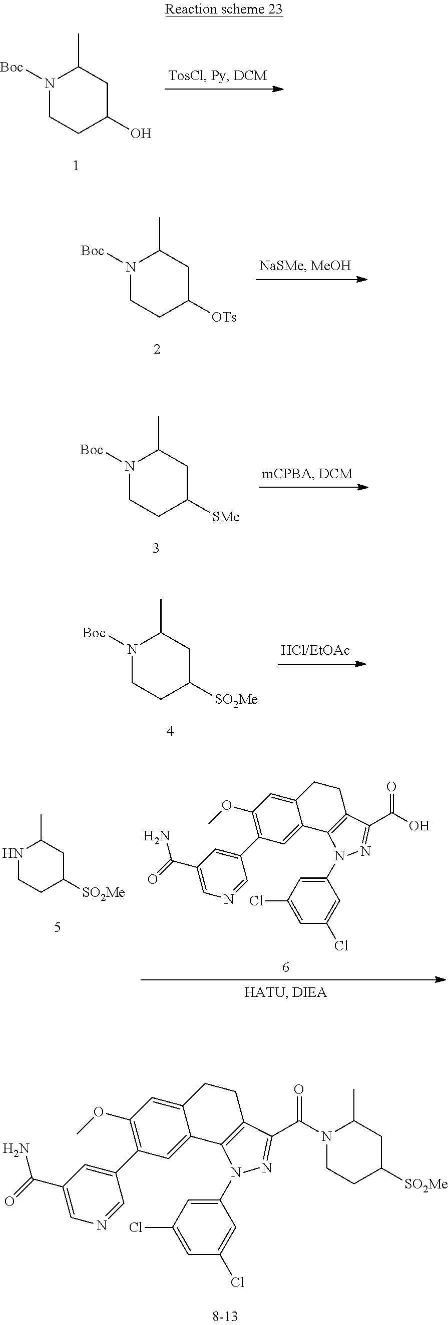

### Synthesis of tert-butyl 2-methyl-4-(tosyloxy)piperidine-1-carboxylate

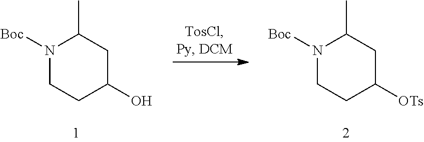 [SMILES: CC1CC(O)CCN1C(=O)OC(C)(C)C ; Cc1ccc(S(=O)(=O)OC2CCN(C(=O)OC(C)(C)C)C(C)C2)cc1]

A mixture of tert-butyl 4-hydroxy-2-methyl-piperidine-1-carboxylate (2 g, 9.29 mmol, 1 eq), TosCl (2.30 g, 12.08 mmol, 1.3 eq), Py (1.47 g, 18.58 mmol, 1.50 mL, 2 eq) in DCM (20 mL), and then the mixture was stirred at 25° C. for 16 hr. The mixture was poured into water (40 mL) and extracted with ethyl acetate (3*20 mL). The organic phase was separated, washed with Saturated sodium chloride solution (10 mL), dried over Na2SO4, filtered and concentrated under reduced pressure to give a residue. The residue was purified by flash silica gel chromatography (ISCO®; 25 g SepaFlash® Silica Flash Column, Eluent of 85˜75% Petroleum ether/Ethyl acetategradient @60 mL/min). Compound tert-butyl 2-methyl-4-(p-tolylsulfonyloxy)piperidine-1-carboxylate (1.4 g, 3.64 mmol, 39.16% yield, 96% purity) was obtained as a yellow oil. 1H NMR (400 MHz, DMSO-d6) δ=7.87-7.74 (m, 2H), 7.48 (d, J=8.0 Hz, 2H), 4.82-4.71 (m, 1H), 4.35-4.11 (m, 1H), 3.88-3.65 (m, 1H), 3.02-2.81 (m, 1H), 2.42 (s, 3H), 1.85-1.74 (m, 1H), 1.68-1.40 (m, 3H), 1.36 (d, J=3.2 Hz, 9H), 1.21-1.10 (m, 3H).

### Synthesis of tert-butyl 2-methyl-4-(methylthio)piperidine-1-carboxylate

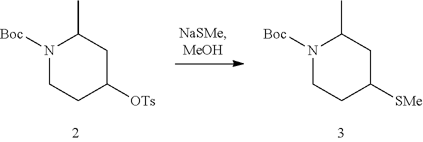 [SMILES: Cc1ccc(S(=O)(=O)OC2CCN(C(=O)OC(C)(C)C)C(C)C2)cc1 ; CSC1CCN(C(=O)OC(C)(C)C)C(C)C1]

To a solution of tert-butyl 2-methyl-4-(p-tolylsulfonyloxy)piperidine-1-carboxylate (1.4 g, 3.79 mmol, 1 eq) in EtOH (15 mL) was added NaSMe (531.16 mg, 7.58 mmol, 482.87 μL, 2 eq).The mixture was stirred at 80° C. for 16hrunder N2 atomasphere. The mixture was poured into water (30 mL) and extracted with ethyl acetate (3*20 mL). The organic phase was separated, washed with Saturated sodium chloride solution (10 mL), dried over Na2SO4, filtered and concentrated under reduced pressure to give a residue. The residue was purified by flash silica gel chromatography (ISCO®; 12 g SepaFlash® Silica Flash Column, Eluent of 80˜75% Petroleum ether/Ethyl acetategradient @60 m/min). Compound tert-butyl 2-methyl-4-methylsulfanyl-piperidine-1-carboxylate (500 mg, 1.94 mmol, 51.09% yield, 95% purity) was obtained as a colorless oil. 1H NMR (400 MHz, DMSO-d6) δ=4.07-3.94 (m, 1H), 3.70-3.59 (m, 1H), 3.13 (m, 1H), 2.96-2.87 (m, 1H), 2.06-2.04 (m, 3H), 2.02-1.96 (m, 1H), 1.88-1.71 (m, 1H), 1.63-1.49 (m, 2H), 1.39 (s, 9H), 1.19 (d, J=6.8 Hz, 3H).

### Synthesis of tert-butyl 2-methyl-4-(methylsulfonyl)piperidine-1-carboxylate

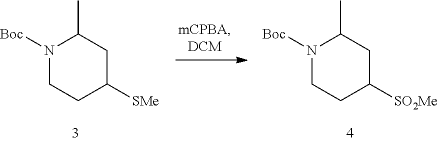 [SMILES: CSC1CCN(C(=O)OC(C)(C)C)C(C)C1 ; CC1CC(S(C)(=O)=O)CCN1C(=O)OC(C)(C)C]

To a solution of tert-butyl 2-methyl-4-methylsulfanyl-piperidine-1-carboxylate (500 mg, 2.04 mmol, 1 eq) in DCM (5 mL) was added m-CPBA (1.03 g, 5.09 mmol, 85% purity, 2.5 eq). The mixture was stirred at 25° C. for 2 hr The reaction was quenched by addition of 10 mL of sodium sulfite solution and extracted with ethyl acetate(3*10 mL). The organic phase was separated, washed with Saturated sodium chloride solution (5 mL), dried over Na2SO4, filtered and concentrated under reduced pressure to give a residue. The residue was purified by flash silica gel chromatography (ISCO®;25 g SepaFlash® Silica Flash Column, Eluent of 25-40% Petroleum ether/Ethyl acetategradient @60 mL/min). Compound tert-butyl 2-methyl-4-methylsulfonyl-piperidine-1-carboxylate (440 mg, 1.59 mmol, 77.85% yield) was obtained as a colorless oil. 1H NMR (400 MHz, DMSO-d6) δ=3.87-3.74 (m, 1H), 3.65 (m, 1H), 3.31-3.24 (m, 1H), 3.20-3.07 (m, 1H), 2.96-2.89 (m, 3H), 2.18-2.06 (m, 1H), 1.95 (s, 1H), 1.82-1.70 (m, 1H), 1.53 (m, 1H), 1.40 (s, 9H), 1.20-1.14 (m, 3H).

### Synthesis of 2-methyl-4-(methylsulfonyl)piperidine

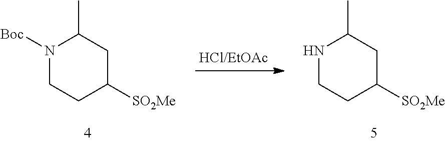 [SMILES: CC1CC(S(C)(=O)=O)CCN1C(=O)OC(C)(C)C ; CC1CC(S(C)(=O)=O)CCN1]

To a solution of tert-butyl 2-methyl-4-(methylsulfonyl)piperidine-1-carboxylate (440 mg, 1.59 mmol, 1 eq) in HCl/dioxane (5 mL).The mixture was stirred at 25° C. for 2 hr. The mixture was was concentrated to give a residue. Compound 2-methyl-4-(methylsulfonyl)piperidine (260 mg, 1.22 mmol, 76.69% yield, HCl) was obtained as a white solid. 1H NMR (400 MHz, DMSO-d6) δ=9.68-9.44 (m, 1H), 9.07-8.81 (m, 1H), 3.51-3.38 (m, 2H), 3.16 (d, J=8.0 Hz, 1H), 2.98 (s, 3H), 2.90 (d, J=11.6 Hz, 1H), 2.25-2.13 (m, 2H), 1.86-1.71 (m, 1H), 1.61 (q, J=12.8 Hz, 1H), 1.30 (d, J=6.4 Hz, 3H).

### Synthesis of Compound 8-13

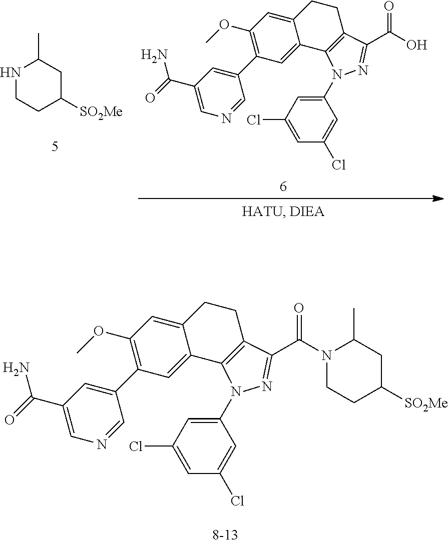 [SMILES: CC1CC(S(C)(=O)=O)CCN1 ; COc1cc2c(cc1-c1cncc(C(N)=O)c1)-c1c(c(C(=O)O)nn1-c1cc(Cl)cc(Cl)c1)CC2 ; COc1cc2c(cc1-c1cncc(C(N)=O)c1)-c1c(c(C(=O)N3CCC(S(C)(=O)=O)CC3C)nn1-c1cc(Cl)cc(Cl)c1)CC2]

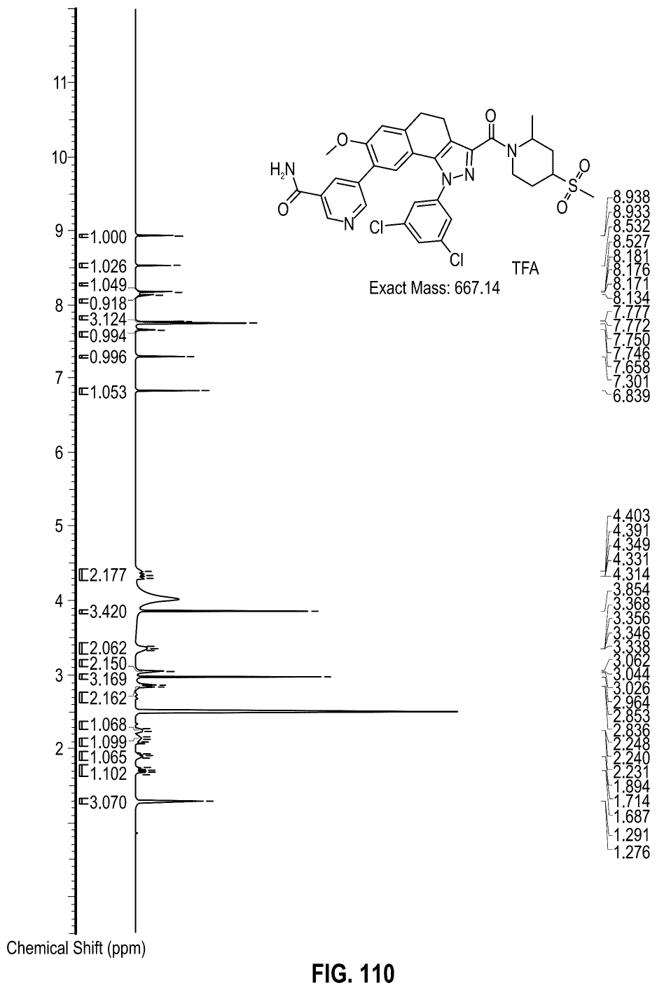

A mixture of 8-(5-carbamoyl-3-pyridyl)-1-(3,5-dichlorophenyl)-7-methoxy-4,5-dihydrobenzo[g]indazole-3-carboxylic acid (50 mg, 98.17 μmol, 1 eq), 2-methyl-4-methylsulfonyl-piperidine (19.14 mg, 107.98 μmol, 1.1 eq), HATU (55.99 mg, 147.25 μmol, 1.5 eq), DIEA (38.06 mg, 294.50 μmol, 51.30 μL, 3 eq) in DMF (1 mL) was stirred at 25° C. for 2 hr. The pH of the reaction mixture was adjusted to 7 with 1 M HCl. Compound 5-[1-(3,5-dichlorophenyl)-7-methoxy-3-(2-methyl-4-methylsulfonyl-piperidine-1-carbonyl)-4,5-dihydrobenzo[g]indazol-8-yl]pyridine-3-carboxamide (35.37 mg, 44.74 μmol, 45.58% yield, 99% purity, TFA) was obtained as a white solid. LCMS (ESI): m/z[M+H] calcd for C₃₂H₃₁N₅O₄Cl₂S: 668.14; found: 668.1. ¹H NMR (400 MHz, DMSO-d6) δ=8.94 (d, J=2.0 Hz, 1H), 8.53 (d, J=2.0 Hz, 1H), 8.18 (t, J=2.0 Hz, 1H), 8.13 (s, 1H), 7.79-7.73 (m, 3H), 7.66 (s, 1H), 7.30 (s, 1H), 6.84 (s, 1H), 4.43-4.28 (m, 2H), 3.85 (s, 3H), 3.43-3.28 (m, 2H), 3.11-3.00 (m, 2H), 2.96 (s, 3H), 2.91-2.78 (m, 2H), 2.24 (m, 1H), 2.20-2.08 (m, 1H), 1.89 (m, 1H), 1.79-1.62 (m, 1H), 1.28 (d, J=6.0 Hz, 3H). FIG. 110 shows the nuclear magnetic resonance of Compound 8-13.

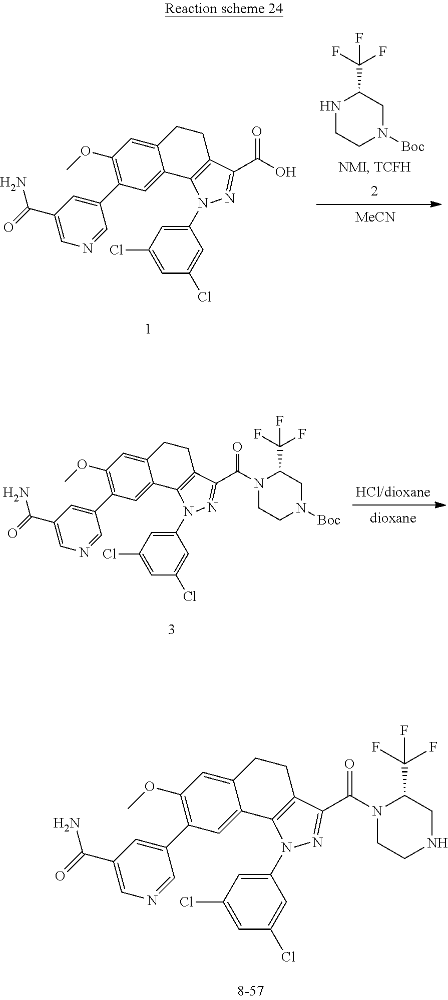 [SMILES: "MeCN" ; COc1cc2c(cc1-c1cncc(C(N)=O)c1)-c1c(c(C(=O)O)nn1-c1cc(Cl)cc(Cl)c1)CC2 ; CC(C)(C)OC(=O)N1CCN[C@H](C(F)(F)F)C1 ; COc1cc2c(cc1-c1cncc(C(N)=O)c1)-c1c(c(C(=O)N3CCN(C(=O)OC(C)(C)C)C[C@H]3C(F)(F)F)nn1-c1cc(Cl)cc(Cl)c1)CC2 ; COc1cc2c(cc1-c1cncc(C(N)=O)c1)-c1c(c(C(=O)N3CCNC[C@H]3C(F)(F)F)nn1-c1cc(Cl)cc(Cl)c1)CC2]

### Synthesis of tert-butyl(3S)-4-[8-(5-carbamoyl-3-pyridyl)-1-(3,5-dichlorophenyl)-7-methoxy-4,5-dihydrobenzo[g]indazole-3-carbonyl]-3-(trifluoromethyl)piperazine-1-carboxylate

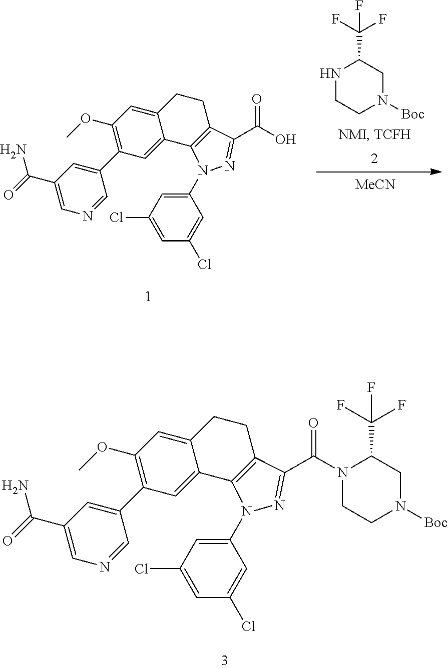 [SMILES: "MeCN" ; COc1cc2c(cc1-c1cncc(C(N)=O)c1)-c1c(c(C(=O)O)nn1-c1cc(Cl)cc(Cl)c1)CC2 ; CC(C)(C)OC(=O)N1CCN[C@H](C(F)(F)F)C1 ; COc1cc2c(cc1-c1cncc(C(N)=O)c1)-c1c(c(C(=O)N3CCN(C(=O)OC(C)(C)C)C[C@H]3C(F)(F)F)nn1-c1cc(Cl)cc(Cl)c1)CC2]

To a solution of 8-(5-carbamoyl-3-pyridyl)-1-(3,5-dichlorophenyl)-7-methoxy-4,5-dihydrobenzo[g]indazole-3-carboxylic acid (50 mg, 98.17 μmol, 1 eq), tert-butyl (3S)-3-(trifluoromethyl)piperazine-1-carboxylate (29.95 mg, 117.80 μmol, 1.2 eq) and NMI (8.87 mg, 107.98 μmol, 8.61 μL, 1.1 eq) in MeCN (1 mL) was added TCFH (57.84 mg, 206.15 μmol, 2.1 eq) in one portion. The mixture was stirred at 50° C. for 3 hours. The reaction mixture was diluted with water (5 ml), and then extracted with EtOAc (5 mL*3). The combined organic layers were washed with brine (5 mL*2), dried over anhydrous Na₂SO₄, filtered and the filtrate was concentrated under reduced pressure to afford tert-butyl (3S)-4-[8-(5-carbamoyl-3-pyridyl)-1-(3,5-dichlorophenyl)-7-methoxy-4,5-dihy drobenzo[g]indazole-3-carbonyl]-3-(trifluoromethyl)piperazine-1-carboxylate (53 mg, crude) as a brown solid.

### Synthesis of Compound 8-57

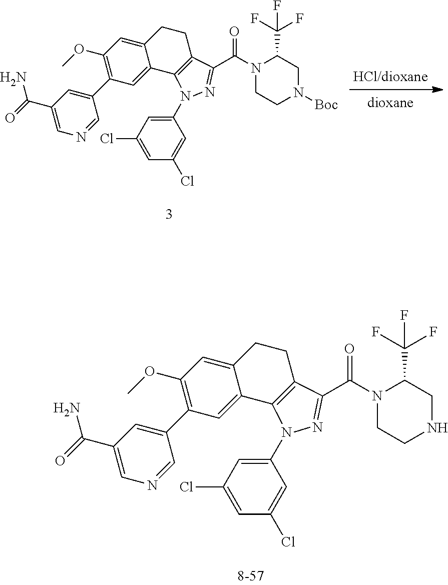 [SMILES: COc1cc2c(cc1-c1cncc(C(N)=O)c1)-c1c(c(C(=O)N3CCN(C(=O)OC(C)(C)C)C[C@H]3C(F)(F)F)nn1-c1cc(Cl)cc(Cl)c1)CC2 ; COc1cc2c(cc1-c1cncc(C(N)=O)c1)-c1c(c(C(=O)N3CCNC[C@H]3C(F)(F)F)nn1-c1cc(Cl)cc(Cl)c1)CC2]

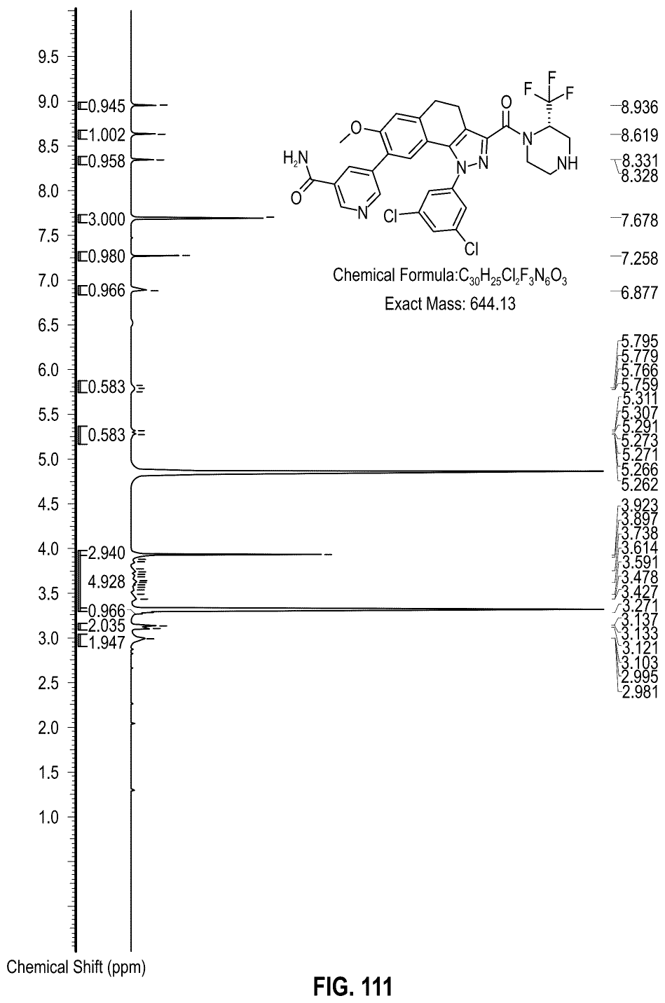

To a solution of tert-butyl(3S)-4-[8-(5-carbamoyl-3-pyridyl)-1-(3,5-dichlorophenyl)-7-methoxy-4,5-dihydrobenzo[g]indazole-3-carbonyl]-3-(trifluoromethyl)piperazine-1-carboxylate (53 mg, 71.09 μmol, 1 eq) in dioxane (1 mL) was added HCl/dioxane (2 M, 355.43 μL, 10 eq) in one portion. The mixture was stirred at 25° C. for 1 hour. The reaction mixture was concentrated under reduced pressure to give a residue. The residue was purified by prep-HPLC (column: Phenomenex Luna C18 150*25 mm*10 um;mobile phase:[water(TFA)-ACN];gradient:22%-52% B over 9 min), followed by lyophilization to afford 5-[1-(3,5-dichlorophenyl)-7-methoxy-3-[(2S)-2-(trifluoromethyl)piperazine-1-carbonyl]-4,5-dihydrobenzo[g]indazol-8-yl]pyridine-3-carboxamide (3.77 mg, 4.96 μmol, 6.98% yield, 100% purity, TFA) as a yellow solid. LCMS (ESI): m z[M+H] calcd for C₃₀H₂₆Cl₂F₃N₆O3: 645.13; found: 645.3. ¹H NMR (400 MHz, METHANOL-d4) δ=8.94 (s, 1H), 8.62 (s, 1H), 8.33 (d, J=1.4 Hz, 1H), 7.68 (s, 3H), 7.26 (s, 1H), 6.88 (br s, 1H), 5.86-5.72 (m, 1H), 5.36-5.16 (m, 1H), 3.92 (s, 3H), 3.91-3.32 (m, 5H), 3.27 (br s, 1H), 3.16-3.08 (m, 2H), 2.99 (br d, J=5.3 Hz, 2H). FIG. 111 shows the nuclear magnetic resonance of Compound 8-57.

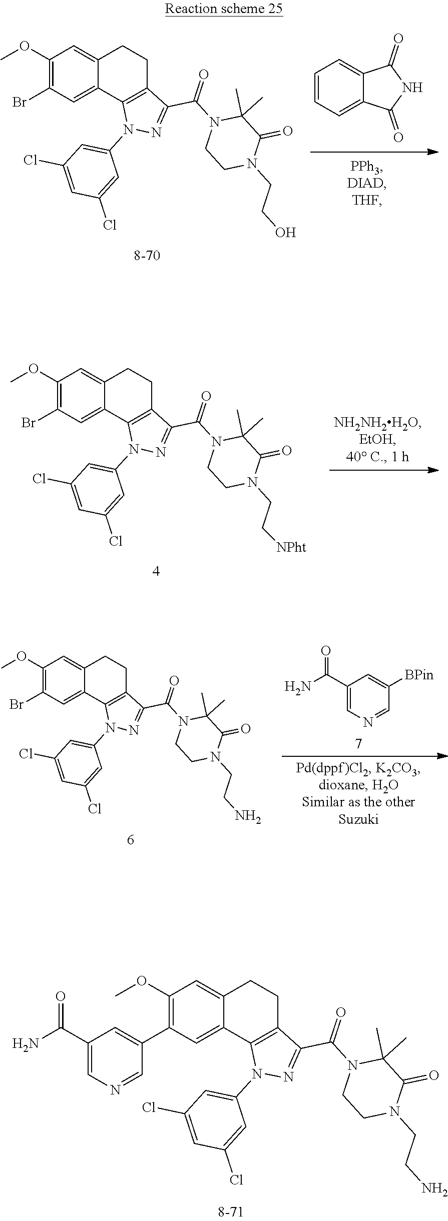

### Synthesis of 2-[2-[4-[8-bromo-1-(3,5-dichlorophenyl)-7-methoxy-4,5-dihydrobenzo[g]indazole-3-carbonyl]-3,3-dimethyl-2-oxo-piperazin-1-yl]ethyl]isoindoline-1,3-dione

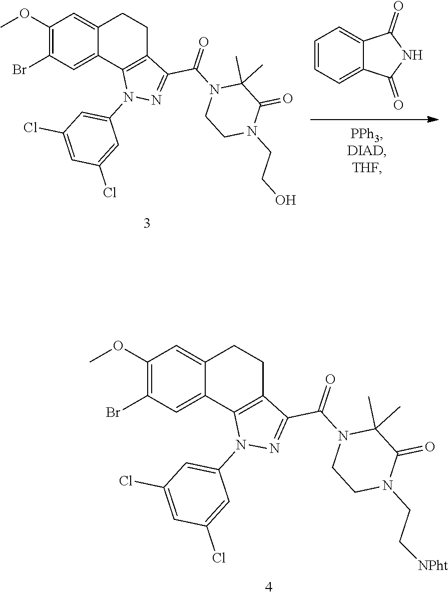

To a solution of 4-[8-bromo-1-(3,5-dichlorophenyl)-7-methoxy-4,5-dihydrobenzo[g]indazole-3-carbonyl]-1-(2-hydroxyethyl)-3,3-dimethyl-piperazin-2-one (60 mg, 96.41 μmol, 1 eq), isoindoline-1,3-dione (17.02 mg, 115.69 μmol, 1.2 eq) and PPh3 (32.87 mg, 125.33 μmol, 1.3 eq) in THF (2 mL) was added DIAD (23.39 mg, 115.69 μmol, 22.43 μL, 1.2 eq) at 0° C. under N2 atmosphere. The mixture was stirred at 60° C. for 20 hr. The reaction mixture was diluted with ice water (20 mL). The aqueous Layer was extracted with ethyl acetate (20 mL*3). The combined organic Layers were dried with anhydrous Na2SO4, filtered and concentrated in vacuum to give a residue. The residue was purified by prep-HPLC (column: Phenomenex Luna C18 150*25 mm*10 um; mobile phase:[water (FA)-ACN]; gradient: 70%-100% B over 8 min). Compound 2-[2-[4-[8-bromo-1-(3, 5-dichlorophenyl)-7-methoxy-4,5-dihydrobenzo[g]indazole-3-carbonyl]-3,3-dimethyl-2-oxo-piperazin-1-yl]ethyl]isoindoline-1,3-dione (60 mg, 79.85 μmol, 82.82% yield) was obtained as a yellow oil. 1H NMR (400 MHz, CHLOROFORM-d) δ=7.84 (dd, J=3.1, 5.4 Hz, 2H), 7.74-7.68 (m, 2H), 7.49 (t, J=1.8 Hz, 1H), 7.43 (d, J=1.9 Hz, 2H), 7.06 (s, 1H), 6.89 (s, 1H), 4.05-3.98 (m, 2H), 3.97-3.90 (m, 5H), 3.74 (dd, J=4.3, 6.4 Hz, 2H), 3.61-3.54 (m, 2H), 3.00-2.87 (m, 4H), 1.65 (s, 6H).

### Synthesis of 1-(2-aminoethyl)-4-[8-bromo-1-(3,5-dichlorophenyl)-7-methoxy-4,5-dihydrobenzo[g]indazole-3-carbonyl]-3,3-dimethyl-piperazin-2-one

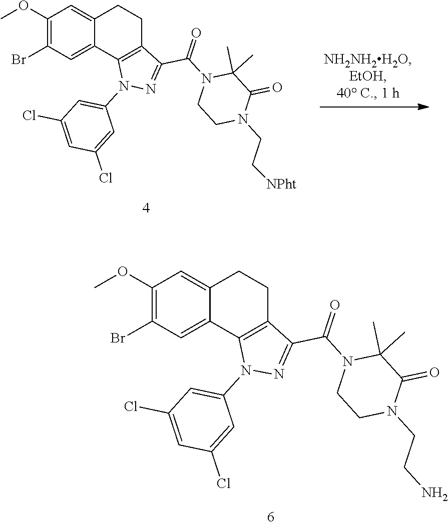

To a solution of 2-[2-[4-[8-bromo-1-(3,5-dichlorophenyl)-7-methoxy-4,5-dihydrobenzo[g]indazole-3-carbonyl]-3,3-dimethyl-2-oxo-piperazin-1-yl]ethyl]isoindoline-1,3-dione (60 mg, 79.85 μmol, 1 eq) in EtOH (1 mL) was added NH₂NH₂·H₂O (50 mg, 998.80 μmol, 48.45 μL, 12.51 eq). The mixture was stirred at 40° C. for 1 hr. The reaction mixture was filtered and concentrated under reduced pressure to give a residue. The crude product 1-(2-aminoethyl)-4-[8-bromo-1-(3,5-dichlorophenyl)-7-methoxy-4,5-dihydrobenzo[g]indazole-3-carbonyl]-3,3-dimethyl-piperazin-2-one (45 mg, crude) as a white solid was used into the next step without further purification.

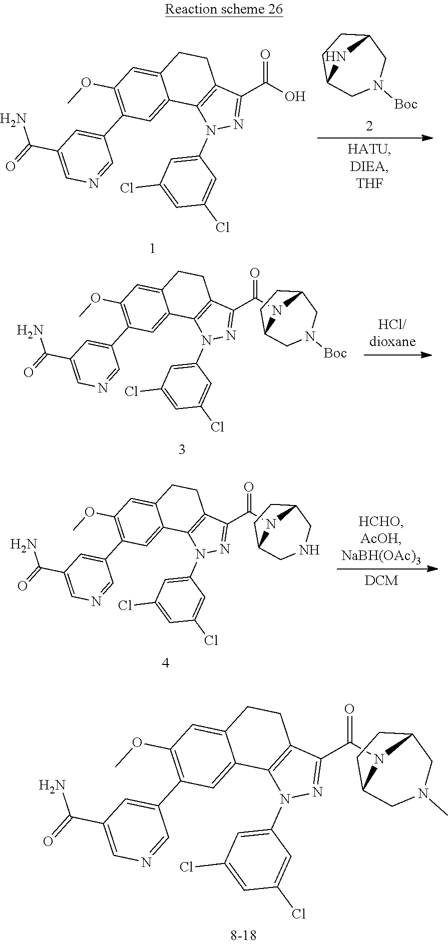 [SMILES: COc1cc2c(cc1-c1cncc(C(N)=O)c1)-c1c(c(C(=O)O)nn1-c1cc(Cl)cc(Cl)c1)CC2 ; CC(C)(C)OC(=O)N1C[C@H]2CC[C@@H](C1)N2 ; COc1cc2c(cc1-c1cncc(C(N)=O)c1)-c1c(c(C(=O)N3[C@@H]4CC[C@H]3CN(C(=O)OC(C)(C)C)C4)nn1-c1cc(Cl)cc(Cl)c1)CC2 ; COc1cc2c(cc1-c1cncc(C(N)=O)c1)-c1c(c(C(=O)N3[C@@H]4CC[C@H]3CNC4)nn1-c1cc(Cl)cc(Cl)c1)CC2 ; COc1cc2c(cc1-c1cncc(C(N)=O)c1)-c1c(c(C(=O)N3[C@@H]4CC[C@H]3CN(C)C4)nn1-c1cc(Cl)cc(Cl)c1)CC2]

### Synthesis of Compound 8-18

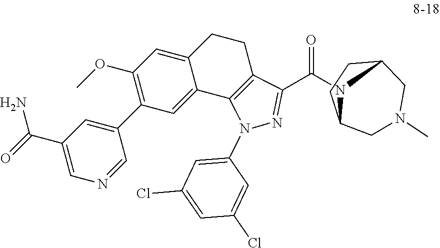 [SMILES: COc1cc2c(cc1-c1cncc(C(N)=O)c1)-c1c(c(C(=O)N3[C@@H]4CC[C@H]3CN(C)C4)nn1-c1cc(Cl)cc(Cl)c1)CC2]

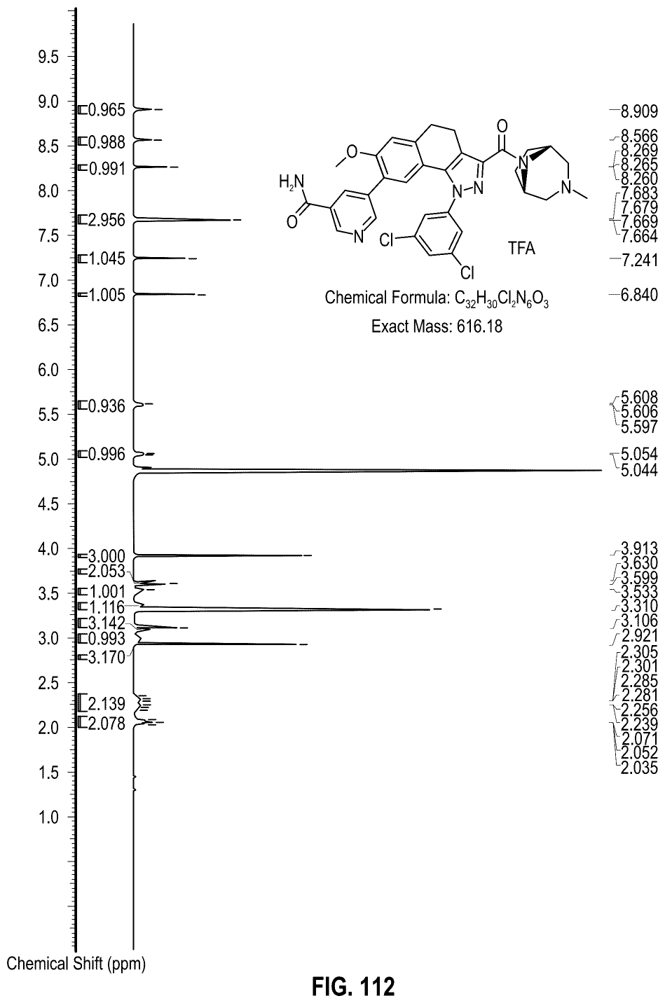

Compound 8-18 was synthesized according to a procedure similar to Reaction scheme 26. LCMS (ESI): m z[M+H] calcd for C₃₂H₃₁Cl₂N₆O₃: 617.18; found: 617.1. ¹H NMR (400 MHz, METHANOL-d4) δ=8.91 (s, 1H), 8.57 (br s, 1H), 8.26 (t, J=1.9 Hz, 1H), 7.70-7.64 (m, 3H), 7.24 (s, 1H), 6.84 (s, 1H), 5.64-5.56 (m, 1H), 5.05 (br d, J=4.3 Hz, 1H), 3.91 (s, 3H), 3.61 (br d, J=12.3 Hz, 2H), 3.53 (br s, 1H), 3.40-3.33 (m, 1H), 3.11 (br s, 3H), 3.04-2.95 (m, 1H), 2.92 (s, 3H), 2.36-2.17 (m, 2H), 2.05 (br t, J=7.3 Hz, 2H). FIG. 112 shows the nuclear magnetic resonance of Compound 8-18.

### Synthesis of Compound 8-35

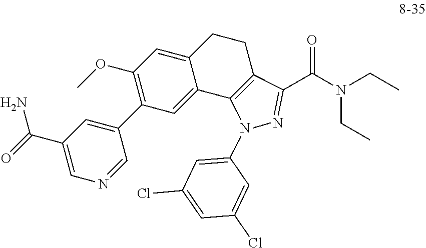 [SMILES: CCN(CC)C(=O)c1nn(-c2cc(Cl)cc(Cl)c2)c2c1CCc1cc(OC)c(-c3cncc(C(N)=O)c3)cc1-2]

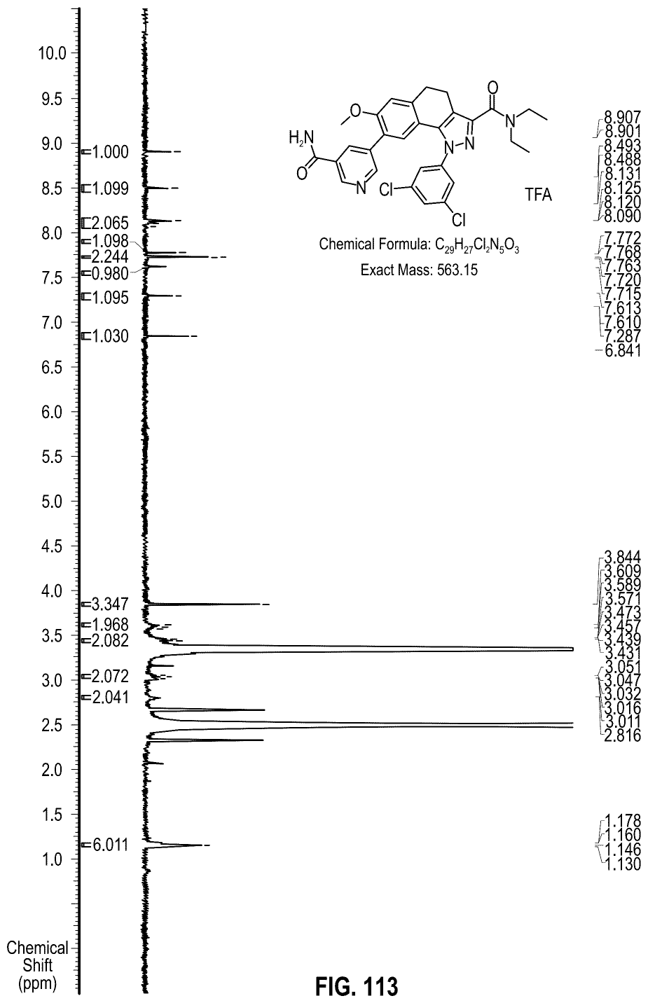

Compound 8-35 was synthesized according to a procedure similar to Reaction scheme 26. LCMS (ESI): m/z[M+H] calcd for C39H42N6O7Cl2F3: 564.15; found: 564.2. 1H NMR (400 MHz, METHANOL-d4) δ=9.00 (s, 1H), 8.75 (s, 1H), 8.52 (s, 1H), 7.63 (d, J=1.7 Hz, 3H), 7.51-7.45 (m, 1H), 7.35 (br d, J=8.1 Hz, 1H), 7.28 (s, 1H), 6.97 (d, J=4.5 Hz, 1H), 3.94 (s, 3H), 3.18-2.94 (m, 5H), 2.85 (q, J=7.0 Hz, 2H), 1.27 (br d, J=6.7 Hz, 6H). FIG. 113 shows the nuclear magnetic resonance of Compound 8-35.

### Synthesis of Compound 8-36

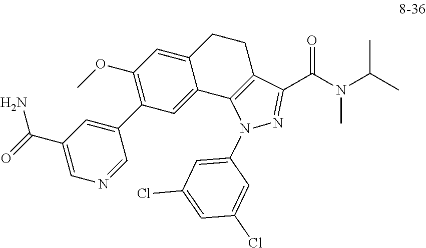 [SMILES: COc1cc2c(cc1-c1cncc(C(N)=O)c1)-c1c(c(C(=O)N(C)C(C)C)nn1-c1cc(Cl)cc(Cl)c1)CC2]

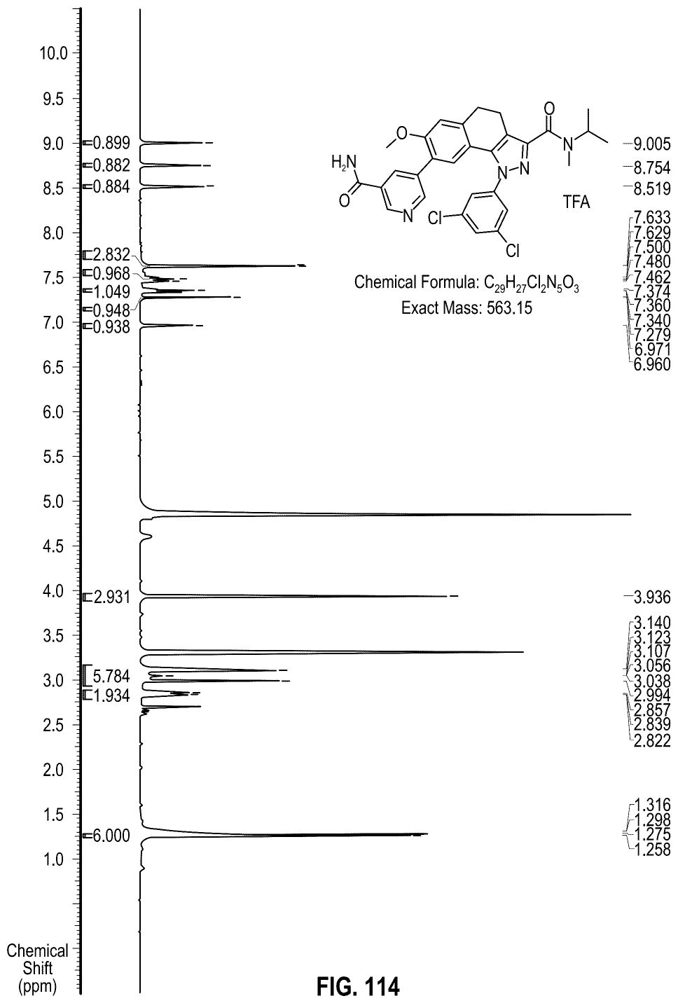

Compound 8-36 was synthesized according to a procedure similar to Reaction scheme 26. LCMS (ESI): m/z[M+H] calcd for C39H42N6O7Cl2F3: 564.15; found: 564.2. 1H NMR (400 MHz, METHANOL-d4) δ=9.00 (s, 1H), 8.75 (s, 1H), 8.52 (s, 1H), 7.63 (d, J=1.7 Hz, 3H), 7.51-7.45 (m, 1H), 7.35 (br d, J=8.1 Hz, 1H), 7.28 (s, 1H), 6.97 (d, J=4.5 Hz, 1H), 3.94 (s, 3H), 3.18-2.94 (m, 5H), 2.85 (q, J=7.0 Hz, 2H), 1.27 (br d, J=6.7 Hz, 6H). FIG. 114 shows the nuclear magnetic resonance of Compound 8-36.

### Synthesis of Compound 8-37

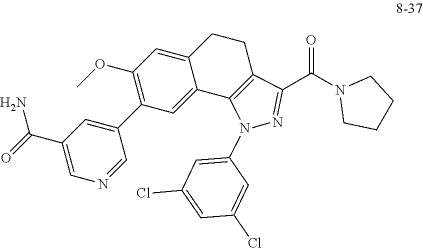 [SMILES: COc1cc2c(cc1-c1cncc(C(N)=O)c1)-c1c(c(C(=O)N3CCCC3)nn1-c1cc(Cl)cc(Cl)c1)CC2]

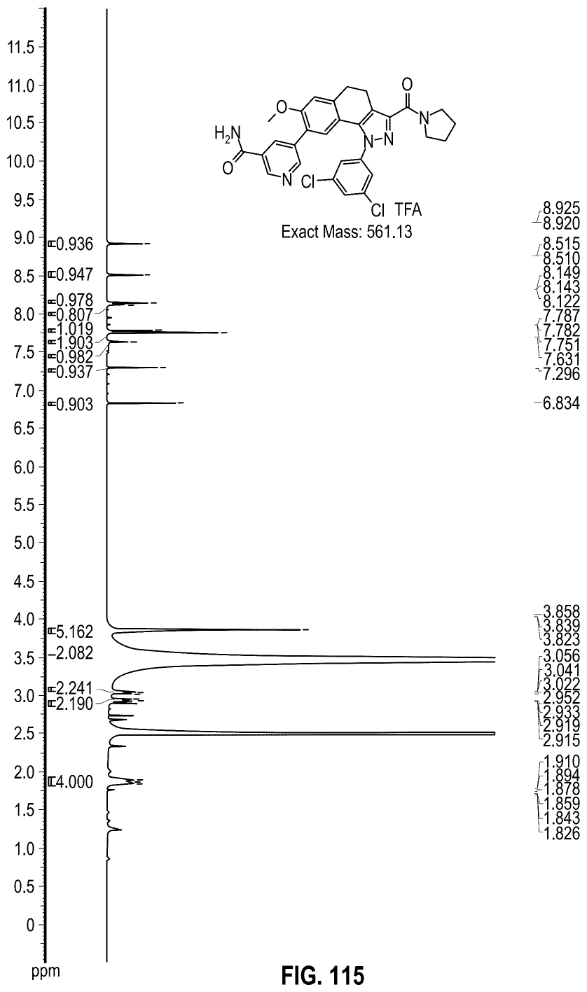

Compound 8-37 was synthesized according to a procedure similar to Reaction scheme 26. LCMS (ESI): m z[M+H] calcd for C₂₉H₂₅N₅O₃Cl₂ 562.45; found: 562.3. ¹H NMR (400 MHz, DMSO-d6) δ=8.92 (d, J=2.0 Hz, 1H), 8.51 (d, J=2.0 Hz, 1H), 8.15 (t, J=2.0 Hz, 1H), 8.12 (s, 1H), 7.80-7.77 (m, 1H), 7.75 (d, J=2.0 Hz, 2H), 7.63 (s, 1H), 7.30 (s, 1H), 6.83 (s, 1H), 3.87-3.81 (m, 5H), 3.53-3.52 (m, 2H), 3.06-3.01 (m, 2H), 2.97-2.91 (m, 2H), 1.93-1.82 (m, 4H). FIG. 115 shows the nuclear magnetic resonance of Compound 8-37.

### Synthesis of Compound 8-38

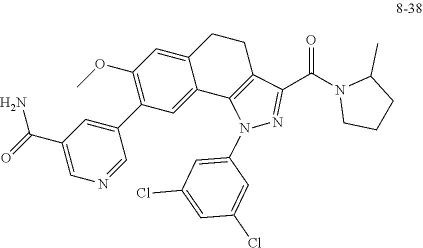 [SMILES: COc1cc2c(cc1-c1cncc(C(N)=O)c1)-c1c(c(C(=O)N3CCCC3C)nn1-c1cc(Cl)cc(Cl)c1)CC2]

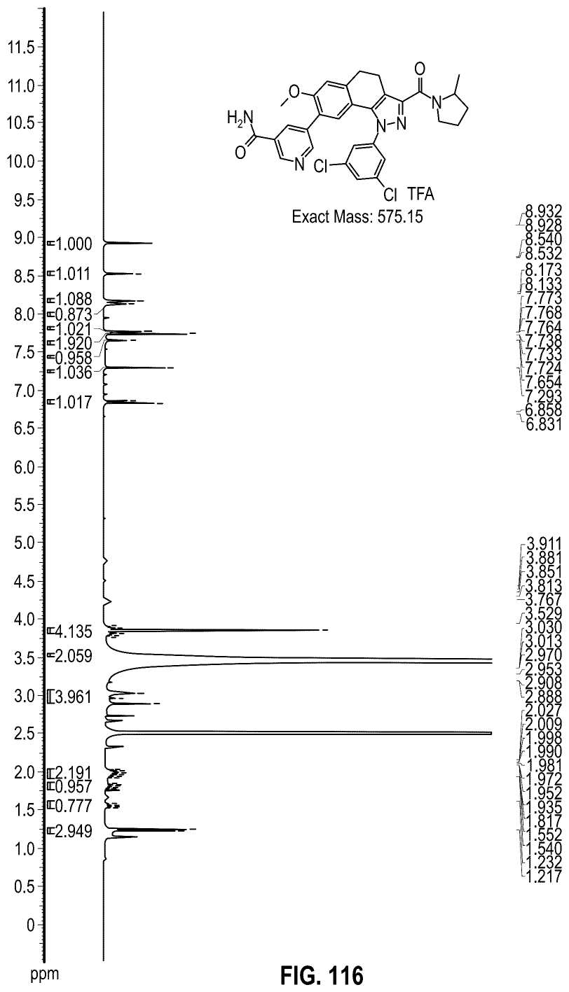

Compound 8-38 was synthesized according to a procedure similar to Reaction scheme 26. LCMS (ESI): m/z[M+H] calcd for C₃₀H₂₇N₅O₃Cl₂: 576.47; found: 576.3. 1H NMR (400 MHz, DMSO-d6) δ=8.93 (d, J=1.6 Hz, 1H), 8.56-8.51 (m, 1H), 8.20-8.16 (m, 1H), 8.13 (s, 1H), 7.79-7.75 (m, 1H), 7.75-7.71 (m, 2H), 7.65 (s, 1H), 7.29 (s, 1H), 6.87-6.82 (m, 1H), 3.88-3.80 (m, 4H), 3.53 (s, 2H), 3.07-2.90 (m, 4H), 2.04-1.91 (m, 2H), 1.81 (m, 1H), 1.61-1.51 (m, 1H), 1.22 (d, J=6.0 Hz, 3H). FIG. 116 shows the nuclear magnetic resonance of Compound 8-38.

### Synthesis of Compound 8-41

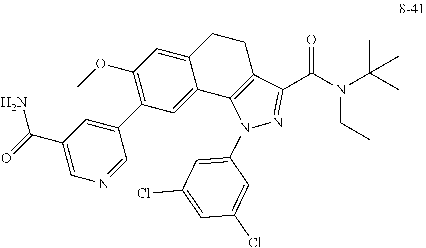 [SMILES: CCN(C(=O)c1nn(-c2cc(Cl)cc(Cl)c2)c2c1CCc1cc(OC)c(-c3cncc(C(N)=O)c3)cc1-2)C(C)(C)C]

Compound 8-41 trifluoroacetic acid salt and free base were synthesized according to a procedure similar to Reaction scheme 26.

### Trifluoroacetic Acid Salt of Compound 8-41

LCMS (ESI): m z[M+H] calcd for C₃₁H₃₁N₅O₃Cl₂: 592.51; found: 592.2. Batch 1(TFA salt): 1H NMR (400 MHz, METHANOL-d4) δ=8.88 (d, J=2.0 Hz, 1H), 8.55 (d, J=2.0 Hz, 1H), 8.23 (t, J=1.9 Hz, 1H), 7.64-7.58 (m, 3H), 7.22 (s, 1H), 6.89 (s, 1H), 3.90 (s, 3H), 3.64 (q, J=6.8 Hz, 2H), 3.11 (t, J=7.3 Hz, 2H), 2.78 (t, J=7.3 Hz, 2H), 1.60 (s, 9H), 1.21 (t, J=7.0 Hz, 3H). FIG. 116 shows the nuclear magnetic resonance of Compound 8-41.

### Free Base of Compound 8-41

Batch 2(free base): 1H NMR (400 MHz, METHANOL-d4) δ=8.88 (d, J=2.0 Hz, 1H), 8.55 (d, J=2.0 Hz, 1H), 8.23 (t, J=1.9 Hz, 1H), 7.64-7.58 (m, 3H), 7.22 (s, 1H), 6.89 (s, 1H), 3.90 (s, 3H), 3.64 (q, J=6.8 Hz, 2H), 3.11 (t, J=7.3 Hz, 2H), 2.78 (t, J=7.3 Hz, 2H), 1.60 (s, 9H), 1.21 (t, J=7.0 Hz, 3H).

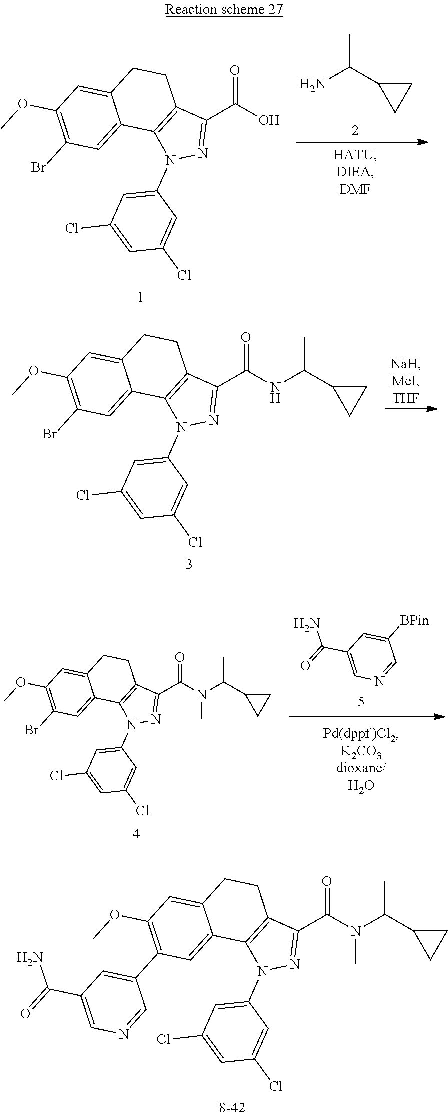

### Synthesis of Compound 8-42

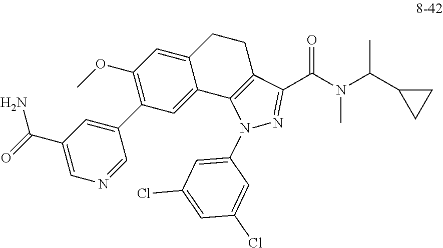 [SMILES: COc1cc2c(cc1-c1cncc(C(N)=O)c1)-c1c(c(C(=O)N(C)C(C)C3CC3)nn1-c1cc(Cl)cc(Cl)c1)CC2]

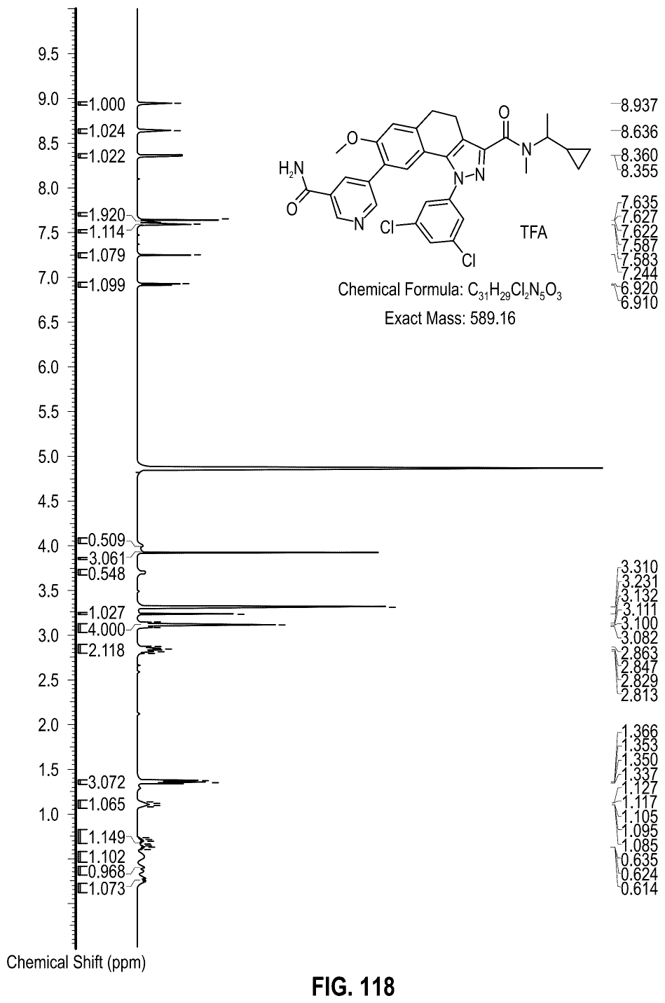

Compound 8-42 was synthesized according to a procedure similar to Reaction scheme 27. LCMS (ESI): m/z[M+H] calcd for C31H30Cl2N5O3 590.16; found: 590.3. 1H NMR (400 MHz, METHANOL-d4) δ=8.94 (s, 1H), 8.64 (s, 1H), 8.36 (br d, J=1.7 Hz, 1H), 7.65-7.61 (m, 2H), 7.58 (d, J=1.5 Hz, 1H), 7.24 (s, 1H), 6.91 (d, J=3.9 Hz, 1H), 4.02-3.95 (m, 0.5H), 3.93-3.91 (m, 3H), 3.73-3.66 (m, 0.5H), 3.23 (s, 1H), 3.14-3.07 (m, 4H), 2.88-2.79 (m, 2H), 1.35 (dd, J=5.1, 6.5 Hz, 3H), 1.15-1.07 (m, 1H), 0.74-0.59 (m, 1H), 0.58-0.46 (m, 1H), 0.44-0.33 (m, 1H), 0.30-0.20 (m, 1H). FIG. 118 shows the nuclear magnetic resonance of Compound 8-42.

### Synthesis of Compound 8-43

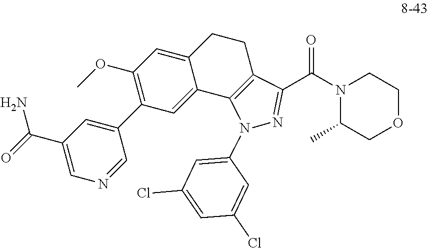 [SMILES: COc1cc2c(cc1-c1cncc(C(N)=O)c1)-c1c(c(C(=O)N3CCOC[C@@H]3C)nn1-c1cc(Cl)cc(Cl)c1)CC2]

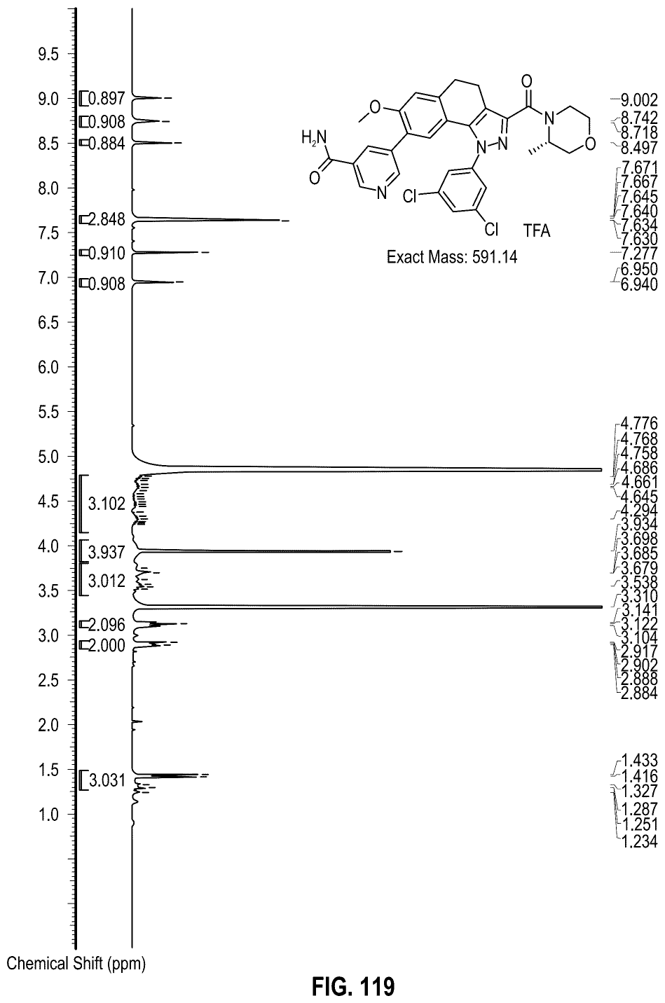

Compound 8-43 was synthesized according to a procedure similar to Reaction scheme 27. LCMS (ESI): m/z[M+H] calcd for C30H27N5O4Cl2: 592.14; found: 592.3. 1H NMR (400 MHz, METHANOL-d4) δ=9.00 (s, 1H), 8.79-8.67 (m, 1H), 8.50 (s, 1H), 7.68-7.60 (m, 3H), 7.28 (s, 1H), 6.97-6.89 (m, 1H), 4.79-4.15 (m, 3H), 3.93 (s, 4H), 3.79-3.44 (m, 3H), 3.16-3.09 (m, 2H), 2.91-2.85 (m, 1H), 1.46-1.27 (m, 3H). FIG. 119 shows the nuclear magnetic resonance of Compound 8-43.

### Synthesis of Compound 8-45

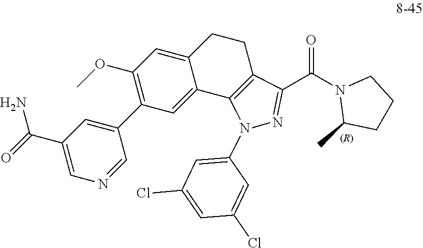

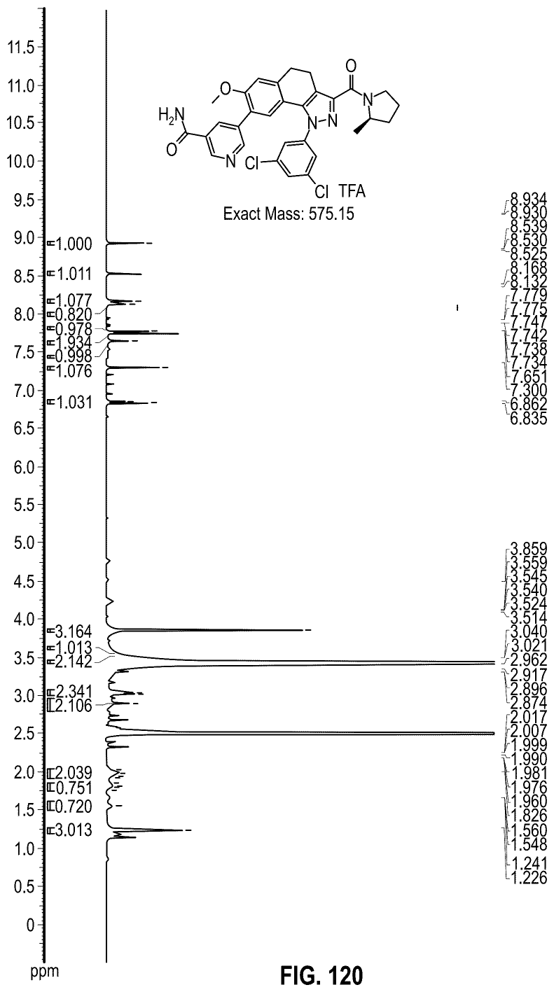

Compound 8-45 was synthesized according to a procedure similar to Reaction scheme 27. LCMS (ESI): m/z[M+H] calcd for C₃₀H₂₇N₅O₃Cl₂: 576.47; found: 576.3. ¹H NMR (400 MHz, DMSO-d6) δ=8.93 (d, J=1.6 Hz, 1H), 8.55-8.51 (m, 1H), 8.17 (d, J=2.0 Hz, 1H), 8.13 (s, 1H), 7.79-7.77 (m, 1H), 7.76-7.73 (m, 2H), 7.65 (s, 1H), 7.30 (s, 1H), 6.88-6.82 (m, 1H), 3.86 (s, 3H), 3.57-3.53 (m, 1H), 3.52 (m, 2H), 3.03 (d, J=7.6 Hz, 2H), 2.98-2.82 (m, 2H), 2.05-1.91 (m, 2H), 1.85-1.75 (m, 1H), 1.62-1.50 (m, 1H), 1.23 (d, J=6.0 Hz, 3H). FIG. 120 shows the nuclear magnetic resonance of Compound 8-45.

### Synthesis of Compound 8-46

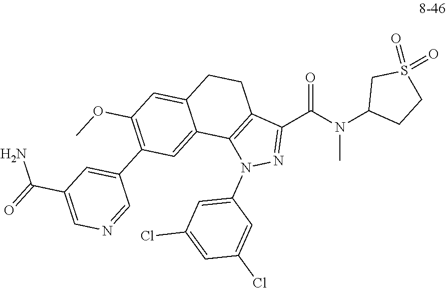 [SMILES: COc1cc2c(cc1-c1cncc(C(N)=O)c1)-c1c(c(C(=O)N(C)C3CCS(=O)(=O)C3)nn1-c1cc(Cl)cc(Cl)c1)CC2]

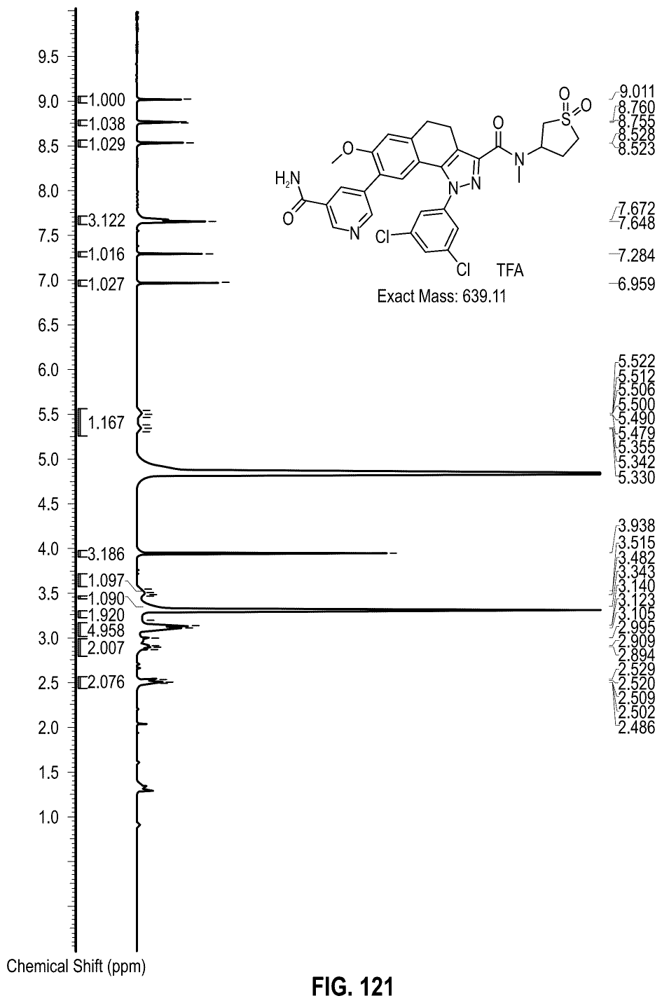

Compound 8-46 was synthesized according to a procedure similar to Reaction scheme 27. LCMS (ESI): m/z[M+H] calcd for C₃₀H₂₇N₅O₅Cl₂S:640.11; found: 640.3. 1H NMR (400 MHz, METHANOL-d4) δ=9.01 (d, J=1.6 Hz, 1H), 8.76 (d, J=2.0 Hz, 1H), 8.52 (t, J=2.0 Hz, 1H), 7.71-7.62 (m, 3H), 7.28 (s, 1H), 6.96 (s, 1H), 5.56-5.25 (m, 1H), 3.94 (s, 3H), 3.59-3.46 (m, 1H), 3.34 (s, 1H), 3.29-3.21 (m, 2H), 3.17-3.01 (m, 5H), 2.98-2.79 (m, 2H), 2.56-2.43 (m, 2H). FIG. 121 shows the nuclear magnetic resonance of Compound 8-46.

### Synthesis of Compound 8-47

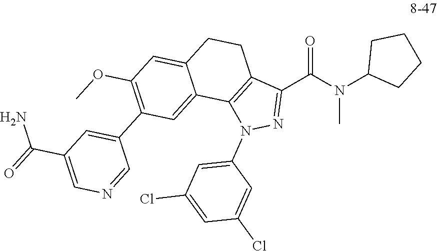 [SMILES: COc1cc2c(cc1-c1cncc(C(N)=O)c1)-c1c(c(C(=O)N(C)C3CCCC3)nn1-c1cc(Cl)cc(Cl)c1)CC2]

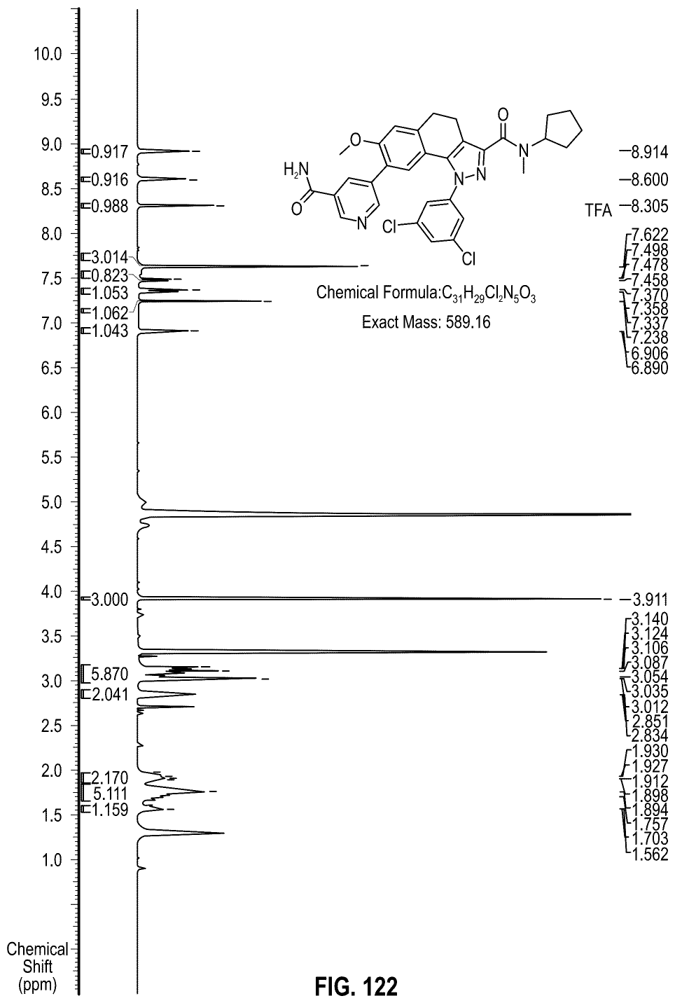

Compound 8-47 was synthesized according to a procedure similar to Reaction scheme 27. LCMS (ESI): m/z[M+H] calcd for C33H31N5O5Cl2F3: 590.16; found: 590.1. 1H NMR (400 MHz, METHANOL-d4) δ=8.91 (s, 1H), 8.60 (br s, 1H), 8.30 (s, 1H), 7.62 (s, 3H), 7.51-7.44 (m, 1H), 7.38-7.32 (m, 1H), 7.24 (s, 1H), 6.93-6.87 (m, 1H), 3.91 (s, 3H), 3.17-2.98 (m, 6H), 2.84 (br d, J=6.8 Hz, 2H), 1.97-1.85 (m, 2H), 1.84-1.65 (m, 5H), 1.56 (br s, 1H). FIG. 122 shows the nuclear magnetic resonance of Compound 8-47.

### Synthesis of Compound 8-49

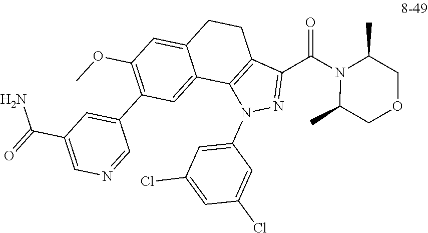 [SMILES: COc1cc2c(cc1-c1cncc(C(N)=O)c1)-c1c(c(C(=O)N3[C@H](C)COC[C@@H]3C)nn1-c1cc(Cl)cc(Cl)c1)CC2]

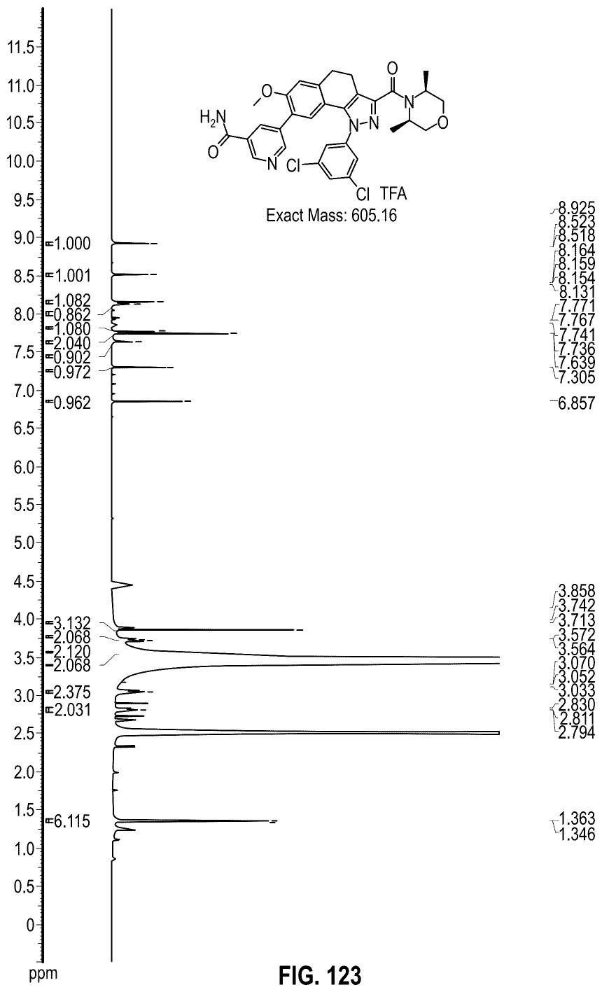

Compound 8-49 was synthesized according to a procedure similar to Reaction scheme 27. LCMS (ESI): m/z[M+H] calcd for C₃₁H₂₉N₅O₄Cl₂: 606.50; found:606.3. ¹H NMR (400 MHz, DMSO-d6) δ=8.93 (d, J=2.0 Hz, 1H), 8.52 (d, J=2.0 Hz, 1H), 8.16 (t, J=2.0 Hz, 1H), 8.13 (s, 1H), 7.78-7.76 (m, 1H), 7.74 (d, J=1.9 Hz, 2H), 7.64 (s, 1H), 7.30 (s, 1H), 6.86 (s, 1H), 3.86 (s, 3H), 3.73 (d, J=11.6 Hz, 2H), 3.57 (d, J=3.2 Hz, 2H), 3.55-3.54 (m, 2H), 3.05 (t, J=7.2 Hz, 2H), 2.84-2.78 (m, 2H), 1.35 (d, J=6.8 Hz, 6H). FIG. 123 shows the nuclear magnetic resonance of Compound 8-49.

### Synthesis of Compound 8-50

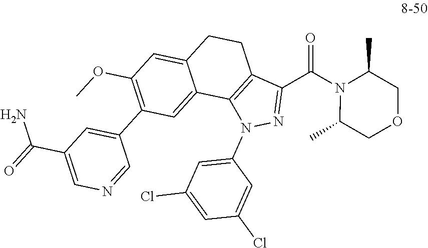 [SMILES: COc1cc2c(cc1-c1cncc(C(N)=O)c1)-c1c(c(C(=O)N3[C@@H](C)COC[C@@H]3C)nn1-c1cc(Cl)cc(Cl)c1)CC2]

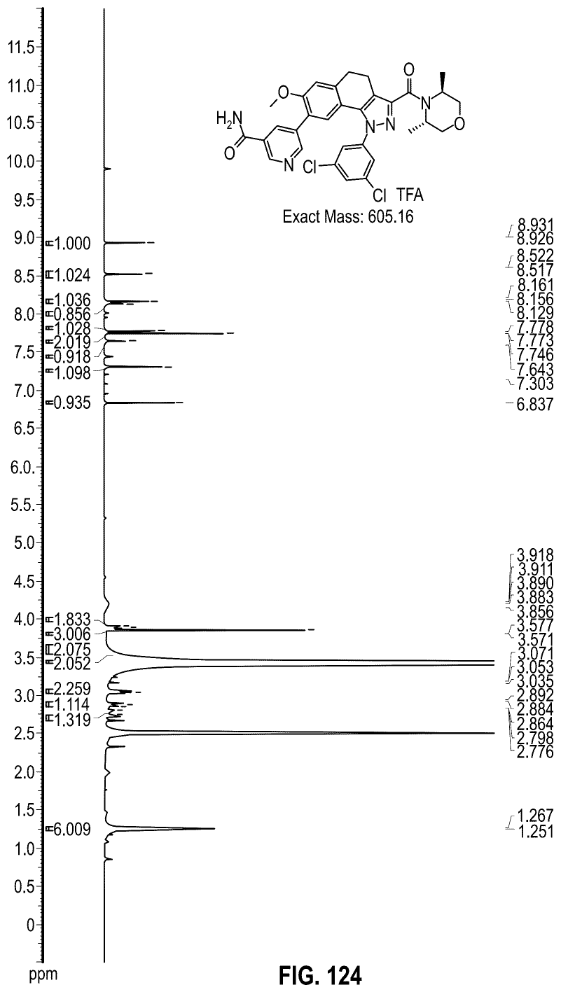

Compound 8-50 was synthesized according to a procedure similar to Reaction scheme 27. LCMS (ESI): m/z[M+H] calcd for C₃₁H₂₉N₅O₄Cl₂: 606.50; found:606.2. ¹H NMR (400 MHz, DMSO-d6) δ=8.93 (d, J=2.0 Hz, 1H), 8.52 (d, J=2.0 Hz, 1H), 8.16 (t, J=2.0 Hz, 1H), 8.13 (s, 1H), 7.79-7.76 (m, 1H), 7.74 (d, J=2.0 Hz, 2H), 7.64 (s, 1H), 7.30 (s, 1H), 6.84 (s, 1H), 3.90 (m, 2H), 3.86 (s, 3H), 3.59 (m, 2H), 3.54-3.51 (m, 2H), 3.08-3.02 (m, 2H), 2.92-2.86 (m, 1H), 2.82-2.74 (m, 1H), 1.26 (d, J=6.4 Hz, 6H). FIG. 124 shows the nuclear magnetic resonance of Compound 8-50.

### Synthesis of Compound 8-52A

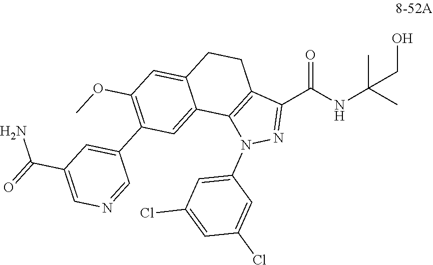 [SMILES: COc1cc2c(cc1-c1cncc(C(N)=O)c1)-c1c(c(C(=O)NC(C)(C)CO)nn1-c1cc(Cl)cc(Cl)c1)CC2]

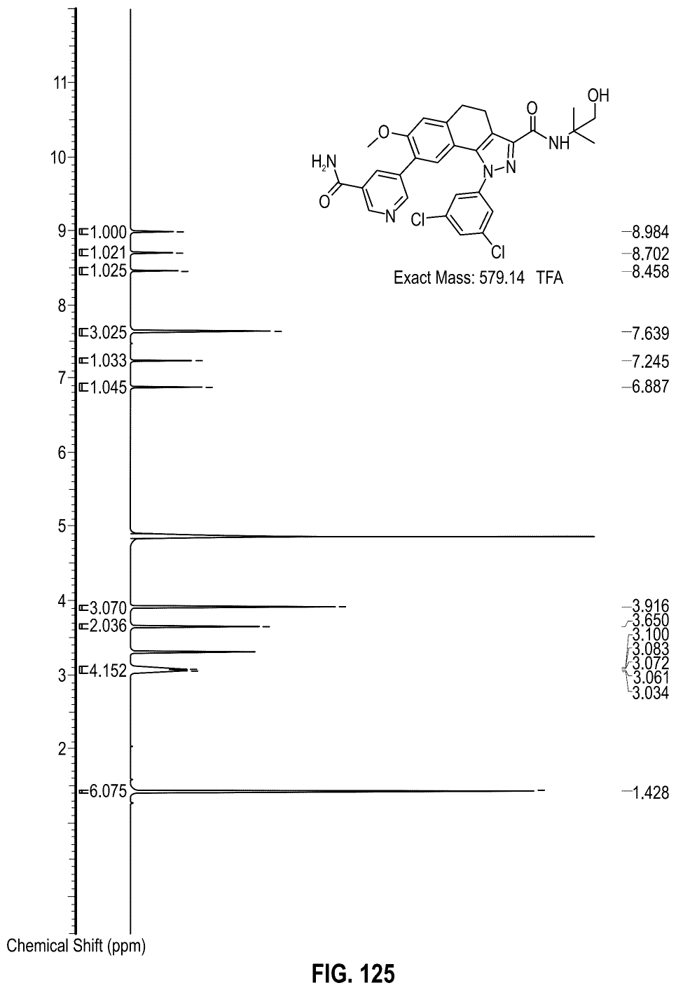

Compound 8-52A was synthesized according to a procedure similar to Reaction scheme 27. LCMS (ESI): m/z[M+H] calcd for C31H28N5O6Cl2: 580.14; found: 580.2. 1H NMR (400 MHz, METHANOL-d4) δ=8.98 (s, 1H), 8.70 (s, 1H), 8.46 (s, 1H), 7.64 (s, 3H), 7.24 (s, 1H), 6.89 (s, 1H), 3.92 (s, 3H), 3.65 (s, 2H), 3.12-3.01 (in 4H), 1.43 (s, 6H). FIG. 125 shows the nuclear magnetic resonance of Compound 8-52A.

### Synthesis of Compound 8-54A

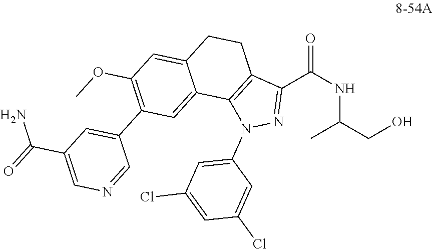 [SMILES: COc1cc2c(cc1-c1cncc(C(N)=O)c1)-c1c(c(C(=O)NC(C)CO)nn1-c1cc(Cl)cc(Cl)c1)CC2]

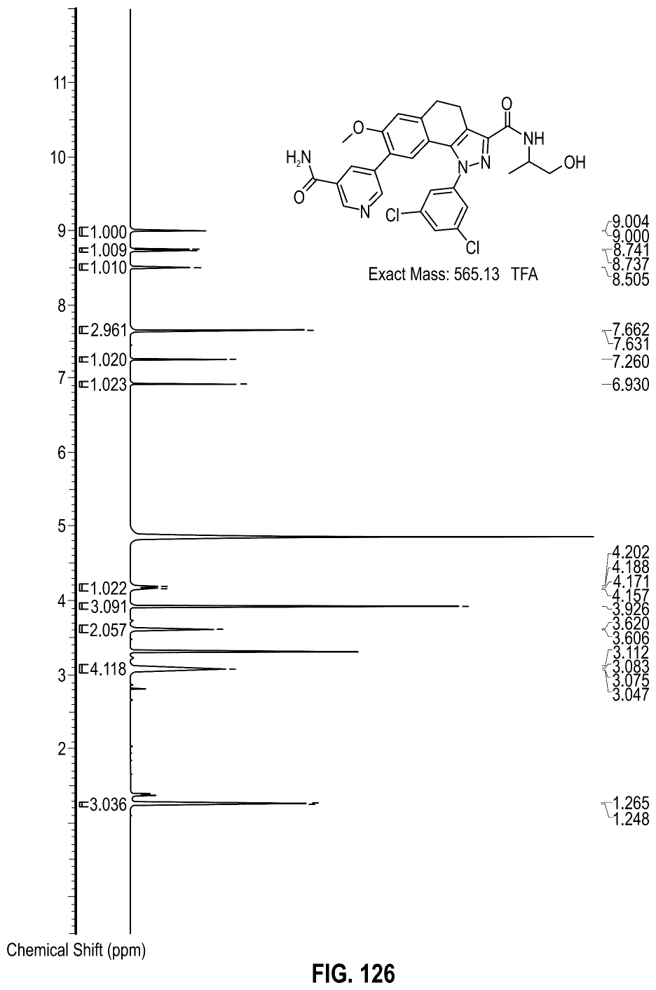

Compound 8-54A was synthesized according to a procedure similar to Reaction scheme 27. LCMS (ESI): m z[M+H] calcd for C₃₀H₂₆N₅O₆Cl₂:566.13; found: 566.1. H NMR (400 MHz, METHANOL-d4) δ=9.00 (d, J=1.3 Hz, 1H), 8.74 (d, J=1.6 Hz, 1H), 8.51 (s, 1H), 7.70-7.61 (m, 3H), 7.26 (s, 1H), 6.93 (s, 1H), 4.24-4.13 (i, 1H), 3.93 (s, 3H), 3.61 (d, J=5.4 Hz, 2H), 3.14-3.02 (m, 4H), 1.26 (d, J=6.7 Hz, 3H). FIG. 126 shows the nuclear magnetic resonance of Compound 8-54A.

### Synthesis of Compound 8-55

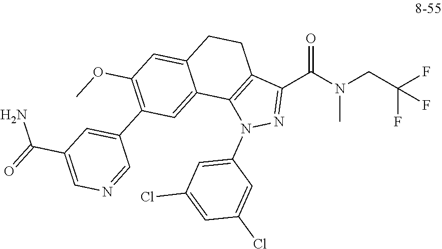 [SMILES: COc1cc2c(cc1-c1cncc(C(N)=O)c1)-c1c(c(C(=O)N(C)CC(F)(F)F)nn1-c1cc(Cl)cc(Cl)c1)CC2]

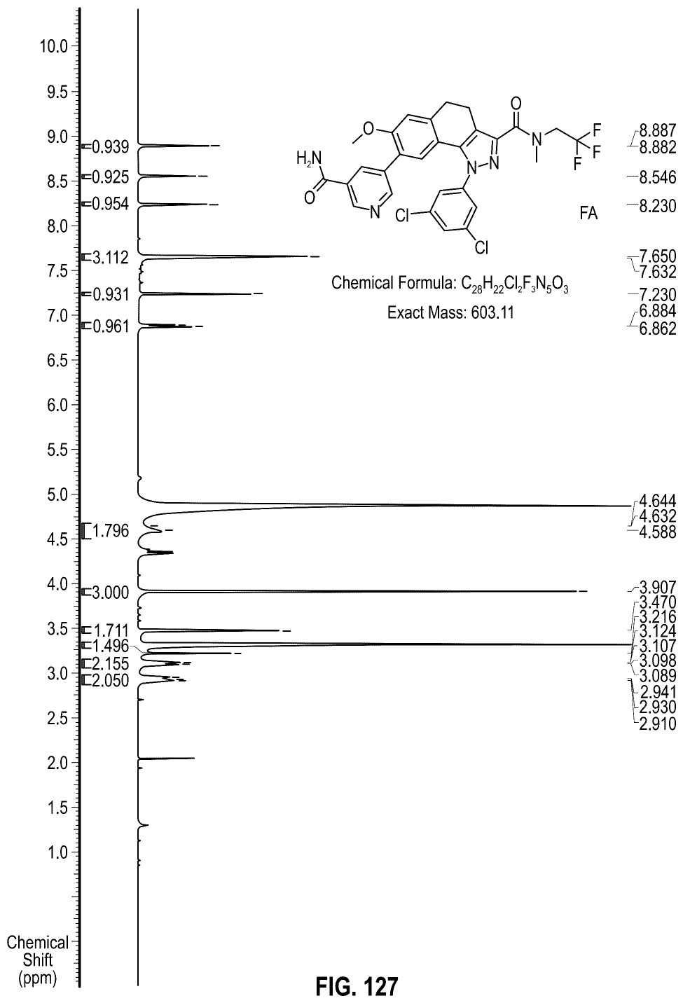

Compound 8-55 was synthesized according to a procedure similar to Reaction scheme 27. LCMS (ESI): m/z[M+H] calcd for C₂₈H₂₃N₅O₃Cl₂F₃: 604.11; found: 604.0. ¹H NMR (400 MHz, DMSO-d4) δ=9.89 (s, 1H), 8.90 (d, J=1.9 Hz, 1H), 8.48 (br s, 1H), 8.16-8.09 (m, 2H), 7.99-7.92 (m, 1H), 7.80-7.72 (m, 2H), 7.69 (s, 1H), 7.62 (s, 1H), 7.46-7.39 (m, 1H), 7.31-7.25 (m, 2H), 6.85-6.77 (m, 1H), 4.86-4.78 (m, 1H), 4.43-4.34 (m, 2H), 3.84 (s, 3H), 3.10 (br s, 1H), 3.07-2.99 (m, 3H), 2.91 (br d, J=5.3 Hz, 1H), 2.88 (s, 1H), 2.86-2.80 (m, 2H), 2.72 (s, 1H), 1.25-1.19 (m, 1H), 1.18-1.09 (m, 2H). FIG. 127 shows the nuclear magnetic resonance of Compound 8-55.

### Synthesis of Compound 8-56

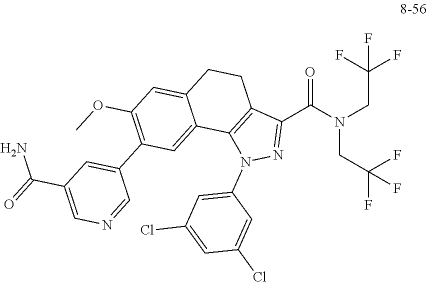 [SMILES: COc1cc2c(cc1-c1cncc(C(N)=O)c1)-c1c(c(C(=O)N(CC(F)(F)F)CC(F)(F)F)nn1-c1cc(Cl)cc(Cl)c1)CC2]

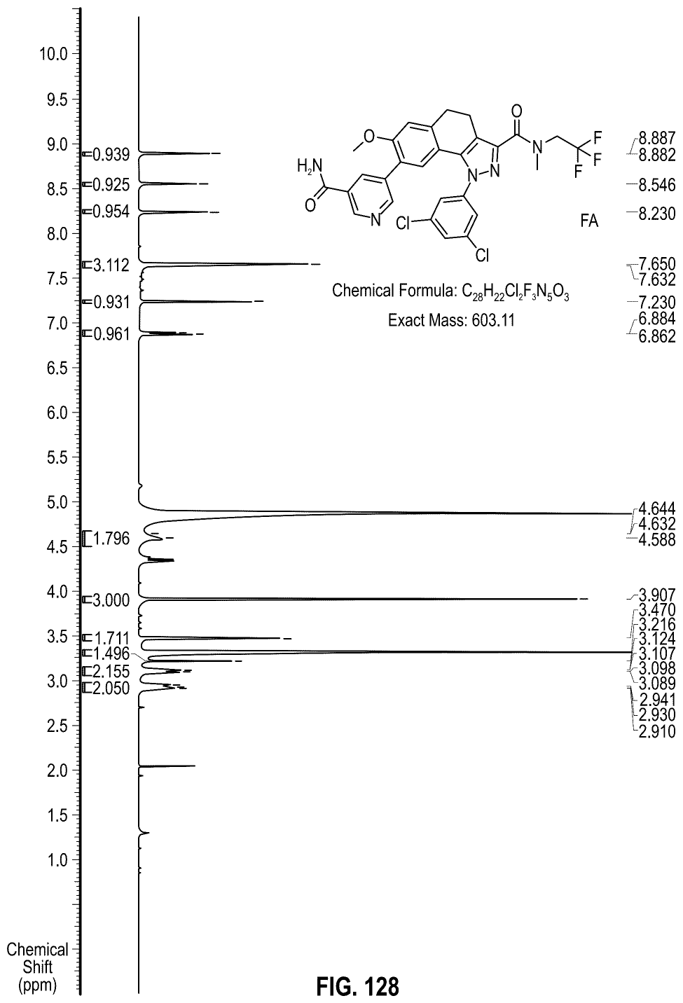

Compound 8-56 was synthesized according to a procedure similar to Reaction scheme 27. LCMS (ESI): m/z[M+H] calcd for C₃₀H₂₄N₅O₅Cl₂F₆: 672.09; found: 672.0. ¹H NMR (400 MHz, METHANOL-d₄) δ=8.89 (d, J=1.9 Hz, 1H), 8.55 (d, J=1.5 Hz, 1H), 8.24 (t, J=1.6 Hz, 1H), 7.68-7.63 (m, 3H), 7.23 (s, 1H), 6.87 (s, 1H), 5.10-4.97 (m, 2H), 4.52-4.37 (m, 2H), 3.91 (s, 3H), 3.13-3.08 (m, 2H), 3.01-2.94 (m, 2H). FIG. 128 shows the nuclear magnetic resonance of Compound 8-56.

### Synthesis of Compound 8-62

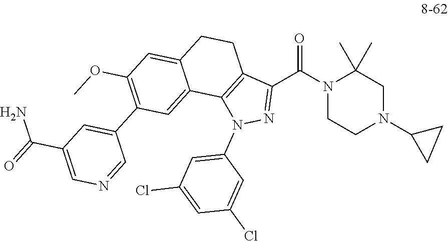 [SMILES: COc1cc2c(cc1-c1cncc(C(N)=O)c1)-c1c(c(C(=O)N3CCN(C4CC4)CC3(C)C)nn1-c1cc(Cl)cc(Cl)c1)CC2]

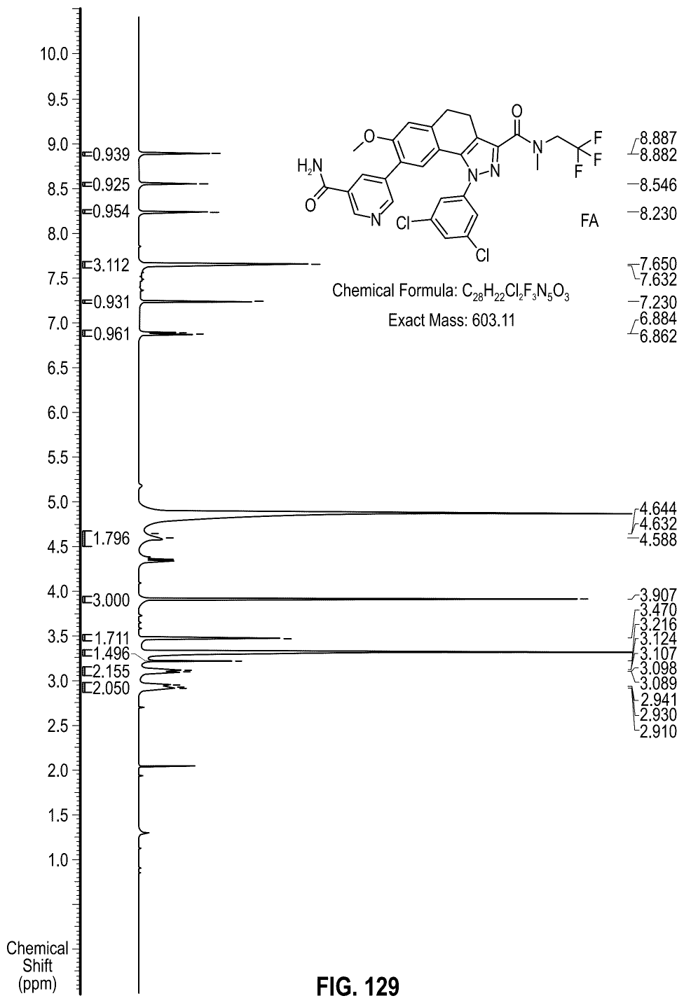

Compound 8-62 was synthesized according to a procedure similar to Reaction scheme 27. LCMS (ESI): m/z[M+H] calcd for C₃₄H₃₄Cl₂N₆O₃: 644.21; found: 645.3. ¹H NMR: ((400 MHz, METHANOL-d₄) δ=8.90 (d, J=1.6 Hz, 1H), 8.56 (s, 1H), 8.26 (s, 1H), 7.67 (d, J=1.6 Hz, 1H), 7.64 (d, J=1.6 Hz, 2H), 7.23 (s, 1H), 6.85 (s, 1H), 4.32-3.94 (m, 2H), 3.91 (s, 3H), 3.59 (br s, 2H), 3.47 (s, 2H), 3.14-3.07 (m, 2H), 2.96-2.87 (m, 3H), 1.69 (s, 6H), 1.07 (br s, 2H), 0.98 (br d, J=7.0 Hz, 2H). FIG. 129 shows the nuclear magnetic resonance of Compound 8-62.

### Synthesis of Compound 8-64

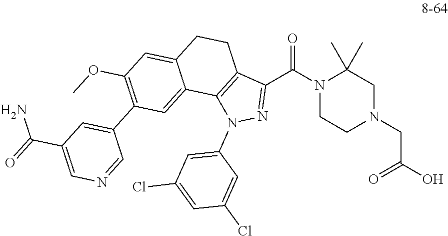 [SMILES: COc1cc2c(cc1-c1cncc(C(N)=O)c1)-c1c(c(C(=O)N3CCN(CC(=O)O)CC3(C)C)nn1-c1cc(Cl)cc(Cl)c1)CC2]

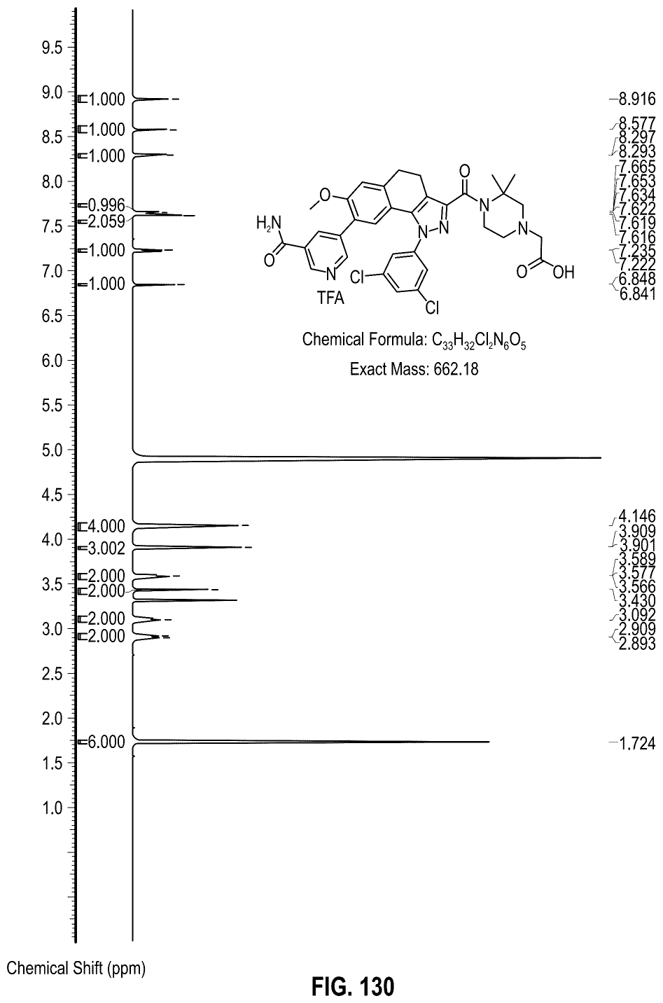

Compound 8-64 was synthesized according to a procedure similar to Reaction scheme 27. LCMS (ESI): m/z[M+H] calcd for C₃₃H₃₂Cl₂N₆O₅:662.18; found:663.2. ¹H NMR: (400 MHz, METHANOL-d₄) δ=8.92 (s, 1H), 8.58 (s, 1H), 8.29 (br d, J=1.6 Hz, 1H), 7.68-7.64 (m, 1H), 7.64-7.60 (m, 2H), 7.23 (br d, J=4.9 Hz, 1H), 6.84 (d, J=2.8 Hz, 1H), 4.15 (br s, 4H), 3.90 (d, J=3.3 Hz, 3H), 3.58 (br t, J=4.8 Hz, 2H), 3.43 (s, 2H), 3.09 (br s, 2H), 2.90 (br d, J=6.4 Hz, 2H), 1.72 (s, 6H). FIG. 130 shows the nuclear magnetic resonance of Compound 8-64.

### Synthesis of Compound 8-65

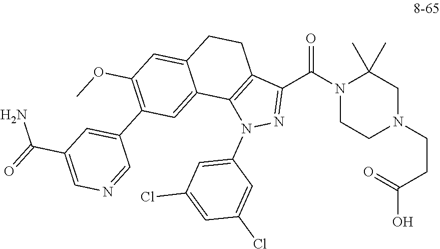 [SMILES: COc1cc2c(cc1-c1cncc(C(N)=O)c1)-c1c(c(C(=O)N3CCN(CCC(=O)O)CC3(C)C)nn1-c1cc(Cl)cc(Cl)c1)CC2]

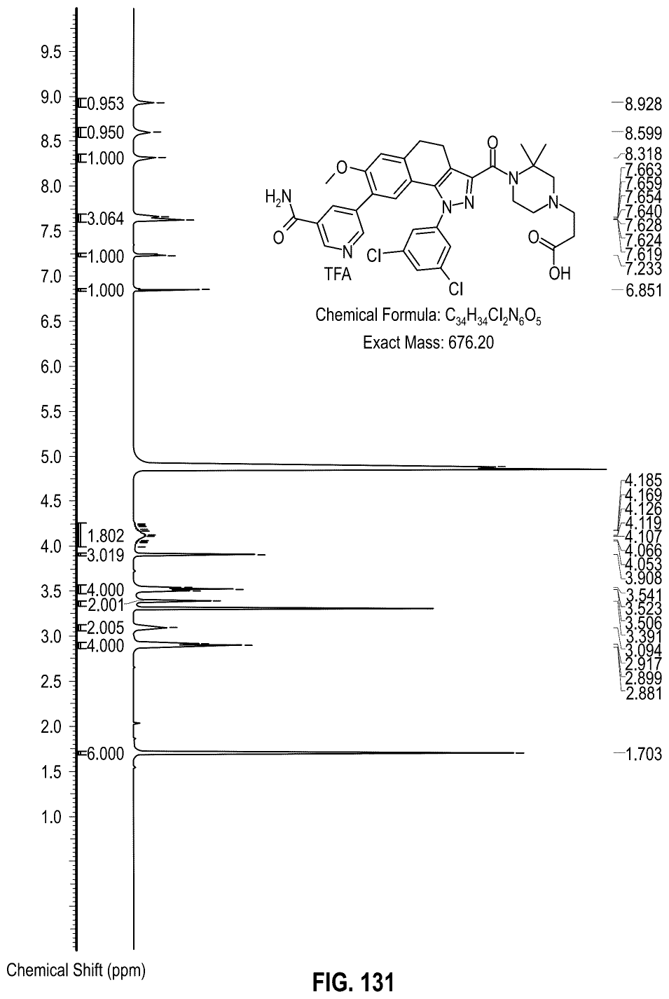

Compound 8-65 was synthesized according to a procedure similar to Reaction scheme 27. LCMS (ESI): m/z[M+H] calcd for C₃₄H₃₄Cl₂N₆O₃: 676.20; found: 677.2. ¹H NMR: (400 MHz, METHANOL-d₄) δ=8.93 (br s, 1H), 8.60 (br s, 1H), 8.32 (br s, 1H), 7.69-7.60 (m, 3H), 7.23 (br s, 1H), 6.85 (s, 1H), 4.25-3.99 (m, 2H), 3.91 (br s, 3H), 3.52 (br t, J=7.1 Hz, 4H), 3.39 (s, 2H), 3.09 (br s, 2H), 2.93-2.86 (m, 4H), 1.70 (s, 6H). FIG. 131 shows the nuclear magnetic resonance of Compound 8-65.

### Synthesis of Compound 8-69

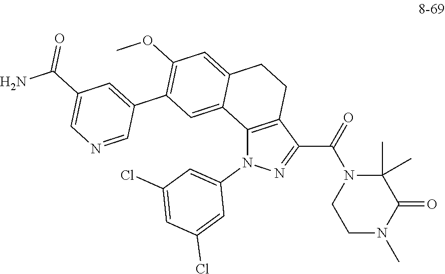 [SMILES: COc1cc2c(cc1-c1cncc(C(N)=O)c1)-c1c(c(C(=O)N3CCN(C)C(=O)C3(C)C)nn1-c1cc(Cl)cc(Cl)c1)CC2]

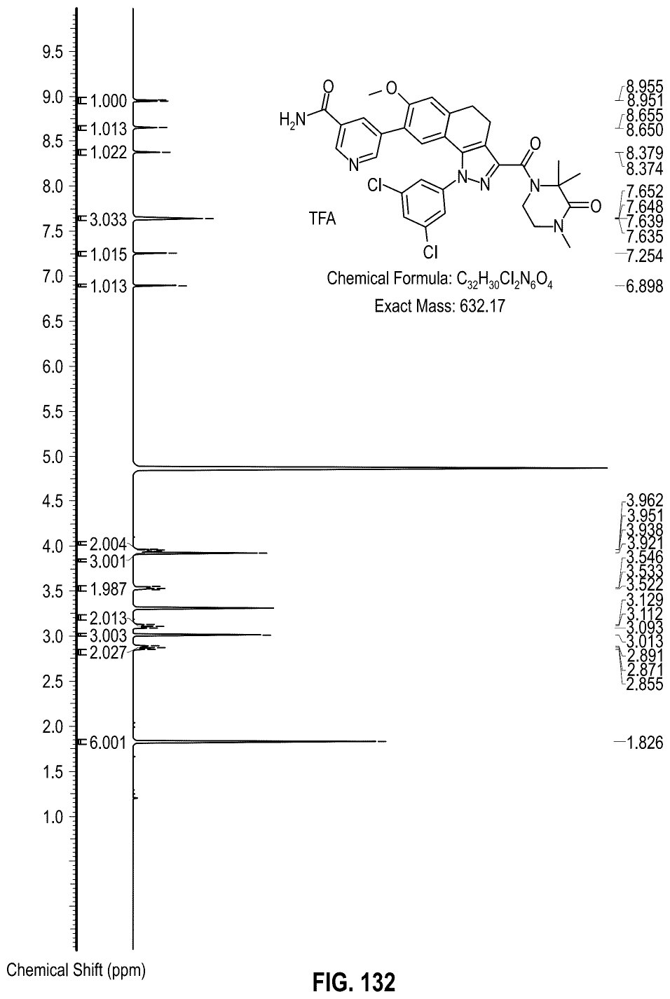

Compound 8-69 was synthesized according to a procedure similar to Reaction scheme 27. LCMS (ESI): m z[M+H] calcd for C₃₂H₃₁Cl₂N₆O₄: 633.17; found: 633.2. ¹H NMR (400 MHz, METHANOL-d4) δ=8.95 (d, J=1.8 Hz, 1H), 8.65 (d, J=2.0 Hz, 1H), 8.41-8.35 (m, 1H), 7.67-7.61 (m, 3H), 7.25 (s, 1H), 6.90 (s, 1H), 3.98-3.93 (m, 2H), 3.92 (s, 3H), 3.56-3.51 (m, 2H), 3.14-3.08 (m, 2H), 3.01 (s, 3H), 2.90-2.84 (m, 2H), 1.83 (s, 6H). FIG. 132 shows the nuclear magnetic resonance of Compound 8-69.

### Synthesis of Compound 8-70

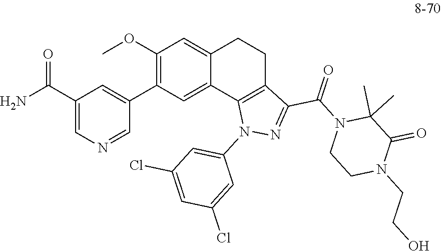 [SMILES: COc1cc2c(cc1-c1cncc(C(N)=O)c1)-c1c(c(C(=O)N3CCN(CCO)C(=O)C3(C)C)nn1-c1cc(Cl)cc(Cl)c1)CC2]

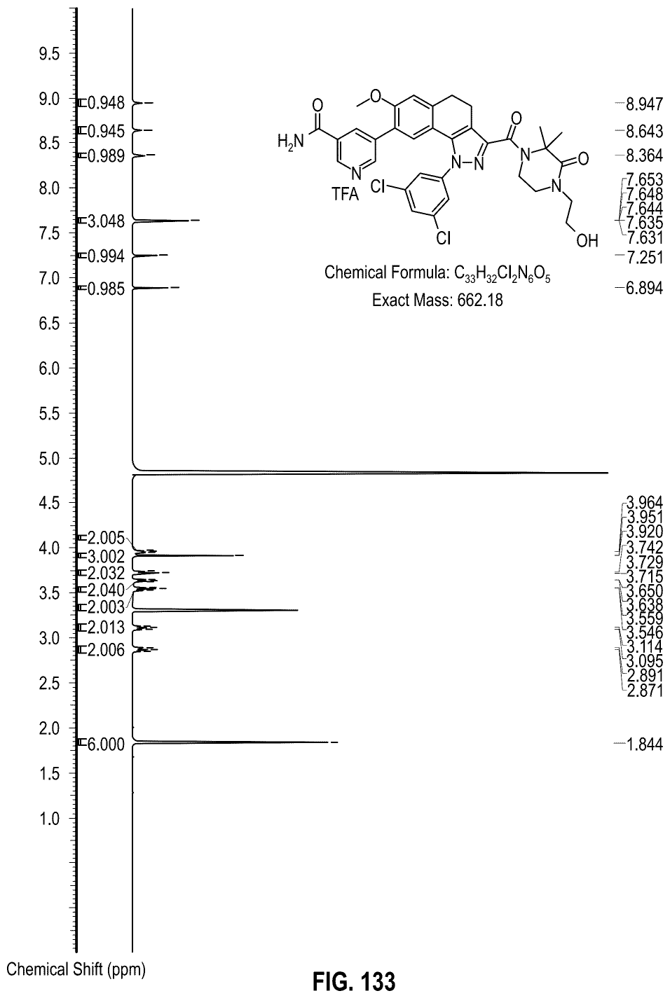

Compound 8-70 was synthesized according to a procedure similar to Reaction scheme 27. LCMS (ESI): m/z[M+H] calcd for C₃₃H₃₂Cl₂N₆O₅: 663.18; found: 663.3. ¹H NMR (400 MHz, METHANOL-d₄) δ=8.95 (s, 1H), 8.64 (br s, 1H), 8.36 (d, J=1.6 Hz, 1H), 7.67-7.61 (m, 3H), 7.25 (s, 1H), 6.89 (s, 1H), 3.99-3.94 (m, 2H), 3.92 (s, 3H), 3.76-3.70 (m, 2H), 3.67-3.61 (m, 2H), 3.58-3.51 (m, 2H), 3.11 (br t, J=7.4 Hz, 2H), 2.91-2.83 (m, 2H), 1.84 (s, 6H). FIG. 133 shows the nuclear magnetic resonance of Compound 8-70.

### Synthesis of Compound 8-71

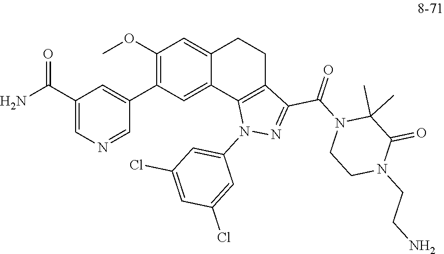 [SMILES: COc1cc2c(cc1-c1cncc(C(N)=O)c1)-c1c(c(C(=O)N3CCN(CCN)C(=O)C3(C)C)nn1-c1cc(Cl)cc(Cl)c1)CC2]

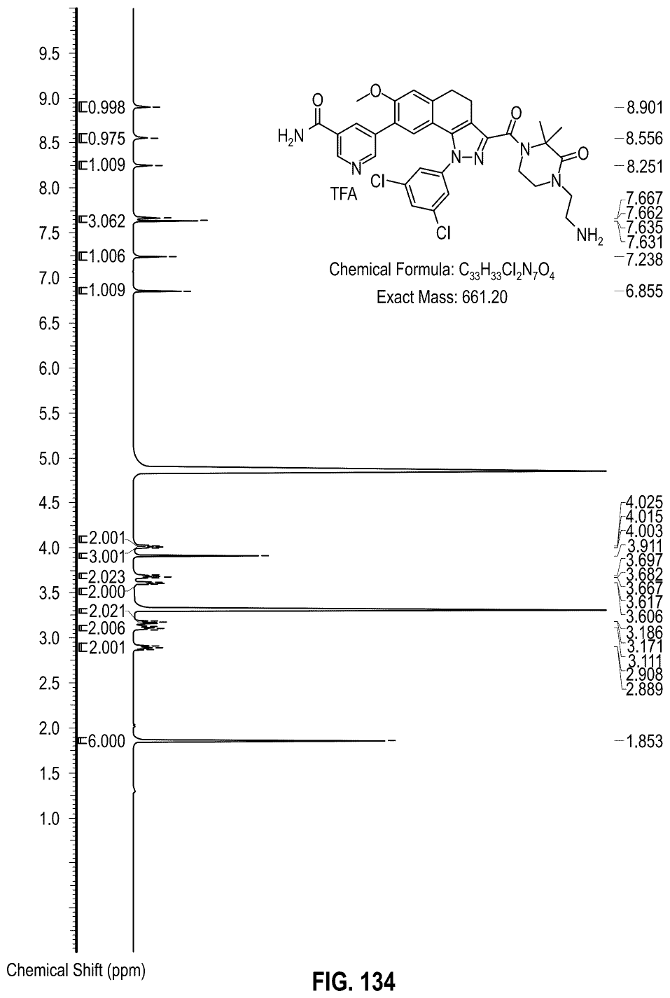

Compound 8-71 was synthesized according to a procedure similar to Reaction scheme 27. LCMS (ESI):m/z[M+H] calcd for C₃₃H₃₄Cl₂N₇O₄: 662.20; found: 662.2. ¹H NMR (400 MHz, METHANOL-d₄) δ=8.90 (s, 1H), 8.56 (br s, 1H), 8.25 (br d, J=1.6 Hz, 1H), 7.68-7.62 (m, 3H), 7.24 (s, 1H), 6.86 (s, 1H), 4.05-3.98 (m, 2H), 3.91 (s, 3H), 3.68 (t, J=6.0 Hz, 2H), 3.64-3.58 (m, 2H), 3.17 (br t, J=6.0 Hz, 2H), 3.11 (br t, J=7.3 Hz, 2H), 2.94-2.85 (m, 2H), 1.85 (s, 6H). FIG. 134 shows the nuclear magnetic resonance of Compound 8-71.

### Synthesis of Compound 8-79

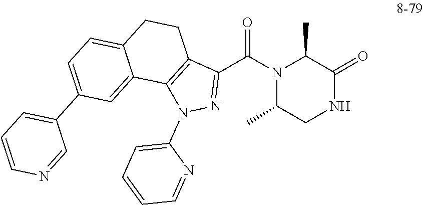 [SMILES: C[C@H]1CNC(=O)[C@H](C)N1C(=O)c1nn(-c2ccccn2)c2c1CCc1ccc(-c3cccnc3)cc1-2]

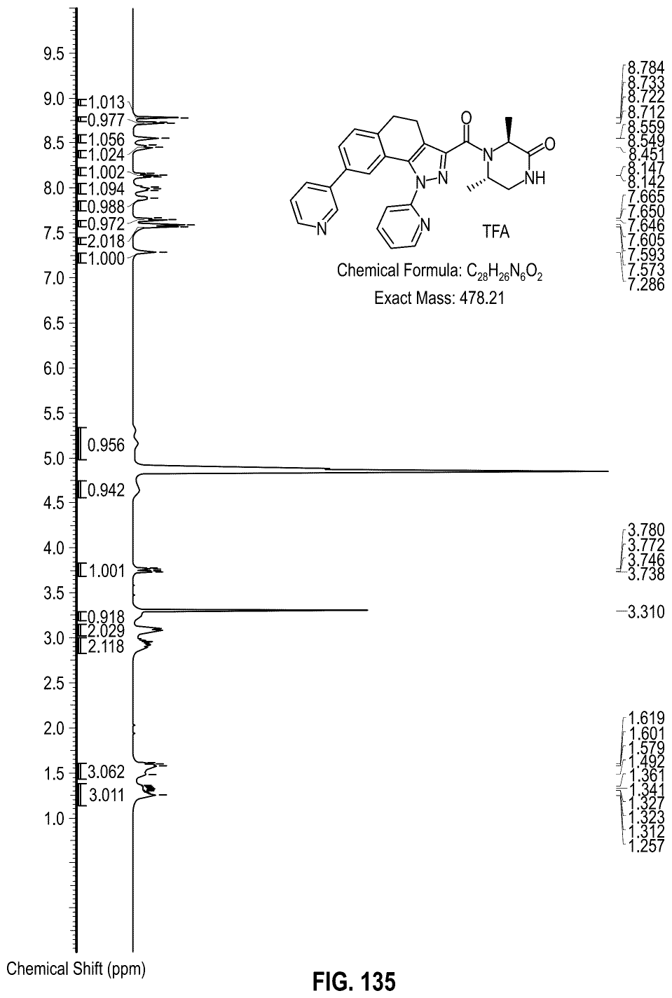

Compound 8-79 was synthesized according to a procedure similar to Reaction scheme 27. LCMS (ESI): m/z[M+H] calcd for C₂₈H₂₇N₆O₂: 479.21; found: 479.3. ¹H NMR (400 MHz, METHANOL-d4) δ=8.78 (br s, 1H), 8.72 (br t, J=4.1 Hz, 1H), 8.55 (br d, J=3.9 Hz, 1H), 8.49-8.41 (m, 1H), 8.19-8.09 (m, 1H), 8.05-7.93 (m, 1H), 7.89 (br d, J=2.9 Hz, 1H), 7.67-7.62 (m, 1H), 7.61-7.53 (m, 2H), 7.29 (br s, 1H), 5.33-5.11 (m, 1H), 4.75-4.56 (m, 1H), 3.76 (br dd, J=3.1, 13.8 Hz, 1H), 3.30-3.20 (m, 1H), 3.16-3.03 (m, 2H), 3.01-2.83 (m, 2H), 1.64-1.44 (m, 3H), 1.39-1.20 (m, 3H). FIG. 135 shows the nuclear magnetic resonance of Compound 8-79.

### Synthesis of Compound 8-82

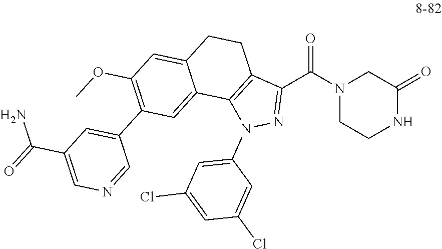 [SMILES: COc1cc2c(cc1-c1cncc(C(N)=O)c1)-c1c(c(C(=O)N3CCNC(=O)C3)nn1-c1cc(Cl)cc(Cl)c1)CC2]

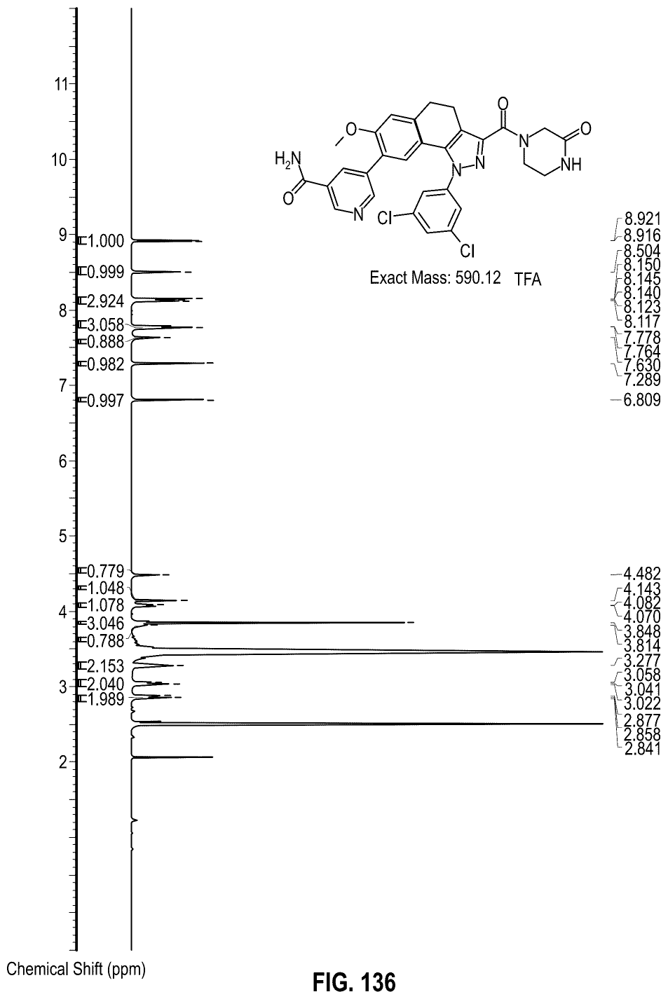

Compound 8-82 was synthesized according to a procedure similar to Reaction scheme 27. LCMS (ESI): m/z[M+H] calcd for C₂₉H₂₄N₆O₄Cl₂: 591.44; found: 591.1. ¹H NMR (400 MHz, DMSO-d6) δ=8.92 (d, J=2.0 Hz, 1H), 8.51 (d, J=1.9 Hz, 1H), 8.17-8.08 (m, 3H), 7.81-7.71 (m, 3H), 7.63 (s, 1H), 7.29 (s, 1H), 6.81 (s, 1H), 4.48 (s, 1H), 4.14 (s, 1H), 4.08 (br d, J=4.6 Hz, 1H), 3.85 (s, 3H), 3.82 (br s, 1H), 3.28 (br s, 2H), 3.09-2.99 (m, 2H), 2.91-2.82 (m, 2H). FIG. 136 shows the nuclear magnetic resonance of Compound 8-82.

### Synthesis of Compound 8-83

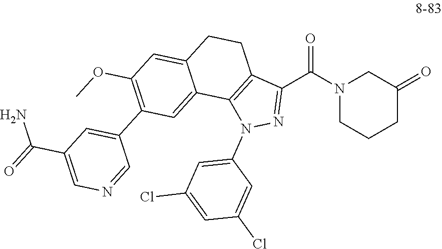 [SMILES: COc1cc2c(cc1-c1cncc(C(N)=O)c1)-c1c(c(C(=O)N3CCCC(=O)C3)nn1-c1cc(Cl)cc(Cl)c1)CC2]

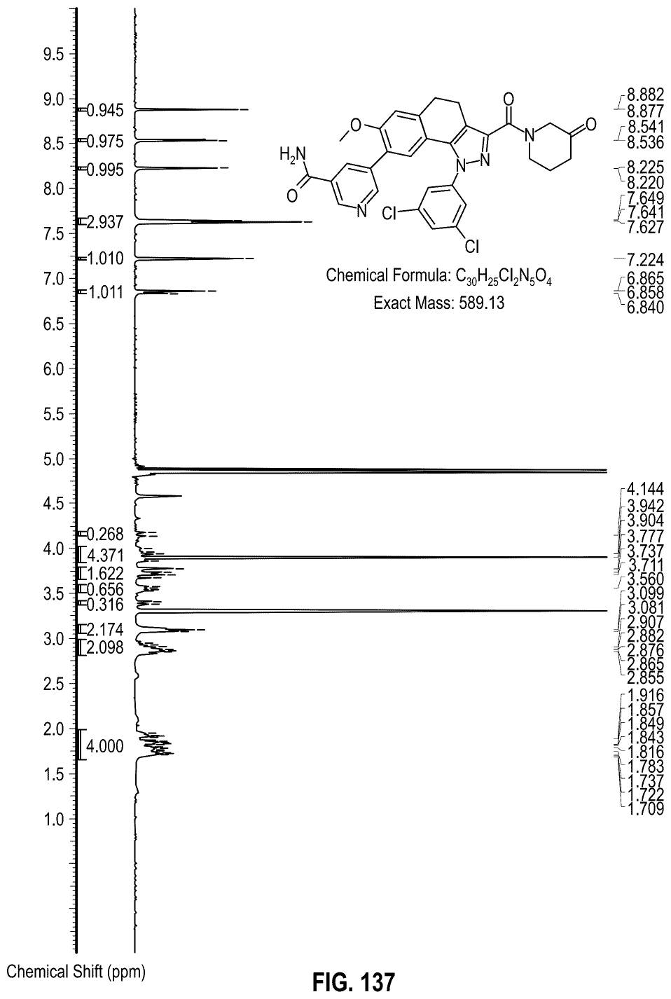

Compound 8-83 was synthesized according to a procedure similar to Reaction scheme 27. LCMS (ESI): m/z[M+H] calcd for C₃₀H₂₅Cl₂N₅O₄: 589.13; found: 590.1. ¹H NMR: (400 MHz, METHANOL-d₄) δ=8.88 (d, J 2.0 Hz, 1H), 8.54 (d, J 2.0 Hz, 1H), 8.22 (d, J 1.8 Hz, 1H), 7.66-7.61 (m, 3H), 7.22 (s, 1H), 6.88-6.83 (m, 1H), 4.02-3.84 (m, 4H), 3.80-3.66 (m, 2H), 3.60-3.51 (m, 1H), 3.09 (br d, J 7.1 Hz, 2H), 2.99-2.81 (m, 2H), 1.99-1.65 (m, 4H). FIG. 137 shows the nuclear magnetic resonance of Compound 8-83.

### Synthesis of Compound 8-84

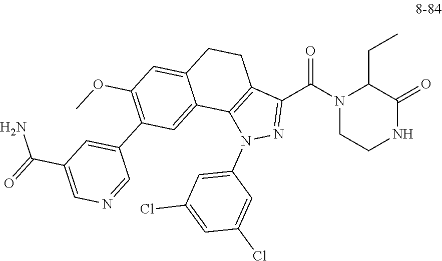 [SMILES: CCC1C(=O)NCCN1C(=O)c1nn(-c2cc(Cl)cc(Cl)c2)c2c1CCc1cc(OC)c(-c3cncc(C(N)=O)c3)cc1-2]

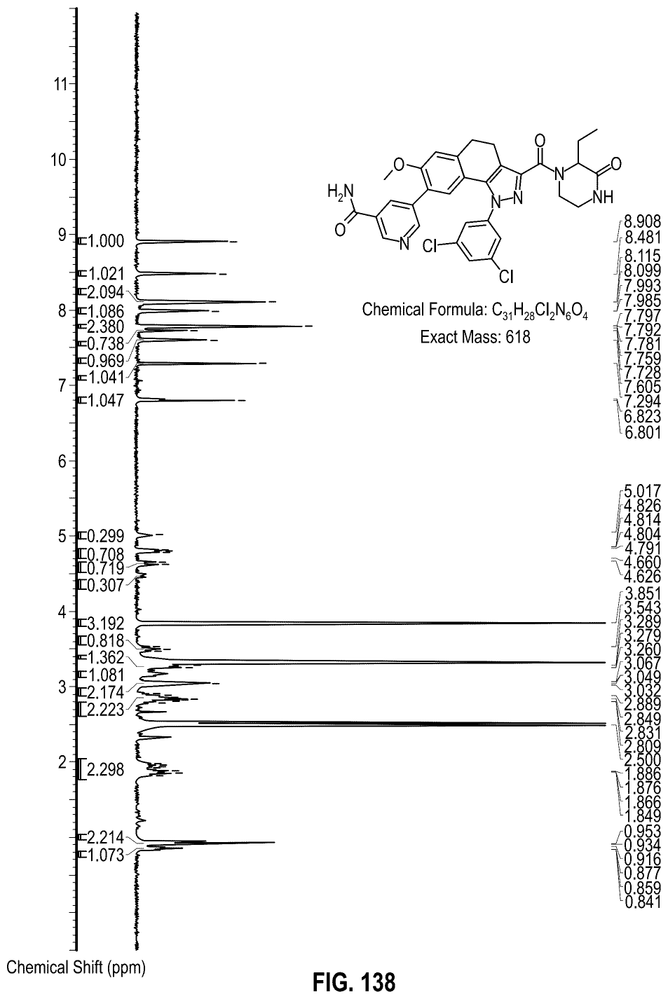

Compound 8-84 was synthesized according to a procedure similar to Reaction scheme 27. LCMS (ESI): m/z[M+H] calcd for C₃₁H₂₉O₄N₆Cl₂: 619; found: 619.2. 1H NMR (400 MHz, DMSO-d6) δ ppm 8.91 (s, 1H) 8.48 (s, 1H) 8.15-8.06 (m, 2H) 7.99 (br d, J=3.13 Hz, 1H) 7.81-7.77 (m, 2H) 7.76-7.70 (m, 1H) 7.60 (s, 1H) 7.29 (s, 1H) 6.85-6.76 (m, 1H) 5.02 (s, 1H) 4.81 (br dd, J=9.01, 5.13 Hz, 1H) 4.64 (br d, J=13.51 Hz, 1H) 4.69-4.58 (m, 1H) 4.54-4.41 (m, 1H) 3.85 (s, 3H) 3.57-3.44 (m, 1H) 3.29-3.24 (m, 1H) 3.21-3.12 (m, 1H) 3.10-2.99 (m, 2H) 2.95-2.76 (m, 2H) 2.05-1.77 (m, 2H) 0.93 (br t, J=7.38 Hz, 2H) 0.86 (br t, J=7.25 Hz, 1H). FIG. 138 shows the nuclear magnetic resonance of Compound 8-84.

### Synthesis of Compound 8-86

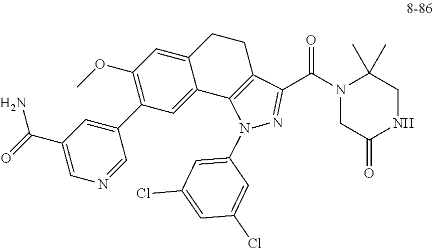 [SMILES: COc1cc2c(cc1-c1cncc(C(N)=O)c1)-c1c(c(C(=O)N3CC(=O)NCC3(C)C)nn1-c1cc(Cl)cc(Cl)c1)CC2]

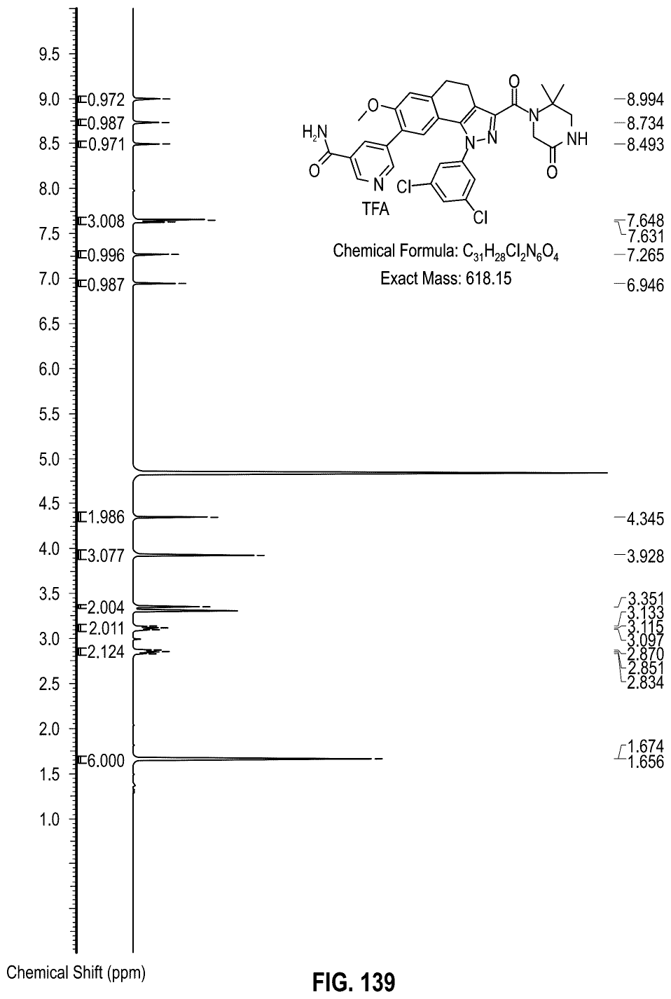

Compound 8-86 was synthesized according to a procedure similar to Reaction scheme 27. LCMS (ESI): m z[M+H] calcd for C₃₁H₂₉C₁₂N₆O₄: 619.15; found: 619.3. ¹H NMR (400 MHz, METHANOL-d₄) δ=8.99 (s, 1H), 8.73 (s, 1H), 8.49 (s, 1H), 7.68-7.60 (m, 3H), 7.26 (s, 1H), 6.95 (s, 1H), 4.34 (s, 2H), 3.93 (s, 3H), 3.35 (s, 2H), 3.11 (br t, J=7.3 Hz, 2H), 2.89-2.81 (m, 2H), 1.66 (s, 6H). FIG. 139 shows the nuclear magnetic resonance of Compound 8-86.

### Synthesis of Compound 8-87

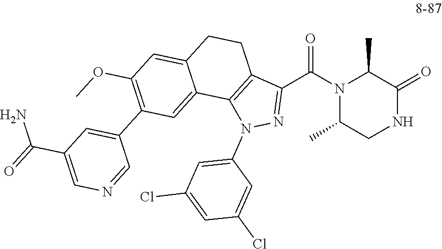 [SMILES: COc1cc2c(cc1-c1cncc(C(N)=O)c1)-c1c(c(C(=O)N3[C@@H](C)CNC(=O)[C@@H]3C)nn1-c1cc(Cl)cc(Cl)c1)CC2]

Compound 8-87 was synthesized according to a procedure similar to Reaction scheme 27. LCMS (ESI): m/z[M+H] calcd for C₃₁H₂₈Cl₂N₆O₄: 619.15; found: 619.3.¹H NMR (400 MHz, METHANOL-d4) δ=9.03 (s, 1H), 8.79 (s, 1H), 8.56 (br s, 1H), 7.69-7.62 (m, 3H), 7.29 (s, 1H), 6.98 (s, 1H), 5.30-5.07 (m, 1H), 4.75-4.50 (m, 1H), 3.94 (s, 3H), 3.75 (dd, J=3.4, 13.8 Hz, 1H), 3.28-3.21 (m, 1H), 3.17-3.09 (m, 2H), 3.01-2.83 (m, 2H), 1.64-1.46 (m, 3H), 1.38-1.25 (m, 3H).

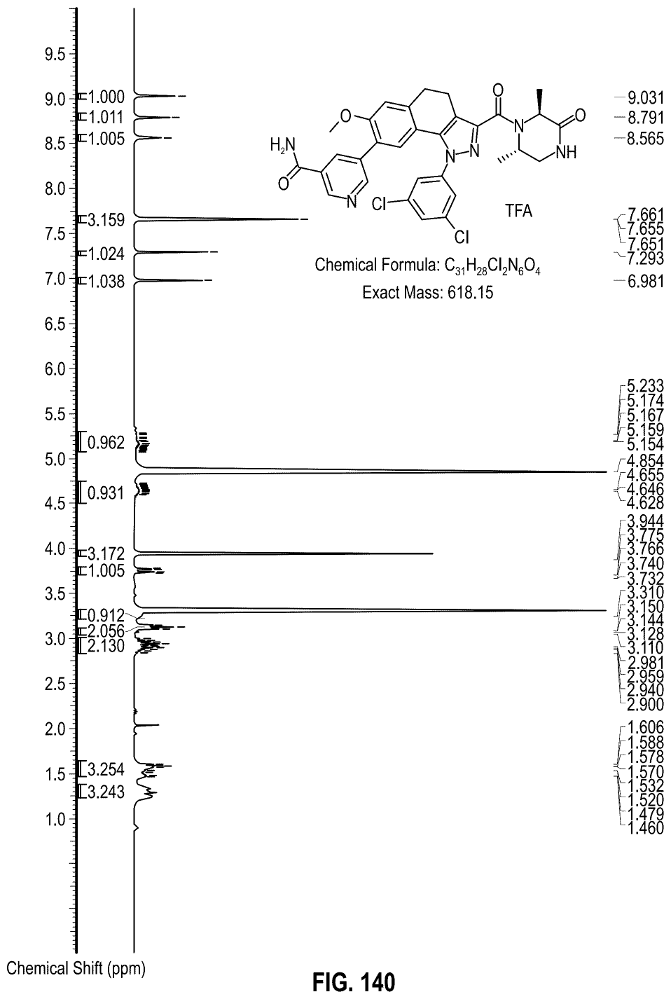

FIG. 140 shows the nuclear magnetic resonance of Compound 8-87.

### Synthesis of Compound 8-89

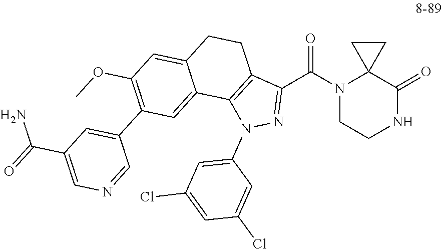 [SMILES: COc1cc2c(cc1-c1cncc(C(N)=O)c1)-c1c(c(C(=O)N3CCNC(=O)C34CC4)nn1-c1cc(Cl)cc(Cl)c1)CC2]

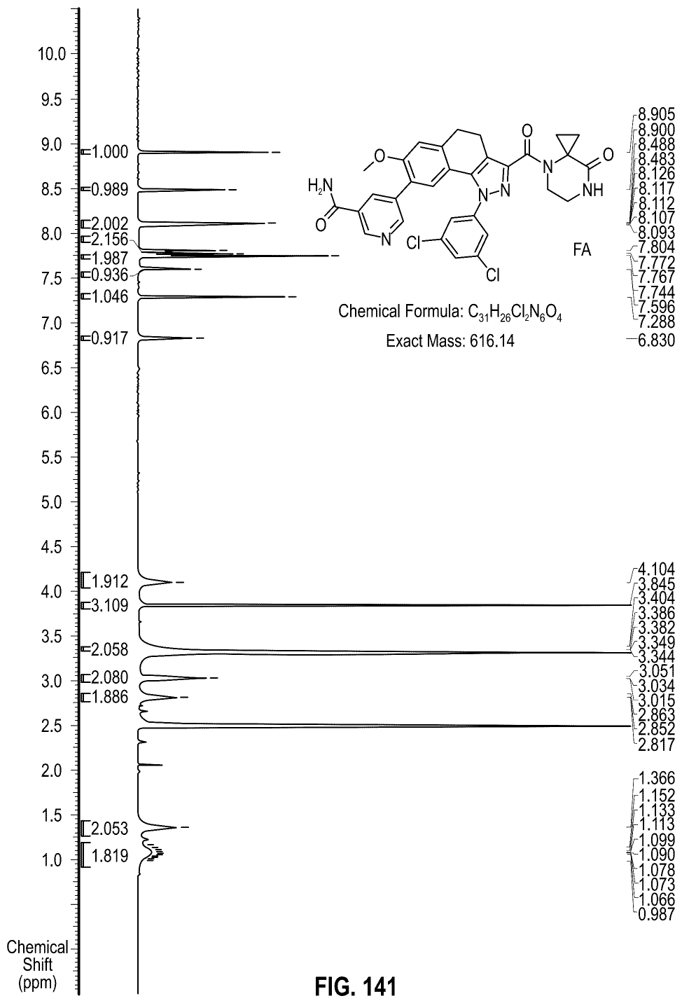

Compound 8-89 was synthesized according to a procedure similar to Reaction scheme 27. LCMS (ESI): m/z[M+H] calcd for C32H28Cl2N6O6: 617.14; found: 617.1. 1H NMR (400 MHz, DMSO-d6) δ=8.90 (d, J=2.0 Hz, 1H), 8.49 (d, J=2.1 Hz, 1H), 8.14-8.06 (m, 2H), 7.83-7.76 (m, 2H), 7.74 (d, J=1.6 Hz, 2H), 7.60 (s, 1H), 7.29 (s, 1H), 6.83 (s, 1H), 4.10 (br s, 2H), 3.84 (s, 3H), 3.39-3.34 (m, 2H), 3.03 (br t, J=7.1 Hz, 2H), 2.87-2.76 (m, 2H), 1.37 (br s, 2H), 1.20-0.92 (m, 2H). FIG. 141 shows the nuclear magnetic resonance of Compound 8-89.

### Example 9

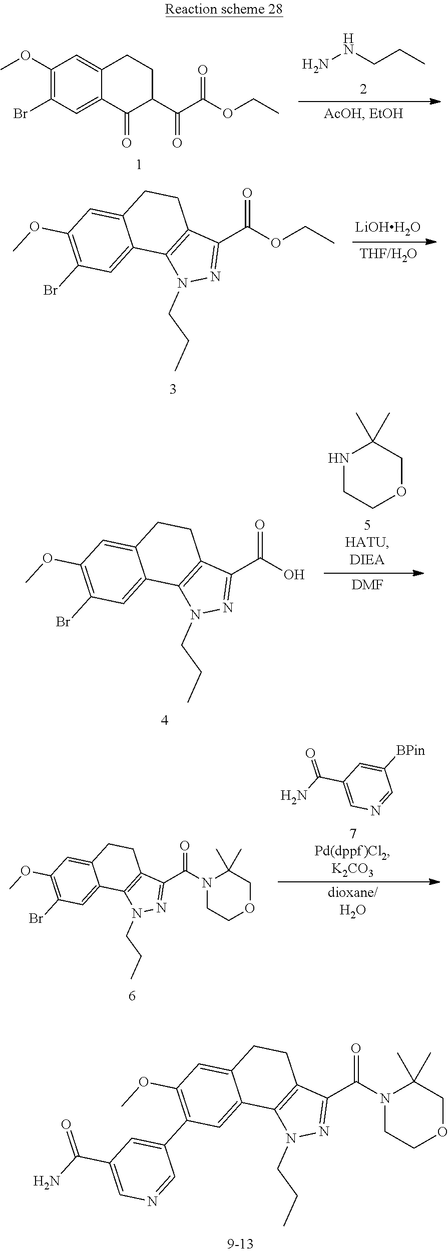

### Synthesis of ethyl 8-bromo-7-methoxy-1-propyl-4,5-dihydrobenzo[g]indazole-3-carboxylate

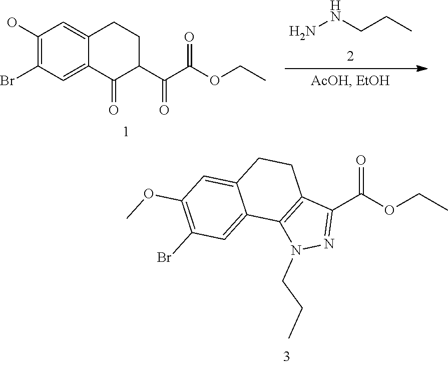

A mixture of ethyl 2-(7-bromo-6-methoxy-1-oxo-tetralin-2-yl)-2-oxo-acetate (0.2 g, 563.09 μmol, 1 eq), propylhydrazine;hydrochloride (68.50 mg, 619.40 μmol, 1.1 eq) and AcOH (338.15 mg, 5.63 mmol, 322.35 μL, 10 eq) in EtOH (3 mL) was stirred at 80° C. for 2 hr under N2 atmosphere. The residue was concentrated. The residue was purified by flash silica gel chromatography (ISCO®; 25 g SepaFlash® Silica Flash Column, Eluent of 85-75% Petroleum ether/Ethyl acetategradient @60 mL/min). Compound ethyl 8-bromo-7-methoxy-1-propyl-4,5-dihydrobenzo[g]indazole-3-carboxylate (168 mg, 418.64 μmol, 82.32% yield, 98% purity) was obtained as a light yellow solid. 1H NMR (400 MHz, DMSO-d6) δ=7.73 (s, 1H), 7.21 (s, 1H), 4.42 (t, J=7.2 Hz, 2H), 4.28 (m, 2H), 3.89 (s, 3H), 2.87 (s, 4H), 1.82 (m, 2H), 1.30 (t, J=7.2 Hz, 3H), 0.88 (t, J=7.2 Hz, 3H).

### Synthesis of 8-bromo-7-methoxy-1-propyl-4,5-dihydrobenzo[g]indazole-3-carboxylic acid

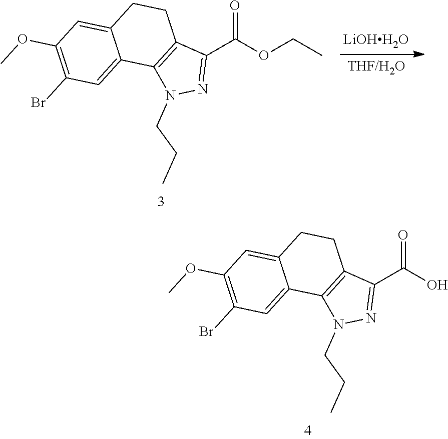

A mixture of ethyl 8-bromo-7-methoxy-1-propyl-4,5-dihydrobenzo[g]indazole-3-carboxylate (168 mg, 427.18 μmol, 1 eq), LiOH·H₂O (71.70 mg, 1.71 mmol, 1 mL, 4 eq), in THF (1 mL) and EtOH (1 mL), then the mixture was stirred at 25° C. for 16 hr under N2 atmosphere. The pH of the mixture was adjusted to 4-5 with hydrochloric acid (1 N) and filtered, the cake was washed with ethyl acetate. Compound 8-bromo-7-methoxy-1-propyl-4,5-dihydrobenzo[g]indazole-3-carboxylic acid (132 mg, 350.58 μmol, 82.07% yield, 97% purity) was obtained as a light yellow solid.

### Synthesis of (8-bromo-7-methoxy-1-propyl-4,5-dihydrobenzo[g]indazol-3-yl)-(3,3-dimethylmorpholin-4-yl)methanone

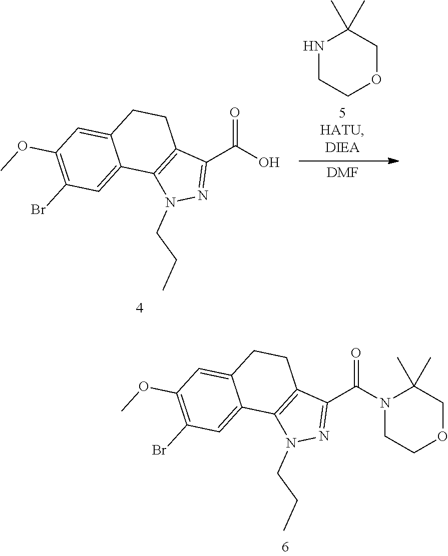

To a solution of 8-bromo-7-methoxy-1-propyl-4,5-dihydrobenzo[g]indazole-3-carboxylic acid (132 mg, 361.42 μmol, 1 eq) in DMF (3 mL) was added HATU (206.14 mg, 542.14 μmol, 1.5 eq)and DIEA (140.13 mg, 1.08 mmol, 188.86 μL, 3 eq)at 25° C. After addition, the mixture was stirred at this temperature for 0.5 hr, and then 3,3-dimethylmorpholine (54.11 mg, 469.85 μmol, 1.3 eq) was added at 25° C. The resulting mixture was stirred at 25° C. for 1.5 hr. The mixture was adjusted pH to 4-5 with hydrochloric acid (1 N) and extracted with ethyl acetate (3*20 mL). The organic phase was separated, washed with Saturated sodium chloride solution (10 mL), dried over Na₂SO₄, filtered and concentrated under reduced pressure to give a residue. The residue was purified by flash silica gel chromatography (ISCO®; 25 g SepaFlash® Silica Flash Column, Eluent of 70-80% Petroleum ether/Ethyl acetategradient @40 mL/min). Compound (8-bromo-7-methoxy-1-propyl-4,5-dihydrobenzo[g]indazol-3-yl)-(3,3-dimethylmorpholin-4-yl)methanone (120 mg, 241.36 μmol, 66.78% yield, 93% purity)was obtained as a light yellow solid.

### Synthesis of 5-[3-(3,3-dimethylmorpholine-4-carbonyl)-7-methoxy-1-propyl-4,5-dihydrobenzo[g]indazol-8-yl]pyridine-3-carboxamide

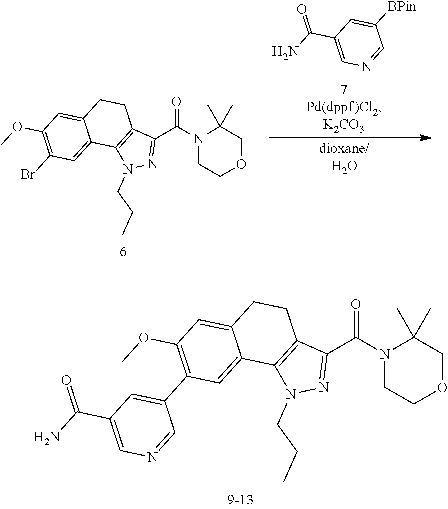

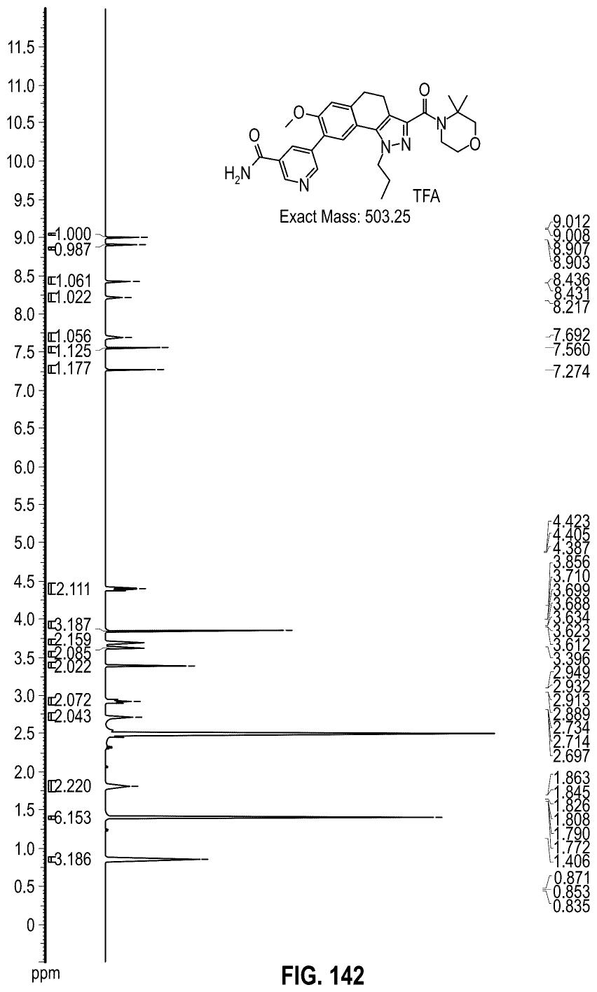

A mixture of (8-bromo-7-methoxy-1-propyl-4,5-dihydrobenzo[g]indazol-3-yl)-(3,3-dimethylmorpholin-4-yl)methanone (120 mg, 259.53 μmol, 1 eq), 5-(4,4,5,5-tetramethyl-1,3,2-dioxaborolan-2-yl)pyridine-3-carboxamide (70.82 mg, 285.48 mol, 1.1 eq), Pd(dppf)C₁₂ (37.98 mg, 51.91 g mol, 0.2 eq), K2CO3 (71.74 mg, 519.05 y mol, 2 eq)in dioxane (3 mL)and H2O (0.3 mL)was degassed and purged with N2 for 3 times, and then the mixture was stirred at 60° C. for 16 hr under N2 atmosphere. The mixture was poured into water (5 mL) and extracted with ethyl acetate (3*3 mL). The organic phase was separated, washed with Saturated sodium chloride solution (2 mL), dried over Na2SO4, filtered and concentrated under reduced pressure to give a residue. The residue was purified by prep-HPLC (column: Phenomenex Luna C18 150*25 mm*10 um;mobile phase: [water(TFA)-ACN];gradient:26%-56% B over 9 min). Compound 5-[3-(3,3-dimethylmorpholine-4-carbonyl)-7-methoxy-1-propyl-4,5-dihydrobenzo[g]indazol-8-yl]pyridine-3-carboxamide (82.67 mg, 131.18 μmol, 50.54% yield, 98% purity, TFA) was obtained as an off-white solid. LCMS (ESI): m/z [M+H] calcd for C28H33N5O4: 504.25; found: 504.2. ¹H NMR (400 MHz, DMSO-d6)δ=9.01 (d, J=1.6 Hz, 1H), 8.90 (d, J=1.6 Hz, 1H), 8.43 (d, J=2.0 Hz, 1H), 8.22 (s, 1H), 7.69 (s, 1H), 7.56 (s, 1H), 7.27 (s, 1H), 4.40 (t, J=7.2 Hz, 2H), 3.86 (s, 3H), 3.74-3.67 (m, 2H), 3.66-3.59 (m, 2H), 3.40 (s, 2H), 2.99-2.88 (m, 2H), 2.77-2.68 (m, 2H), 1.82 (m, 2H), 1.41 (s, 6H), 0.85 (t, J=7.2 Hz, 3H). FIG. 142 shows the nuclear magnetic resonance of Compound 9-13.

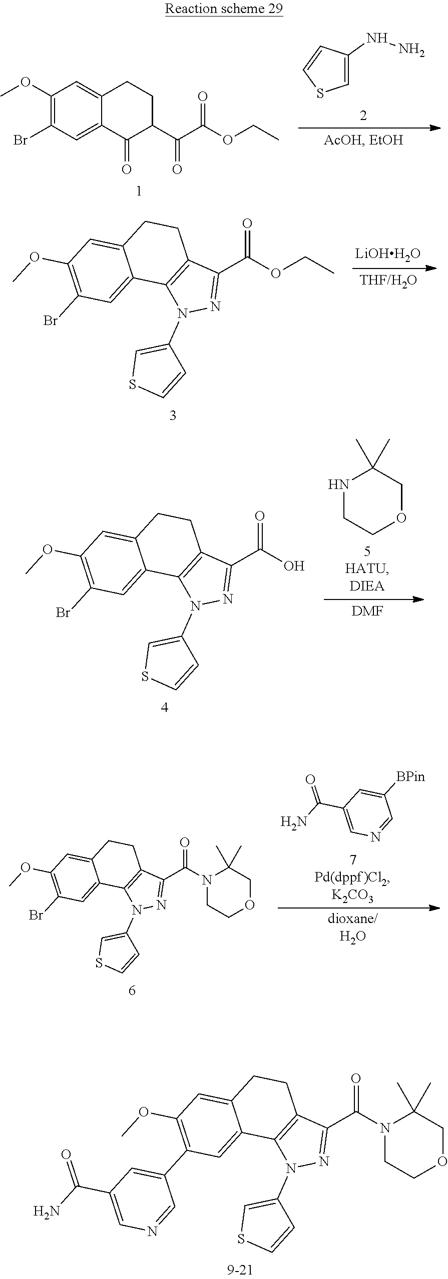

### Synthesis of ethyl 8-bromo-7-methoxy-1-(thiophen-3-yl)-4,5-dihydro-1H-benzo[g]indazole-3-carboxylate

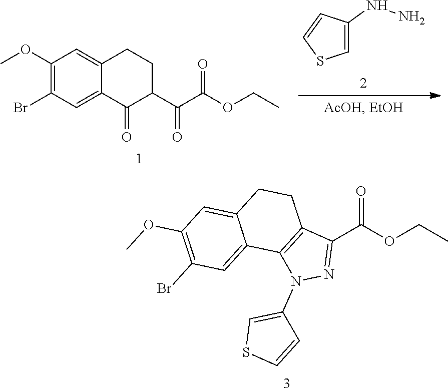

A mixture of ethyl 2-(7-bromo-6-methoxy-1-oxo-tetralin-2-yl)-2-oxo-acetate (0.2 g, 563.09 μmol, 1 eq), 3-thienylhydrazine (70.72 mg, 619.40 μmol, 1.1 eq) and AcOH (338.15 mg, 5.63 mmol, 322.35 μL, 10 eq) in EtOH (3 mL) was stirred at 80° C. for 2 hr under N2 atmosphere. The residue was concentrated. The residue was purified by flash silica gel chromatography (ISCO®; 25 g SepaFlash® Silica Flash Column, Eluent of 85-75% Petroleum ether/Ethyl acetategradient @60 mL/min). Compound ethyl 8-bromo-7-methoxy-1-(thiophen-3-yl)-4,5-dihydro-1H-benzo[g]indazole-3-carboxylate (180 mg, 398.78 μmol, 86.40% yield) was obtained as a gray solid. 1H NMR (400 MHz, DMSO-d6)δ=7.92 (m, 1H), 7.84 (m, 1H), 7.29 (m, 1H), 7.19 (s, 1H), 6.78 (s, 1H), 4.31 (m, 2H), 3.86 (s, 3H), 2.96 (s, 4H), 1.31 (t, J=7.2 Hz, 3H).

### Synthesis of 8-bromo-7-methoxy-1-(3-thienyl)-4,5-dihydrobenzo[g]indazole-3-carboxylic acid

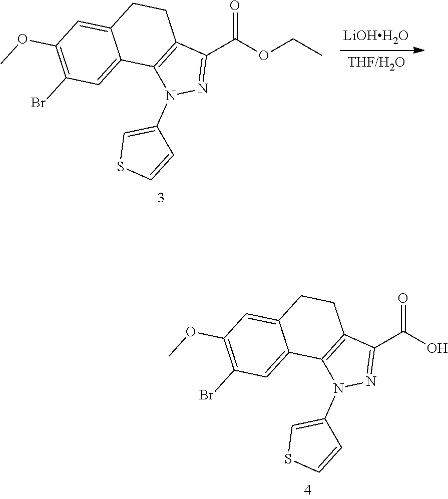

A mixture of ethyl 8-bromo-7-methoxy-1-(3-thienyl)-4,5-dihydrobenzo[g]indazole-3-carboxylate (180 mg, 415.40 μmol, 1 eq), LiOH·H2O (69.72 mg, 1.66 mmol, 1 mL, 4 eq), in THF (1 mL) and EtOH (1 mL) was stirred at 25° C. for 16 hr under N₂ atmosphere. The pH of the mixture was adjusted to 4-5 with hydrochloric acid (1 N) and the resulting mixture was extracted with ethyl acetate (3*20 mL), The organic phase was separated, washed with saturated sodium chloride solution (10 mL), dried over Na2SO4, filtered and concentrated under reduced pressure to give a residue. Compound 8-bromo-7-methoxy-1-(3-thienyl)-4,5-dihydrobenzo[g]indazole-3-carboxylic acid (133 mg, 328.18 μmol, 79.00% yield) was obtained as a light yellow solid.

### Synthesis of (8-bromo-7-methoxy-1-(thiophen-3-yl)-4,5-dihydro-1H-benzo[g]indazol-3-yl)(3,3-dimethylmorpholino)methanone

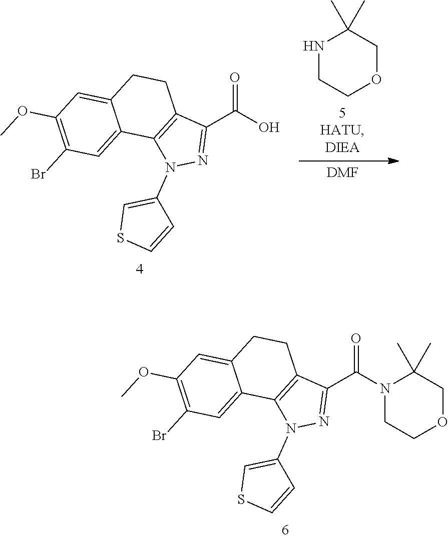

To a solution of 8-bromo-7-methoxy-1-(3-thienyl)-4,5-dihydrobenzo[g]indazole-3-carboxylic acid (133 mg, 328.18 μmol, 1 eq) in DMF (3 mL) was added HATU (187.18 mg, 492.27 μmol, 1.5 eq) and DIEA (127.24 mg, 984.54 μmol, 171.49 μL, 3 eq). After addition, the mixture was stirred at this temperature for 0.5 hr, and then 3,3-dimethylmorpholine (49.14 mg, 426.63 μmol, 1.3 eq) was added at 25° C. The resulting mixture was stirred at 25° C. for 1.5 hr. The pH of the mixture was adjusted to 4-5 with hydrochloric acid (1 N), the resulting mixture was extracted with ethyl acetate (3*20 mL). The organic phase was separated, washed with Saturated sodium chloride solution (10 mL), dried over Na2SO4, filtered and concentrated under reduced pressure to give a residue. The residue was purified by flash silica gel chromatography (ISCO®; 25 g SepaFlash® Silica Flash Column, Eluent of 70-80% Petroleum ether/Ethyl acetategradient @40 mL/min). Compound [8-bromo-7-methoxy-1-(3-thienyl)-4,5-dihydrobenzo[g]indazol-3-yl]-(3,3-dimethylmorpholin-4-yl)methanone (132 mg, 223.32 μmol, 68.05% yield, 85% purity) was obtained as a light yellow solid. LCMS (ESI): m/z [M+H] calcd for C23H24N3O3BrS: 504.07; found: 504.1.

### Synthesis of Compound 9-21

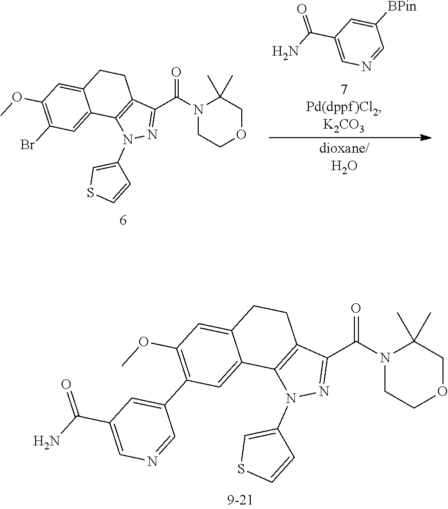

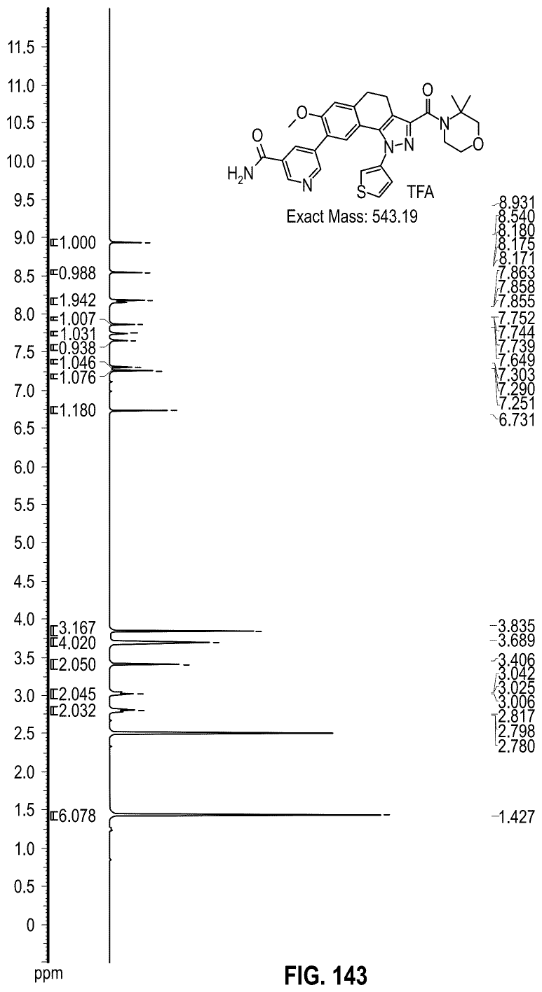

A mixture of [8-bromo-7-methoxy-1-(3-thienyl)-4,5-dihydrobenzo[g]indazol-3-yl]-(3,3-dimethylmorpholin-4-yl)methanone (132 mg, 262.73 μmol, 1 eq), 5-(4,4,5,5-tetramethyl-1,3,2-dioxaborolan-2-yl)pyridine-3-carboxamide (84.73 mg, 341.54 μmol, 1.3 eq), Pd(dppf)Cl2 (38.45 mg, 52.55 μmol, 0.2 eq), K₂CO₃ (72.62 mg, 525.45 μmol, 2 eq)in dioxane (3 mL) and H2O (0.3 mL) was degassed and purged with N2 for 3 times, and then the mixture was stirred at 60° C. for 16 hr under N2 atmosphere. The mixture was poured into water (5 mL) and extracted with ethyl acetate (3*3 mL ). The organic phase was separated, washed with saturated sodium chloride solution (2 mL), dried over Na2SO4, filtered and concentrated under reduced pressure to give a residue. The residue was purified by prep-HPLC (column: Phenomenex Luna C18 150*25 mm*10 um;mobile phase: [water(TFA)-ACN];gradient:30%-60% B over 9 min). Compound 5-[3-(3,3-dimethylmorpholine-4-carbonyl)-7-methoxy-1-(3-thienyl)-4,5-dihydrobenzo[g]indazol-8-yl]pyridine-3-carboxamide (65.91 mg, 118.81 μmol, 45.22% yield, 98% purity) was obtained as an off-white solid. LCMS (ESI): m/z [M+H] calcd for C29H29N5O4S: 544.19; found: 544.2. ¹H NMR (400 MHz, DMSO-d6)δ=8.93 (d, J=2.0 Hz, 1H), 8.54 (d, J=1.6 Hz, 1H), 8.21-8.12 (m, 2H), 7.86 (dd, J=1.2, 3.2 Hz, 1H), 7.74 (m, 1H), 7.65 (s, 1H), 7.30 (dd, J=1.2, 5.2 Hz, 1H), 7.25 (s, 1H), 6.73 (s, 1H), 3.83 (s, 3H), 3.69 (s, 4H), 3.41 (s, 2H), 3.02 (m, 2H), 2.86-2.75 (m, 2H), 1.43 (s, 6H). FIG. 143 shows the nuclear magnetic resonance of Compound 9-21.

### Synthesis of Compound 9-04

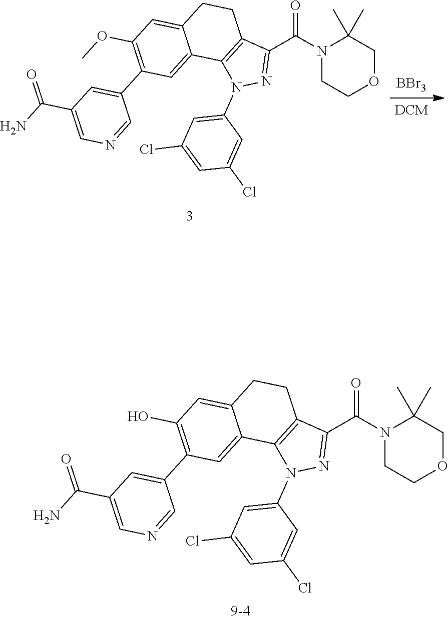

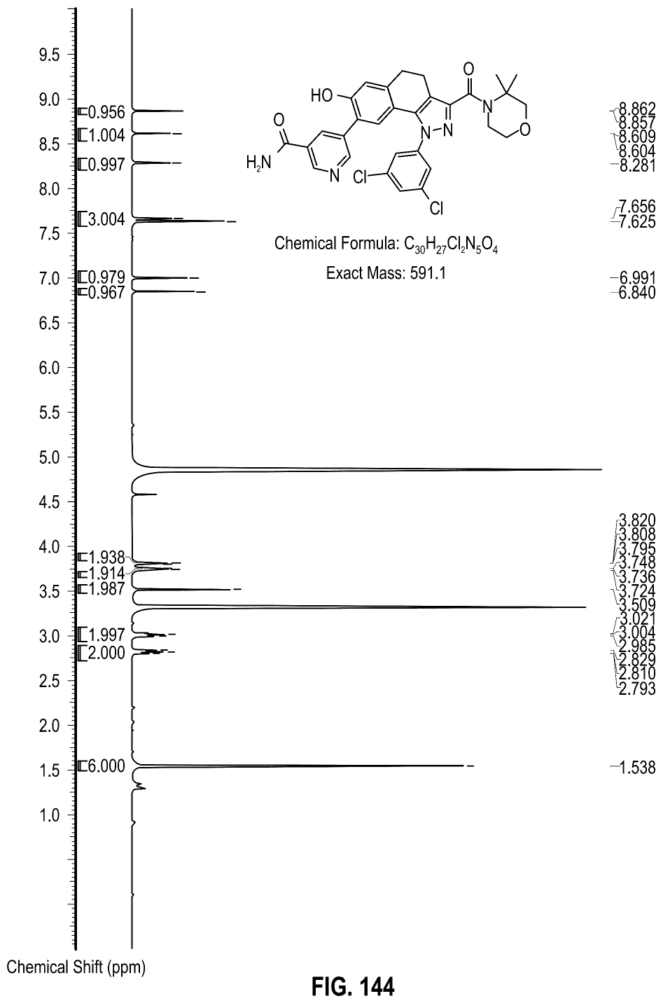

To a mixture of 5-[1-(3,5-dichlorophenyl)-3-(3,3-dimethylmorpholine-4-carbonyl)-7-methoxy-4, 5-dihydrobenzo[g]indazol-8-yl]pyridine-3-carboxamide (20 mg, 32.98 μmol, 1 eq) in DCM (2 mL) was added BBr3 (24.78 mg, 98.93 μmol, 9.53 μL, 3 eq)at 0° C. The mixture was stirred at 0° C. for 1 h. The mixture was poured into sat. NaHCO3 (4 mL), the resulting mixture was extracted with DCM (3 mL*3). The combined organic phase was dried over anhydrous Na2SO4 and filtered. The filtrate was concentrated in vacuum to give a residue. The residue was purified by prep-HPLC (column: Waters Xbridge 150*25 mm*5 um;mobile phase:[water (NH4HCO3)-ACN];gradient: 30%-60% B over 9 min), followed by lyophilization to give 5-[1-(3,5-dichlorophenyl)-3-(3,3-dimethylmorpholine-4-carbonyl)-7-hydroxy-4,5-dihydrobenzo[g]indazol-8-yl]pyridine-3-carboxamide (5.04 mg, 8.51 μmol, 25.80% yield, 100% purity) as a white solid. LCMS (ESI): m/z [M+H] calcd for C18H20O3N3ClF: 592.14; found: 592.1. 1H NMR (400 MHz, METHANOL-d4)δ=8.86 (d, J=2.00 Hz, 1H), 8.61 (d, J=2.00 Hz, 1H), 8.28 (s, 1H), 7.74-7.57 (m, 3H), 6.99 (s, 1H), 6.84 (s, 1H), 3.85-3.78 (m, 2H), 3.76-3.70 (m, 2H), 3.51 (s, 2H), 3.08-2.93 (m, 2H), 2.88-2.72 (m, 2H), 1.54 (s, 6H). FIG. 144 shows the nuclear magnetic resonance of Compound 9-4.

### Synthesis of Compound 9-5

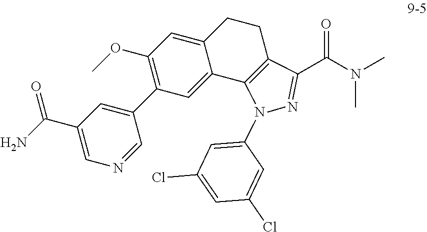 [SMILES: COc1cc2c(cc1-c1cncc(C(N)=O)c1)-c1c(c(C(=O)N(C)C)nn1-c1cc(Cl)cc(Cl)c1)CC2]

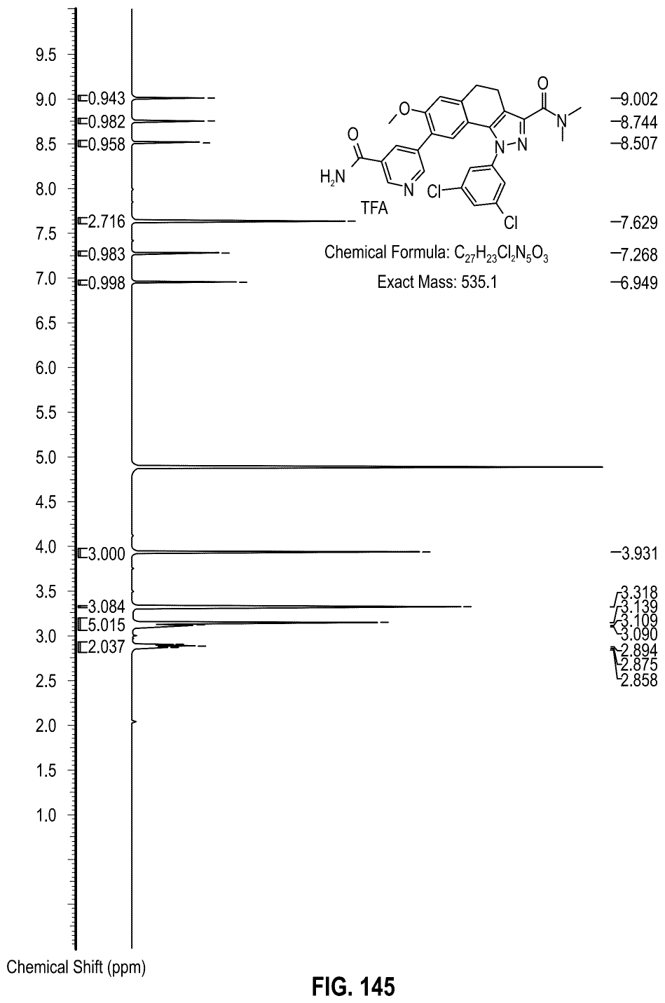

Compound 9-05 was synthesized according to a procedure similar to Example 9. LCMS (ESI): m/z [M+H] calcd for C₂₉H₂₄N₅O₅Cl₂F₃: 536.12; found: 536.1. ¹H NMR (400 MHz, METHANOL-d₄)δ=9.00 (s, 1H), 8.74 (s, 1H), 8.51 (s, 1H), 7.63 (s, 3H), 7.27 (s, 1H), 6.95 (s, 1H), 3.93 (s, 3H), 3.32 (s, 3H), 3.05-3.19 (m, 5H), 2.88 (br t, J=7.15 Hz, 2H). FIG. 145 shows the nuclear magnetic resonance of Compound 9-5.

### Synthesis of Compound 9-11

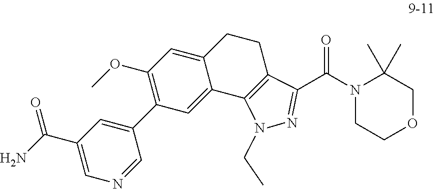 [SMILES: CCn1nc(C(=O)N2CCOCC2(C)C)c2c1-c1cc(-c3cncc(C(N)=O)c3)c(OC)cc1CC2]

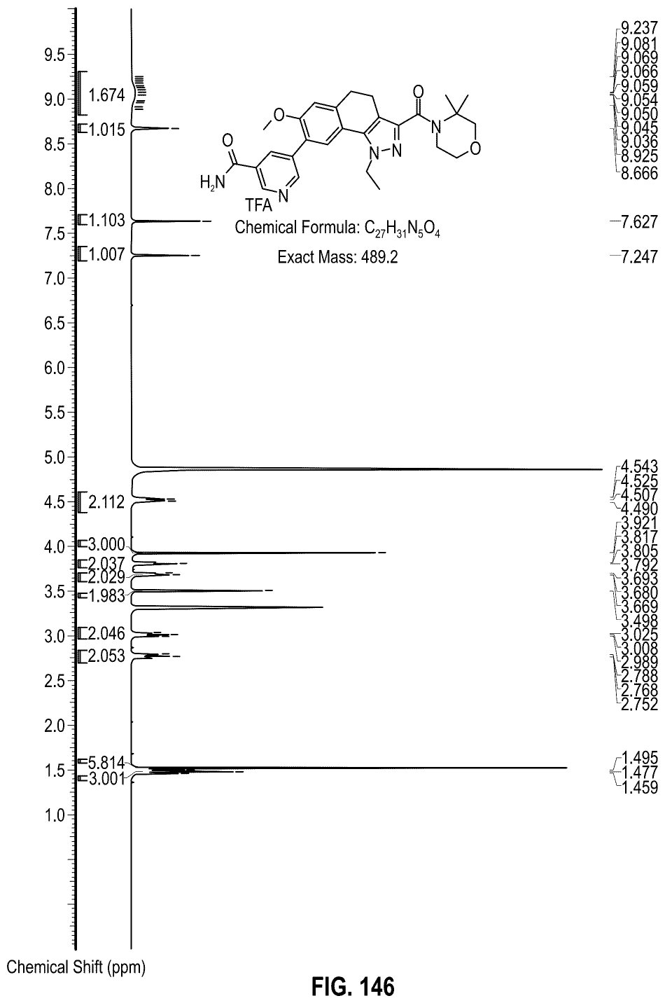

Compound 9-05 was synthesized according to a procedure similar to Example 9. LCMS (ESI): m/z [M+H] calcd for C₂₇H₃₁N₅O₄: 490.24; found: 490.2. ¹H NMR (400 MHz, METHANOL-d₄)δ ppm 1.48 (t, J=7.19 Hz, 3H)1.50-1.55 (m, 6H)2.69-2.83 (m, 2H)2.96-3.08 (m, 2H)3.50 (s, 2H)3.62-3.72 (m, 2H)3.76-3.85 (m, 2H)3.92 (s, 3H)4.52 (q, J=7.17 Hz, 2H)7.25 (s, 1H)7.63 (s, 1H)8.67 (s, 1H)8.82-9.30 (m, 2H). FIG. 146 shows the nuclear magnetic resonance of Compound 9-11.

### Synthesis of Compound 9-14

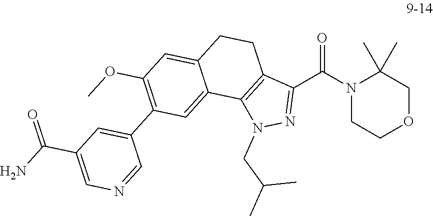 [SMILES: COc1cc2c(cc1-c1cncc(C(N)=O)c1)-c1c(c(C(=O)N3CCOCC3(C)C)nn1CC(C)C)CC2]

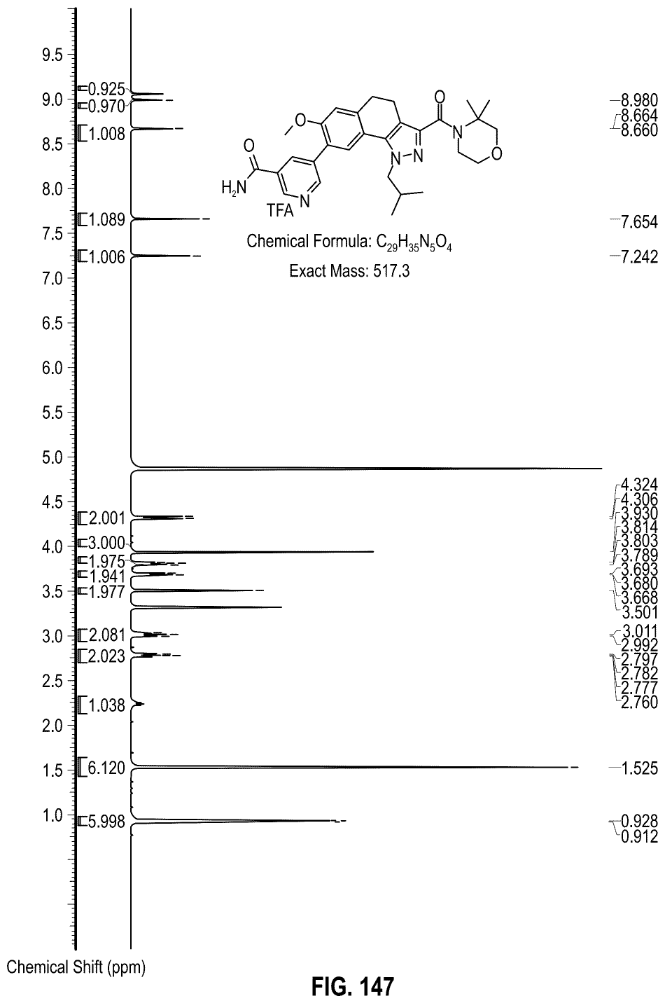

Compound 9-14 was synthesized according to a procedure similar to Example 9. LCMS (ESI): m/z [M+H] calcd for C29H35N5O4: 518.27; found:518.3. 1H NMR (400 MHz, METHANOL-d4)δ ppm 0.92 (d, J=6.63 Hz, 6H)1.53 (s, 6H)2.12-2.32 (m, 1H)2.70-2.84 (m, 2H)2.93-3.07 (m, 2H)3.50 (s, 2H)3.64-3.72 (m, 2H)3.76-3.84 (m, 2H)3.93 (s, 3H)4.31 (d, J=7.38 Hz, 2H)7.24 (s, 1H)7.65 (s, 1H)8.52-8.70 (m, 1H)8.98 (d, J=1.50 Hz, 1H)9.03-9.07 (m, 1H). FIG. 147 shows the nuclear magnetic resonance of Compound 9-14.

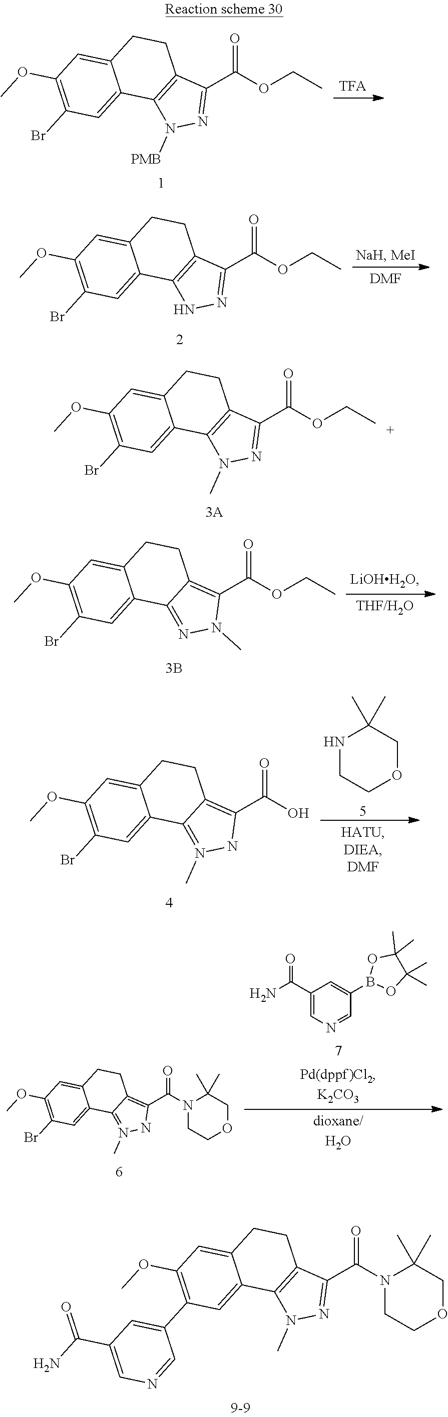

### Synthesis of ethyl 8-bromo-7-methoxy-4,5-dihydro-1H-benzo[g]indazole-3-carboxylate

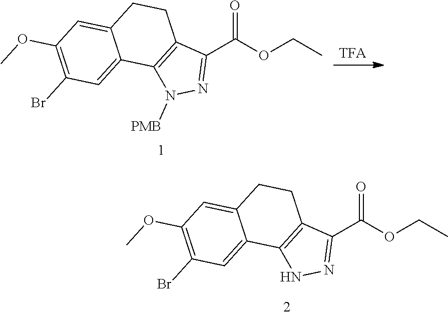

A mixture of ethyl 8-bromo-7-methoxy-1-[(4-methoxyphenyl)methyl]-4,5-dihydrobenzo[g]indazole-3-carboxylate (3 g, 6.36 mmol, 1 eq) in TFA (30 mL) was stirred at 70° C. for 16 h. The mixture was concentrated under vacuum to give a residue. The residue was triturated with (Petroleum ether:ethyl acetate=10:1) (50 mL), the resulting mixture was filtered and filtered cake was concentrated under vacuum to give ethyl 8-bromo-7-methoxy-4,5-dihydro-1H-benzo[g]indazole-3-carboxylate (2.2 g, 6.26 mmol, 98.42% yield) as a yellow solid.

### Synthesis of ethyl 8-bromo-7-methoxy-1-methyl-4,5-dihydrobenzo[g]indazole-3-carboxylate (3A); ethyl 8-bromo-7-methoxy-2-methyl-4,5-dihydrobenzo[g]indazole-3-carboxylate(3B)

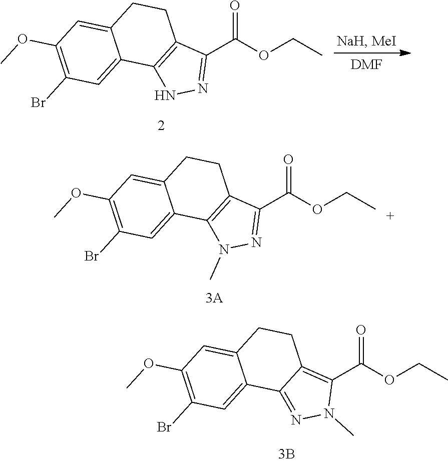

To a mixture of ethyl 8-bromo-7-methoxy-4,5-dihydro-1H-benzo[g]indazole-3-carboxylate (300 mg, 854.23 μmol, 1 eq) in THF (3 mL) was added NaH (44.42 mg, 1.11 mmol, 60% purity, 1.3 eq), the mixture was stirred at 25° C. for 15 min, Mel (181.87 mg, 1.28 mmol, 79.77 μL, 1.5 eq), the mixture was stirred at 25° C. for 16 h. The mixture was quenched by H2O (5 mL), the resulting mixture was extracted with ethyl acetate(5 mL*3), the combined organic phase was concentrated under vacuum to give a residue. The residue was purified by prep-HPLC (column: Phenomenex Luna C18 150*25 mm*10 um;mobile phase:[water(TFA)-ACN];gradient:58%-88% B over 9 min) followed by lyophilization to give ethyl 8-bromo-7-methoxy-1-methyl-4,5-dihydrobenzo[g]indazole-3-carboxylate (3A) (50 mg, 136.90 μmol, 16.03% yield) as a white solid and ethyl 8-bromo-7-methoxy-2-methyl-4,5-dihydrobenzo[g]indazole-3-carboxylate (70 mg, 191.66 μmol, 22.44% yield) as a white solid.

Compound 3A: 1H NMR (400 MHz, CHLOROFORM-d) δ=7.73 (s, 1H), 6.91 (s, 1H), 4.43 (q, J=7.1 Hz, 2H), 4.21 (s, 3H), 3.95 (s, 3H), 3.06-2.99 (m, 2H), 2.96-2.85 (m, 2H), 1.43 (t, J=7.1 Hz, 3H).

Compound 3B: ¹H NMR (400 MHz, CHLOROFORM-d) δ=8.02 (s, 1H), 6.78 (s, 1H), 4.39 (q, J=7.1 Hz, 2H), 4.19 (s, 3H), 3.92 (s, 3H), 3.07-2.97 (m, 2H), 2.94-2.86 (m, 2H), 1.42 (t, J=7.1 Hz, 3H).

### Synthesis of 8-bromo-7-methoxy-1-methyl-4,5-dihydrobenzo[g]indazole-3-carboxylic acid

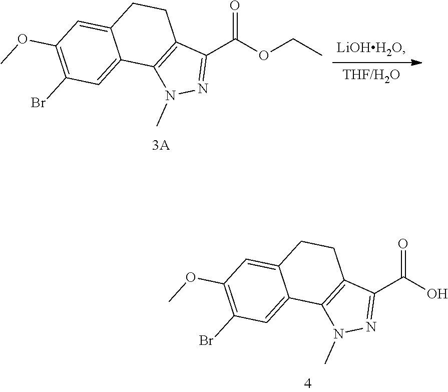

To a mixture of ethyl 8-bromo-7-methoxy-1-methyl-4,5-dihydrobenzo[g]indazole-3-carboxylate (50 mg, 136.90 μmol, 1 eq) in THF (2 mL) and MeOH (0.5 mL), H2O (1 mL) was added LiOH·H2O (17.23 mg, 410.71 μmol, 3 eq), the mixture was stirred at 25° C. for 16 h. The mixture was concentrated under vacuum to remove organic phase, the pH of the resulting mixture was adjusted to 5 by 1N HCl, a lot of solid was generated, the resulting mixture was filtered, the filtered cake was dried under vacuum to give 8-bromo-7-methoxy-1-methyl-4,5-dihydrobenzo[g]indazole-3-carboxylic acid (45 mg, 133.46 μmol, 97.49% yield) as a white solid.

### Synthesis of (8-bromo-7-methoxy-1-methyl-4,5-dihydrobenzo[g]indazol-3-yl)-(3,3-dimethylmorpholin-4-yl)methanone

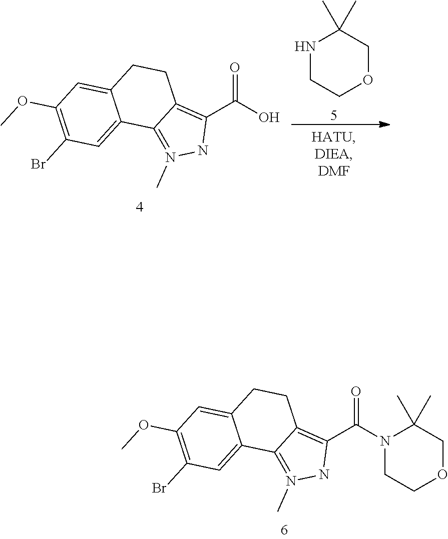

To a mixture of 8-bromo-7-methoxy-1-methyl-4,5-dihydrobenzo[g]indazole-3-carboxylic acid (45 mg, 133.46 μmol, 1 eq) in DMF (1 mL) was added HATU (60.90 mg, 160.16 μmol, 1.2 eq), DIEA (51.75 mg, 400.39 μmol, 69.74 μL, 3 eq), then 3,3-dimethylmorpholine (15.37 mg, 133.46 μmol, 1 eq) was added, the mixture was stirred at 25° C. for 1 h. The mixture was diluted with H2O (5 mL), a lot of solid was generated, the resulting mixture was filtered and filtered cake was dried under vacuum to give (8-bromo-7-methoxy-1-methyl-4,5-dihydrobenzo[g]indazol-3-yl)-(3,3-dimethylmorpholin-4-yl)methanone (55 mg, 126.63 μmol, 94.88% yield) as a brown solid.

### Synthesis of Compound 9-9

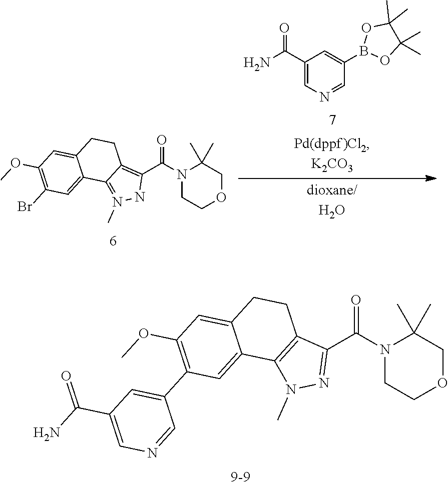

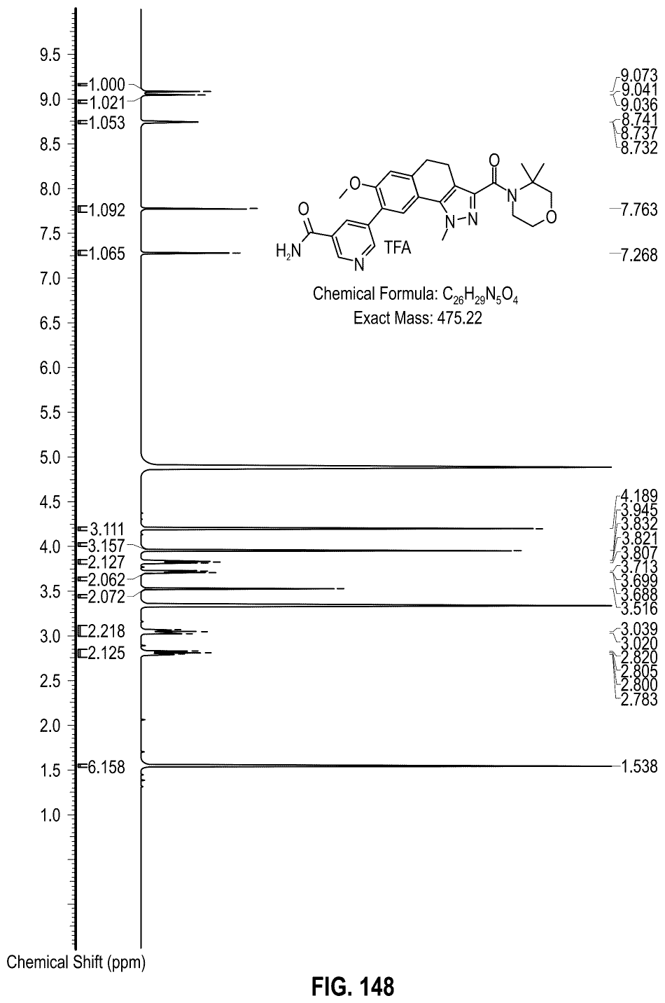

To a mixture of (8-bromo-7-methoxy-1-methyl-4,5-dihydrobenzo[g]indazol-3-yl)-(3,3-dimethylmorpholin-4-yl)methanone (40.00 mg, 92.10 μmol, 1 eq) in dioxane (2 mL) and H2O (0.5 mL) was added K2CO3 (12.73 mg, 92.10 μmol, 1 eq), Pd(dppf)Cl2 (6.74 mg, 9.21 μmol, 0.1 eq). The n 5-(4,4,5,5-tetramethyl-1,3,2-dioxaborolan-2-yl)pyridine-3-carboxamide (29.70 mg, 119.73 μmol, 1.3 eq) was added, the mixture was stirred at 60° C. for 16 h under N2. The mixture was concentrated under vacuum to give a residue. The residue was purified by prep-HPLC (column: Phenomenex Luna C18 150*25 mm*10 um;mobile phase:[water(TFA)-ACN];gradient:22%-52% B over 9 min) followed by lyophilization to give 5-[3-(3,3-dimethylmorpholine-4-carbonyl)-7-methoxy-1-methyl-4,5-dihydrobenzo[g]indazol-8-yl]pyridine-3-carboxamide (11.89 mg, 20.17 μmol, 21.90% yield, 100% purity, TFA) as a yellow solid. LCMS (ESI): m/z [M+H] calcd for C26H30N5O4:476.22; found: 476.2. 1H NMR (400 MHz, METHANOL-d4)δ=9.07 (d, J=1.8 Hz, 1H), 9.04 (d, J=1.9 Hz, 1H), 8.75-8.71 (m, 1H), 7.76 (s, 1H), 7.27 (s, 1H), 4.19 (s, 3H), 3.94 (s, 3H), 3.85-3.79 (m, 2H), 3.72-3.68 (m, 2H), 3.52 (s, 2H), 3.11-2.99 (m, 2H), 2.84-2.76 (m, 2H), 1.54 (s, 6H). FIG. 148 shows the nuclear magnetic resonance of Compound 9-9.

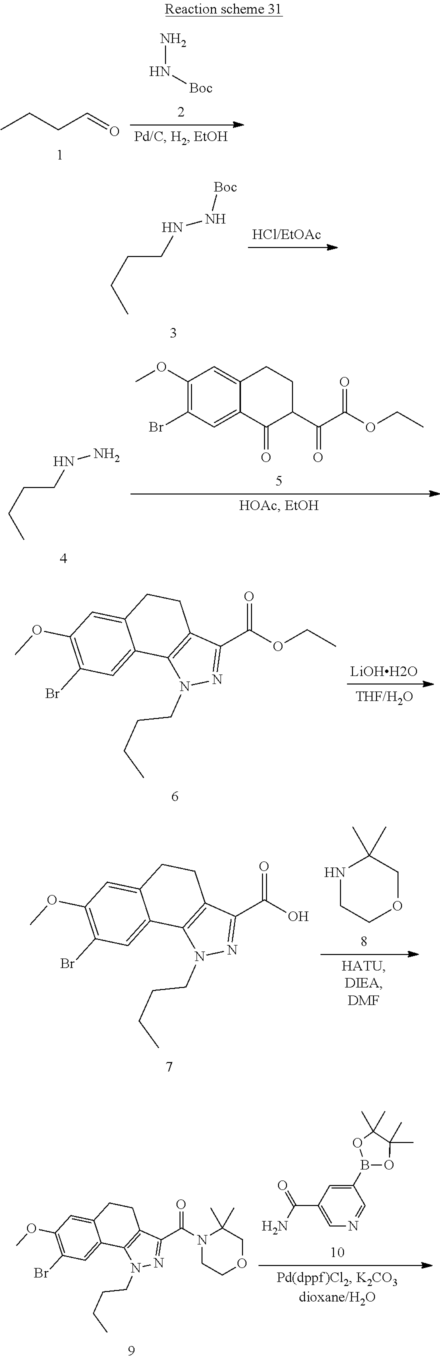

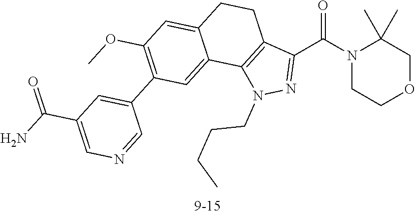 [SMILES: CCCCn1nc(C(=O)N2CCOCC2(C)C)c2c1-c1cc(-c3cncc(C(N)=O)c3)c(OC)cc1CC2]

### Synthesis of tert-butyl N-(butylamino)carbamate

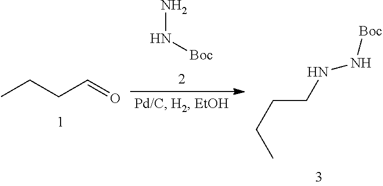

To a mixture of butanal (1 g, 13.87 mmol, 1.22 mL, 1 eq), tert-butyl N-aminocarbamate (3.67 g, 27.74 mmol, 2 eq) and tert-butyl N-aminocarbamate (3.67 g, 27.74 mmol, 2 eq) in EtOH (20 mL) was added Pd/C (1.48 g, 1.39 mmol, 10% purity, 0.1 eq) under N2 atmosphere. The mixture was degassed and purged with H2 for 3 times, and then the mixture was stirred at 25° C. for 16 h under H2 (40 Psi) atmosphere. The reaction mixture was filtered and concentrated under reduced pressure to give a residue. ¼ of the residue was purified by prep-HPLC (column: Phenomenex Luna C18 150*25 mm*10 um; mobile phase:[water (TFA)-ACN]; gradient: 28%-58% B over 9 min). Compound tert-butyl N-(butylamino)carbamate (186 mg, 987.96 μmol, 7.12% yield) was obtained as a colorless oil. Another ¾ of the residue as crude tert-butyl N-(butylamino)carbamate (2.1 g, crude)was obtained as a colorless oil. 1H NMR: (400 MHz, DMSO-d6)δ=8.80-8.43 (m, 1H), 2.72 (t, J=6.9 Hz, 2H), 1.43-1.23 (m, 13H), 0.86 (t, J=7.1 Hz, 3H).

### Synthesis of butylhydrazine

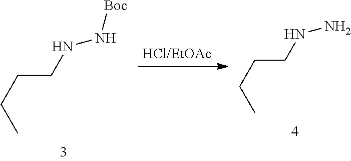

To a mixture of tert-butyl N-(butylamino)carbamate (180 mg, 956.09 μmol, 1 eq) in EtOAc (1 mL) was added HCl/EtOAc (2 M, 4.78 mL, 10 eq). The mixture was stirred at 25° C. for 2 h. The reaction mixture was and concentrated under reduced pressure to afford compound butylhydrazine (123 mg, crude, HCl) as a colorless oil. 1H NMR: (400 MHz, DMSO-d6)δ=2.92-2.84 (m, 2H), 1.57-1.48 (m, 2H), 1.31 (qd, J=7.4, 15.0 Hz, 2H), 0.87 (t, J=7.3 Hz, 3H).

### Synthesis of ethyl 8-bromo-1-butyl-7-methoxy-4, 5-dihydrobenzo[g]indazole-3-carboxylate

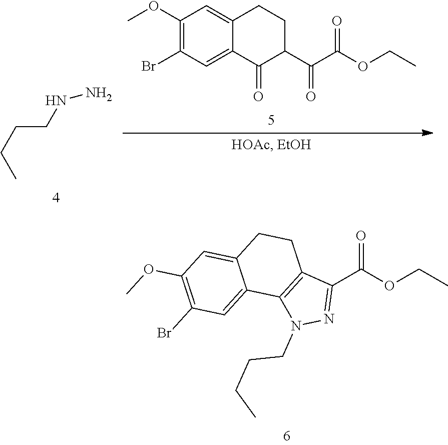

A mixture of butylhydrazine (70.17 mg, 563.09 μmol, 1 eq, HCl), ethyl 2-(7-bromo-6-methoxy-1-oxo-tetralin-2-yl)-2-oxo-acetate (200 mg, 563.09 μmol, 1 eq) and AcOH (1.35 g, 22.52 mmol, 1.29 mL, 40 eq) in t-BuOH (0.8 mL) was stirred at 80° C. for 16 h. The reaction mixture was concentrated under reduced pressure to give a residue. The residue was purified by column chromatography (SiO2, Petroleum ether/Ethyl acetate=1/0 to 3/1) to afford compound ethyl 8-bromo-1-butyl-7-methoxy-4,5-dihydrobenzo[g]indazole-3-carboxylate (190 mg, 466.49 μmol, 82.84% yield) as a yellow solid. 1H NMR: (400 MHz, METHANOL-d4) δ=7.73 (s, 1H), 7.10 (s, 1H), 4.49-4.42 (m, 2H), 4.37 (q, J=7.1 Hz, 2H), 3.92 (s, 3H), 2.97-2.93 (m, 2H), 2.92-2.86 (m, 2H), 1.87 (quin, J=7.5 Hz, 2H), 1.43-1.36 (m, 5H), 0.99 (t, J=7.4 Hz, 3H).

### Synthesis of 8-bromo-1-butyl-7-methoxy-4,5-dihydrobenzo[g]indazole-3-carboxylic acid

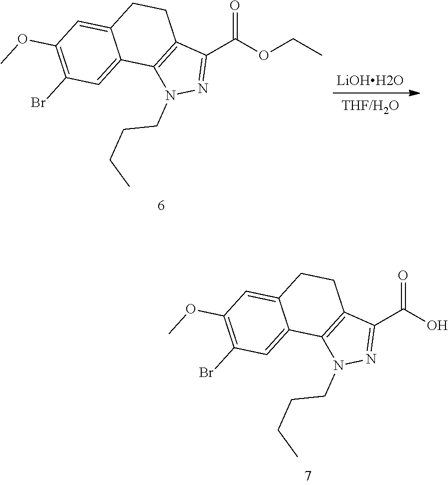

A mixture of ethyl 8-bromo-1-butyl-7-methoxy-4,5-dihydrobenzo[g]indazole-3-carboxylate (100 mg, 245.52 μmol, 1 eq) and LiOH. H2O (30.91 mg, 736.56 μmol, 3 eq) in EtOH (1 mL)and H2O (0.3 mL)was stirred at 25° C. for 18 h. The reaction mixture was concentrated under reduced pressure to remove EtOH, diluted with H2O (15 mL) and extracted with EtOAc (10 mL*2). The aqueous phase was adjusted pH=4 with 1 M HCl, a lot of solid precipitated out. The resulting mixture was filtered, the filter cake was concentrated under reduced pressure to afford compound 8-bromo-1-butyl-7-methoxy-4,5-dihydrobenzo[g]indazole-3-carboxylic acid (63 mg, crude) as a yellow solid.

### Synthesis of(8-bromo-1-butyl-7-methoxy-4,5-dihydrobenzo[g]indazol-3-yl)-(3, 3-dimethylmorpholin-4-yl)methanone

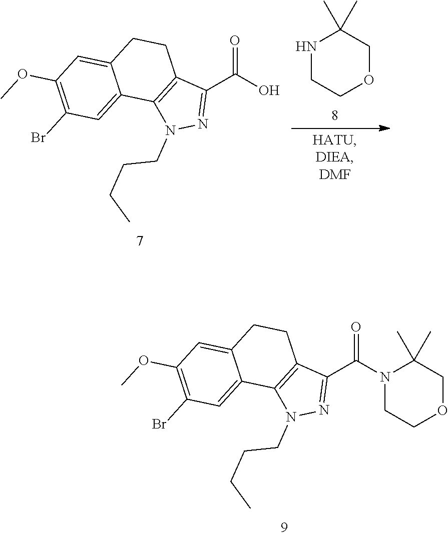

A mixture of 8-bromo-1-butyl-7-methoxy-4,5-dihydrobenzo[g]indazole-3-carboxylic acid (60 mg, 158.21 μmol, 1 eq), HATU (120.31 mg, 316.42 μmol, 2 eq) and DIEA (61.34 mg, 474.62 μmol, 82.67 μL, 3 eq) in DMF (1 mL) was stirred at 25° C. for 15 min. To the mixture was added 3, 3-dimethylmorpholine (21.87 mg, 189.85 μmol, 1.2 eq). The mixture was stirred at 25° C. for 16 h. The reaction mixture was diluted with H₂O (10 mL) and extracted with EtOAc (10 mL*3). The combined organic layers were dried over anhydrous Na₂SO₄, filtered and concentrated under reduced pressure to give a residue. The residue was purified by column chromatography (SiO₂, Petroleum ether/Ethyl acetate=1/0 to 3/1) and concentrated under reduced pressure to afford compound (8-bromo-1-butyl-7-methoxy-4,5-dihydrobenzo[g]indazol-3-yl)-(3,3-dimethylmorpholin-4-yl)methanone (43 mg, 90.26 μmol, 57.05% yield) as a brown solid.

### Synthesis of Compound 9-15

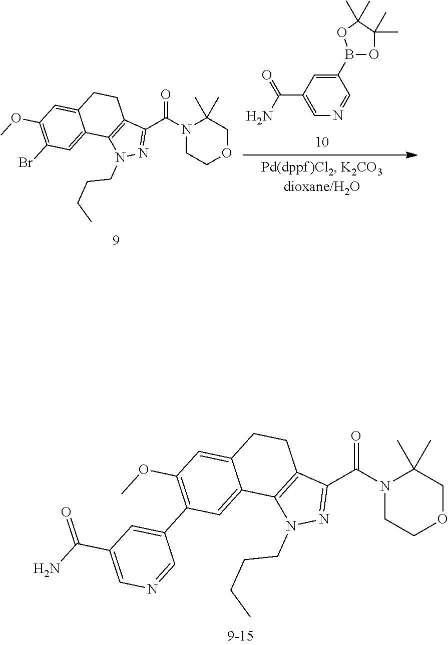 [SMILES: CCCCn1nc(C(=O)N2CCOCC2(C)C)c2c1-c1cc(Br)c(OC)cc1CC2 ; CC1(C)OB(c2cncc(C(N)=O)c2)OC1(C)C ; CCCCn1nc(C(=O)N2CCOCC2(C)C)c2c1-c1cc(-c3cncc(C(N)=O)c3)c(OC)cc1CC2]

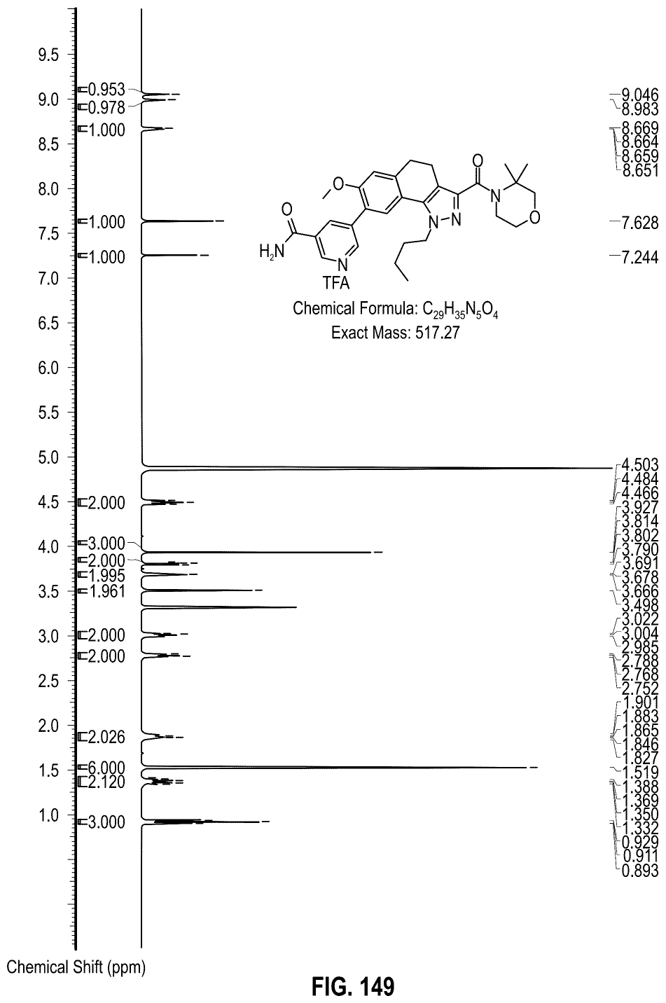

To a mixture of (8-bromo-1-butyl-7-methoxy-4,5-dihydrobenzo[g]indazol-3-yl)-(3,3-dimethylmorpholin-4-yl)methanone (40 mg, 83.96 μmol, 1 eq), 5-(4,4,5,5-tetramethyl-1,3,2-dioxaborolan-2-yl)pyridine-3-carboxamide (31.24 mg, 125.94 μmol, 1.5 eq)and K2CO3 (34.81 mg, 251.89 μmol, 3 eq)in dioxane (1 mL)and H2O (0.3 mL)was added Pd(dppf)Cl2 (6.14 mg, 8.40 μmol, 0.1 eq)under N2 atmosphere. The mixture was degassed and purged with N2 for 3 times, and then the mixture was stirred at 80° C. for 16 h under N2 atmosphere. The reaction mixture was diluted with H2O (10 mL) and extracted with EtOAc (10 mL*3). The combined organic layers were dried over anhydrous Na2SO4, filtered and concentrated under reduced pressure to give a residue. The residue was purified by prep-HPLC (column: Phenomenex Luna C18 150*25 mm*10Ium; mobile phase: [water (TFA)-ACN]; gradient: 30%-60% B over 9 min). Compound 5-[1-butyl-3-(3,3-dimethylmorpholine-4-carbonyl)-7-methoxy-4,5-dihydrobenzo[g]indazol-8-yl]pyridine-3-carboxamide (15.82 mg, 25.05 μmol, 29.83% yield, 100% purity, TFA)was obtained as a off-white solid. LCMS (ESI): m/z[M+H] calcd for C29H35N5O4: 517.27; found: 518.3. 1H NMR: (400 MHz, METHANOL-d4)δ=9.05 (br s, 1H), 8.98 (br s, 1H), 8.69-8.63 (m, 1H), 7.63 (s, 1H), 7.24 (s, 1H), 4.48 (t, J=7.3 Hz, 2H), 3.93 (s, 3H), 3.83-3.77 (m, 2H), 3.71-3.65 (m, 2H), 3.50 (s, 2H), 3.04-2.97 (m, 2H), 2.80-2.73 (m, 2H), 1.86 (quin, J=7.4 Hz, 2H), 1.52 (s, 6H), 1.36 (sxt, J=7.4 Hz, 2H), 0.91 (t, J=7.3 Hz, 3H). FIG. 149 shows the nuclear magnetic resonance of Compound 9-15.

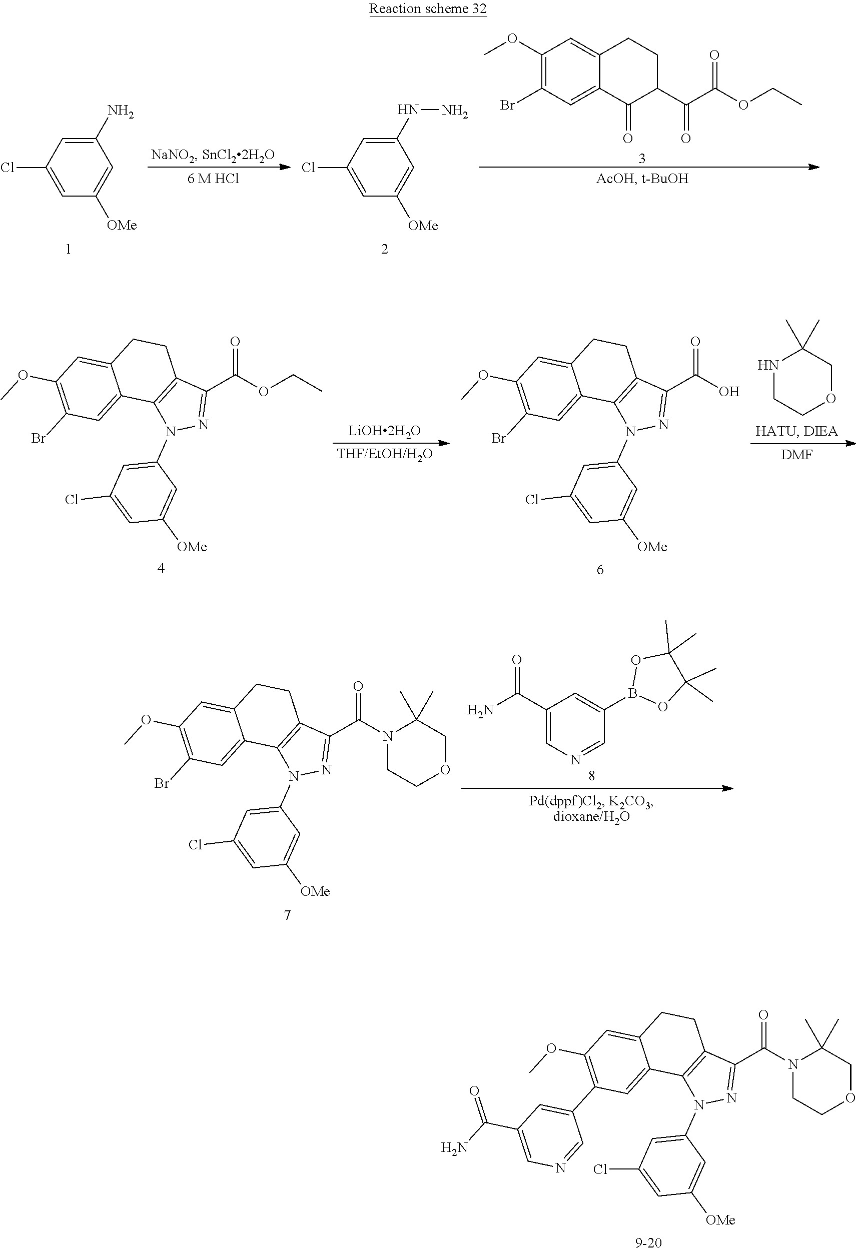

### Synthesis of (3-chloro-5-methoxy-phenyl)hydrazine

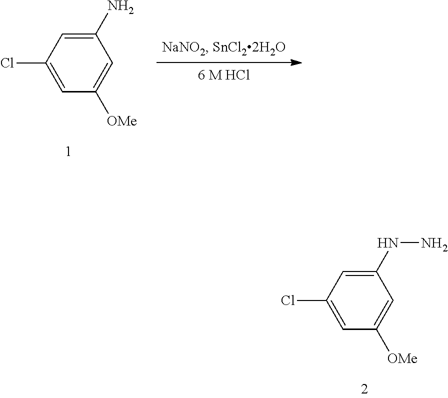 [SMILES: COc1cc(N)cc(Cl)c1 ; COc1cc(Cl)cc(NN)c1]

To a solution of 3-chloro-5-methoxy-aniline (1 g, 6.35 mmol, 1 eq) in HCl (6 M, 10 mL, 9.46 eq) was added NaNO2 (437.83 mg, 6.35 mmol, 2 mL, 1 eq)at 0° C. The mixture was stirred at 0° C. for 1 hr. A solution of SnCl2·2H2O (2.86 g, 12.69 mmol, 2 eq) in HCl (6 M, 10 mL, 9.46 eq) was added slowly at 0° C. Then reaction mixture was stirred at 0° C. for 2 hr. The reaction mixture filtered to give the filter cake. The filter cake was collected and dried in vacuum. Compound (3-chloro-5-methoxy-phenyl)hydrazine (1.33 g, crude, HCl)was obtained as a brown solid. 1H NMR (400 MHz, DMSO-d6)δ=10.25 (br s, 3H), 8.59-8.38 (m, 1H), 6.61 (d, J=1.8 Hz, 1H), 6.59 (t, J=1.9 Hz, 1H), 6.53 (d, J=1.6 Hz, 1H), 3.74 (s, 3H).

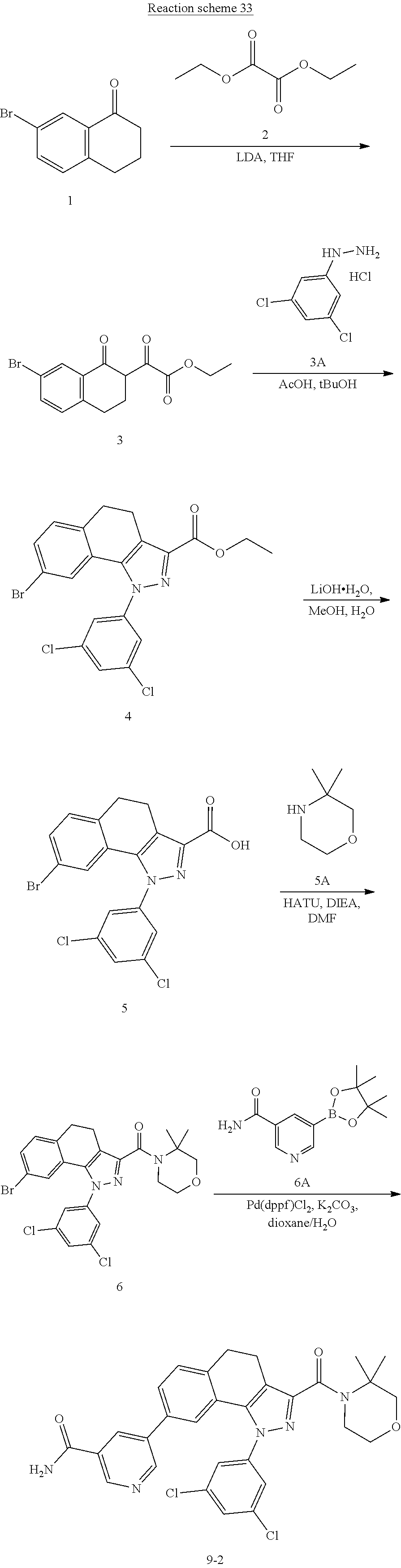

### Synthesis of Compound 9-2

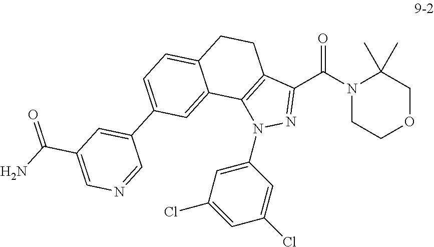 [SMILES: CC1(C)COCCN1C(=O)c1nn(-c2cc(Cl)cc(Cl)c2)c2c1CCc1ccc(-c3cncc(C(N)=O)c3)cc1-2]

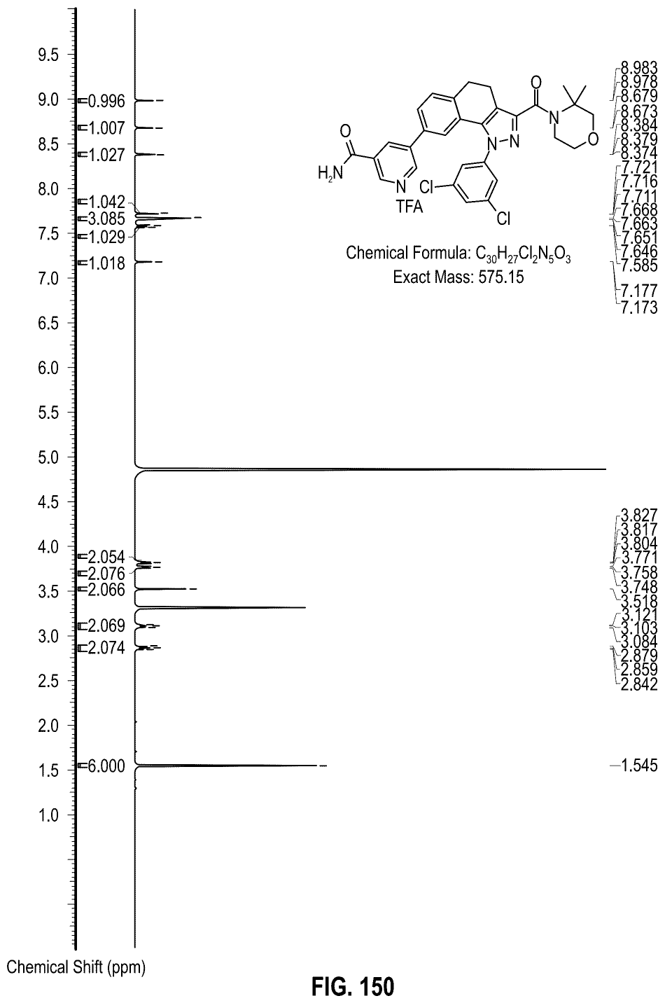

Compound 9-02 was synthesized according to a procedure similar to Reaction scheme 33. LCMS (ESI): m z[M+H] calcd for C₃₀H₂₈Cl₂N₅O₃: 576.15; found: 576.3. ¹H NMR (400 MHz, METHANOL-d4) δ=8.98 (d, J=2.0 Hz, 1H), 8.68 (d, J=2.0 Hz, 1H), 8.38 (t, J=2.0 Hz, 1H), 7.74-7.70 (m, 1H), 7.68-7.64 (m, 3H), 7.59-7.56 (m, 1H), 7.18 (d, J=1.6 Hz, 1H), 3.84-3.79 (m, 2H), 3.78-3.73 (m, 2H), 3.52 (s, 2H), 3.14-3.07 (m, 2H), 2.89-2.83 (m, 2H), 1.54 (s, 6H). FIG. 150 shows the nuclear magnetic resonance of Compound 9-2.

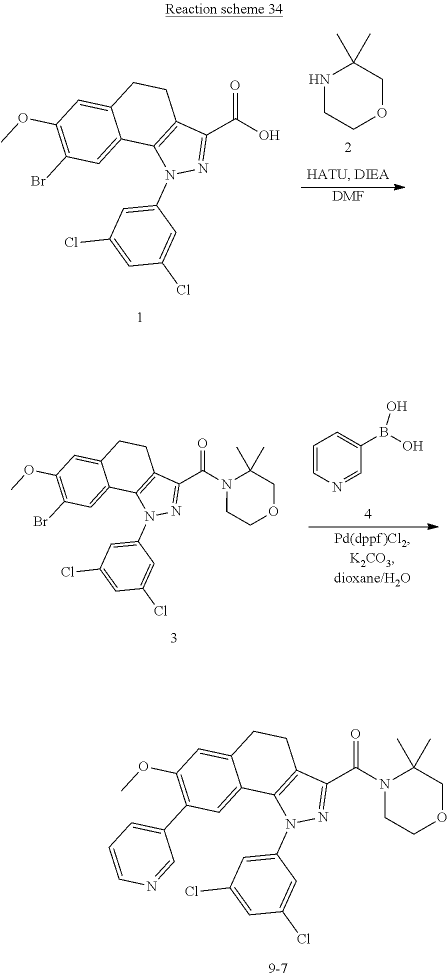

### Synthesis of Compound 9-7

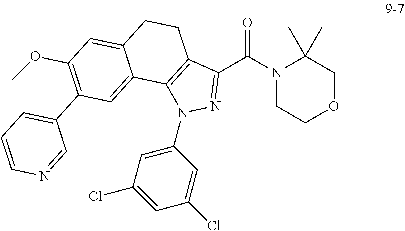 [SMILES: COc1cc2c(cc1-c1cccnc1)-c1c(c(C(=O)N3CCOCC3(C)C)nn1-c1cc(Cl)cc(Cl)c1)CC2]

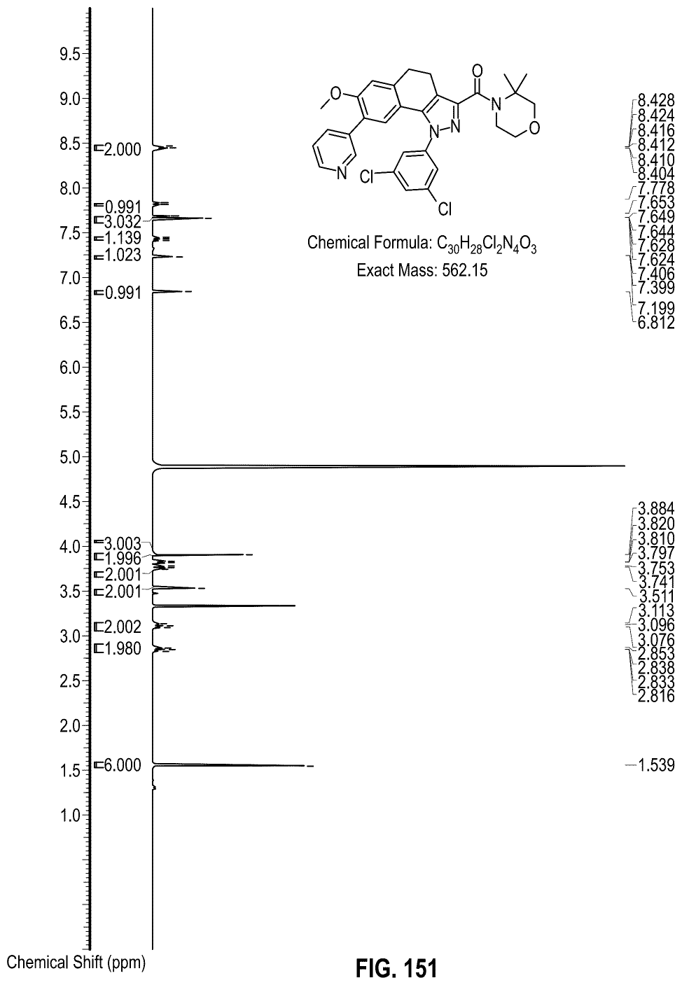

Compound 9-07 was synthesized according to a procedure similar to Reaction scheme 33. LCMS (ESI): m/z [M+H] calcd for C30H29Cl2N4O3: 563.15; found: 563.3. 1H NMR (400 MHz, METHANOL-d4) δ=8.45-8.38 (m, 2H), 7.79 (td, J=2.0, 8.0 Hz, 1H), 7.68-7.60 (m, 3H), 7.40 (ddd, J=0.8, 4.8, 8.0 Hz, 1H), 7.20 (s, 1H), 6.81 (s, 1H), 3.88 (s, 3H), 3.83-3.78 (m, 2H), 3.77-3.71 (m, 2H), 3.51 (s, 2H), 3.13-3.04 (m, 2H), 2.88-2.79 (m, 2H), 1.54 (s, 6H). FIG. 151 shows the nuclear magnetic resonance of Compound 9-7.

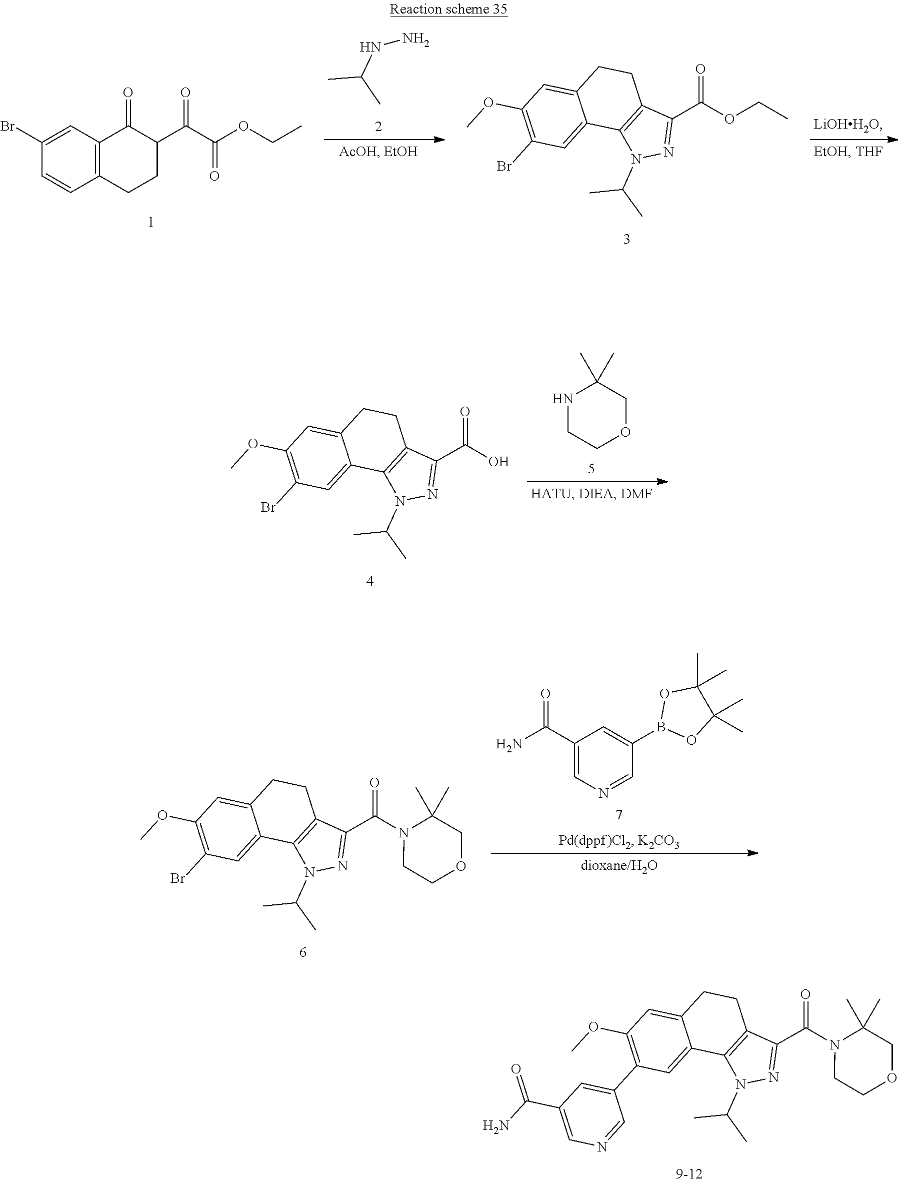

### Synthesis of Compound 9-12

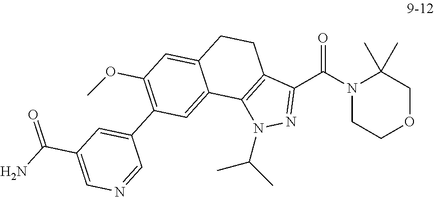 [SMILES: COc1cc2c(cc1-c1cncc(C(N)=O)c1)-c1c(c(C(=O)N3CCOCC3(C)C)nn1C(C)C)CC2]

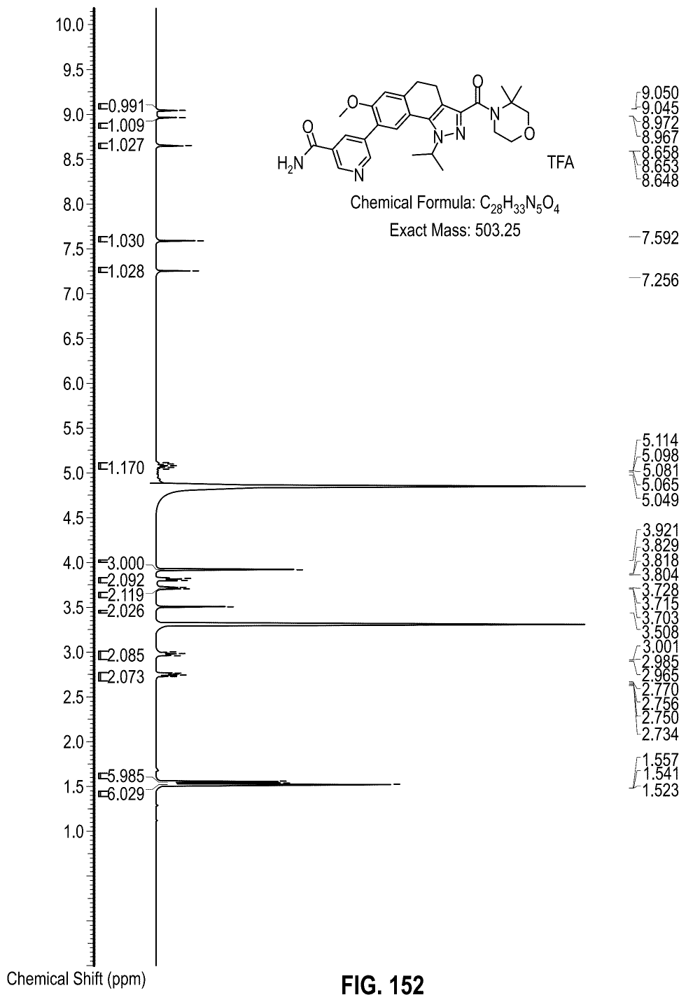

Compound 9-12 was synthesized according to a procedure similar to Reaction scheme 35. LCMS (ESI): m/z [M+H] calcd for C₃₀H₃₅N₅O₆F₃: 504.25; found: 504.3. ¹H NMR (400 MHz, METHANOL-d₄)δ=9.05 (d, J=1.9 Hz, 1H), 8.97 (d, J=2.0 Hz, 1H), 8.65 (t, J=1.9 Hz, 1H), 7.59 (s, 1H), 7.26 (s, 1H), 5.08 (td, J=6.6, 13.1 Hz, 1H), 3.92 (s, 3H), 3.85-3.79 (m, 2H), 3.75-3.69 (m, 2H), 3.51 (s, 2H), 3.02-2.94 (m, 2H), 2.79-2.72 (m, 2H), 1.55 (d, J=6.5 Hz, 6H), 1.52 (s, 6H). FIG. 152 shows the nuclear magnetic resonance of Compound 9-12.

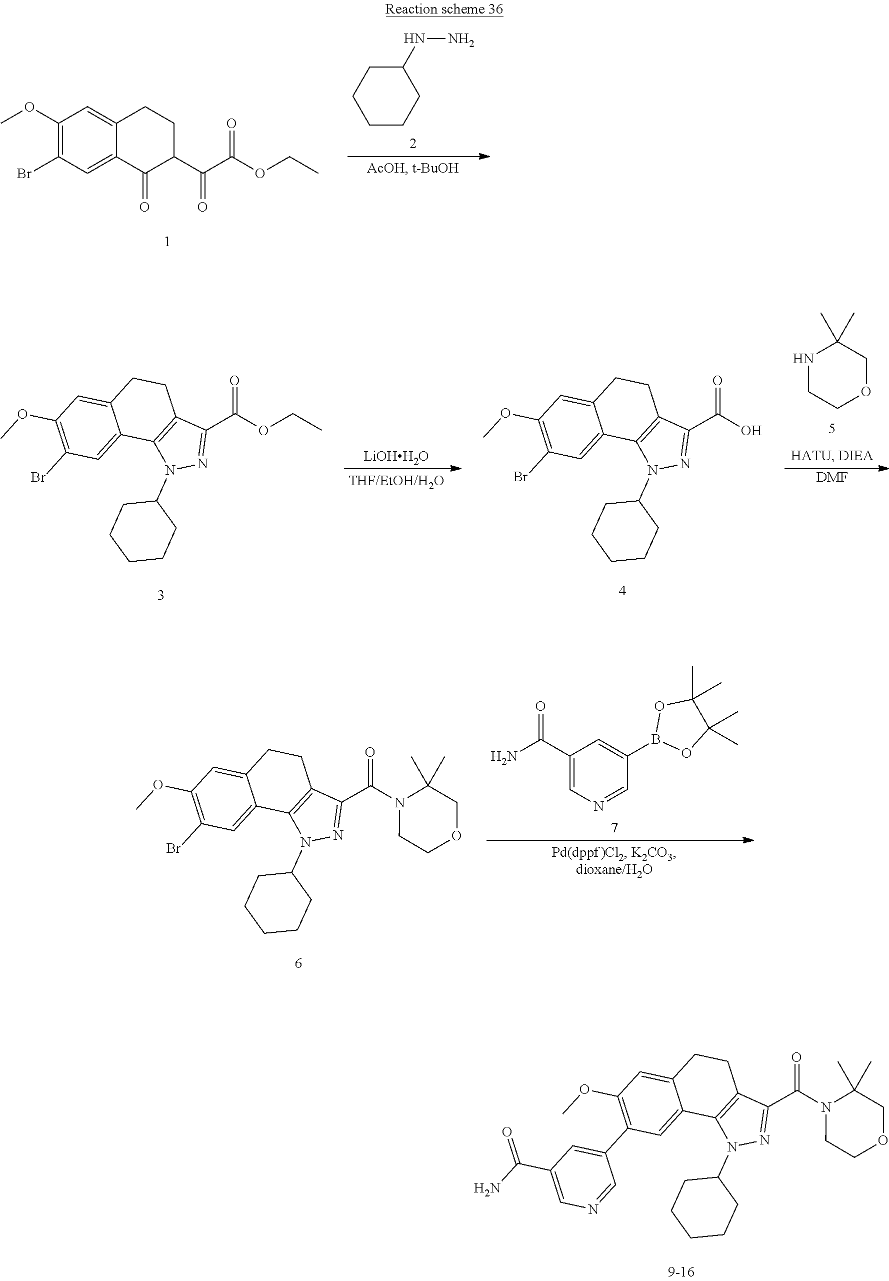

### Synthesis of Compound 9-16

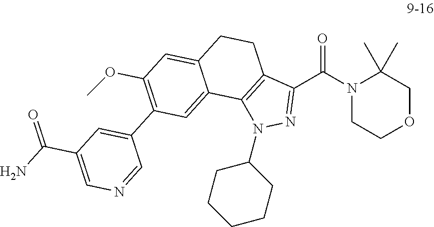 [SMILES: COc1cc2c(cc1-c1cncc(C(N)=O)c1)-c1c(c(C(=O)N3CCOCC3(C)C)nn1C1CCCCC1)CC2]

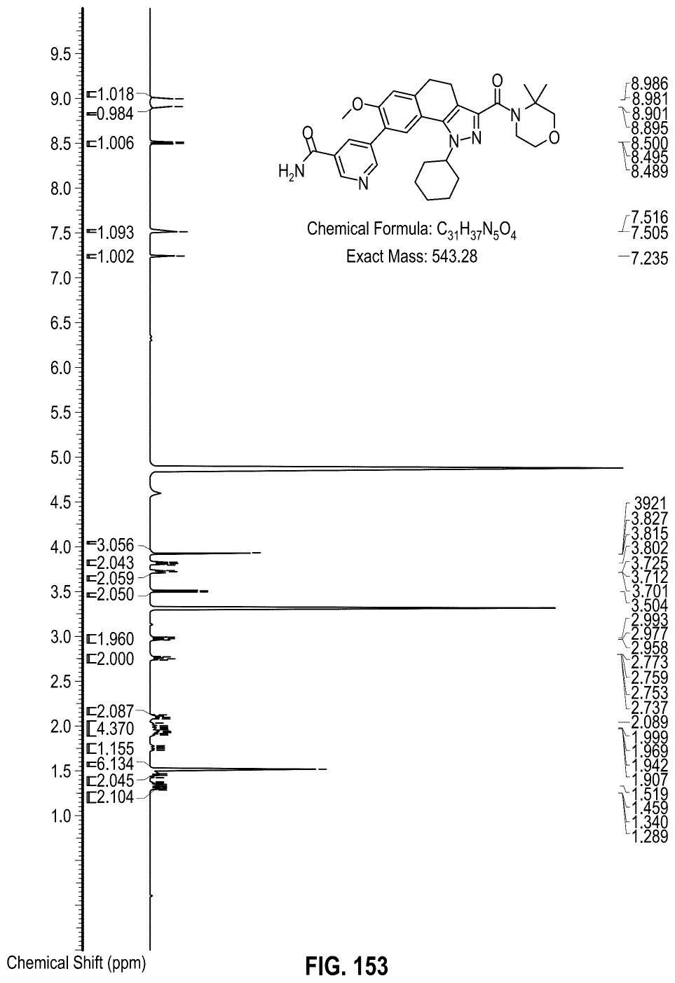

Compound 9-16 was synthesized according to a procedure similar to Reaction scheme 36. LCMS (ESI): m/z [M+H] calcd for C31H38N5O4: 544.28; found: 544.4. 1H NMR (400 MHz, METHANOL-d4)δ=8.98 (d, J=2.0 Hz, 1H), 8.90 (d, J=2.0 Hz, 1H), 8.49 (t, J=2.0 Hz, 1H), 7.50 (s, 1H), 7.23 (s, 1H), 3.92 (s, 3H), 3.84-3.78 (m, 2H), 3.74-3.68 (m, 2H), 3.50 (s, 2H), 3.00-2.95 (m, 2H), 2.79-2.72 (m, 2H), 2.14-2.07 (m, 2H), 2.05-1.88 (m, 4H), 1.80-1.70 (m, 1H), 1.52 (s, 6H), 1.50-1.41 (m, 2H), 1.39-1.27 (m, 2H). FIG. 153 shows the nuclear magnetic resonance of Compound 9-16.

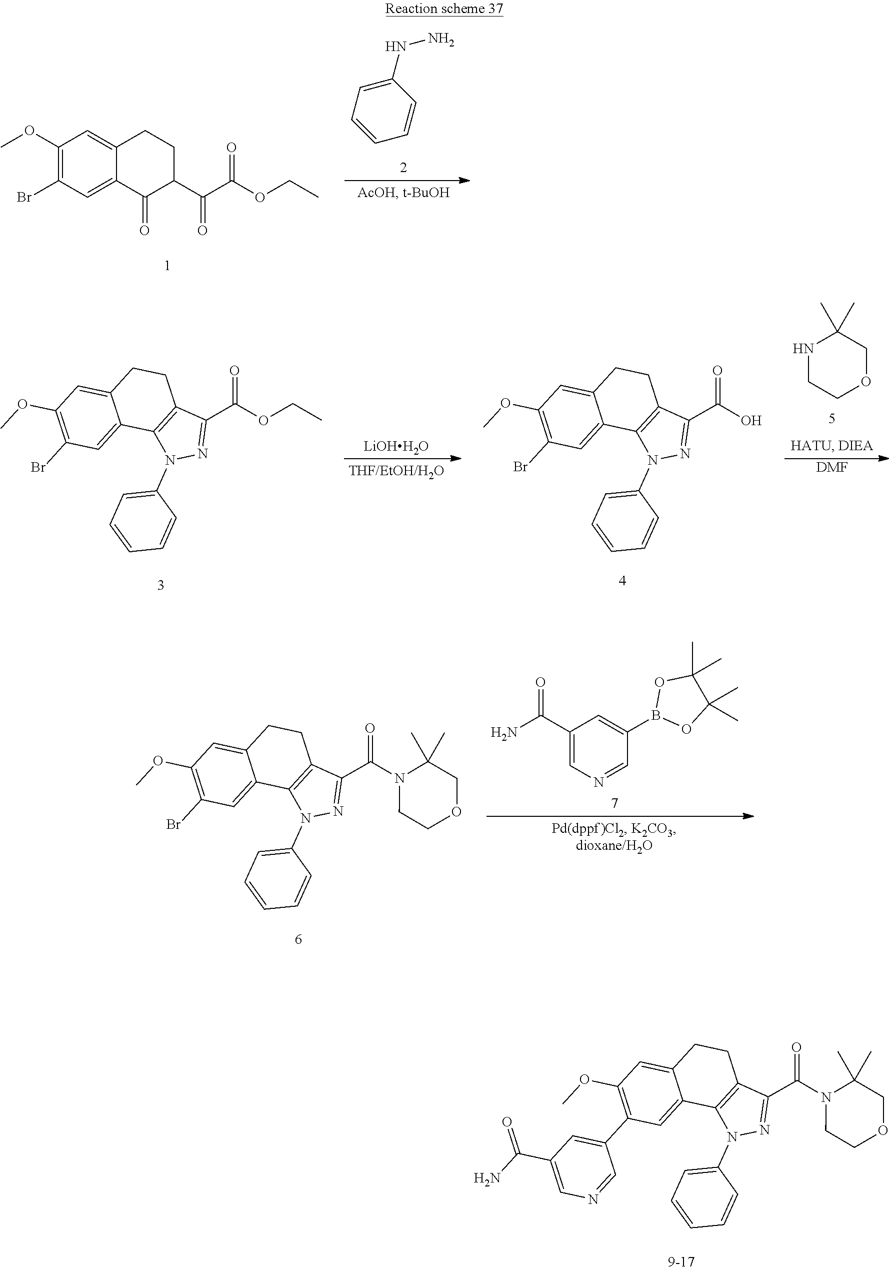

### Synthesis of Compound 9-17

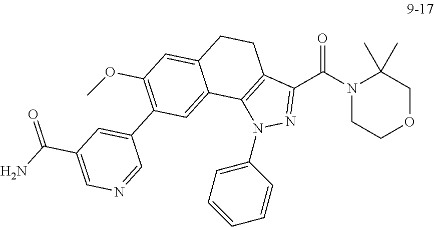 [SMILES: COc1cc2c(cc1-c1cncc(C(N)=O)c1)-c1c(c(C(=O)N3CCOCC3(C)C)nn1-c1ccccc1)CC2]

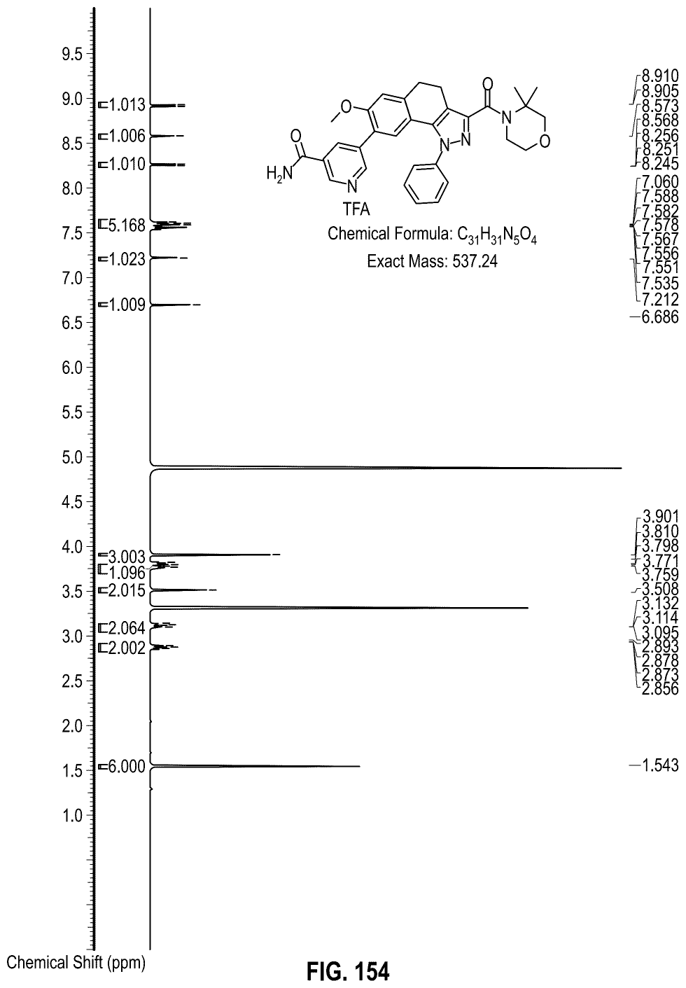

Compound 9-17 was synthesized according to a procedure similar to Reaction scheme 37. LCMS (ESI): m/z [M+H] calcd for C31H32N5O4: 538.24; found: 538.4. 1H NMR (400 MHz, METHANOL-d4)δ=8.91 (d, J=2.0 Hz, 1H), 8.57 (d, J=2.0 Hz, 1H), 8.25 (t, J=2.0 Hz, 1H), 7.63-7.52 (m, 5H), 7.21 (s, 1H), 6.69 (s, 1H), 3.90 (s, 3H), 3.84-3.73 (m, 4H), 3.51 (s, 2H), 3.11 (t, J=7.6 Hz, 2H), 2.91-2.84 (m, 2H), 1.54 (s, 6H). FIG. 154 shows the nuclear magnetic resonance of Compound 9-17.

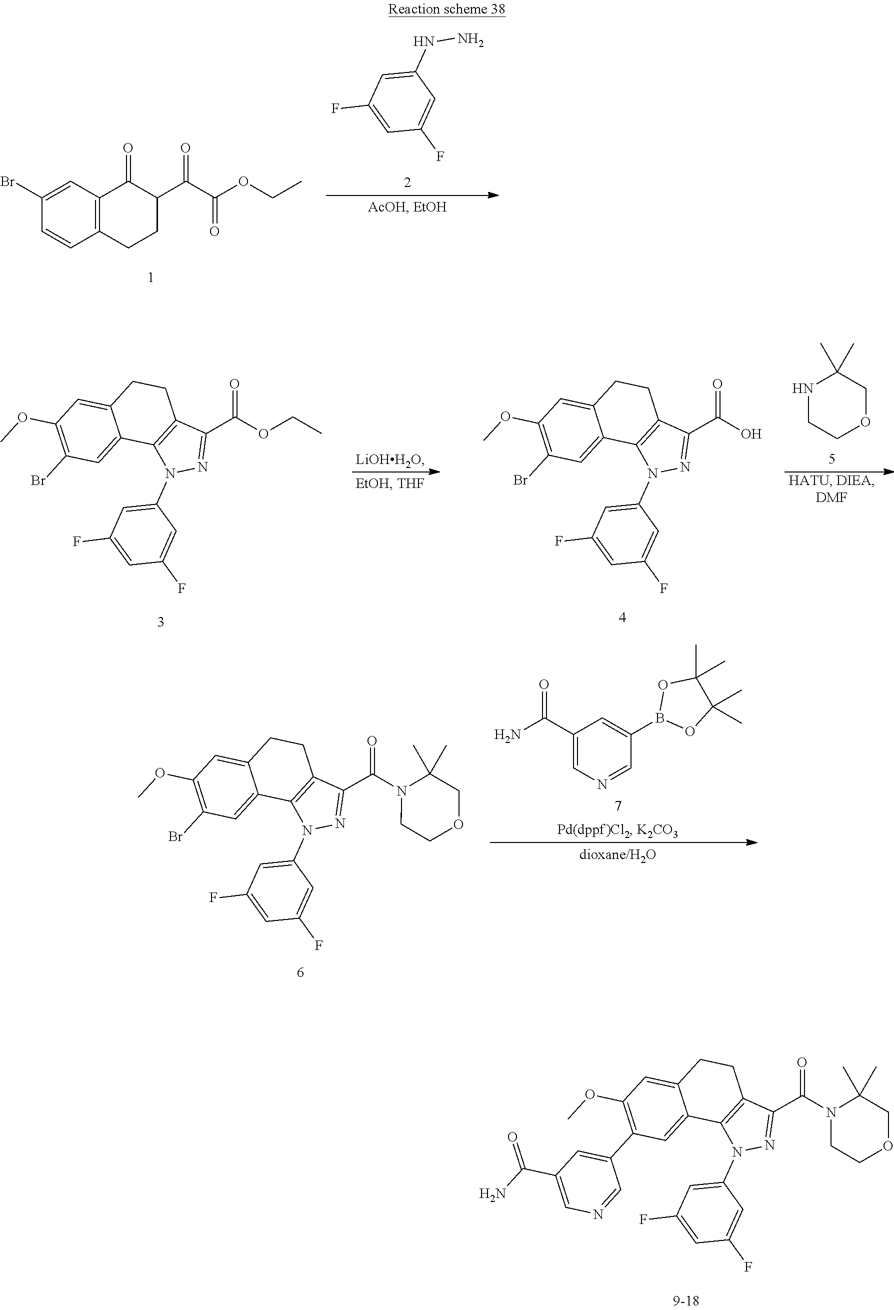

### Synthesis of Compound 9-18

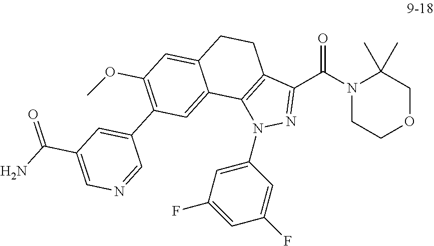 [SMILES: COc1cc2c(cc1-c1cncc(C(N)=O)c1)-c1c(c(C(=O)N3CCOCC3(C)C)nn1-c1cc(F)cc(F)c1)CC2]

Compound 9-18 was synthesized according to a procedure similar to Reaction scheme 38. LCMS (ESI): m/z [M+H] calcd for C33H31F5N5O6: 574.22; found: 574.3. 1H NMR (400 MHz, METHANOL-d4)δ=8.96 (d, J=1.6 Hz, 1H), 8.68 (d, J=1.4 Hz, 1H), 8.38 (s, 1H), 7.31-7.23 (m, 3H), 7.22-7.13 (m, 1H), 6.91 (s, 1H), 3.92 (s, 3H), 3.83-3.79 (m, 2H), 3.78-3.73 (m, 2H), 3.51 (s, 2H), 3.14-3.08 (m, 2H), 2.85 (t, J=7.3 Hz, 2H), 1.54 (s, 6H). FIG. 154 shows the nuclear magnetic resonance of Compound 9-18.

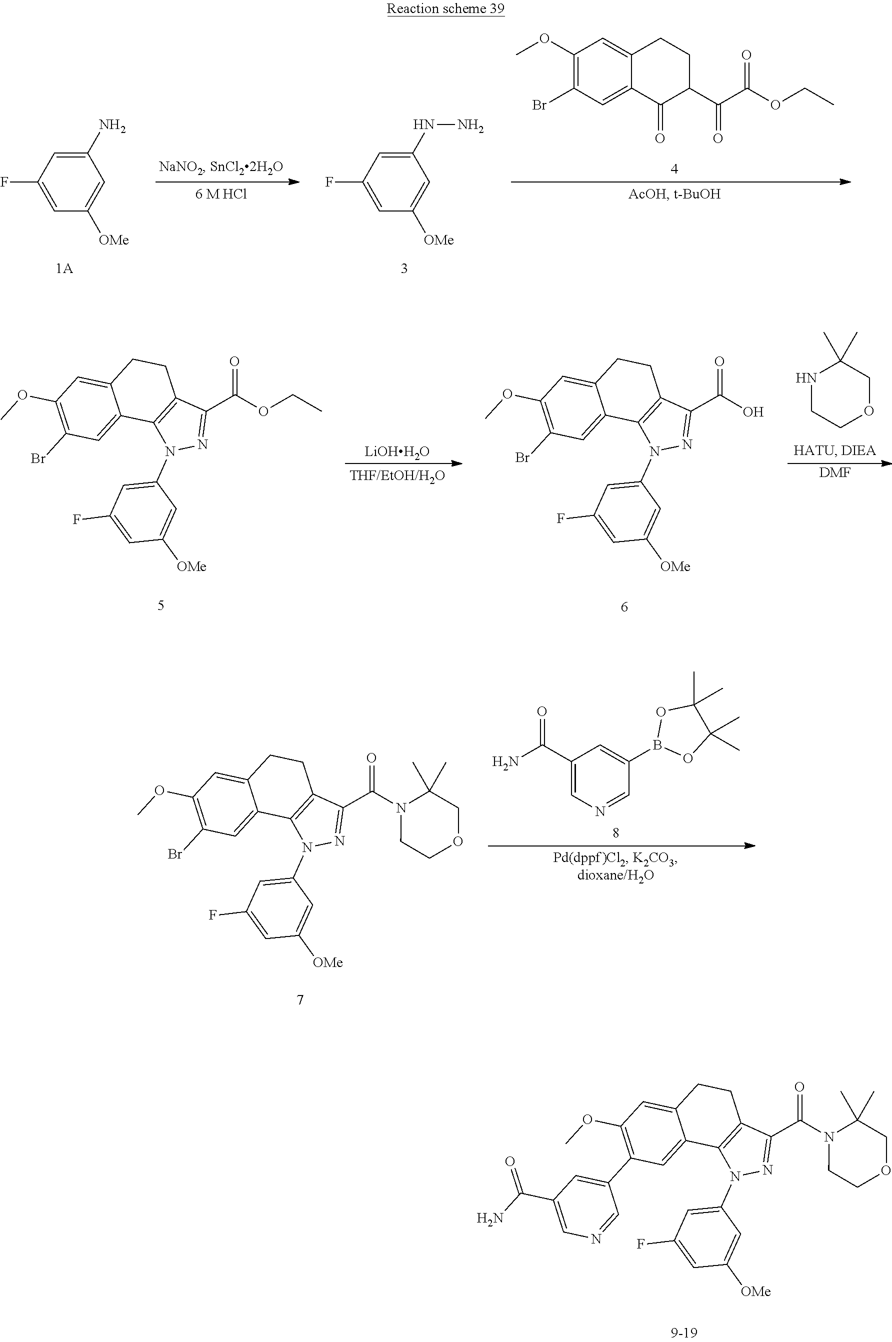

### Synthesis of Compound 9-19

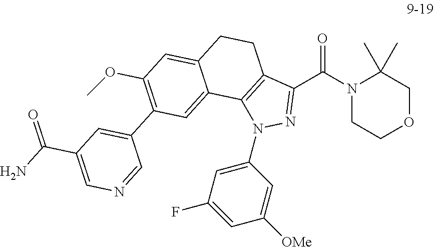 [SMILES: COc1cc(F)cc(-n2nc(C(=O)N3CCOCC3(C)C)c3c2-c2cc(-c4cncc(C(N)=O)c4)c(OC)cc2CC3)c1]

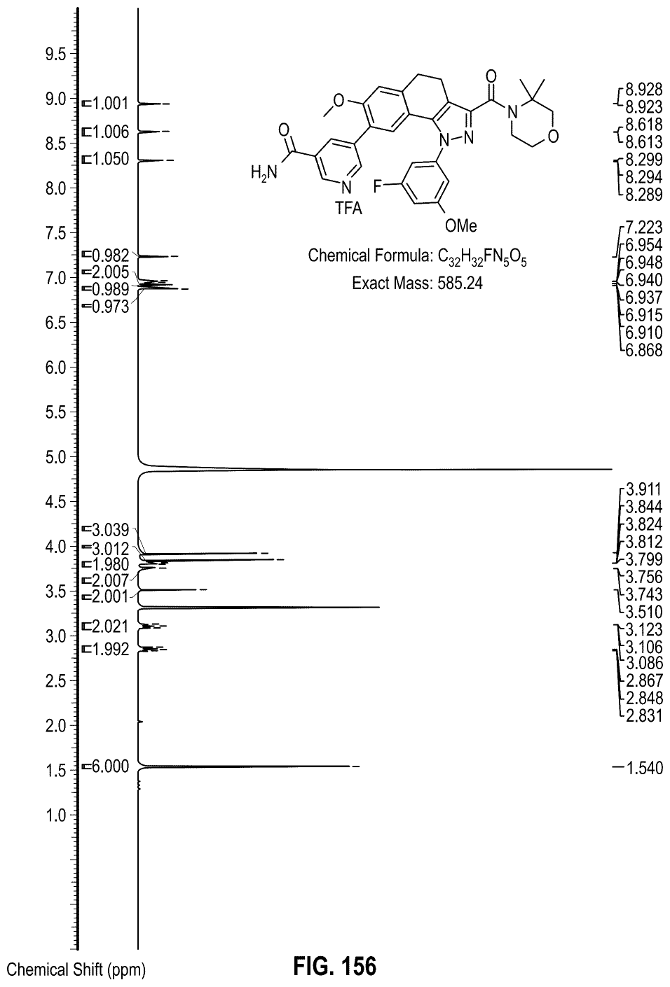

Compound 9-19 was synthesized according to a procedure similar to Reaction scheme 38. LCMS (ESI): m/z[M+H] calcd for C₃₂H₃₃FN₅O₅: 586.24; found: 586.4. ¹H NMR (400 MHz, METHANOL-d4)δ=8.93 (d, J=2.0 Hz, 1H), 8.62 (d, J=2.0 Hz, 1H), 8.29 (t, J=2.0 Hz, 1H), 7.22 (s, 1H), 6.94 (dd, J=1.8, 4.9 Hz, 2H), 6.91 (d, J=2.0 Hz, 1H), 6.87 (s, 1H), 3.91 (s, 3H), 3.84 (s, 3H), 3.83-3.79 (m, 2H), 3.77-3.72 (m, 2H), 3.51 (s, 2H), 3.11 (t, J=7.4 Hz, 2H), 2.89-2.81 (m, 2H), 1.54 (s, 6H). FIG. 156 shows the nuclear magnetic resonance of Compound 9-19.

### Synthesis of Compound 9-20

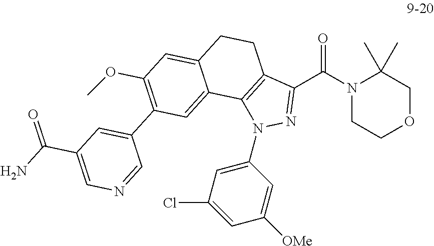 [SMILES: COc1cc(Cl)cc(-n2nc(C(=O)N3CCOCC3(C)C)c3c2-c2cc(-c4cncc(C(N)=O)c4)c(OC)cc2CC3)c1]

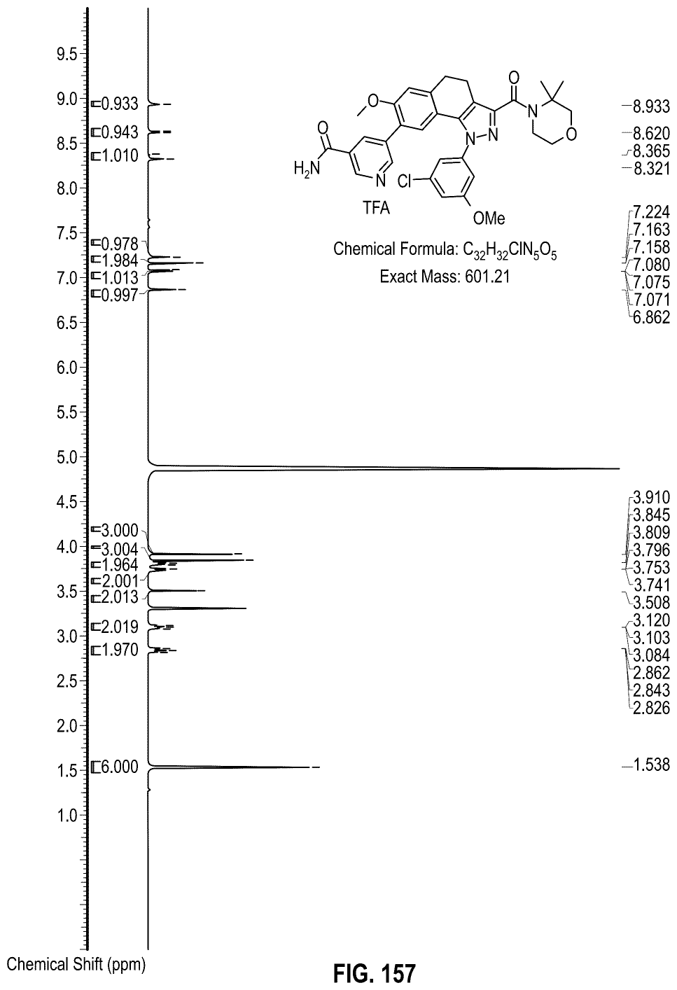

Compound 9-20 was synthesized according to a procedure similar to Reaction scheme 39. LCMS (ESI): m/z[M+H] calcd for C32H33ClN5O5: 602.21; found: 302.3. 1H NMR (400 MHz, METHANOL-d4)δ=8.93 (s, 1H), 8.62 (s, 1H), 8.32 (s, 1H), 7.22 (s, 1H), 7.16 (d, J=1.9 Hz, 2H), 7.08 (t, J=1.9 Hz, 1H), 6.86 (s, 1H), 3.91 (s, 3H), 3.85 (s, 3H), 3.80 (br d, J=5.3 Hz, 2H), 3.75 (br d, J=4.9 Hz, 2H), 3.51 (s, 2H), 3.10 (br t, J=7.3 Hz, 2H), 2.89-2.80 (m, 2H), 1.54 (s, 6H). FIG. 157 shows the nuclear magnetic resonance of Compound 9-20.

### Example 10

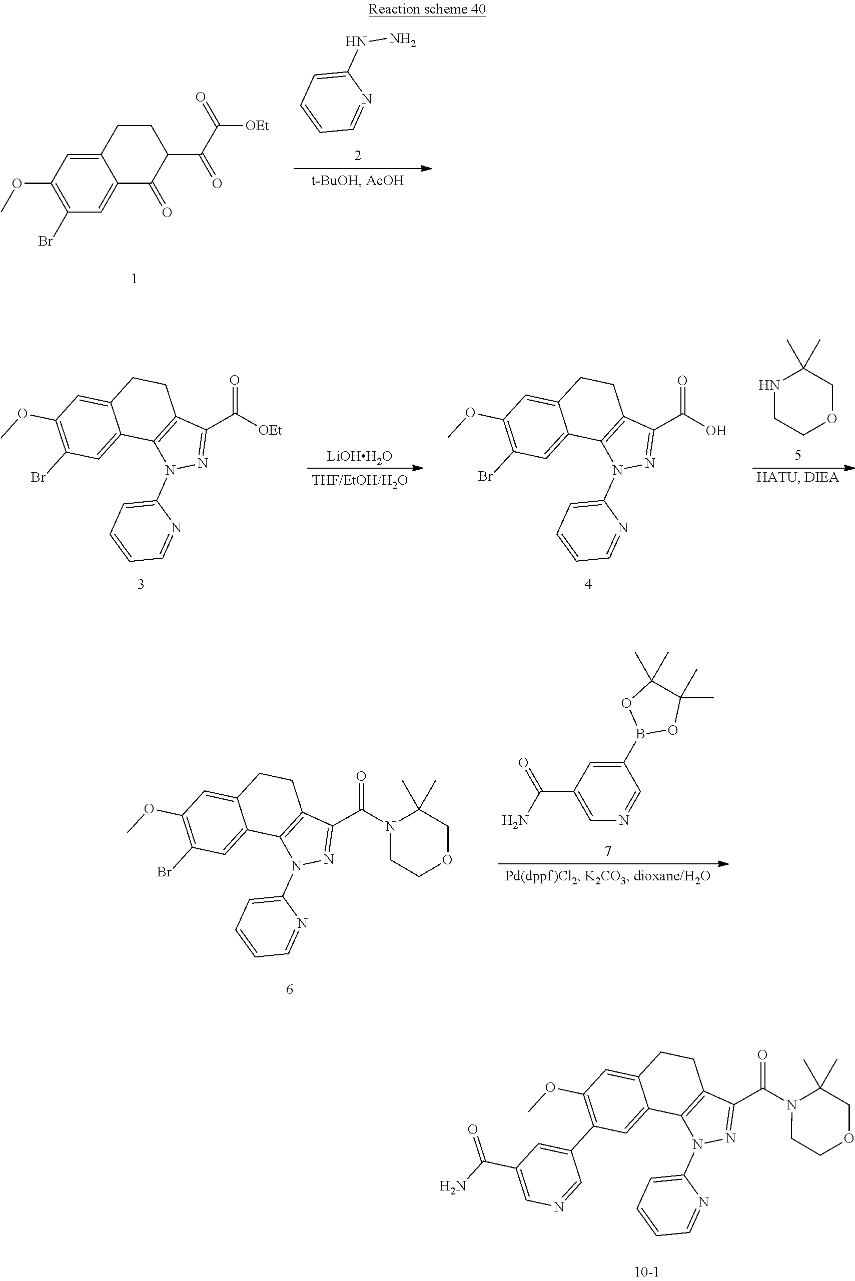

### Synthesis of ethyl 8-bromo-7-methoxy-1-(2-pyridyl)-4,5-dihydrobenzo[g]indazole-3-carboxylate

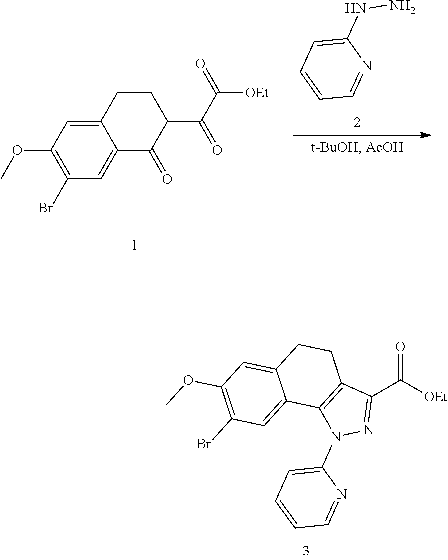 [SMILES: CCOC(=O)C(=O)C1CCc2cc(OC)c(Br)cc2C1=O ; NNc1ccccn1 ; CCOC(=O)c1nn(-c2ccccn2)c2c1CCc1cc(OC)c(Br)cc1-2]

To a solution of ethyl2-(7-bromo-6-methoxy-1-oxo-tetralin-2-yl)-2-oxo-acetate (200 mg, 563.09 μmol, 1 eq) in t-BuOH (5 mL) was added AcOH (169.07 mg, 2.82 mmol, 161.18 μL, 5 eq) and 2-pyridylhydrazine (61.45 mg, 563.09 μmol, 1 eq) in one portion. The mixture was stirred at 100° C. for 5 hours. The reaction mixture was filtered and the filtrate was concentrated under reduced pressure to give a residue. The residue was purified by prep-TLC (SiO2, Petroleum ether/Ethyl acetate=2/1) to afford ethyl 8-bromo-7-methoxy-1-(2-pyridyl)-4,5-dihydrobenzo[g]indazole-3-carboxylate (56 mg, 130.76 μmol, 23.22% yield) as a yellow solid. 1H NMR (400 MHz, CHLOROFORM-d) δ=8.55 (td, J=0.9, 4.8 Hz, 1H), 8.01-7.93 (m, 1H), 7.85 (d, J=8.0 Hz, 1H), 7.44 (ddd, J=1.1, 4.9, 7.4 Hz, 1H), 7.10 (s, 1H), 6.86 (s, 1H), 4.46 (q, J=7.1 Hz, 2H), 3.92 (s, 3H), 3.12-3.04 (m, 2H), 3.03-2.95 (m, 2H), 1.44 (t, J=7.1 Hz, 3H).

### Synthesis of 8-bromo-7-methoxy-1-(2-pyridyl)-4,5-dihydrobenzo[g]indazole-3-carboxylic acid

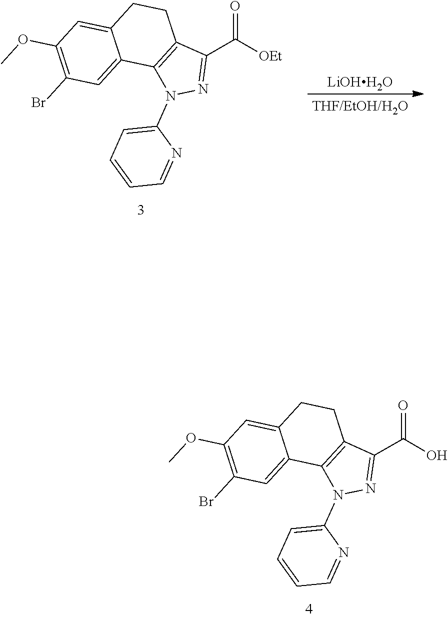 [SMILES: CCOC(=O)c1nn(-c2ccccn2)c2c1CCc1cc(OC)c(Br)cc1-2 ; "LiOH·H2O" ; COc1cc2c(cc1Br)-c1c(c(C(=O)O)nn1-c1ccccn1)CC2]

To a solution of ethyl 8-bromo-7-methoxy-1-(2-pyridyl)-4,5-dihydrobenzo[g]indazole-3-carboxylate (56 mg, 130.76 μmol, 1 eq) in THF (2 mL) and H2O (2 mL) was added LiOH·H2O (16.46 mg, 392.27 μmol, 3 eq) and EtOH (1 mL) in one portion. The mixture was stirred at 20° C. for 2 hours. The mixture was concentrated under reduced pressure to give a residue. The residue was diluted with water (10 mL). The pH of the reaction mixture was adjusted to 2-3 with 1 M HCl aqueous solution. The residue was extracted with ethyl acetate (10 mL*2). The combined organic layers were washed with brine (10 mL), dried over anhydrous Na2SO4, filtered and concentrated under reduced pressure to afford 8-bromo-7-methoxy-1-(2-pyridyl)-4,5-dihydrobenzo[g]indazole-3-carboxylic acid (42 mg, 104.94 μmol, 80.26% yield) as a yellow solid.

### Synthesis of [8-bromo-7-methoxy-1-(2-pyridyl)-4,5-dihydrobenzo[g]indazol-3-yl]-(3,3-dimethylmorpholin-4-yl)methanone

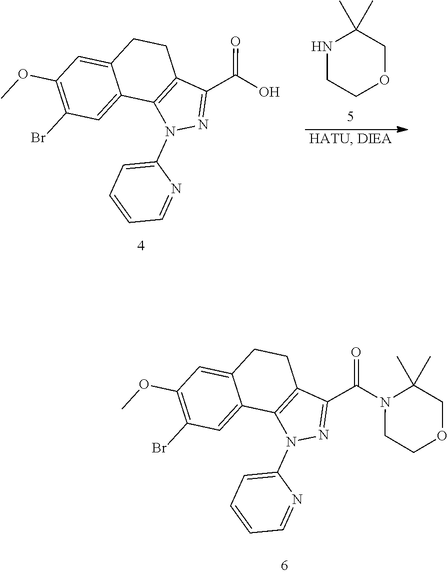

To a solution of 8-bromo-7-methoxy-1-(2-pyridyl)-4,5-dihydrobenzo[g]indazole-3-carboxylic acid (42 mg, 104.94 μmol, 1 eq) in DMF (2 mL) was added HATU (59.85 mg, 157.41 μmol, 1.5 eq) and DIEA (40.69 mg, 314.82 μmol, 54.84 μL, 3 eq) in one portion. The mixture was stirred at 20° C. for 30 min. Then to the mixture was added a mixture of 3,3-dimethylmorpholine (21.76 mg, 188.89 μmol, 1.8 eq) in DMF (0.5 mL). The mixture was stirred at 20° C. for 16 hours. The reaction mixture was diluted with water (5 mL) and filtered. The filter cake was dried in vacuum to afford [8-bromo-7-methoxy-1-(2-pyridyl)-4,5-dihydrobenzo[g]indazol-3-yl]-(3,3-dimethylmorpholin-4-yl)methanone (42 mg, 84.44 μmol, 80.47% yield) as a brown oil.

### Synthesis of Compound 10-1

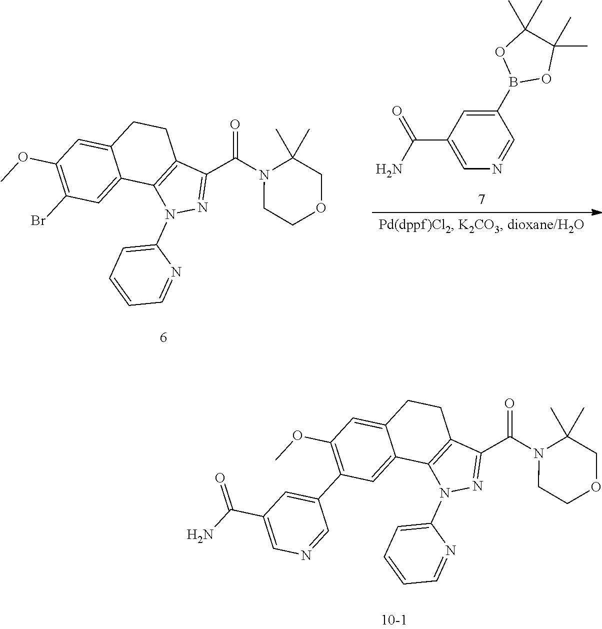 [SMILES: COc1cc2c(cc1Br)-c1c(c(C(=O)N3CCOCC3(C)C)nn1-c1ccccn1)CC2 ; CC1(C)OB(c2cncc(C(N)=O)c2)OC1(C)C ; COc1cc2c(cc1-c1cncc(C(N)=O)c1)-c1c(c(C(=O)N3CCOCC3(C)C)nn1-c1ccccn1)CC2]

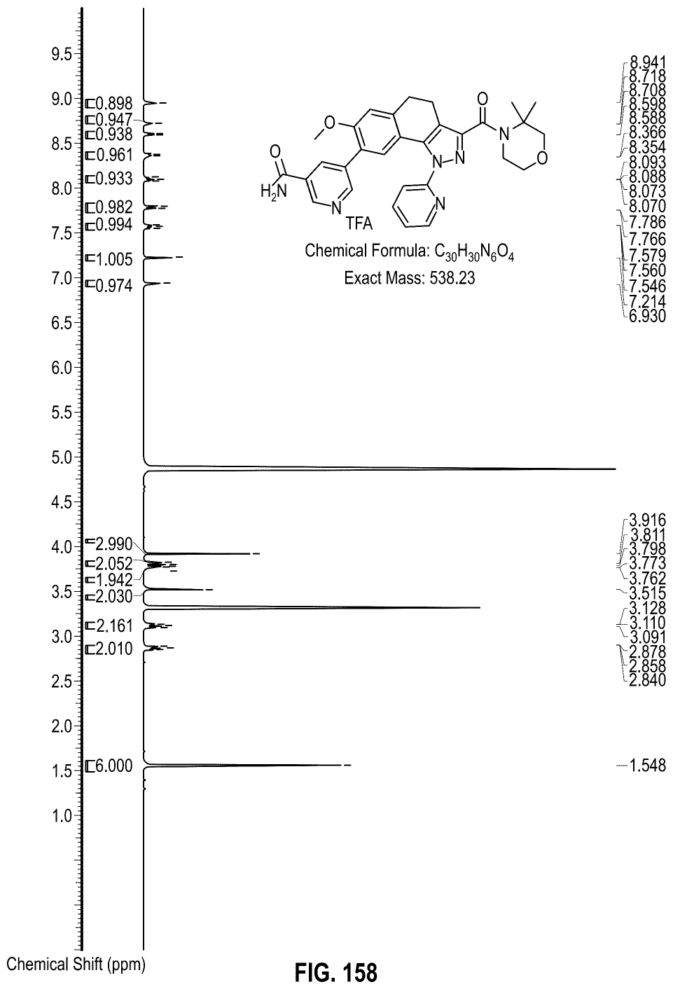

To a solution of [8-bromo-7-methoxy-1-(2-pyridyl)-4,5-dihydrobenzo[g]indazol-3-yl]-(3,3-dimethylmorpholin-4-yl)methanone (42 mg, 84.44 μmol, 1 eq) and 5-(4,4,5,5-tetramethyl-1,3,2-dioxaborolan-2-yl)pyridine-3-carboxamide (31.42 mg, 126.66 μmol, 1.5 eq) in dioxane (2 mL) and H2O (1 mL) was added Pd(dppf)Cl2 (6.18 mg, 8.44 μmol, 0.1 eq) and K2CO3 (23.34 mg, 168.88 μmol, 2 eq) in one portion. The mixture was stirred at 80° C. for 18 hours under N2 atomsphere. The reaction mixture was filtered and the filtrate was concentrated under reduced pressure to give a residue. The residue was purified by prep-HPLC (column: Phenomenex luna C18 150*25 mm*10 um;mobile phase: [water(TFA)-ACN];gradient:28%-58% B over min) followed by lyophilization to afford 5-[3-(3,3-dimethylmorpholine-4-carbonyl)-7-methoxy-1-(2-pyridyl)-4,5-dihydrobenzo[g]indazol-8-yl]pyridine-3-carboxamide (12.18 mg, 17.54 μmol, 20.78% yield, 94% purity, TFA) as a white solid. LCMS (ESI): m/z [M+H] calcd for C30H31N6O4: 539.23; found: 539.3 TH NMR (400 MHz, METHANOL-d4) δ=8.94 (s, 1H), 8.71 (br d, J=3.9 Hz, 1H), 8.59 (br d, J=4.3 Hz, 1H), 8.36 (br d, J=4.8 Hz, 1H), 8.09 (dt, J=1.7, 7.7 Hz, 1H), 7.78 (d, J=7.9 Hz, 1H), 7.56 (dd, J=5.5, 7.5 Hz, 1H), 7.21 (s, 1H), 6.93 (s, 1H), 3.92 (s, 3H), 3.85-3.79 (m, 2H), 3.77 (br d, J=4.8 Hz, 2H), 3.51 (s, 2H), 3.11 (br t, J=7.4 Hz, 2H), 2.91-2.81 (m, 2H), 1.55 (s, 6H). FIG. 158 shows the nuclear magnetic resonance of Compound 10-1.

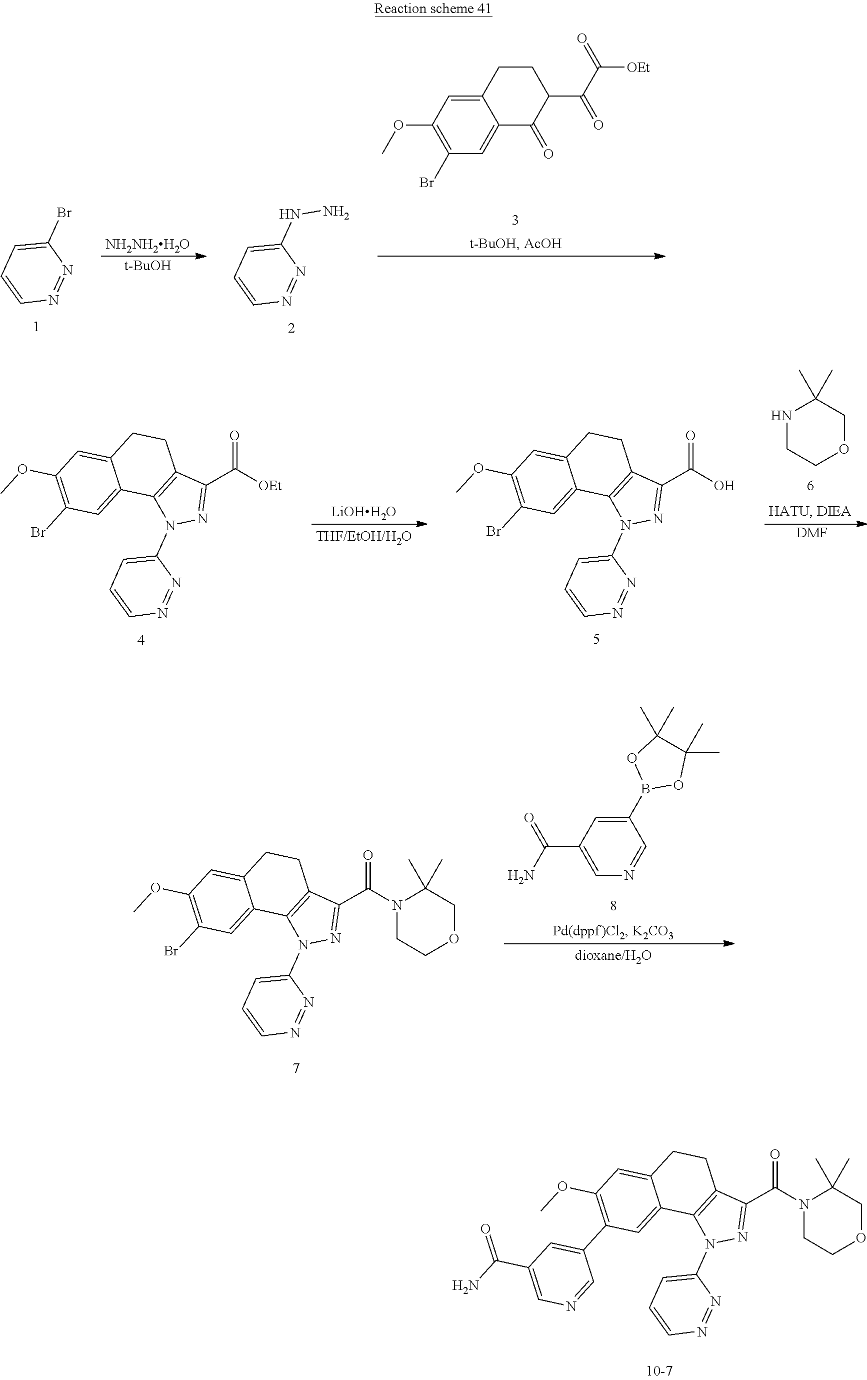

### Synthesis of pyridazin-3-ylhydrazine

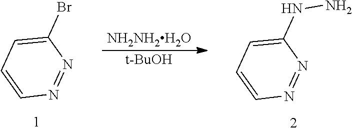

To a solution of 3-bromopyridazine (1 g, 6.29 mmol, 1 eq) in t-BuOH (15 mL) was added NH2NH2·H2O (629.75 mg, 12.58 mmol, 610.22 μL, 2 eq) in one portion. The mixture was stirred at 80° C. for 40 hours. The reaction mixture was diluted with water (30 ml) and concentrated under reduced pressure to afford pyridazin-3-ylhydrazine (900 mg, crude) as a brown solid. 1H NMR (400 MHz, METHANOL-d4) δ=8.41 (d, J=4.3 Hz, 1H), 7.35 (dd, J=4.4, 9.2 Hz, 1H), 7.15 (d, J=9.0 Hz, 1H).

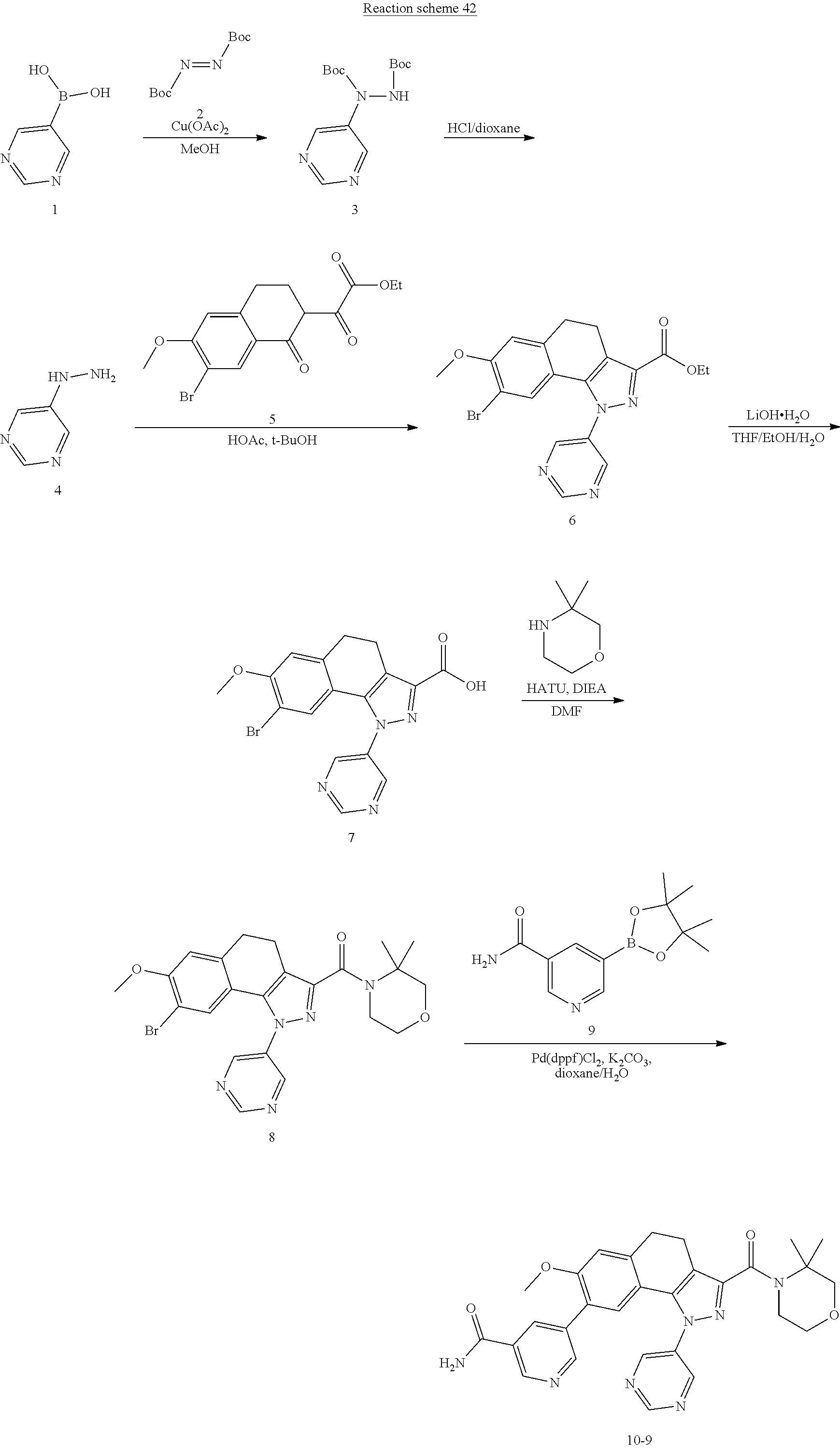

### Synthesis of tert-butyl N-(tert-butoxycarbonylamino)-N-pyrimidin-5-yl-carbamate

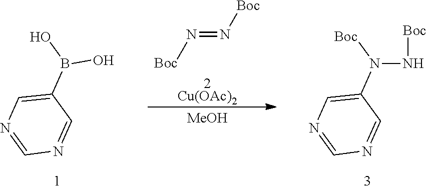

To a solution of pyrimidin-5-ylboronic acid (1 g, 8.07 mmol, 1 eq) in MeOH (15 mL) was added Cu(OAc)2 (73.30 mg, 403.53 μmol, 0.05 eq) and tert-butyl (NE) —N-tert-butoxycarbonyliminocarbamate (1.86 g, 8.07 mmol, 1 eq). The mixture was stirred at 60° C. for 1 hr. The reaction mixture was then concentrated under high vacuum and the resulting mixture was dissolved in EtOAc (50 mL). The organic layer was washed with saturated NaHCO3 (50 mL), water (50 mL), brine (50 mL), dried over anhydrous Na2SO4 and filtered and concentrated under reduced pressure to give a residue. The crude product tert-butyl N-(tert-butoxycarbonylamino) —N-pyrimidin-5-yl-carbamate (2.1 g, crude) as a yellow oil was used into the next step without further purification. 1H NMR (400 MHz, CHLOROFORM-d) δ=9.01-8.84 (m, 3H), 6.77 (br s, 1H), 1.54 (s, 9H), 1.52 (s, 9H).

### Synthesis of pyrimidin-5-ylhydrazine

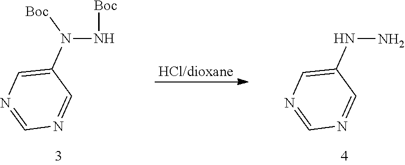 [SMILES: CC(C)(C)OC(=O)NN(C(=O)OC(C)(C)C)c1cncnc1 ; "HCl/dioxane" ; NNc1cncnc1]

A solution of tert-butyl N-(tert-butoxycarbonylamino)-N-pyrimidin-5-yl-carbamate (200 mg, 644.44 μmol, 1 eq) in HCl/dioxane (2 M, 4 mL, 12.41 eq) was stirred at 25° C. for 18 h. The reaction mixture was concentrated under reduced pressure to give a residue. The crude product pyrimidin-5-ylhydrazine (94 mg, crude, HCl) as a yellow solid was used into the next step without further purification.

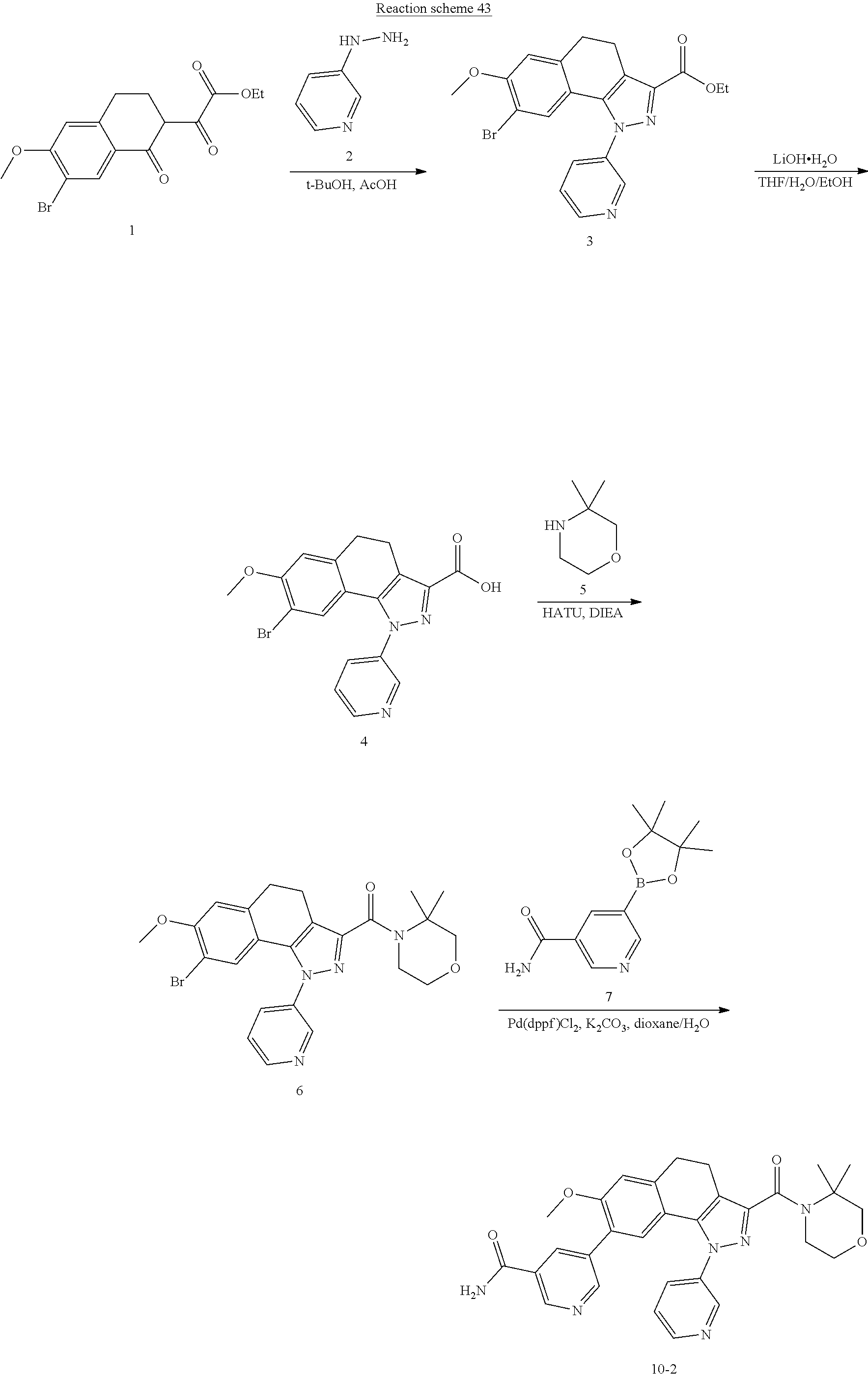

### Synthesis of Compound 10-2

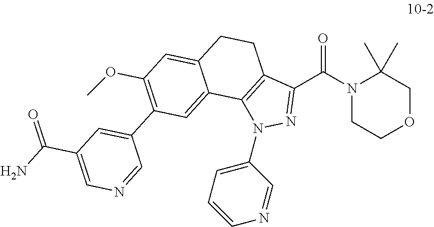 [SMILES: COc1cc2c(cc1-c1cncc(C(N)=O)c1)-c1c(c(C(=O)N3CCOCC3(C)C)nn1-c1cccnc1)CC2]

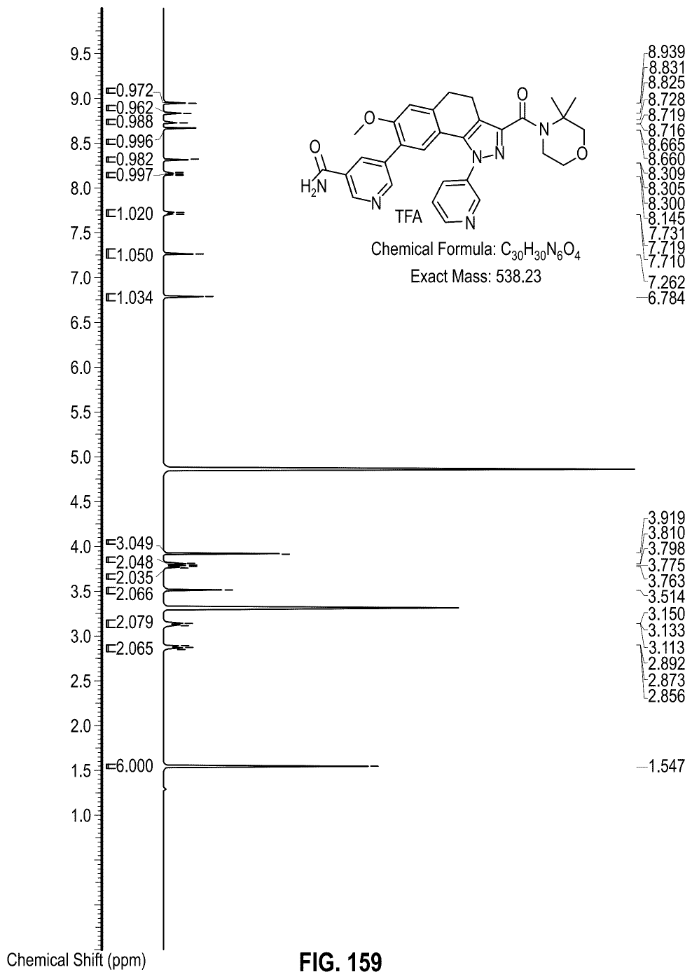

Compound 10-2 was synthesized based on a procedure similar to Reaction scheme 43. LCMS (ESI): m/z [M+H] calcd for C30H31N6O4: 538.23; found: 539.3. 1H NMR (400 MHz, METHANOL-d4) δ=8.94 (d, J=1.8 Hz, 1H), 8.83 (d, J=2.3 Hz, 1H), 8.72 (dd, J=1.2, 4.9 Hz, 1H), 8.66 (d, J=2.0 Hz, 1H), 8.30 (t, J=1.9 Hz, 1H), 8.15 (dd, J=1.8, 8.3 Hz, 1H), 7.71 (dd, J=4.8, 8.3 Hz, 1H), 7.26 (s, 1H), 6.78 (s, 1H), 3.92 (s, 3H), 3.80 (br d, J=4.9 Hz, 2H), 3.77 (br d, J=4.8 Hz, 2H), 3.51 (s, 2H), 3.17-3.09 (m, 2H), 2.91-2.82 (m, 2H), 1.55 (s, 6H). FIG. 159 shows the nuclear magnetic resonance of Compound 10-2.

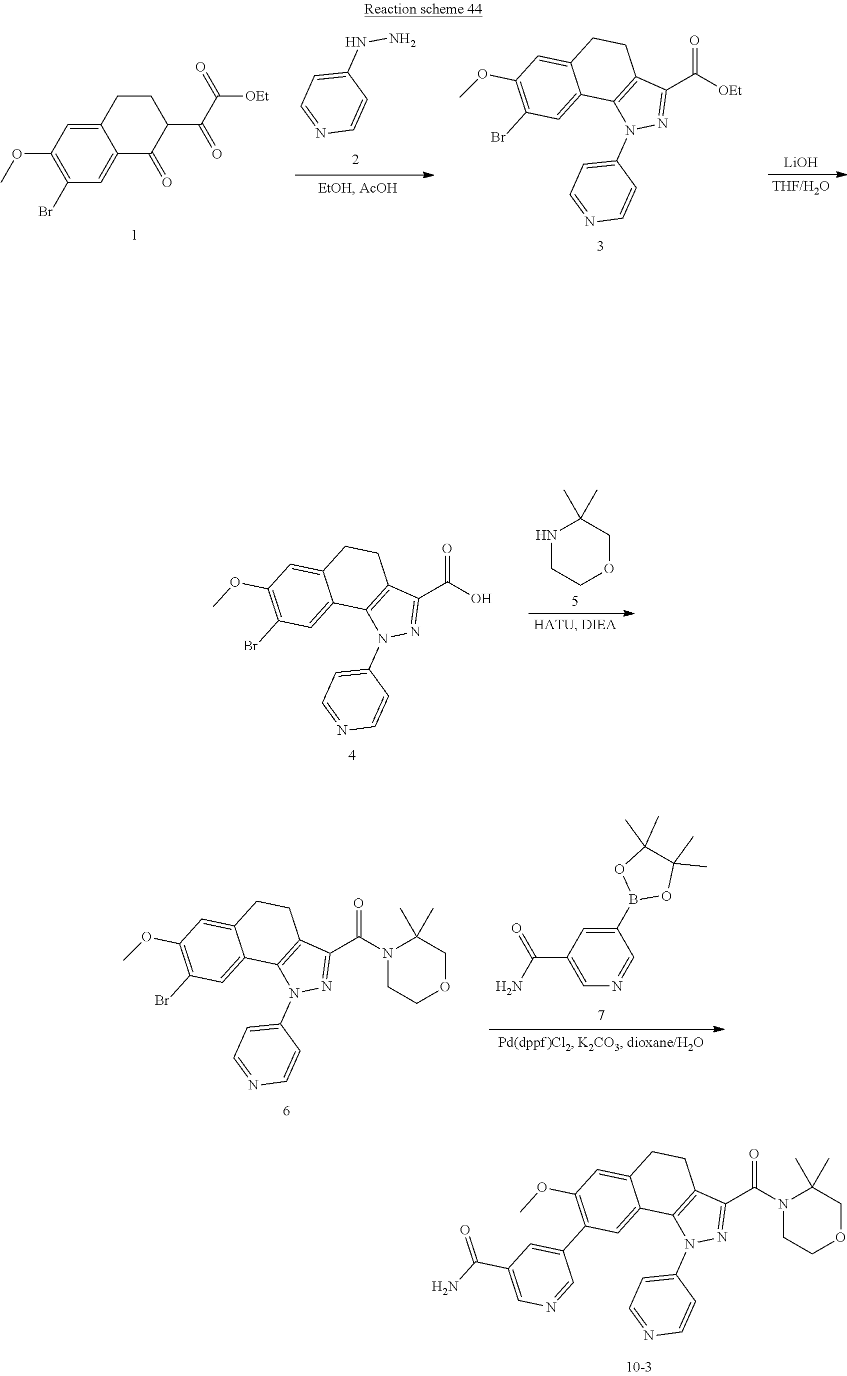

### Synthesis of Compound 10-3

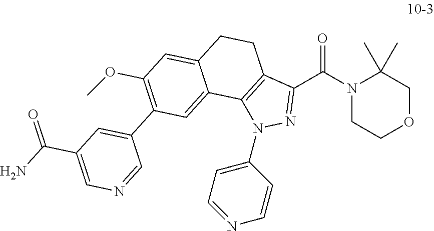 [SMILES: COc1cc2c(cc1-c1cncc(C(N)=O)c1)-c1c(c(C(=O)N3CCOCC3(C)C)nn1-c1ccncc1)CC2]

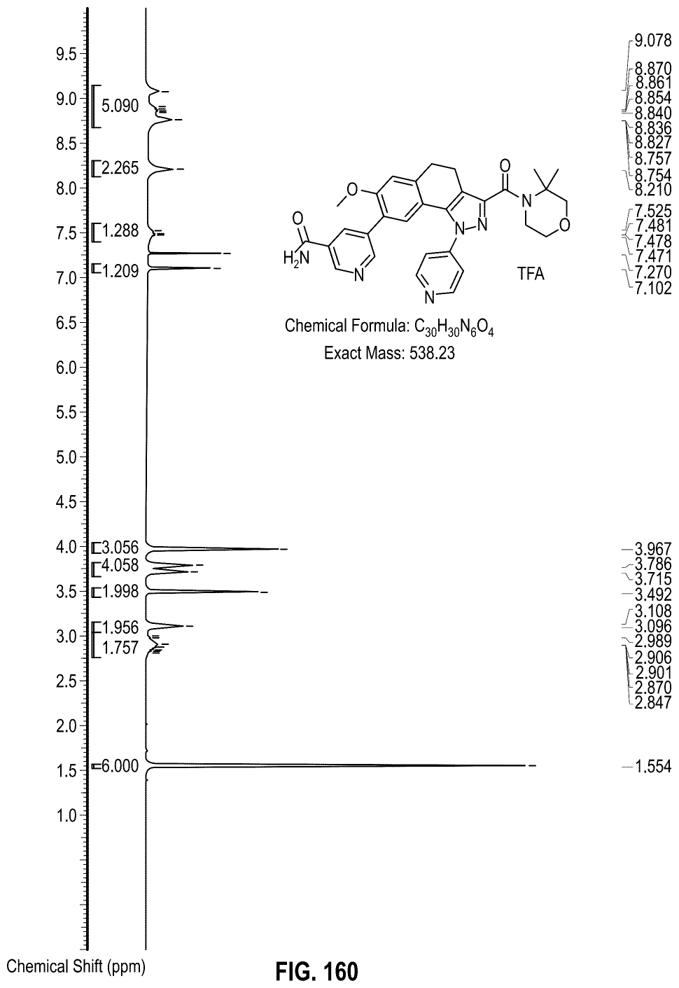

Compound 10-3 was synthesized based on a procedure similar to Reaction scheme 44. LCMS (ESI): m/z [M+H] calcd for C30H31N6O4: 539.23; found: 539.2. 1H NMR (400 MHz, CHLOROFORM-d) δ=9.14-8.68 (m, 5H), 8.21 (br s, 2H), 7.60-7.41 (m, 1H), 7.10 (s, 1H), 3.97 (s, 3H), 3.84-3.67 (m, 4H), 3.49 (s, 2H), 3.10 (br d, J=4.8 Hz, 2H), 3.04-2.76 (m, 2H), 1.55 (s, 6H). FIG. 160 shows the nuclear magnetic resonance of Compound 10-3.

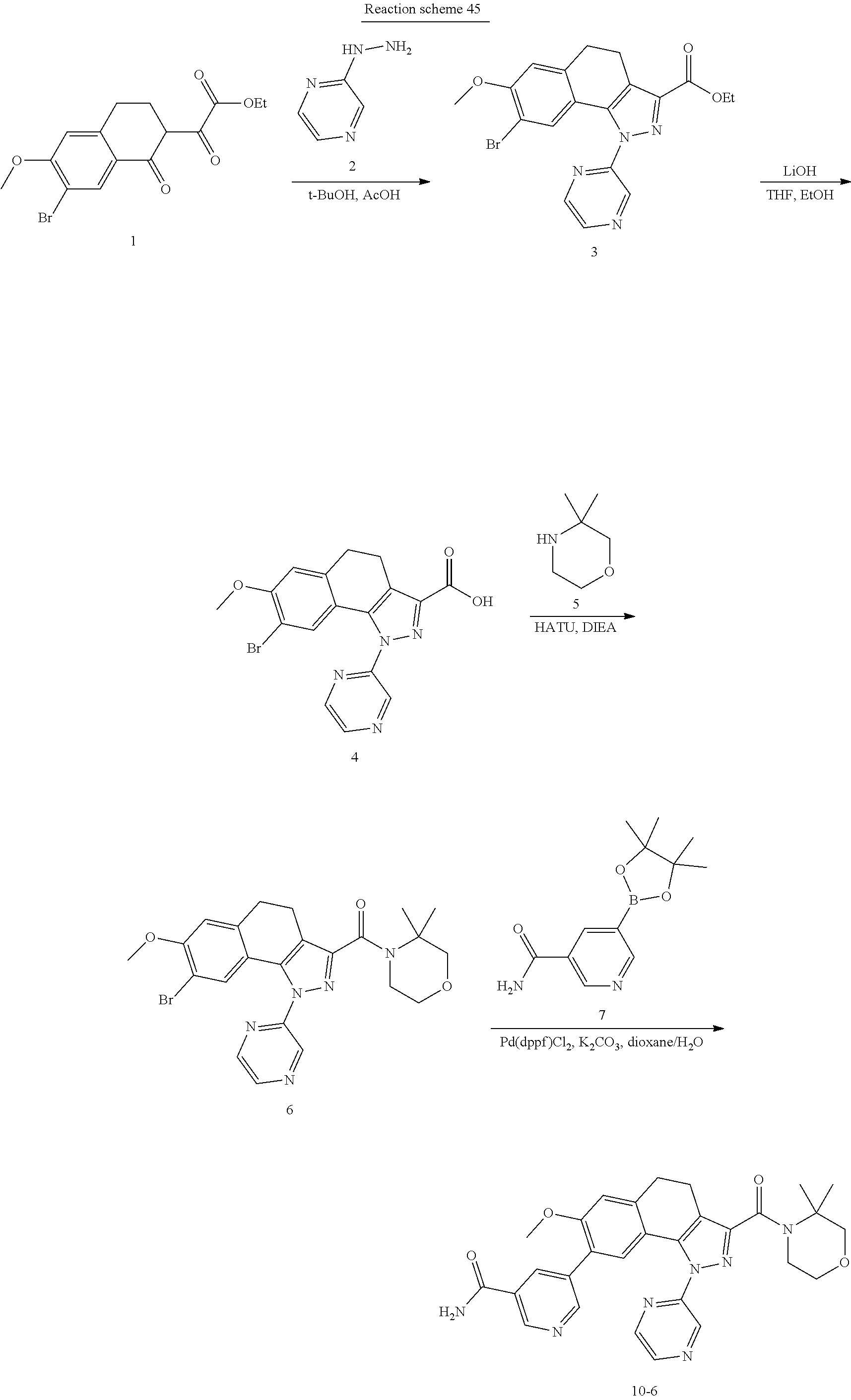

### Synthesis of Compound 10-6

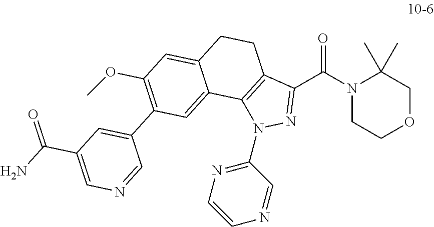 [SMILES: COc1cc2c(cc1-c1cncc(C(N)=O)c1)-c1c(c(C(=O)N3CCOCC3(C)C)nn1-c1cnccn1)CC2]

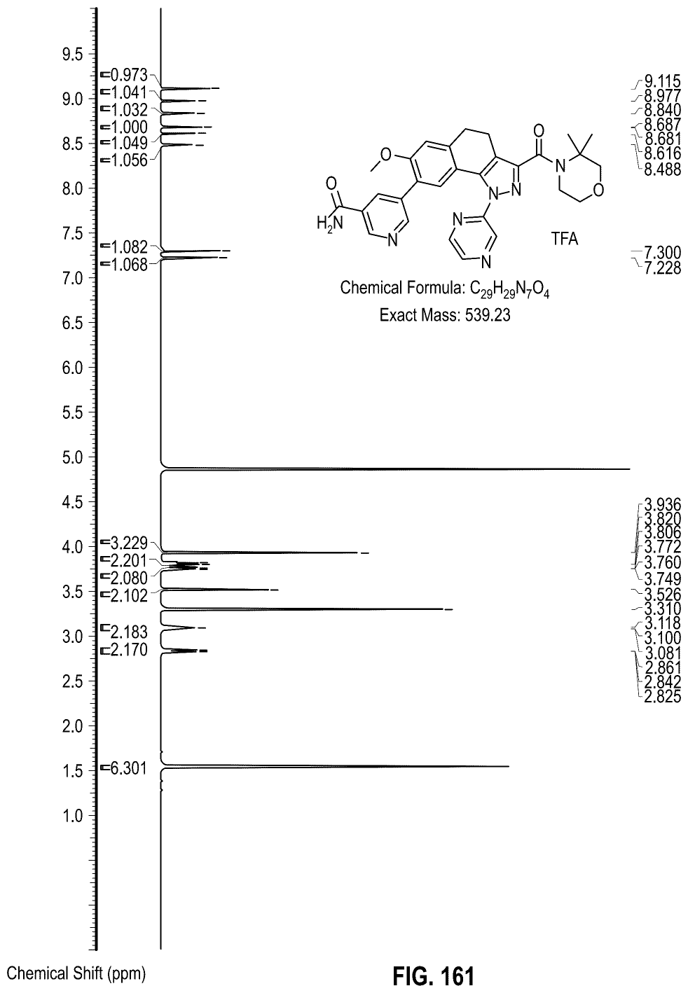

Compound 10-6 was synthesized based on a procedure similar to Reaction scheme 45. LCMS (ESI): m/z [M+H] calcd for C29H30N7O4: 540.23; found: 540.2. 1H NMR (400 MHz, METHANOL-d4) δ=9.11 (s, 1H), 8.98 (s, 1H), 8.84 (s, 1H), 8.68 (d, J=2.6 Hz, 1H), 8.62 (d, J=1.3 Hz, 1H), 8.49 (s, 1H), 7.30 (s, 1H), 7.23 (s, 1H), 3.94 (s, 3H), 3.84-3.79 (m, 2H), 3.78-3.74 (m, 2H), 3.53 (s, 2H), 3.13-3.07 (m, 2H), 2.88-2.81 (m, 2H), 1.57-1.54 (m, 6H). FIG. 161 shows the nuclear magnetic resonance of Compound 10-6.

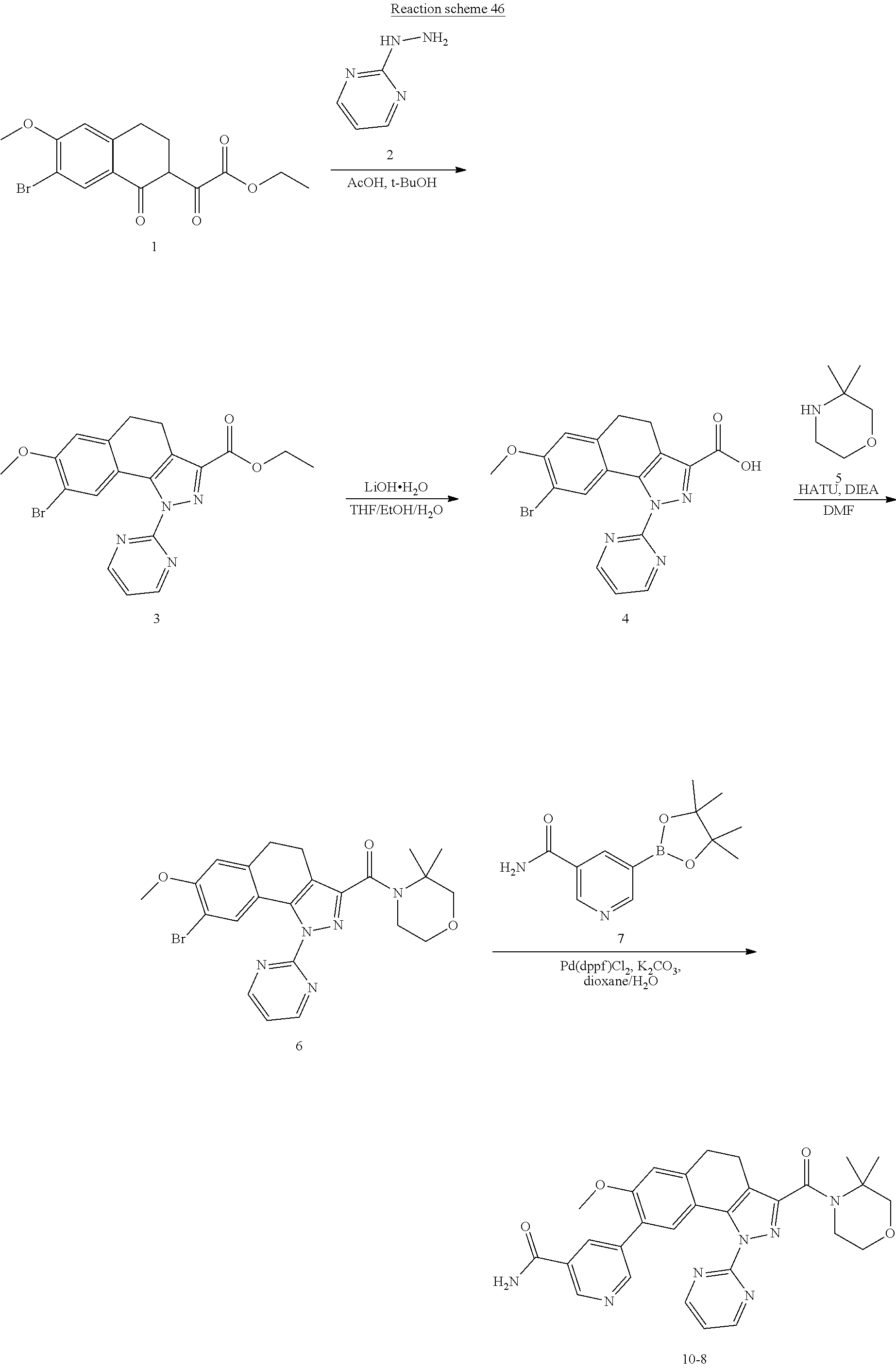

### Synthesis of Compound 10-7

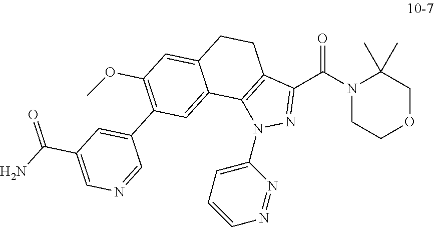 [SMILES: COc1cc2c(cc1-c1cncc(C(N)=O)c1)-c1c(c(C(=O)N3CCOCC3(C)C)nn1-c1cccnn1)CC2]

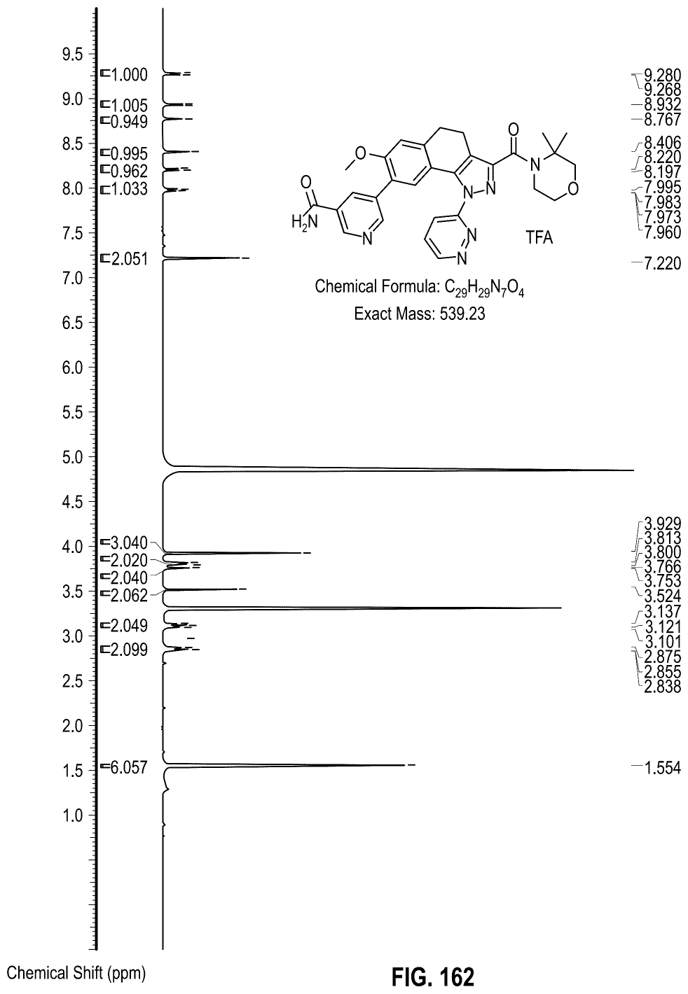

Compound 10-7 was synthesized based on a procedure similar to Reaction scheme 46. LCMS (ESI): m/z [M+H] calcd for C29H30N7O4: 540.23; found: 540.3. 1H NMR (400 MHz, METHANOL-d4) δ=9.27 (d, J=4.8 Hz, 1H), 8.93 (s, 1H), 8.77 (s, 1H), 8.41 (s, 1H), 8.21 (d, J=9.0 Hz, 1H), 7.98 (dd, J=4.8, 8.9 Hz, 1H), 7.22 (s, 2H), 3.93 (s, 3H), 3.81 (br d, J=5.3 Hz, 2H), 3.76 (br d, J=5.3 Hz, 2H), 3.52 (s, 2H), 3.12 (br t, J=7.2 Hz, 2H), 2.89-2.82 (m, 2H), 1.55 (s, 6H). FIG. 162 shows the nuclear magnetic resonance of Compound 10-7.

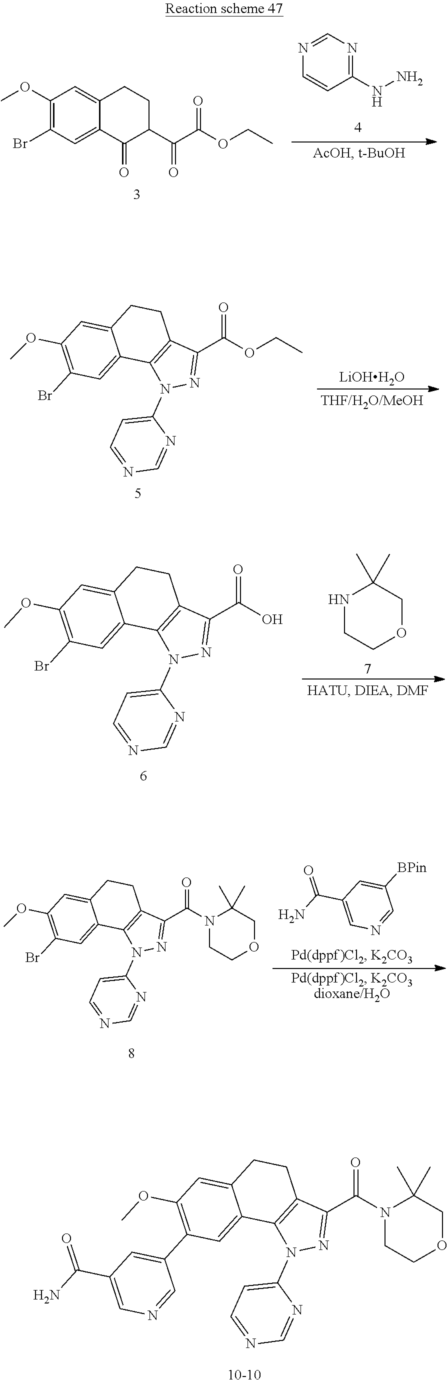

### Synthesis of Compound 10-8

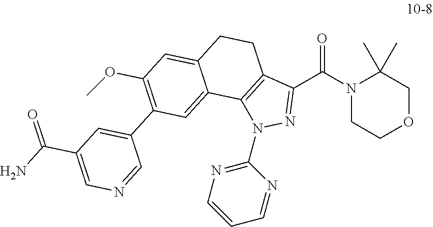 [SMILES: COc1cc2c(cc1-c1cncc(C(N)=O)c1)-c1c(c(C(=O)N3CCOCC3(C)C)nn1-c1ncccn1)CC2]

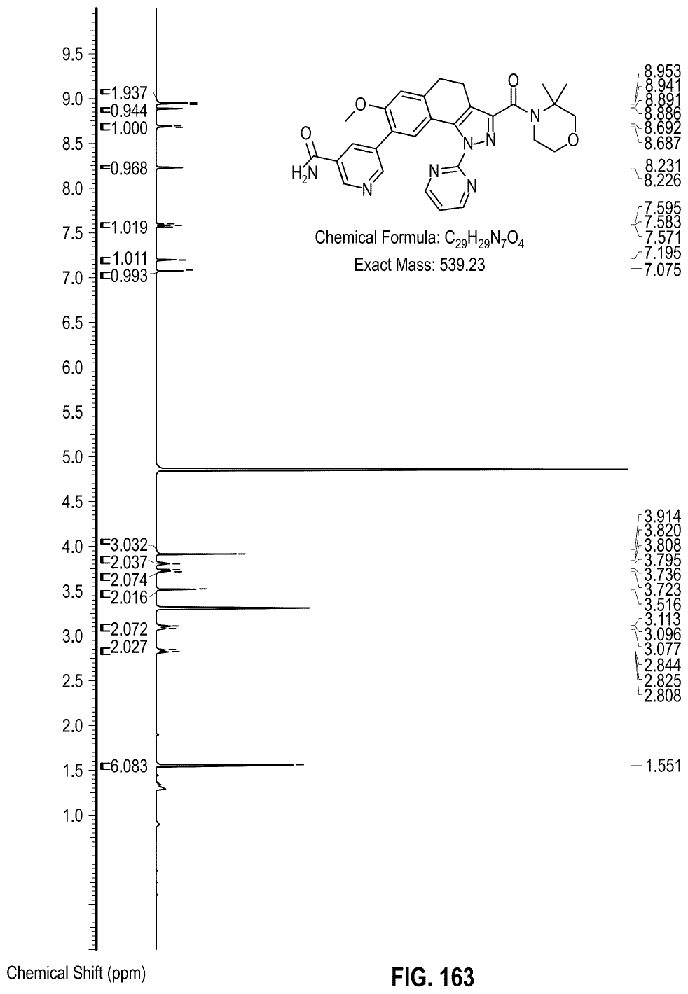

Compound 10-8 was synthesized based on a procedure similar to Reaction scheme 47. LCMS (ESI): m/z [M+H] calcd for C29H30N7O4 540.23; found: 540.2. 1H NMR (400 MHz, METHANOL-d4) δ=8.95 (d, J=4.9 Hz, 2H), 8.89 (d, J=2.1 Hz, 1H), 8.69 (d, J=2.1 Hz, 1H), 8.25-8.21 (m, 1H), 7.58 (t, J=4.8 Hz, 1H), 7.19 (s, 1H), 7.08 (s, 1H), 3.91 (s, 3H), 3.84-3.77 (m, 2H), 3.73 (br d, J=5.1 Hz, 2H), 3.52 (s, 2H), 3.13-3.05 (m, 2H), 2.86-2.79 (m, 2H), 1.55 (s, 6H). FIG. 163 shows the nuclear magnetic resonance of Compound 10-8.

### Synthesis of Compound 10-9

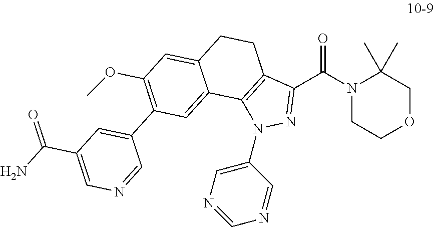 [SMILES: COc1cc2c(cc1-c1cncc(C(N)=O)c1)-c1c(c(C(=O)N3CCOCC3(C)C)nn1-c1cncnc1)CC2]

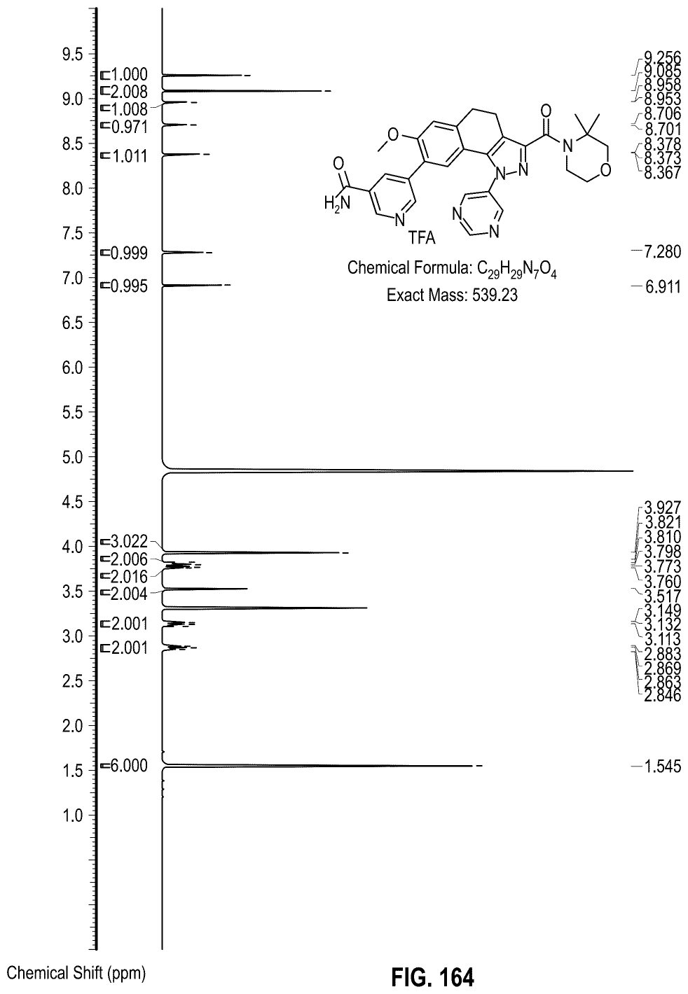

Compound 10-8 was synthesized based on a procedure similar to Reaction scheme 47. LCMS (ESI): m/z [M+H] calcd for C₂₉H₃₀N₇O₄: 540.23; found: 540.4. ¹H NMR (400 MHz, METHANOL-d4) δ=9.26 (s, 1H), 9.08 (s, 2H), 8.96 (d, J=2.0 Hz, 1H), 8.70 (d, J=1.9 Hz, 1H), 8.37 (t, J=2.1 Hz, 1H), 7.28 (s, 1H), 6.91 (s, 1H), 3.93 (s, 3H), 3.83-3.79 (m, 2H), 3.78-3.73 (m, 2H), 3.52 (s, 2H), 3.13 (t, J=7.3 Hz, 2H), 2.90-2.83 (m, 2H), 1.55 (s, 6H). FIG. 164 shows the nuclear magnetic resonance of Compound 10-9.

### Synthesis of Compound 10-10

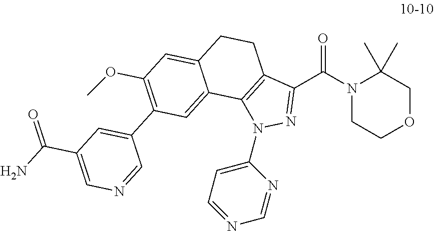 [SMILES: COc1cc2c(cc1-c1cncc(C(N)=O)c1)-c1c(c(C(=O)N3CCOCC3(C)C)nn1-c1ccncn1)CC2]

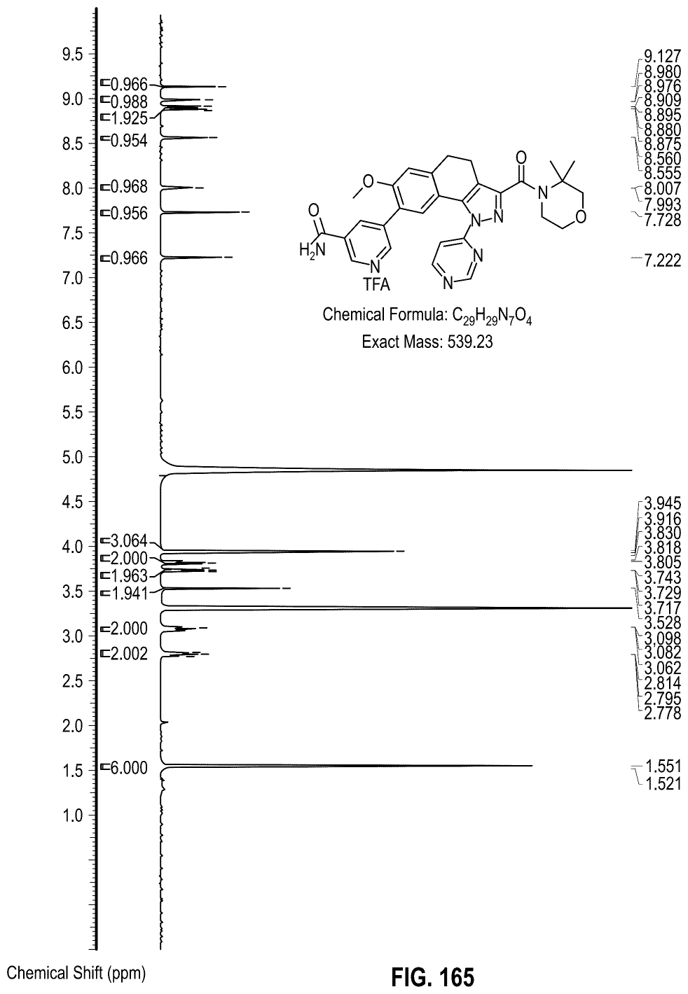

Compound 10-10 was synthesized based on a procedure similar to Reaction scheme 49. LCMS (ESI): m/z [M+H] calcd for C29H29N7O4: 539.23; found: 540.2. 1H NMR: (400 MHz, METHANOL-d4) δ=9.13 (s, 1H), 8.98 (d, J=2.0 Hz, 1H), 8.93-8.85 (m, 2H), 8.56 (t, J=1.9 Hz, 1H), 8.00 (d, J=5.7 Hz, 1H), 7.73 (s, 1H), 7.22 (s, 1H), 3.94 (s, 3H), 3.84-3.79 (m, 2H), 3.77-3.70 (m, 2H), 3.53 (s, 2H), 3.08 (br t, J=7.2 Hz, 2H), 2.80 (t, J=7.3 Hz, 2H), 1.55 (s, 6H). FIG. 165 shows the nuclear magnetic resonance of Compound 10-10.

### Example 11

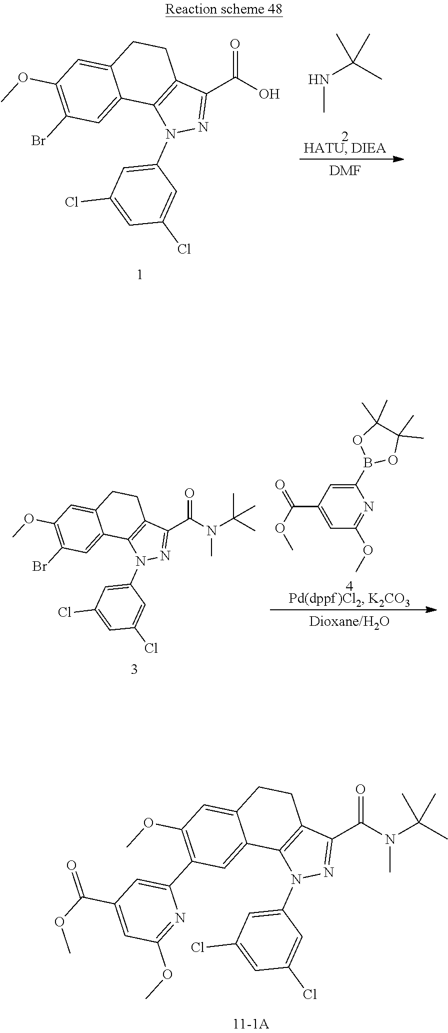

### Synthesis of 8-bromo-N-tert-butyl-1-(3,5-dichlorophenyl)-7-methoxy-N-methyl-4,5-dihydrobenzo[g]indazole-3-carboxamide

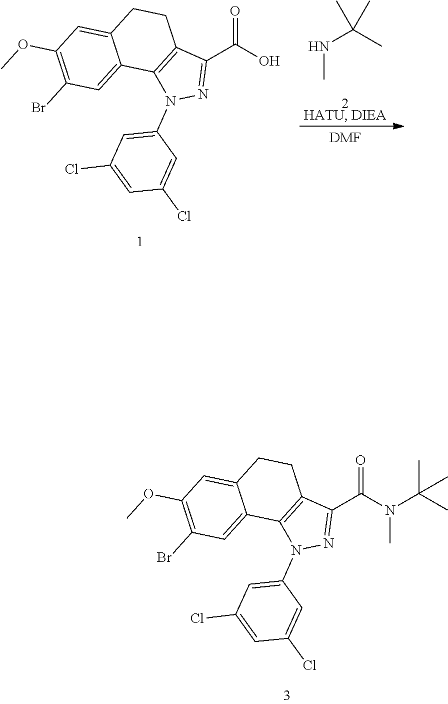

To a solution of 8-bromo-1-(3,5-dichlorophenyl)-7-methoxy-4,5-dihydrobenzo[g]indazole-3-carboxylic acid (80 mg, 170.89 μmol, 1 eq) in DMF (2 mL) was added HATU (97.47 mg, 256.34 μmol, 1.5 eq) and DIEA (66.26 mg, 512.68 μmol, 89.30 μL, 3 eq) in one portion. The mixture was stirred at 25° C. for 0.5 hour. Then to the mixture was added a solution of N,2-dimethylpropan-2-amine (26.81 mg, 307.61 μmol, 36.88 μL, 1.8 eq) in DMF(0.5 mL) in one portion. The mixture was stirred at 25° C. for 2 hours. The reaction mixture was diluted with water (10 mL) and extracted with ethyl acetate (15 mL*2). The combined organic layers were washed with brine (20 mL), dried over anhydrous Na2SO4, filtered and concentrated under reduced pressure to afford 8-bromo-N-tert-butyl-1-(3,5-dichlorophenyl)-7-methoxy-N-methyl-4,5-dihydrobenzo[g]indazole-3-carboxamide (91 mg, 169.37 μmol, 99.11% yield) as a brown solid.

### Synthesis of Compound 11-1A

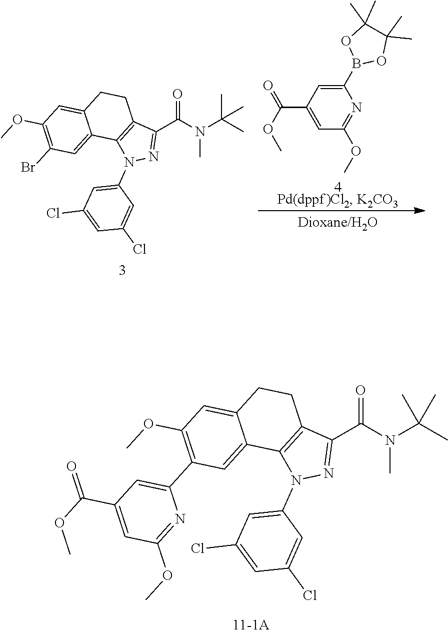

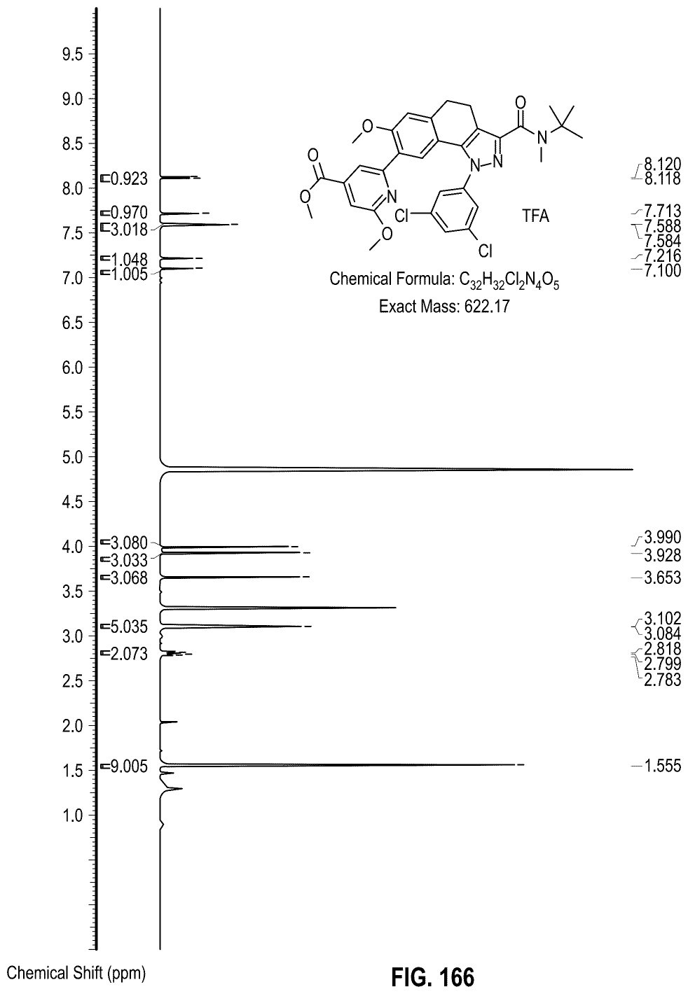

To a solution of 8-bromo-N-tert-butyl-1-(3,5-dichlorophenyl-7-methoxy-N-methyl-4,5-dihydrobenzo[g]indazole-3-carboxamide (91 mg, 169.37 μmol, 1 eq) and methyl 2-methoxy-6-(4,4,5,5-tetramethyl-1,3,2-dioxaborolan-2-yl)pyridine-4-carboxylate (74.47 mg, 254.06 μmol, 1.5 eq) in H2O (1 mL) and dioxane (2 mL) was added Pd(dppf)Cl2 (12.39 mg, 16.94 μmol, 0.1 eq) and K2CO3 (46.82 mg, 338.75 μmol, 2 eq) in one portion. The mixture was stirred at 60° C. for 36.5 hours under N2 atomsphere. The reaction was filtered and the filtrate was concentrated under reduced pressure to give a residue. The residue was purified by prep-HPLC (column: Phenomenex Luna C18 150*25 mm*10 um;mobile phase: [water(TFA)-ACN];gradient:75%-100% B over 9 min) followed by lyophilization. To afford methyl2-[3-[tert-butyl(methyl)carbamoyl]-1-(3,5-dichlorophenyl)-7-methoxy-4,5-dihydrobenzo[g]indazol-8-yl]-6-methoxy-pyridine-4-carboxylate (12.58 mg, 16.37 μmol, 9.67% yield, 96% purity, TFA) as a white solid. LCMS (ESI): m/z [M+H] calcd for C32H33Cl2N4O5: 623.17; found: 623.2. 1H NMR (400 MHz, METHANOL-d4) δ=8.12 (d, J=1.0 Hz, 1H), 7.71 (s, 1H), 7.62-7.55 (m, 3H), 7.22 (s, 1H), 7.10 (s, 1H), 3.99 (s, 3H), 3.93 (s, 3H), 3.65 (s, 3H), 3.13-3.07 (m, 5H), 2.80 (t, J=7.2 Hz, 2H), 1.55 (s, 9H). FIG. 166 shows the nuclear magnetic resonance of Compound 11-1A.

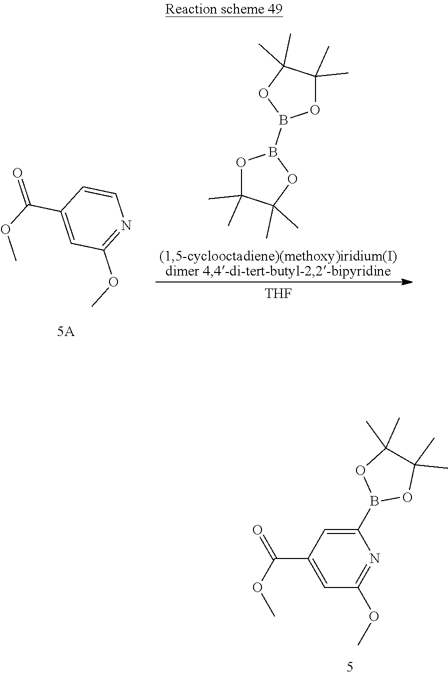

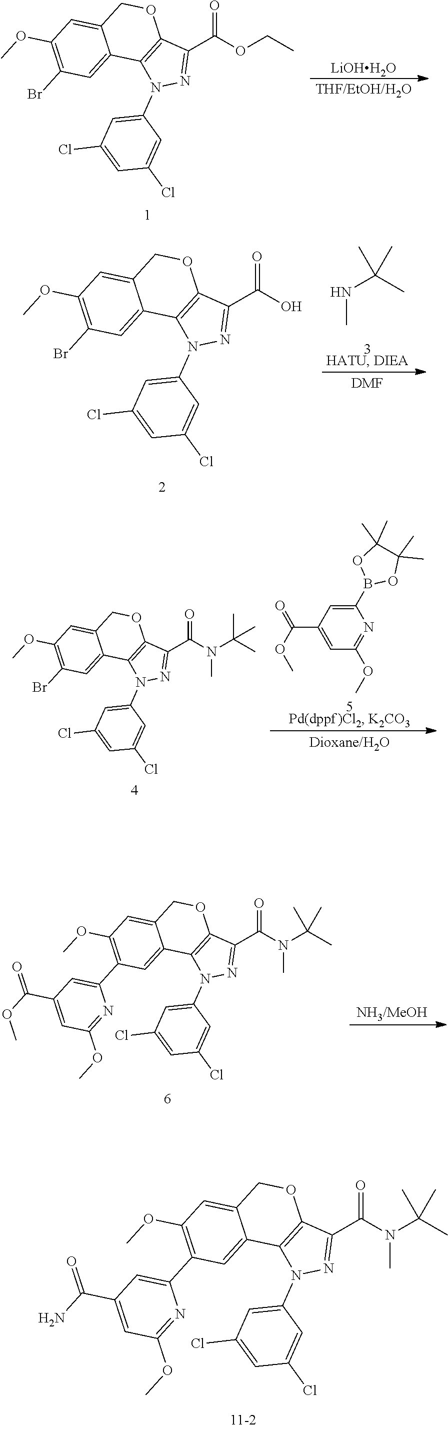

### Synthesis of methyl 2-methoxy-6-(4,4,5,5-tetramethyl-1,3,2-dioxaborolan-2-yl)pyridine-4-carboxylate

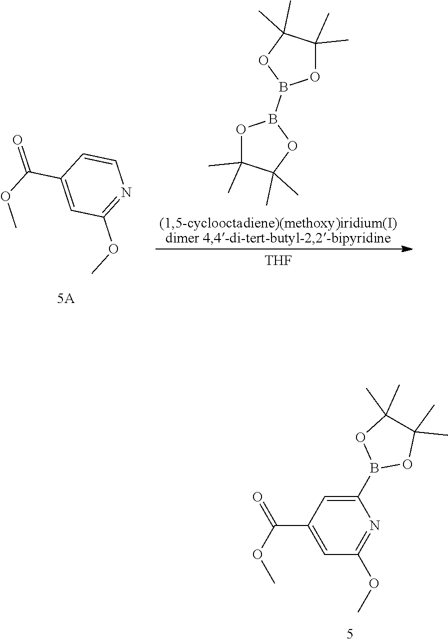

In a glove box, a solution of (1Z,5Z)-cycloocta-1,5-diene;2,4-dimethyl-BLAHbicyclo[1.1.0]butane (79.31 mg, 119.64 μmol, 0.02 eq) and 4,4,5,5-tetramethyl-2-(4,4, 5,5-tetramethyl-1,3,2-dioxaborolan-2-yl)-1,3,2-dioxaborolane (1.52 g, 5.98 mmol, 1 eq) and 4-tert-butyl-2-(4-tert-butyl-2-pyridyl) pyridine (64.22 mg, 239.29 μmol, 0.04 eq) in THF (15 mL) was stirred at 25° C. for 1 min. Then the methyl 2-methoxypyridine-4-carboxylate (1 g, 5.98 mmol, 1 eq) added to the mixture. The reaction mixture was stirred at 25° C. for 16 hr. The reaction mixture was concentrated under reduced pressure to give a residue. The residue was purified by flash silica gel chromatography (ISCO®; 12 g SepaFlash® Silica Flash Column, Eluent of 0˜50% Ethyl acetate/Petroleum ether gradient @50 mL/min). Compound methyl 2-methoxy-6-(4,4,5,5-tetramethyl-1,3,2-dioxaborolan-2-yl) pyridine-4-carboxylate (1.09 g, 3.72 mmol, 72.67% yield) was obtained as a white solid. 1H NMR (400 MHz, CHLOROFORM-d) δ=7.95 (d, J=1.2 Hz, 1H), 7.34 (d, J=1.2 Hz, 1H), 4.05 (s, 3H), 3.93 (s, 3H), 1.39 (s, 12H).

### Synthesis of Compound 11-2

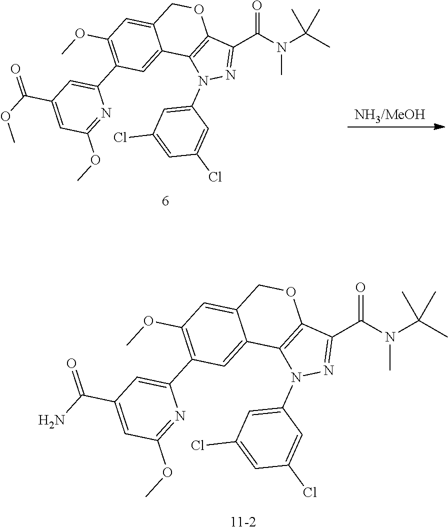 [SMILES: COC(=O)c1cc(OC)nc(-c2cc3c(cc2OC)COc2c(C(=O)N(C)C(C)(C)C)nn(-c4cc(Cl)cc(Cl)c4)c2-3)c1 ; "NH3/MeOH" ; COc1cc(C(N)=O)cc(-c2cc3c(cc2OC)COc2c(C(=O)N(C)C(C)(C)C)nn(-c4cc(Cl)cc(Cl)c4)c2-3)n1]

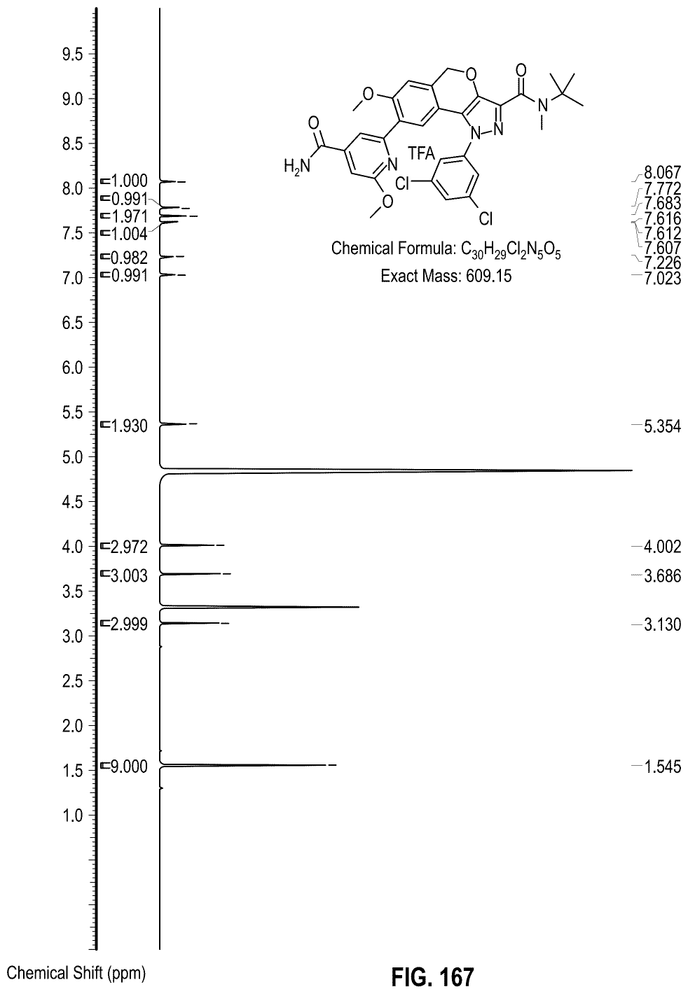

A mixture of methyl 2-[3-[tert-butyl(methyl)carbamoyl]-1-(3,5-dichlorophenyl)-7-methoxy-5H-isochromeno[4,3-c]pyrazol-8-yl]-6-methoxy-pyridine-4-carboxylate (70 mg, 111.91 μmol, 1 eq) in NH3/MeOH (6 mL) was stirred at 60° C. for 16 h. The reaction mixture was concentrated under reduced pressure to give a residue. The residue was purified by prep-HPLC (column: Phenomenex Luna C18 150*25 mm*10 um; mobile phase: [water (TFA) -ACN]; gradient: 56%-86% B over 9 min), followed by Lyophilization. Compound N-tert-butyl-8-(4-carbamoyl-6-methoxy-2-pyridyl)-1-(3,5-dichlorophenyl)-7-methoxy-N-methyl-5H-isochromeno[4,3-c]pyrazole-3-carboxamide (50.88 mg, 70.23 μmol, 62.75% yield, 100% purity, TFA) was obtained as a yellow solid. LCMS (ESI): m/z [M+H] calcd for C30H30Cl2N5O5: 610.15; found: 610.3. 1H NMR (400 MHz, METHANOL-d4) δ=8.07 (d, J=1.2 Hz, 1H), 7.77 (s, 1H), 7.69 (d, J=2.0 Hz, 2H), 7.61 (t, J=2.0 Hz, 1H), 7.23 (s, 1H), 7.02 (d, J=1.2 Hz, 1H), 5.35 (s, 2H), 4.00 (s, 3H), 3.69 (s, 3H), 3.13 (s, 3H), 1.54 (s, 9H). FIG. 167 shows the nuclear magnetic resonance of Compound 11-2.

### Synthesis of Compound 11-1

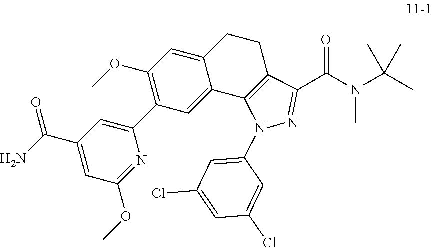 [SMILES: COc1cc(C(N)=O)cc(-c2cc3c(cc2OC)CCc2c(C(=O)N(C)C(C)(C)C)nn(-c4cc(Cl)cc(Cl)c4)c2-3)n1]

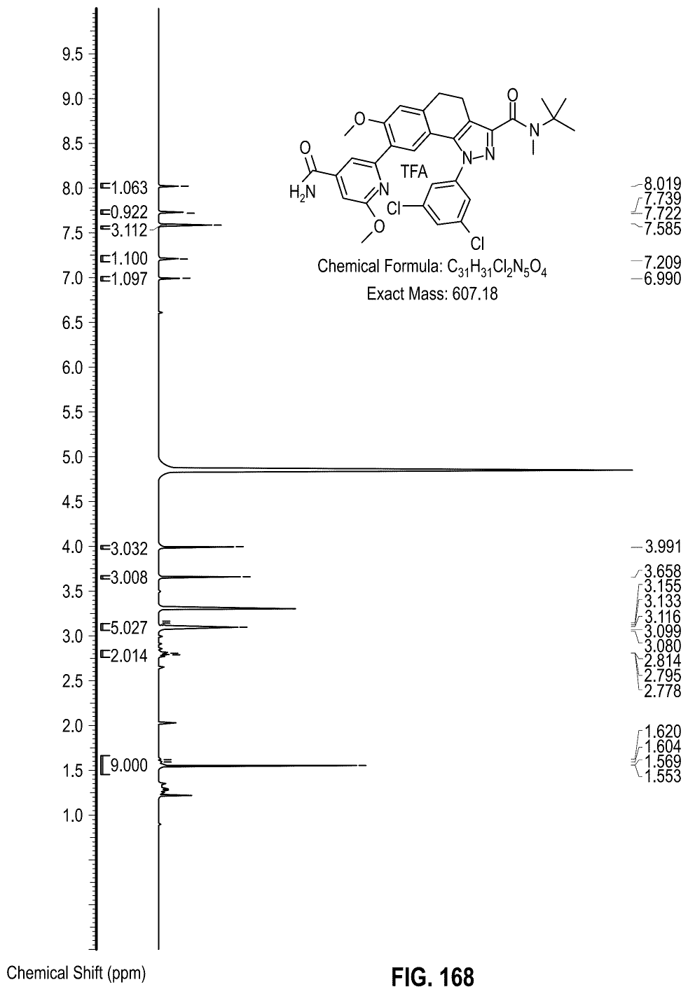

Compound 11-1 was synthesized based on a procedure similar to Reaction scheme 49. LCMS (ESI): m/z [M+H] calcd for C31H32Cl2N5O4: 608.18; found: 608.3. 1H NMR (400 MHz, METHANOL-d4) δ=8.02 (d, J=1.0 Hz, 1H), 7.72 (s, 1H), 7.58 (s, 3H), 7.21 (s, 1H), 6.99 (d, J=1.0 Hz, 1H), 3.99 (s, 3H), 3.66 (s, 3H), 3.13-3.06 (m, 5H), 2.83-2.76 (m, 2H), 1.55 (s, 9H). FIG. 168 shows the nuclear magnetic resonance of Compound 11-1.

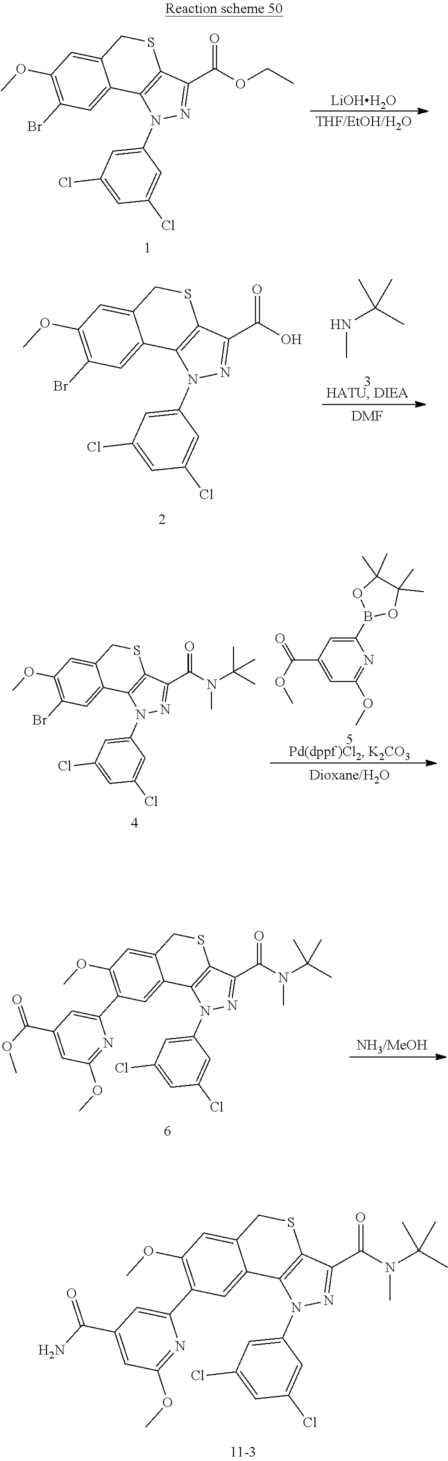

### Synthesis of methyl 2-[3-[tert-butyl(methyl)carbamoyl]-1-(3,5-dichlorophenyl)-7-methoxy-5H-isothiochromeno[4,3-c]pyrazol-8-yl]-6-methoxy-pyridine-4-carboxylate

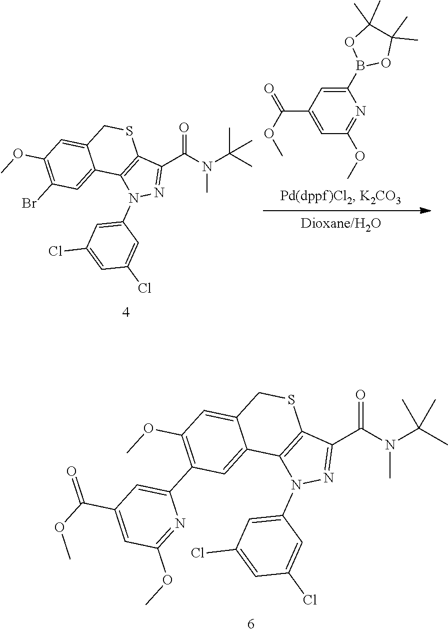

8-bromo-N-tert-butyl-1-(3,5-dichlorophenyl)-7-methoxy-N-methyl-5H-isothiochromeno[4,3-c]pyrazole-3-carboxamide (80 mg, 144.06 μmol, 1 eq), methyl 2-methoxy-6-(4,4,5,5-tetramethyl-1,3,2-dioxaborolan-2-yl)pyridine-4-carboxylate (46.45 mg, 158.47 μmol, 1.1 eq), K2CO3 (39.82 mg, 288.13 μmol, 2 eq) and Pd(dppf)Cl2 (21.08 mg, 28.81 μmol, 0.2 eq) in dioxane (2 mL) and H2O (0.2 mL) was degassed and then heated to 60° C. for 16 hours under N2. The residue was diluted with EtOAc (10 mL) and then filtered through a celite pad. The filtrate was concentrated. The residue was purified by flash silica gel chromatography (ISCO®; 4 g SepaFlash® Silica Flash Column, Eluent of 0˜30% Ethyl acetate/Petroleum ethergradient @30 mL/min). Compound methyl 2-[3-[tert-butyl(methyl)carbamoyl]-1-(3,5-dichlorophenyl)-7-methoxy-5H-isothiochromeno[4,3-c]pyrazol-8-yl]-6-methoxy-pyridine-4-carboxylate (56 mg, 87.29 μmol, 60.59% yield) was obtained as a colorless oil. LCMS (ESI): m/z [M+H] calcd for C31H31Cl2N4O5S: 641.13; found: 641.2.

### Synthesis of Compound 11-3

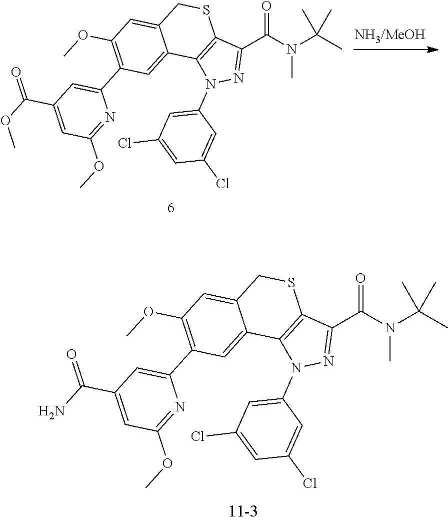 [SMILES: COC(=O)c1cc(OC)nc(-c2cc3c(cc2OC)CSc2c(C(=O)N(C)C(C)(C)C)nn(-c4cc(Cl)cc(Cl)c4)c2-3)c1 ; COc1cc(C(N)=O)cc(-c2cc3c(cc2OC)CSc2c(C(=O)N(C)C(C)(C)C)nn(-c4cc(Cl)cc(Cl)c4)c2-3)n1]

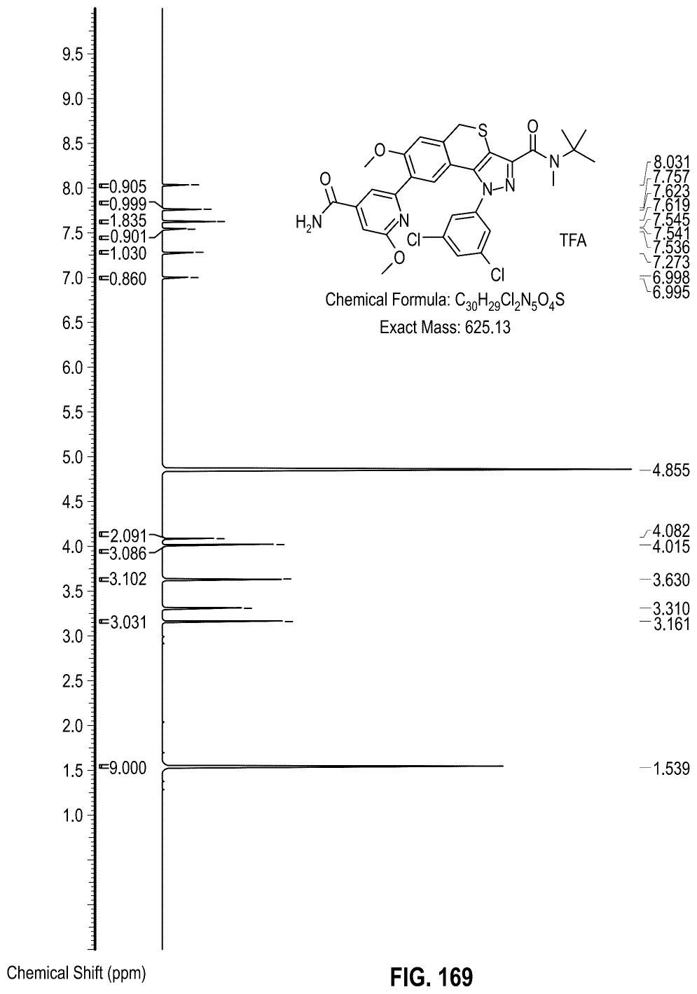

A mixture of methyl 2-[3-[tert-butyl (methyl) carbamoyl]-1-(3,5-dichlorophenyl)-7-methoxy-5H-isothiochromeno[4,3-c]pyrazol-8-yl]-6-methoxy-pyridine-4-carboxylate (50 mg, 77.93 μmol, 1 eq) in NH3/MeOH (7 M, 5 mL, 449.09 eq) was stirred at 60° C. for 16 hr. The reaction mixture was concentrated. The residue was purified by prep-HPLC (column: Phenomenex luna C18 150*25 mm*10 um; mobile phase: [water (TFA) -ACN]; gradient: 65%-95% B over min). Compound N-tert-butyl-8-(4-carbamoyl-6-methoxy-2-pyridyl)-1-(3,5-dichlorophenyl)-7-methoxy-N-methyl-5H-isothiochromeno[4,3-c]pyrazole-3-carboxamide (15 mg, 20.25 μmol, 25.99% yield, TFA) was obtained as a yellow solid. LCMS (ESI): m/z [M+H] calcd for C30H30Cl2N5O4S: 626.13; found: 626.2. 1H NMR (400 MHz, METHANOL-d4) δ=8.03 (d, J=1.1 Hz, 1H), 7.76 (s, 1H), 7.62 (d, J=1.7 Hz, 2H), 7.54 (t, J=1.8 Hz, 1H), 7.27 (s, 1H), 7.00 (d, J=1.2 Hz, 1H), 4.08 (s, 2H), 4.02 (s, 3H), 3.63 (s, 3H), 3.16 (s, 3H), 1.54 (s, 9H). FIG. 169 shows the nuclear magnetic resonance of Compound 11-3.

### Example 12

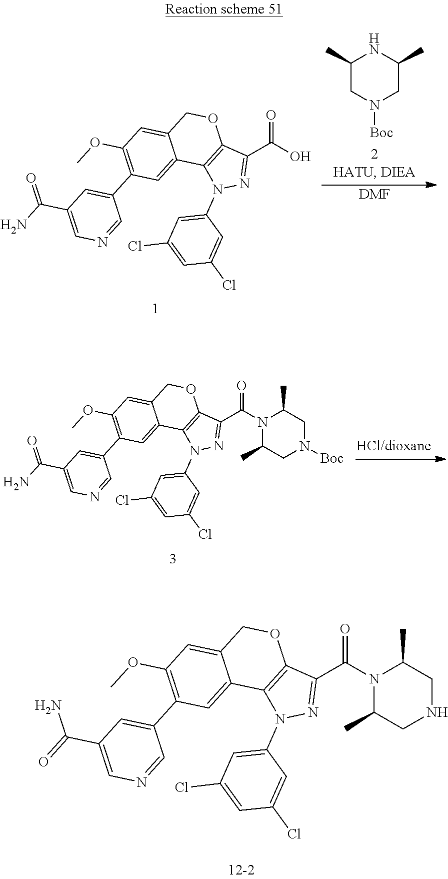

### Synthesis of tert-butyl(3R,5S)-4-[8-(5-carbamoyl-3-pyridyl)-1-(3,5-dichlorophenyl)-7-methoxy-5H-isochromeno[4,3-c]pyrazole-3-carbonyl]-3,5-dimethyl-piperazine-1-carboxylate

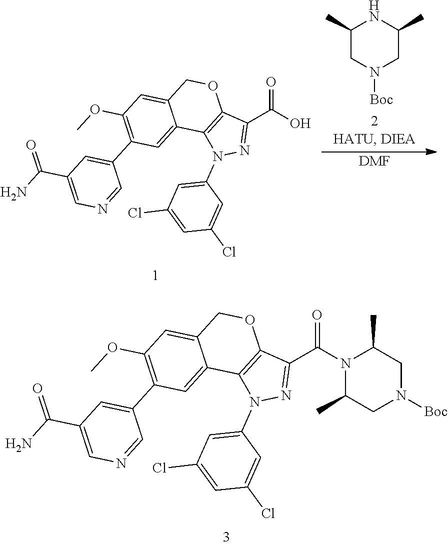

To a solution of 8-(5-carbamoyl-3-pyridyl)-1-(3,5-dichlorophenyl)-7-methoxy-5H-isochromeno[4,3-c]pyrazole-3-carboxylic acid (60 mg, 117.34 μmol, 1 eq) in DMF (2 mL) was added HATU (44.62 mg, 117.34 μmol, 1 eq) and DIEA (15.17 mg, 117.34 μmol, 20.44 μL, 1 eq). The mixture was stirred at 25° C. for 0.5 hour. Then to the mixture was added a mixture of tert-butyl (3R,5S)-3,5-dimethylpiperazine-1-carboxylate (45.27 mg, 211.22 μmol, 1.8 eq) in DMF (0.5 mL). The mixture was stirred at 25° C. for 16 hours. The reaction mixture was diluted with water (5 mL) and filtered. The filter cake was dried in vacuum to afford tert-butyl (3R,5S)-4-[8-(5-carbamoyl-3-pyridyl)-1-(3,5-dichlorophenyl)-7-methoxy-5H-isochromeno[4,3-c]pyrazole-3-carbonyl]-3,5-dimethyl-piperazine-1-carboxylate (54 mg, 76.31 μmol, 65.03% yield) as a brown solid. 1H NMR (400 MHz, METHANOL-d4) δ=8.91 (s, 1H), 8.63 (s, 1H), 8.30 (s, 1H), 7.72 (s, 2H), 7.59 (s, 1H), 7.24 (s, 1H), 6.99 (s, 1H), 5.36 (s, 2H), 4.05-3.99 (m, 1H), 3.91 (s, 3H), 3.16-3.09 (m, 2H), 2.63-2.58 (m, 1H), 1.50 (s, 6H), 1.32-1.26 (m, 2H), 1.08 (br d, J=6.5 Hz, 9H).

### Synthesis of Compound 12-2

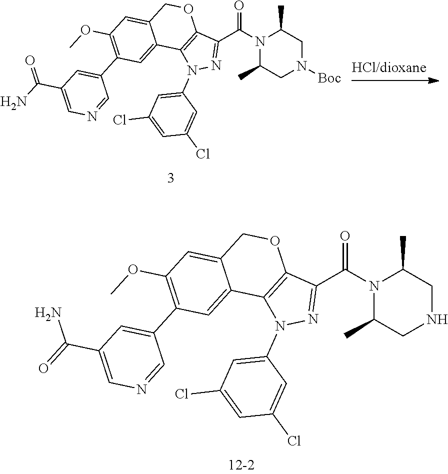 [SMILES: COc1cc2c(cc1-c1cncc(C(N)=O)c1)-c1c(c(C(=O)N3[C@H](C)CN(C(=O)OC(C)(C)C)C[C@@H]3C)nn1-c1cc(Cl)cc(Cl)c1)OC2 ; COc1cc2c(cc1-c1cncc(C(N)=O)c1)-c1c(c(C(=O)N3[C@H](C)CNC[C@@H]3C)nn1-c1cc(Cl)cc(Cl)c1)OC2]

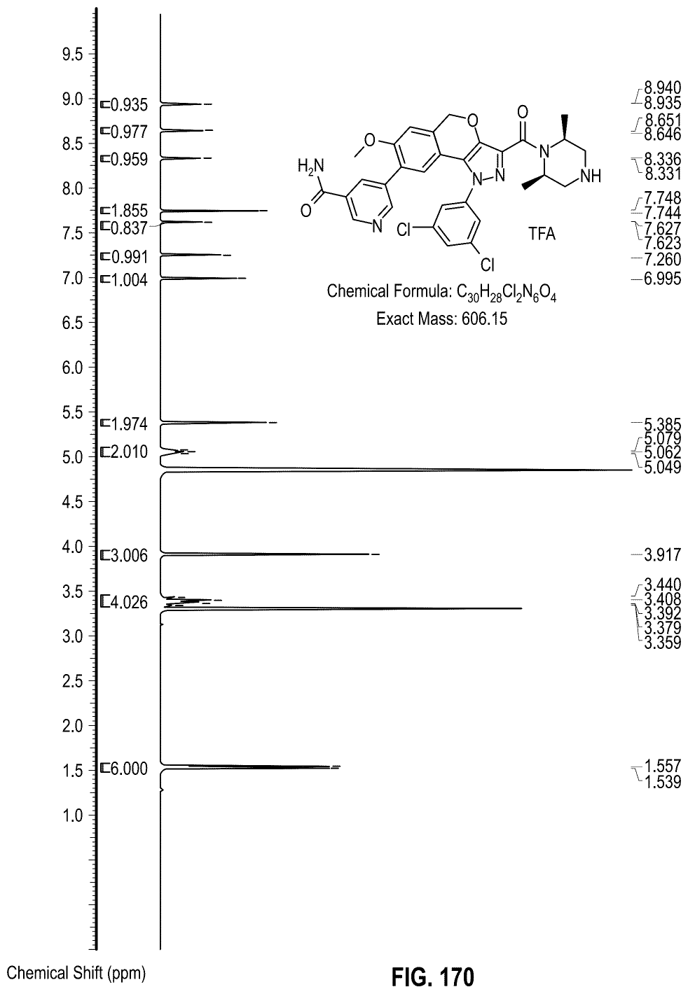

To a solution of tert-butyl(3S,5R)-4-[8-(5-carbamoyl-3-pyridyl)-1-(3,5-dichlorophenyl)-7-methoxy-5H-isochromeno[4,3-c]pyrazole-3-carbonyl]-3,5-dimethyl-piperazine-1-carboxylate (54 mg, 76.31 μmol, 1 eq) in dioxane (1 mL) was added HCl/dioxane (2 M, 381.57 μL, 10 eq) in one portion. The mixture was stirred at 25° C. for 1 hour. The reaction mixture was concentrated under reduced pressure to give a residue. The residue was purified by prep-HPLC (column: Phenomenex Luna C18 150*25 mm*10 um;mobile phase:[water(TFA)-ACN];gradient:18%-48% B over 9 min) followed by lyophilization to afford 5-[1-(3,5-dichlorophenyl)-3-[(2R,6S)-2,6-dimethylpiperazine-1-carbonyl]-7-methoxy-5H-isochromeno[4,3-c]pyrazol-8-yl]pyridine-3-carboxamide (24.5 mg, 33.96 μmol, 44.50% yield, 100% purity, TFA) as a gray solid. LCMS (ESI): m/z[M+H] calcd for C30H29Cl2N6O4: 607.15; found: 607.3. 1H NMR (400 MHz, METHANOL-d4) δ=8.94 (d, J=2.0 Hz, 1H), 8.65 (d, J=2.0 Hz, 1H), 8.37-8.31 (m, 1H), 7.75 (d, J=1.8 Hz, 2H), 7.63 (t, J=1.7 Hz, 1H), 7.26 (s, 1H), 6.99 (s, 1H), 5.38 (s, 2H), 5.11-5.01 (m, 2H), 3.92 (s, 3H), 3.48-3.33 (m, 4H), 1.55 (d, J=7.2 Hz, 6H). FIG. 170 shows the nuclear magnetic resonance of Compound 12-2.

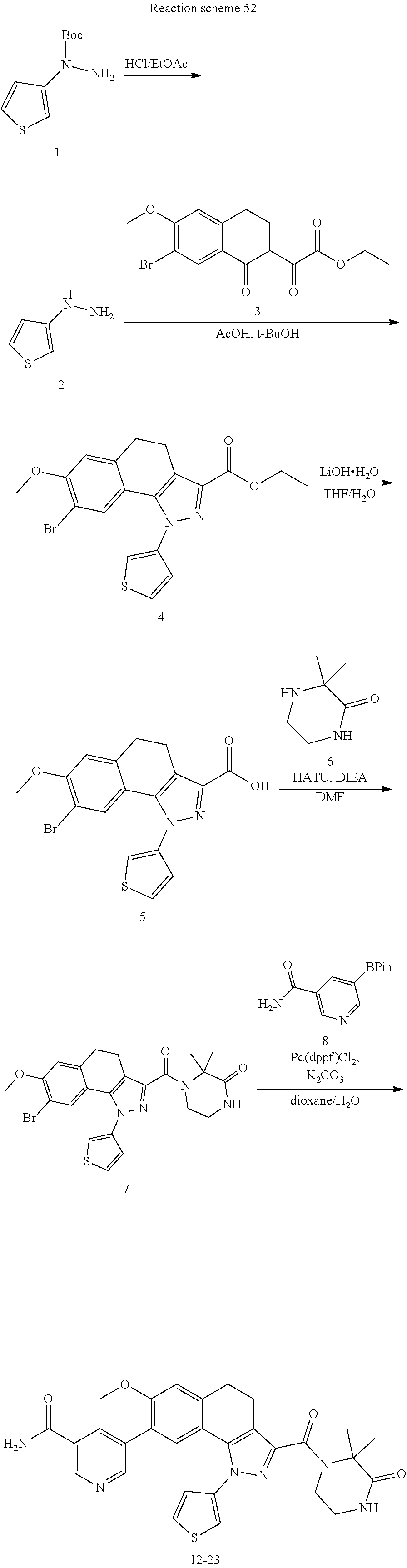

### Synthesis of 3-thienylhydrazine

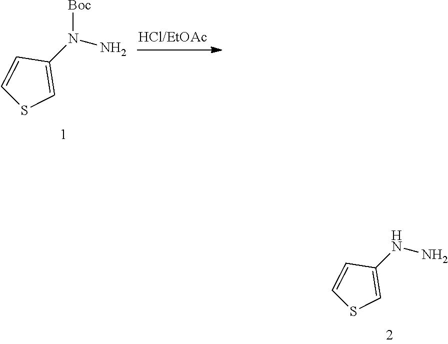 [SMILES: CC(C)(C)OC(=O)N(N)c1ccsc1 ; NNc1ccsc1]

A mixture of tert-butyl N-amino-N-(3-thienyl)carbamate (130 mg, 606.67 μmol, 1 eq) in EtOAc (0.5 mL) was added HCl/EtOAc (2 M, 6.07 mL, 20 eq). The mixture was stirred at 25° C. for 1 h. The reaction mixture was concentrated under reduced pressure to afford compound 3-thienylhydrazine (80 mg, crude, HCl) as a brown solid.

### Synthesis of ethyl 8-bromo-7-methoxy-1-(3-thienyl)-4,5-dihydrobenzo[g]indazole-3-carboxylate

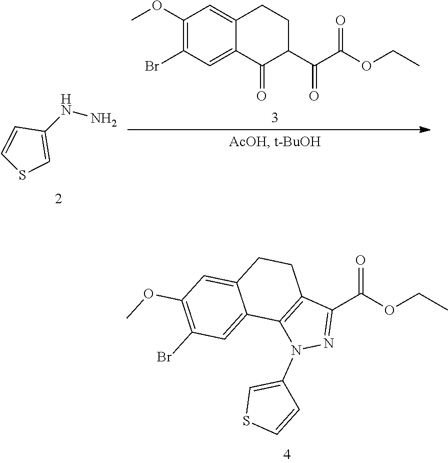

To a mixture of ethyl 2-(7-bromo-6-methoxy-1-oxo-tetralin-2-yl)-2-oxo-acetate (188.64 mg, 531.10 μmol, 1 eq), 3-thienylhydrazine (80 mg, 531.10 μmol, 1 eq, HCl) in t-BuOH (1 mL) was added AcOH (1.12 g, 18.59 mmol, 1.06 mL, 35 eq). The mixture was stirred at 90° C. for 2 h. The reaction mixture was diluted with H₂O. The mixture was filtered, the filter cake was dried under reduced pressure to afford ethyl 8-bromo-7-methoxy-1-(3-thienyl)-4,5-dihydrobenzo[g]indazole-3-carboxylate (229 mg, crude) was obtained as a brown solid.

### Synthesis of 8-bromo-7-methoxy-1-(3-thienyl)-4,5-dihydrobenzo[g]indazole-3-carboxylic acid

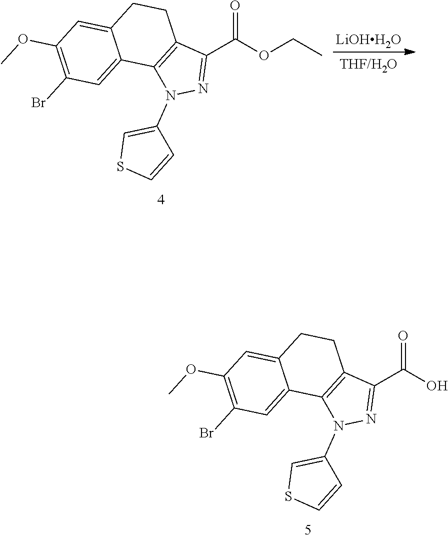

A mixture of ethyl 8-bromo-7-methoxy-1-(3-thienyl)-4,5-dihydrobenzo[g]indazole-3-carboxylate (229 mg, 528.48 μmol, 1 eq) and LiOH. H2O (110.88 mg, 2.64 mmol, 5 eq) in EtOH (3 mL) and H2O (1.5 mL) was stirred at 25° C. for 2 h. The reaction mixture was concentrated under reduced pressure to remove EtOH. The residue was diluted with H2O (4 mL) and the pH was adjusted to 4 with 1 M HCl, and then a lot of solid was precipitated out. The mixture was filtered, the filter cake was dried under reduced pressure to afford compound 8-bromo-7-methoxy-1-(3-thienyl)-4,5-dihydrobenzo[g]indazole-3-carboxylic acid (200 mg, crude) as a brown solid.

### Synthesis of 4-[8-bromo-7-methoxy-1-(3-thienyl)-4,5-dihydrobenzo[g]indazole-3-carbonyl]-3,3-dimethyl-piperazin-2-one

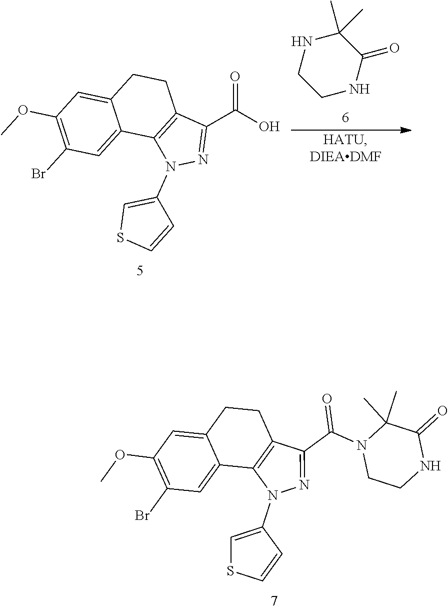

A mixture of 8-bromo-7-methoxy-1-(3-thienyl)-4,5-dihydrobenzo[g]indazole-3-carboxylic acid (60 mg, 148.05 μmol, 1 eq), HATU (84.44 mg, 222.08 μmol, 1.5 eq) and DIEA (76.54 mg, 592.20 μmol, 103.15 μL, 4 eq) in DMF (1 mL) was stirred at 25° C. for 15 min. To the mixture was added 3, 3-dimethylpiperazin-2-one (22.77 mg, 177.66 μmol, 1.2 eq). The mixture was stirred at 25° C. for 1 h. The reaction mixture was diluted with H₂O (10 mL) and extracted with EtOAc (10 mL*3). The combined organic layers were washed with brine (10 mL*2), dried over anhydrous Na₂SO₄, filtered and concentrated under reduced pressure to give a residue. The residue was purified by column chromatography (SiO₂, Petroleum ether/Ethyl acetate=1/0 to ½) to afford 4-[8-bromo-7-methoxy-1-(3-thienyl)-4,5-dihydrobenzo[g]indazole-3-carbonyl]-3,3-dimethyl-piperazin-2-one (53 mg, 102.83 μmol, 69.45% yield) as a white solid.

### Synthesis of Compound 12-23

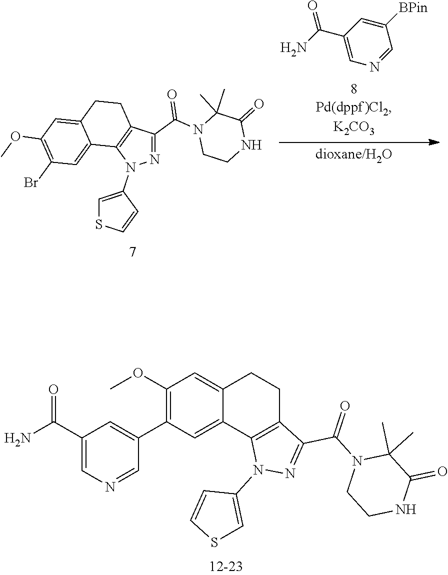

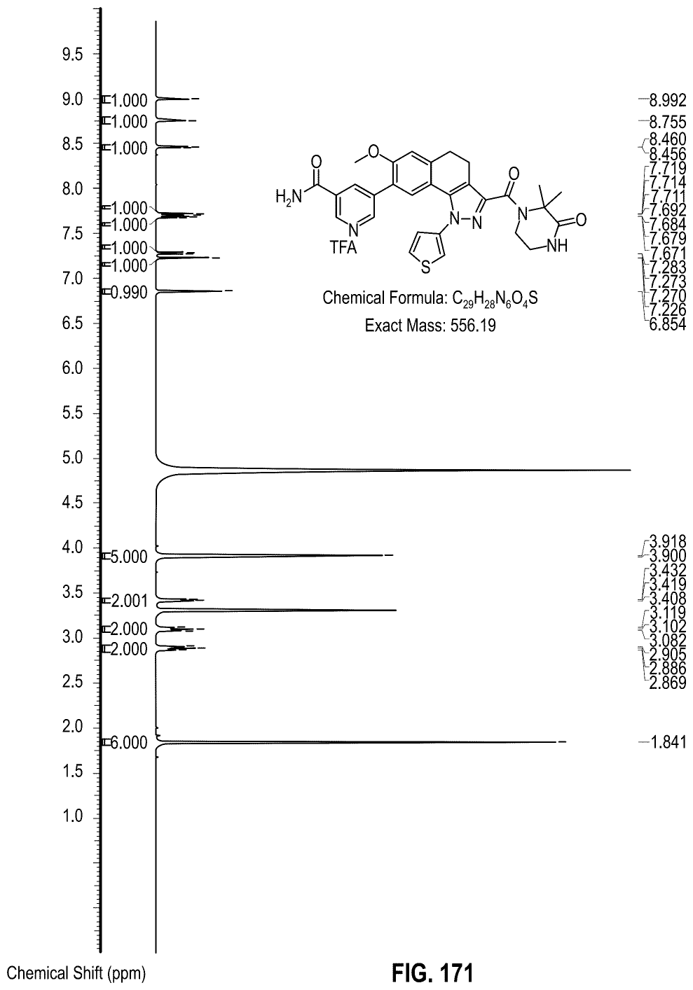

To a mixture of 4-[8-bromo-7-methoxy-1-(3-thienyl)-4,5-dihydrobenzo[g]indazole-3-carbonyl]-3,3-dimethyl-piperazin-2-one (50 mg, 97.01 μmol, 1 eq), 5-(4,4,5,5-tetramethyl-1,3,2-dioxaborolan-2-yl)pyridine-3-carboxamide (36.10 mg, 145.51 mol, 1.5 eq) and K2CO3 (40.22 mg, 291.02 μmol, 3 eq) in dioxane (1 mL) and H2O (0.3 mL) was added Pd(dppf)Cl2 (7.10 mg, 9.70 μmol, 0.1 eq) under N2 atmosphere. The mixture was degassed and purged with N2 for 3 times, and then the mixture was stirred at 80° C. for 1 h under N2 atmosphere. The reaction mixture was diluted with H2O (10 mL) and extracted with EtOAc (10 mL*3). The combined organic layers were dried over anhydrous Na2SO4, filtered and concentrated under reduced pressure to give a residue. The residue was purified by prep-HPLC (column: Phenomenex Luna C18 150*25 mm*10 um; mobile phase:[water (TFA)-ACN]; gradient: 22%-52% B over 9 min). Compound 5-[3-(2,2-dimethyl-3-oxo-piperazine-1-carbonyl)-7-methoxy-1-(3-thienyl)-4,5-dihydrobenzo[g]indazol-8-yl]pyridine-3-carboxamide (13.23 mg, 19.73 μmol, 20.34% yield, 100% purity, TFA) was obtained as a yellow solid. LCMS (ESI): m/z[M+H] calcd for C29H28N6O4S: 556.19; found: 557.2. 1H NMR: (400 MHz, METHANOL-d4) δ=8.99 (s, 1H), 8.76 (s, 1H), 8.46 (br d, J=1.8 Hz, 1H), 7.73-7.70 (m, 1H), 7.68 (dd, J=3.3, 5.0 Hz, 1H), 7.28 (dd, J=1.2, 5.1 Hz, 1H), 7.23 (s, 1H), 6.85 (s, 1H), 3.95-3.88 (m, 5H), 3.45-3.39 (m, 2H), 3.13-3.06 (m, 2H), 2.92-2.85 (m, 2H), 1.84 (s, 6H). FIG. 171 shows the nuclear magnetic resonance of Compound 12-23.

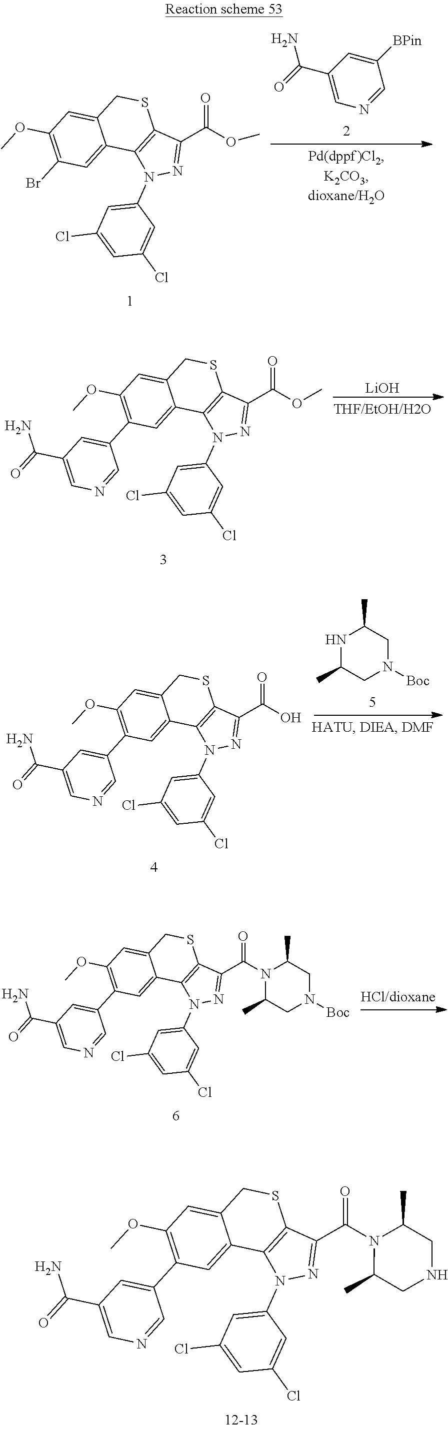

### Synthesis of Compound 12-13

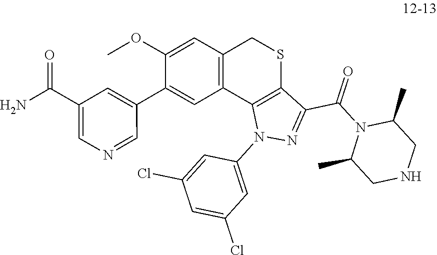 [SMILES: COc1cc2c(cc1-c1cncc(C(N)=O)c1)-c1c(c(C(=O)N3[C@H](C)CNC[C@@H]3C)nn1-c1cc(Cl)cc(Cl)c1)SC2]

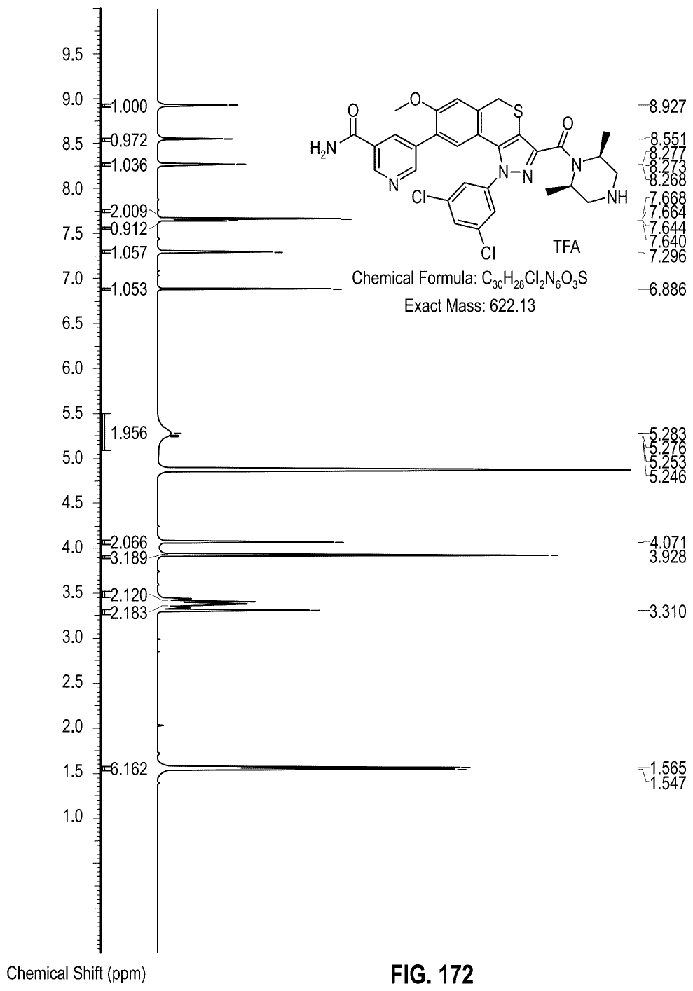

Compound 12-13 was synthesized by a procedure similar to Reaction scheme 53. LCMS (ESI): m/z[M+H] calcd for C30H29Cl2N6O3S: 623.13; found: 623.2. 1H NMR (400 MHz, METHANOL-d4) δ=8.93 (s, 1H), 8.55 (s, 1H), 8.29-8.25 (m, 1H), 7.67 (d, J=1.3 Hz, 2H), 7.64 (d, J=1.5 Hz, 1H), 7.30 (s, 1H), 6.89 (s, 1H), 5.26 (br dd, J=2.7, 12.2 Hz, 2H), 4.07 (s, 2H), 3.93 (s, 3H), 3.45-3.40 (m, 2H), 3.39-3.33 (m, 2H), 1.56 (d, J=7.2 Hz, 6H). FIG. 172 shows the nuclear magnetic resonance of Compound 12-13.

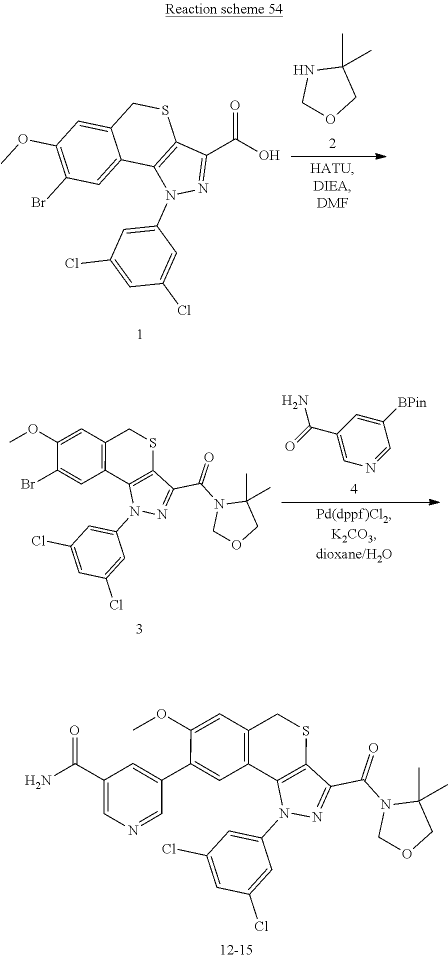

### Synthesis of Compound 12-15

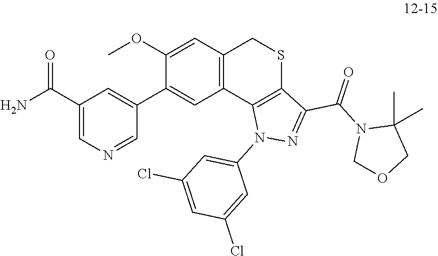 [SMILES: COc1cc2c(cc1-c1cncc(C(N)=O)c1)-c1c(c(C(=O)N3COCC3(C)C)nn1-c1cc(Cl)cc(Cl)c1)SC2]

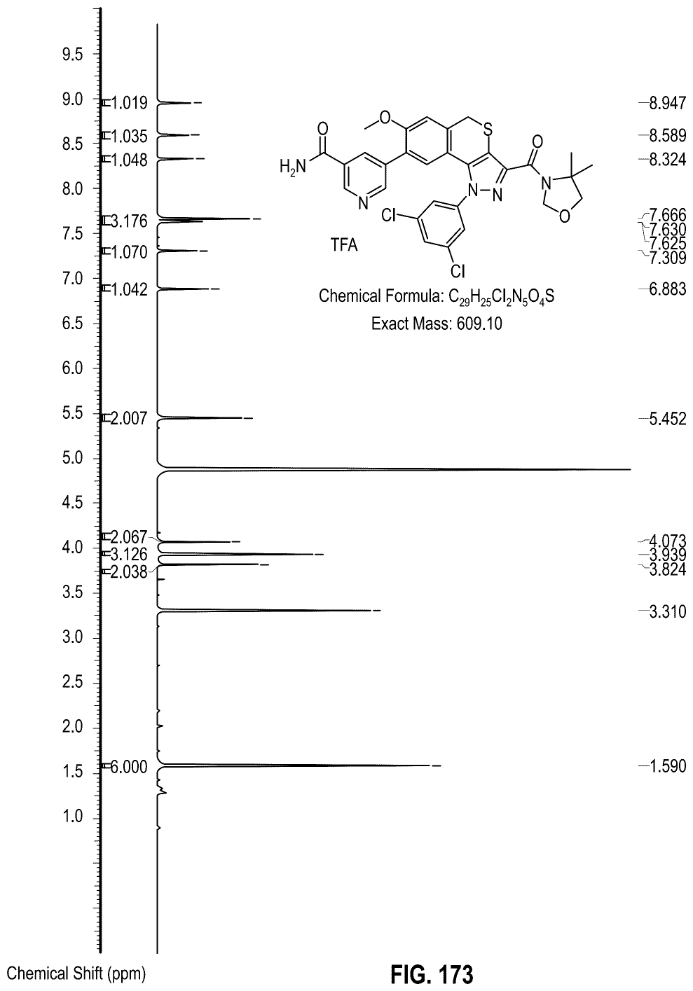

Compound 12-15 was synthesized by a procedure similar to Reaction scheme 54. LCMS (ESI): m/z[M+H] calcd for C29H26Cl2N5O4S: 610.10; found: 610.1. 1H NMR (400 MHz, METHANOL-d4) δ=8.95 (d, J=1.1 Hz, 1H), 8.59 (s, 1H), 8.32 (s, 1H), 7.70-7.60 (m, 3H), 7.31 (s, 1H), 6.88 (s, 1H), 5.45 (s, 2H), 4.07 (s, 2H), 3.94 (s, 3H), 3.82 (s, 2H), 1.59 (s, 6H). FIG. 173 shows the nuclear magnetic resonance of Compound 12-15.

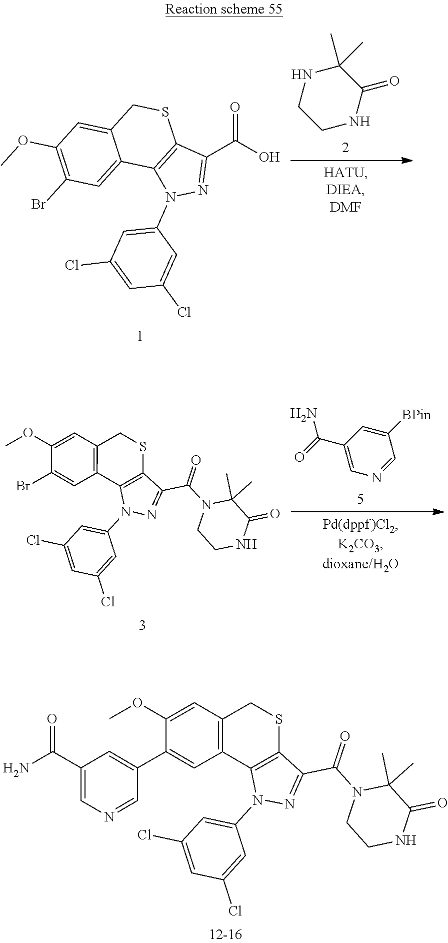

### Synthesis of Compound 12-16

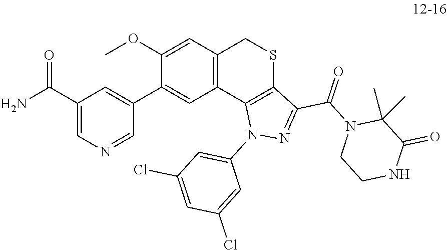 [SMILES: COc1cc2c(cc1-c1cncc(C(N)=O)c1)-c1c(c(C(=O)N3CCNC(=O)C3(C)C)nn1-c1cc(Cl)cc(Cl)c1)SC2]

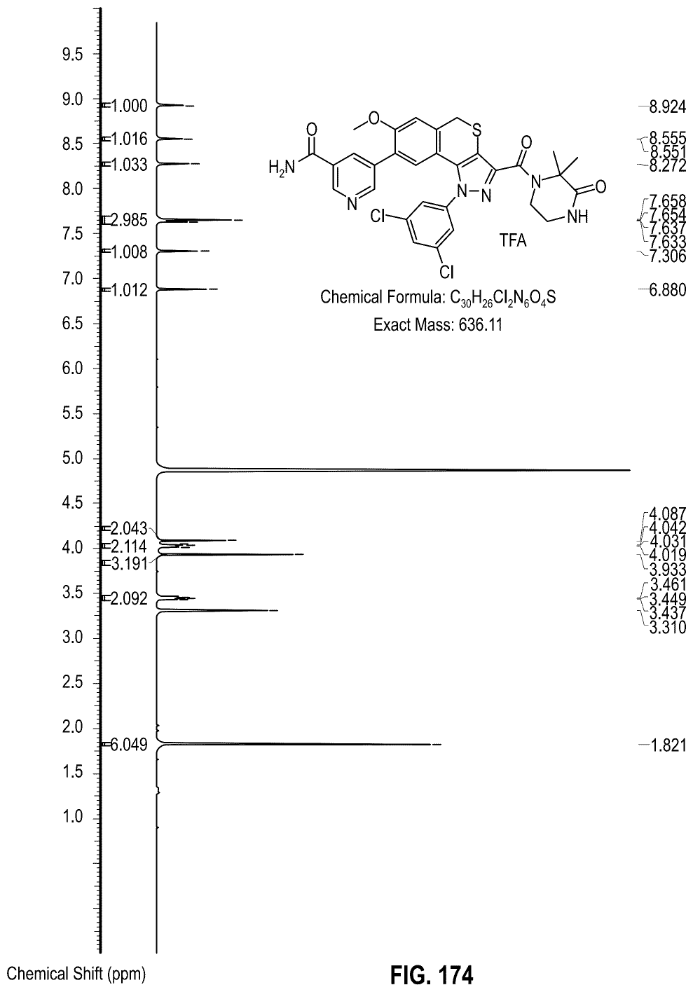

Compound 12-15 was synthesized by a procedure similar to Reaction scheme 55. LCMS (ESI): m/z[M+H] calcd for C30H27Cl2N6O4S: 637.11; found: 637.1. 1H NMR (400 MHz, METHANOL-d4) δ=8.93 (d, J=1.5 Hz, 1H), 8.55 (d, J=1.5 Hz, 1H), 8.27 (s, 1H), 7.69-7.60 (m, 3H), 7.31 (s, 1H), 6.88 (s, 1H), 4.09 (s, 2H), 4.06-4.00 (m, 2H), 3.93 (s, 3H), 3.48-3.42 (m, 2H), 1.82 (s, 6H). FIG. 174 shows the nuclear magnetic resonance of Compound 12-16.

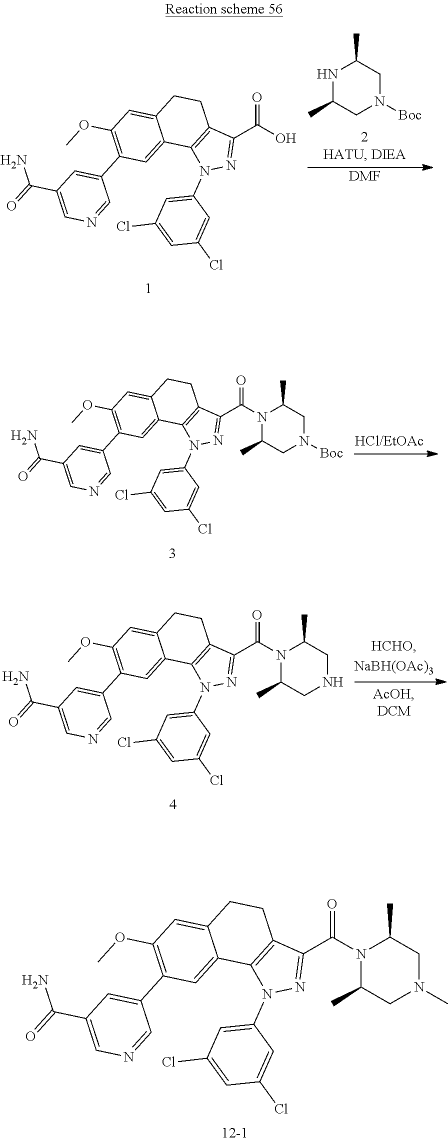

### Synthesis of Compound 12-1

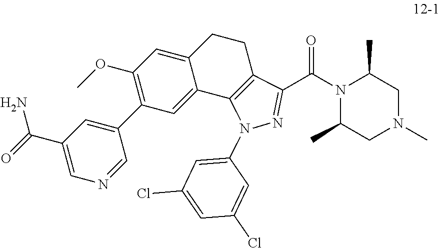 [SMILES: COc1cc2c(cc1-c1cncc(C(N)=O)c1)-c1c(c(C(=O)N3[C@H](C)CN(C)C[C@@H]3C)nn1-c1cc(Cl)cc(Cl)c1)CC2]

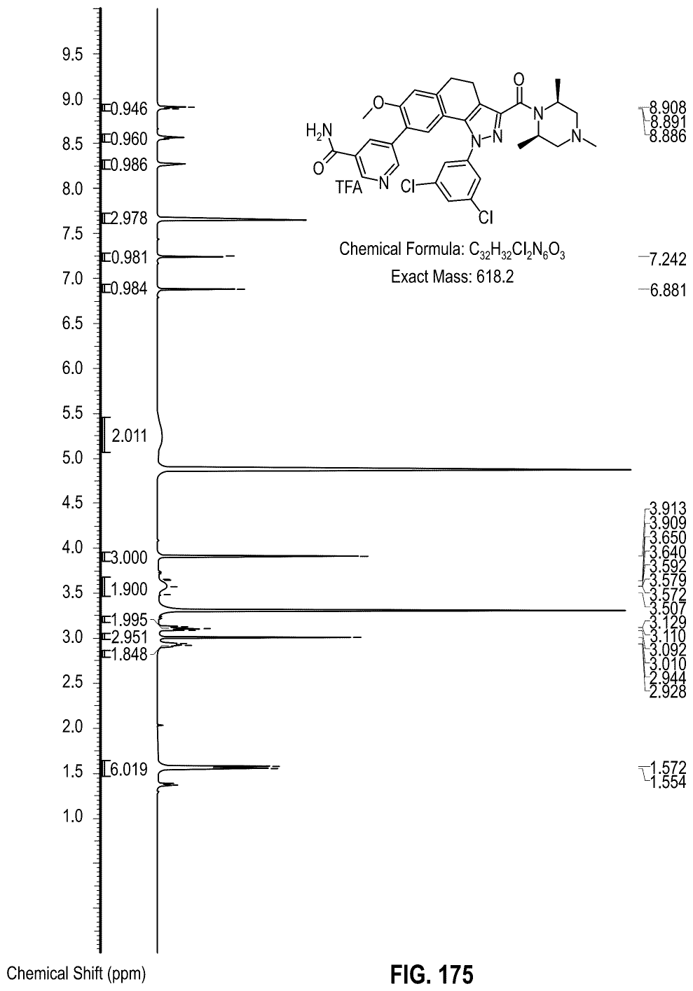

Compound 12-1 was synthesized by a procedure similar to Reaction scheme 56. LCMS (ESI): m/z[M+H] calcd for C34H33N6O5Cl2F3: 619.19; found: 619.3. 1H NMR (400 MHz, METHANOL-d4) δ=8.94-8.86 (m, 1H), 8.60-8.51 (m, 1H), 8.32-8.22 (m, 1H), 7.7 2-7.63 (m, 3H), 7.24 (s, 1H), 6.88 (s, 1H), 5.47-5.06 (m, 2H), 3.96-3.85 (m, 3H), 3.67-3.48 (m, 2H), 3.15-3.08 (m, 2H), 3.01 (s, 3H), 2.94 (br d, J=6.38 Hz, 2H), 1.56 (br d, J=7.13 Hz, 6H). FIG. 175 shows the nuclear magnetic resonance of Compound 12-1.

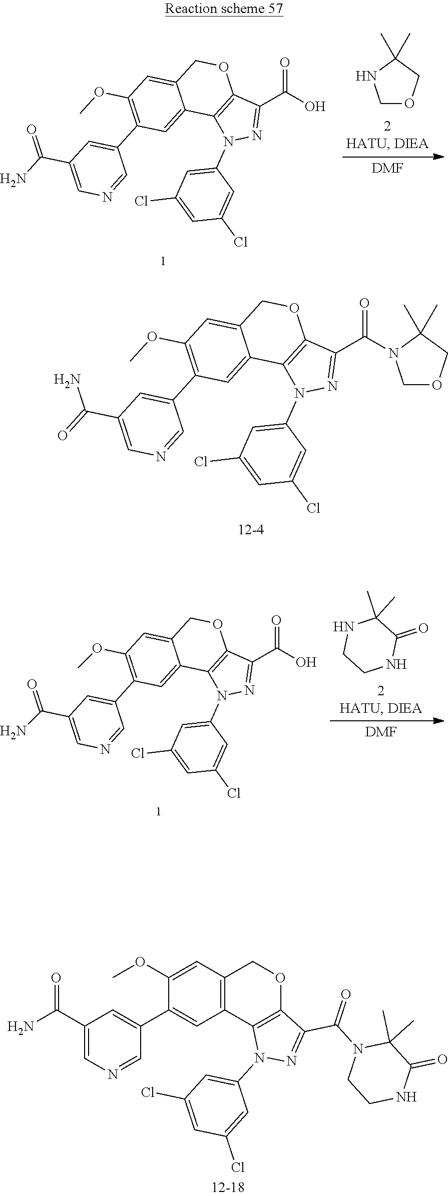

### Synthesis of Compound 12-4

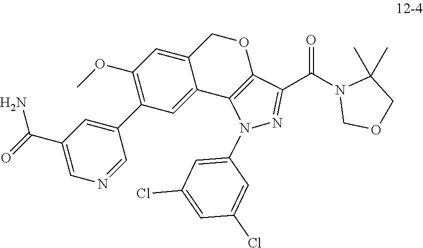 [SMILES: COc1cc2c(cc1-c1cncc(C(N)=O)c1)-c1c(c(C(=O)N3COCC3(C)C)nn1-c1cc(Cl)cc(Cl)c1)OC2]

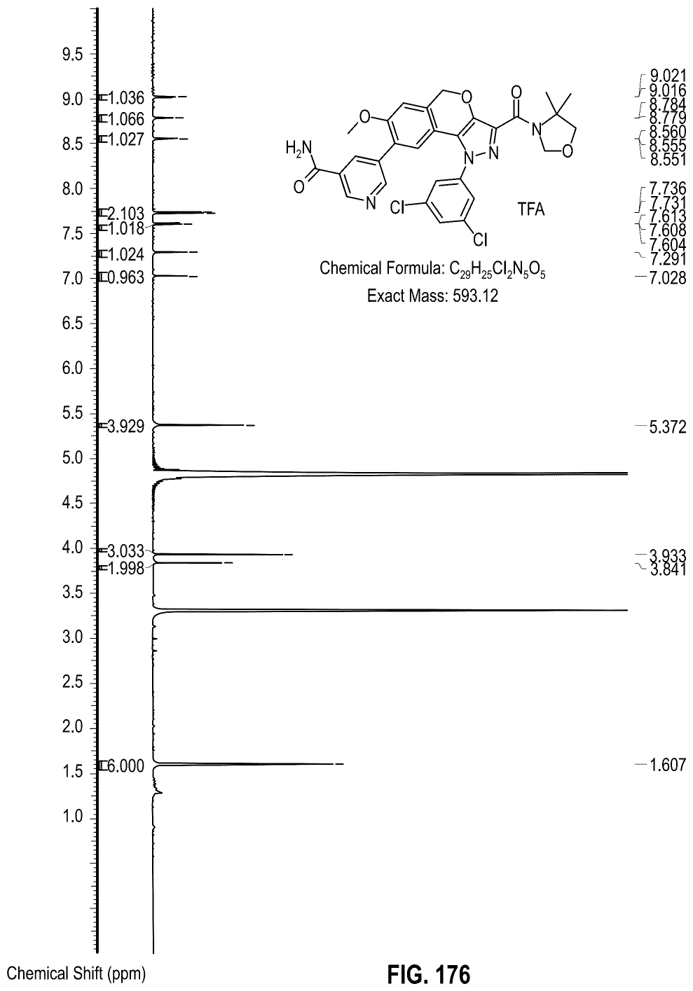

Compound 12-4 was synthesized by a procedure similar to Reaction scheme 57. LCMS (ESI): m/z[M+H] calcd for C29H26Cl2N5O5: 594.12; found: 594.1. 1H NMR (400 MHz, METHANOL-d4) δ=9.02 (d, J=1.9 Hz, 1H), 8.78 (d, J=1.8 Hz, 1H), 8.56 (t, J=1.9 Hz, 1H), 7.73 (d, J=1.8 Hz, 2H), 7.61 (t, J=1.8 Hz, 1H), 7.29 (s, 1H), 7.03 (s, 1H), 5.37 (s, 4H), 3.93 (s, 3H), 3.84 (s, 2H), 1.61 (s, 6H). FIG. 176 shows the nuclear magnetic resonance of Compound 12-4.

### Synthesis of Compound 12-18

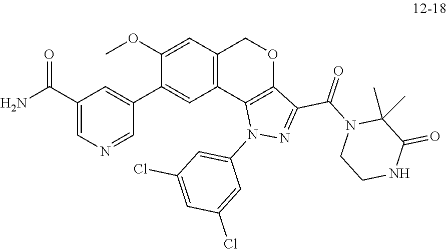 [SMILES: COc1cc2c(cc1-c1cncc(C(N)=O)c1)-c1c(c(C(=O)N3CCNC(=O)C3(C)C)nn1-c1cc(Cl)cc(Cl)c1)OC2]

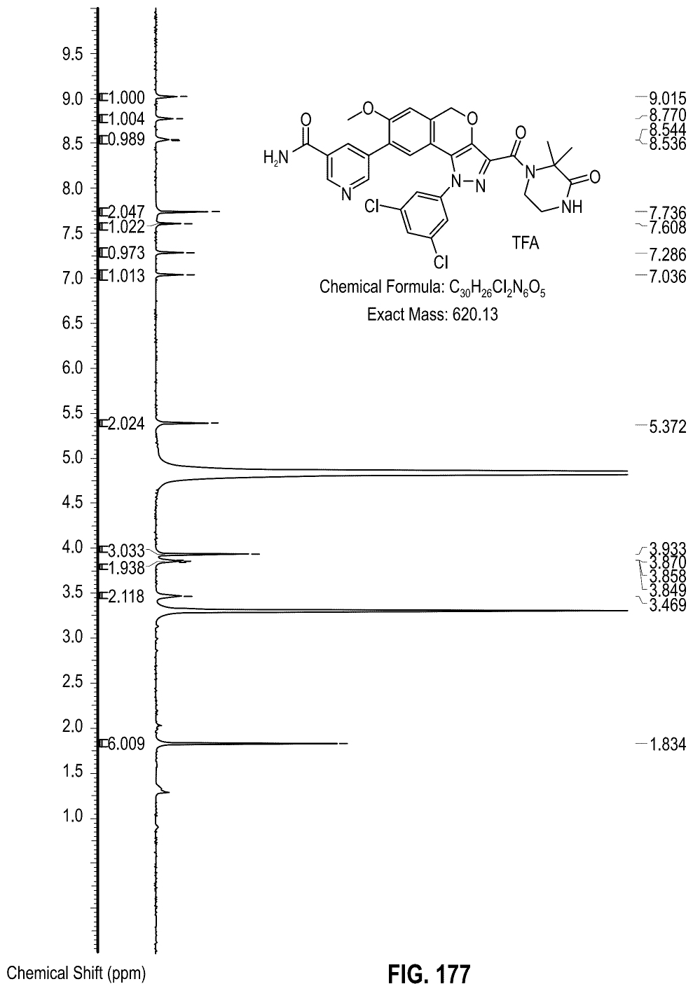

Compound 12-18 was synthesized by a procedure similar to Reaction scheme 57. LCMS (ESI):m z[M+H] calcd for C₃₄H₃₃N₆O₅Cl₂F₃: 619.19; found: 619.3. ¹H NMR (400 MHz, METHANOL-d₄) δ=8.94-8.86 (m, 1H), 8.60-8.51 (m, 1H), 8.32-8.22 (m, 1H), 7.7 2-7.63 (m, 3H), 7.24 (s, 1H), 6.88 (s, 1H), 5.47-5.06 (m, 2H), 3.96-3.85 (m, 3H), 3.67-3.48 (m, 2H), 3.15-3.08 (m, 2H), 3.01 (s, 3H), 2.94 (br d, J=6.38 Hz, 2H), 1.56 (br d, J=7.13 Hz, 6H). FIG. 177 shows the nuclear magnetic resonance of Compound 12-18.

### Synthesis of Compound 12-19

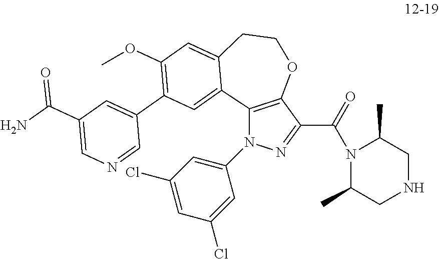 [SMILES: COc1cc2c(cc1-c1cncc(C(N)=O)c1)-c1c(c(C(=O)N3[C@H](C)CNC[C@@H]3C)nn1-c1cc(Cl)cc(Cl)c1)OCC2]

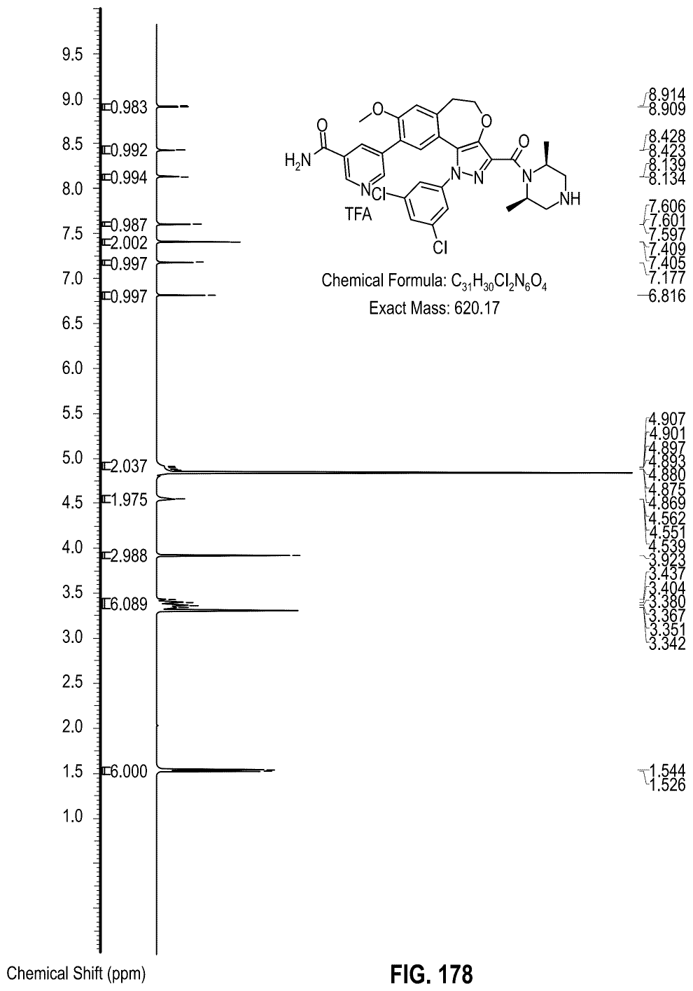

Compound 12-19 was synthesized by a procedure similar to Reaction scheme 57. LCMS (ESI): m/z[M+H] calcd for C₃₄H₃₃N₆O₅Cl₂F₃: 619.19; found: 619.3. ¹H NMR (400 MHz, METHANOL-d₄) δ=8.94-8.86 (m, 1H), 8.60-8.51 (m, 1H), 8.32-8.22 (m, 1H), 7.7 2-7.63 (m, 3H), 7.24 (s, 1H), 6.88 (s, 1H), 5.47-5.06 (m, 2H), 3.96-3.85 (m, 3H), 3.67-3.48 (m, 2H), 3.15-3.08 (m, 2H), 3.01 (s, 3H), 2.94 (br d, J=6.38 Hz, 2H), 1.56 (br d, J=7.13 Hz, 6H). FIG. 178 shows the nuclear magnetic resonance of Compound 12-19.

### Example 13

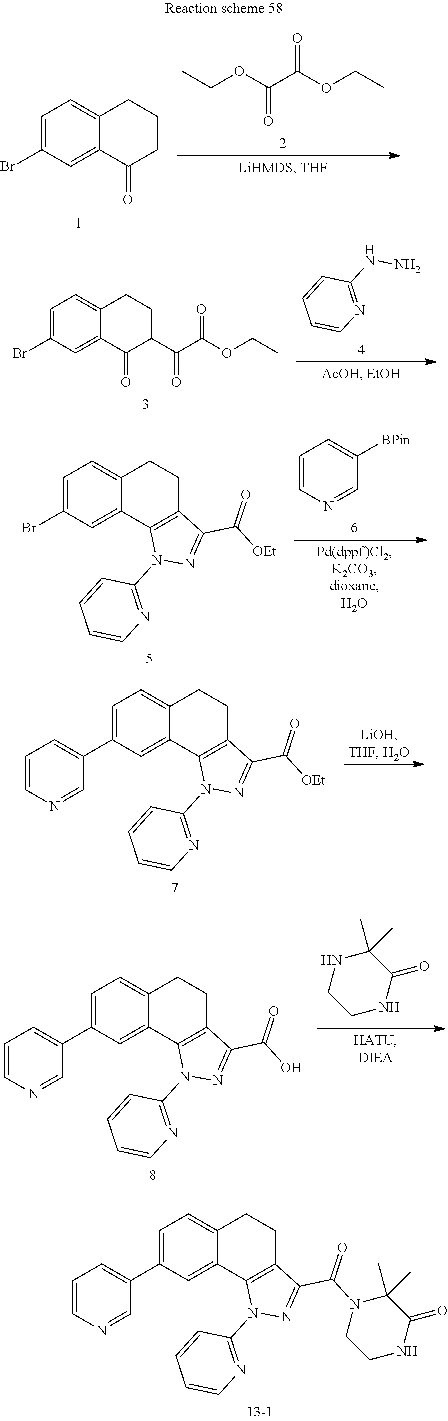

### Synthesis of ethyl 2-(7-bromo-1-oxo-1,2,3,4-tetrahydronaphthalen-2-yl)-2-oxoacetatecarboxylate

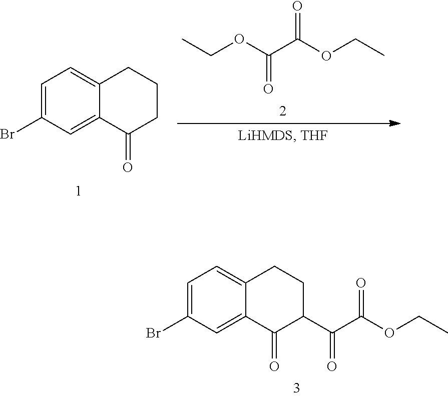

To a solution of 7-bromotetralin-1-one (3 g, 13.33 mmol, 1 eq) in THF (20 mL) was added dropwise LiHMDS (1 M, 19.99 mL, 1.5 eq) at −40° C. over 10 min. After addition, the mixture was stirred at this temperature for 0.5 hr, and then diethyl oxalate (2.53 g, 17.33 mmol, 2.37 mL, 1.3 eq) in THF (10 mL) was added dropwise at −40° C. The resulting mixture was stirred at 0° C. for 2 hr. The reaction mixture was quench with 1N HCl (30 mL) and poured into ice-water (100 mL). The aqueous phase was extracted with ethyl acetate (100 mL*3). The combined organic phase was washed with brine (200 mL), dried with anhydrous Na2SO4, filtered and concentrated in vacuum. The crude product was purified by reversed-phase HPLC (0.1% FA condition). Compound ethyl 2-(7-bromo-1-oxo-tetralin-2-yl)-2-oxo-acetate (2.5 g, 7.69 mmol, 57.69% yield) was obtained as a yellow oil. 1H NMR (400 MHz, DMSO-d6) δ=7.90 (br s, 1H), 7.71 (br d, J=7.9 Hz, 1H), 7.35-7.29 (m, 1H), 4.29 (br d, J=6.6 Hz, 2H), 2.87-2.79 (m, 2H), 2.71-2.63 (m, 2H), 1.29 (br t, J=7.0 Hz, 3H).

### Synthesis of ethyl 8-bromo-1-(pyridin-2-yl)-4,5-dihydro-1H-benzo[g]indazole-3-carboxylate

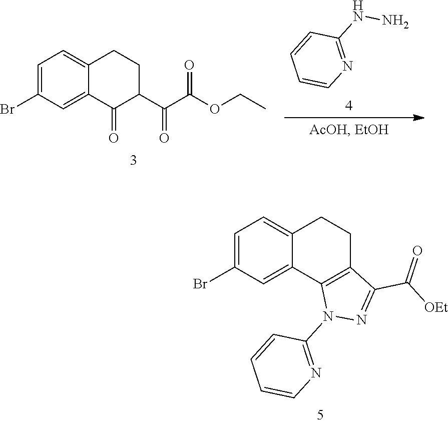

A mixture of ethyl 2-(7-bromo-1-oxo-tetralin-2-yl)-2-oxo-acetate (0.3 g, 922.64 μmol, 1 eq), 2-pyridylhydrazine (100.69 mg, 922.64 μmol, 1 eq) and AcOH (554.06 mg, 9.23 mmol, 528.18 μL, 10 eq) in EtOH (5 mL) was stirred at 80° C. for 16 hr under N₂ atmosphere. The residue was concentrated. The residue was purified by flash silica gel chromatography (ISCO®; 12 g SepaFlash® Silica Flash Column, Eluent of 0˜20% Ethyl acetate/Petroleum ethergradient @50 mL/min). Compound ethyl 8-bromo-1-(2-pyridyl)-4,5-dihydrobenzo[g]indazole-3-carboxylate (260 mg, 652.85 μmol, 70.76% yield) was obtained as a white solid. LCMS (ESI): m/z[M+H] calcd for C19H17BrN3O2: 398.14; found: 397.9.

### Synthesis of ethyl 1-(pyridin-2-yl)-8-(pyridin-3-yl)-4,5-dihydro-1H-benzo[g]indazole-3-carboxylate

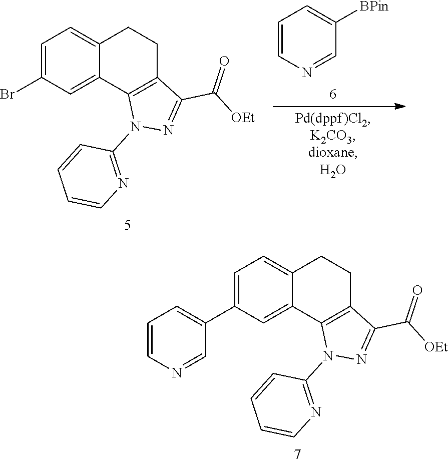

ethyl 8-bromo-1-(2-pyridyl)-4,5-dihydrobenzo[g]indazole-3-carboxylate (180 mg, 451.97 μmol, 1 eq), 3-pyridylboronic acid (66.67 mg, 542.37 μmol, 1.2 eq), K2CO3 (124.93 mg, 903.95 μmol, 2 eq) and Pd(dppf)Cl2 (66.14 mg, 90.39 μmol, 0.2 eq) in dioxane (4 mL) and H2O (0.4 mL) was degassed and then heated to 60° C. for 16 hours under N2. The reaction mixture was diluted with Ethyl acetate (20 mL) and then filtered. The filtrate was concentrated. The residue was purified by flash silica gel chromatography (ISCO®; 10 g SepaFlash® Silica Flash Column, Eluent of 0˜100% Ethyl acetate/Petroleum ethergradient @40 mL/min). Compound ethyl 1-(2-pyridyl)-8-(3-pyridyl)-4,5-dihydrobenzo[g]indazole-3-carboxylate (110 mg, 277.47 μmol, 61.39% yield) was obtained as a white solid. LCMS (ESI): m/z[M+H] calcd for C24H21N4O2: 397.16; found: 397.1.

### Synthesis of 1-(pyridin-2-yl)-8-(pyridin-3-yl)-4,5-dihydro-1H-benzo[g]indazole-3-carboxylic acid

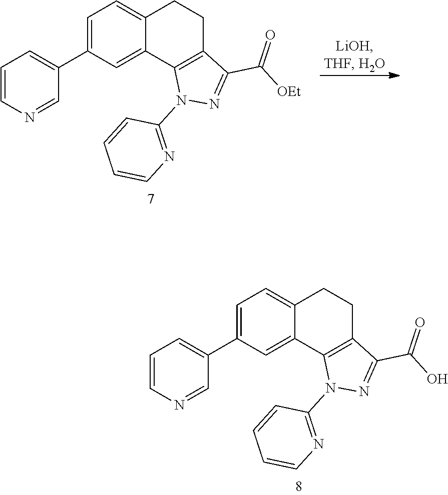

A mixture of ethyl 1-(2-pyridyl)-8-(3-pyridyl)-4,5-dihydrobenzo[g]indazole-3-carboxylate (110 mg, 277.47 μmol, 1 eq) and LiOH. H2O (46.57 mg, 1.11 mmol, 4 eq) in EtOH (2 mL) was stirred at 25° C. for 2 hr under N₂ atmosphere. The reaction mixture was poured into ice-water (20 mL) and neutralized with 1N HCl. The aqueous phase was extracted with ethyl acetate (10 mL*3). The combined organic phase was washed with brine (30 mL), dried with anhydrous Na2SO4, filtered and concentrated in vacuum. The crude product 1-(2-pyridyl)-8-(3-pyridyl)-4,5-dihydrobenzo[g]indazole-3-carboxylic acid (95 mg, crude) as a yellow oil was used into the next step without further purification.

### Synthesis of Compound 13-1

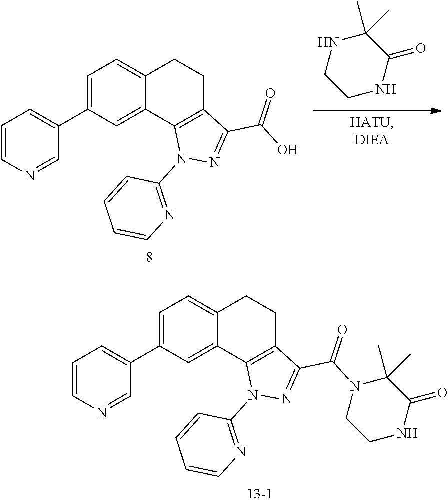

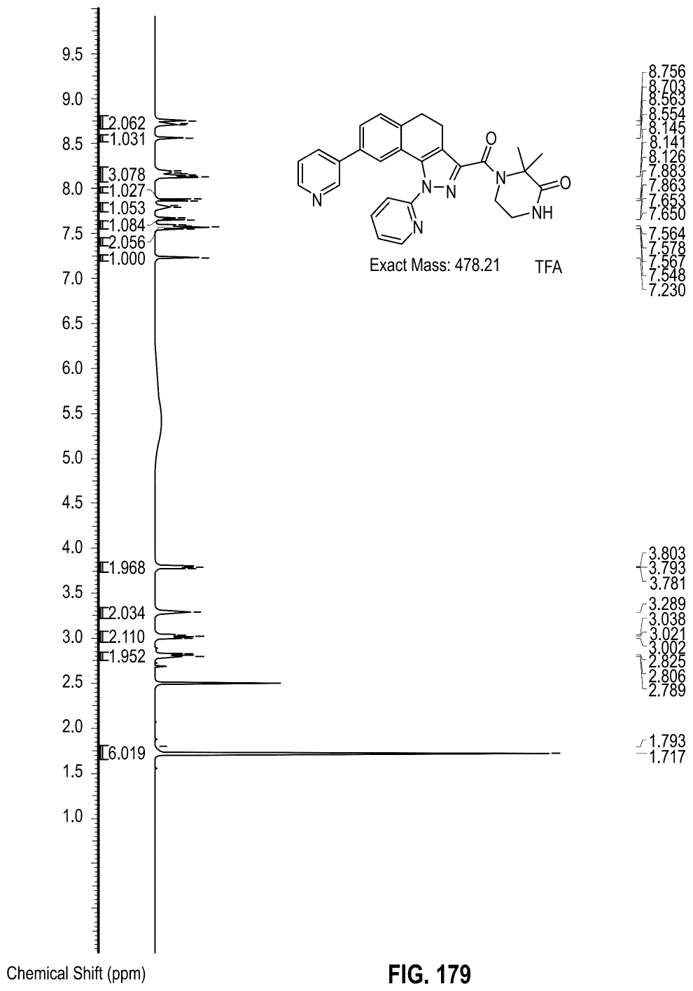

A mixture of 1-(2-pyridyl)-8-(3-pyridyl)-4,5-dihydrobenzo[g]indazole-3-carboxylic acid (60 mg, 162.87 μmol, 1 eq), 3,3-dimethylpiperazin-2-one (22.96 mg, 179.16 μmol, 1.1 eq), HATU (92.89 mg, 244.31 μmol, 1.5 eq), DIEA (63.15 mg, 488.62 μmol, 85.11 μL, 3 eq) in DMF (1 mL), and then the mixture was stirred at 25° C. for 2 hr. The pH of the reaction mixture was adjusted to 7 with 1 M HCl. The residue was purified by prep-HPLC (TFA condition;column: Phenomenex Luna C18 150*25 mm*10 um;mobile phase:[water(TFA)-ACN];gradient: 12%-42% B over 9 min). Compound 3,3-dimethyl-4-[1-(2-pyridyl)-8-(3-pyridyl)-4,5-dihydrobenzo[g]indazole-3-carbonyl]piperazin-2-one (23.5 mg, 39.26 μmol, 24.11% yield, 99% purity, TFA) was obtained as an off-white solid. LCMS (ESI): m/z[M+H] calcd for C27H25N7O2: 479.21; found: 479.3. 1H NMR (400 MHz, DMSO-d6) δ=8.81-8.66 (m, 2H), 8.56 d, J=3.6 Hz, 1H), 8.24-8.08 (m, 3H), 7.87 (d, J=8.0 Hz, 1H), 7.80 (m, 1H), 7.66 (dd, J=1.6, 8.0 Hz, 1H), 7.61-7.53 (m, 2H), 7.23 (s, 1H), 3.85-3.74 (m, 2H), 3.29 (s, 2H), 3.07-2.96 (m, 2H), 2.85-2.76 (m, 2H), 1.72 (s, 6H). FIG. 179 shows the nuclear magnetic resonance of Compound 13-1.

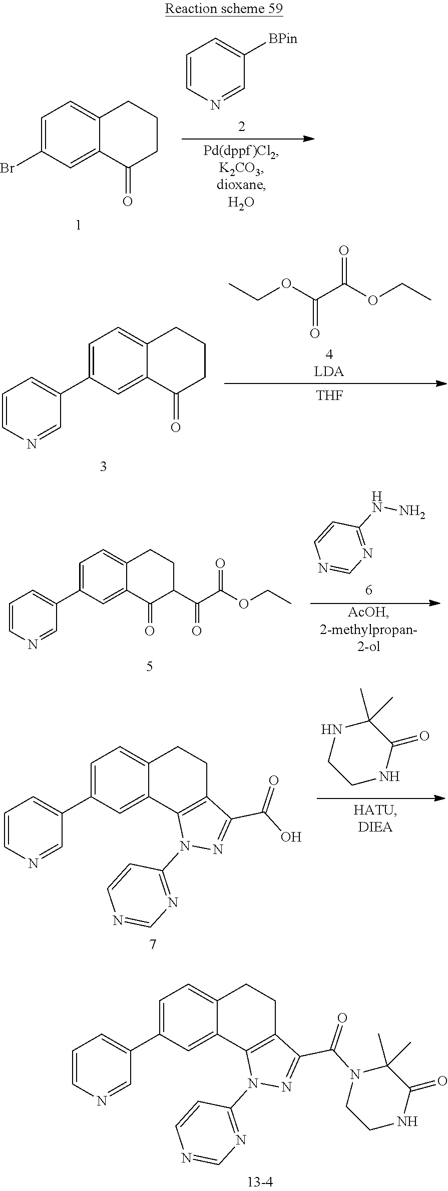

### Synthesis of 7-(pyridin-3-yl)-3,4-dihydronaphthalen-1(2H)-one-carboxylate

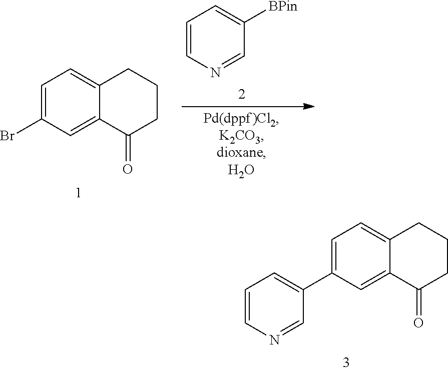

A mixture of 7-bromotetralin-1-one (5 g, 22.21 mmol, 1 eq), 3-pyridylboronic acid (3.28 g, 26.66 mmol, 1.2 eq), K2CO3 (6.14 g, 44.43 mmol, 2 eq), Pd(dppf)Cl2 (3.25 g, 4.44 mmol, 0.2 eq) and in dioxane (50 mL) and H2O (5 mL) was degassed and purged with N2 for 3 times, and then the mixture was stirred at 60° C. for 16 hr under N2 atmosphere. The mixture was filtered and concentrated under reduced pressure to give a residue. The residue was purified by flash silica gel chromatography (ISCO®; 80 g SepaFlash® Silica Flash Column, Eluent of 50˜40% Ethyl acetate/Petroleum ethergradient @100 mL/min). Compound 7-(3-pyridyl)tetralin-1-one (4.2 g, 18.81 mmol, 84.68% yield) was obtained as a light yellow solid. 1H NMR (400 MHz, DMSO-d6) δ=8.87 (d, J=2.0 Hz, 1H), 8.58 (d, J=4.4 Hz, 1H), 8.14-8.02 (m, 2H), 7.90 (dd, J=1.6, 8.0 Hz, 1H), 7.53-7.43 (m, 2H), 2.98 (t, J=6.0 Hz, 2H), 2.64 (t, J=6.8 Hz, 2H), 2.06 (m, 2H).

### Synthesis of ethyl 2-oxo-2-(1-oxo-7-(pyridin-3-yl)-1,2,3,4-tetrahydronaphthalen-2-yl)acetate

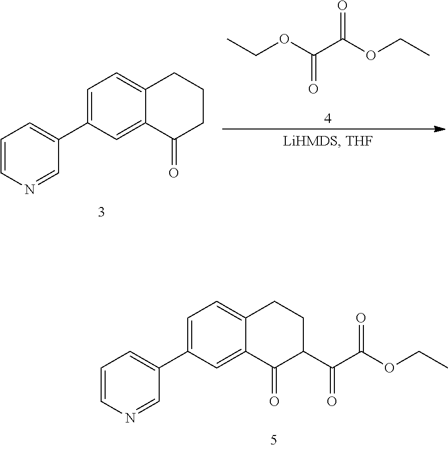

To a solution of 7-(3-pyridyl)tetralin-1-one (1 g, 4.48 mmol 1 eq) in THF (7 mL) was added dropwise LiHMDS (1 M, 6.72 mL, 1.5 eq) at −40° C. over 10 min/hr. After addition, the mixture was stirred at this temperature for 0.5 hr, and then diethyl oxalate (850.91 mg, 5.82 mmol, 795.25 μL, 1.3 eq) in THF (3 mL) was added dropwise at −40° C. The resulting mixture was stirred at 0° C. for 2 hr. The reaction mixture was quench with 1N HCl (15 mL) and poured into ice-water (50 mL). The aqueous phase was extracted with ethyl acetate (50 mL*3).The combined organic phase was washed with brine (50 mL), dried with anhydrous Na₂SO₄, filtered and concentrated in vacuum. The crude product was filtered and concentrated under reduced pressure to give a residue. The residue was triturated with 2-methoxy-2-methyl-propane at 25° C. for 5 min. Compound ethyl 2-oxo-2-[1-oxo-7-(3-pyridyl)tetralin-2-yl]acetate (340 mg, crude) was obtained as a light yellow solid.

### Synthesis of 8-(pyridin-3-yl)-1-(pyrimidin-4-yl)-4,5-dihydro-1H-benzo[g]indazole-3-carboxylic acid

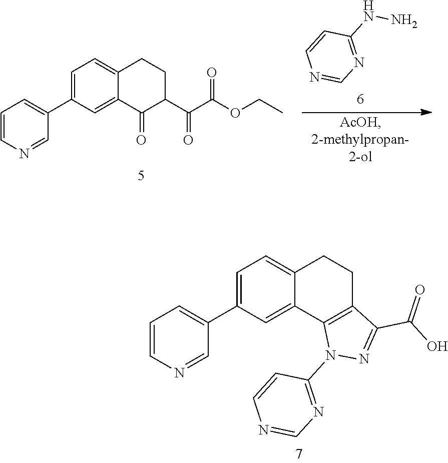

A mixture of ethyl 2-oxo-2-[1-oxo-7-(3-pyridyl)tetralin-2-yl]acetate (300 mg, 927.81 μmol, 1 eq), pyrimidin-4-ylhydrazine (122.60 mg, 1.11 mmol, 1.2 eq), AcOH (524.50 mg, 8.73 mmol, 0.5 mL, 9.41 eq) in 2-methylpropan-2-ol (0.5 mL), and then the mixture was stirred at 100° C. for 12 hr. The pH of the reaction mixture was adjusted to 9-10 with NH3·H2O,and filtered and concentrated under reduced pressure to give a residue. The residue was purified by prep-HPLC (column: Phenomenex luna C18 150*25 mm*10 um;mobile phase:[water(TFA)-ACN];gradient:8%-38% B over 15 min). Compound ethyl 8-(3-pyridyl)-1-pyrimidin-4-yl-4,5-dihydrobenzo[g]indazole-3-carboxylate (220 mg, 498.20 μmol, 53.70% yield, 90% purity) was obtained as a yellow solid. 1H NMR (400 MHz, DMSO-d6) δ=9.13 (s, 1H), 9.05-8.99 (m, 1H), 8.70 (d, J=2.0 Hz, 1H), 8.52 (dd, J=1.2, 4.8 Hz, 1H), 8.02 (d, J=5.6 Hz, 1H), 7.89 (td, J=2.0, 8.0 Hz, 1H), 7.63-7.57 (m, 2H), 7.49 (d, J=7.6 Hz, 1H), 7.44 (dd, J=4.8, 8.0 Hz, 1H), 2.95 (s, 4H).

### Synthesis of Compound 13-4

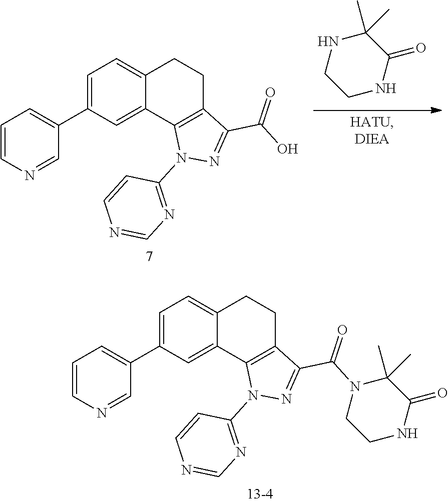

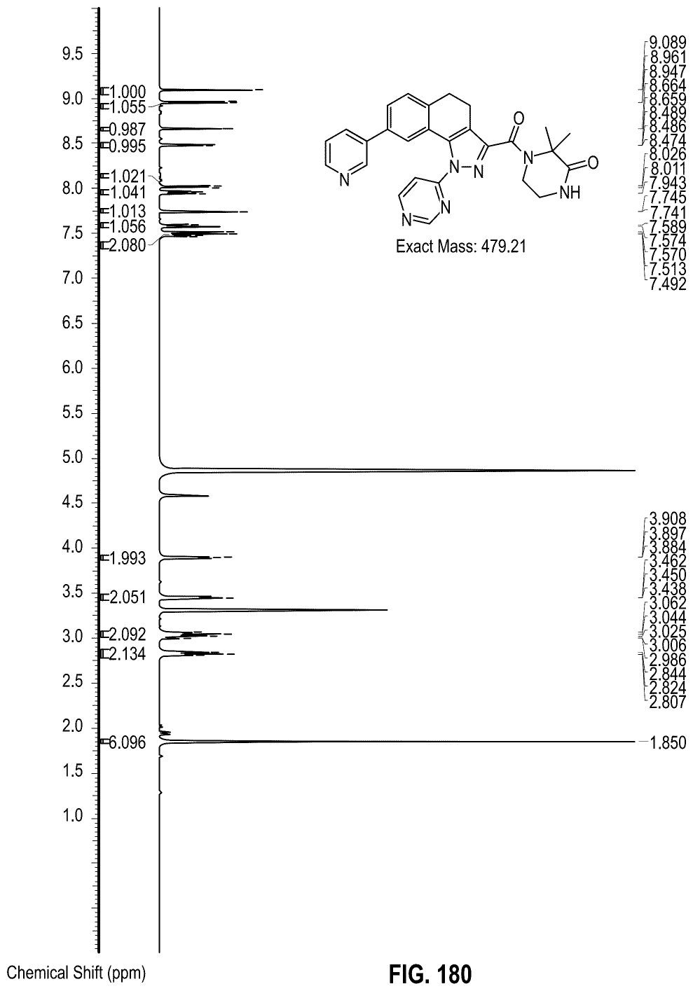

To a solution of 8-(3-pyridyl)-1-pyrimidin-4-yl-4,5-dihydrobenzo[g]indazole-3-carboxylic acid (50 mg, 135.36 μmol, 1 eq) in DMF (1 mL) was added DIEA (52.48 mg, 406.09 μmol, 70.73 μL, 3 eq) and HATU (77.20 mg, 203.05 μmol, 1.5 eq). The mixture was stirred at 25° C. for 0.5 hr.then,3,3-dimethylpiperazin-2-one (20.82 mg, 162.44 μmol, 1.2 eq) was added. The mixture was stirred at 25° C. for 1.5 hr. The residue was purified by prep-HPLC (column: Waters Xbridge 150*25 mm*5 um;mobile phase:[water (ammonia hydroxide v/v)-ACN];gradient:10%-40% B over 10 min). Compound 3,3-dimethyl-4-[8-(3-pyridyl)-1-pyrimidin-4-yl-4,5-dihydrobenzo[g]indazole-3-carbonyl]piperazin-2-one (9.68 mg, 20.19 μmol, 14.91% yield) was obtained as a white solid. LCMS (ESI): m/z[M+H] calcd for C27H25N7O2: 480.21; found: 480.2. 1H NMR (400 MHz, METHANOL-d4) δ=9.09 (s, 1H), 8.95 (d, J=5.6 Hz, 1H), 8.66 (d, J=2.0 Hz, 1H), 8.48 (dd, J=1.2, 4.8 Hz, 1H), 8.02 (d, J=6.0 Hz, 1H), 7.95 (td, J=1.6, 8.0 Hz, 1H), 7.74 (d, J=1.6 Hz, 1H), 7.61-7.56 (m, 1H), 7.52-7.44 (m, 2H), 3.92-3.87 (m, 2H), 3.49-3.42 (m, 2H), 3.08-3.01 (m, 2H), 2.87-2.77 (m, 2H), 1.85 (s, 6H). FIG. 180 shows the nuclear magnetic resonance of Compound 13-4.

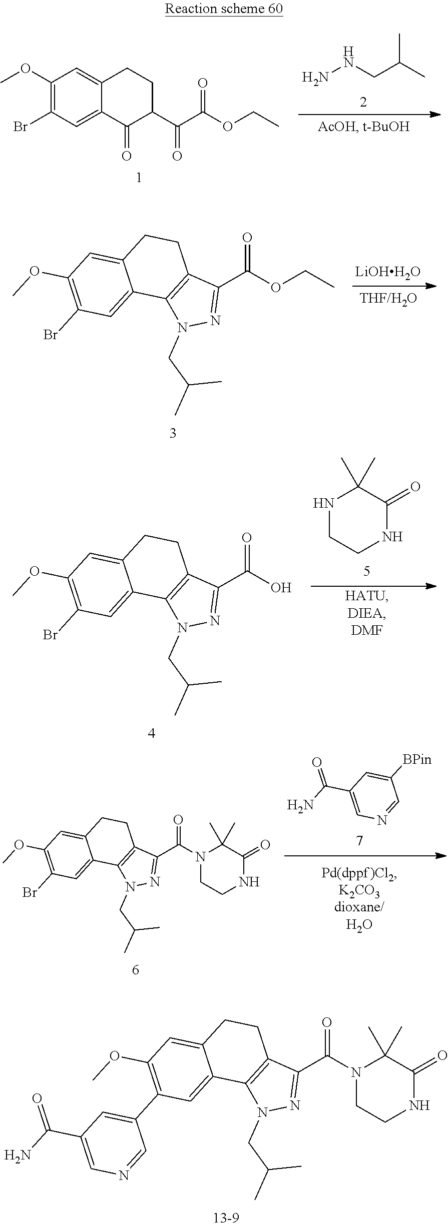

### Synthesis of ethyl 8-bromo-1-isobutyl-7-methoxy-4,5-dihydrobenzo[g]indazole-3-carboxylate

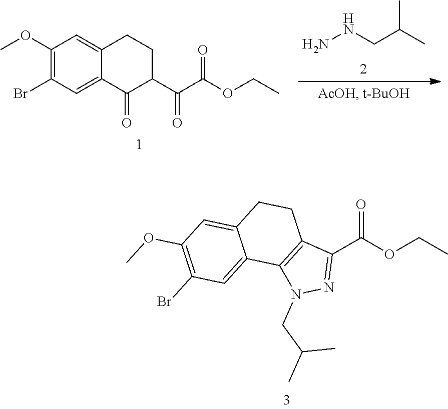

A mixture of ethyl 2-(7-bromo-6-methoxy-1-oxo-tetralin-2-yl)-2-oxo-acetate (350 mg, 985.42 μmol, 1 eq), isobutylhydrazine; hydrochloride (135.07 mg, 1.08 mmol, 1.1 eq) and AcOH (2.37 g, 39.42 mmol, 2.26 mL, 40 eq) in t-BuOH (2 mL) was stirred at 90° C. for 1 h. The reaction mixture was concentrated under reduced pressure to remove t-BuOH. The pH of the resulting mixture was adjusted to 9 with NaHCO₃under stirred. And then a lot of solid precipitated out. The mixture was filtered, the filter cake was dried under reduced pressure to give compound ethyl 8-bromo-1-isobutyl-7-methoxy-4,5-dihydrobenzo[g]indazole-3-carboxylate (460 mg, crude) was obtained as a yellow solid. LCMS (ESI): m/z[M+H] calcd for C19H23BrN2O3: 406.09; found: 407.0.

### Synthesis of 8-bromo-1-isobutyl-7-methoxy-4,5-dihydrobenzo[g]indazole-3-carboxylic acid

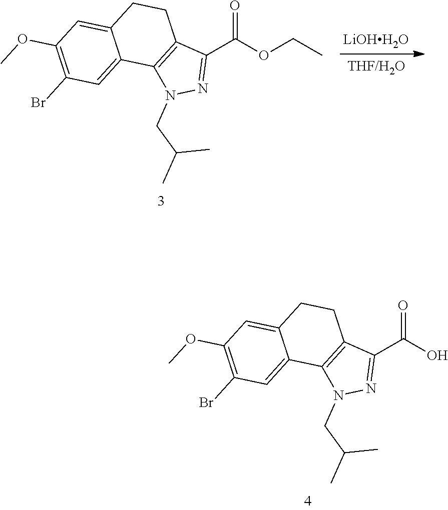

A mixture of ethyl 8-bromo-1-isobutyl-7-methoxy-4,5-dihydrobenzo[g]indazole-3-carboxylate (460 mg, 1.13 mmol, 1 eq) and LiOH. H2O (142.18 mg, 3.39 mmol, 3 eq) in EtOH (3 mL) and H2O (1 mL) was stirred at 25° C. for 1 hr. The mixture was stirred at 60° C. for 2 h. The reaction mixture was concentrated under reduced pressure to remove EtOH. The pH of the resulting mixture was adjusted to 4 with 1 N HCl, a lot of solid precipitated out. The mixture was filtered, the filter cake was dried under reduced pressure to give compound 8-bromo-1-isobutyl-7-methoxy-4,5-dihydrobenzo[g]indazole-3-carboxylic acid (360 mg, crude) was obtained as a light yellow solid. LCMS (ESI): m/z[M+H] calcd for C17H20BrN2O3: 379.06; found: 379.0.

### Synthesis of 4-(8-bromo-1-isobutyl-7-methoxy-4,5-dihydrobenzo[g]indazole-3-carbonyl)-3,3-dimethyl-piperazin-2-one

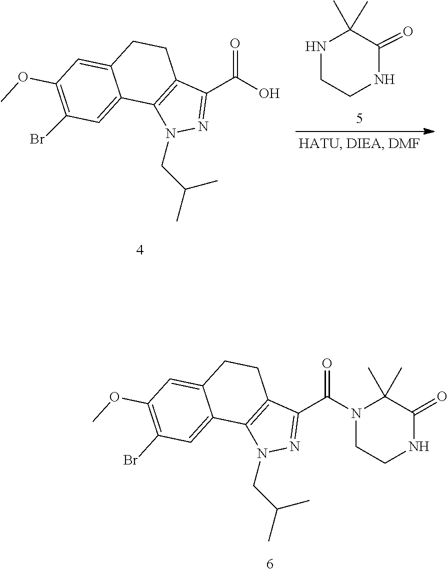

A mixture of 8-bromo-1-isobutyl-7-methoxy-4,5-dihydrobenzo[g]indazole-3-carboxylic acid (80 mg, 210.94 μmol, 1 eq), HATU (120.31 mg, 316.42 μmol, 1.5 eq) and DIEA (109.05 mg, 843.78 μmol, 146.97 μL, 4 eq) in DMF (1.5 mL) was stirred at 25° C. for 15 min. To the mixture was added 3, 3-dimethylpiperazin-2-one (35.15 mg, 274.23 μmol, 1.3 eq). The mixture was stirred at 25° C. for 1 h. The reaction mixture was diluted with H2O (20 mL) and a lot of solid precipitated, the resulting mixture was filtered, the filter cake was dried under reduced pressure to give compound 4-(8-bromo-1-isobutyl-7-methoxy-4,5-dihydrobenzo[g]indazole-3-carbonyl)-3,3-dimethyl-piperazin-2-one (100 mg, crude) was obtained as a white solid.

### Synthesis of Compound 13-9

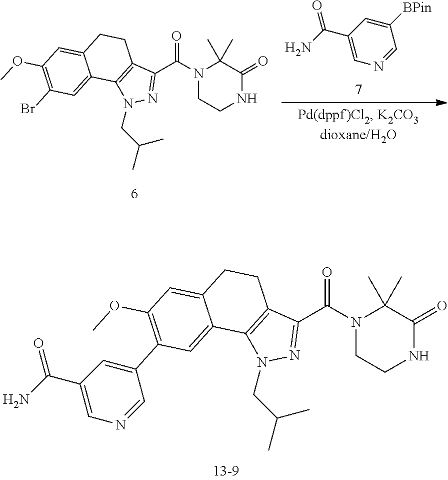

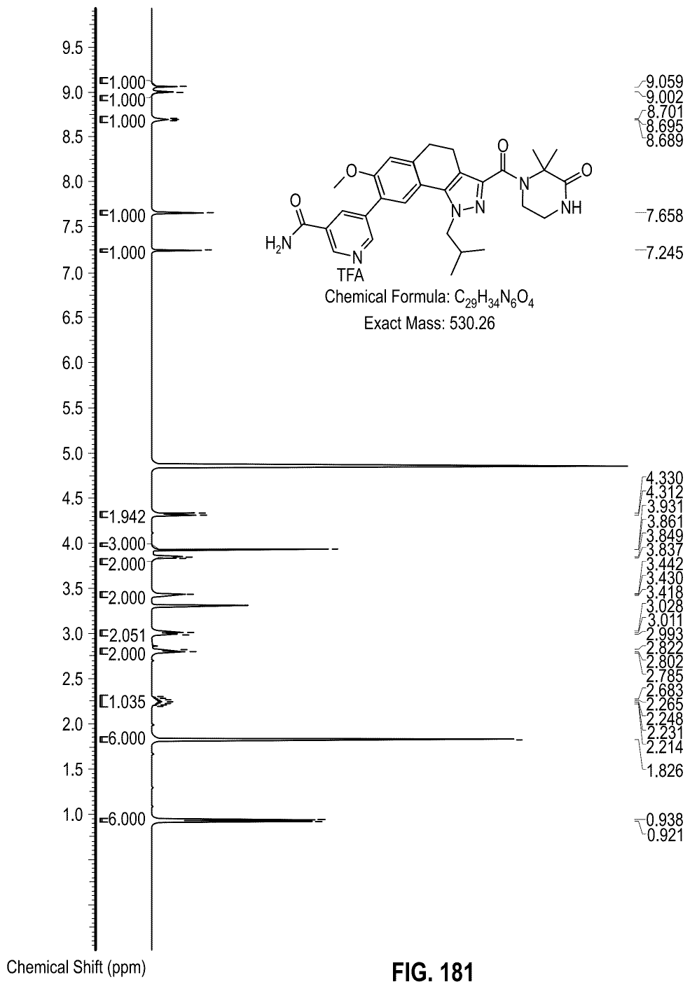

To a mixture of 4-(8-bromo-1-isobutyl-7-methoxy-4,5-dihydrobenzo[g]indazole-3-carbonyl)-3,3-dimethyl-piperazin-2-one (50 mg, 102.17 μmol, 1 eq), 5-(4,4,5,5-tetramethyl-1,3,2-dioxaborolan-2-yl)pyridine-3-carboxamide (38.02 mg, 153.25 μmol, 1.5 eq) and K2CO3 (42.36 mg, 306.50 μmol, 3 eq) in dioxane (1.5 mL) and H2O (0.4 mL) was added Pd(dppf)Cl2 (7.48 mg, 10.22 μmol, 0.1 eq) under N2 atmosphere. The mixture was stirred at 80° C. for 16 hr under N2 atmosphere. The reaction mixture was diluted with H2O (15 mL) and extracted with EtOAc (15 mL*3). The combined organic layers were washed with brine (20 mL*2), dried over anhydrous Na2SO4, filtered and concentrated under reduced pressure to give a residue. The residue was purified by prep-HPLC (column: Phenomenex Luna C18 150*25 mm*10 um; mobile phase:[water (TFA)-ACN]; gradient: 20%-50% B over 9 min). Compound 5-[3-(2,2-dimethyl-3-oxo-piperazine-1-carbonyl)-1-isobutyl-7-methoxy-4,5-dihydrobenzo[g]indazol-8-yl]pyridine-3-carboxamide (13.22 mg, 20.51 μmol, 20.07% yield, 100% purity, TFA) was obtained as a yellow solid. LCMS (ESI): m/z[M+H] calcd for C29H35N6O4: 531.26; found: 531.3. 1H NMR: (400 MHz, METHANOL-d4) δ=9.06 (s, 1H), 9.00 (br s, 1H), 8.73-8.66 (m, 1H), 7.66 (s, 1H), 7.24 (s, 1H), 4.32 (d, J=7.5 Hz, 2H), 3.93 (s, 3H), 3.88-3.81 (m, 2H), 3.46-3.40 (m, 2H), 3.04-2.98 (m, 2H), 2.84-2.77 (m, 2H), 2.25 (quind, J=6.8, 13.7 Hz, 1H), 1.83 (s, 6H), 0.93 (d, J=6.6 Hz, 6H). FIG. 181 shows the nuclear magnetic resonance of Compound 13-9.

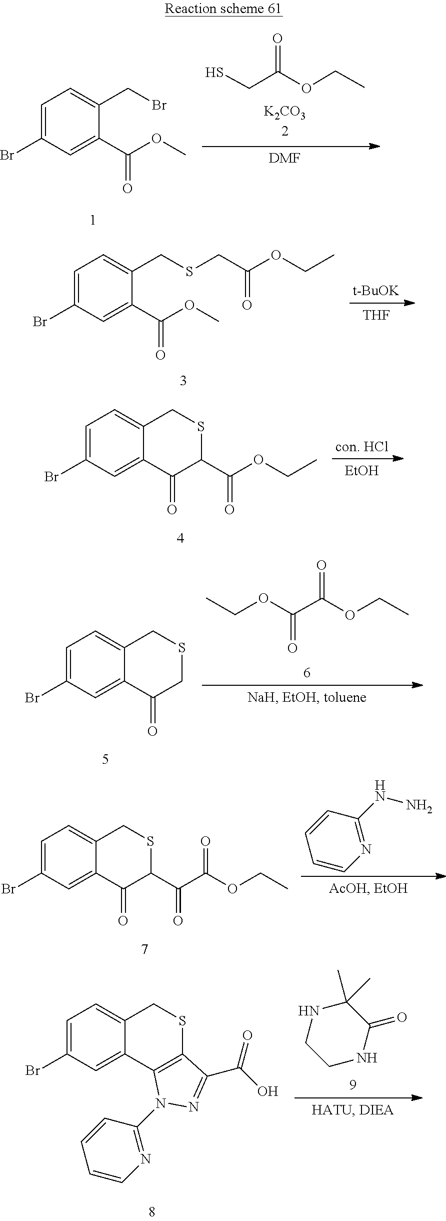

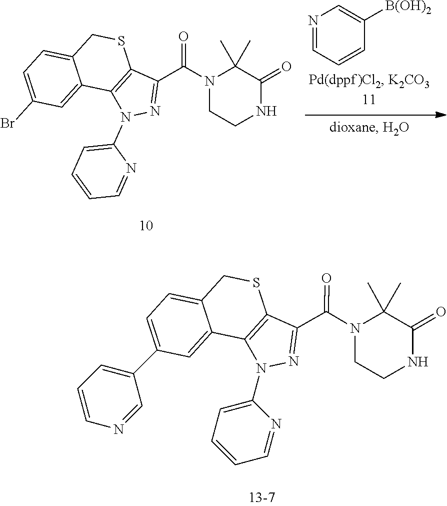

### Synthesis of methyl 5-bromo-2-[(2-ethoxy-2-oxo-ethyl)sulfanylmethyl]benzoate

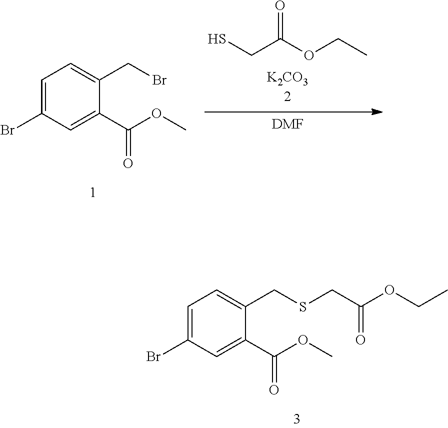

To a solution of methyl 5-bromo-2-(bromomethyl)benzoate (5.90 g, 19.15 mmol, 1.05 eq) and ethyl 2-sulfanylacetate (2.19 g, 18.24 mmol, 2 mL, 1 eq) in DMF (50 mL) was added K2CO3 (5.04 g, 36.48 mmol, 2 eq) at 0° C., the mixture was stirred at 0˜30° C. for 3 hours. The reaction was quenched with ice water(100 mL), extracted with EtOAc (30 mL*2), the organic layers were combined and washed with water(50 mL), brine(50 mL), dried over anhydrous Na2SO4, filtered and concentrated to afford methyl 5-bromo-2-[(2-ethoxy-2-oxo-ethyl)sulfanylmethyl]benzoate (6 g, crude) as yellow oil.

### Synthesis of ethyl 6-bromo-4-oxo-isothiochromane-3-carboxylate

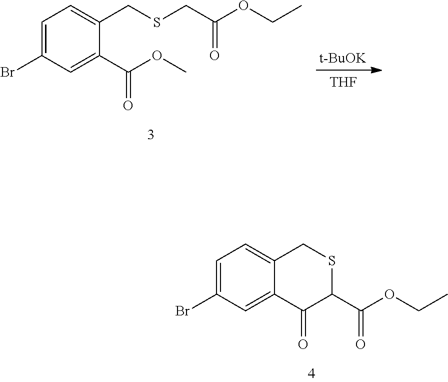 [SMILES: "t-BuOK" ; CCOC(=O)CSCc1ccc(Br)cc1C(=O)OC ; CCOC(=O)C1SCc2ccc(Br)cc2C1=O ; "THF"]

To a solution of methyl 5-bromo-2-[(2-ethoxy-2-oxo-ethyl)sulfanylmethyl]benzoate (5 g, 14.40 mmol, 1 eq) in THF (40 mL) was added t-BuOK (1 M, 14.40 mL, 1 eq) under 0° C., the mixture was stirred at 0˜20° C. for 10 min. The reaction was quenched with sat. aq. NH4Cl (30 mL), extracted with EtOAc (30*2 mL), the organic layers were combined and washed with brine (30 mL), dried over anhydrous Na2SO4, filtered and concentrated to get a residue. The residue was purified by column chromatography (SiO2, Petroleum ether/Ethyl acetate=50/1 to 10/1) to afford ethyl 6-bromo-4-oxo-isothiochromane-3-carboxylate (4.2 g, 13.33 mmol, 92.54% yield) as yellow solid.

### Synthesis of 6-bromoisothiochroman-4-one

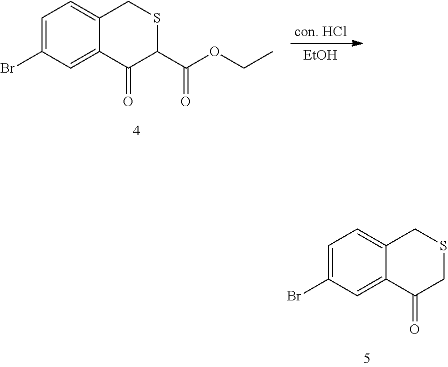

To a solution of ethyl 6-bromo-4-oxo-isothiochromane-3-carboxylate (3.5 g, 11.10 mmol, 1 eq) in EtOH (20 mL) was added HCl (12 M, 21.88 mL, 23.64 eq), the mixture was stirred at 110° C. for 3 hours. The reaction was cooled to room temperature, diluted with water (50 mL), extracted with EtOAc (50 mL*2), the organic layers were combined and dried over anhydrous Na2SO4, filtered and concentrated to get a residue. The residue was purified by column chromatography (SiO2, Petroleum ether/Ethyl acetate=50/1 to 10/1) to afford 6-bromoisothiochroman-4-one (1.2 g, 4.94 mmol, 44.45% yield) as yellow solid. 1H NMR (400 MHz, CHLOROFORM-d) δ=8.22 (d, J=2.1 Hz, 1H), 7.58 (dd, J=2.0, 8.0 Hz, 1H), 7.11 (d, J=8.0 Hz, 1H), 3.88 (s, 2H), 3.57-3.54 (m, 2H).

### Synthesis of ethyl 2-(6-bromo-4-oxoisothiochroman-3-yl)-2-oxoacetate

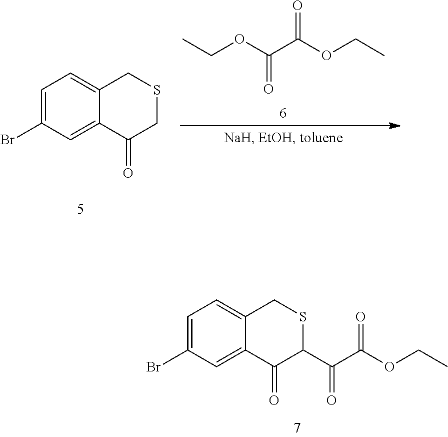

To a solution of NaH (394.83 mg, 9.87 mmol, 60% purity, 3 eq) in toluene (10 mL) was added 6-bromoisothiochroman-4-one (800 mg, 3.29 mmol, 1 eq) and diethyl oxalate (1.44 g, 9.87 mmol, 1.35 mL, 3 eq) at 0° C. under N2. And then EtOH (151.59 mg, 3.29 mmol, 1 eq) was added dropwise at 0° C. The mixture was stirred at 25° C. for 4 hr. The reaction mixture was diluted with H2O 50 mL and extracted with EtOAc 100 mL (50 mL*2). The combined organic layers were washed with brine 100 mL (50 mL*2), dried over Na2SO4, filtered and concentrated under reduced pressure to give a residue. The crude product ethyl 2-(6-bromo-4-oxoisothiochroman-3-yl)-2-oxoacetate (850 mg, 2.48 mmol, 75.27% yield) as a brown oil was used into the next step without further purification.

### Synthesis of 8-bromo-1-(2-pyridyl)-5H-isothiochromeno[4,3-c]pyrazole-3-carboxylic acid

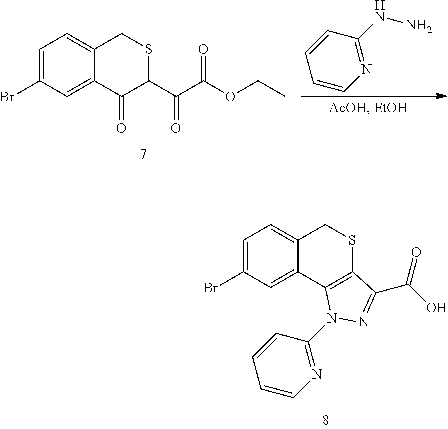

To a solution of ethyl 2-(6-bromo-4-oxo-isothiochroman-3-yl)-2-oxo-acetate (0.2 g, 582.76 μmol, 1 eq) in EtOH (3 mL) was added 2-pyridylhydrazine (95.39 mg, 874.14 μmol, 1.5 eq), then AcOH (1.57 g, 26.20 mmol, 1.5 mL, 44.96 eq) was added, the mixture was stirred at 80° C. for 1 hour. The reaction was quenched with water (10 mL), extracted with EtOAc (10 mL), the organic layer was separated and dried over anhydrous Na2SO4, filtered and concentrated to get a residue. The residue was purified by prep-TLC(Petroleum ether/Ethyl acetate=3:1) to afford 8-bromo-1-(2-pyridyl)-5H-isothiochromeno[4,3-c]pyrazole-3-carboxylic acid (40 mg, 103.03 μmol, 17.68% yield) as a yellow solid.

### Synthesis of 4-[8-bromo-1-(2-pyridyl)-5H-isothiochromeno[4,3-c]pyrazole-3-carbonyl]-3,3-dimethyl-piperazin-2-one

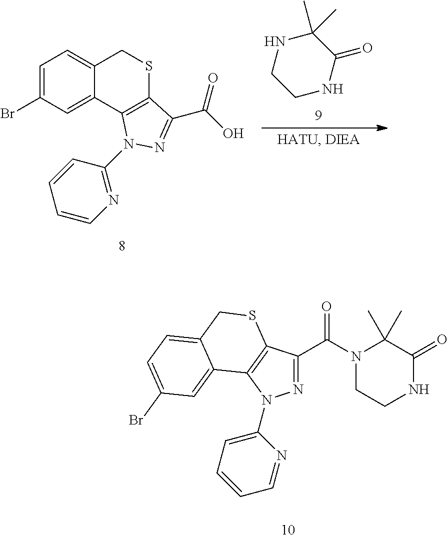

To a solution of 8-bromo-1-(2-pyridyl)-5H-isothiochromeno[4,3-c]pyrazole-3-carboxylic acid (15 mg, 38.64 μmol, 1 eq) in DMF (2 mL) was added HATU (17.63 mg, 46.36 μmol, 1.2 eq), then DIEA (14.98 mg, 115.91 μmol, 20.19 μL, 3 eq) was added,then 3,3-dimethylpiperazin-2-one (6.00 mg, 46.81 μmol, 1.21 eq) was added, the mixture was stirred at 20° C. for 13 hours. The reaction was diluted with water(5 mL), extracted with EtOAc (5 mL*2), the organic layers were combined and washed with brine(5 mL), dried over anhydrous Na2SO4, filtered and concentrated to get a residue. The residue was purified by prep-TLC (Petroleum ether/Ethyl acetate=1:2) to afford 4-[8-bromo-1-(2-pyridyl)-5H-isothiochromeno[4,3-c]pyrazole-3-carbonyl]-3,3-dimethyl-piperazin-2-one (5 mg, 10.03 μmol, 25.97% yield) as white solid.

### Synthesis of Compound 13-7

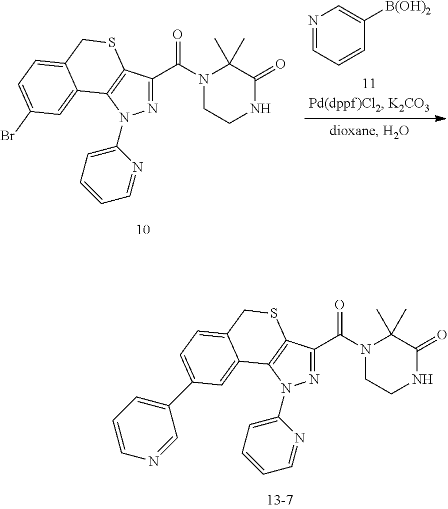

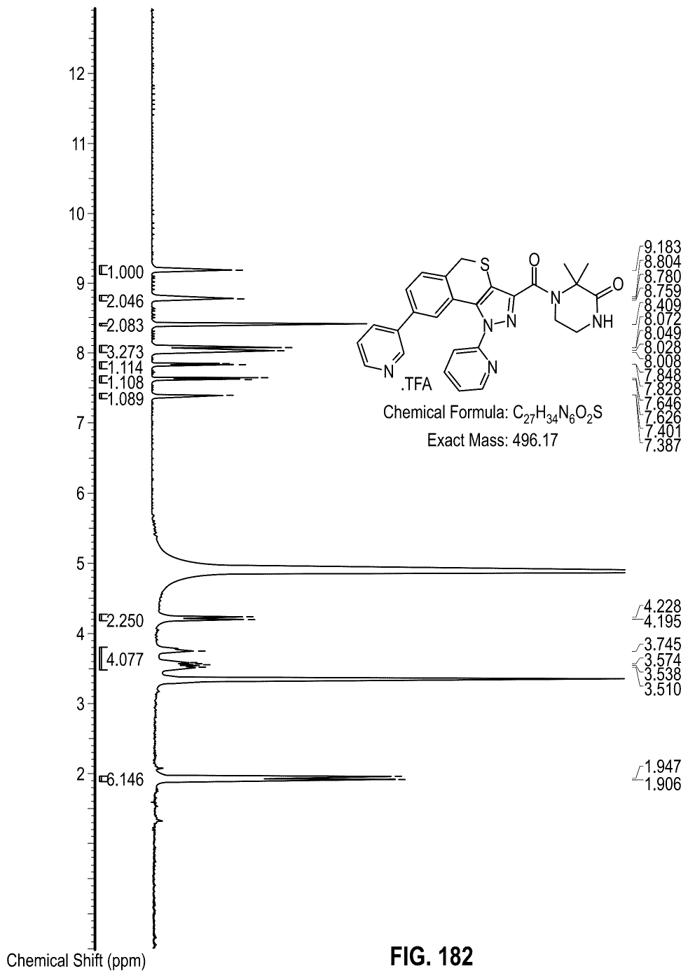

To a solution of 4-[8-bromo-1-(2-pyridyl)-5H-isothiochromeno[4,3-c]pyrazole-3-carbonyl]-3,3-dimethyl-piperazin-2-one (5 mg, 10.03 μmol, 1 eq) and 3-pyridylboronic acid (2.47 mg, 20.06 μmol, 2 eq) in dioxane (1 mL) and H2O (0.2 mL) was added Pd(dppf)Cl2 (1.47 mg, 2.01 μmol, 0.2 eq) and K2CO3 (4.16 mg, 30.10 μmol, 3 eq).The mixture was stirred at 60° C. for 2 h. The reaction was cooled to room temperature. and the dioxane layer was separated and purified by Pre-HPLC (column: Phenomenex Luna C18 150*25 mm*10 um; mobile phase:[water (TFA)-ACN]; gradient: 15%-45% B over 9 min) to afford 3,3-dimethyl-4-[1-(2-pyridyl)-8-(3-pyridyl)-5H-isothiochromeno[4,3-c]pyrazole-3-carbonyl]piperazin-2-one (2.35 mg, 4.73 μmol, 47.17% yield) as white solid. LCMS (ESI): m/z[M+H] calcd for C27H25N6O2S: 497.2; found: 497.2. 1H NMR (400 MHz, METHANOL-d4) δ=9.18 (br s, 1H), 8.82-8.75 (m, 2H), 8.41 (br s, 2H), 8.10-8.00 (m, 3H), 7.84 (br d, J=8.1 Hz, 1H), 7.64 (d, J=7.8 Hz, 1H), 7.45-7.28 (m, 1H), 4.21 (br d, J=13.2 Hz, 3H), 3.74 (br s, 1H), 3.61-3.47 (m, 3H), 1.93 (br d, J=16.5 Hz, 7H). FIG. 182 shows the nuclear magnetic resonance of Compound 13-7.

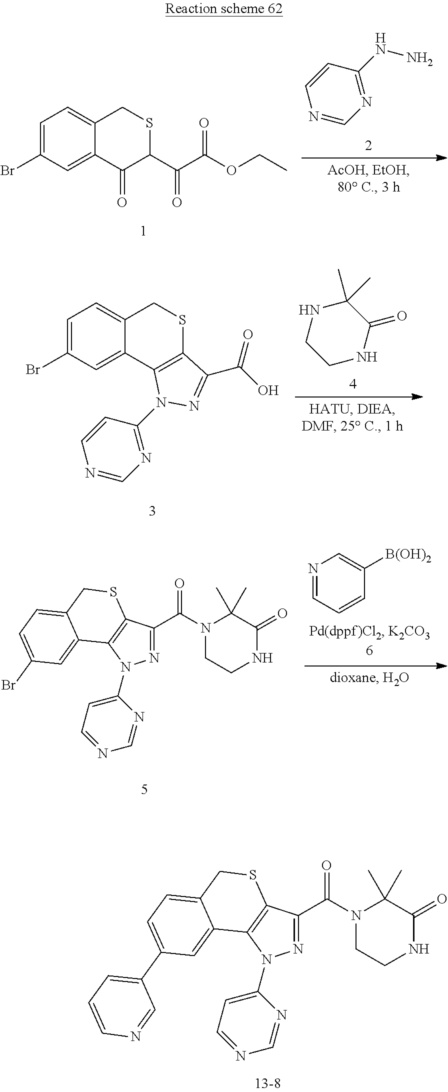

### Synthesis of 8-bromo-1-pyrimidin-4-yl-5H-isothiochromeno[4,3-c]pyrazole-3-carboxylic acid

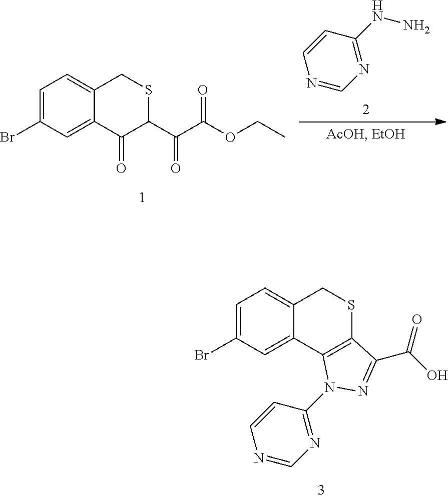 [SMILES: CCOC(=O)C(=O)C1SCc2ccc(Br)cc2C1=O ; NNc1ccncn1 ; O=C(O)c1nn(-c2ccncn2)c2c1SCc1ccc(Br)cc1-2]

To a solution of ethyl 2-(6-bromo-4-oxo-isothiochroman-3-yl)-2-oxo-acetate (50 mg, 145.69 μmol, 1 eq) in EtOH (1 mL) was added pyrimidin-4-ylhydrazine (17.65 mg, 160.26 μmol, 1.1 eq), then AcOH (524.50 mg, 8.73 mmol, 0.5 mL, 59.95 eq) was added, the mixture was stirred at 80° C. for 2 hours. The reaction was cooled to room temperature. The solid was collected by filtration to afford 8-bromo-1-pyrimidin-4-yl-5H-isothiochromeno[4,3-c]pyrazole-3-carboxylic acid (20 mg, 51.38 μmol, 35.27% yield) as yellow solid.

### Synthesis of 4-(8-bromo-1-pyrimidin-4-yl-5H-isothiochromeno[4,3-c]pyrazole-3-carbonyl)-3,3-dimethyl-piperazin-2-one

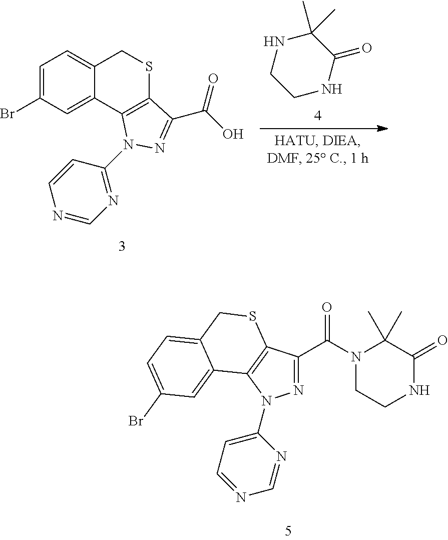

To a solution of 8-bromo-1-pyrimidin-4-yl-5H-isothiochromeno[4,3-c]pyrazole-3-carboxylic acid (20 mg, 51.38 μmol, 1 eq) in DMF (2 mL) was added HATU (23.45 mg, 61.66 μmol, 1.2 eq), then DIEA (19.92 mg, 154.15 μmol, 26.85 μL, 3 eq) was added, then 3,3-dimethylpiperazin-2-one (7.24 mg, 56.52 μmol, 1.1 eq) was added, the mixture was stirred at 25° C. for 1 hour. The reaction was quenched with water (5 mL), extracted with EtOAc (5 mL*2), the organic layer was separated and dried over anhydrous Na2SO4, filtered and concentrated to get a residue. The residue was purified by Pre-TLC (Petroleum ether/Ethyl acetate=1:2) to afford 4-(8-bromo-1-pyrimidin-4-yl-5H-isothiochromeno[4,3-c]pyrazole-3-carbonyl)-3,3-dimethyl-piperazin-2-one (5 mg, 10.01 μmol, 19.49% yield) as a white solid.

### Synthesis of Compound 13-8

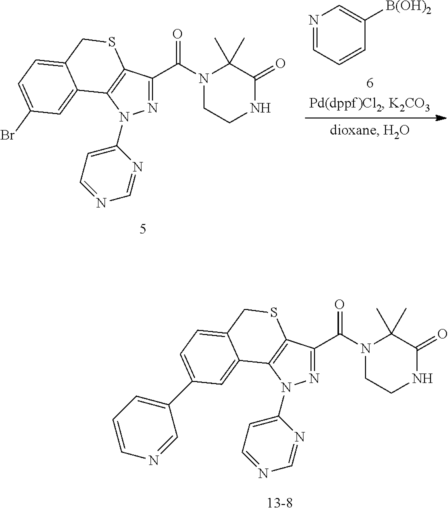

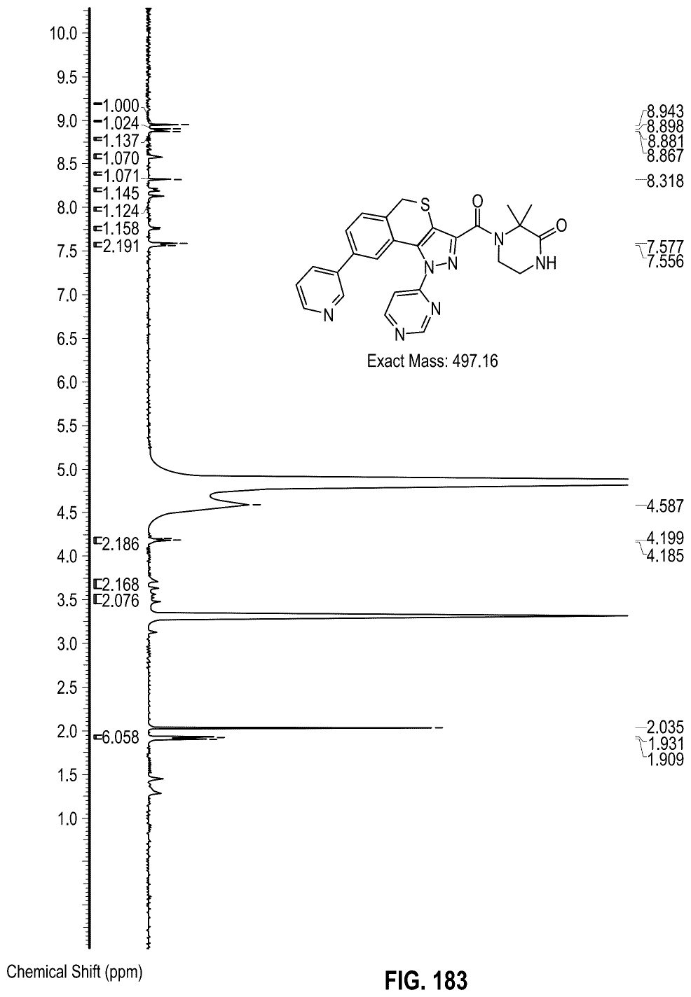

To a solution of 4-(8-bromo-1-pyrimidin-4-yl-5H-isothiochromeno[4,3-c]pyrazole-3-carbonyl)-3,3-dimethyl-piperazin-2-one (5 mg, 10.01 μmol, 1 eq) and 3-pyridylboronic acid (2.46 mg, 20.02 μmol, 2 eq) in dioxane (1.5 mL) and H2O (0.5 mL) was added Pd(dppf)Cl2 (1.47 mg, 2.00 μmol, 0.2 eq) and K2CO3 (4.15 mg, 30.04 μmol, 3 eq), the mixture was stirred at 80° C. for 12 hours. The dioxane layer was separated and purified by Pre-HPLC with the following method: column: Waters Xbridge 150*25 mm*5 um; mobile phase:[water (NH4HCO3)-ACN]; gradient: 20%-50% B over 9 min to afford 3,3-dimethyl-4-[8-(3-pyridyl)-1-pyrimidin-4-yl-5H-isothiochromeno[4,3-c]pyrazole-3-carbonyl]piperazin-2-one (0.73 mg, 1.43 μmol, 14.31% yield, 97.67% purity) as white solid. LCMS (ESI): m/z[M+H] calcd for C26H23N7O2S: 498.2; found: 498.1. 1H NMR (400 MHz, METHANOL-d4) δ=8.95 (s, 1H), 8.89-8.91 (m, 1H), 8.89 (d, J=5.6 Hz, 1H), 8.62-8.58 (m, 1H), 8.34 (s, 1H), 8.24-8.19 (m, 1H), 8.16-8.14 (m, 1H), 7.78-7.78 (m, 1H), 7.80-7.76 (m, 1H), 7.60-7.56 (m, 2H), 4.21 (d, J=5.6 Hz, 2H), 3.77-3.66 (m, 2H), 3.62-3.49 (m, 2H), 1.92 (d, J=8.8 Hz, 6H). FIG. 183 shows the nuclear magnetic resonance of Compound 13-8.

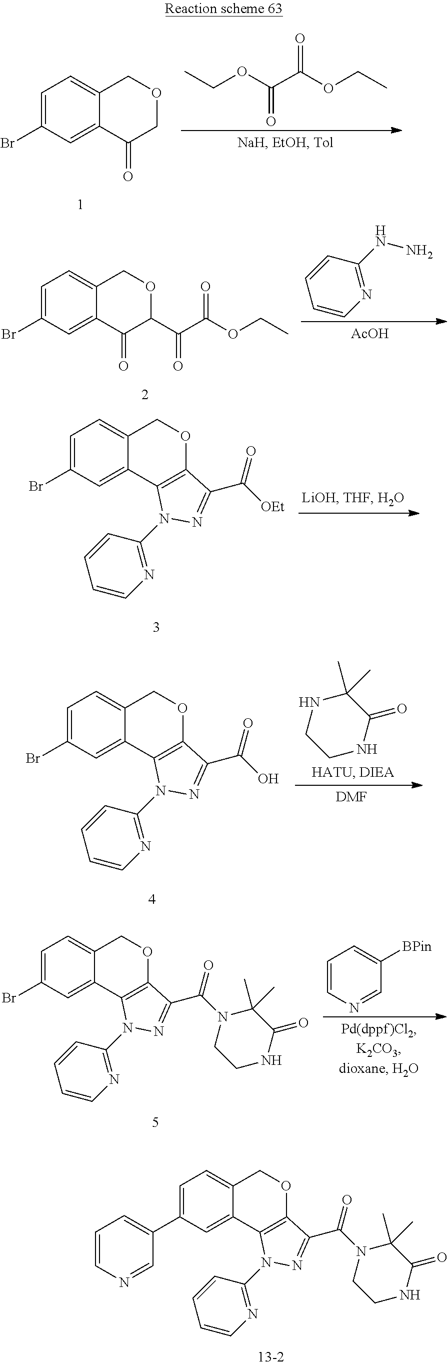

### Synthesis of ethyl 2-(6-bromo-4-oxoisochroman-3-yl)-2-oxoacetate

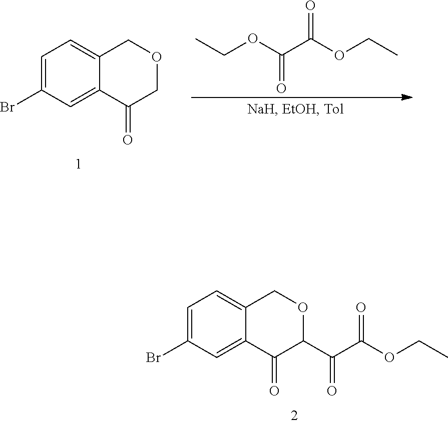

To a solution of NaH (1.06 g, 26.43 mmol, 60% purity, 3 eq) in toluene (10 mL) was added 6-bromoisochroman-4-one (2 g, 8.81 mmol, 1 eq) and diethyl oxalate (3.86 g, 26.43 mmol, 3.61 mL, 3 eq) in toluene (20 mL) at 0° C. under N2. After addition of EtOH (405.79 mg, 8.81 mmol, 1 eq) dropwise at 0° C., the mixture was stirred at 25° C. for 4 hr. The reaction mixture was quenched by addition of 1M HCl (20 mL), extracted with EtOAc (20 mL), the organic phase was washed with brine, dried with anhydrous Na2SO4, filtered and filtrate was concentrated in vacuo. The crude product was triturated with MTBE at 25° C. for 30 min. Compound ethyl 2-(6-bromo-4-oxoisochroman-3-yl)-2-oxoacetate (1.65 g, 5.04 mmol, 57.26% yield) was obtained as a yellow solid. 1H NMR (400 MHz, CHLOROFORM-d) δ=12.75-12.33 (m, 1H), 8.09 (d, J=2.0 Hz, 1H), 7.69 (dd, J=2.0, 8.1 Hz, 1H), 7.11 (d, J=8.2 Hz, 1H), 5.06 (s, 2H), 4.40 (q, J=7.1 Hz, 2H), 1.41 (t, J=7.2 Hz, 3H).

### Synthesis of ethyl 8-bromo-1-(pyridin-2-yl)-1,5-dihydroisochromeno[4,3-c]pyrazole-3-carboxylate

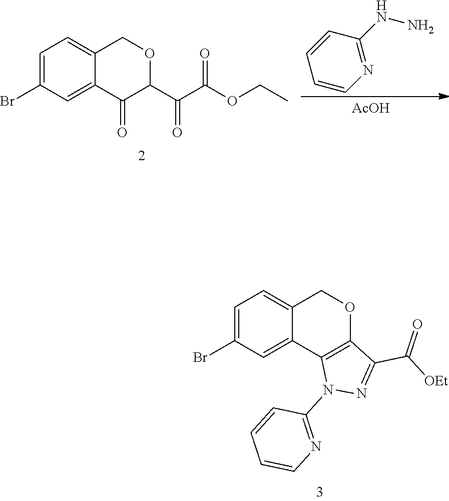

A mixture of ethyl 2-(6-bromo-4-oxoisochroman-3-yl)-2-oxoacetate (300 mg, 917.08 μmol, 1 eq) and 2-hydrazineylpyridine (100.08 mg, 917.08 μmol, 1 eq) in AcOH (314.70 mg, 5.24 mmol, 300.00 μL, 5.71 eq) The mixture was stirred at 120° C. for 2 hr. The reaction mixture was concentrated under reduced pressure to give a residue. The residue was purified by reverse phase column (FA condition). Compound ethyl 8-bromo-1-(pyridin-2-yl)-1,5-dihydroisochromeno[4,3-c]pyrazole-3-carboxylate (17 mg, 41.63 μmol, 4.54% yield, 98% purity) was obtained as a yellow solid. LCMS (ESI): m/z[M+H] calcd for C18H15BrO3N3: 400.12; found: 399.9, 401.9. 1H NMR (400 MHz, DMSO-d6) δ=8.57 (dd, J=1.1, 4.7 Hz, 1H), 8.18 (dt, J=1.8, 7.8 Hz, 1H), 7.93-7.89 (m, 1H), 7.65-7.61 (m, 1H), 7.55 (dd, J=1.9, 8.1 Hz, 1H), 7.38 (d, J=8.1 Hz, 1H), 7.20 (d, J=1.9 Hz, 1H), 5.31 (s, 2H), 4.39-4.30 (m, 2H), 1.33-1.29 (m, 3H).

### Synthesis of 8-bromo-1-(pyridin-2-yl)-1,5-dihydroisochromeno[4,3-c]pyrazole-3-carboxylic acid

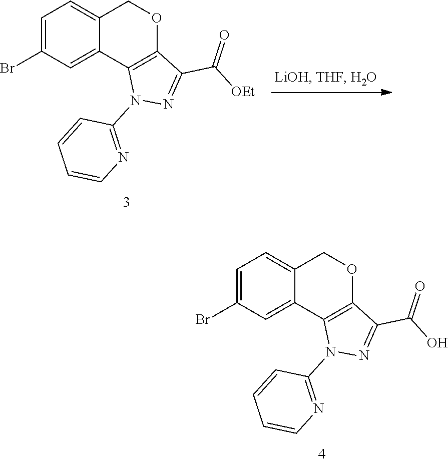 [SMILES: CCOC(=O)c1nn(-c2ccccn2)c2c1OCc1ccc(Br)cc1-2 ; O=C(O)c1nn(-c2ccccn2)c2c1OCc1ccc(Br)cc1-2]

To a solution of ethyl 8-bromo-1-(2-pyridyl)-5H-isochromeno[4,3-c]pyrazole-3-carboxylate (17 mg, 41.63 μmol, 1 eq) in H2O (0.5 mL) and EtOH (0.5 mL) was added LiOH·H2O (6.99 mg, 166.51 μmol, 4 eq). The mixture was stirred at 25° C. for 2 hr. The reaction mixture was diluted with H2O 20 mL and extracted with EtOAc 40 mL (20 mL*2). The aqueous layer was acidified to pH=5 with 1 M HCl aqueous solution, and then extracted with EtOAc 40 mL (20 mL*2). The combined organic layers were washed with brine 40 mL (20 mL*2), dried over Na2SO4, filtered and concentrated under reduced pressure to give a residue. Compound 8-bromo-1-(pyridin-2-yl)-1,5-dihydroisochromeno[4,3-c]pyrazole-3-carboxylic acid (15 mg, 40.30 μmol, 96.82% yield) was obtained as a yellow solid. 1H NMR (400 MHz, DMSO-d6) δ=8.56 (d, J=4.3 Hz, 1H), 8.20-8.14 (m, 1H), 7.91 (d, J=8.1 Hz, 1H), 7.61 (dd, J=5.1, 7.0 Hz, 1H), 7.54 (dd, J=2.0, 7.9 Hz, 1H), 7.38 (d, J=8.2 Hz, 1H), 7.23 (d, J=1.8 Hz, 1H), 5.28 (s, 2H).

### Synthesis of 4-(8-bromo-1-(pyridin-2-yl)-1,5-dihydroisochromeno[4,3-c]pyrazole-3-carbonyl)-3,3-dimethylpiperazin-2-one

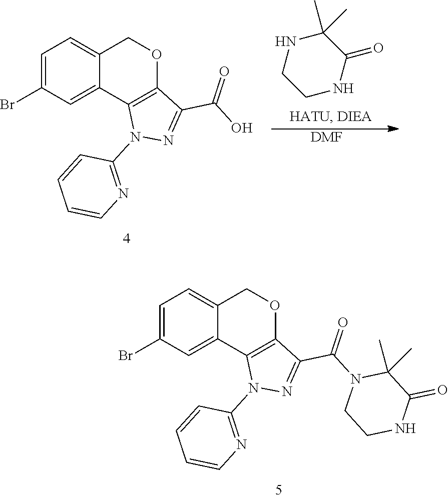

To a solution of 8-bromo-1-(2-pyridyl)-5H-isochromeno[4,3-c]pyrazole-3-carboxylic acid (15 mg, 40.30 μmol, 1 eq) in DMF (1 mL) was added dropwise HATU (22.99 mg, 60.46 μmol, 1.5 eq) and DIEA (15.63 mg, 120.91 μmol, 21.06 μL, 3 eq) at 25° C. After addition, the mixture was stirred at this temperature for 30 min, and then 3,3-dimethylpiperazin-2-one (7.75 mg, 60.46 μmol, 1.5 eq) was added dropwise. The resulting mixture was stirred at 25° C. for 16 hr. The reaction mixture was diluted with H2O 20 mL and extracted with EtOAc 40 mL (20 mL*2). The combined organic layers were washed with brine 40 mL (20 mL*2), dried over Na2SO4, filtered and concentrated under reduced pressure to give a residue. The residue was purified by prep-TLC (SiO2, Petroleum ether/Ethyl acetate=0/1). Compound 4-(8-bromo-1-(pyridin-2-yl)-1,5-dihydroisochromeno[4,3-c]pyrazole-3-carbonyl)-3,3-dimethylpiperazin-2-one (6 mg, 12.44 μmol, 30.86% yield) was obtained as a yellow solid. LCMS (ESI): m/z[M+H] calcd for C22H21BrO3N5: 482.07; found: 482.0, 484.0.

### Synthesis of Compound 13-2

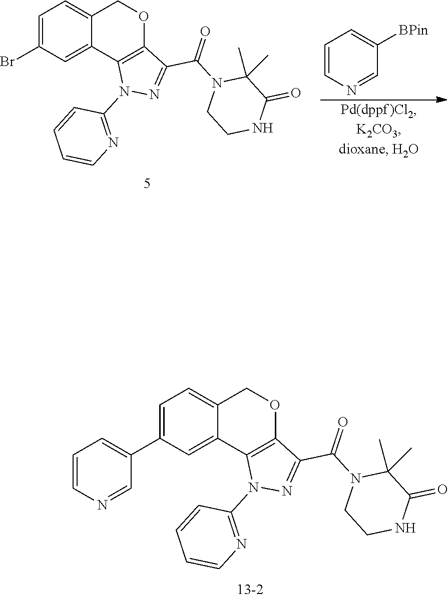

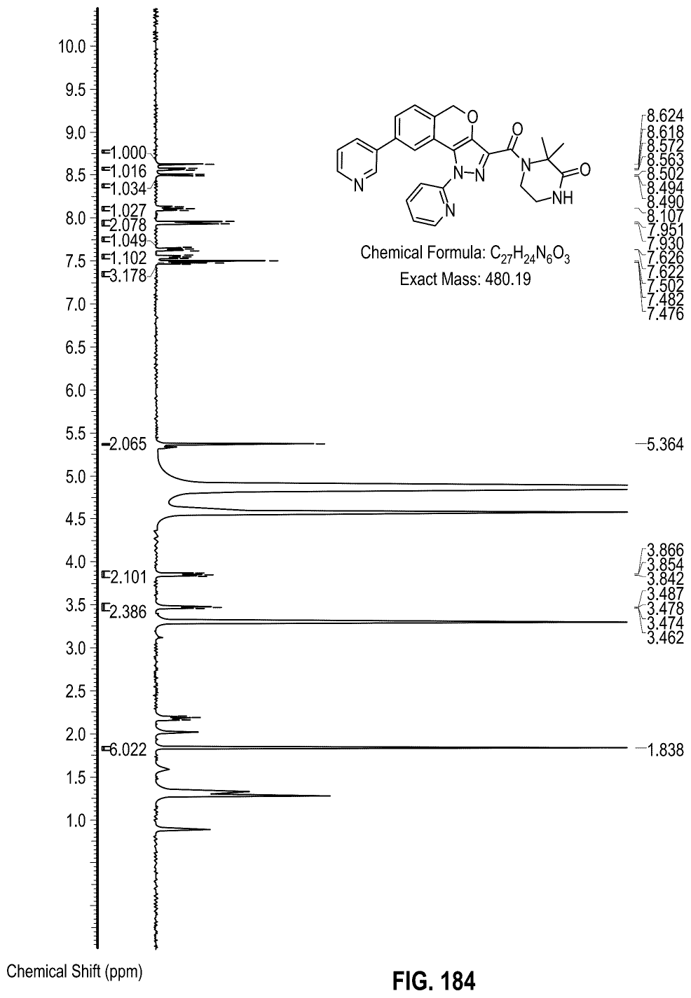

A mixture of 4-[8-bromo-1-(2-pyridyl)-5H-isochromeno[4,3-c]pyrazole-3-carbonyl]-3,3-dimethyl-piperazin-2-one (6 mg, 12.44 μmol, 1 eq), pyridin-3-ylboronic acid (3.06 mg, 24.88 μmol, 2 eq), Pd(dppf)Cl2 (910.21 pg, 1.24 μmol, 0.1 eq) and K2CO3 (5.16 mg, 37.32 μmol, 3 eq) in dioxane (1 mL) and H2O (0.5 mL), and then the mixture was stirred at 60° C. for 2 hr under N2 atmosphere. The reaction mixture was diluted with EtOAc (10 mL) and then filtered through a celite pad.The filtrate was concentrated. The residue was purified by twice prep-HPLC (column: Waters Xbridge 150*25 mm*5 um;mobile phase: [water (ammonia hydroxide v/v)-ACN];gradient:10%-40% B over 10 min), followed by lyophilization. Compound 3,3-dimethyl-4-(1-(pyridin-2-yl)-8-(pyridin-3-yl)-1,5-dihydroisochromeno[4,3-c]pyrazole-3-carbonyl)piperazin-2-one (0.76 mg, 1.57 μmol, 12.59% yield, 99% purity) was obtained as a white solid. LCMS (ESI): m/z[M+H] calcd for C27H25N6O3: 481.19; found: 481.2. 1H NMR (400 MHz, METHANOL-d4) δ=8.63-8.60 (m, 1H), 8.57 (dd, J=1.1, 5.0 Hz, 1H), 8.50 (dd, J=1.5, 4.9 Hz, 1H), 8.14-8.08 (m, 1H), 7.96-7.91 (m, 2H), 7.63 (dd, J=1.6, 7.8 Hz, 1H), 7.55 (ddd, J=0.9, 5.5, 6.8 Hz, 1H), 7.51-7.45 (m, 3H), 5.36 (s, 2H), 3.89-3.82 (m, 2H), 3.48 (dd, J=4.1, 5.6 Hz, 2H), 1.84 (s, 6H). FIG. 184 shows the nuclear magnetic resonance of Compound 13-2.

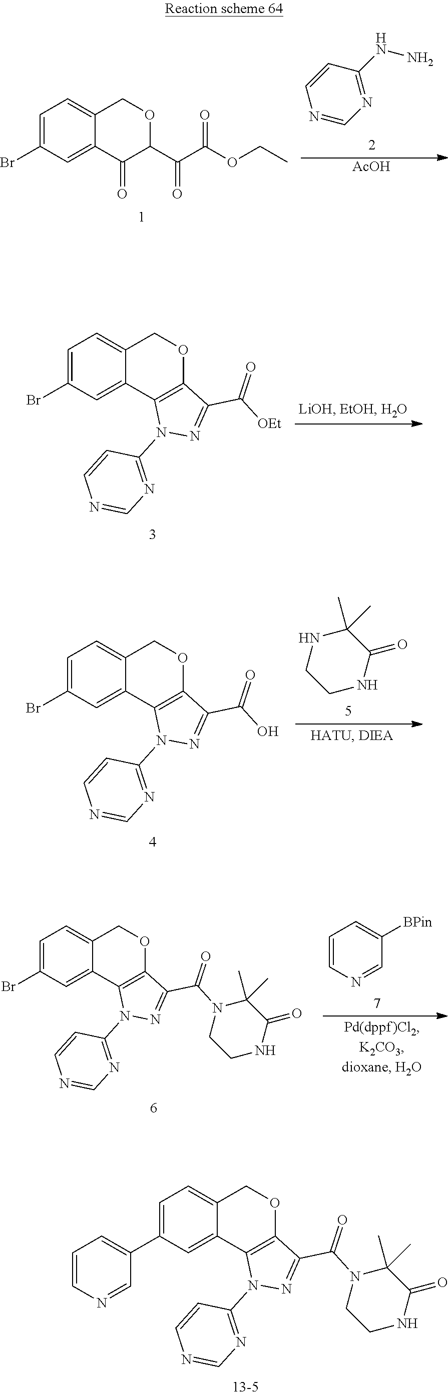

### Synthesis of Compound 13-5

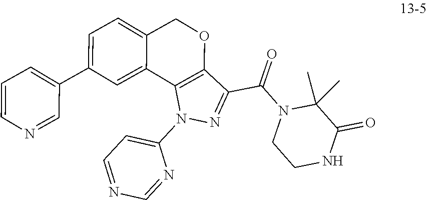 [SMILES: CC1(C)C(=O)NCCN1C(=O)c1nn(-c2ccncn2)c2c1OCc1ccc(-c3cccnc3)cc1-2]

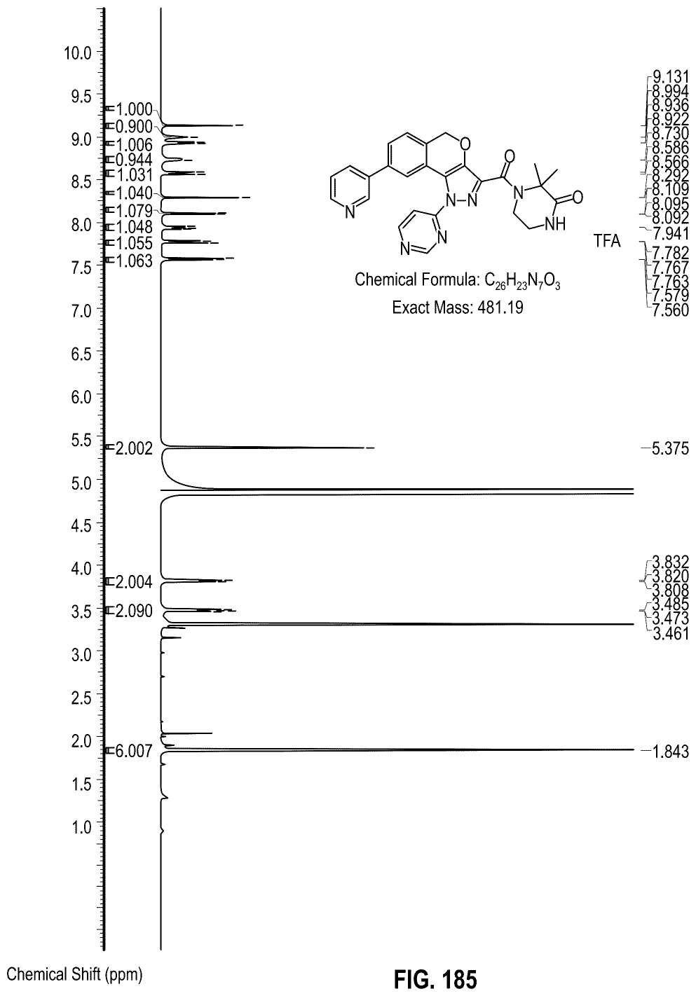

Compound 13-5 was synthesized by a procedure similar to Reaction scheme 64. LCMS (ESI): m/z[M+H] calcd for C26H24N7O3: 482.19; found: 482.1. 1H NMR (400 MHz, METHANOL-d4) δ=9.13 (s, 1H), 8.99 (br s, 1H), 8.93 (br d, J=5.5 Hz, 1H), 8.73 (br s, 1H), 8.58 (br d, J=8.3 Hz, 1H), 8.29 (d, J=1.6 Hz, 1H), 8.10 (dd, J=1.0, 5.8 Hz, 1H), 7.94 (dd, J=5.5, 8.0 Hz, 1H), 7.77 (dd, J=1.8, 7.8 Hz, 1H), 7.57 (d, J=7.9 Hz, 1H), 5.37 (s, 2H), 3.86-3.77 (m, 2H), 3.51-3.44 (m, 2H), 1.84 (s, 6H). FIG. 185 shows the nuclear magnetic resonance of Compound 13-5.

### Example 14

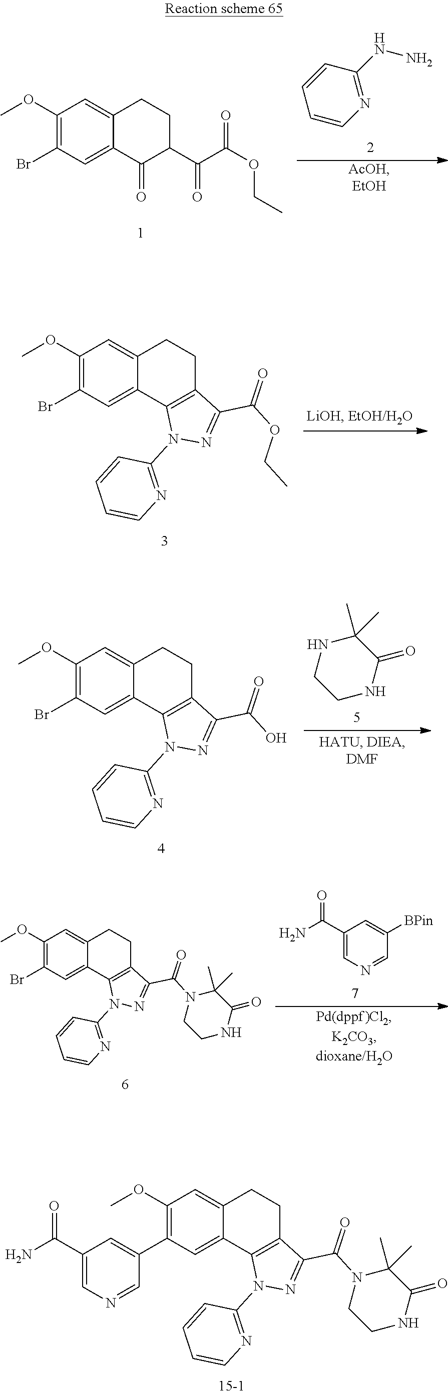

### Synthesis of ethyl 8-bromo-7-methoxy-1-(2-pyridyl)-4,5-dihydrobenzo[g]indazole-3-carboxylate

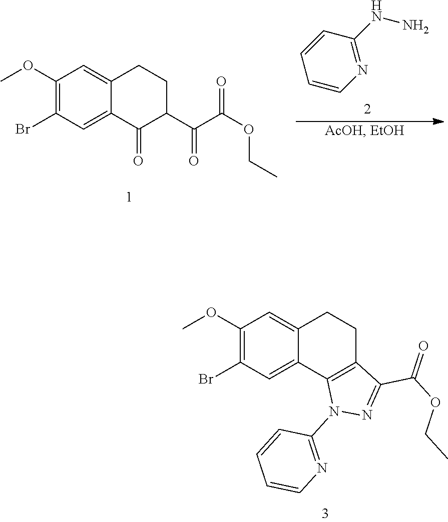

A mixture of ethyl 2-(7-bromo-6-methoxy-1-oxo-tetralin-2-yl)-2-oxo-acetate (0.5 g, 1.41 mmol, 1 eq), 2-pyridylhydrazine (168.99 mg, 1.55 mmol, 1.1 eq) and AcOH (845.37 mg, 14.08 mmol, 805.89 μL, 10 eq) in EtOH (8 mL) was stirred at 80° C. for 16 hr under N₂ atmosphere. The reaction mixture was concentrated. The residue was purified by flash silica gel chromatography (ISCO®; 10 g SepaFlash® Silica Flash Column, Eluent of 0˜30% Ethyl acetate/Petroleum ethergradient A 40 mL/min). Compound ethyl 8-bromo-7-methoxy-1-(2-pyridyl)-4,5-dihydrobenzo[g]indazole-3-carboxylate (470 mg, 1.10 mmol, 77.9600 yield) was obtained as a yellow solid. 1H NMR (400 MHz, CHLOROFORM-d) δ=8.73 (dd, J=1.1, 4.9 Hz, 1H), 8.18-8.11 (m, 1H), 8.02 (d, J=7.9 Hz, 1H), 7.64-7.59 (m, 1H), 7.45 (s, 1H), 7.05-7.02 (m, 1H), 4.64 (q, J=7.1 Hz, 2H), 4.10 (s, 3H), 3.28-3.22 (m, 2H), 3.19-3.14 (m, 2H), 1.62 (t, J=7.1 Hz, 3H).

### Synthesis of 8-bromo-7-methoxy-1-(2-pyridyl)-4,5-dihydrobenzo[g]indazole-3-carboxylic acid

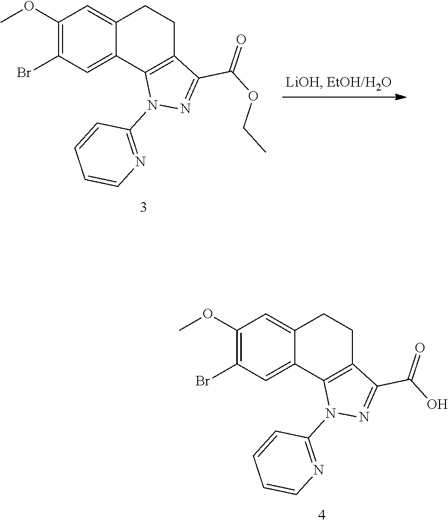

A mixture of ethyl 8-bromo-7-methoxy-1-(2-pyridyl)-4,5-dihydrobenzo[g]indazole-3-carboxylate (0.45 g, 1.05 mmol, 1 eq) and LiOH·H2O (176.37 mg, 4.20 mmol, 3 mL, 4 eq) in EtOH (6 mL) was stirred at 25° C. for 1 hr. The reaction mixture was poured into ice-water (20 mL) and neutralized with TN HCl. The aqueous phase was extracted with ethyl acetate (20 mL*3). The combined organic phase was washed with brine (30 mL), dried with anhydrous Na2SO4, filtered and concentrated in vacuum. The crude product 8-bromo-7-methoxy-1-(2-pyridyl)-4, 5-dihydrobenzo[g]indazole-3-carboxylic acid (0.24 g, crude) as a yellow solid was used into the next step without further purification. 1H NMR (400 MHz, METHANOL-d4) δ=8.55 (d, J=4.8 Hz, 1H), 8.20-8.13 (m, 1H), 7.81 (d, J=7.9 Hz, 1H), 7.64 (dd, J=5.0, 7.4 Hz, 1H), 7.05 (s, 1H), 6.82 (s, 1H), 3.89 (s, 3H), 3.04-2.98 (m, 4H).

### Synthesis of 4-[8-bromo-7-methoxy-1-(2-pyridyl)-4,5-dihydrobenzo[g]indazole-3-carbonyl]-3,3-dimethyl-piperazin-2-one

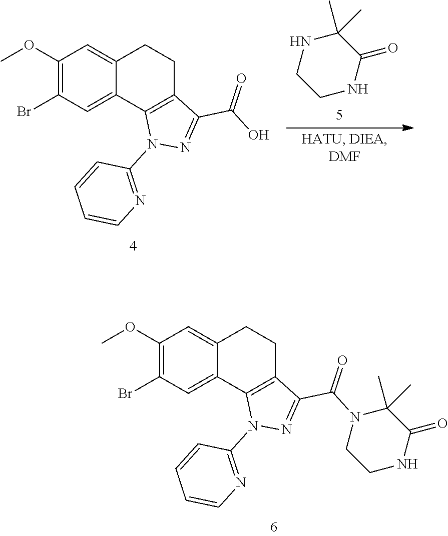

To a solution of 8-bromo-7-methoxy-1-(2-pyridyl)-4,5-dihydrobenzo[g]indazole-3-carboxylic acid (0.24 g, 599.66 μmol, 1 eq) in DMF (5 mL) was added dropwise HATU (342.01 mg, 899.49 μmol, 1.5 eq) and DIEA (232.51 mg, 1.80 mmol, 313.35 μL, 3 eq) at 25° C. After addition, the mixture was stirred at this temperature for 0.5 hr, and then 3, 3-dimethylpiperazin-2-one (92.23 mg, 719.59 μmol, 1.2 eq) was added at 25° C. The resulting mixture was stirred at 25° C. for 2 hr. The reaction mixture was poured into ice-water (20 mL) and stirred for 3 min. The aqueous phase was extracted with ethyl acetate (10 mL*3). The combined organic phase was washed with brine (20 mL), dried with anhydrous Na2SO4, filtered and concentrated in vacuum. The residue was purified by flash silica gel chromatography (ISCO®; 10 g SepaFlash® Silica Flash Column, Eluent of 0-100% Ethyl acetate/Petroleum ethergradient @50 mL/min). Compound 4-[8-bromo-7-methoxy-1-(2-pyridyl)-4,5-dihydrobenzo[g]indazole-3-carbonyl]-3,3-dimethyl-piperazin-2-one (130 mg, 254.71 μmol, 42.48% yield) was obtained as a white solid. LCMS (ESI): m/z [M+H] calcd for C24H25BrN5O3: 510.11; found: 509.9.

### Synthesis of Compound 15-1

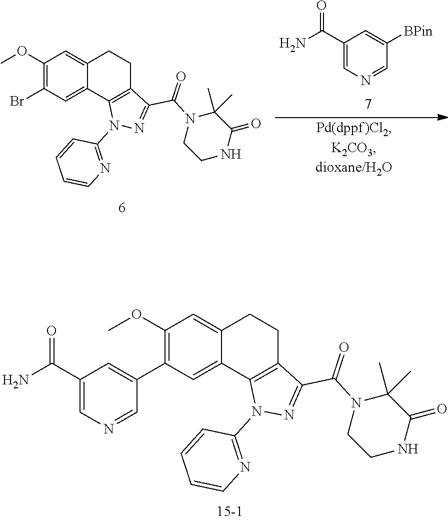

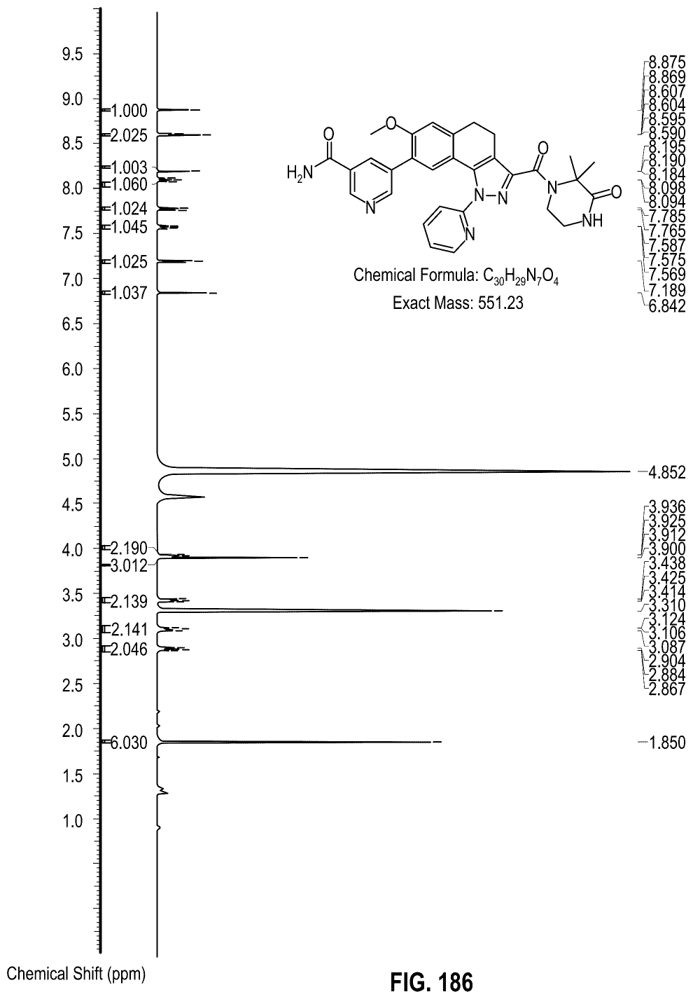

4-[8-bromo-7-methoxy-1-(2-pyridyl)-4,5-dihydrobenzo[g]indazole-3-carbonyl]-3,3-dimethyl-piperazin-2-one (60 mg, 117.56 μmol, 1 eq), 5-(4,4,5,5-tetramethyl-1,3,2-dioxaborolan-2-yl) pyridine-3-carboxamide (87.49 mg, 352.68 μmol, 3 eq), K2CO3 (32.49 mg, 235.12 μmol, 2 eq) and Pd(dppf)Cl2 (17.20 mg, 23.51 μmol, 0.2 eq) in dioxane (2 mL) and H2O (0.2 mL) was degassed and then heated to 80° C. for 2 hours under N₂. The reaction mixture was diluted with EtOAc (20 mL) and then filtered through a celite pad. The filtrate was concentrated. The residue was purified by prep-HPLC (column: Phenomenex luna C18 150*25 mm*10 um; mobile phase: [water (TFA)-ACN]; gradient: 16%-46% B over 15 min). The residue was purified by prep-HPLC (column: Waters Xbridge 150*25 mm*5 um; mobile phase: [water (NH4HCO3)-ACN]; gradient: 12%-42% B over 9 min). Compound 5-[3-(2,2-dimethyl-3-oxo-piperazine-1-carbonyl)-7-methoxy-1-(2-pyridyl)-4,5-dihydrobenzo[g]indazol-8-yl]pyridine-3-carboxamide (6 mg, 10.88 μmol, 9.25% yield) was obtained as a white solid. LCMS (ESI): m/z [M+H] calcd for C30H30N7O4: 552.23; found: 552.2. 1H NMR (400 MHz, METHANOL-d4) δ=8.87 (d, J=2.1 Hz, 1H), 8.61-8.58 (m, 2H), 8.19 (t, J=2.1 Hz, 1H), 8.10 (dt, J=1.8, 7.8 Hz, 1H), 7.77 (d, J=8.0 Hz, 1H), 7.60-7.55 (m, 1H), 7.19 (s, 1H), 6.84 (s, 1H), 3.95-3.91 (m, 2H), 3.90 (s, 3H), 3.46-3.40 (m, 2H), 3.14-3.07 (m, 2H), 2.92-2.86 (m, 2H), 1.85 (s, 6H). FIG. 186 shows the nuclear magnetic resonance of Compound 15-1.

### Synthesis of Compound 15-3

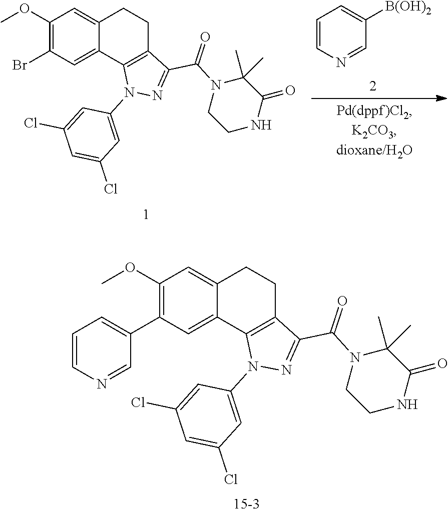 [SMILES: COc1cc2c(cc1Br)-c1c(c(C(=O)N3CCNC(=O)C3(C)C)nn1-c1cc(Cl)cc(Cl)c1)CC2 ; OB(O)c1cccnc1 ; COc1cc2c(cc1-c1cccnc1)-c1c(c(C(=O)N3CCNC(=O)C3(C)C)nn1-c1cc(Cl)cc(Cl)c1)CC2]

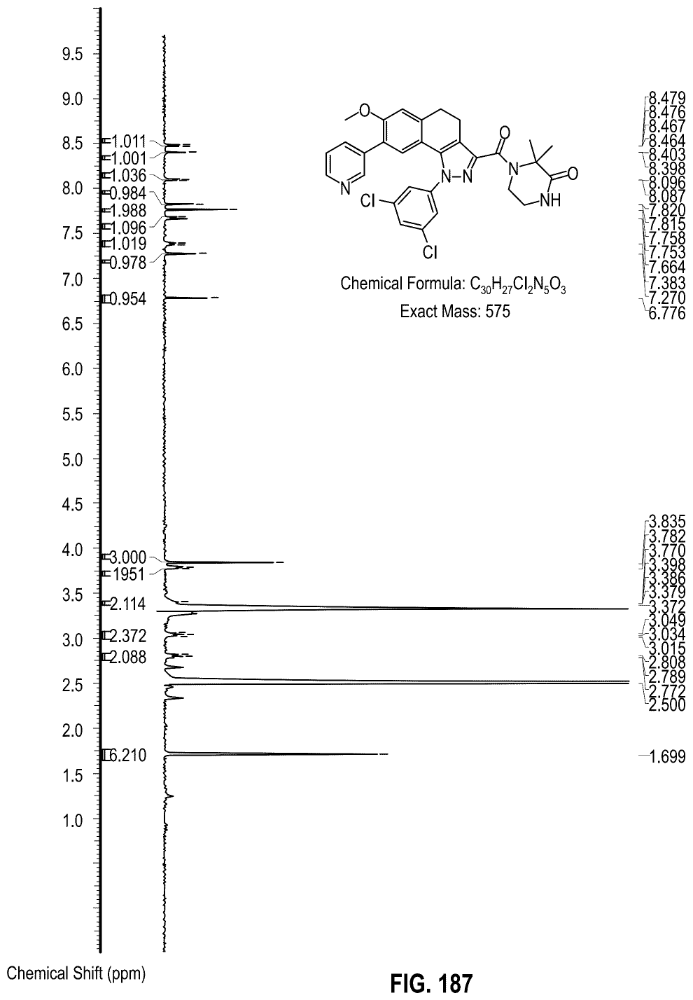

To a solution of 4-[8-bromo-1-(3,5-dichlorophenyl)-7-methoxy-4,5-dihydrobenzo [g]indazole-3-carbonyl]-3,3-dimethyl-piperazin-2-one (30 mg, 50.32 μmol, 1 eq) in dioxane (1 mL) and H2O (0.5 mL) was added 3-pyridylboronic acid (9.28 mg, 75.48 μmol, 1.5 eq) cyclopentyl(diphenyl)phosphane;dichloropalladium;iron (3.68 mg, 5.03 μmol, 0.1 eq) and K2CO3 (13.91 mg, 100.64 μmol, 2 eq). The mixture was stirred at 60° C. for 5 hr under N₂ atmosphere. The mixture was filtered and concentrated under reduced pressure to give a residue. The residue was purified by prep-HPLC (column: Welch Xtimate C18 150*25 mm*5 um;mobile phase: [water(FA)-ACN];gradient:30%-50% B over 40 min), followed by lyophilization. Compound 4-[1-(3,5-dichlorophenyl)-7-methoxy-8-(3-pyridyl)-4,5-dihydrobenzo[g]indazole-3-carbonyl]-3,3-dimethyl-piperazin-2-one (7.14 mg, 12.39 μmol, 24.61% yield, 100% purity) was obtained as a white solid. LCMS (ESI): m/z [M+H] calcd for C30H27N5O3Cl2: 576.47; found: 576.2. 1H NMR (400 MHz, DMSO-d6) δ ppm 8.47 (dd, J=4.75, 1.50 Hz, 1H) 8.40 (d, J=2.13 Hz, 1H) 8.09 (d, J=3.38 Hz, 1H) 7.84-7.80 (m, 1H) 7.76 (d, J=1.88 Hz, 2H) 7.70-7.65 (m, 1H) 7.38 (dd, J=8.00, 4.63 Hz, 1H) 7.27 (s, 1H) 6.82-6.74 (m, 1H) 3.83 (s, 3H) 3.78 (br d, J=5.13 Hz, 2H) 3.41-3.37 (m, 2H) 3.03 (br t, J=6.88 Hz, 2H) 2.84-2.76 (m, 2H) 1.70 (s, 6H). FIG. 187 shows the nuclear magnetic resonance of Compound 15-3.

### Reaction Scheme 66

### Synthesis of ethyl 2-(7-bromo-1-oxo-tetralin-2-yl)-2-oxo-acetate

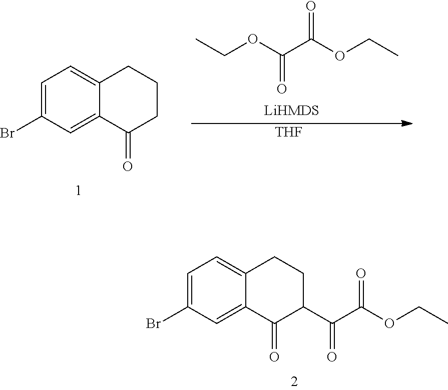

To a solution of 7-bromotetralin-1-one (3 g, 13.33 mmol, 1 eq) in THF (30 mL) was added dropwise LiHMDS (1 M, 19.99 mL, 1.5 eq) at −50° C. under N2 atmosphere, the mixture was stirred at this temperature for 0.5 hr, and then diethyl oxalate (2.53 g, 17.33 mmol, 2.37 mL, 1.3 eq) in THF (5 mL) was added dropwise at −50° C. The resulting mixture was stirred at 0° C. for 2 hr under N2 atmosphere. The reaction mixture was quenched by addition 1N HCl (30 mL) and poured into ice water (100 mL), and then extracted with ethyl acetate (100 mL*3). The combined organic layers were washed with brine (150 mL), dried over Na2SO4, filtered and concentrated under reduced pressure to give a residue. The residue was purified by column chromatography (SiO2, Petroleum ether/Ethyl acetate=1/0 to 9/1). Compound ethyl 2-(7-bromo-1-oxo-tetralin-2-yl)-2-oxo-acetate (2.1 g, 3.88 mmol, 29.07% yield, 60% purity) was obtained as a yellow oil. LCMS (ESI): m/z [M+H] calcd for C14H14O4Br: 325; found: 324.9, 326.9.

### Synthesis of ethyl 8-bromo-1-(3,5-dichlorophenyl)-4,5-dihydrobenzo[g]indazole-3-carboxylate

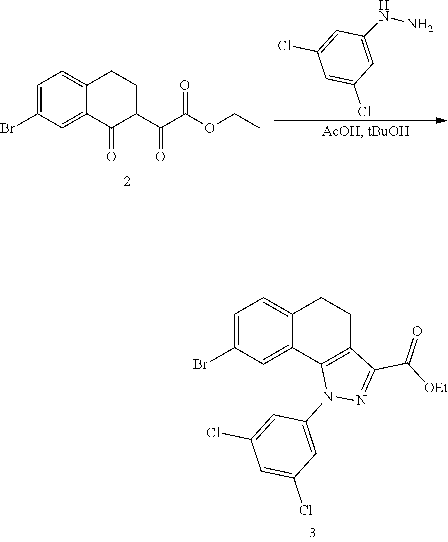

To a solution of ethyl 2-(7-bromo-1-oxo-tetralin-2-yl)-2-oxo-acetate (600 mg, 922.64 μmol, 1 eq) and (3,5-dichlorophenyl)hydrazine (200 mg, 936.80 μmol, 1.02 eq, HCl) in t-BuOH (6 mL) was added AcOH (157.34 mg, 2.62 mmol, 150.00 μL, 2.84 eq).The mixture was stirred at 90° C. for 5 hr. The mixture was cooled to room temperature and filtered, the cake was dried under reduced pressure to give the crude product. Compound ethyl 8-bromo-1-(3,5-dichlorophenyl)-4,5-dihydrobenzo[g]indazole-3-carboxylate (260 mg, 557.75 μmol, 60.45% yield) was obtained as a yellow solid. LCMS (ESI): m/z [M+H] calcd for C20H16N2BrO2Cl2: 464.97; found: 464.9, 466.9

### Synthesis of 8-bromo-1-(3,5-dichlorophenyl)-4,5-dihydrobenzo[g]indazole-3-carboxylic acid

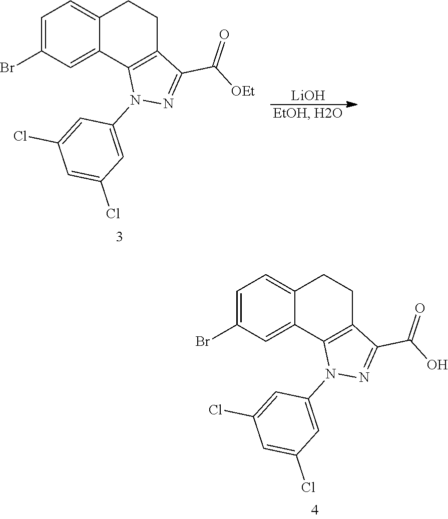

To a solution of ethyl 8-bromo-1-(3,5-dichlorophenyl)-4,5-dihydrobenzo[g]indazole-3-carboxylate (260 mg, 557.75 μmol, 1 eq) in THF (3 mL), H2O (1 mL) and was EtOH (1 mL) added LiOH·H2O (93.62 mg, 2.23 mmol, 4 eq). The mixture was stirred at 25° C. for 16 hr. The mixture was added 1N HCl (0.5 mL) to adjust the pH to 3, and the mixture was filtered to give a white solid. Compound 8-bromo-1-(3,5-dichlorophenyl)-4,5-dihydrobenzo[g]indazole-3-carboxylic acid (220 mg, 502.17 μmol, 90.03% yield) was obtained as a white solid. LCMS (ESI): m/z [M+H] calcd for C18H12N2BrO2Cl2: 436.94; found: 436.9, 438.9.

### Synthesis of 4-[8-bromo-1-(3,5-dichlorophenyl)-4,5-dihydrobenzo[g]indazole-3-carbonyl]-3,3-dimethyl-piperazin-2-one

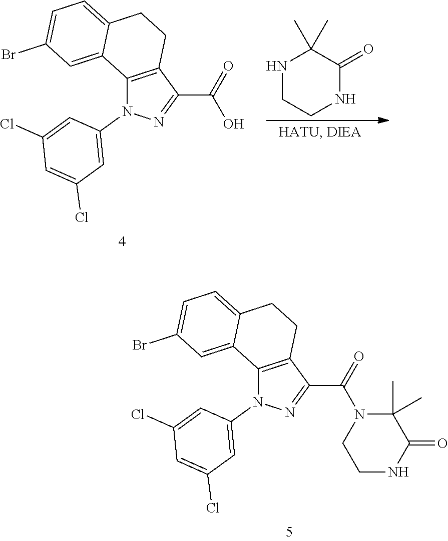

To a solution of 8-bromo-1-(3,5-dichlorophenyl)-4,5-dihydrobenzo[g]indazole-3-carboxylic acid (220 mg, 502.17 μmol, 1 eq) in DMF (3 mL) was added HATU (229.13 mg, 602.60 μmol, 1.2 eq), DIEA (194.70 mg, 1.51 mmol, 262.40 μL, 3 eq) and 3,3-dimethylpiperazin-2-one (70.80 mg, 552.38 μmol, 1.1 eq). The mixture was stirred at 25° C. for 1 hr under N2 atmosphere. The reaction mixture was diluted with H2O (10 mL) and extracted with ethyl acetate (10 mL*3). The combined organic layers were washed with brine (20 mL), dried over Na2SO4, filtered and concentrated under reduced pressure to give the crude product. Compound 4-[8-bromo-1-(3,5-dichlorophenyl)-4,5-dihydrobenzo[g]indazole-3-carbonyl]-3,3-dimethyl-piperazin-2-one (300 mg, crude) was obtained as a brown solid. LCMS (ESI): m/z [M+H] calcd for C24H22N4BrO2Cl2: 547.2; found: 546.9, 548.9.

### Synthesis of Compound 15-4

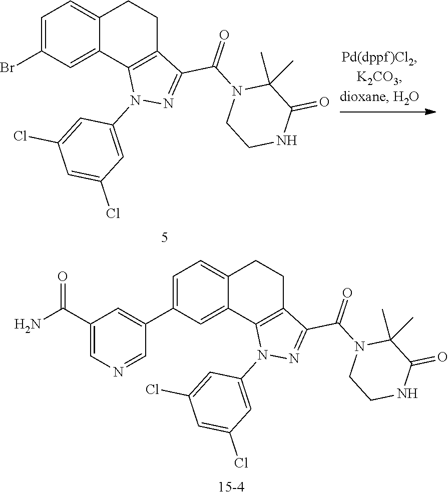 [SMILES: CC1(C)C(=O)NCCN1C(=O)c1nn(-c2cc(Cl)cc(Cl)c2)c2c1CCc1ccc(Br)cc1-2 ; CC1(C)C(=O)NCCN1C(=O)c1nn(-c2cc(Cl)cc(Cl)c2)c2c1CCc1ccc(-c3cncc(C(N)=O)c3)cc1-2]

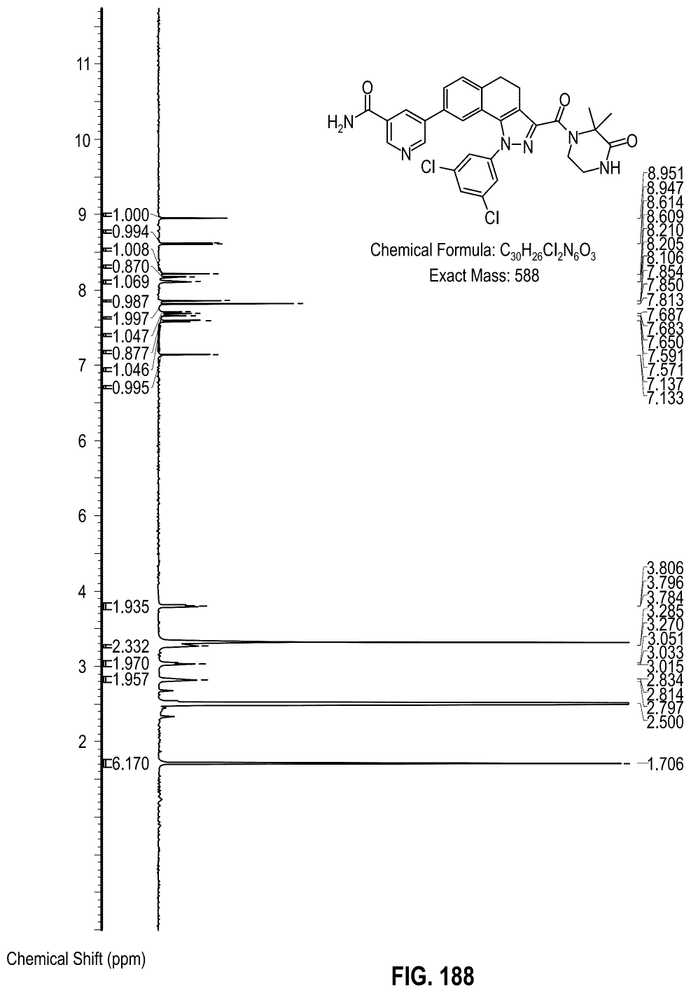

To a solution of 4-[8-bromo-1-(3,5-dichlorophenyl)-4,5-dihydrobenzo[g]indazole-3-carbonyl]-3,3-dimethyl-piperazin-2-one (50 mg, 91.20 μmol, 1 eq) in H2O (0.5 mL) and dioxane (1 mL) was added 5-(4,4,5,5-tetramethyl-1,3,2-dioxaborolan-2-yl)pyridine-3-carboxamide (33.94 mg, 136.80 μmol, 1.5 eq), K2CO3 (25.21 mg, 182.40 μmol, 2 eq) and cyclopentyl(diphenyl)phosphane;dichloropalladium;iron (6.67 mg, 9.12 μmol, 0.1 eq). The mixture was stirred at 60° C. for 16 hr under N2 atmosphere. The mixture was filtered and concentrated under reduced pressure to give a residue. The residue was purified by prep-HPLC (column: Welch Xtimate C18 150*25 mm*5 um;mobile phase: [water(FA)-ACN];gradient:35%-55% B over 40 min), followed by lyophilization. Compound 5-[1-(3,5-dichlorophenyl)-3-(2,2-dimethyl-3-oxo-piperazine-1-carbonyl)-4,5-dihydrobenzo[g]indazol-8-yl]pyridine-3-carboxamide (22.47 mg, 38.12 μmol, 41.80% yield, 100% purity) was obtained as a white solid. LCMS (ESI): m/z [M+H] calcd for C30H27N6O3Cl2: 589.14; found: 589.2. 1H NMR (400 MHz, DMSO-d6) δ=8.95 (d, J=1.88 Hz, 1H) 8.61 (d, J=2.25 Hz, 1H) 8.21 (t, J=2.00 Hz, 1H) 8.17 (s, 1H) 8.11 (br s, 1H) 7.86-7.84 (m, 1H) 7.81 (d, J=1.75 Hz, 2H) 7.69 (dd, J=8.00, 1.63 Hz, 1H) 7.65 (s, 1H) 7.58 (d, J=8.00 Hz, 1H) 7.13 (d, J=1.50 Hz, 1H) 3.85-3.75 (m, 2H) 3.28 (br d, J=5.88 Hz, 2H) 3.07-3.00 (m, 2H) 2.86-2.78 (m, 2H) 1.71 (s, 6H). FIG. 188 shows the nuclear magnetic resonance of Compound 15-4.

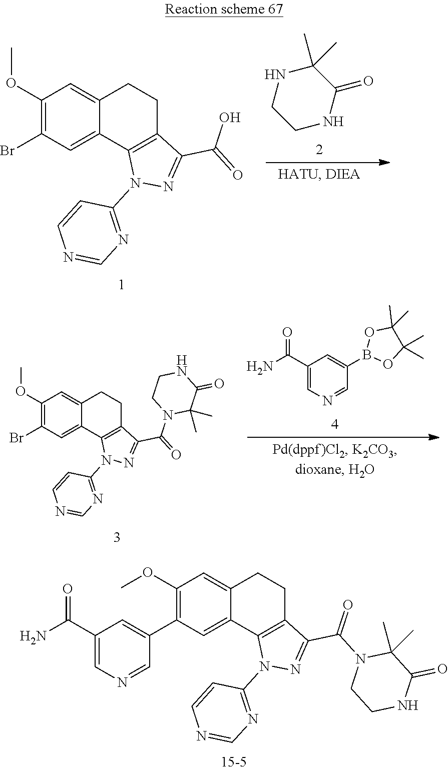

### Synthesis of 4-(8-bromo-7-methoxy-1-(pyrimidin-4-yl)-4,5-dihydro-1H-benzo[g]indazole-3-carbonyl)-3,3-dimethylpiperazin-2-one

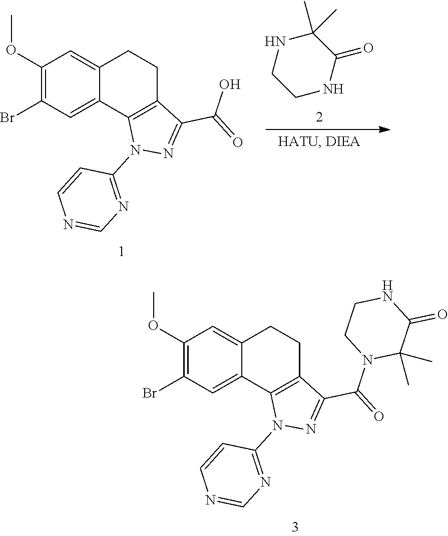

To a solution of 3,3-dimethylpiperazin-2-one (30.59 mg, 238.69 μmol, 1.2 eq) in DMF (1 mL) was added dropwise HATU (113.45 mg, 298.36 μmol, 1.5 eq) and DIEA (77.12 mg, 596.72 μmol, 103.94 μL, 3 eq) at 25° C. After addition, the mixture was stirred at this temperature for 30 min, and then 8-bromo-7-methoxy-1-(pyrimidin-4-yl)-4,5-dihydro-1H-benzo[g]indazole-3-carboxylic acid (80 mg, 199.40 mol, 1 eq) was added dropwise. The resulting mixture was stirred at 25° C. for 4 hr. The reaction mixture was acidified to pH=5 with 1 M HCl aqueous solution and diluted with H2O 20 mL and extracted with EtOAc 40 mL (20 mL*2). The combined organic layers were washed with brine 40 mL (20 mL*2), dried over Na2SO4, filtered and concentrated under reduced pressure to give a residue. The residue was purified by flash silica gel chromatography (ISCO®; 4 g SepaFlash® Silica Flash Column, Eluent of 80% Ethyl acetate/Petroleum ethergradient @30 mL/min). Compound 4-(8-bromo-7-methoxy-1-(pyrimidin-4-yl)-4,5-dihydro-1H-benzo[g]indazole-3-carbonyl)-3,3-dimethylpiperazin-2-one (70 mg, 136.89 μmol, 68.82% yield) was obtained as a white solid. 1H NMR (400 MHz, DMSO-d6) δ=9.08 (s, 1H), 9.00 (d, J=5.6 Hz, 1H), 8.14 (br s, 1H), 7.96 (dd, J=0.9, 5.4 Hz, 1H), 7.63 (s, 1H), 7.20 (s, 1H), 3.90 (s, 3H), 3.72 (br d, J=4.9 Hz, 2H), 3.28 (br d, J=2.3 Hz, 2H), 2.96-2.90 (m, 2H), 2.74-2.68 (m, 2H), 1.70 (s, 6H).

### Synthesis of Compound 15-5

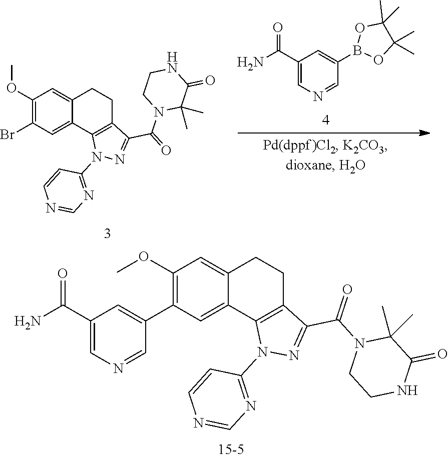 [SMILES: COc1cc2c(cc1Br)-c1c(c(C(=O)N3CCNC(=O)C3(C)C)nn1-c1ccncn1)CC2 ; CC1(C)OB(c2cncc(C(N)=O)c2)OC1(C)C ; COc1cc2c(cc1-c1cncc(C(N)=O)c1)-c1c(c(C(=O)N3CCNC(=O)C3(C)C)nn1-c1ccncn1)CC2]

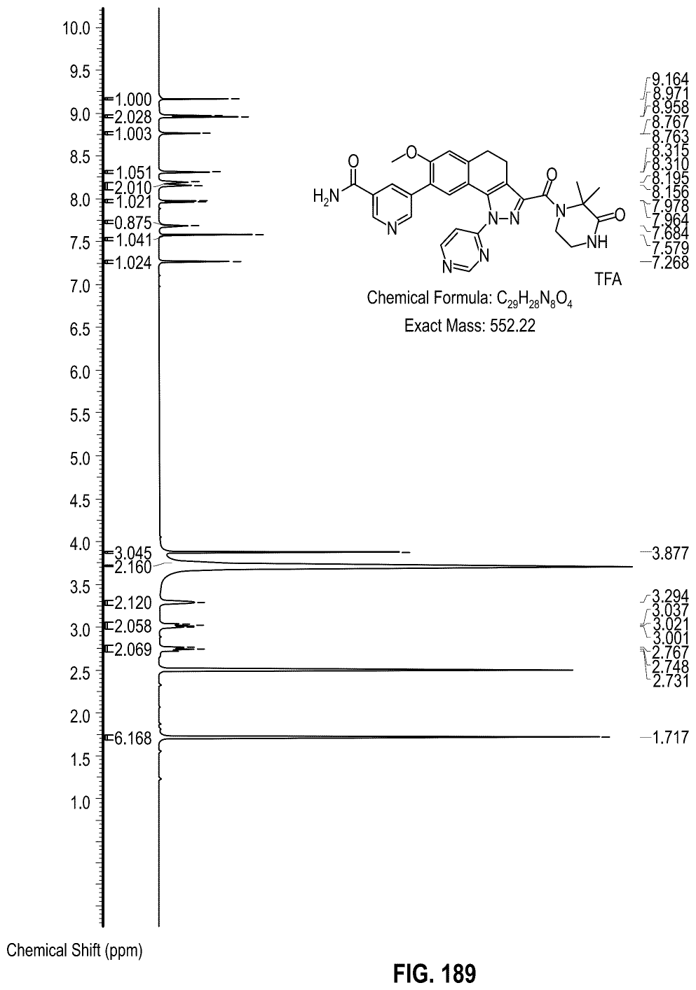

A mixture of 4-(8-bromo-7-methoxy-1-pyrimidin-4-yl-4,5-dihydrobenzo[g]indazole-3-carbonyl)-3,3-dimethyl-piperazin-2-one (40 mg, 78.22 μmol, 1 eq), 5-(4,4,5,5-tetramethyl-1,3,2-dioxaborolan-2-yl)nicotinamide (58.22 mg, 234.66 μmol, 3 eq), Pd(dppf)Cl2 (5.72 mg, 7.82 μmol, 0.1 eq) and K2CO3 (32.43 mg, 234.66 μmol, 3 eq) in dioxane (1 mL) and H2O (0.5 mL), and then the mixture was stirred at 80° C. for 16 hr under N₂ atmosphere. The reaction mixture was diluted with EtOAc(10 mL) and then filtered through a celite pad. The filtrate was concentrated. The residue was purified by prep-HPLC (TFA condition; column: Phenomenex Luna C18 150*25 mm*10 um;mobile phase: [water(TFA)-ACN];gradient:18%-48% B over 9 min). follow by lyophilization. Compound 5-(3-(2,2-dimethyl-3-oxopiperazine-1-carbonyl)-7-methoxy-1-(pyrimidin-4-yl)-4,5-dihydro-1H-benzo[g]indazol-8-yl)nicotinamide (19.11 mg, 28.38 μmol, 36.28% yield, 99% purity, TFA) was obtained as a off-white solid. LCMS (ESI): m/z [M+H] calcd for C29H29N8O4: 553.22; found: 553.2. 1H NMR (400 MHz, DMSO-d6) δ=9.16 (s, 1H), 8.98-8.95 (m, 2H), 8.76 (d, J=1.6 Hz, 1H), 8.33-8.29 (m, 1H), 8.18 (br d, J=15.5 Hz, 2H), 7.97 (d, J=5.4 Hz, 1H), 7.68 (s, 1H), 7.58 (s, 1H), 7.27 (s, 1H), 3.88 (s, 3H), 3.76-3.74 (m, 2H), 3.29 (br s, 2H), 3.02 (br t, J=7.2 Hz, 2H), 2.75 (br t, J=7.2 Hz, 2H), 1.72 (s, 6H). FIG. 189 shows the nuclear magnetic resonance of Compound 15-5.

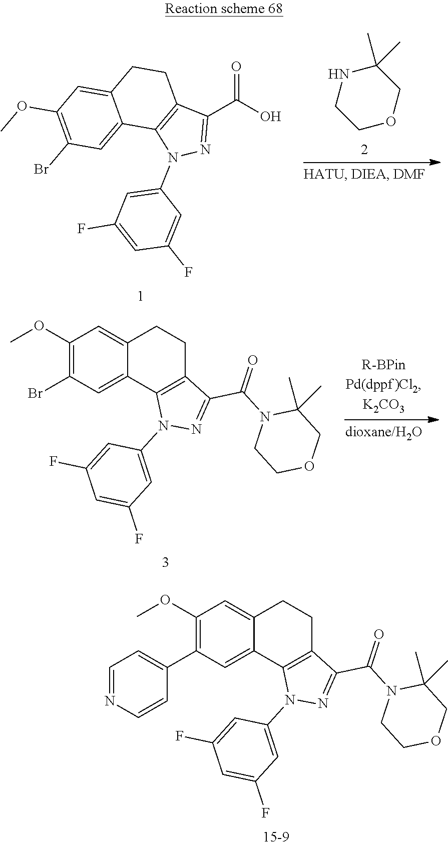

### Synthesis of [8-bromo-1-(3,5-difluorophenyl)-7-methoxy-4,5-dihydrobenzo[g]indazol-3-yl]-(3,3-dimethylmorpholin-4-yl) methanone

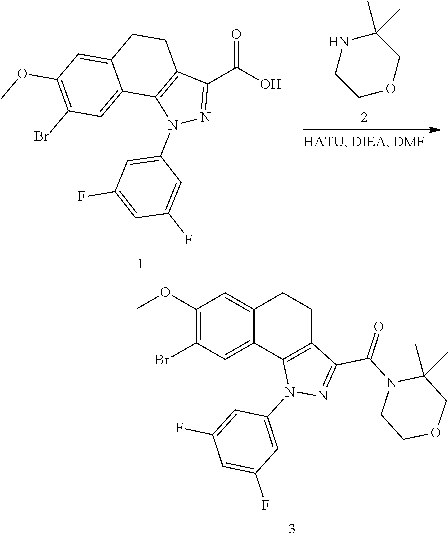

A mixture of 8-bromo-1-(3,5-difluorophenyl)-7-methoxy-4,5-dihydrobenzo[g]indazole-3-carboxylic acid (400 mg, 919.08 μmol, 1 eq), HATU (524.19 mg, 1.38 mmol, 1.5 eq) and DIEA (593.92 mg, 4.60 mmol, 800.43 μL, 5 eq) in DMF (5 mL) was stirred at 25° C. for 15 min. To the mixture was added 3,3-dimethylmorpholine (137.61 mg, 1.19 mmol, 1.3 eq). The mixture was stirred at 25° C. for 1 h. The reaction mixture was diluted with EtOAc (20 mL) and extracted with EtOAc (20 mL*3). The combined organic layers were washed with brine (30 mL*3), dried over anhydrous Na2SO4. filtered and concentrated under reduced pressure to give a residue. The residue was purified by column chromatography (SiO2, Petroleum ether/Ethyl acetate=1/0 to 3/1) to afford compound [8-bromo-1-(3,5-difluorophenyl)-7-methoxy-4,5-dihydrobenzo[g]indazol-3-yl]-(3,3-dimethylmorpholin-4-yl)methanone (436 mg, 818.97 μmol, 89.11% yield) as a white solid. LCMS (ESI): m/z [M+H] calcd for C25H25BrF2N3O3: 532.10; found: 532.1, 534.1.

### Synthesis of Compound 15-9

n

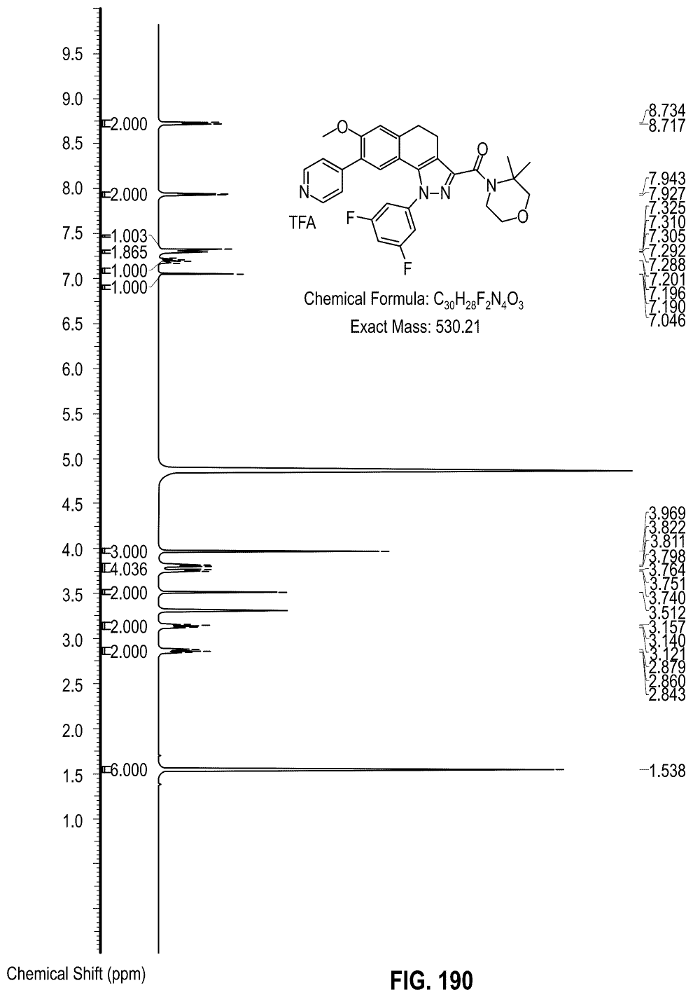

To a mixture of [8-bromo-1-(3,5-difluorophenyl)-7-methoxy-4,5-dihydrobenzo[g]indazol-3-yl]-(3,3-dimethylmorpholin-4-yl)methanone (50 mg, 93.92 μmol, 1 eq), 4-pyridylboronic acid (12.70 mg, 103.31 μmol, 1.1 eq) and K2CO3 (38.94 mg, 281.76 μmol, 3 eq) in dioxane (0.8 mL) and H2O (0.3 mL) was added Pd(dppf)Cl2 (6.87 mg, 9.39 μmol, 0.1 eq) under N2 atmosphere. The mixture was degassed and purged with N2 for 3 times, and then the mixture was stirred at 90° C. for 1 h under N2 atmosphere. The reaction mixture was diluted with H2O (10 mL) and extracted with EtOAc (10 mL*3). The combined organic layers were dried over anhydrous Na2SO4, filtered and concentrated under reduced pressure to give a residue. The crude product was purified by prep-HPLC (column: Phenomenex Luna C18 150*25 mm*10 um; mobile phase: [water (TFA)-ACN]; gradient: 22%-52% B over 9 min). Compound [1-(3,5-difluorophenyl)-7-methoxy-8-(4-pyridyl)-4, 5-dihydrobenzo[g]indazol-3-yl]-(3,3-dimethylmorpholin-4-yl)methanone (24.57 mg, 37.74 μmol, 40.18% yield, 99% purity, TFA) as a yellow solid. LCMS (ESI): m/z [M+H] calcd for C30H29F2N4O3: 531.21; found: 531.2. 1H NMR: (400 MHz, METHANOL-d4) δ=8.73 (d, J=6.6 Hz, 2H), 7.93 (d, J=6.6 Hz, 2H), 7.33 (s, 1H), 7.30 (dd, J=1.9, 7.1 Hz, 2H), 7.23-7.16 (m, 1H), 7.05 (s, 1H), 3.97 (s, 3H), 3.83-3.73 (m, 4H), 3.51 (s, 2H), 3.14 (br t, J=7.3 Hz, 2H), 2.90-2.82 (m, 2H), 1.54 (s, 6H). FIG. 190 shows the nuclear magnetic resonance of Compound 15-9.

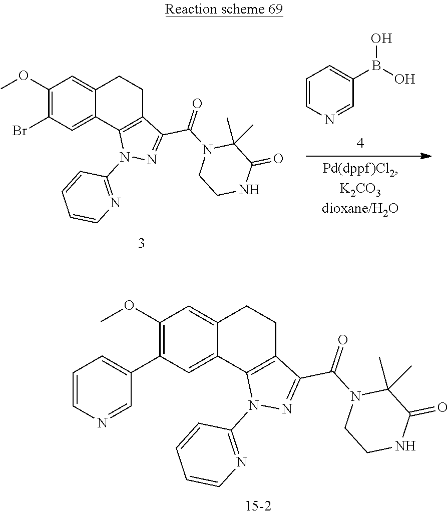 [SMILES: COc1cc2c(cc1Br)-c1c(c(C(=O)N3CCNC(=O)C3(C)C)nn1-c1ccccn1)CC2 ; OB(O)c1cccnc1 ; COc1cc2c(cc1-c1cccnc1)-c1c(c(C(=O)N3CCNC(=O)C3(C)C)nn1-c1ccccn1)CC2]

### Synthesis of Compound 15-2

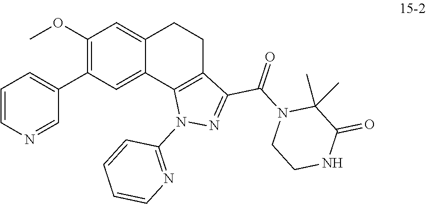 [SMILES: COc1cc2c(cc1-c1cccnc1)-c1c(c(C(=O)N3CCNC(=O)C3(C)C)nn1-c1ccccn1)CC2]

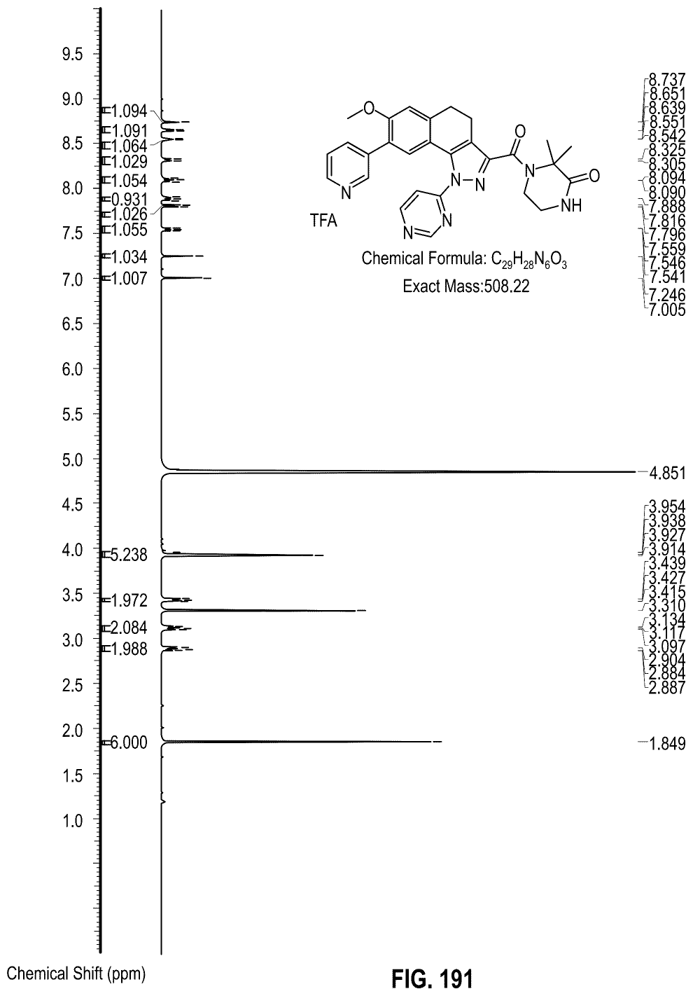

Compound 15-2 was synthesized by a procedure similar to Reaction scheme 69. LCMS (ESI): m/z [M+H] calcd for C31H30N6O5F3 509.22; found: 509.2. 1H NMR (400 MHz, METHANOL-d4) δ=8.74 (s, 1H), 8.64 (d, J=5.1 Hz, 1H), 8.57-8.49 (m, 1H), 8.32 (br d, J=8.3 Hz, 1H), 8.09 (dt, J=1.8, 7.8 Hz, 1H), 7.88 (dd, J=5.6, 8.1 Hz, 1H), 7.81 (d, J=8.0 Hz, 1H), 7.54 (dd, J=4.9, 6.8 Hz, 1H), 7.25 (s, 1H), 7.01 (s, 1H), 3.96-3.90 (m, 5H), 3.45-3.41 (m, 2H), 3.12 (t, J=7.3 Hz, 2H), 2.92-2.86 (m, 2H), 1.85 (s, 6H). FIG. 191 shows the nuclear magnetic resonance of Compound 15-2.

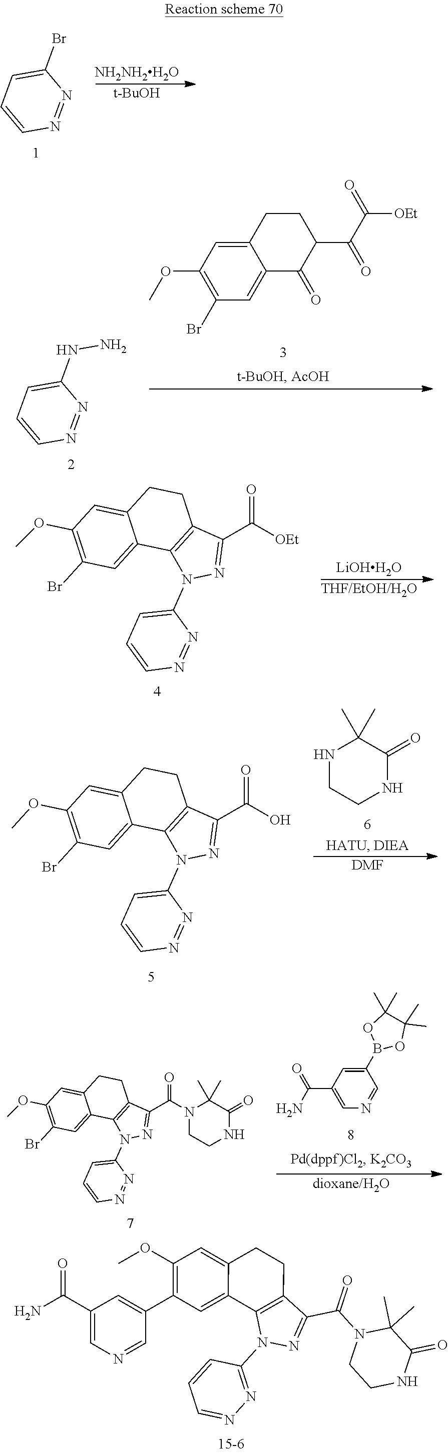

### Synthesis of Compound 15-6

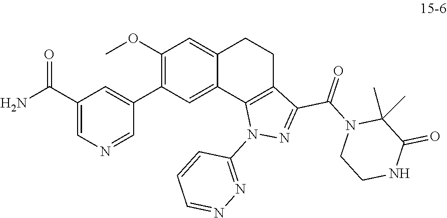 [SMILES: COc1cc2c(cc1-c1cncc(C(N)=O)c1)-c1c(c(C(=O)N3CCNC(=O)C3(C)C)nn1-c1cccnn1)CC2]

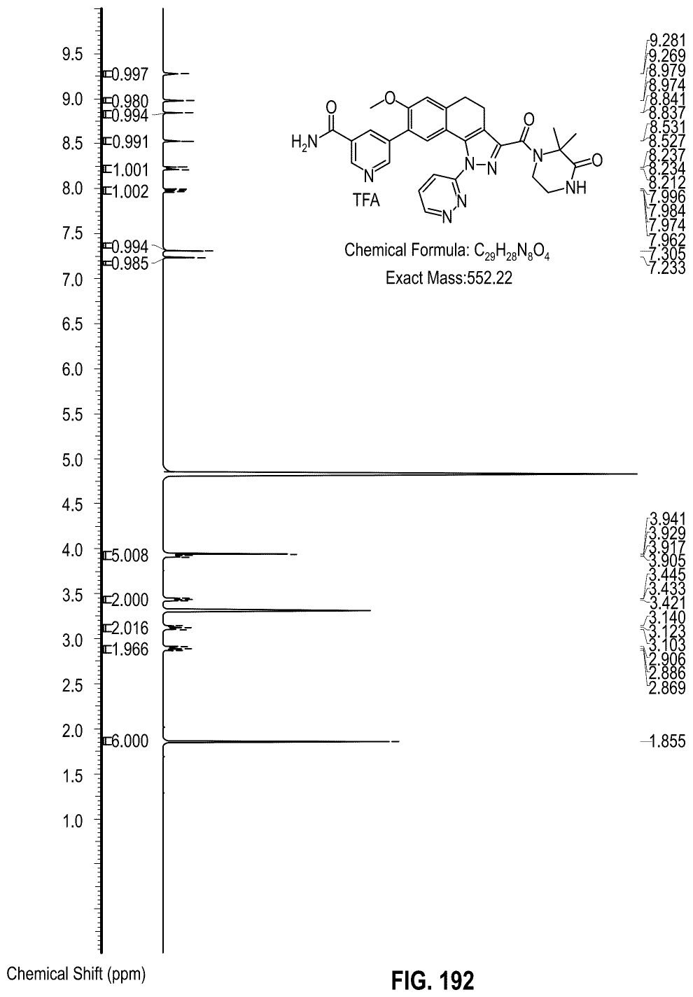

Compound 15-6 was synthesized by a procedure similar to Reaction scheme 70. LCMS (ESI): m/z [M+H] calcd for C29H29N8O4 553.22; found: 553.3. 1H NMR (400 MHz, METHANOL-d4) δ=9.28 (d, J=4.8 Hz, 1H), 8.98 (d, J=1.9 Hz, 1H), 8.84 (d, J=1.8 Hz, 1H), 8.53 (d, J=1.8 Hz, 1H), 8.22 (dd, J=1.3, 8.9 Hz, 1H), 7.98 (dd, J=4.9, 8.8 Hz, 1H), 7.30 (s, 1H), 7.23 (s, 1H), 3.97-3.88 (m, 5H), 3.46-3.40 (m, 2H), 3.12 (t, J=7.3 Hz, 2H), 2.92-2.85 (m, 2H), 1.85 (s, 6H). FIG. 192 shows the nuclear magnetic resonance of Compound 15-6.

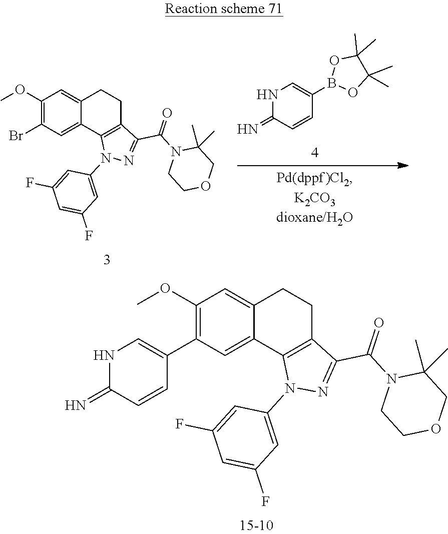

### Synthesis of Compound 15-10

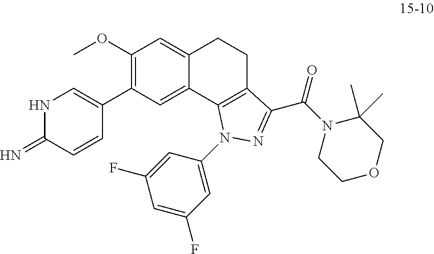 [SMILES: COc1cc2c(cc1-c1ccc(=N)[nH]c1)-c1c(c(C(=O)N3CCOCC3(C)C)nn1-c1cc(F)cc(F)c1)CC2]

Compound 15-10 was synthesized by a procedure similar to Reaction scheme 71. LCMS (ESI): m/z [M+H] calcd for C30H29F2N5O3: 545.22; found: 546.2. 1H NMR (400 MHz, METHANOL-d4) δ=7.86 (dd, J=2.2, 9.3 Hz, 1H), 7.69 (d, J=1.8 Hz, 1H), 7.30-7.23 (m, 2H), 7.21-7.15 (m, 2H), 6.98 (d, J=9.1 Hz, 1H), 6.82 (s, 1H), 3.90 (s, 3H), 3.83-3.78 (m, 2H), 3.77-3.71 (m, 2H), 3.51 (s, 2H), 3.08 (t, J=7.3 Hz, 2H), 2.86-2.78 (m, 2H), 1.53 (s, 6H). FIG. 193 shows the nuclear magnetic resonance of Compound 15-10.

### Evaluation of Novel FSHR Modulators

### Example 15. EC₅₀ of Cyclic AMP Production in CHO FSHR Cells+EC₂₀ FSH

2500 Cho-FSHR-LUC-1-1-43 cells are plated per well in 5 μl of phenol red free DMEM/F12+1% FBS. Cells are plated in 384 well, solid white low volume plates (Greiner 784075) by Multidrop. Cells are assayed by adding 100 μl of 2×EC₂₀ FSH/IBMX in DMEM/F12+0.1% BSA) by Multidrop to 2 μl of test compound stamped in 384 well plates (compounds are diluted 1:50). The final FSH concentration is 0.265 μM, and the final IBMX concentration is 200 μM. The compound plate map is as follows: Column 1: 2 μl of DMSO; Column 2: 2 μl of DMSO; Columns 3-12 and 13-24: 2 μl of test compound, diluted 1:4 in 100% DMSO, or 2 μl of FSH, diluted 1:4 in DMEM/F12+0.1% BSA. The starting concentration for FSH is 50 nM (final concentration is 0.5 nM). Furthermore, Column 23 contained 2 μl of EC₁₀₀ FSH reference (100×) (diluted in DMEM/F12+0.1% BSA) at a final concentration of 0.5 nM, and Column 24 contained 2 μl of 1 mM AS707664/2 reference compound 2.5 μl of compound+EC₂₀ FSH mixture are transferred to cell plates (1:2 dilution into 5 μl of cell media) The plates are incubated at 37° C. for 1 h. 10 μl of mixed HTRF (CisBio #62AM4PEC) reagents are added per well and incubated at room temperature for 1 h. The plates are read on Envision using the cAMP HTRF-low volume 384 well protocol. The readout is the calculated fluorescence ratio (665 nm/620 nm).

### Example 16. Rat Granulosa EC₅₀ FSH

The assay is performed pursuant to the teaching of Yanofsky et al. (2006) Allosteric activation of the follicle-stimulating hormone (FSH) receptor by selective, nonpeptide agonists (JBC 281(19): 13226-13233, which is incorporated by reference in the disclosure herein).

### Example 17. HTRF cAMP Agonist Assay Protocol (for FSHR, TSHR, LHCGR)

Mammalian expression vectors designed to express either Follicle Stimulating Hormone Receptor (FSHR), Thyroid Stimulating Hormone Receptor (TSHR), or Luteinizing Hormone/Choriogonadotropin Receptor (LHCGR), were generated with an HA-tag on the N-terminus. All constructs were expressed under a human cytomegalovirus (CMV) immediate early enhancer and promoter using the pHM6 expression vector. The protein coding region was confirmed by sequence analysis to be in-frame and of the expected sequence, matching the following accession numbers: FSHR NM_000145, TSHR NM_000369, and LHCGR NM_000233.

HTRF cAMP assays were performed using Chinese Hamster Ovary (CHO-K1) cell lines transiently expressing human FSH, TSH, and LHCG receptors. 48 hours before the assay, cells were washed with PBS and then dissociated from tissue culture flasks with 0.25% trypsin. Dissociated cells were quenched in full media (F-12K Medium Corning REF #10-025-CV, 10% Avantor Seradigm FB Essence Cat #10803-034, 1% Penicillin/Streptomycin Cytiva HyClone Cat #SV30010) and then collected by centrifugation at 290×g for 5 min. Cells were resuspended in full media and dispensed into 150 mm cell culture dishes (Nest Scientific Cat #715001), at 2.3×10⁶ cells/flask. 7 hours later, cells were transfected using Lipofectamine transfection reagent (Invitrogen Cat #18324020). Briefly, 60 μL of lipofectamine was dispensed into 2 mL of Opti-MEM (Gibco Cat #31985070) and incubated for 5 minutes at room temperature. Then, the mixture was added onto 12 g of DNA and incubated for another 20 minutes at room temperature. Cells were washed with warm (37° C.) PBS. 17.5 mL of Opti-MEM was dispensed onto the cells, followed by addition of the Opti-MEM/lipofectamine/DNA mixture. Cells were incubated overnight in a 37° C., 5% CO₂ cell incubator. 17-19 hours later, Opti-MEM was aspirated off the cells and replaced with full media. Cells were incubated for another 24 hours in a 37° C., 5% CO₂ cell incubator.

On the day of the assay, cells were washed with PBS and then dissociated from tissue culture flasks with 0.25% trypsin. Dissociated cells were quenched in full media and then collected by centrifugation at 290×g for 5 min. Cells were resuspended in PBS^{+/+} (Corning REF #20-030-CV) to a concentration of 2×10⁵ cells/mL. 10 μL/well of cells (2,000 cells/well) were dispensed into 384-well Corning plates (REF #3825), in triplicate. Cells were incubated at room temperature for 2 hours.

Example FSHR Modulator compounds were diluted in assay buffer (PBS^{+/+}, 0.05% BSA Sigma-Aldrich A3059, 500 μM IBMX Sigma-Aldrich 15879) to 3.5× the final assay concentration. 4 μL of test agonist was added to the cells and incubated for 1 hour at room temperature. 10 μL of cAMP detection reagents (LANCE Ultra cAMP Detection Kit PerkinElmer TRF0264) were dispensed onto the cells and plates were incubated for 2 hours at room temperature (cAMP detection reagents were made according to the PerkinElmer kit protocol). Plates were read on an Envision plate reader following PerkinElmer guidelines.

Raw data for all assays was transmitted directly from plate readers into a database for processing. All assay plates contained both positive and negative control wells to allow for data normalization and scaling. In agonist assays, negative control wells contained assay buffer and positive control wells contained 10 μM positive control (10 nM Follicle Stimulating Hormone Sigma-Aldrich F4021, 50 nM Thyroid Stimulating Hormone Sigma-Aldrich 869006, 50 nM Luteinizing Hormone MyBioSource MBS5308806, for their respective receptors). All control wells contained DMSO at a final assay concentration that matched the test compound wells.

In addition to buffer and acetylcholine containing negative and positive control wells, all assay plates contained an 8-point, quadruplicate cAMP standard curve. Counts from the standard curve wells were used to generate a sigmoidal standard curve, which was used to transform the raw counts from each well on the plate (scaling control wells and compound wells) into a pmol/well cAMP value. The pmol/well values for compound wells were scaled to the values in the positive and negative control wells to generate % control values. The negative control wells defined 0% response and the positive control wells defined 100% response. Data from test compound wells was scaled to these controls and are reported as % control values. Reported agonist efficacies represent the % efficacy compared to a maximal positive control response.

Percent control data were analyzed by non-linear regression with a sigmoidal dose response algorithm in the database to yield potency and efficacy data.

TABLE 2
Assay results of selected compounds via HTRF cAMP Agonist Assay showing
IC₅₀ measurements collected for selected compounds described herein for each
of FSHR (follicle stimulating hormone receptor), TSHR (thyroid stimulating
hormone receptor), and LHCGR (luteinizing hormone/choriogadotropin receptor).

| | IC50 | | | | IC50 | | | | IC50 | | | |
|---|---|---|---|---|---|---|---|---|---|---|---|---|
| | FSHR | | | | TSHR | | | | LHCGR | | | |
| Compound | LANCE | | | | LANCE | | | | LANCE | | | |
| ID | CAMP | | STDEV | n | CAMP | | STDEV | n | CAMP | | STDEV | n |
| Compound | 5.062 | nM | 0.2511 | 3 | 1091 | nM | 0.07785 | 3 | 212.7 | nM | 0.0874 | 3 |
| 10-01 | | | | | | | | | | | | |
| Compound | 40.14 | nM | 0.2111 | 3 | 2038 | nM | 0.06887 | 3 | 563.8 | nM | 0.0322 | 3 |
| 10-02 | | | | | | | | | | | | |
| Compound | 189.8 | nM | 0.2425 | 3 | 1814 | nM | 0.1279 | 4 | 3451 | nM | 0.1089 | 3 |
| 10-03 | | | | | | | | | | | | |
| Compound | 67.42 | nM | 0.1822 | 3 | 1883 | nM | 0.1124 | 4 | 210.8 | nM | 0.1078 | 3 |
| 10-06 | | | | | | | | | | | | |
| Compound | 22.5 | nM | 0.1371 | 3 | 6793 | nM | 0.109 | 3 | 1156 | nM | 0.1889 | 3 |
| 10-07 | | | | | | | | | | | | |
| Compound | 399.5 | nM | 0.13 | 3 | 100000 | nM | 0 | 3 | 4536 | nM | 0.1121 | 3 |
| 10-08 | | | | | | | | | | | | |
| Compound | 164.8 | nM | 0.04544 | 3 | 8239 | nM | 0.2068 | 3 | 1826 | nM | 0.0131 | 3 |
| 10-09 | | | | | | | | | | | | |
| Compound | 4.172 | nM | 0.2987 | 8 | 28.75 | nM | 0.1884 | 3 | 45.02 | nM | 0.2943 | 2 |
| 1-01 | | | | | | | | | | | | |
| Compound | 14.82 | nM | 0.09516 | 3 | 2384 | nM | 0.02633 | 3 | 1674 | nM | 0.027 | 3 |
| 10-10 | | | | | | | | | | | | |
| Compound | 9.546 | nM | 0.2266 | 8 | 61.93 | nM | 0.2421 | 3 | 87.26 | nM | 0.3813 | 2 |
| 1-02 | | | | | | | | | | | | |
| Compound | 14.76 | nM | 0.2663 | 8 | 105.1 | nM | 0.2177 | 3 | 80.17 | nM | 0.3021 | 2 |
| 1-02A | | | | | | | | | | | | |
| Compound | 18.16 | nM | 0.3314 | 9 | 258.1 | nM | 0.2272 | 3 | 116.9 | nM | 0.3218 | 2 |
| 1-03 | | | | | | | | | | | | |
| Compound | 51.87 | nM | 0.2005 | 8 | 322.5 | nM | 0.2108 | 3 | 158.6 | nM | 0.2141 | 2 |
| 1-04 | | | | | | | | | | | | |
| Compound | 767.9 | nM | 0.1458 | 8 | 805.7 | nM | 0.03052 | 3 | 1013 | nM | 0.014 | 2 |
| 1-05 | | | | | | | | | | | | |
| Compound | 1110 | nM | 0.1952 | 7 | 641.2 | nM | 0.1571 | 5 | 1143 | nM | 0.2091 | 4 |
| 1-06 | | | | | | | | | | | | |
| Compound | 12.53 | nM | 0.2445 | 3 | 378.8 | nM | 0.07656 | 3 | 326 | nM | 0.2091 | 3 |
| 11-01 | | | | | | | | | | | | |
| Compound | 50.12 | nM | 0.1533 | 8 | 819.9 | nM | 0.4255 | 6 | 1230 | nM | 0.5523 | 8 |
| 11-01A | | | | | | | | | | | | |
| Compound | 41.95 | nM | 0.1668 | 3 | 1050 | nM | 0.1943 | 3 | 367.1 | nM | 0.2878 | 3 |
| 11-02 | | | | | | | | | | | | |
| Compound | 74.96 | nM | 0.1496 | 3 | 648.6 | nM | 0.13 | 3 | 778.3 | nM | 0.1028 | 3 |
| 11-03 | | | | | | | | | | | | |
| Compound | 14.79 | nM | 0.2816 | 3 | 1336 | nM | 0.2324 | 4 | 89.55 | nM | 0.2715 | 3 |
| 12-01 | | | | | | | | | | | | |
| Compound | 5.073 | nM | 0.1466 | 3 | 32520 | nM | 0.2171 | 3 | 374.1 | nM | 0.204 | 3 |
| 12-02 | | | | | | | | | | | | |
| Compound | 5.09 | nM | 0.2513 | 3 | 1012 | nM | 0.02134 | 3 | 73.13 | nM | 0.2275 | 3 |
| 12-04 | | | | | | | | | | | | |
| Compound | 15.41 | nM | 0.09223 | 3 | 100000 | nM | 0 | 3 | 100000 | nM | 0 | 3 |
| 12-05 | | | | | | | | | | | | |
| Compound | 21.23 | nM | 0.107 | 3 | 1611 | nM | 0.1298 | 3 | 730.4 | nM | 0.1035 | 3 |
| 12-07 | | | | | | | | | | | | |
| Compound | 25.11 | nM | 0.34 | 4 | 79550 | nM | 0.1721 | 3 | 107800 | nM | 0.0566 | 3 |
| 12-09 | | | | | | | | | | | | |
| Compound | 26.55 | nM | 0.1323 | 3 | 358.4 | nM | 0.09461 | 3 | 539.1 | nM | 0.0743 | 3 |
| 12-11 | | | | | | | | | | | | |
| Compound | 100000 | nM | 0 | 3 | 100000 | nM | 0 | 3 | 100000 | nM | 0 | 3 |
| 12-12 | | | | | | | | | | | | |
| Compound | 228.1 | nM | 0.1407 | 3 | 100000 | nM | 0 | 3 | 100000 | nM | 0 | 3 |
| 12-12 | | | | | | | | | | | | |
| Compound | 4.983 | nM | 0.2462 | 4 | 2742 | nM | 0.2034 | 5 | 472.4 | nM | 0.1561 | 5 |
| 12-13 | | | | | | | | | | | | |
| Compound | 3.088 | nM | 0.3228 | 3 | 996.4 | nM | 0.1246 | 3 | 129.1 | nM | 0.0397 | 3 |
| 12-15 | | | | | | | | | | | | |
| Compound | 20.9 | nM | 0.2651 | 5 | 3050 | nM | 0.31 | 5 | 979.1 | nM | 0.1509 | 5 |
| 12-16 | | | | | | | | | | | | |
| Compound | 66.85 | nM | 0.1604 | 6 | 3400 | nM | 0.1682 | 5 | 1250 | nM | 0.1341 | 6 |
| 12-18 | | | | | | | | | | | | |
| Compound | 41.29 | nM | 0.3271 | 3 | 100000 | nM | 0 | 3 | 15200 | nM | 0.2167 | 3 |
| 12-19 | | | | | | | | | | | | |
| Compound | 141.7 | nM | 0.1406 | 4 | 100000 | nM | 0 | 4 | 100000 | nM | 0 | 4 |
| 12-21 | | | | | | | | | | | | |
| Compound | 70.83 | nM | 0.3166 | 2 | 5883 | nM | 0.184 | 2 | 359.5 | nM | 0.0592 | 2 |
| 12-22 | | | | | | | | | | | | |
| Compound | 36.71 | nM | 0.2323 | 3 | 7756 | nM | 0.2646 | 3 | 871.7 | nM | 0.1316 | 3 |
| 12-23 | | | | | | | | | | | | |
| Compound | 6736 | nM | 0.08992 | 3 | 100000 | nM | 0 | 3 | 7695 | nM | 0.1508 | 3 |
| 13-01 | | | | | | | | | | | | |
| Compound | 100000 | nM | 0 | 3 | 100000 | nM | 0 | 3 | 8570 | nM | 0.1251 | 3 |
| 13-01 | | | | | | | | | | | | |
| Compound | 100000 | nM | 0 | 3 | 100000 | nM | 0 | 3 | 100000 | nM | 0 | 3 |
| 13-02 | | | | | | | | | | | | |
| Compound | 100000 | nM | 0 | 5 | 100000 | nM | 0 | 5 | 100000 | nM | 0 | 5 |
| 13-04 | | | | | | | | | | | | |
| Compound | 100000 | nM | 0 | 3 | 100000 | nM | 0 | 3 | 100000 | nM | 0 | 3 |
| 13-05 | | | | | | | | | | | | |
| Compound | 100000 | nM | 0 | 3 | 100000 | nM | 0 | 3 | 100000 | nM | 0 | 3 |
| 13-07 | | | | | | | | | | | | |
| Compound | 100000 | nM | 0 | 3 | 100000 | nM | 0 | 3 | 100000 | nM | 0 | 3 |
| 13-08 | | | | | | | | | | | | |
| Compound | 2128 | nM | 0.1896 | 2 | 2293 | nM | 0.6827 | 2 | 70160 | nM | 0.0748 | 2 |
| 13-09 | | | | | | | | | | | | |
| Compound | 13820 | nM | 0.03163 | 2 | 100000 | nM | 0 | 3 | 100000 | nM | 0 | 3 |
| 14-03 | | | | | | | | | | | | |
| Compound | 75.79 | nM | 0.3149 | 3 | 100000 | nM | 0 | 4 | 1091 | nM | 0.3784 | 4 |
| 15-01 | | | | | | | | | | | | |
| Compound | 477.3 | nM | 0.2431 | 3 | 100000 | nM | 0 | 3 | 23530 | nM | 0.1412 | 3 |
| 15-02 | | | | | | | | | | | | |
| Compound | 31.14 | nM | 0.1489 | 3 | 2016 | nM | 0.1905 | 3 | 462.2 | nM | 0.0995 | 3 |
| 15-03 | | | | | | | | | | | | |
| Compound | 381.2 | nM | 0.01571 | 3 | 4835 | nM | 0.1494 | 3 | 1037 | nM | 0.1252 | 3 |
| 15-04 | | | | | | | | | | | | |
| Compound | 363 | nM | 0.2394 | 3 | 100000 | nM | 0 | 3 | 45130 | nM | 0.3091 | 3 |
| 15-05 | | | | | | | | | | | | |
| Compound | 3622 | nM | 0.07593 | 3 | 100000 | nM | 0 | 3 | 75230 | nM | 0.2141 | 3 |
| 15-06 | | | | | | | | | | | | |
| Compound | 7.323 | nM | 0.1932 | 2 | 161.7 | nM | 0.2004 | 2 | 41.23 | nM | 0.1957 | 2 |
| 15-08 | | | | | | | | | | | | |
| Compound | 370 | nM | 0.2984 | 3 | 2179 | nM | 0.1959 | 3 | 541.4 | nM | 0.055 | 3 |
| 15-09 | | | | | | | | | | | | |
| Compound | 21.15 | nM | 0.2102 | 3 | 172.5 | nM | 0.04571 | 3 | 107.2 | nM | 0.1262 | 3 |
| 15-10 | | | | | | | | | | | | |
| Compound | 3.441 | nM | 0.4646 | 7 | 1296 | nM | 0.1041 | 6 | 26.8 | nM | 0.1542 | 7 |
| 2-01 | | | | | | | | | | | | |
| Compound | 1.979 | nM | 0.4663 | 6 | 581.8 | nM | 0.243 | 8 | 37.34 | nM | 0.3509 | 8 |
| 2-02 | | | | | | | | | | | | |
| Compound | 21.98 | nM | 0.1239 | 3 | 454.8 | nM | 0.2104 | 4 | 53.58 | nM | 0.2977 | 4 |
| 2-03 | | | | | | | | | | | | |
| Compound | 7.917 | nM | 0.3666 | 4 | 412 | nM | 0.2872 | 5 | 39.57 | nM | 0.2672 | 5 |
| 2-04 | | | | | | | | | | | | |
| Compound | 637.5 | nM | 0.1958 | 5 | 37100 | nM | 0.2716 | 5 | 853 | nM | 0.1604 | 5 |
| 2-05 | | | | | | | | | | | | |
| Compound | 194.7 | nM | 0.3144 | 5 | 9828 | nM | 0.3952 | 5 | 1943 | nM | 0.1405 | 5 |
| 2-06 | | | | | | | | | | | | |
| Compound | 2407 | nM | 0.1622 | 4 | 100000 | nM | 0 | 4 | 4491 | nM | 0.2285 | 5 |
| 2-07 | | | | | | | | | | | | |
| Compound | 475 | nM | 0.312 | 6 | 42100 | nM | 0.3833 | 7 | 1818 | nM | 0.3148 | 7 |
| 2-08 | | | | | | | | | | | | |
| Compound | 6.718 | nM | 0.2128 | 4 | 358.6 | nM | 0.111 | 5 | 95.03 | nM | 0.173 | 5 |
| 3-01 | | | | | | | | | | | | |
| Compound | 3.917 | nM | 0.3723 | 7 | 155.7 | nM | 0.1626 | 8 | 63.8 | nM | 0.1821 | 8 |
| 3-02 | | | | | | | | | | | | |
| Compound | 325.2 | nM | 0.05891 | 3 | 2222 | nM | 0.1528 | 4 | 415.2 | nM | 0.2088 | 4 |
| 3-03 | | | | | | | | | | | | |
| Compound | 223 | nM | 0.03313 | 3 | 3090 | nM | 0.1592 | 4 | 847.5 | nM | 0.2791 | 4 |
| 3-04 | | | | | | | | | | | | |
| Compound | 55.68 | nM | 0.1648 | 3 | 66870 | nM | 0.3495 | 4 | 174.9 | nM | 0.1239 | 3 |
| 3-05 | | | | | | | | | | | | |
| Compound | 43.36 | nM | 0.1429 | 3 | 962.1 | nM | 0.1079 | 3 | 390.3 | nM | 0.0979 | 3 |
| 3-06 | | | | | | | | | | | | |
| Compound | 4564 | nM | 0.2841 | 2 | 100000 | nM | 0 | 3 | 39000 | nM | 0.2773 | 2 |
| 3-07 | | | | | | | | | | | | |
| Compound | 577.5 | nM | 0.2049 | 3 | 100000 | nM | 0 | 3 | 7189 | nM | 0.1136 | 3 |
| 3-08 | | | | | | | | | | | | |
| Compound | 11.87 | nM | 0.158 | 6 | 2227 | nM | 0.3215 | 5 | 262.2 | nM | 0.1806 | 6 |
| 3-09 | | | | | | | | | | | | |
| Compound | 2.909 | nM | 0.3842 | 5 | 271.3 | nM | 0.2343 | 5 | 71.04 | nM | 0.3388 | 5 |
| 3-10 | | | | | | | | | | | | |
| Compound | 76.35 | nM | 0.2613 | 6 | 127400 | nM | 0.3323 | 3 | 1172 | nM | 0.1648 | 7 |
| 3-10A | | | | | | | | | | | | |
| Compound | 375.5 | nM | 0.178 | 6 | 100000 | nM | 0 | 7 | 5232 | nM | 0.3917 | 7 |
| 3-11 | | | | | | | | | | | | |
| Compound | 319.4 | nM | 0.2829 | 6 | 1735 | nM | 0.3851 | 5 | 5698 | nM | 0.3741 | 6 |
| 3-12 | | | | | | | | | | | | |
| Compound | 100000 | nM | 0 | 3 | 1144 | nM | 0.1701 | 4 | 2725 | nM | 0.1455 | 4 |
| 4-01 | | | | | | | | | | | | |
| Compound | 100000 | nM | 0 | 3 | 100000 | nM | 0 | 2 | 100000 | nM | 0 | 2 |
| 4-01A | | | | | | | | | | | | |
| Compound | 100000 | nM | 0 | 3 | 1951 | nM | 0.09822 | 4 | 2925 | nM | 0.1894 | 4 |
| 4-02 | | | | | | | | | | | | |
| Compound | 100000 | nM | 0 | 2 | 100000 | nM | 0 | 2 | 100000 | nM | 0 | 2 |
| 4-02A | | | | | | | | | | | | |
| Compound | 579 | nM | 0.05326 | 4 | 786.6 | nM | 0.2173 | 3 | 69.43 | nM | 0.3458 | 4 |
| 4-03 | | | | | | | | | | | | |
| Compound | 1861 | nM | 0.1813 | 4 | 9080 | nM | 0.1783 | 3 | 100000 | nM | 0 | 4 |
| 4-03A | | | | | | | | | | | | |
| Compound | 2357 | nM | 0.2127 | 4 | 1765 | nM | 0.1471 | 3 | 643.7 | nM | 0.2204 | 4 |
| 4-03B | | | | | | | | | | | | |
| Compound | 585.7 | nM | 0.143 | 4 | 2744 | nM | 0.1168 | 3 | 165.2 | nM | 0.2359 | 4 |
| 4-04 | | | | | | | | | | | | |
| Compound | 1364 | nM | 0.2382 | 4 | 25750 | nM | 0.1761 | 3 | 100000 | nM | 0 | 4 |
| 4-04A | | | | | | | | | | | | |
| Compound | 100000 | nM | 0 | 5 | 100000 | nM | 0 | 5 | 4085 | nM | 0.1949 | 4 |
| 4-05 | | | | | | | | | | | | |
| Compound | 100000 | nM | 0 | 5 | 100000 | nM | 0 | 3 | 100000 | nM | 0 | 3 |
| 4-05A | | | | | | | | | | | | |
| Compound | 100000 | nM | 0 | 3 | 100000 | nM | 0 | 2 | 100000 | nM | 0 | 2 |
| 4-06 | | | | | | | | | | | | |
| Compound | 100000 | nM | 0 | 3 | 100000 | nM | 0 | 2 | 100000 | nM | 0 | 2 |
| 4-06A | | | | | | | | | | | | |
| Compound | 2811 | nM | 0.03984 | 2 | 10370 | nM | 0.2413 | 2 | 1297 | nM | 0.3654 | 2 |
| 4-07 | | | | | | | | | | | | |
| Compound | 100000 | nM | 0 | 2 | 100000 | nM | 0 | 2 | 100000 | nM | 0 | 2 |
| 4-07A | | | | | | | | | | | | |
| Compound | 3013 | nM | 0.1031 | 6 | 5790 | nM | 0.3259 | 4 | 1576 | nM | 0.1137 | 6 |
| 4-08 | | | | | | | | | | | | |
| Compound | 4888 | nM | 0.3858 | 5 | 57990 | nM | 0.2239 | 4 | 100000 | nM | 0 | 5 |
| 4-08A | | | | | | | | | | | | |
| Compound | 14.81 | nM | 0.2244 | 7 | 79.85 | nM | 0.3876 | 8 | 46.74 | nM | 0.3992 | 8 |
| 5-01 | | | | | | | | | | | | |
| Compound | 196.6 | nM | 0.1308 | 3 | 293.6 | nM | 0.1328 | 4 | 391.1 | nM | 0.2252 | 4 |
| 5-02 | | | | | | | | | | | | |
| Compound | 4.641 | nM | 0.2732 | 3 | 17.6 | nM | 0.3835 | 4 | 46.23 | nM | 0.1865 | 4 |
| 5-03 | | | | | | | | | | | | |
| Compound | 8.197 | nM | 0.3398 | 5 | 56.51 | nM | 0.3426 | 6 | 34.16 | nM | 0.3034 | 6 |
| 5-04 | | | | | | | | | | | | |
| Compound | 558.5 | nM | 0.1831 | 5 | 988.4 | nM | 0.1617 | 5 | 711.8 | nM | 0.1325 | 5 |
| 5-05 | | | | | | | | | | | | |
| Compound | 494 | nM | 0.1452 | 3 | 794.6 | nM | 0.1392 | 3 | 599.2 | nM | 0.1183 | 3 |
| 5-06 | | | | | | | | | | | | |
| Compound | 115 | nM | 0.1344 | 3 | 1042 | nM | 0.08295 | 4 | 256.5 | nM | 0.156 | 4 |
| 5-07 | | | | | | | | | | | | |
| Compound | 195.1 | nM | 0.09038 | 3 | 1842 | nM | 0.1183 | 4 | 298.8 | nM | 0.0614 | 4 |
| 5-08 | | | | | | | | | | | | |
| Compound | 5.687 | nM | 0.1809 | 5 | 135.5 | nM | 0.2379 | 3 | 61.05 | nM | 0.2273 | 3 |
| 6-01 | | | | | | | | | | | | |
| Compound | 1087 | nM | 0.16 | 7 | 1423 | nM | 0.02264 | 3 | 3426 | nM | 0.0102 | 2 |
| 6-01A | | | | | | | | | | | | |
| Compound | 33.33 | nM | 0.2123 | 8 | 701.4 | nM | 0.1765 | 5 | 276.7 | nM | 0.2338 | 4 |
| 6-01B | | | | | | | | | | | | |
| Compound | 8.528 | nM | 0.182 | 6 | 216.4 | nM | 0.114 | 3 | 98.81 | nM | 0.0992 | 4 |
| 6-02 | | | | | | | | | | | | |
| Compound | 861 | nM | 0.244 | 8 | 1539 | nM | 0.1427 | 5 | 2355 | nM | 0.1661 | 4 |
| 6-02A | | | | | | | | | | | | |
| Compound | 31.92 | nM | 0.3342 | 8 | 949.5 | nM | 0.1851 | 5 | 244.6 | nM | 0.1599 | 4 |
| 6-02B | | | | | | | | | | | | |
| Compound | 162.7 | nM | 0.1592 | 9 | 2305 | nM | 0.09005 | 3 | 385.5 | nM | 0.1579 | 2 |
| 6-03 | | | | | | | | | | | | |
| Compound | 179.8 | nM | 0.2719 | 10 | 3871 | nM | 0.3355 | 3 | 784 | nM | 0.0362 | 2 |
| 6-04 | | | | | | | | | | | | |
| Compound | 5.239 | nM | 0.3777 | 7 | 14.18 | nM | 0.4423 | 8 | 28.18 | nM | 0.2739 | 8 |
| 6-05 | | | | | | | | | | | | |
| Compound | 13.58 | nM | 0.2892 | 5 | 92.71 | nM | 0.1874 | 4 | 268.7 | nM | 0.1517 | 5 |
| 6-06 | | | | | | | | | | | | |
| Compound | 83.17 | nM | 0.1361 | 7 | 508.4 | nM | 0.1154 | 3 | 362 | nM | 0.1867 | 2 |
| 6-07 | | | | | | | | | | | | |
| Compound | 78.38 | nM | 0.1947 | 5 | 450.4 | nM | 0.08497 | 3 | 300.3 | nM | 0.0829 | 3 |
| 6-08 | | | | | | | | | | | | |
| Compound | 20.85 | nM | 0.3663 | 3 | 3845 | nM | 0.182 | 2 | 310.9 | nM | 0.1304 | 3 |
| 8-01 | | | | | | | | | | | | |
| Compound | 30.52 | nM | 0.255 | 2 | 1085 | nM | 0.1858 | 2 | 486.3 | nM | 0.1399 | 2 |
| 8-02 | | | | | | | | | | | | |
| Compound | 449.1 | nM | 0.1349 | 2 | 2248 | nM | 0.002378 | 2 | 9883 | nM | 0.1749 | 2 |
| 8-03 | | | | | | | | | | | | |
| Compound | 28.3 | nM | 0.1552 | 3 | 2192 | nM | 0.1434 | 3 | 1079 | nM | 0.1453 | 3 |
| 8-04 | | | | | | | | | | | | |
| Compound | 9.795 | nM | 0.2845 | 3 | 906.2 | nM | 0.08214 | 3 | 478.7 | nM | 0.0605 | 3 |
| 8-04A | | | | | | | | | | | | |
| Compound | 60.26 | nM | 0.1777 | 4 | 2572 | nM | 0.07949 | 3 | 116.2 | nM | 0.2651 | 4 |
| 8-05 | | | | | | | | | | | | |
| Compound | 17.57 | nM | 0.1601 | 4 | 297.4 | nM | 0.3722 | 4 | 235.5 | nM | 0.3584 | 4 |
| 8-06 | | | | | | | | | | | | |
| Compound | 115.2 | nM | 0.2702 | 4 | 9952 | nM | 0.3269 | 4 | 949.2 | nM | 0.3607 | 4 |
| 8-07 | | | | | | | | | | | | |
| Compound | 3043 | nM | 0.1955 | 5 | 100000 | nM | 0 | 4 | 7521 | nM | 0.0766 | 3 |
| 8-07A | | | | | | | | | | | | |
| Compound | 233.7 | nM | 0.06567 | 3 | 3128 | nM | 0.1369 | 3 | 1238 | nM | 0.0346 | 3 |
| 8-08 | | | | | | | | | | | | |
| Compound | 4.435 | nM | 0.3863 | 5 | 1570 | nM | 0.06871 | 5 | 182.5 | nM | 0.2542 | 5 |
| 8-09 | | | | | | | | | | | | |
| Compound | 991.3 | nM | 0.152 | 4 | 100000 | nM | 0 | 4 | 1139 | nM | 0.1139 | 4 |
| 8-10 | | | | | | | | | | | | |
| Compound | 244 | nM | 0.03257 | 3 | 3362 | nM | 0.06571 | 3 | 1415 | nM | 0.0938 | 3 |
| 8-13 | | | | | | | | | | | | |
| Compound | 3.47 | nM | 0.209 | 4 | 255.4 | nM | 0.1452 | 3 | 129 | nM | 0.2592 | 4 |
| 8-14 | | | | | | | | | | | | |
| Compound | 33.2 | nM | 0.06766 | 4 | 10250 | nM | 0.3255 | 4 | 59.65 | nM | 0.1178 | 4 |
| 8-15 | | | | | | | | | | | | |
| Compound | 70.38 | nM | 0.1997 | 3 | 4662 | nM | 0.3117 | 3 | 784.6 | nM | 0.1902 | 3 |
| 8-16 | | | | | | | | | | | | |
| Compound | 79.33 | nM | 0.2474 | 4 | 100000 | nM | 0 | 3 | 4402 | nM | 0.2077 | 4 |
| 8-16A | | | | | | | | | | | | |
| Compound | 769.2 | nM | 0.1878 | 3 | 18910 | nM | 0.1301 | 3 | 29910 | nM | 0.3495 | 4 |
| 8-16B | | | | | | | | | | | | |
| Compound | 114.8 | nM | 0.2356 | 4 | 6033 | nM | 0.1666 | 3 | 376.8 | nM | 0.2559 | 4 |
| 8-17 | | | | | | | | | | | | |
| Compound | 17.98 | nM | 0.1072 | 3 | 3533 | nM | 0.1039 | 3 | 41.19 | nM | 0.05 | 3 |
| 8-18 | | | | | | | | | | | | |
| Compound | 100000 | nM | 0 | 2 | 100000 | nM | 0 | 2 | 100000 | nM | 0 | 2 |
| 8-20 | | | | | | | | | | | | |
| Compound | 5.383 | nM | 0.02236 | 4 | 1944 | nM | 0.2964 | 4 | 1302 | nM | 0.2151 | 4 |
| 8-21 | | | | | | | | | | | | |
| Compound | 34.88 | nM | 0.1628 | 2 | 5309 | nM | 0.2028 | 3 | 1650 | nM | 0.2895 | 2 |
| 8-22 | | | | | | | | | | | | |
| Compound | 456.7 | nM | 0.2164 | 3 | 58710 | nM | 0.2022 | 3 | 43260 | nM | 0.2634 | 3 |
| 8-23 | | | | | | | | | | | | |
| Compound | 928.2 | nM | 0.03627 | 3 | 100000 | nM | 0 | 3 | 47470 | nM | 0.1838 | 3 |
| 8-23A | | | | | | | | | | | | |
| Compound | 523.8 | nM | 0.01787 | 3 | 75780 | nM | 0.1704 | 2 | 2101 | nM | 0.1206 | 3 |
| 8-24 | | | | | | | | | | | | |
| Compound | 64.19 | nM | 0.3415 | 4 | 1917 | nM | 0.2013 | 3 | 1627 | nM | 0.1846 | 4 |
| 8-24A | | | | | | | | | | | | |
| Compound | 71.76 | nM | 0.05297 | 3 | 2160 | nM | 0.08828 | 3 | 1588 | nM | 0.0797 | 3 |
| 8-25 | | | | | | | | | | | | |
| Compound | 753.9 | nM | 0.06744 | 3 | 100000 | nM | 0 | 3 | 4457 | nM | 0.1465 | 3 |
| 8-25A | | | | | | | | | | | | |
| Compound | 16.31 | nM | 0.3064 | 7 | 2977 | nM | 0.08509 | 7 | 229.2 | nM | 0.3201 | 8 |
| 8-26 | | | | | | | | | | | | |
| Compound | 10.3 | nM | 0.3134 | 6 | 100000 | nM | 0 | 7 | 191.9 | nM | 0.2332 | 6 |
| 8-26A | | | | | | | | | | | | |
| Compound | 20.28 | nM | 0.1339 | 3 | 3057 | nM | 0.07708 | 3 | 247.9 | nM | 0.0836 | 3 |
| 8-26b | | | | | | | | | | | | |
| Compound | 30.02 | nM | 0.1011 | 3 | 2011 | nM | 0.1821 | 3 | 543.2 | nM | 0.1802 | 3 |
| 8-27 | | | | | | | | | | | | |
| Compound | 2177 | nM | 0.07346 | 4 | 100000 | nM | 0 | 4 | 3406 | nM | 0.06 | 4 |
| 8-28 | | | | | | | | | | | | |
| Compound | 27.33 | nM | 0.08836 | 4 | 6259 | nM | 0.3088 | 4 | 61.4 | nM | 0.1588 | 4 |
| 8-29 | | | | | | | | | | | | |
| Compound | 1723 | nM | 0.1554 | 7 | 6991 | nM | 0.1786 | 4 | 15810 | nM | 0.1975 | 7 |
| 8-30 | | | | | | | | | | | | |
| Compound | 1760 | nM | 0.19 | 3 | 69310 | nM | 0.1731 | 3 | 3360 | nM | 0.095 | 3 |
| 8-31 | | | | | | | | | | | | |
| Compound | 145.2 | nM | 0.1187 | 3 | 72490 | nM | 0.1757 | 2 | 3516 | nM | 0.0821 | 3 |
| 8-31A | | | | | | | | | | | | |
| Compound | .8249 | nM | 0.3871 | 3 | 113.9 | nM | 0.3299 | 2 | 50.55 | nM | 0.2262 | 3 |
| 8-32 | | | | | | | | | | | | |
| Compound | 440.9 | nM | 0.1482 | 3 | 90060 | nM | 0.1104 | 4 | 1574 | nM | 0.1563 | 3 |
| 8-33 | | | | | | | | | | | | |
| Compound | 105.3 | nM | 0.2327 | 3 | 4820 | nM | 0.1503 | 3 | 2388 | nM | 0.2115 | 3 |
| 8-33A | | | | | | | | | | | | |
| Compound | 13.39 | nM | 0.08144 | 3 | 3091 | nM | 0.07578 | 4 | 115.3 | nM | 0.0927 | 3 |
| 8-34 | | | | | | | | | | | | |
| Compound | 3.986 | nM | 0.06378 | 3 | 2665 | nM | 0.08307 | 3 | 169.2 | nM | 0.0638 | 3 |
| 8-35 | | | | | | | | | | | | |
| Compound | 4.167 | nM | 0.1307 | 3 | 2322 | nM | 0.05654 | 3 | 129.6 | nM | 0.2032 | 3 |
| 8-36 | | | | | | | | | | | | |
| Compound | 48.37 | nM | 0.2276 | 3 | 4070 | nM | 0.08935 | 3 | 208.1 | nM | 0.1097 | 3 |
| 8-37 | | | | | | | | | | | | |
| Compound | 9.16 | nM | 0.159 | 3 | 1269 | nM | 0.06511 | 3 | 122.6 | nM | 0.1768 | 3 |
| 8-38 | | | | | | | | | | | | |
| Compound | 15.47 | nM | 0.1099 | 3 | 1784 | nM | 0.1435 | 4 | 128.6 | nM | 0.0497 | 3 |
| 8-39 | | | | | | | | | | | | |
| Compound | 1.796 | nM | 0.3568 | 4 | 294.2 | nM | 0.1463 | 4 | 151.3 | nM | 0.1777 | 4 |
| 8-41 | | | | | | | | | | | | |
| Compound | 2.456 | nM | 0.08592 | 2 | 262.8 | nM | 0.1389 | 2 | 102.5 | nM | 0.15 | 2 |
| 8-41 | | | | | | | | | | | | |
| Compound | 6.756 | nM | 0.08395 | 3 | 776.1 | nM | 0.06092 | 3 | 227.4 | nM | 0.1316 | 3 |
| 8-42 | | | | | | | | | | | | |
| Compound | 2.588 | nM | 0.1439 | 4 | 1631 | nM | 0.05921 | 6 | 23.17 | nM | 0.2312 | 5 |
| 8-43 | | | | | | | | | | | | |
| Compound | 2.035 | nM | 0.1113 | 3 | 820.6 | nM | 0.04809 | 3 | 32.27 | nM | 0.2814 | 3 |
| 8-44 | | | | | | | | | | | | |
| Compound | 17.44 | nM | 0.2173 | 6 | 1316 | nM | 0.1709 | 7 | 339.2 | nM | 0.1394 | 7 |
| 8-45 | | | | | | | | | | | | |
| Compound | 176.5 | nM | 0.1181 | 3 | 4617 | nM | 0.1575 | 3 | 1356 | nM | 0.1887 | 3 |
| 8-46 | | | | | | | | | | | | |
| Compound | 29.27 | nM | 0.3684 | 3 | 3441 | nM | 0.0323 | 3 | 351.4 | nM | 0.1066 | 3 |
| 8-47 | | | | | | | | | | | | |
| Compound | 2.402 | nM | 0.1904 | 3 | 1830 | nM | 0.01001 | 3 | 342.2 | nM | 0.2465 | 3 |
| 8-49 | | | | | | | | | | | | |
| Compound | 3.426 | nM | 0.3324 | 3 | 1140 | nM | 0.1022 | 3 | 388.8 | nM | 0.1852 | 3 |
| 8-50 | | | | | | | | | | | | |
| Compound | 3.058 | nM | 0.2435 | 3 | 457.6 | nM | 0.03327 | 3 | 88.82 | nM | 0.0486 | 3 |
| 8-51 | | | | | | | | | | | | |
| Compound | 39.32 | nM | 0.1091 | 3 | 1554 | nM | 0.1057 | 3 | 457.1 | nM | 0.1934 | 3 |
| 8-52A | | | | | | | | | | | | |
| Compound | 221.8 | nM | 0.1119 | 4 | 57730 | nM | 0.09626 | 4 | 916.7 | nM | 0.077 | 4 |
| 8-54A | | | | | | | | | | | | |
| Compound | 6.562 | nM | 0.1033 | 3 | 161.4 | nM | 0.2081 | 3 | 55.87 | nM | 0.1681 | 3 |
| 8-55 | | | | | | | | | | | | |
| Compound | 61.27 | nM | 0.2923 | 3 | 419.6 | nM | 0.1113 | 3 | 289.7 | nM | 0.0923 | 3 |
| 8-56 | | | | | | | | | | | | |
| Compound | 3.892 | nM | 0.3498 | 4 | 352.9 | nM | 0.1393 | 4 | 45.6 | nM | 0.2334 | 4 |
| 8-57 | | | | | | | | | | | | |
| Compound | 102.2 | nM | 0.426 | 9 | 893.3 | nM | 0.2249 | 9 | 360.4 | nM | 0.2211 | 9 |
| 8-58 | | | | | | | | | | | | |
| Compound | 38.88 | nM | 0.2104 | 3 | 488.6 | nM | 0.09462 | 3 | 584 | nM | 0.1686 | 3 |
| 8-60 | | | | | | | | | | | | |
| Compound | 191.6 | nM | 0.1371 | 3 | 2268 | nM | 0.16 | 3 | 2647 | nM | 0.1823 | 3 |
| 8-61 | | | | | | | | | | | | |
| Compound | 122.3 | nM | 0.1614 | 3 | 805.1 | nM | 0.1068 | 3 | 815.8 | nM | 0.1333 | 3 |
| 8-62 | | | | | | | | | | | | |
| Compound | 120.4 | nM | 0.07218 | 3 | 990.7 | nM | 0.1327 | 3 | 901.3 | nM | 0.0863 | 3 |
| 8-63 | | | | | | | | | | | | |
| Compound | 3405 | nM | 0.2544 | 5 | 100000 | nM | 0 | 5 | 100000 | nM | 0 | 5 |
| 8-64 | | | | | | | | | | | | |
| Compound | 1325 | nM | 0.3768 | 3 | 7866 | nM | 0.2853 | 3 | 3877 | nM | 0.1734 | 3 |
| 8-65 | | | | | | | | | | | | |
| Compound | 2789 | nM | 0.221 | 4 | 20720 | nM | 0.3648 | 4 | 25530 | nM | 0.2753 | 4 |
| 8-66 | | | | | | | | | | | | |
| Compound | 4.239 | nM | 0.3786 | 5 | 663.3 | nM | 0.1295 | 5 | 65.35 | nM | 0.307 | 5 |
| 8-67 | | | | | | | | | | | | |
| Compound | 82.21 | nM | 0.1073 | 3 | 3277 | nM | 0.1155 | 3 | 179 | nM | 0.0848 | 3 |
| 8-69 | | | | | | | | | | | | |
| Compound | 139.9 | nM | 0.3036 | 3 | 3978 | nM | 0.1531 | 3 | 359.5 | nM | 0.1728 | 3 |
| 8-70 | | | | | | | | | | | | |
| Compound | 3729 | nM | 0.01207 | 4 | 100000 | nM | 0 | 4 | 14080 | nM | 0.1721 | 4 |
| 8-71 | | | | | | | | | | | | |
| Compound | 8.987 | nM | 0.3609 | 2 | 506.1 | nM | 0.09761 | 2 | 82.75 | nM | 0.1527 | 2 |
| 8-72 | | | | | | | | | | | | |
| Compound | 452.8 | nM | 0.07481 | 3 | 16280 | nM | 0.2508 | 3 | 623.9 | nM | 0.0632 | 3 |
| 8-74 | | | | | | | | | | | | |
| Compound | 27360 | nM | 0.2151 | 3 | 100000 | nM | 0 | 3 | 39150 | nM | 0.0659 | 3 |
| 8-75 | | | | | | | | | | | | |
| Compound | 100000 | nM | 0 | 3 | 100000 | nM | 0 | 3 | 100000 | nM | 0 | 3 |
| 8-76 | | | | | | | | | | | | |
| Compound | 100000 | nM | 0 | 3 | 100000 | nM | 0 | 3 | 100000 | nM | 0 | 3 |
| 8-77 | | | | | | | | | | | | |
| Compound | 2429 | nM | 0.05044 | 3 | 100000 | nM | 0 | 3 | 100000 | nM | 0 | 3 |
| 8-78 | | | | | | | | | | | | |
| Compound | 6085 | nM | 0.1806 | 3 | 100000 | nM | 0 | 3 | 100000 | nM | 0 | 3 |
| 8-79 | | | | | | | | | | | | |
| Compound | 5377 | nM | 0.06834 | 3 | 100000 | nM | 0 | 3 | 10980 | nM | 0.1283 | 3 |
| 8-81 | | | | | | | | | | | | |
| Compound | 79.76 | nM | 0.3619 | 3 | 5337 | nM | 0.2022 | 3 | 1177 | nM | 0.3586 | 3 |
| 8-82 | | | | | | | | | | | | |
| Compound | 38.86 | nM | 0.02488 | 3 | 2476 | nM | 0.1332 | 3 | 85.77 | nM | 0.1686 | 3 |
| 8-83 | | | | | | | | | | | | |
| Compound | 32.36 | nM | 0.1326 | 3 | 4134 | nM | 0.1905 | 3 | 1827 | nM | 0.0724 | 3 |
| 8-84 | | | | | | | | | | | | |
| Compound | 24.47 | nM | 0.08999 | 3 | 787.6 | nM | 0.1944 | 3 | 485.9 | nM | 0.0397 | 3 |
| 8-86 | | | | | | | | | | | | |
| Compound | 8.139 | nM | 0.1823 | 3 | 1260 | nM | 0.2588 | 3 | 755.8 | nM | 0.2471 | 3 |
| 8-87 | | | | | | | | | | | | |
| Compound | 18.87 | nM | 0.1677 | 3 | 22220 | nM | 0.2113 | 3 | 774.8 | nM | 0.1124 | 3 |
| 8-89 | | | | | | | | | | | | |
| Compound | 6.692 | nM | 0.0044 | 2 | 471.7 | nM | 0.1593 | 2 | 96.85 | nM | 0.2221 | 2 |
| 8-90 | | | | | | | | | | | | |
| Compound | 2.151 | nM | 0.2149 | 2 | 220.5 | nM | 0.18 | 2 | 51.52 | nM | 0.0722 | 2 |
| 8-90A | | | | | | | | | | | | |
| Compound | 1791 | nM | 0.1542 | 4 | 100000 | nM | 0 | 3 | 2482 | nM | 0.2463 | 4 |
| 9-01 | | | | | | | | | | | | |
| Compound | 3.806 | nM | 0.3491 | 3 | 197.1 | nM | 0.1688 | 4 | 52.71 | nM | 0.0512 | 3 |
| 9-02 | | | | | | | | | | | | |
| Compound | 1796 | nM | 0.2453 | 5 | 1907 | nM | 0.1731 | 6 | 1352 | nM | 0.3371 | 4 |
| 9-03 | | | | | | | | | | | | |
| Compound | 6.991 | nM | 0.1568 | 3 | 65.37 | nM | 0.0847 | 3 | 42.9 | nM | 0.0375 | 3 |
| 9-04 | | | | | | | | | | | | |
| Compound | 45.2 | nM | 0.06266 | 3 | 8469 | nM | 0.1832 | 3 | 248.3 | nM | 0.0777 | 3 |
| 9-05 | | | | | | | | | | | | |
| Compound | .9755 | nM | 0.3379 | 4 | 430.7 | nM | 0.2044 | 4 | 24.86 | nM | 0.0773 | 4 |
| 9-06 | | | | | | | | | | | | |
| Compound | 5.982 | nM | 0.3043 | 3 | 665.9 | nM | 0.06478 | 3 | 108.7 | nM | 0.3586 | 3 |
| 9-07 | | | | | | | | | | | | |
| Compound | 16.2 | nM | 0.1202 | 3 | 1032 | nM | 0.1505 | 3 | 139 | nM | 0.102 | 3 |
| 9-08 | | | | | | | | | | | | |
| Compound | 2477 | nM | 0.2113 | 3 | 3590 | nM | 0.1926 | 3 | 7679 | nM | 0.1171 | 3 |
| 9-08A | | | | | | | | | | | | |
| Compound | 175400 | nM | 0.3452 | 2 | 100000 | nM | 0 | 3 | 100000 | nM | 0 | 3 |
| 9-09 | | | | | | | | | | | | |
| Compound | 435.4 | nM | 0.1197 | 3 | 100000 | nM | 0 | 3 | 674.3 | nM | 0.0732 | 3 |
| 9-10 | | | | | | | | | | | | |
| Compound | 830.1 | nM | 0.08905 | 3 | 100000 | nM | 0 | 4 | 22970 | nM | 0.153 | 3 |
| 9-11 | | | | | | | | | | | | |
| Compound | 700.8 | nM | 0.04222 | 3 | 100000 | nM | 0 | 4 | 13260 | nM | 0.1205 | 3 |
| 9-12 | | | | | | | | | | | | |
| Compound | 92.38 | nM | 0.2446 | 3 | 2216 | nM | 0.08069 | 3 | 1043 | nM | 0.1867 | 3 |
| 9-13 | | | | | | | | | | | | |
| Compound | 14.28 | nM | 0.05951 | 3 | 203.5 | nM | 0.1493 | 4 | 387.6 | nM | 0.2241 | 3 |
| 9-14 | | | | | | | | | | | | |
| Compound | 41.97 | nM | 0.3565 | 4 | 278.3 | nM | 0.1693 | 4 | 200.3 | nM | 0.1955 | 4 |
| 9-15 | | | | | | | | | | | | |
| Compound | 94.08 | nM | 0.04751 | 3 | 741.1 | nM | 0.006086 | 3 | 215.4 | nM | 0.0357 | 3 |
| 9-16 | | | | | | | | | | | | |
| Compound | 5.636 | nM | 0.1175 | 3 | 145.6 | nM | 0.1505 | 4 | 66.34 | nM | 0.1224 | 3 |
| 9-17 | | | | | | | | | | | | |
| Compound | .6147 | nM | 0.3686 | 3 | 26.49 | nM | 0.2331 | 3 | 15.25 | nM | 0.0478 | 3 |
| 9-18 | | | | | | | | | | | | |
| Compound | 2.955 | nM | 0.3116 | 3 | 69.92 | nM | 0.06431 | 3 | 27.04 | nM | 0.2106 | 3 |
| 9-19 | | | | | | | | | | | | |
| Compound | 1375 | nM | 0.145 | 3 | 9513 | nM | 0.005455 | 2 | 34190 | nM | 0.1902 | 3 |
| 9-19A | | | | | | | | | | | | |
| Compound | 3.846 | nM | 0.1735 | 3 | 68.47 | nM | 0.1712 | 3 | 35 | nM | 0.1736 | 3 |
| 9-20 | | | | | | | | | | | | |
| Compound | 1.292 | nM | 0.1827 | 11 | 41.33 | nM | 0.1515 | 12 | 50.57 | nM | 0.2174 | 12 |
| 9-21 | | | | | | | | | | | | |
| Compound | 15530 | nM | 0.2525 | 4 | 100000 | nM | 0 | 3 | 71850 | nM | 0.2871 | 4 |
| 9-24 | | | | | | | | | | | | |

Several of the compounds tested showed both strong activity, and significant selectivity for FSHR compared to TSHR and/or LHCGR, as described in Table 2.

### Pharmaceutical Compositions

### Example A-i: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s),optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. A follicle stimulating hormone (FSH) modulator compound of Formula (I): 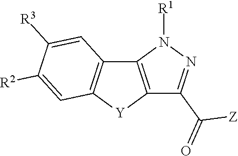
or a pharmaceutically acceptable salt thereof, wherein,
R¹ is C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
Y is —OC(R⁴)₂—;
Z is —OR⁴, —N(R⁴)₂, —SR⁴, —OH, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵;
R² is C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, or —OCH₂CH₃;
R³ is hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁴ is independently hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁵ is independently deuterium, halogen, —OH, —NO₂, —CN, —SR⁶, —S(═O)R⁶, —S(═O)₂R⁶, —N(R⁶)₂, —C(═O)R⁶, —OC(═O)R⁶, —C(═O)OR⁶, —C(═O)N(R⁶)₂, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₃-C₇ cycloalkyl, substituted or unsubstituted C₂-C₇heterocycloalkyl, substituted or unsubstituted aryl, and substituted or unsubstituted heteroaryl; wherein
each R⁶ is independently hydrogen, deuterium, substituted or unsubstituted C₁-C₄ alkyl, -CD₃, substituted or unsubstituted C₁-C₄haloalkyl, substituted or unsubstituted C₁-C₄heteroalkyl, substituted or unsubstituted C₃-C₆ cycloalkyl, substituted or unsubstituted C₂-C₅ heterocycloalkyl, substituted or unsubstituted aryl, or substituted or unsubstituted heteroaryl.

2. An FSH modulator compound of Formula (I): 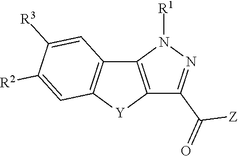
or a pharmaceutically acceptable salt thereof, wherein,
R¹ is C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
Y is —O—, —S—, —S(═O)—, —S(═O)₂—, —NR⁴—, —SC(R⁴)₂—, —C(R⁴)₂O—, —C(R⁴)₂S—, —C(R⁴)₂NR⁴—, —C(R⁴)₂—, —S(═O)C(R⁴)₂—, —C(R⁴)₂S(═O)—, —S(═O)₂C(R⁴)₂—, —C(R⁴)₂S(═O)₂—, or —CR⁴=CR⁴—;
Z is —OR⁴, —N(R⁴)₂, —SR⁴, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₃-C₁₆ alkynyl unsubstituted or substituted with 1, 2, 3, 4, or 5 groups selected from R⁵;
R² is —R, halogen, -haloalkyl, —OR, —SR, —CN, —NO₂, —CF₃, —OCF₃, —SO₂R, —SOR, —C(O)R, —C₂R, —C(O)N(R)₂, —NRC(O)R, —NRC(O)N(R)₂, —NRSO₂R, or —N(R)₂;
R is hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
R³ is hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁴ is independently hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁵ is independently deuterium, halogen, —OH, —NO₂, —CN, —SR⁶, —S(═O)R⁶, —S(═O)₂R⁶, —N(R⁶)₂, —C(═O)R⁶, —OC(═O)R⁶, —C(═O)OR⁶, —C(═O)N(R⁶)₂, unsubstituted C₁-C₆ alkyl, unsubstituted C₂-C₆ alkenyl, unsubstituted C₂-C₆ alkynyl, unsubstituted C₁-C₆ alkoxy, unsubstituted C₃-C₇ cycloalkyl, unsubstituted C₂-C₇heterocycloalkyl, unsubstituted aryl, and unsubstituted heteroaryl; wherein
each R⁶ is independently hydrogen, halogen, deuterium, unsubstituted C₁-C₄ alkyl, -CD₃, unsubstituted C₁-C₄ haloalkyl, unsubstituted C₁-C₄ heteroalkyl, unsubstituted C₃-C₆ cycloalkyl, unsubstituted C₂-C₅ heterocycloalkyl, unsubstituted aryl, or unsubstituted heteroaryl.

3. An FSH modulator compound of Formula (I): 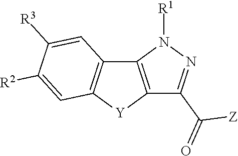
or a pharmaceutically acceptable salt thereof, wherein,
R¹ is C₁-C₁₆ unsubstituted or substituted with 1, 2, 3, 4, or 5 groups selected from R⁵ alkyl, C₁-C₁₆ unsubstituted or substituted with 1, 2, 3, 4, or 5 groups selected from R⁵ alkenyl, C₃-C₁₆ unsubstituted or substituted with 1, 2, 3, 4, or 5 groups selected from R⁵ heteroaryl, unsubstituted or substituted with 1, 2, 3, 4, or 5 groups selected from R⁵ C₆ aryl, unsubstituted or substituted with 1, 2, 3, 4, or 5 groups selected from R⁵ C₃-C₈ cycloalkyl, unsubstituted or substituted with 1, 2, 3, 4, or 5 groups selected from R⁵ C₃-C₈ cycloalkenyl, unsubstituted or substituted with 1, 2, 3, 4, or 5 groups selected from R⁵ C₃-C₈ cycloalkynyl, unsubstituted or substituted with 1, 2, 3, 4, or 5 groups selected from R⁵ heterocycloalkyl, unsubstituted or substituted with 1, 2, 3, 4, or 5 groups selected from R⁵ heterocycloalkenyl, unsubstituted or substituted with 1, 2, 3, 4, or 5 groups selected from R⁵ heterocycloalkynyl, or C₁-C₁₆ unsubstituted or substituted with 1, 2, 3, 4, or 5 groups selected from R⁵ alkynyl;
Y is —O—, —S—, —S(═O)—, —S(═O)₂—, —NR⁴—, —OC(R⁴)₂—, —SC(R⁴)₂—, —C(R⁴)₂O—, —C(R⁴)₂S—, —C(R⁴)₂NR⁴—, —C(R⁴)₂—, —S(═O)C(R⁴)₂—, —C(R⁴)₂S(═O)—, —S(═O)₂C(R⁴)₂—, —C(R⁴)₂S(═O)₂—, —C(R⁴)₂—C(R⁴)₂— or —CR⁴=CR⁴—;
Z is —O-t-butyl;
R² is —R, halogen, -haloalkyl, —OR, —SR, —CN, —NO₂, —CF₃, —OCF₃, —SO₂R, —SOR, —C(O)R, —CO₂R, —C(O)N(R)₂, —NRC(O)R, —NRC(O)N(R)₂, —NRSO₂R, or N(R)₂;
R is hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈cycloalkyl unsubstituted or substituted with 1, 2, 3, 4, or 5 groups selected from R⁵, C₃-C₈cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵:
R³ is hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁴ is independently hydrogen, halogen, C₁-C₆ fluoroalkyl unsubstituted or substituted with 1, 2, 3, 4, or 5 groups selected from R⁵, —O—(C₁-C₆ fluoroalkyl) unsubstituted or substituted with 1, 2, 3, 4, or 5 groups selected from R⁵, —OH, C₁-C₁₆ heteroalkyl unsubstituted or substituted with 1, 2, 3, 4, or 5 groups selected from R⁵, C₁-C₁₆ alkyl unsubstituted or substituted with 1, 2, 3, 4, or 5 groups selected from R⁵, C₁-C₁₆ alkenyl unsubstituted or substituted with 1, 2, 3, 4, or 5 groups selected from R⁵, C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, heterocycloalkynyl unsubstituted or substituted with 1, 2, 3, 4, or 5 groups selected from R⁵, or C₁-C₁₆ alkynyl unsubstituted or substituted with 1, 2, 3, 4, or 5 groups selected from R⁵;
each R⁵ is independently deuterium, halogen, —OH, —NO₂, —CN, —SR⁶, —S(═O)R⁶, —S(═O)₂R⁶, —N(R⁶)₂, —C(═O)R⁶, —OC(═O)R⁶, —C(═O)OR⁶, —C(═O)N(R⁶)₂, unsubstituted C₁-C₆ alkyl, unsubstituted C₂-C₆ alkenyl, unsubstituted C₂-C₆ alkynyl, unsubstituted C₁-C₆ alkoxy, unsubstituted C₃-C₇ cycloalkyl, unsubstituted C₂-C₇heterocycloalkyl, unsubstituted aryl, and unsubstituted heteroaryl; wherein
each R⁶ is independently hydrogen, halogen, deuterium, unsubstituted C₁-C₄ alkyl, -CD₃, unsubstituted C₁-C₄ haloalkyl, unsubstituted C₁-C₄ heteroalkyl, unsubstituted C₃-C₆ cycloalkyl, unsubstituted C₂-C₅ heterocycloalkyl, unsubstituted aryl, or unsubstituted heteroaryl.

4. The compound of claim 1, wherein R³ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or
heterocycloalkynyl unsubstituted or substituted with 1, 2, 3, 4, or 5 groups selected from R⁵.

5. The compound of claim 1, wherein R³ is C₃-C₁₆ heteroaryl unsubstituted or substituted with 1, 2, 3, 4, or 5 groups selected from R⁵.

6. The compound of claim 1, wherein R³ is selected from 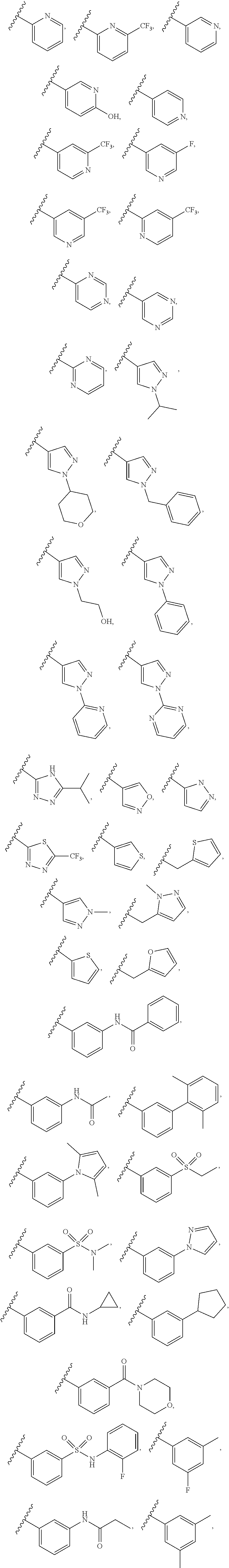 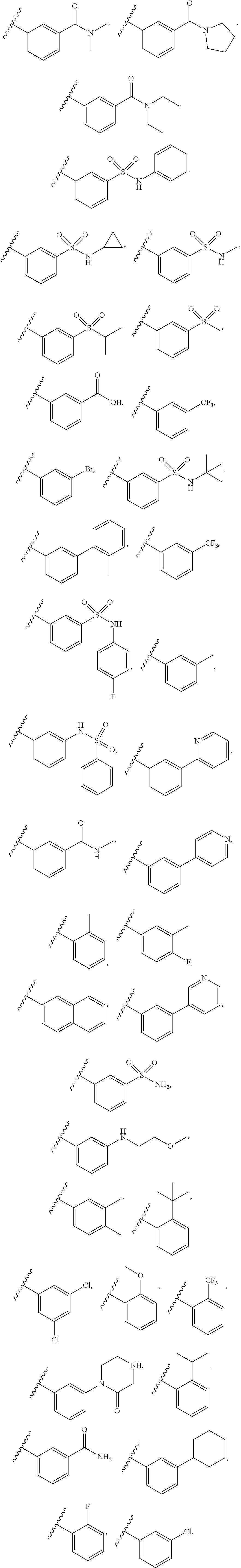 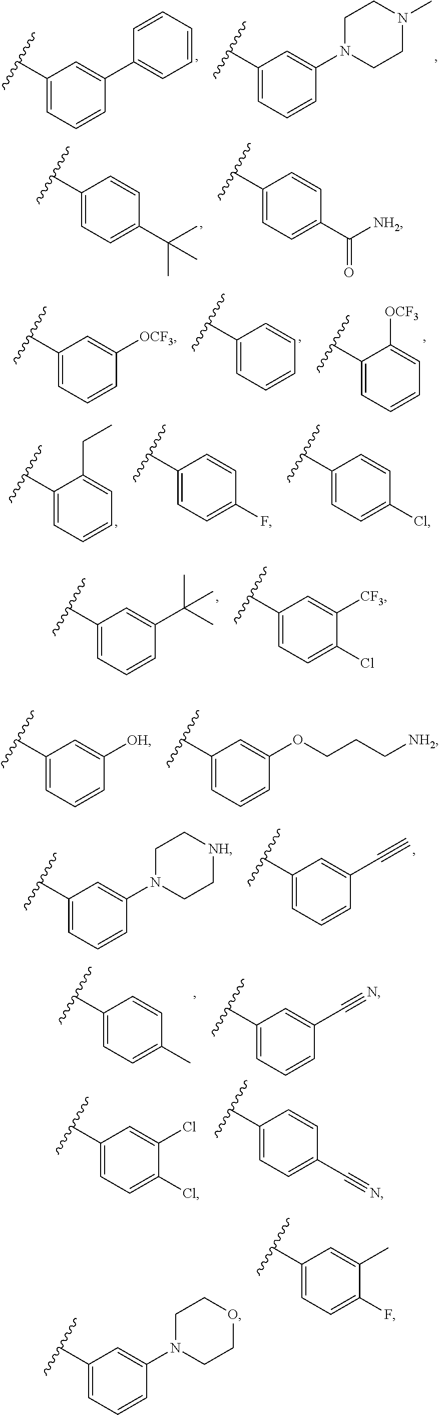 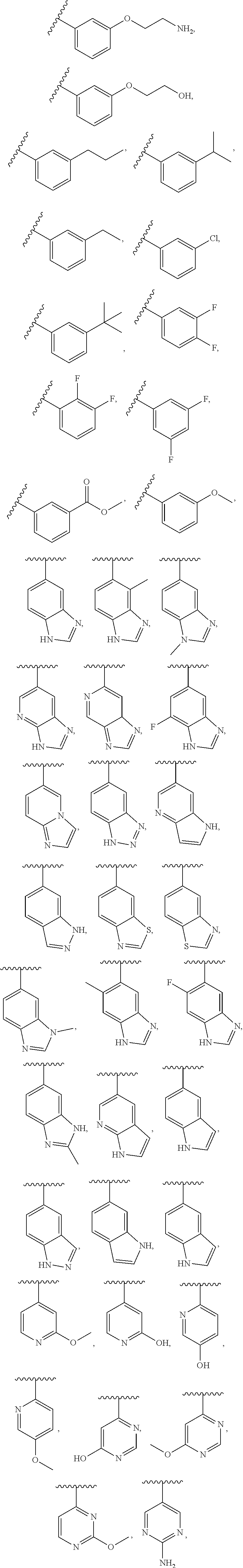 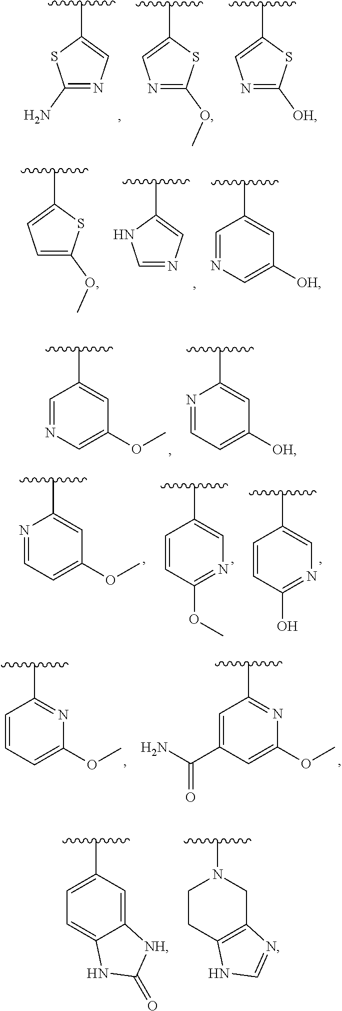
and substitutions thereof.

7. The compound of claim 1, wherein R³ is selected from 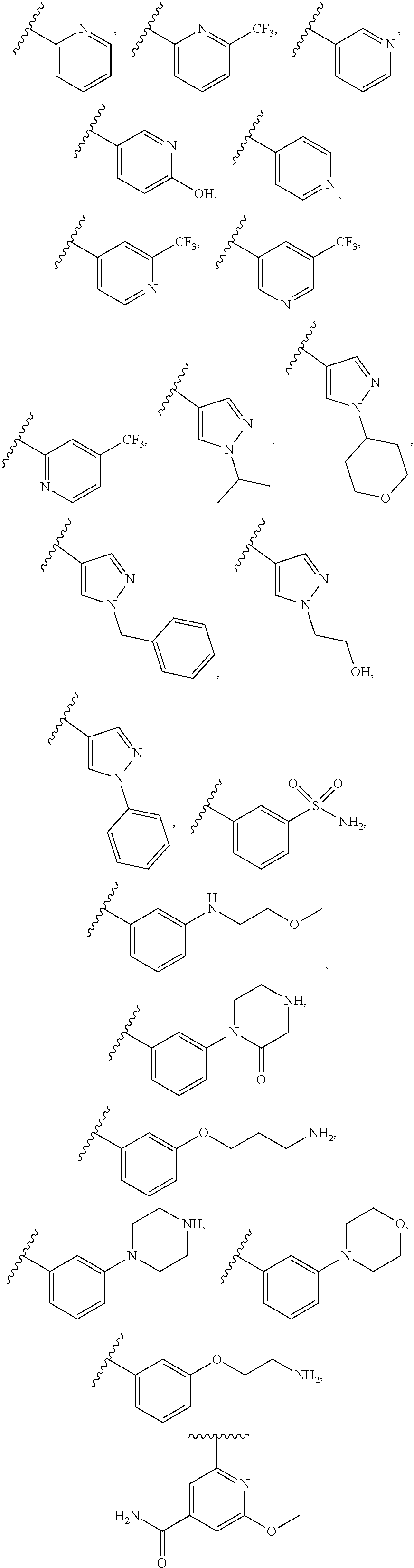
and substitutions thereof.

8. The compound of claim 1, wherein R³ is selected from

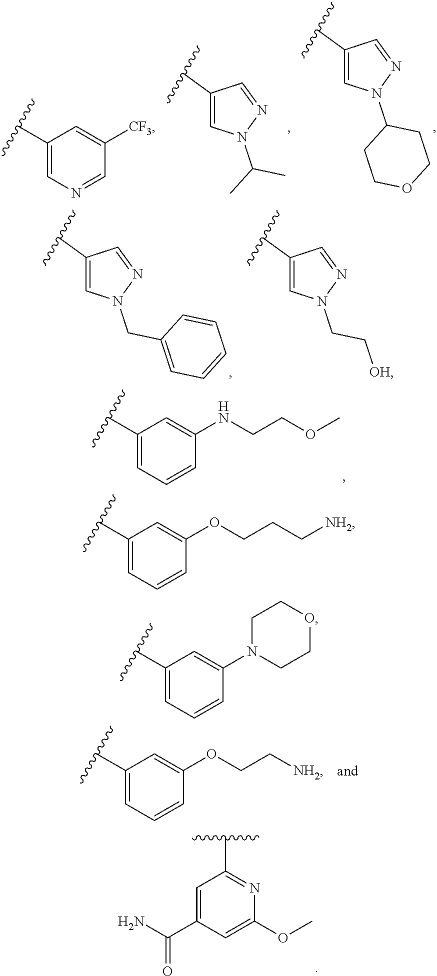

9.-18. (canceled)

19. The compound of claim 2, wherein R² is -halogen, —OR, —SR, —CN, —NO₂, —CF₃, —OCF₃, or —C(═O)CH₃.

20. The compound of claim 2, wherein R² is —OCH₃, —SCH₃, —CN, —NO₂, —CF₃, or —OCF₃.

21.-26. (canceled)

27. The compound of claim 1, wherein R¹ is C₃-C₁₆ heteroaryl unsubstituted or substituted with 1, 2, 3, 4, or 5 groups selected from R⁵, C₆ aryl unsubstituted or substituted with 1, 2, 3, 4, or 5 groups selected from R⁵, C₃-C₈ cycloalkyl unsubstituted or substituted with 1, 2, 3, 4, or 5 groups selected from R⁵, C₃-C₈ cycloalkenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or
heterocycloalkynyl unsubstituted or substituted with 1, 2, 3, 4, or 5 groups selected from R⁵.

28. (canceled)

29. (canceled)

30. The compound of claim 1, wherein R¹ is selected from 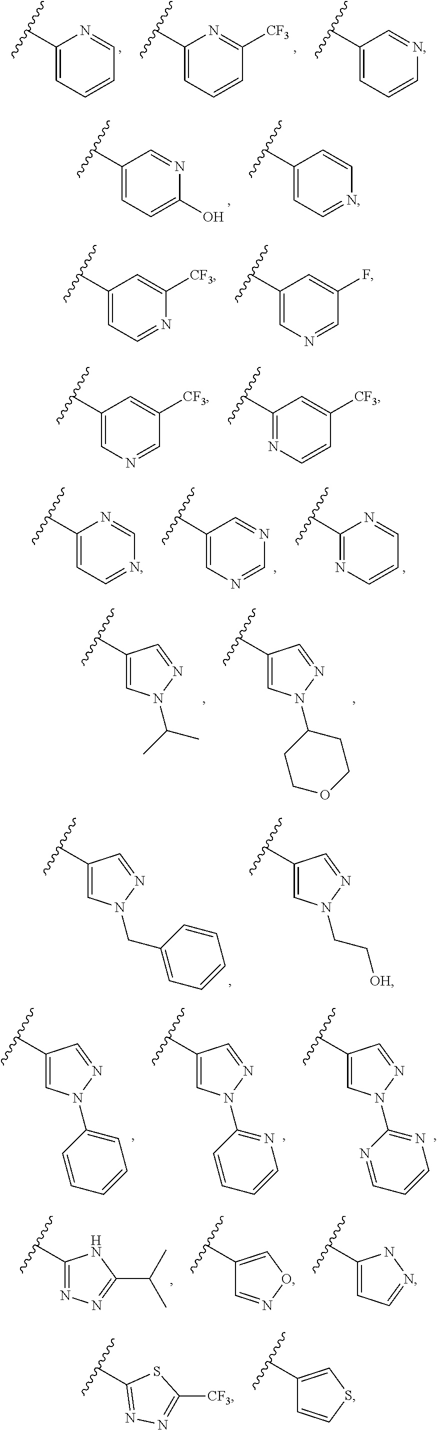 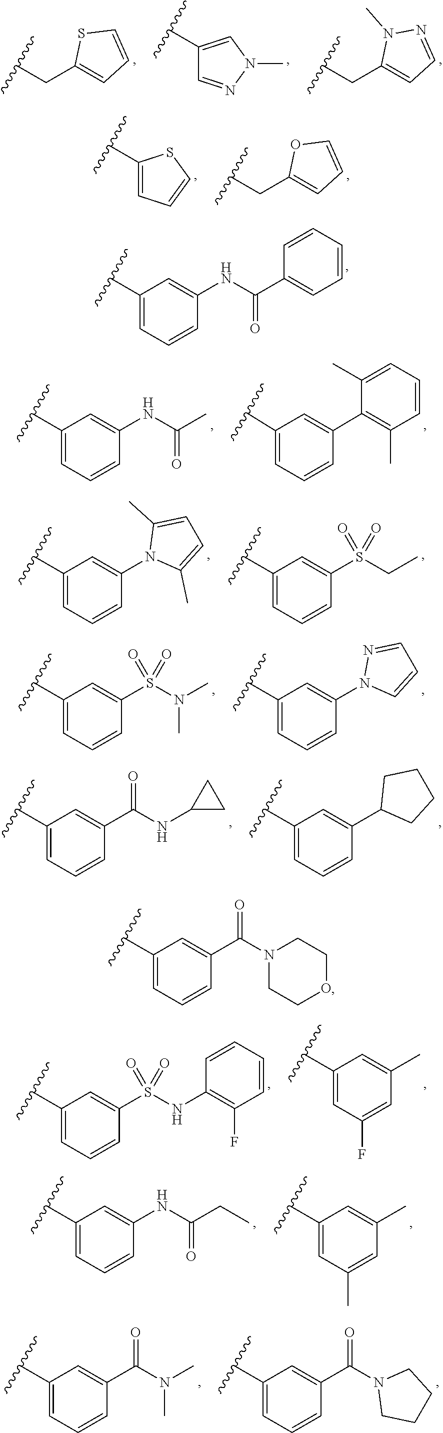 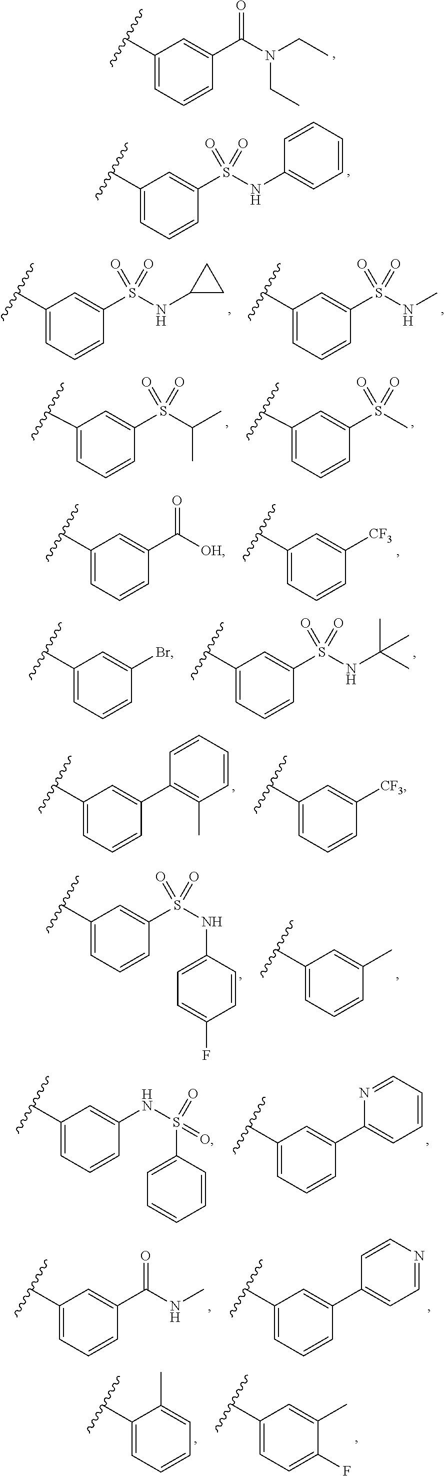 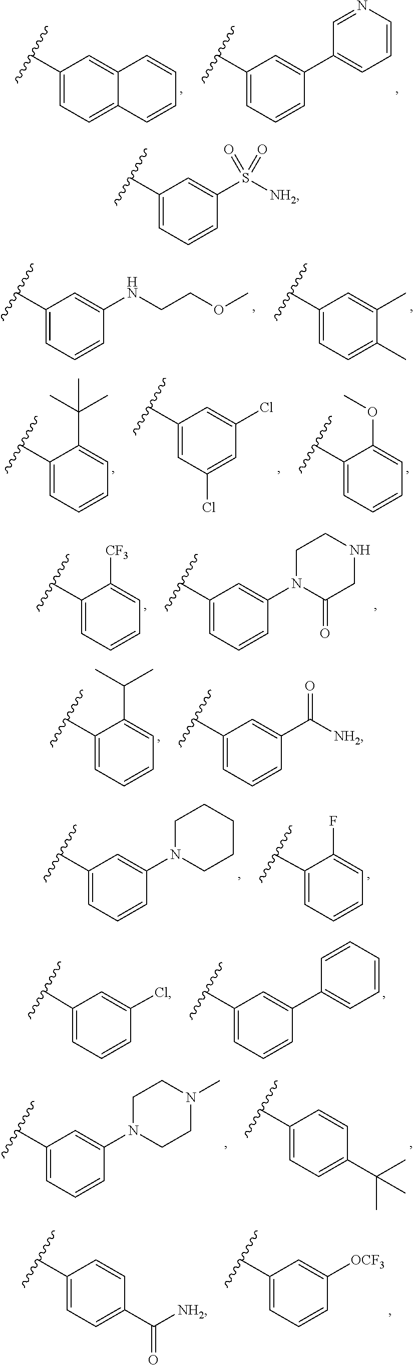 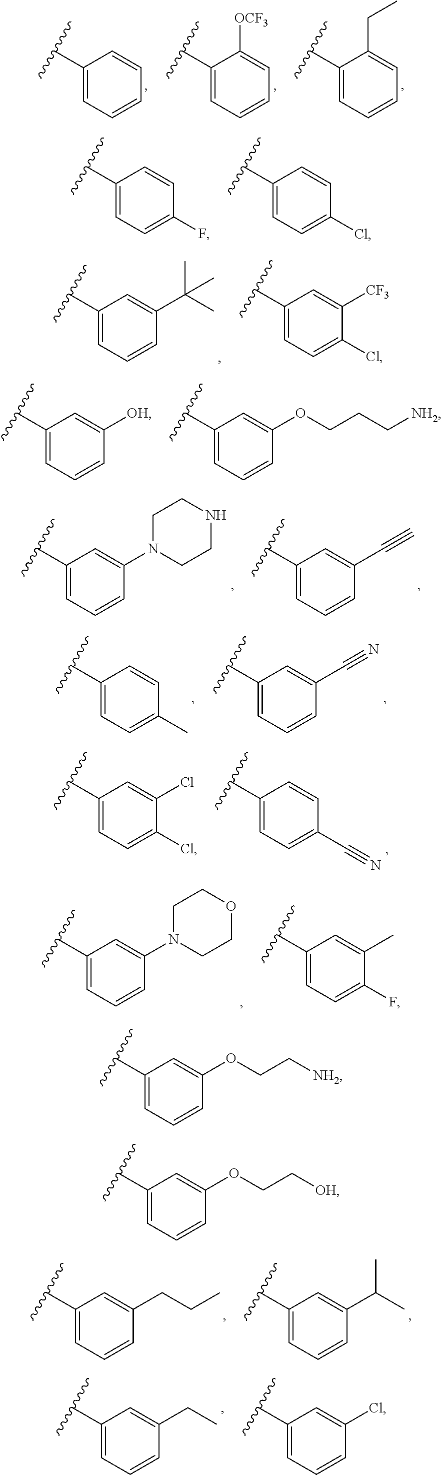 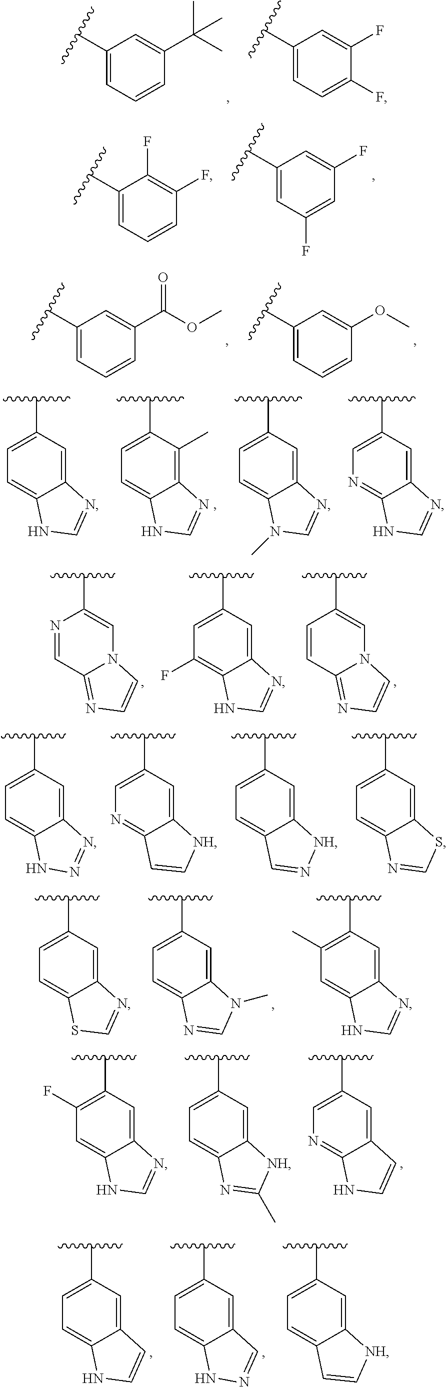 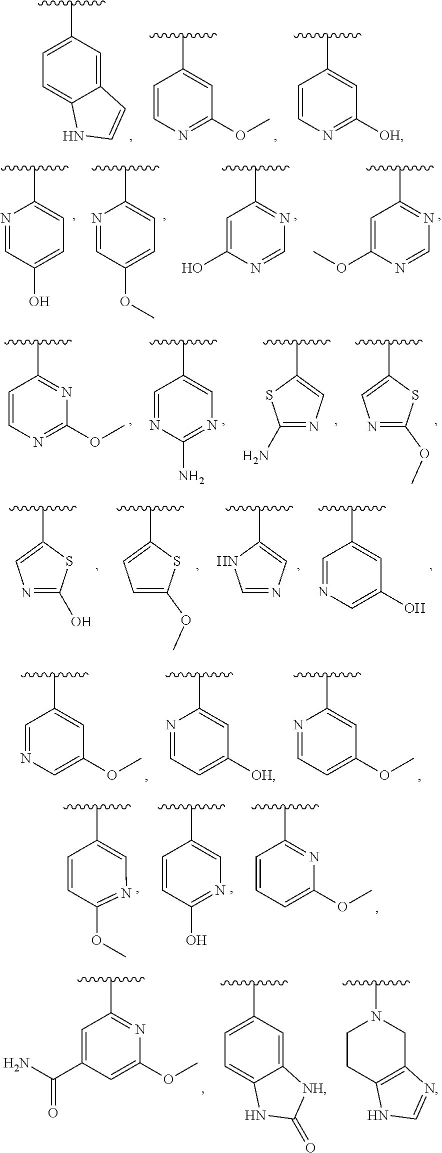
and substitutions thereof.

31. The compound of claim 1, wherein R¹ is selected from 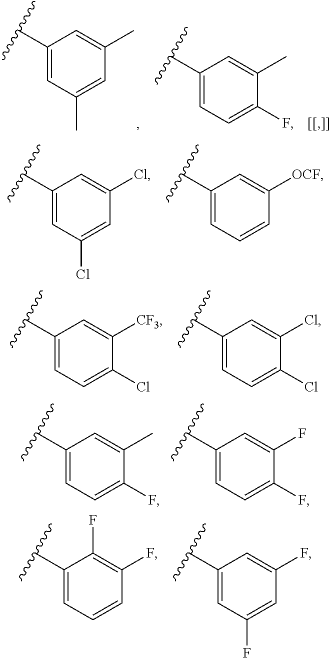
and substitutions thereof.

32.-41. (canceled)

42. The compound of claim 3, wherein Y is —O—, —S—, —NR⁴—, —OC(R⁴)₂—, —SC(R⁴)₂—, —C(R⁴)₂O—, —C(R⁴)₂S—, —C(R⁴)₂NR⁴—, —C(R⁴)₂—, —S(═O)C(R⁴)₂—, —C(R⁴)₂S(═O)—, —S(═O)₂C(R⁴)₂—, —C(R⁴)₂S(═O)₂—, or —CR⁴=CR⁴—.

43. (canceled)

44. The compound of claim 3, wherein Y is —O—, —S—, —NH—, —OCH₂—, —SCH₂—, —CH₂O—, —CH₂S—, —CH₂—, —S(═O)CH₂—, —CH₂S(═O)—, —S(═O)₂CH₂—, or —CH₂S(═O)₂—.

45.-74. (canceled)

75. The compound of claim 1, wherein Z is —OR⁴, —N(R⁴)₂, —SR⁴, —CF₃, —OCF₃, —OH, C₃-C₈ cycloalkyl unsubstituted or substituted with 1, 2, 3, 4, or 5 groups selected from R⁵, C₃-C₈ cycloalekenyl unsubstituted or substituted with 1, 2, 3, 4, or 5 groups selected from R⁵, C₃-C₈ cycloalkynyl unsubstituted or substituted with 1, 2, 3, 4, or 5 groups selected from R⁵, heterocycloalkyl unsubstituted or substituted with 1, 2, 3, 4, or 5 groups selected from R⁵, heterocycloalkenyl unsubstituted or substituted with 1, 2, 3, 4, or 5 groups selected from R⁵, or heterocycloalkynyl unsubstituted or substituted with 1, 2, 3, 4, or 5 groups selected from R⁵.

76. The compound of claim 1, wherein Z is —OR⁴, —N(R⁴)₂, —SR⁴, —CF₃, —OCF₃, or is selected from

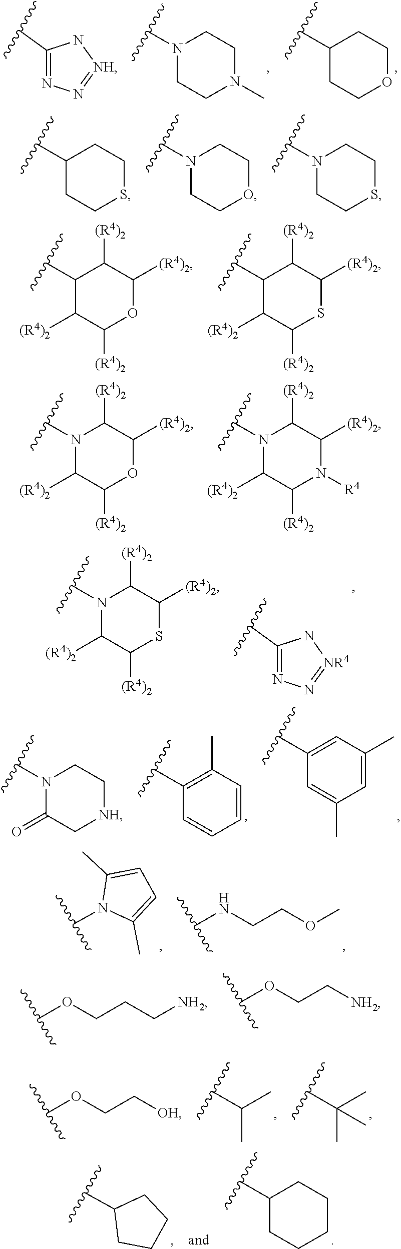

77. (canceled)

78. The compound of claim 1, wherein Z is —OR⁴, —N(R⁴)₂, —SR⁴, and at least one R⁴ of Z is selected from

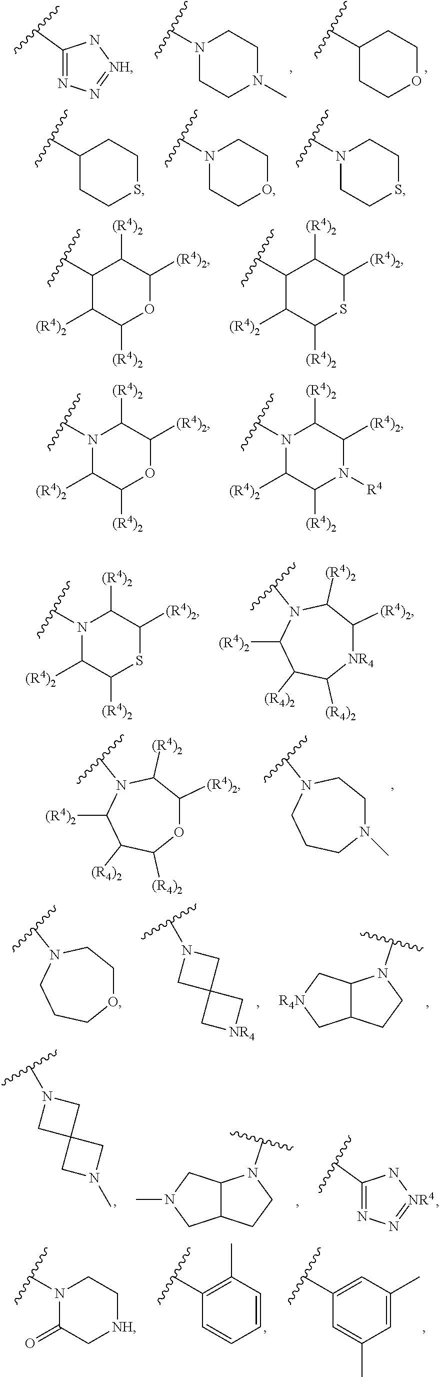

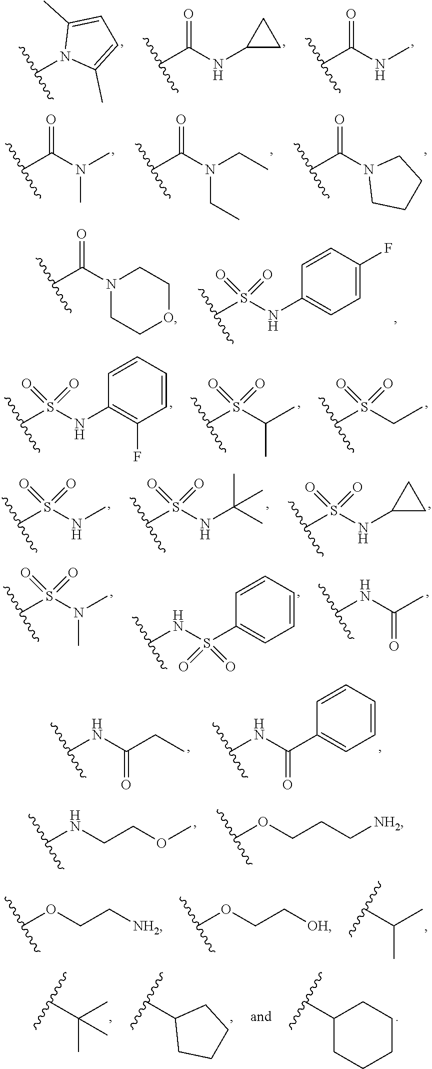

79. The compound of claim 1, wherein Z is —OR⁴ or —SR⁴, and the R⁴ of Z is

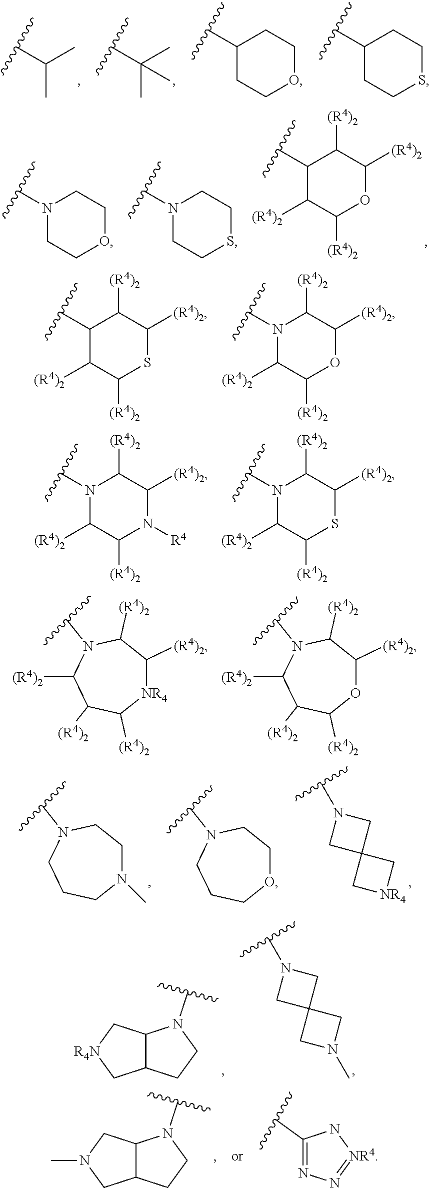

80. The compound of claim 1, wherein Z is selected from

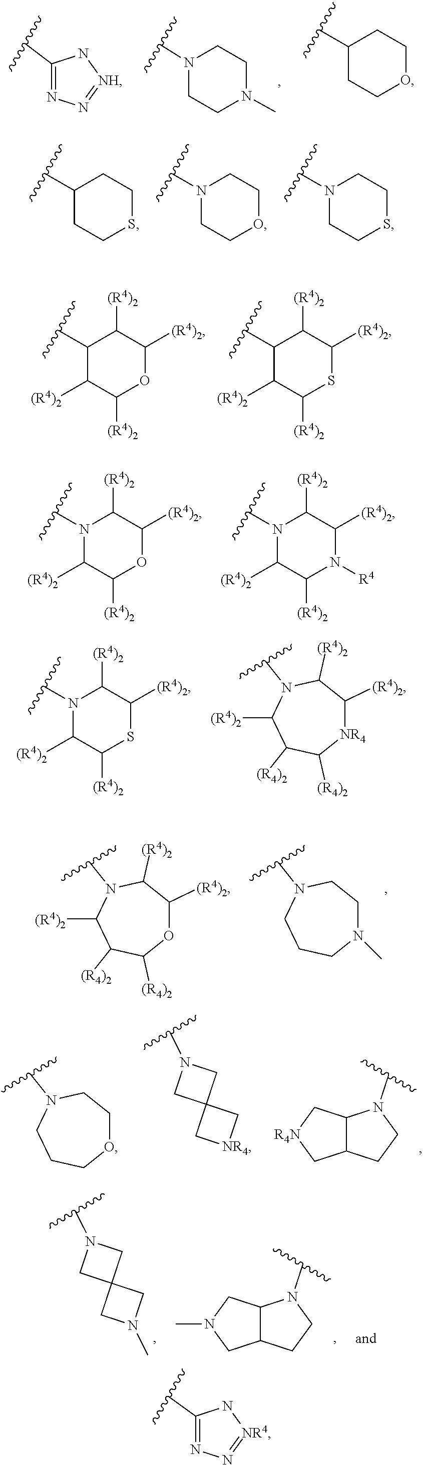

81.-127. (canceled)
